(19) United States
(12) Patent Application Publication
(10) Pub. No.: US 2023/0309380 A1
(43) Pub. Date: Sep. 28, 2023

(54) COMPOUND, MATERIAL FOR ORGANIC ELECTROLUMINESCENCE DEVICE, ORGANIC ELECTROLUMINESCENCE DEVICE, AND ELECTRONIC APPARATUS

(71) Applicant: IDEMITSU KOSAN CO.,LTD., Tokyo (JP)

(72) Inventors: Sayaka MIZUTANI, Tokyo (JP); Masato MITANI, Tokyo (JP); Ryota TAKAHASHI, Tokyo (JP); Masatoshi SAITO, Tokyo (JP)

(73) Assignee: IDEMITSU KOSAN CO.,LTD., Tokyo (JP)

(21) Appl. No.: 17/957,084

(22) Filed: Sep. 30, 2022

(30) Foreign Application Priority Data
Sep. 30, 2021 (JP) 2021-160986

(51) Int. Cl. H01L 51/00; C07D 235/08

(52) U.S. Cl. CPC H01L 51/0052; C07D 235/08; H01L 51/0072; H01L 51/508

(57) ABSTRACT

A compound represented by the following formula (1).

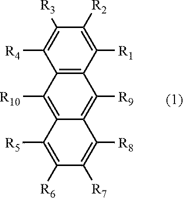

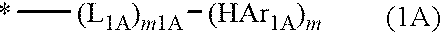

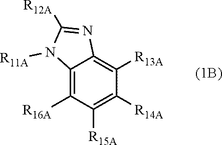

### TECHNICAL FIELD

Embodiments described in the present specification generally relate to a novel compound, a material for an organic electroluminescence device, an organic electroluminescence device, and an electronic apparatus.

### BACKGROUND ART

When voltage is applied to an organic electroluminescence device (hereinafter, also referred to as an organic EL device), holes and electrons are injected into an emitting layer from an anode and a cathode, respectively. Then, thus injected holes and electrons are recombined in the emitting layer, and excitons are formed therein.

Conventional organic EL devices have not yet had sufficient device performance. Although materials used for the organic EL device are gradually improved to enhance the device performance, further performance enhancement is required.

Patent Documents 1 to 3 disclose a compound having the specific structure capable of being used for an electron-transporting region provided between an emitting layer and a cathode of an organic EL device.

### Related Art Documents

### Patent Documents

[Patent Document 1] WO 2020/000921 A1
[Patent Document 2] CN 109879812 A
[Patent Document 3] KR 10-2017-107140

### SUMMARY OF THE INVENTION

It is an object of the present invention to provide a compound capable of achieving an organic EL device having higher performance.

As a result of extensive studies, the present inventors have found that an organic EL device having higher performance is capable of being achieved, when a compound having the specific structure is used, and has completed the present invention.

According to the present invention, the following compound and the like are provided.

1. A compound represented by the following formula (1):

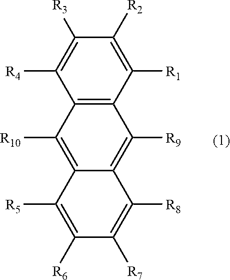

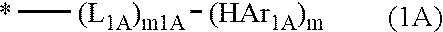

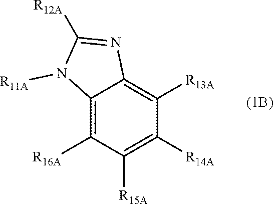

wherein in the formula (1),
at least one of R₁ to R₁₀ is a group represented by the formula (1A);
one or more sets of the adjacent two or more of R₁ to R₁₀ which are not the group represented by the formula (1A) form a substituted or unsubstituted, saturated or unsaturated ring by bonding with each other, or do not bond with each other;
R₁ to R₁₀ which are not the group represented by the formula (1A) and which do not bond with each other are independently a hydrogen atom or a substituent X;
the substituent X is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₅ are present, the two or more of each of R₉₀₁ to R₉₀₅ may be the same as or different from each other;
   when two or more substituents X are present, the two or more substituents X may be the same as or different from each other;
in the formula (1A),
HAr_{1A} is a group represented by the formula (1B); m is an integer of 1 to 5; when two or more HAr_{1A}’s are present, the two or more HAr_{1A}’s may be the same as or different from each other;
L_{1A} is
a substituted or unsubstituted aromatic hydrocarbon group having 6 to 30 ring carbon atoms, or
a substituted or unsubstituted heterocyclic group having 5 to 30 ring atoms;
m1A is an integer of 0 to 3; when m1A is 0, m is 1 and HAr_{1A} is directly bonded with the anthracene skeleton in the formula (1) via a single bond; when m1A is 2 or 3, a plurality of L_{1A}’_{S} is linked in series with each other and HAr_{1A} is bonded with L_{1A} which is farthest from the anthracene skeleton; when two or more L_{1A}’s are present, the two or more L_{1A}’s may be the same as or different from each other;
one or more sets of the adjacent two or more of m1A L_{1A}’s and m HAr_{1A}’s do not form a substituted or unsubstituted, saturated or unsaturated ring;
in the formula (1B),
any one of R_{11A} to R_{16A} represents a bond with L_{1A} in the formula (1A); when m1A is 0, any of carbon atoms and nitrogen atoms in the benzimidazole and any of carbon atoms in the anthracene of the formula (1) are directly bonded via a single bond;
R_{11A} to R_{16A} which do not represent a bond with L_{1A} are independently a hydrogen atom or a substituent Y; one or more sets of the adjacent two or more of R_{11A} to R_{16A} which do not represent a bond with L_{1A} do not bond with each other;
the substituent Y is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are the same as defined in the formula (1);
R₉₀₆ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₆ to R₉₀₇ are present, the two or more of each of R₉₀₆ to R₉₀₇ may be the same as or different from each other;
when two or more substituents Y are present, the two or more substituents Y may be the same as or different from each other;
provided that the compound represented by the formula (1) satisfies all of the following conditions 1 to 6;
condition 1: the compound represented by the formula (1) comprises at least one deuterium atom;
condition 2: at least one of R₉ and R₁₀ is the group represented by the formula (1A) or the substituent X;
condition 3: when at least one of R₁ to R₈ in the formula (1) is the substituent X, m is 1 in the formula (1A) and the substituent in the case of “substituted or unsubstituted” wherein L_{1A} is a substituted or unsubstituted phenyl group is not a substituted or unsubstituted benzimidazolyl group;
condition 4: when at least one of R₁ to R₈ in the formula (1) is a substituted or unsubstituted naphthyl group, R_{12A} in the formula (1B) do not represent a bond with L_{1A} in the formula (1A);
condition 5: when at least one of R₂, R₃, R₆, and R₇ in the formula (1) is the group represented by the formula (1A), at least one hydrogen atom selected from the group consisting of, R₁ to R₁₀ which are hydrogen atoms, hydrogen atoms possessed by the substituted or unsubstituted, saturated or unsaturated ring formed in the case where one or more sets of the adjacent two or more of R₁ to R₁₀ bond with each other, hydrogen atoms possessed by R₁ to R₁₀ which are the substituents X, and hydrogen atoms possessed by L_{1A} is a deuterium atom; and
condition 6: when any one of R_{14A} and R_{15A} in the formula (1B) represents a bond with L_{1A} in the formula (1A), m1A is not 0.

2. An organic electroluminescence device comprising a cathode,
an emitting layer,
an electron-transporting region, and
an anode in this order,
wherein the electron-transporting region comprises a compound represented by the following formula (101):

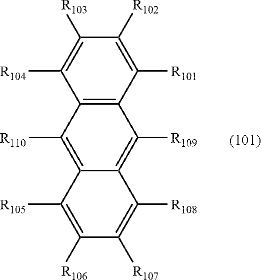

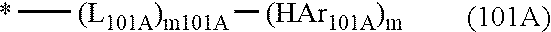

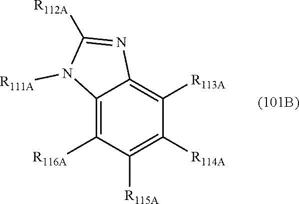

wherein in the formula (101),
at least one of R₁₀₁ to R₁₁₀ is a group represented by the formula (101A);
one or more sets of the adjacent two or more of R₁₀₁ to R₁₁₀ which are not the group represented by the formula (101A) form a substituted or unsubstituted, saturated or unsaturated ring by bonding with each other, or do not bond with each other;
R₁₀₁ to R₁₁₀ which are not the group represented by the formula (101A) and which do not bond with each other are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
in the formula (101A),
HAr_{101A} is a group represented by the formula (101B); m is an integer of 1 to 5; when two or more HAr_{101A}’s are present, the two or more HAr_{101A}’s may be the same as or different from each other;
L_{101A} is
a substituted or unsubstituted aromatic hydrocarbon group having 6 to 30 ring carbon atoms, or
a substituted or unsubstituted heterocyclic group having 5 to 30 ring atoms;
m101A is an integer of 0 to 3; when m101A is 0, m is 1 and HAr_{101A} is directly bonded with the anthracene skeleton in the formula (101) via a single bond; when m101A is 2 or 3, a plurality of L_{101A}’s is linked in series with each other and HAr_{101A} is bonded with L_{101A} which is farthest from the anthracene skeleton; when two or more L_{101A}’s are present, the two or more L_{101A}’s may be the same as or different from each other;
one or more sets of the adjacent two or more of m101AL_{101A}’s and m HAr_{101A}’s do not form a substituted or unsubstituted, saturated or unsaturated ring;
in the formula (101B),
any one of R_{111A} to R_{116A} represents a bond with L_{101A} in the formula (101A); whenm101A is 0, any of carbon atoms and nitrogen atoms in the benzimidazole and any of carbon atoms in the anthracene of the formula (101) are directly bonded via a single bond;
R_{111A} to R_{116A} which do not represent a bond with L_{101A} are independently a hydrogen atom, or a substituent R; one or more sets of the adjacent two or more of R_{111A} to R_{116A} which do not represent a bond with L_{101A} do not bond with each other;
the substituent R is the same as defined in R₁₀₁ to R₁₁₀;
provided that the compound represented by the formula (101) comprises at least one deuterium atom.

3. An electronic apparatus, comprising the organic electroluminescence device according to 2.

According to the present invention, there can be provided a compound capable of achieving an organic EL device having higher performance.

### BRIEF DESCRIPTION OF THE DRAWINGS

The FIGURE is a diagram showing a schematic configuration of an organic EL device according to an aspect of the present invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

### Substituent as Described in This Specification

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### Substituted or Unsubstituted Aryl Group

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “ryl group unsubstituted by a substituent”, and the substituted aryl group refers to the case where the “substituted or unsubstituted aryl group” is an” aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

Unsubstituted aryl group (specific example group G1A):
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

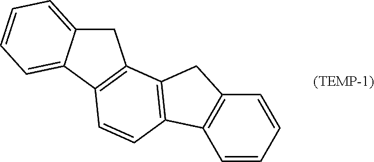

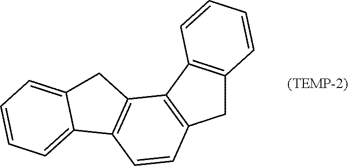

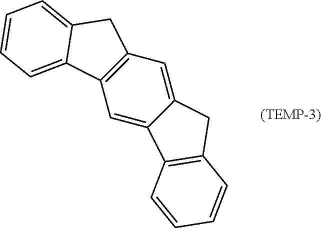

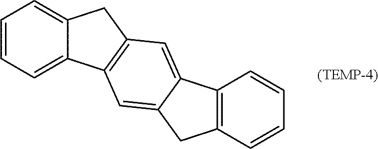

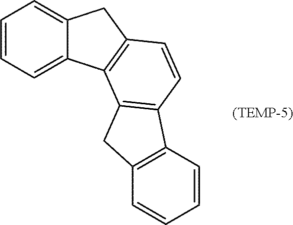

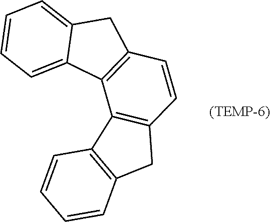

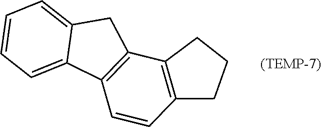

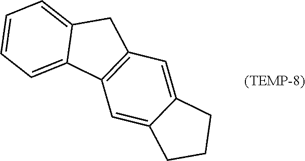

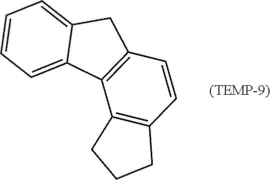

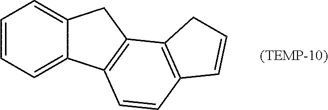

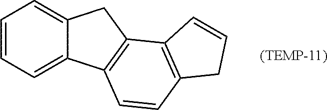

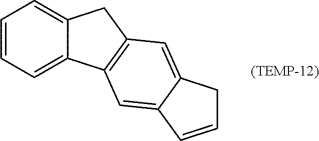

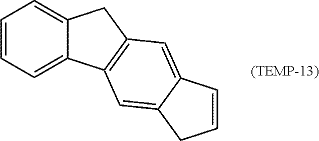

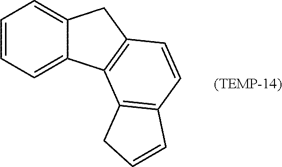

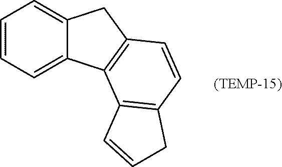

Substituted aryl group (specific example group G1B):
an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring
structures represented by any of the general formulas (TEMP-1) to (TEMP-15) are substituted by a substituent.

### Substituted or Unsubstituted Heterocyclic Group

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent” and the substituted heterocyclic group refers to the case where the “substituted or unsubstituted heterocyclic group”is a “heterocyclic group substituted by a substituent”). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B 1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

Unsubstituted heterocyclic group containing a nitrogen atom (specific example group G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

Unsubstituted heterocyclic group containing an oxygen atom (specific example group G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

Unsubstituted heterocyclic group containing a sulfur atom (specific example group G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent heterocyclic group derived by removing one hydrogen atom from the ring structures represented by any of the following general formulas (TEMP-16) to (TEMP-33) (specific example group G2A4):

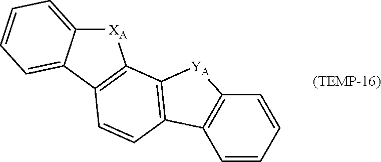

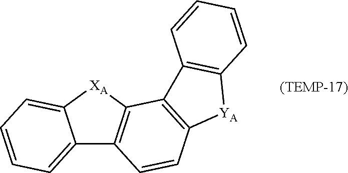

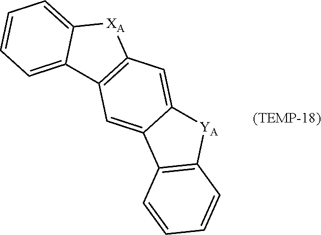

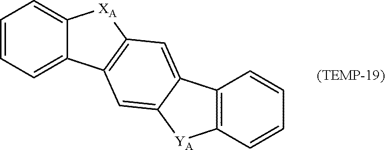

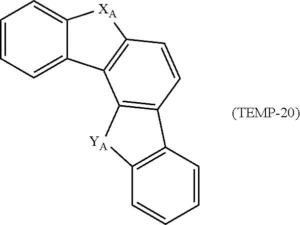

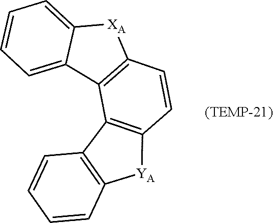

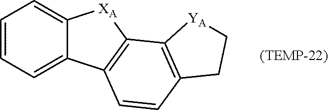

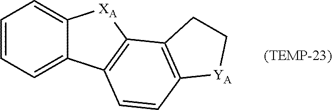

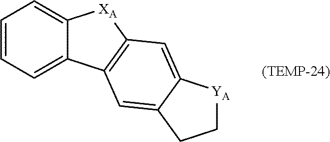

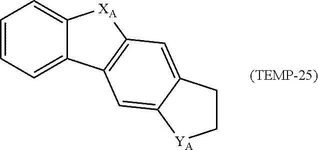

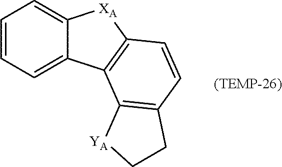

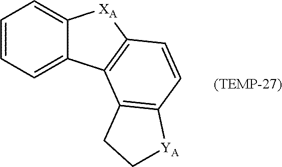

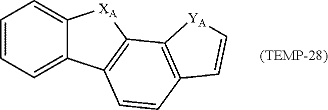

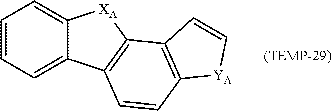

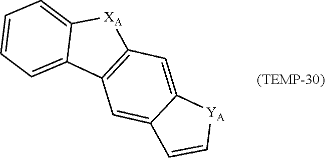

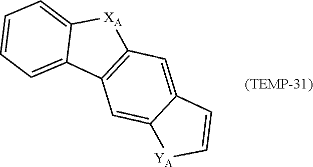

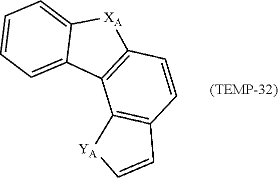

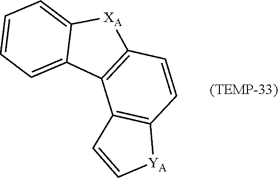

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.

Substituted heterocyclic group containing a nitrogen atom (specific example group G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

Substituted heterocyclic group containing an oxygen atom (specific example group G2B2):
a phenyldibenzofuranyl group,
a methyldibenzofuranyl group,
a t-butyldibenzofuranyl group, and
a monovalent residue of spiro[9H-xanthene-9,9′-[9H]fluorene].

Substituted heterocyclic group containing a sulfur atom (specific example group G2B3):
a phenyldibenzothiophenyl group,
a methyldibenzothiophenyl group,
a t-butyldibenzothiophenyl group, and
a monovalent residue of spiro[9H-thioxanthene-9,9′-[9H]fluorene].

Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

### Substituted or Unsubstituted Alkyl Group

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent” and the substituted alkyl group refers to the case where the “substituted or unsubstituted alkyl group” is an “alkyl group substituted by a substituent”). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

Unsubstituted alkyl group (specific example group G3A):
a methyl group,
an ethyl group,
a n-propyl group,
an isopropyl group,
a n-butyl group,
an isobutyl group,
a s-butyl group, and
a t-butyl group.

Substituted alkyl group (specific example group G3B):
a heptafluoropropyl group (including isomers),
a pentafluoroethyl group,
a 2,2,2-trifluoroethyl group, and
a trifluoromethyl group.

### Substituted or Unsubstituted Alkenyl Group

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent” and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

Unsubstituted alkenyl group (specific example group G4A):
a vinyl group,
an allyl group,
a 1-butenyl group,
a 2-butenyl group, and
a 3-butenyl group.

Substituted alkenyl group (specific example group G4B):
a 1,3-butanedienyl group,
a 1-methylvinyl group,
a 1-methylallyl group,
a 1,1-dimethylallyl group,
a 2-methylally group, and
a 1,2-dimethylallyl group.

### Substituted or Unsubstituted Alkynyl Group

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

Unsubstituted alkynyl group (specific example group G5A):

an ethynyl group.

### Substituted or Unsubstituted Cycloalkyl Group

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent” and the substituted cycloalkyl group refers to the case where the “substituted or unsubstituted cycloalkyl group” is a “cycloalkyl group substituted by a substituent”). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

Unsubstituted cycloalkyl group (specific example group G6A):
a cyclopropyl group,
a cyclobutyl group,
a cyclopentyl group,
a cyclohexyl group,
a 1-adamantyl group,
a 2-adamantyl group,
a 1-norbomyl group, and
a 2-norbomyl group.

Substituted cycloalkyl group (specific example group G6B):

a 4-methylcyclohexyl group.

### Group Represented by -Si (R₉₀₁)(R₉₀₂)(R₉₀₃)

Specific examples of the group represented by -Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1’s in —Si(G1)(G1)(G1) are the same or different.
Plural G2’s in —Si(G1)(G2)(G2) are the same or different.
Plural G1’s in —Si(G1)(G1)(G2) are the same or different.
Plural G2’s in —Si(G2)(G2)(G2) are be the same or different.
Plural G3’s in —Si(G3)(G3)(G3) are the same or different.
Plural G6’s in —Si(G6)(G6)(G6) are be the same or different.

### Group Represented by —O—(R₉₀₄)

Specific examples of the group represented by —O—(R₉₀₄) in this specification (specific example group G8) include:
—O(G1),
—O(G2),
—O(G3), and
—O(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.

### Group Represented by —S—(R₉₀₅)

Specific examples of the group represented by —S—(R₉₀₅) in this specification (specific example group G9) include:
—S(G1),
—S(G2),
—S(G3), and
—S(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.

### Group Represented by -N(R₉₀₆)(R₉₀₇)

Specific examples of the group represented by -N(R₉₀₆)(R₉₀₇) in this specification (specific example group G10) include:
—N(G1)(G1),
—N(G2)(G2),
—N(G1)(G2),
—N(G3)(G3), and
—N(G6)(G6).
G1 is the “substituted or unsubstituted aryl group” described in the specific example group G1.
G2 is the “substituted or unsubstituted heterocyclic group” described in the specific example group G2.
G3 is the “substituted or unsubstituted alkyl group” described in the specific example group G3.
G6 is the “substituted or unsubstituted cycloalkyl group” described in the specific example group G6.
Plural G1’s in —N(G1)(G1) are the same or different.
Plural G2’s in —N(G2)(G2) are the same or different.
Plural G3’s in —N(G3)(G3) are the same or different.
Plural G6’s in —N(G6)(G6) are the same or different.

### Halogen Atom

Specific examples of the “halogen atom” described in this specification (specific example group G11) include a fluorine atom, a chlorine atom, a bromine atom, an iodine atom, and the like.

### Substituted or Unsubstituted Fluoroalkyl Group

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

### Substituted or Unsubstituted Haloalkyl Group

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

### Substituted or Unsubstituted Alkoxy Group

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

### Substituted or Unsubstituted Alkylthio Group

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

### Substituted or Unsubstituted Aryloxy Group

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

### Substituted or Unsubstituted Arylthio Group

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

### Substituted or Unsubstituted Trialkylsilyl Group

Specific examples of the “trialkylsilyl group” described in this specification include a group represented by —Si(G3)(G3)(G3), where G3 is the “substituted or unsubstituted alkyl group” described in the specific example group G3. Plural G3’s in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

### Substituted or Unsubstituted Aralkyl Group

Specific examples of the “substituted or unsubstituted aralkyl group” described in this specification is a group represented by —(G3)—(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

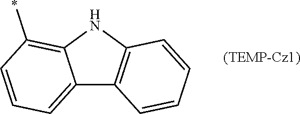 [SMILES: *c1cccc2c1[nH]c1ccccc12]

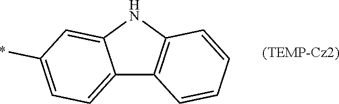 [SMILES: *c1ccc2c(c1)[nH]c1ccccc12]

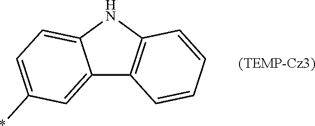 [SMILES: *c1ccc2[nH]c3ccccc3c2c1]

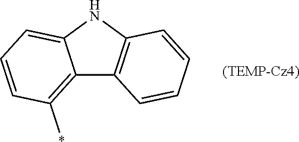 [SMILES: *c1cccc2[nH]c3ccccc3c12]

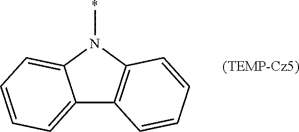 [SMILES: *n1c2ccccc2c2ccccc21]

In this specification, the (9-phenyl)carbazolyl group is specifically any of the following groups, unless otherwise specified in this specification.

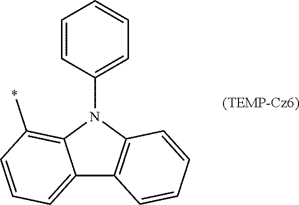 [SMILES: *c1cccc2c3ccccc3n(-c3ccccc3)c12]

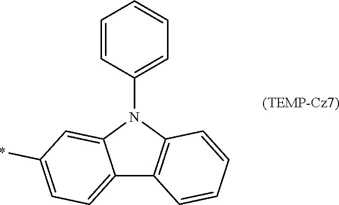 [SMILES: *c1ccc2c3ccccc3n(-c3ccccc3)c2c1]

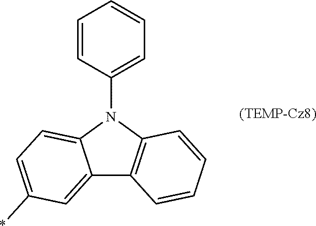 [SMILES: *c1ccc2c(c1)c1ccccc1n2-c1ccccc1]

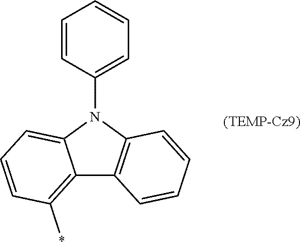 [SMILES: *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

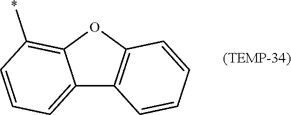 [SMILES: *c1cccc2c1oc1ccccc12]

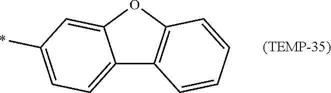 [SMILES: *c1ccc2c(c1)oc1ccccc12]

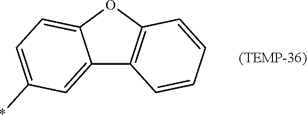 [SMILES: *c1ccc2oc3ccccc3c2c1]

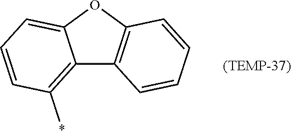 [SMILES: *c1cccc2oc3ccccc3c12]

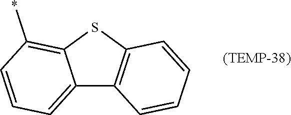 [SMILES: *c1cccc2c1sc1ccccc12]

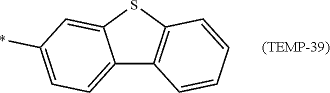 [SMILES: *c1ccc2c(c1)sc1ccccc12]

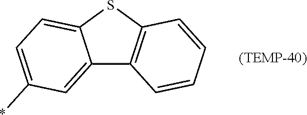 [SMILES: *c1ccc2sc3ccccc3c2c1]

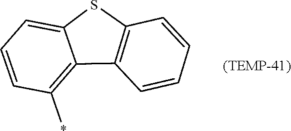 [SMILES: *c1cccc2sc3ccccc3c12]

In the general formulas (TEMP-34) to (TEMP-41), * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

### Substituted or Unsubstituted Arylene Group

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

### Substituted or Unsubstituted Divalent Heterocyclic Group

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

### Substituted or Unsubstituted Alkylene Group

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

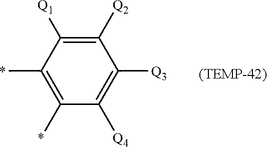

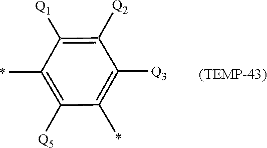

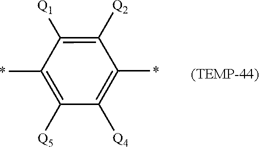

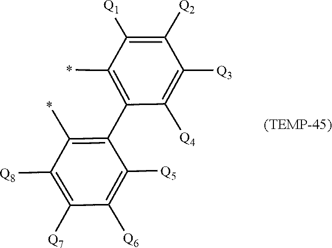

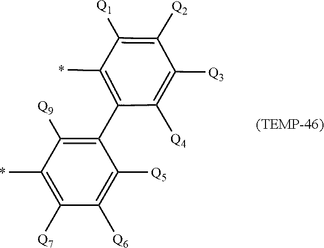

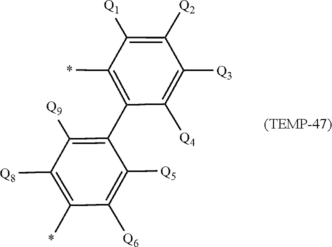

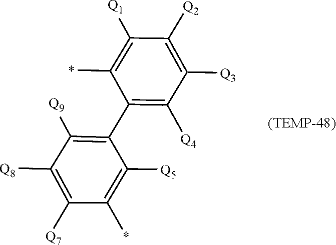

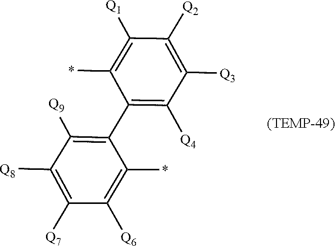

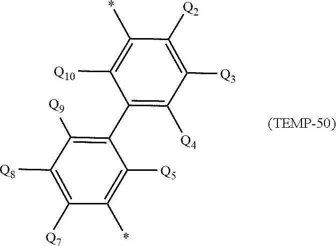

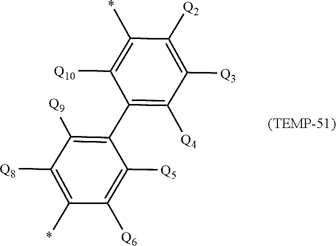

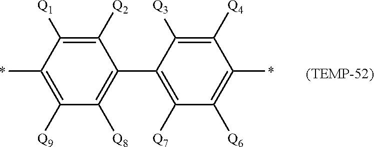

In the general formulas (TEMP-42) to (TEMP-52), Q₁ to Q₁₀ are independently a hydrogen atom or a substituent.

In the general formulas (TEMP-42) to (TEMP-52), * represents a bonding site.

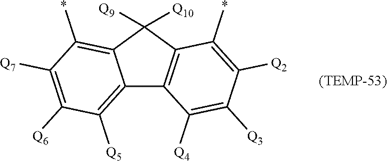

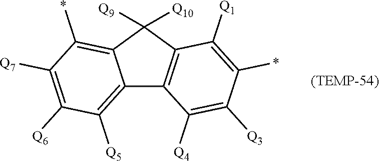

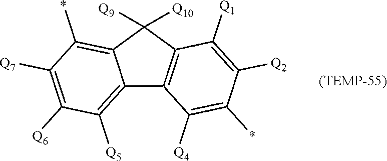

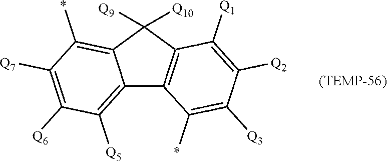

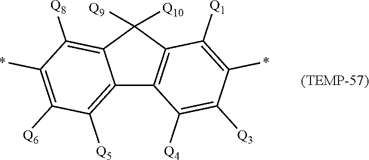

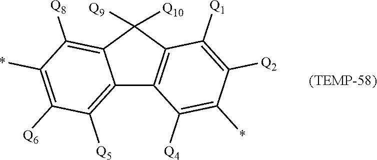

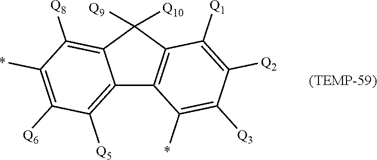

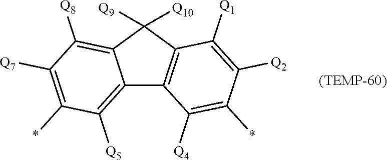

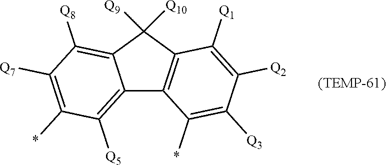

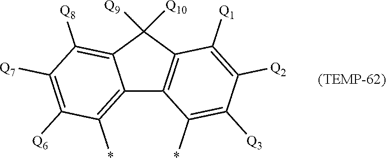

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding site.

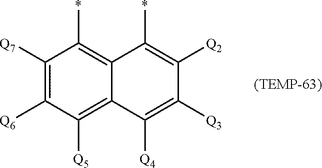

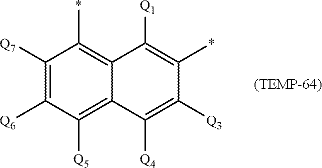

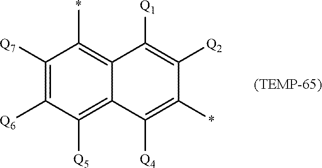

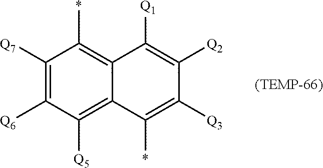

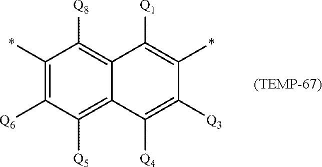

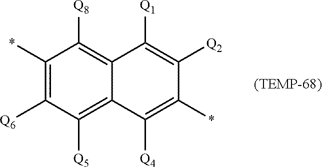

In the general formulas (TEMP-63) to (TEMP-68), Q₁ to Q₈ are independently a hydrogen atom or a substituent.

In the general formulas (TEMP-63) to (TEMP-68), * represents a bonding site.

The substituted or unsubstituted divalent heterocyclic group described in this specification is preferably any group of the following general formulas (TEMP-69) to (TEMP-102), unless otherwise specified in this specification.

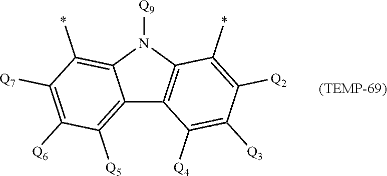

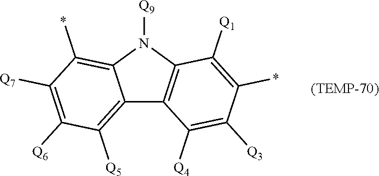

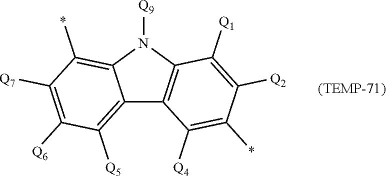

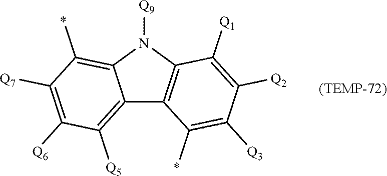

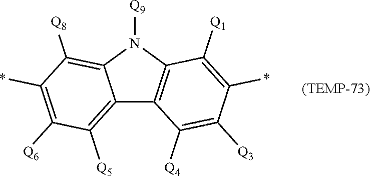

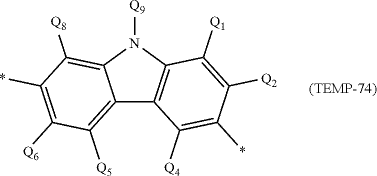

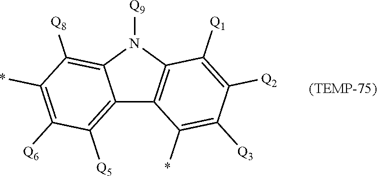

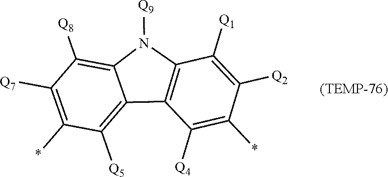

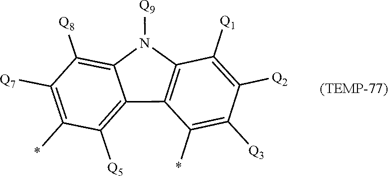

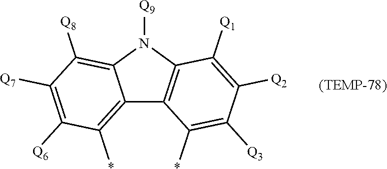

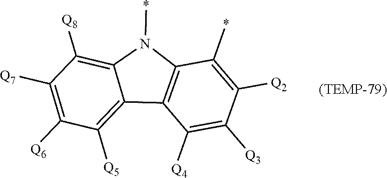

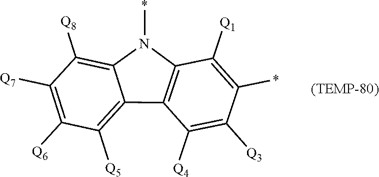

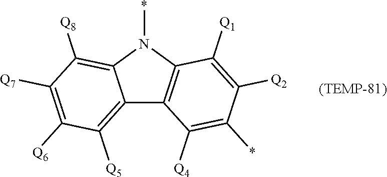

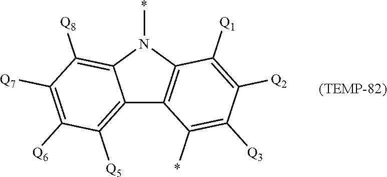

In the general formulas (TEMP-69) to (TEMP-82), Q₁ to Q₉ are independently a hydrogen atom or a substituent.

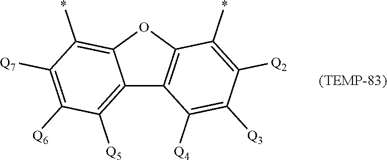

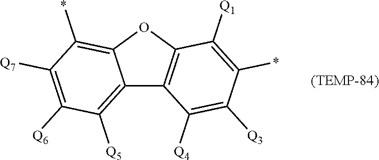

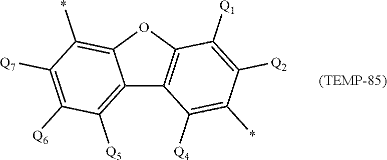

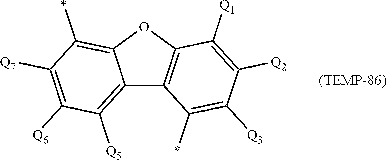

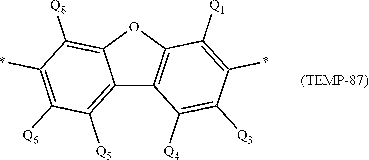

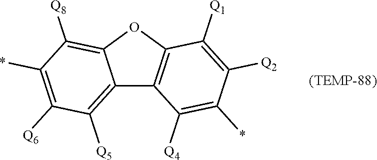

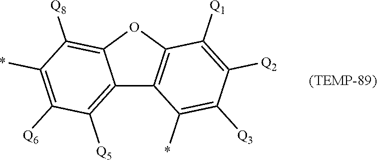

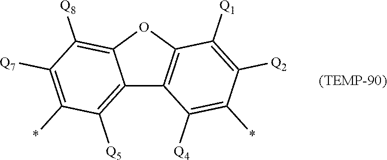

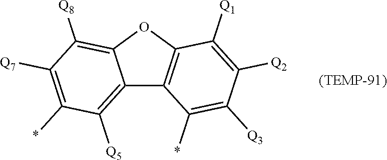

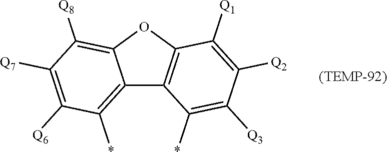

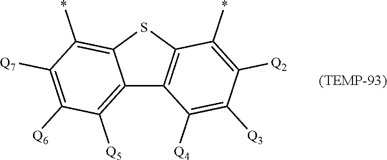

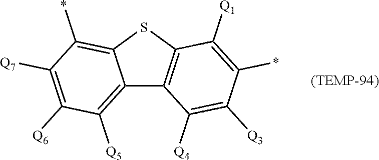

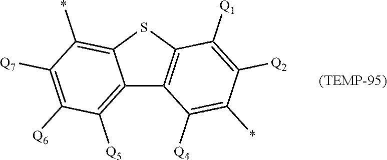

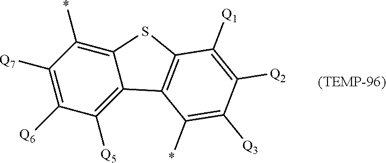

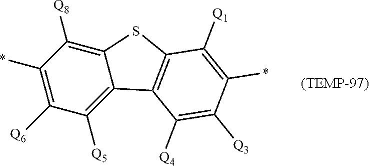

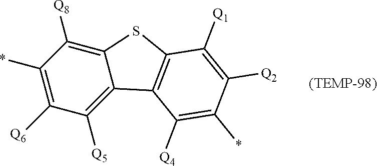

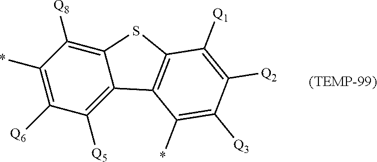

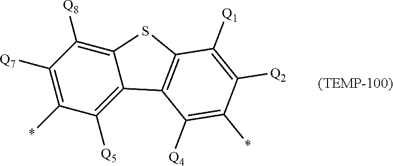

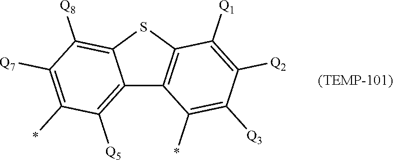

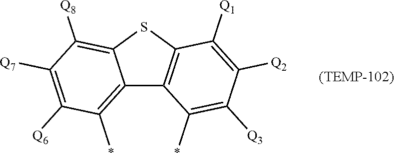

In the general formulas (TEMP-83) to (TEMP-102), Q₁ to Q₈ are independently a hydrogen atom or a substituent.

The above is the explanation of the “Substituent described in this specification.”

### The Case Where Bonded With Each Other to Form a Ring

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

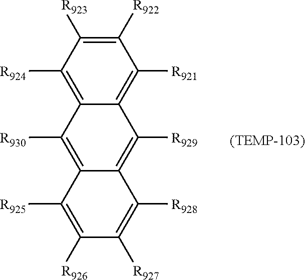

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

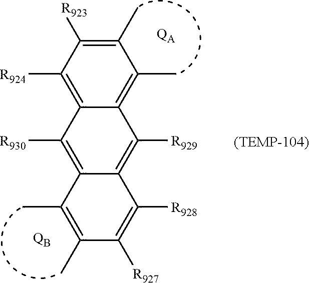

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

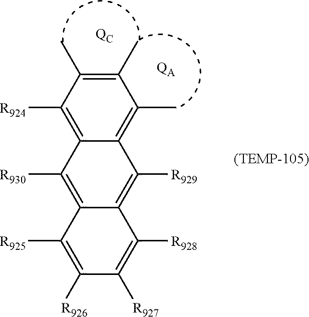

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and/or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

### Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.
When two or more R₉₀₁’s are present, the two or more R₉₀₁’s may be the same or different.
When two or more R₉₀₂’s are present, the two or more R₉₀₂’s may be the same or different.
When two or more R₉₀₃’s are present, the two or more R₉₀₃’s may be the same or different.
When two or more R₉₀₄’s are present, the two or more R₉₀₄’s may be the same or different.
When two or more R₉₀₅’s are present, the two or more R₉₀₅’s may be the same or different.
When two or more R₉₀₆’s are present, the two or more R₉₀₆’s may be the same or different.
When two or more R₉₀₇’s are present, the two or more R₉₀₇’s may be the same or different.

In one embodiment, the substituent in the case of “substituted or unsubstituted” is a group selected from the group consisting of:
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### Novel Compound

A compound according to an aspect of the present invention is represented by the following formula (1):

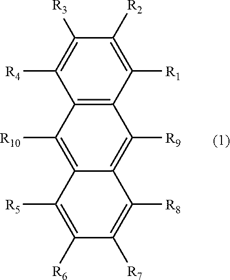

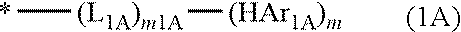

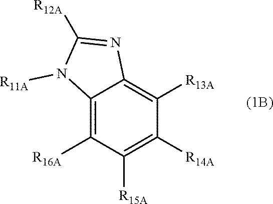

wherein in the formula (1),
at least one of R₁ to R₁₀ is a group represented by the formula (1A);
one or more sets of the adjacent two or more of R₁ to R₁₀ which are not the group represented by the formula (1A) form a substituted or unsubstituted, saturated or unsaturated ring by bonding with each other, or do not bond with each other;
R₁ to R₁₀ which are not the group represented by the formula (1A) and which do not bond with each other are independently a hydrogen atom or a substituent X;
the substituent X is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₅ are present, the two or more of each of R₉₀₁ to R₉₀₅ may be the same as or different from each other;
   when two or more substituents X are present, the two or more substituents X may be the same as or different from each other;
in the formula (1A),
HAr_{1A} is a group represented by the formula (1B); m is an integer of 1 to 5; when two or more HAr_{1A}’s are present, the two or more HAr_{1A}’s may be the same as or different from each other;
L_{1A} is
a substituted or unsubstituted aromatic hydrocarbon group having 6 to 30 ring carbon atoms, or
a substituted or unsubstituted heterocyclic group having 5 to 30 ring atoms;
m1A is an integer of 0 to 3; when m1A is 0, m is 1 and HAr_{1A} is directly bonded with the anthracene skeleton in the formula (1) via a single bond; when m1A is 2 or 3, a plurality of L_{1A}’s is linked in series with each other and HAr_{1A} is bonded with L_{1A} which is farthest from the anthracene skeleton; when two or more L_{1A}’s are present, the two or more L_{1A}’s may be the same as or different from each other;
one or more sets of the adjacent two or more of L_{1A}’s and m Ar_{1A}’s do not form a substituted or unsubstituted, saturated or unsaturated ring;
in the formula (1B),
any one of R_{11A} to R_{16A} represents a bond with L_{1A} in the formula (1A); when m1A is 0, any of carbon atoms and nitrogen atoms in the benzimidazole and any of carbon atoms in the anthracene of the formula (1) are directly bonded via a single bond;
R_{11A} to R_{16A} which do not represent a bond with L_{1A} are independently a hydrogen atom or a substituent Y; one or more sets of the adjacent two or more of R_{11A} to R_{16A} which do not represent a bond with L_{1A} do not bond with each other;
the substituent Y is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are the same as defined in the formula (1);
R₉₀₆ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₆ to R₉₀₇ are present, the two or more of each of R₉₀₆ to R₉₀₇ may be the same as or different from each other;
when two or more substituents Y are present, the two or more substituents Y may be the same as or different from each other;
provided that the compound represented by the formula (1) satisfies all of the following conditions 1 to 6;
condition 1: the compound represented by the formula (1) includes at least one deuterium atom;
condition 2: at least one of R₉ and R₁₀ is the group represented by the formula (1A) or the substituent X;
condition 3: when at least one of R₁ to R₈ in the formula (1) is the substituent X, m is 1 in the formula (1A) and the substituent in the case of “substituted or unsubstituted” wherein L_{1A} is a substituted or unsubstituted phenyl group is not a substituted or unsubstituted benzimidazolyl group;
condition 4: when at least one of R₁ to R₈ in the formula (1) is a substituted or unsubstituted naphthyl group, R_{12A} in the formula (1B) do not represent a bond with L_{1A} in the formula (1A);
condition 5: when at least one of R₂, R₃, R₆, and R₇ in the formula (1) is the group represented by the formula (1A), at least one hydrogen atom selected from the group consisting of, R₁ to R₁₀ which are hydrogen atoms, hydrogen atoms possessed by the substituted or unsubstituted, saturated or unsaturated ring formed in the case where one or more sets of the adjacent two or more of R₁ to R₁₀ bond with each other, hydrogen atoms possessed by R₁ to R₁₀ which are the substituents X, and hydrogen atoms possessed by L_{1A} is a deuterium atom; and
condition 6: when any one of R_{14A} and R_{15A} in the formula (1B) represents a bond with L_{1A} in the formula (1A), m1A is not 0.

When the compound according to an aspect of the present invention has the above structure, the compound is used in an organic EL device to be capable of enhancing the device performance thereof. Specifically, the compound according to an aspect of the present invention can prolong the lifetime of the organic EL device.

At least one of R₁ to R₁₀ in the formula (1) is the group represented by the formula (1A). The number of the group represented by the formula (1A) is not limited in R₁ to R₁₀, and only any one of R₁ to R₁₀ may be the group represented by the formula (1A), or two or more of R₁ to R₁₀ may be the group represented by the formula (1A). When a plurality of the groups represented by the formula (1A) is present, each of the groups represented by the formula (1A) may be the same as or different from each other.

In one embodiment, at least one of R₁, R₄, R₅, and R₈ to R₁₀ is the group represented by the formula (1A).

In one embodiment, at least one of R₉ and R₁₀ is the group represented by the formula (1A).

In one embodiment, at least one of R₉ and R₁₀ is the substituent X, or the group represented by the formula (1A).

The group represented by the formula (1A) will be described.

When m1A is 0, m is 1 and HAr_{1A} is directly bonded with the anthracene skeleton in the formula (1) via a single bond. When m1A is 2 or 3, a plurality of L_{1A}’s is linked in series with each other and HAr_{1A} is bonded with L_{1A} which is farthest from the anthracene skeleton.

When m is 2 or more and m1A is 1, the two or more of each of HAr_{1A}’s are bonded with L_{1A}. When m is 2 or more and m1A is 2 or 3, the two or more of each of HAr_{1A}’s are is bonded with L_{1A} which is farthest from the anthracene skeleton.

For example, when R₉ is the group represented by the formula (1A), m1A is 2, and m is 2, the compound represented by the formula (1) is the following structure.

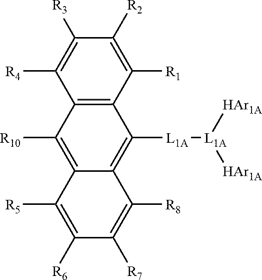

In one embodiment, L_{1A} is an unsubstituted aromatic hydrocarbon group having 6 to 30 ring carbon atoms, or an unsubstituted heterocyclic group having 5 to 30 ring atoms.

In one embodiment, L_{1A} is a substituted or unsubstituted aromatic hydrocarbon group having 6 to 30 ring carbon atoms.

m1A is not particularly limited, m1A may be any of 0, 1, 2 and 3, and in one embodiment, m1A is 1 or 2.

m is not particularly limited, m may be any of 1, 2, 3, 4 and 5, and in one embodiment, m is 1.

The group represented by the formula (1B) will be described.

Any one of R_{11A} to R_{16A} represents a bond with L_{1A} in the formula (1A). The expression “represents a bond” means any of carbon atoms and nitrogen atoms in the benzimidazole which R_{11A} to R_{16A} are bonded with and L_{1A} are directly bonded. When m1A is 0, any of carbon atoms and nitrogen atoms in the benzimidazole and any of carbon atoms in the anthracene of the formula (1) are directly bonded via a single bond.

In one embodiment, any one of R_{11A} to R_{13A} and R_{16A} represents a bonding position with L_{1A} in the formula (1A).

In one embodiment, any one of R_{11A} and R_{13A} to R_{16A} represents a bonding position with L_{1A} in the formula (1A).

In one embodiment, any one of R_{11A} and R_{13A} to R_{16A} represents a bond with L_{1A} in the formula (1A),
R_{12A} is
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are the same as defined in the formula (1)),
a halogen atom, a cyano group, a nitro group,
an unsubstituted aryl group having 6 to 50 ring carbon atoms, or
a substituted or unsubstituted monovalent heterocyclic group having 5 to 50 ring atoms.

In one embodiment, R_{11A} represents a bonding position with L_{1A} in the formula (1A).

R_{11A} to R_{16A} which do not represent a bond with L_{1A} are independently a hydrogen atom or a substituent Y. All of R_{11A} to R_{16A} which do not represent a bond with L_{1A} may be hydrogen atoms, or part of them may be the substituent Y, and for example, R_{11A} or R_{12A} may be the substituent Y.

When R_{11A} represents a bonding position with L_{1A}, R_{12A} may be the substituent Y. In such a case, R_{13A} to R_{16A} may be hydrogen atoms.

In one embodiment, R_{12A} represents a bonding position with L_{1A} in the formula (1A). In such a case, R_{11A} may be the substituent Y. In such a case, R_{13A} to R_{16A} may be hydrogen atoms.

In one embodiment, the substituent Y is a substituted or unsubstituted alkyl group having 1 to 50 carbon atoms, or a substituted or unsubstituted aryl group having 6 to 50 ring carbon atoms.

R₁ to R₁₀ which are not the group represented by the formula (1A) will be described.

In one embodiment, one or more sets of the adjacent two or more of R₁ to R₁₀ do not bond with each other.

R₁ to R₁₀ which are not the group represented by the formula (1A) are independently a hydrogen atom or a substituent X, and all of them may be hydrogen atoms, or part of them may be the substituent X.

In one embodiment, R₁ to R₈ are hydrogen atoms.

In one embodiment, one of R₉ and R₁₀ is the group represented by the formula (1A), the other of R₉ and R₁₀ is the substituent X, and R₁ to R₈ are hydrogen atoms.

In one embodiment, one of R₉ and R₁₀ is the group represented by the formula (1A), the other of R₉ and R₁₀ is the substituent X, R₂ or R₃ is the substituent X, and others of R₁ to R₁₀ are hydrogen atoms.

In one embodiment, the substituent X is a substituted or unsubstituted aryl group having 6 to 50 ring carbon atoms, or a substituted or unsubstituted monovalent heterocyclic group having 5 to 50 ring atoms.

Next, the conditions 1 to 6 which the compound represented by the formula (1) should satisfy will be described.

### Condition 1

The compound represented by the formula (1) includes at least one deuterium atom.

As described in [Definition], the “hydrogen atom” used in the present specification includes a protium atom, a deuterium atom, and a tritium atom. Accordingly, the “hydrogen atom” in the compound represented by the formula (1) may basically contain naturally derived deuterium atoms, but the compound represented by the formula (1) is intentionally introduced deuterium atoms into by using a deuterated compound as a part or all of raw material compounds, and the like. That is, the compound represented by the formula (1) is a compound in which at least one of hydrogen atoms included therein is a deuterium atom.

At least one hydrogen atom selected from the following hydrogen atoms in the compound represented by the formula (1) is a deuterium atom.
(1-1) Hydrogen atoms possessed by the substituted or unsubstituted, saturated or unsaturated ring formed in the case where one or more sets of the adjacent two or more of R₁ to R₁₀ which are not the group represented by the formula (1A) bond with each other (which include hydrogen atoms directly bonded with ring atoms in the ring, and hydrogen atoms possessed by the substituent in the case where the ring has the substituent)
(1-2) R₁ to R₁₀ which are hydrogen atoms in the case where R₁ to R₁₀ are not the group represented by the formula (1A) and do not bond with each other
(1-3) Hydrogen atoms possessed by R₁ to R₁₀ which are the substituents X in the case where R₁ to R₁₀ are not the group represented by the formula (1A) and do not bond with each other (which include hydrogen atoms possessed by the substituent in the case where the substituent X has the substituent)
(1-4) Hydrogen atoms possessed by L_{1A} (which include hydrogen atoms possessed by the substituent in the case where L_{1A} has the substituent)
(1-5) R_{11A} to R_{16A} which are hydrogen atoms in the case where R_{11A} to R_{16A} do not represent a bond with L_{1A}
(1-6) Hydrogen atoms possessed by R_{11A} to R_{16A} which are the substituents Y in the case where R_{11A} to R_{16A} do not represent a bond with L_{1A} (which include hydrogen atoms possessed by the substituent in the case where the substituent Y has the substituent)

Regarding the above (1-2), in one embodiment, at least one of R₁ to R₈ may be a deuterium atom, and all of R₁ to R₈ may be deuterium atoms.

Regarding the above (1-3), in one embodiment, at least one of hydrogen atoms possessed by R₁ to R₁₀ (for example, R₉ or R₁₀) which are the substituents X may be a deuterium atom, and all of hydrogen atoms possessed by R₁ to R₁₀ (for example, R₉ or R₁₀) which are the substituents X may be deuterium atoms.

Regarding the above (1-4), in one embodiment, at least one of hydrogen atoms possessed by L_{1A} may be a deuterium atom, and all of hydrogen atoms possessed by L_{1A} may be deuterium atoms.

Regarding the above (1-5), in one embodiment, at least one of R_{11A} to R_{16A} is a deuterium atom, and for example, R_{12A} is a deuterium atom.

Regarding the above (1-6), in one embodiment, at least one of hydrogen atoms possessed by R_{11A} to R_{16A} which are the substituents Y in the case where R_{11A} to R_{16A} do not represent a bond with L_{1A} is a deuterium atom, and for example, at least one of hydrogen atoms possessed by R_{12A} which is the substituent Y is a deuterium atom.

In one embodiment, at least one of (or all of) (1-2) “R₁ to R₁₀ which are hydrogen atoms in the case where R₁ to R₁₀ are not the group represented by the formula (1A) and do not bond with each other” is a deuterium atom, and the other hydrogen atoms included in the formula (1) are protium atoms.

In one embodiment, at least one of (or all of) (1-6) “hydrogen atoms possessed by R_{11A} to R_{16A} which are the substituents Y in the case where R_{11A} to R_{16A} do not represent a bond with L_{1A}” is a deuterium atom, and the other hydrogen atoms included in the formula (1) are protium atoms. In one embodiment, at least one of (or all of) hydrogen atoms possessed by R_{12A} which is the substituent Y is a deuterium atom, and the other hydrogen atoms included in the formula (1) are protium atoms.

In one embodiment, at least one of (or all of) (1-6) “hydrogen atoms possessed by R_{11A} to R_{16A} which are the substituents Y in the case where R_{11A} to R_{16A} do not represent a bond with L_{1A}” is a protium atom, and the other hydrogen atoms included in the formula (1) are deuterium atoms. In one embodiment, at least one of (or all of) hydrogen atoms possessed by R_{12A} which is the substituent Y is a protium atom, and the other hydrogen atoms included in the formula (1) are deuterium atoms.

In one embodiment, at least one of (or all of) (1-6) “hydrogen atoms possessed by R_{11A} to R_{16A} which are the substituents Y in the case where R_{11A} to R_{16A} do not represent a bond with L_{1A}” is a deuterium atom, at least one of (or all of) (1-3) “hydrogen atoms possessed by R₁ to R₁₀ which are the substituents X in the case where R₁ to R₁₀ are not the group represented by the formula (1A) and do not bond with each other” is a deuterium atom, and the other hydrogen atoms included in the formula (1) are protium atoms. In one embodiment, at least one of (or all of) hydrogen atoms possessed by R_{12A} which is the substituent Y is a deuterium atom, at least one of (or all of) hydrogen atoms possessed by R₁₀ which is the substituent X is a deuterium atom, and the other hydrogen atoms included in the formula (1) are protium atoms.

In one embodiment, all of hydrogen atoms included in the formula (1) are deuterium atoms.

A compound may generally include naturally derived deuterium atoms. The deuteration rate specified below is a value calculated such that naturally derived deuterium atoms and intentionally introduced deuterium atoms do not distinguish and both of them contribute the deuteration rate. Specifically, it is a value expressed in percentage in which the number of deuterium atoms in the compound is divided by the number of hydrogen atoms (which are counted without isotope distinction) in the compound.

In one embodiment, the deuteration rate of the compound is, for example, 1% or more, 3% or more, 5% or more, 10% or more, or 50% or more.

### Condition 2

At least one of R₉ and R₁₀ is the group represented by the formula (1A) or the substituent X. In such a case, R₁ to R₈ are independently a hydrogen atom, a substituent X, or the group represented by the formula (1A), and in one embodiment, R₁ to R₈ are hydrogen atoms.

### Condition 3

When at least one of R₁ to R₈ in the formula (1) is the substituent X, m is 1 in the formula (1A) and the substituent in the case of “substituted or unsubstituted” wherein L_{1A} is a substituted or unsubstituted phenyl group is not a substituted or unsubstituted benzimidazolyl group. In such a case, L_{1A} may be unsubstituted. Any substituent other than the “substituted or unsubstituted benzimidazolyl group” can be used as the substituent in the case where it has the substituent.

### Condition 4

When at least one of R₁ to Rs in the formula (1) is a substituted or unsubstituted naphthyl group (that is, when at least one of R₁ to Rs in the formula (1) is the substituent X and the substituent X is a substituted or unsubstituted naphthyl group), R_{12A} in the formula (1B) do not represent a bond with L_{1A} in the formula (1A). In such a case, any one of R_{11A} and R_{13A} to R_{16A} represents a bond with L_{1A} in the formula (1A), and in one embodiment, R_{11A} represents a bond with L_{1A} in the formula (1A).

### Condition 5

When at least one of R₂, R₃, R₆, and R₇ in the formula (1) is the group represented by the formula (1A), at least one hydrogen atom selected from the group consisting of, R₁ to R₁₀ which are hydrogen atoms, hydrogen atoms possessed by the substituted or unsubstituted, saturated or unsaturated ring formed in the case where one or more sets of the adjacent two or more of R₁ to R₁₀ bond with each other (which include hydrogen atoms directly bonded with ring atoms in the ring, and hydrogen atoms possessed by the substituent in the case where the ring has the substituent), hydrogen atoms possessed by R₁ to R₁₀ which are the substituents X (which include hydrogen atoms possessed by the substituent in the case where the substituent X has the substituent), and hydrogen atoms possessed by L_{1A} (which include hydrogen atoms possessed by the substituent in the case where L_{1A} has the substituent) is a deuterium atom. In such a case, regarding at least one hydrogen atom selected from the group consisting of, hydrogen atoms possessed by the group represented by the formula (1B), that is, R_{11A} to R_{16A} which are hydrogen atoms in the case where R_{11A} to R_{16A} do not represent a bond with L_{1A}, and hydrogen atoms possessed by R_{11A} to R_{16A} which are the substituents Y in the case where R_{11A} to R_{16A} do not represent a bond with L_{1A} (which include hydrogen atoms possessed by the substituent in the case where the substituent Y has the substituent), all of them may be protium atoms, or part or all of them may be deuterium atoms.

### Condition 6

When any one of R_{14A} and R_{15A} in the formula (1B) represents a bond with L_{1A} in the formula (1A), m1A is not 0. In such a case, m1A is 1 or 2.

In one embodiment, the compound represented by the formula (1) is the compound represented by the following formula (11):

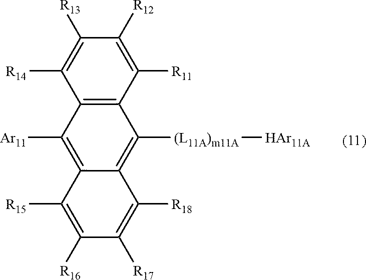

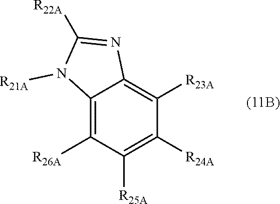

wherein in the formula (11),
one or more sets of the adjacent two or more of R₁₁ to R₁₈ form a substituted or unsubstituted, saturated or unsaturated ring by bonding with each other, or do not bond with each other;
R₁₁ to R₁₈ which do not bond with each other are independently a hydrogen atom or a substituent X;
the substituent X is the same as defined in the formula (1);
HAr_{11A} is a group represented by the formula (11B);
L_{11A} is
a substituted or unsubstituted aromatic hydrocarbon group having 6 to 30 ring carbon atoms, or
a substituted or unsubstituted heterocyclic group having 5 to 30 ring atoms;
m11A is an integer of 0 to 3; when m11A is 0, HAr_{11A} is directly bonded with the anthracene skeleton in the formula (11) via a single bond; when m11A is 2 or 3, a plurality of L_{11A}’s is linked in series with each other and HAr_{11A} is bonded with L_{11A} which is farthest from the anthracene skeleton; when two or more L_{11A}’s are present, the two or more L_{11A}’s may be the same as or different from each other;
one or more sets of the adjacent two or more of m11A L_{11A}’s and HAr_{11A} do not form a substituted or unsubstituted, saturated or unsaturated ring;
in the formula (11B),
any one of R_{21A} to R_{26A} represents a bond with L_{11A} in the formula (11); when m11A is 0, any of carbon atoms and nitrogen atoms in the benzimidazole and any of carbon atoms in the anthracene of the formula (11) are directly bonded via a single bond;
R_{21A} to R_{26A} which do not represent a bond with L_{11A} are independently a hydrogen atom or a substituent Y; one or more sets of the adjacent two or more of R_{21A} to R_{26A} do not bond with each other;
the substituent Y is the same as defined in the formula (1B);
Ar₁₁ is
a substituted or unsubstituted aryl group having 6 to 30 ring carbon atoms, or
a substituted or unsubstituted monovalent heterocyclic group having 5 to 30 ring atoms;
provided that the compound represented by the formula (11) includes at least one deuterium atom; when at least one of R₁₁ to R₁₈ is the substituent X, the substituent in the case of “substituted or unsubstituted” wherein L_{11A} is a substituted or unsubstituted phenyl group is not a substituted or unsubstituted benzimidazolyl group.

In one embodiment, one or more sets of the adjacent two or more of R₁₁ to R₁₈ do not bond with each other.

R₁₁ to R₁₈ are independently a hydrogen atom or a substituent X, and all of them may be hydrogen atoms, or part of them may be the substituent X.

In one embodiment, R₁₁ to R₁₈ are hydrogen atoms.

In one embodiment, R₁₂ or R₁₃ is the substituent X, and others of R₁₁ to R₁₈ are hydrogen atoms.

When m11A is 0, HAr_{11A} is directly bonded with the anthracene skeleton in the formula (11) via a single bond. When m11A is 2 or 3, a plurality of L_{11A}’s is linked in series with each other and HAr_{11A} is bonded with L_{11A} which is farthest from the anthracene skeleton.

In one embodiment, L_{11A} is a substituted or unsubstituted aromatic hydrocarbon group having 6 to 30 ring carbon atoms.

m11A is not particularly limited, m11A may be any of 0, 1, 2 and 3, and in one embodiment, m1A is 1 or 2.

R_{21A} to R_{26A} in the formula (11B) is the same as described in R_{11A} to R_{16A} of the formula (1B).

The compound represented by the formula (11) includes at least one deuterium atom. This is the same as described in the condition 1 of the formula (1), and specifically, at least one hydrogen atom selected from the following hydrogen atoms in the compound represented by the formula (11) is a deuterium atom.
(11-1) Hydrogen atoms possessed by the substituted or unsubstituted, saturated or unsaturated ring formed in the case where one or more sets of the adjacent two or more of R₁₁ to R₁₈ bond with each other (which include hydrogen atoms directly bonded with ring atoms in the ring, and hydrogen atoms possessed by the substituent in the case where the ring has the substituent)
(11-2) R₁₁ to R₁₈ which are hydrogen atoms in the case where R₁₁ to R₁₈ do not bond with each other
(11-3) Hydrogen atoms possessed by R₁₁ to R₁₈ which are the substituents X in the case where R₁₁ to R₁₈ do not bond with each other (which include hydrogen atoms possessed by the substituent in the case where the substituent X has the substituent)
(11-4) Hydrogen atoms possessed by Ar₁₁ (which include hydrogen atoms possessed by the substituent in the case where Ar₁₁ has the substituent)
(11-5) Hydrogen atoms possessed by L_{11A} (which include hydrogen atoms possessed by the substituent in the case where L_{11A} has the substituent)
(11-6) R_{21A} to R_{26A} which are hydrogen atoms in the case where R_{21A} to R_{26A} do not represent a bond with L_{11A}
(11-7) Hydrogen atoms possessed by R_{21A} to R_{26A} which are the substituents Y in the case where R_{21A} to R_{26A} do not represent a bond with L_{11A} (which include hydrogen atoms possessed by the substituent in the case where the substituent Y has the substituent)

Further, when at least one of R₁₁ to R₁₈ is the substituent X, the substituent in the case of “substituted or unsubstituted” wherein L_{11A} is a substituted or unsubstituted phenyl group is not a substituted or unsubstituted benzimidazolyl group. This is corresponding to the condition 3 in the formula (1), and when at least one of R₁₁ to R₁₈ is the substituent X, L_{11A} may be unsubstituted. Any substituent other than the “substituted or unsubstituted benzimidazolyl group” can be used as the substituent in the case where it has the substituent.

In one embodiment, the compound represented by the formula (11) is the compound represented by the following formula (12):

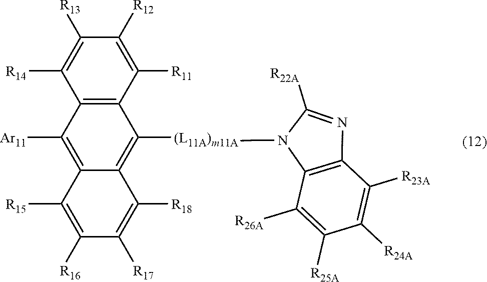

wherein in the formula (12),
R₁₁ to R₁₈, L_{11A}, m11A, and Ar₁₁ are the same as defined in the formula (11);
R_{22A} to R_{26A} are independently a hydrogen atom or a substituent Y; one or more sets of the adjacent two or more of R_{22A} to R_{26A} do not bond with each other;
the substituent Y is the same as defined in the formula (1B);
provided that the compound represented by the formula (12) includes at least one deuterium atom; when at least one of R₁₁ to R₁₈ is the substituent X, the substituent in the case of “substituted or unsubstituted” wherein L_{11A} is a substituted or unsubstituted phenyl group is not a substituted or unsubstituted benzimidazolyl group.

The compound represented by the formula (12) is one in which the bonding position of the group represented by the formula (11B) and L_{11A} is specified in the compound represented by the formula (11). The conditions other than that are the same as described in the formula (11).

In one embodiment, m11A is 1 in the formula (11) or the formula (12).

In one embodiment, the compound represented by the formula (11) is the compound represented by the following formula (13):

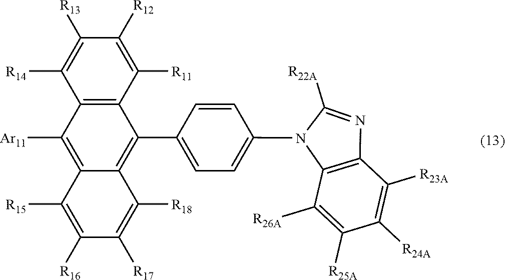

wherein in the formula (13),
R₁₁ to R₁₈, R_{22A} to R_{26A}, and Ar₁₁ are the same as defined in the formula (11);
provided that the compound represented by the formula (13) includes at least one deuterium atom.

The compound represented by the formula (13) is one in which aspects of L_{11A} and m11A are specified in the compound represented by the formula (12). The conditions other than that are the same as described in the formula (11) or (12).

As understood by [Definition], hydrogen atoms possessed by the phenylene group arranged between the anthracene skeleton and the benzimidazole skeleton may be deuterium atoms.

In one embodiment, the compound represented by the formula (11) is the compound represented by the following formula (14):

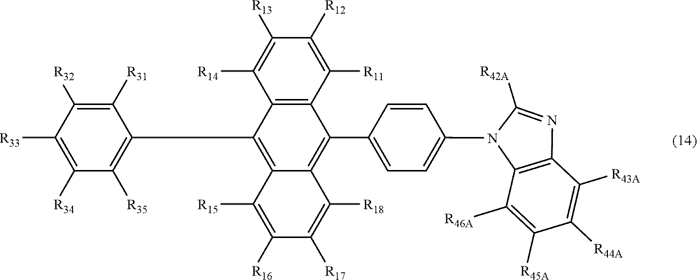

wherein in the formula (14),
R₁₁ to R₁₈ are the same as defined in the formula (11);
one or more sets of the adjacent two or more of R₃₁ to R₃₅ form a substituted or unsubstituted, saturated or unsaturated ring by bonding with each other, or do not bond with each other;
R₃₁ to R₃₅ which do not bond with each other are independently
a hydrogen atom,
a substituted or unsubstituted alkyl group having 1 to 50 carbon atoms,
a substituted or unsubstituted aryl group having 6 to 50 ring carbon atoms, or
a substituted or unsubstituted monovalent heterocyclic group having 5 to 50 ring atoms;
R_{42A} to R_{46A} are independently
a hydrogen atom, or
a substituted or unsubstituted alkyl group having 1 to 50 carbon atoms;
provided that the compound represented by the formula (14) includes at least one deuterium atom.

The compound represented by the formula (14) is one in which aspects of Ar₁₁ and R_{22A} to R_{26A} are specified in the compound represented by the formula (13).

In one embodiment, one or more sets of the adjacent two or more of R₃₁ to R₃₅ do not bond with each other, and R₃₁ to R₃₅ are independently a hydrogen atom, or a substituted or unsubstituted aryl group having 6 to 50 ring carbon atoms.

The conditions other than that are the same as described in the formulas (11) to (13).

As understood by [Definition], hydrogen atoms possessed by the phenylene group aranged between the anthracene skeleton and the benzimidazole skeleton may be deuterium atoms.

In one embodiment, the compound represented by the formula (14) is the compound represented by the following formula (15):

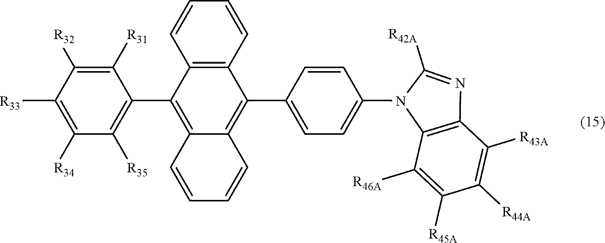

wherein in the formula (15),
R₃₁ to R₃₅ and R_{42A} to R_{46A} are the same as defined in the formula (14);
provided that the compound represented by the formula (15) comprises at least one deuterium atom.

The compound represented by the formula (15) is one in which aspects of R₁₁ to R₁₈ are specified in the compound represented by the formula (14). The conditions other than that are the same as described in the formulas (11) to (14).

As understood by [Definition], hydrogen atoms possessed by the phenylene group arranged between the anthracene skeleton and the benzimidazole skeleton may be deuterium atoms. Similarly, as understood by [Definition], hydrogen atoms possessed by the anthracene skeleton may be deuterium atoms.

In one embodiment, the substituent in the case of “substituted or unsubstituted”, the substituent X, and the substituent Y are a group selected from the group consisting of
an alkyl group having 1 to 50 carbon atoms,
an aryl group having 6 to 50 ring carbon atoms, and
a monovalent heterocyclic group having 5 to 50 ring atoms.

In one embodiment, the substituent in the case of “substituted or unsubstituted”, the substituent X, and the substituent Y are a group selected from the group consisting of
an alkyl group having 1 to 18 carbon atoms,
an aryl group having 6 to 18 ring carbon atoms, and
a monovalent heterocyclic group having 5 to 18 ring atoms.

The compound according to an aspect of the present invention (the compound represented by the formula (1)) can be synthesized in accordance with Examples by using known alternative reactions or raw materials adapted to the target compound.

Specific examples of the compound according to an aspect of the present invention (the compound represented by the formula (1)) will be described below, but these are merely examples, and the compound according to an aspect of the present invention is not limited to the following specific examples.

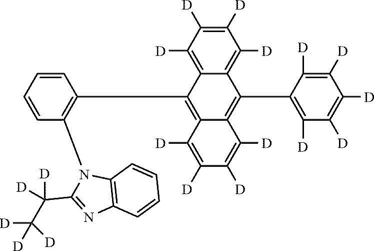 [SMILES: [2H]c1c([2H])c([2H])c(-c2c3c([2H])c([2H])c([2H])c([2H])c3c(-c3ccccc3-n3c(C([2H])([2H])C([2H])([2H])[2H])nc4ccccc43)c3c([2H])c([2H])c([2H])c([2H])c23)c([2H])c1[2H]]

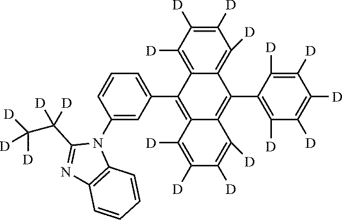 [SMILES: [2H]c1c([2H])c([2H])c(-c2c3c([2H])c([2H])c([2H])c([2H])c3c(-c3cccc(-n4c(C([2H])([2H])C([2H])([2H])[2H])nc5ccccc54)c3)c3c([2H])c([2H])c([2H])c([2H])c23)c([2H])c1[2H]]

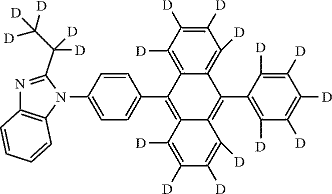 [SMILES: [2H]c1c([2H])c([2H])c(-c2c3c([2H])c([2H])c([2H])c([2H])c3c(-c3ccc(-n4c(C([2H])([2H])C([2H])([2H])[2H])nc5ccccc54)cc3)c3c([2H])c([2H])c([2H])c([2H])c23)c([2H])c1[2H]]

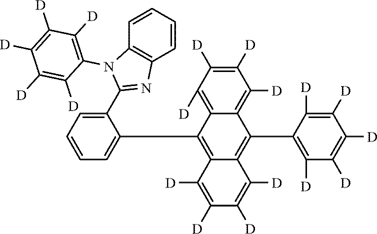 [SMILES: [2H]c1c([2H])c([2H])c(-c2c3c([2H])c([2H])c([2H])c([2H])c3c(-c3ccccc3-c3nc4ccccc4n3-c3c([2H])c([2H])c([2H])c([2H])c3[2H])c3c([2H])c([2H])c([2H])c([2H])c23)c([2H])c1[2H]]

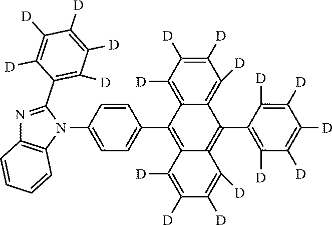 [SMILES: [2H]c1c([2H])c([2H])c(-c2c3c([2H])c([2H])c([2H])c([2H])c3c(-c3ccc(-n4c(-c5c([2H])c([2H])c([2H])c([2H])c5[2H])nc5ccccc54)cc3)c3c([2H])c([2H])c([2H])c([2H])c23)c([2H])c1[2H]]

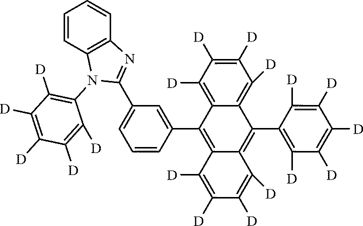 [SMILES: [2H]c1c([2H])c([2H])c(-c2c3c([2H])c([2H])c([2H])c([2H])c3c(-c3cccc(-c4nc5ccccc5n4-c4c([2H])c([2H])c([2H])c([2H])c4[2H])c3)c3c([2H])c([2H])c([2H])c([2H])c23)c([2H])c1[2H]]

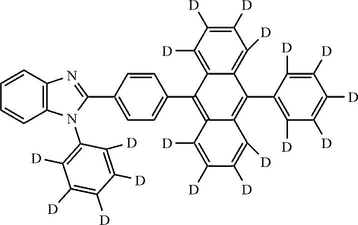 [SMILES: [2H]c1c([2H])c([2H])c(-c2c3c([2H])c([2H])c([2H])c([2H])c3c(-c3ccc(-c4nc5ccccc5n4-c4c([2H])c([2H])c([2H])c([2H])c4[2H])cc3)c3c([2H])c([2H])c([2H])c([2H])c23)c([2H])c1[2H]]

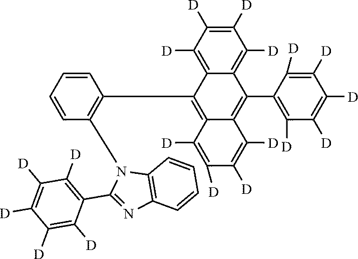 [SMILES: [2H]c1c([2H])c([2H])c(-c2c3c([2H])c([2H])c([2H])c([2H])c3c(-c3ccccc3-n3c(-c4c([2H])c([2H])c([2H])c([2H])c4[2H])nc4ccccc43)c3c([2H])c([2H])c([2H])c([2H])c23)c([2H])c1[2H]]

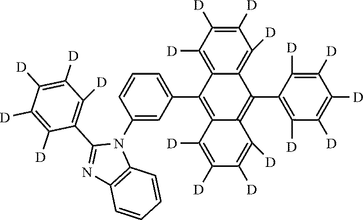 [SMILES: [2H]c1c([2H])c([2H])c(-c2c3c([2H])c([2H])c([2H])c([2H])c3c(-c3cccc(-n4c(-c5c([2H])c([2H])c([2H])c([2H])c5[2H])nc5ccccc54)c3)c3c([2H])c([2H])c([2H])c([2H])c23)c([2H])c1[2H]]

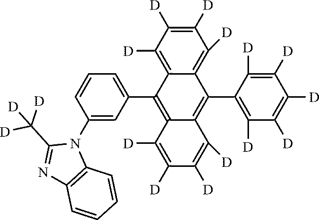 [SMILES: [2H]c1c([2H])c([2H])c(-c2c3c([2H])c([2H])c([2H])c([2H])c3c(-c3cccc(-n4c(C([2H])([2H])[2H])nc5ccccc54)c3)c3c([2H])c([2H])c([2H])c([2H])c23)c([2H])c1[2H]]

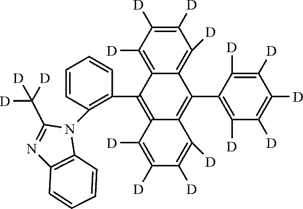 [SMILES: [2H]c1c([2H])c([2H])c(-c2c3c([2H])c([2H])c([2H])c([2H])c3c(-c3ccccc3-n3c(C([2H])([2H])[2H])nc4ccccc43)c3c([2H])c([2H])c([2H])c([2H])c23)c([2H])c1[2H]]

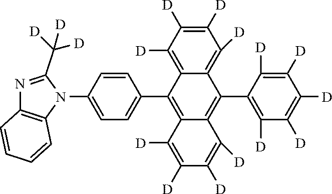 [SMILES: [2H]c1c([2H])c([2H])c(-c2c3c([2H])c([2H])c([2H])c([2H])c3c(-c3ccc(-n4c(C([2H])([2H])[2H])nc5ccccc54)cc3)c3c([2H])c([2H])c([2H])c([2H])c23)c([2H])c1[2H]]

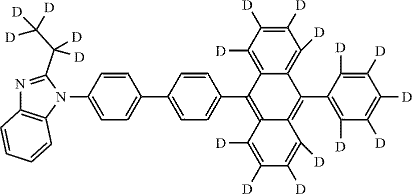 [SMILES: [2H]c1c([2H])c([2H])c(-c2c3c([2H])c([2H])c([2H])c([2H])c3c(-c3ccc(-c4ccc(-n5c(C([2H])([2H])C([2H])([2H])[2H])nc6ccccc65)cc4)cc3)c3c([2H])c([2H])c([2H])c([2H])c23)c([2H])c1[2H]]

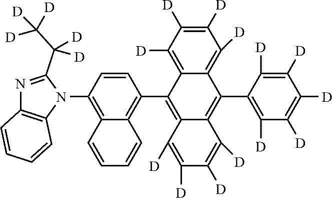 [SMILES: [2H]c1c([2H])c([2H])c(-c2c3c([2H])c([2H])c([2H])c([2H])c3c(-c3ccc(-n4c(C([2H])([2H])C([2H])([2H])[2H])nc5ccccc54)c4ccccc34)c3c([2H])c([2H])c([2H])c([2H])c23)c([2H])c1[2H]]

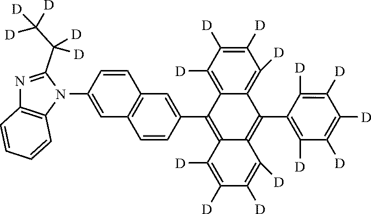 [SMILES: [2H]c1c([2H])c([2H])c(-c2c3c([2H])c([2H])c([2H])c([2H])c3c(-c3ccc4cc(-n5c(C([2H])([2H])C([2H])([2H])[2H])nc6ccccc65)ccc4c3)c3c([2H])c([2H])c([2H])c([2H])c23)c([2H])c1[2H]]

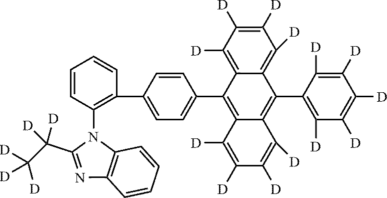 [SMILES: [2H]c1c([2H])c([2H])c(-c2c3c([2H])c([2H])c([2H])c([2H])c3c(-c3ccc(-c4ccccc4-n4c(C([2H])([2H])C([2H])([2H])[2H])nc5ccccc54)cc3)c3c([2H])c([2H])c([2H])c([2H])c23)c([2H])c1[2H]]

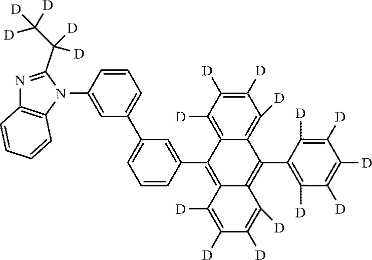 [SMILES: [2H]c1c([2H])c([2H])c(-c2c3c([2H])c([2H])c([2H])c([2H])c3c(-c3cccc(-c4cccc(-n5c(C([2H])([2H])C([2H])([2H])[2H])nc6ccccc65)c4)c3)c3c([2H])c([2H])c([2H])c([2H])c23)c([2H])c1[2H]]

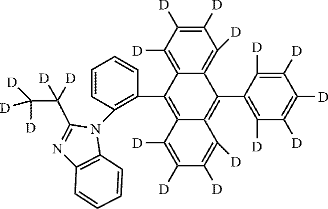 [SMILES: [2H]c1c([2H])c([2H])c(-c2c3c([2H])c([2H])c([2H])c([2H])c3c(-c3ccccc3-n3c(C([2H])([2H])C([2H])([2H])[2H])nc4ccccc43)c3c([2H])c([2H])c([2H])c([2H])c23)c([2H])c1[2H]]

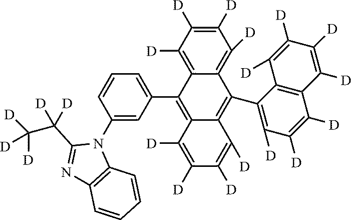 [SMILES: [2H]c1c([2H])c([2H])c2c(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)c([2H])c([2H])c([2H])c2c1[2H]]

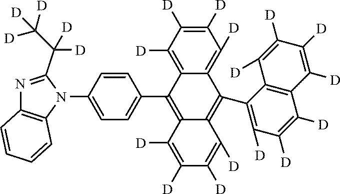 [SMILES: [2H]c1c([2H])c([2H])c2c(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)c([2H])c([2H])c([2H])c2c1[2H]]

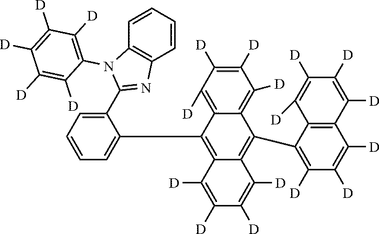 [SMILES: [2H]c1c([2H])c([2H])c(-n2c(-c3ccccc3-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)nc3ccccc32)c([2H])c1[2H]]

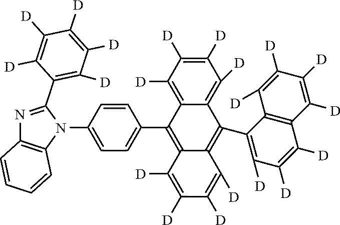 [SMILES: [2H]c1c([2H])c([2H])c(-c2nc3ccccc3n2-c2ccc(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)cc2)c([2H])c1[2H]]

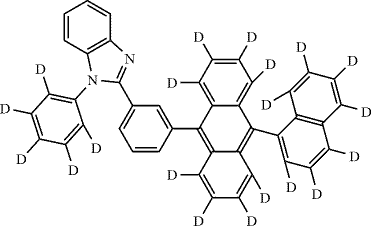 [SMILES: [2H]c1c([2H])c([2H])c(-n2c(-c3cccc(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)c3)nc3ccccc32)c([2H])c1[2H]]

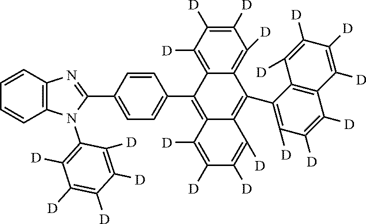 [SMILES: [2H]c1c([2H])c([2H])c(-n2c(-c3ccc(-c4c5c([2H])c([2H])c([2H])c([2H])c5c(-c5c([2H])c([2H])c([2H])c6c([2H])c([2H])c([2H])c([2H])c56)c5c([2H])c([2H])c([2H])c([2H])c45)cc3)nc3ccccc32)c([2H])c1[2H]]

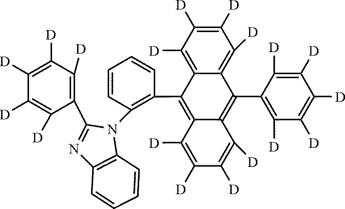 [SMILES: [2H]c1c([2H])c([2H])c(-c2c3c([2H])c([2H])c([2H])c([2H])c3c(-c3ccccc3-n3c(-c4c([2H])c([2H])c([2H])c([2H])c4[2H])nc4ccccc43)c3c([2H])c([2H])c([2H])c([2H])c23)c([2H])c1[2H]]

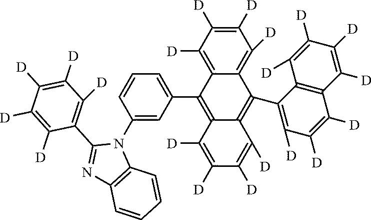 [SMILES: [2H]c1c([2H])c([2H])c(-c2nc3ccccc3n2-c2cccc(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)c2)c([2H])c1[2H]]

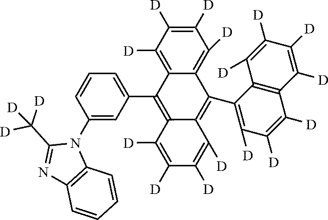 [SMILES: [2H]c1c([2H])c([2H])c2c(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)c([2H])c([2H])c([2H])c2c1[2H]]

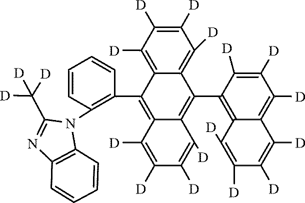 [SMILES: [2H]c1c([2H])c([2H])c2c(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)c([2H])c([2H])c([2H])c2c1[2H]]

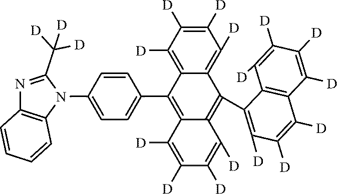 [SMILES: [2H]c1c([2H])c([2H])c2c(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)c([2H])c([2H])c([2H])c2c1[2H]]

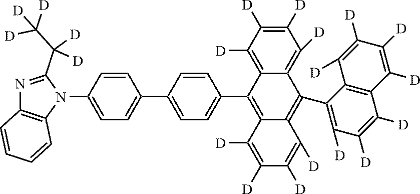 [SMILES: [2H]c1c([2H])c([2H])c2c(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)c([2H])c([2H])c([2H])c2c1[2H]]

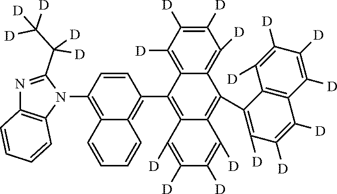 [SMILES: [2H]c1c([2H])c([2H])c2c(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)c([2H])c([2H])c([2H])c2c1[2H]]

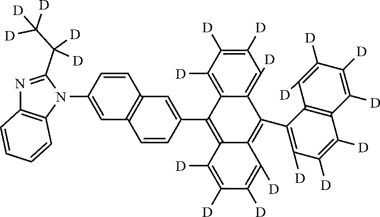 [SMILES: [2H]c1c([2H])c([2H])c2c(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)c([2H])c([2H])c([2H])c2c1[2H]]

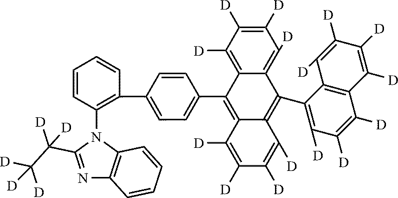 [SMILES: [2H]c1c([2H])c([2H])c2c(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)c([2H])c([2H])c([2H])c2c1[2H]]

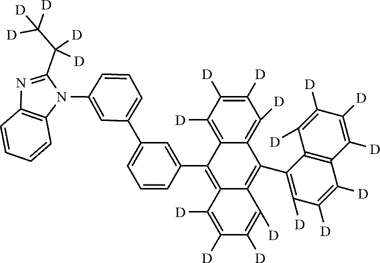 [SMILES: [2H]c1c([2H])c([2H])c2c(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)c([2H])c([2H])c([2H])c2c1[2H]]

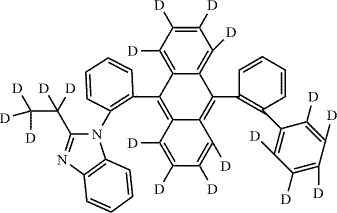 [SMILES: [2H]c1c([2H])c([2H])c(-c2ccccc2-c2c3c([2H])c([2H])c([2H])c([2H])c3c(-c3ccccc3-n3c(C([2H])([2H])C([2H])([2H])[2H])nc4ccccc43)c3c([2H])c([2H])c([2H])c([2H])c23)c([2H])c1[2H]]

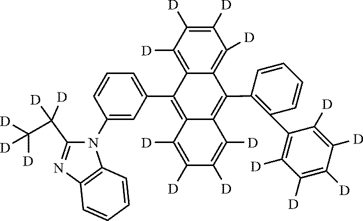 [SMILES: [2H]c1c([2H])c([2H])c(-c2ccccc2-c2c3c([2H])c([2H])c([2H])c([2H])c3c(-c3cccc(-n4c(C([2H])([2H])C([2H])([2H])[2H])nc5ccccc54)c3)c3c([2H])c([2H])c([2H])c([2H])c23)c([2H])c1[2H]]

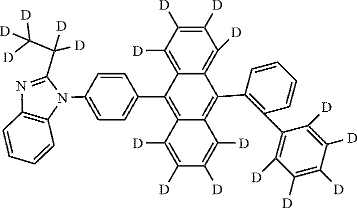 [SMILES: [2H]c1c([2H])c([2H])c(-c2ccccc2-c2c3c([2H])c([2H])c([2H])c([2H])c3c(-c3ccc(-n4c(C([2H])([2H])C([2H])([2H])[2H])nc5ccccc54)cc3)c3c([2H])c([2H])c([2H])c([2H])c23)c([2H])c1[2H]]

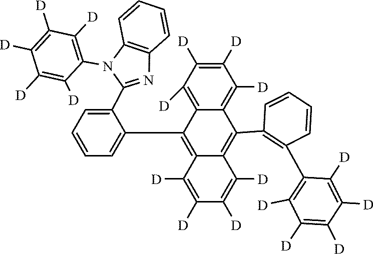 [SMILES: [2H]c1c([2H])c([2H])c(-c2ccccc2-c2c3c([2H])c([2H])c([2H])c([2H])c3c(-c3ccccc3-c3nc4ccccc4n3-c3c([2H])c([2H])c([2H])c([2H])c3[2H])c3c([2H])c([2H])c([2H])c([2H])c23)c([2H])c1[2H]]

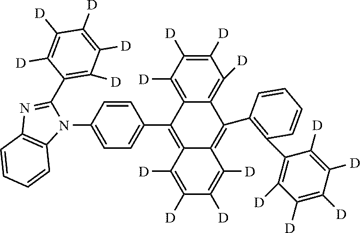 [SMILES: [2H]c1c([2H])c([2H])c(-c2ccccc2-c2c3c([2H])c([2H])c([2H])c([2H])c3c(-c3ccc(-n4c(-c5c([2H])c([2H])c([2H])c([2H])c5[2H])nc5ccccc54)cc3)c3c([2H])c([2H])c([2H])c([2H])c23)c([2H])c1[2H]]

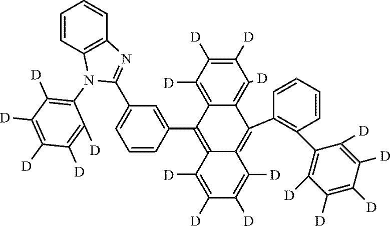 [SMILES: [2H]c1c([2H])c([2H])c(-c2ccccc2-c2c3c([2H])c([2H])c([2H])c([2H])c3c(-c3cccc(-c4nc5ccccc5n4-c4c([2H])c([2H])c([2H])c([2H])c4[2H])c3)c3c([2H])c([2H])c([2H])c([2H])c23)c([2H])c1[2H]]

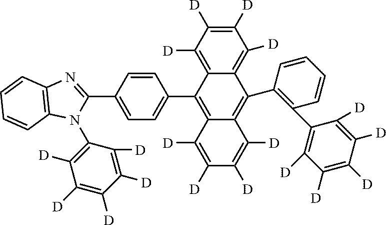 [SMILES: [2H]c1c([2H])c([2H])c(-c2ccccc2-c2c3c([2H])c([2H])c([2H])c([2H])c3c(-c3ccc(-c4nc5ccccc5n4-c4c([2H])c([2H])c([2H])c([2H])c4[2H])cc3)c3c([2H])c([2H])c([2H])c([2H])c23)c([2H])c1[2H]]

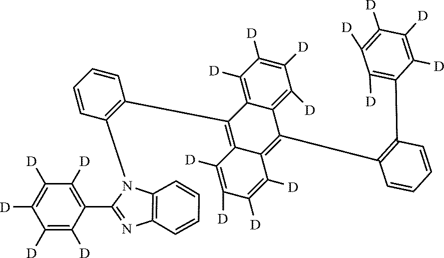 [SMILES: [2H]c1c([2H])c([2H])c(-c2ccccc2-c2c3c([2H])c([2H])c([2H])c([2H])c3c(-c3ccccc3-n3c(-c4c([2H])c([2H])c([2H])c([2H])c4[2H])nc4ccccc43)c3c([2H])c([2H])c([2H])c([2H])c23)c([2H])c1[2H]]

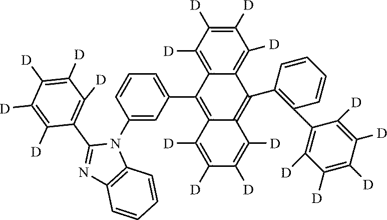 [SMILES: [2H]c1c([2H])c([2H])c(-c2ccccc2-c2c3c([2H])c([2H])c([2H])c([2H])c3c(-c3cccc(-n4c(-c5c([2H])c([2H])c([2H])c([2H])c5[2H])nc5ccccc54)c3)c3c([2H])c([2H])c([2H])c([2H])c23)c([2H])c1[2H]]

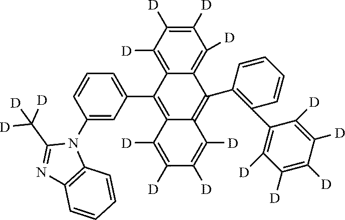 [SMILES: [2H]c1c([2H])c([2H])c(-c2ccccc2-c2c3c([2H])c([2H])c([2H])c([2H])c3c(-c3cccc(-n4c(C([2H])([2H])[2H])nc5ccccc54)c3)c3c([2H])c([2H])c([2H])c([2H])c23)c([2H])c1[2H]]

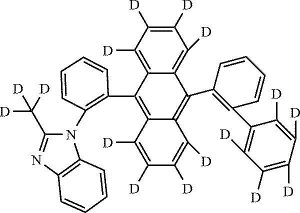 [SMILES: [2H]c1c([2H])c([2H])c(-c2ccccc2-c2c3c([2H])c([2H])c([2H])c([2H])c3c(-c3ccccc3-n3c(C([2H])([2H])[2H])nc4ccccc43)c3c([2H])c([2H])c([2H])c([2H])c23)c([2H])c1[2H]]

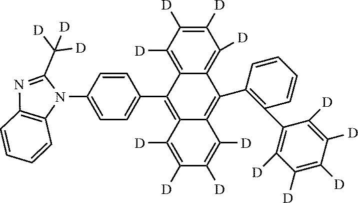 [SMILES: [2H]c1c([2H])c([2H])c(-c2ccccc2-c2c3c([2H])c([2H])c([2H])c([2H])c3c(-c3ccc(-n4c(C([2H])([2H])[2H])nc5ccccc54)cc3)c3c([2H])c([2H])c([2H])c([2H])c23)c([2H])c1[2H]]

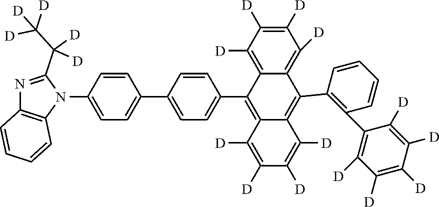 [SMILES: [2H]c1c([2H])c([2H])c(-c2ccccc2-c2c3c([2H])c([2H])c([2H])c([2H])c3c(-c3ccc(-c4ccc(-n5c(C([2H])([2H])C([2H])([2H])[2H])nc6ccccc65)cc4)cc3)c3c([2H])c([2H])c([2H])c([2H])c23)c([2H])c1[2H]]

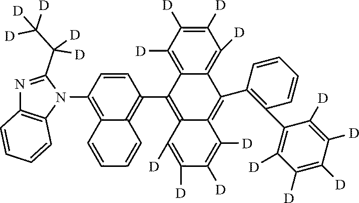 [SMILES: [2H]c1c([2H])c([2H])c(-c2ccccc2-c2c3c([2H])c([2H])c([2H])c([2H])c3c(-c3ccc(-n4c(C([2H])([2H])C([2H])([2H])[2H])nc5ccccc54)c4ccccc34)c3c([2H])c([2H])c([2H])c([2H])c23)c([2H])c1[2H]]

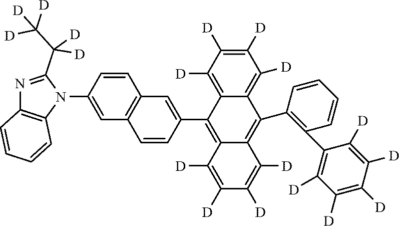 [SMILES: [2H]c1c([2H])c([2H])c(-c2ccccc2-c2c3c([2H])c([2H])c([2H])c([2H])c3c(-c3ccc4cc(-n5c(C([2H])([2H])C([2H])([2H])[2H])nc6ccccc65)ccc4c3)c3c([2H])c([2H])c([2H])c([2H])c23)c([2H])c1[2H]]

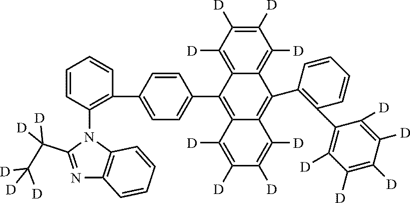 [SMILES: [2H]c1c([2H])c([2H])c(-c2ccccc2-c2c3c([2H])c([2H])c([2H])c([2H])c3c(-c3ccc(-c4ccccc4-n4c(C([2H])([2H])C([2H])([2H])[2H])nc5ccccc54)cc3)c3c([2H])c([2H])c([2H])c([2H])c23)c([2H])c1[2H]]

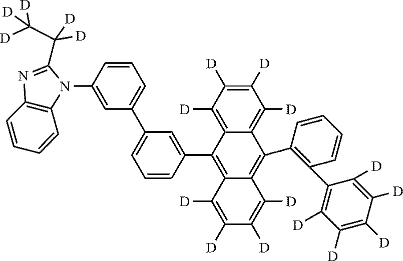 [SMILES: [2H]c1c([2H])c([2H])c(-c2ccccc2-c2c3c([2H])c([2H])c([2H])c([2H])c3c(-c3cccc(-c4cccc(-n5c(C([2H])([2H])C([2H])([2H])[2H])nc6ccccc65)c4)c3)c3c([2H])c([2H])c([2H])c([2H])c23)c([2H])c1[2H]]

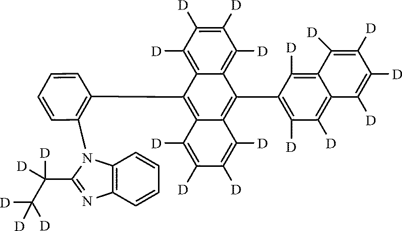 [SMILES: [2H]c1c([2H])c([2H])c2c([2H])c(-c3c4c([2H])c([2H])c([2H])c([2H])c4c(-c4ccccc4-n4c(C([2H])([2H])C([2H])([2H])[2H])nc5ccccc54)c4c([2H])c([2H])c([2H])c([2H])c34)c([2H])c([2H])c2c1[2H]]

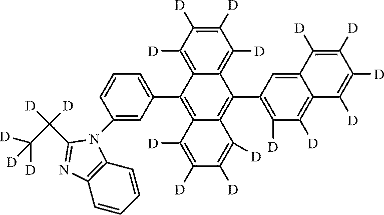 [SMILES: [2H]c1c([2H])c([2H])c2c([2H])c([2H])c(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)cc2c1[2H]]

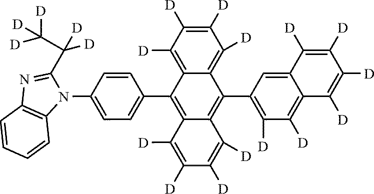 [SMILES: [2H]c1c([2H])c([2H])c2c([2H])c([2H])c(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)cc2c1[2H]]

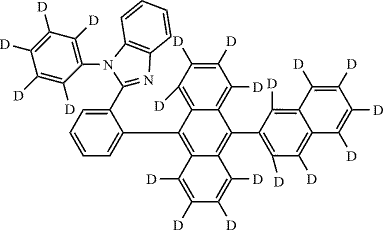 [SMILES: [2H]c1c([2H])c([2H])c(-n2c(-c3ccccc3-c3c4c([2H])c([2H])c([2H])c([2H])c4c(-c4c([2H])c([2H])c5c([2H])c([2H])c([2H])c([2H])c5c4[2H])c4c([2H])c([2H])c([2H])c([2H])c34)nc3ccccc32)c([2H])c1[2H]]

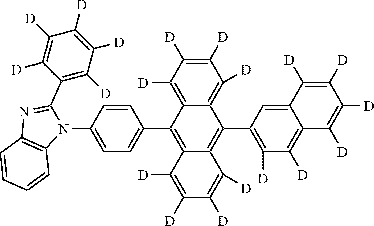 [SMILES: [2H]c1c([2H])c([2H])c(-c2nc3ccccc3n2-c2ccc(-c3c4c([2H])c([2H])c([2H])c([2H])c4c(-c4cc5c([2H])c([2H])c([2H])c([2H])c5c([2H])c4[2H])c4c([2H])c([2H])c([2H])c([2H])c34)cc2)c([2H])c1[2H]]

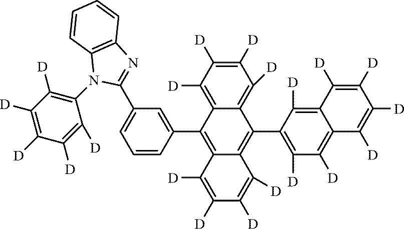 [SMILES: [2H]c1c([2H])c([2H])c(-n2c(-c3cccc(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c3)nc3ccccc32)c([2H])c1[2H]]

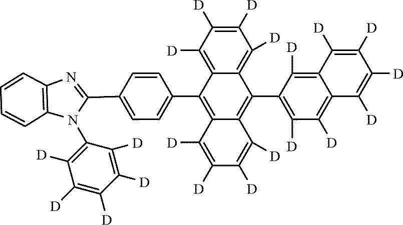 [SMILES: [2H]c1c([2H])c([2H])c(-n2c(-c3ccc(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)cc3)nc3ccccc32)c([2H])c1[2H]]

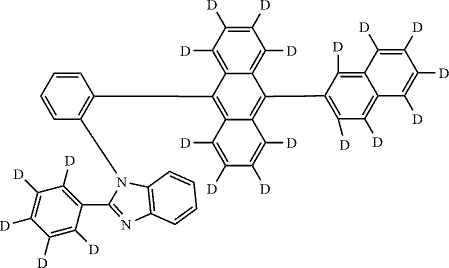 [SMILES: [2H]c1c([2H])c([2H])c(-c2nc3ccccc3n2-c2ccccc2-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c1[2H]]

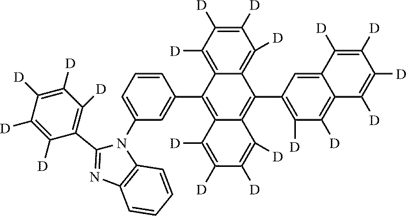 [SMILES: [2H]c1c([2H])c([2H])c(-c2nc3ccccc3n2-c2cccc(-c3c4c([2H])c([2H])c([2H])c([2H])c4c(-c4cc5c([2H])c([2H])c([2H])c([2H])c5c([2H])c4[2H])c4c([2H])c([2H])c([2H])c([2H])c34)c2)c([2H])c1[2H]]

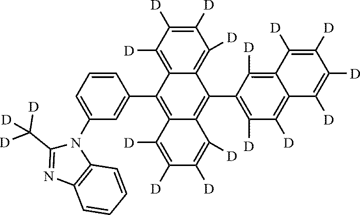 [SMILES: [2H]c1c([2H])c([2H])c2c([2H])c(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)c([2H])c([2H])c2c1[2H]]

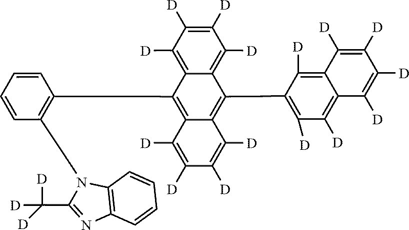 [SMILES: [2H]c1c([2H])c([2H])c2c([2H])c(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)c([2H])c([2H])c2c1[2H]]

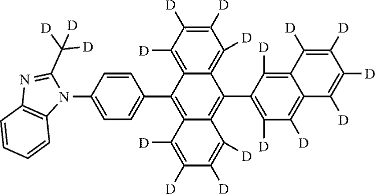 [SMILES: [2H]c1c([2H])c([2H])c2c([2H])c(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)c([2H])c([2H])c2c1[2H]]

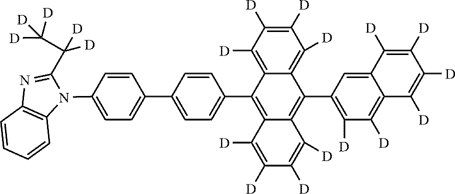 [SMILES: [2H]c1c([2H])c([2H])c2c([2H])c([2H])c(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)cc2c1[2H]]

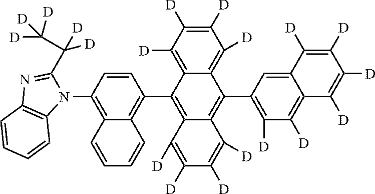 [SMILES: [2H]c1c([2H])c([2H])c2c([2H])c([2H])c(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)cc2c1[2H]]

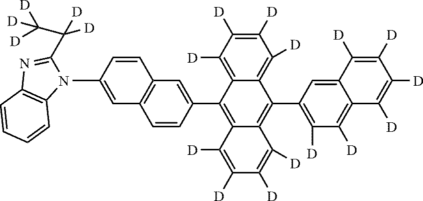 [SMILES: [2H]c1c([2H])c([2H])c2c([2H])c([2H])c(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)cc2c1[2H]]

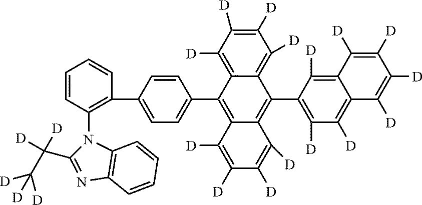 [SMILES: [2H]c1c([2H])c([2H])c2c([2H])c(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)c([2H])c([2H])c2c1[2H]]

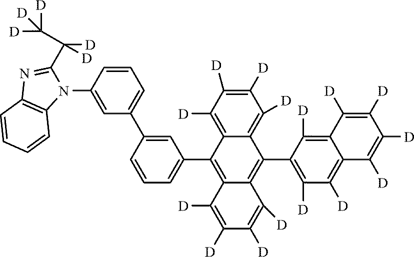 [SMILES: [2H]c1c([2H])c([2H])c2c([2H])c(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)c([2H])c([2H])c2c1[2H]]

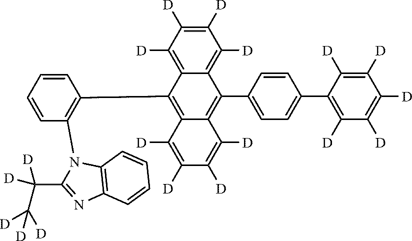 [SMILES: [2H]c1c([2H])c([2H])c(-c2ccc(-c3c4c([2H])c([2H])c([2H])c([2H])c4c(-c4ccccc4-n4c(C([2H])([2H])C([2H])([2H])[2H])nc5ccccc54)c4c([2H])c([2H])c([2H])c([2H])c34)cc2)c([2H])c1[2H]]

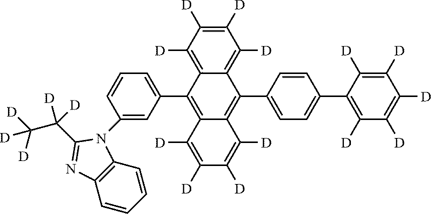 [SMILES: [2H]c1c([2H])c([2H])c(-c2ccc(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)cc2)c([2H])c1[2H]]

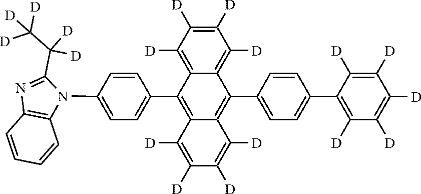 [SMILES: [2H]c1c([2H])c([2H])c(-c2ccc(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)cc2)c([2H])c1[2H]]

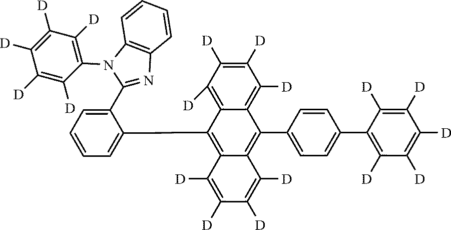 [SMILES: [2H]c1c([2H])c([2H])c(-c2ccc(-c3c4c([2H])c([2H])c([2H])c([2H])c4c(-c4ccccc4-c4nc5ccccc5n4-c4c([2H])c([2H])c([2H])c([2H])c4[2H])c4c([2H])c([2H])c([2H])c([2H])c34)cc2)c([2H])c1[2H]]

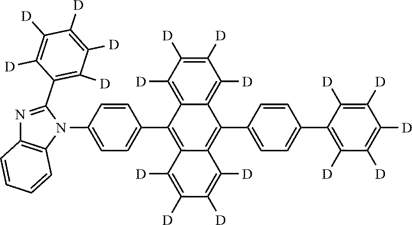 [SMILES: [2H]c1c([2H])c([2H])c(-c2ccc(-c3c4c([2H])c([2H])c([2H])c([2H])c4c(-c4ccc(-n5c(-c6c([2H])c([2H])c([2H])c([2H])c6[2H])nc6ccccc65)cc4)c4c([2H])c([2H])c([2H])c([2H])c34)cc2)c([2H])c1[2H]]

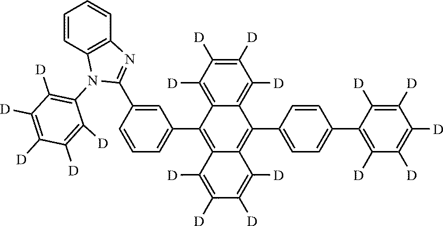 [SMILES: [2H]c1c([2H])c([2H])c(-c2ccc(-c3c4c([2H])c([2H])c([2H])c([2H])c4c(-c4cccc(-c5nc6ccccc6n5-c5c([2H])c([2H])c([2H])c([2H])c5[2H])c4)c4c([2H])c([2H])c([2H])c([2H])c34)cc2)c([2H])c1[2H]]

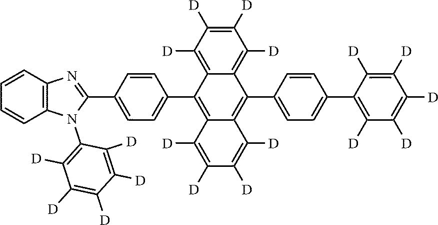 [SMILES: [2H]c1c([2H])c([2H])c(-c2ccc(-c3c4c([2H])c([2H])c([2H])c([2H])c4c(-c4ccc(-c5nc6ccccc6n5-c5c([2H])c([2H])c([2H])c([2H])c5[2H])cc4)c4c([2H])c([2H])c([2H])c([2H])c34)cc2)c([2H])c1[2H]]

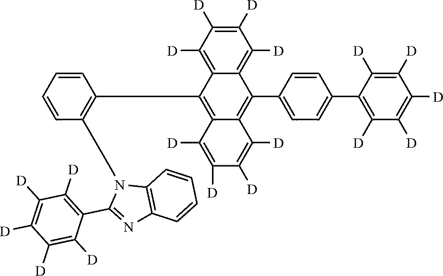 [SMILES: [2H]c1c([2H])c([2H])c(-c2ccc(-c3c4c([2H])c([2H])c([2H])c([2H])c4c(-c4ccccc4-n4c(-c5c([2H])c([2H])c([2H])c([2H])c5[2H])nc5ccccc54)c4c([2H])c([2H])c([2H])c([2H])c34)cc2)c([2H])c1[2H]]

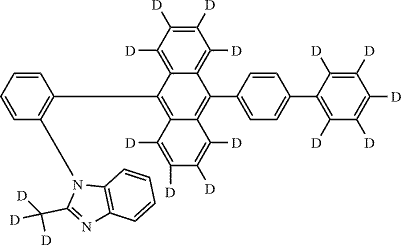 [SMILES: [2H]c1c([2H])c([2H])c(-c2ccc(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)cc2)c([2H])c1[2H]]

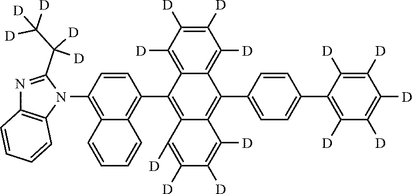 [SMILES: [2H]c1c([2H])c([2H])c(-c2ccc(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)cc2)c([2H])c1[2H]]

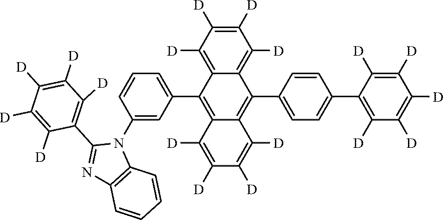 [SMILES: [2H]c1c([2H])c([2H])c(-c2ccc(-c3c4c([2H])c([2H])c([2H])c([2H])c4c(-c4cccc(-n5c(-c6c([2H])c([2H])c([2H])c([2H])c6[2H])nc6ccccc65)c4)c4c([2H])c([2H])c([2H])c([2H])c34)cc2)c([2H])c1[2H]]

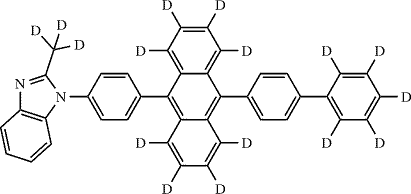 [SMILES: [2H]c1c([2H])c([2H])c(-c2ccc(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)cc2)c([2H])c1[2H]]

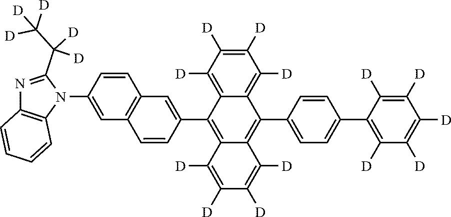 [SMILES: [2H]c1c([2H])c([2H])c(-c2ccc(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)cc2)c([2H])c1[2H]]

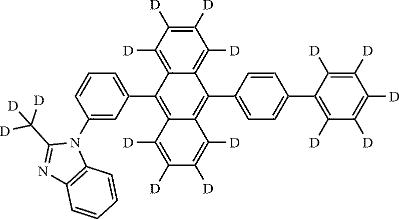 [SMILES: [2H]c1c([2H])c([2H])c(-c2ccc(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)cc2)c([2H])c1[2H]]

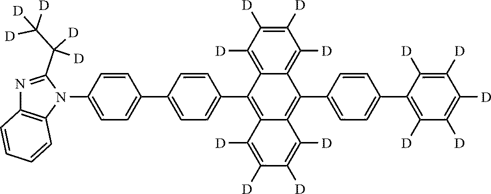 [SMILES: [2H]c1c([2H])c([2H])c(-c2ccc(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)cc2)c([2H])c1[2H]]

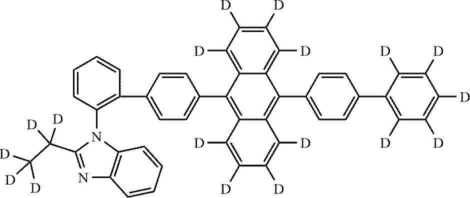 [SMILES: [2H]c1c([2H])c([2H])c(-c2ccc(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)cc2)c([2H])c1[2H]]

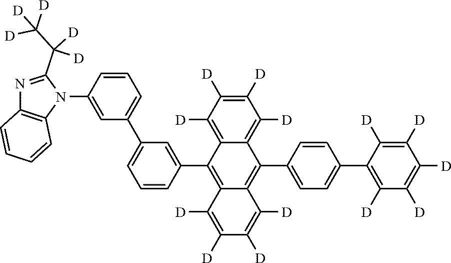 [SMILES: [2H]c1c([2H])c([2H])c(-c2ccc(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)cc2)c([2H])c1[2H]]

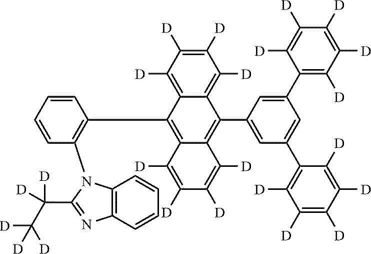 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(C([2H])([2H])C([2H])([2H])[2H])nc5ccccc54)c4c([2H])c([2H])c([2H])c([2H])c34)c2)c([2H])c1[2H]]

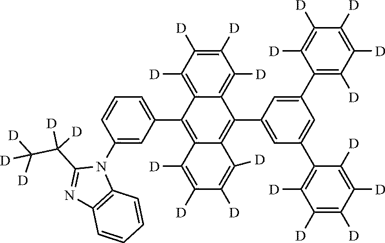 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)c2)c([2H])c1[2H]]

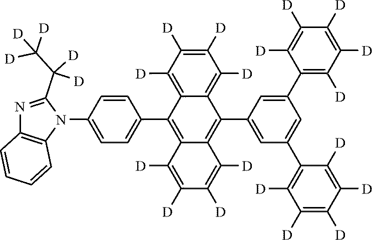 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)c2)c([2H])c1[2H]]

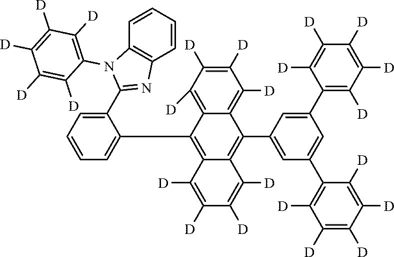 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-c4nc5ccccc5n4-c4c([2H])c([2H])c([2H])c([2H])c4[2H])c4c([2H])c([2H])c([2H])c([2H])c34)c2)c([2H])c1[2H]]

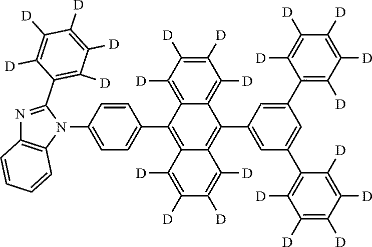 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(-c6c([2H])c([2H])c([2H])c([2H])c6[2H])nc6ccccc65)cc4)c4c([2H])c([2H])c([2H])c([2H])c34)c2)c([2H])c1[2H]]

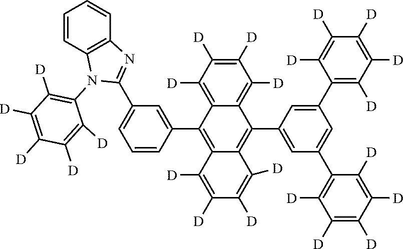 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-c5nc6ccccc6n5-c5c([2H])c([2H])c([2H])c([2H])c5[2H])c4)c4c([2H])c([2H])c([2H])c([2H])c34)c2)c([2H])c1[2H]]

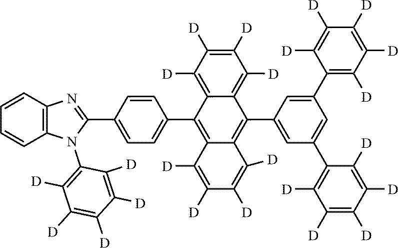 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5nc6ccccc6n5-c5c([2H])c([2H])c([2H])c([2H])c5[2H])cc4)c4c([2H])c([2H])c([2H])c([2H])c34)c2)c([2H])c1[2H]]

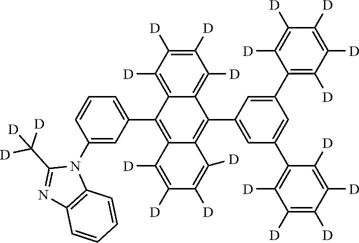 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)c2)c([2H])c1[2H]]

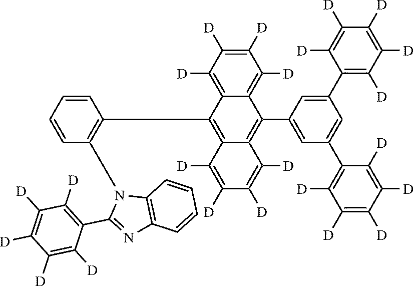 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(-c5c([2H])c([2H])c([2H])c([2H])c5[2H])nc5ccccc54)c4c([2H])c([2H])c([2H])c([2H])c34)c2)c([2H])c1[2H]]

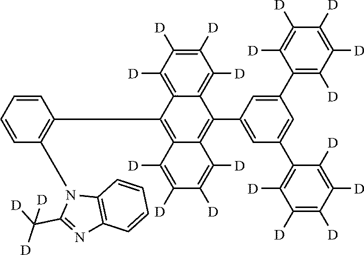 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)c2)c([2H])c1[2H]]

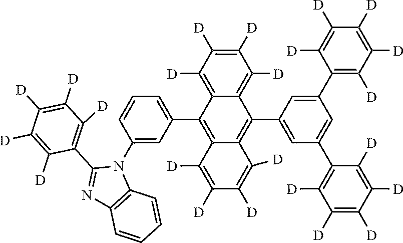 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(-c6c([2H])c([2H])c([2H])c([2H])c6[2H])nc6ccccc65)c4)c4c([2H])c([2H])c([2H])c([2H])c34)c2)c([2H])c1[2H]]

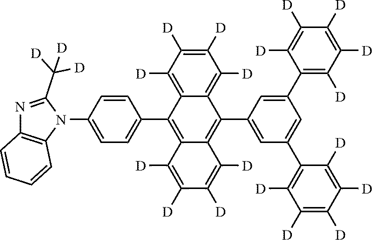 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)c2)c([2H])c1[2H]]

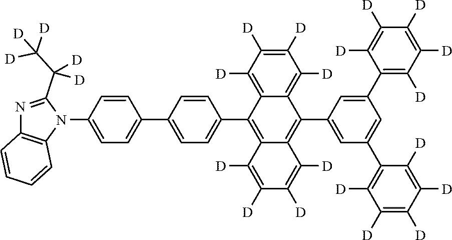 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)c2)c([2H])c1[2H]]

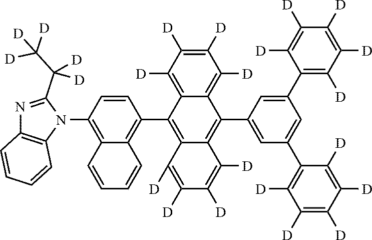 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)c2)c([2H])c1[2H]]

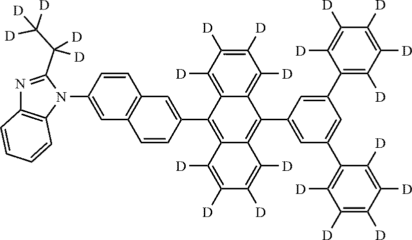 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)c2)c([2H])c1[2H]]

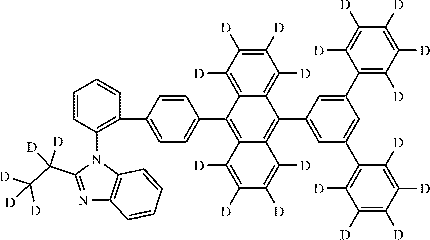 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)c2)c([2H])c1[2H]]

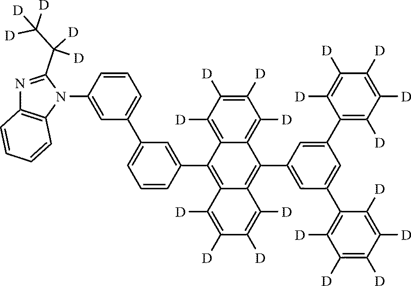 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)c2)c([2H])c1[2H]]

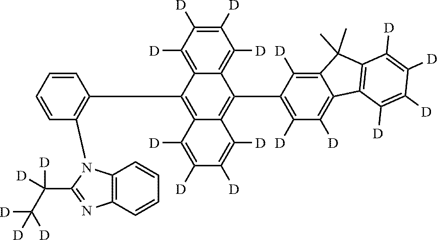 [SMILES: [2H]c1c([2H])c([2H])c2c(c1[2H])-c1c([2H])c([2H])c(-c3c4c([2H])c([2H])c([2H])c([2H])c4c(-c4ccccc4-n4c(C([2H])([2H])C([2H])([2H])[2H])nc5ccccc54)c4c([2H])c([2H])c([2H])c([2H])c34)c([2H])c1C2(C)C]

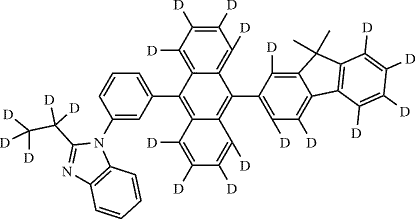 [SMILES: [2H]c1c([2H])c([2H])c2c(c1[2H])-c1c([2H])c([2H])c(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)c([2H])c1C2(C)C]

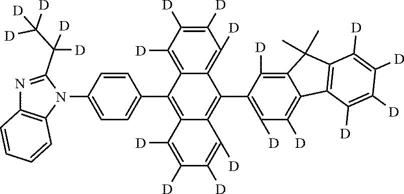 [SMILES: [2H]c1c([2H])c([2H])c2c(c1[2H])-c1c([2H])c([2H])c(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)c([2H])c1C2(C)C]

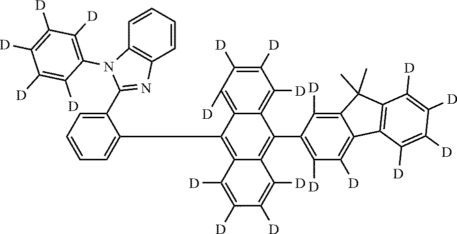 [SMILES: [2H]c1c([2H])c([2H])c(-n2c(-c3ccccc3-c3c4c([2H])c([2H])c([2H])c([2H])c4c(-c4c([2H])c([2H])c5c(c4[2H])C(C)(C)c4c([2H])c([2H])c([2H])c([2H])c4-5)c4c([2H])c([2H])c([2H])c([2H])c34)nc3ccccc32)c([2H])c1[2H]]

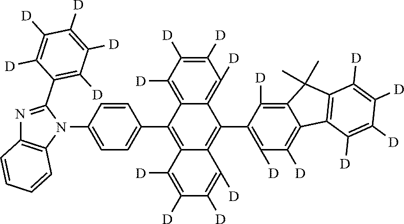 [SMILES: [2H]c1c([2H])c([2H])c(-c2nc3ccccc3n2-c2ccc(-c3c4c([2H])c([2H])c([2H])c([2H])c4c(-c4c([2H])c([2H])c5c(c4[2H])C(C)(C)c4c([2H])c([2H])c([2H])c([2H])c4-5)c4c([2H])c([2H])c([2H])c([2H])c34)cc2)c([2H])c1[2H]]

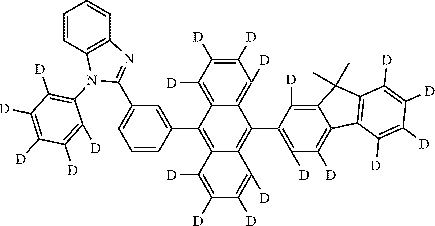 [SMILES: [2H]c1c([2H])c([2H])c(-n2c(-c3cccc(-c4c5c([2H])c([2H])c([2H])c([2H])c5c(-c5c([2H])c([2H])c6c(c5[2H])C(C)(C)c5c([2H])c([2H])c([2H])c([2H])c5-6)c5c([2H])c([2H])c([2H])c([2H])c45)c3)nc3ccccc32)c([2H])c1[2H]]

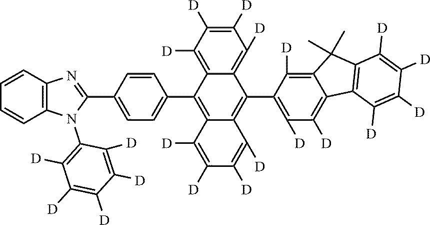 [SMILES: [2H]c1c([2H])c([2H])c(-n2c(-c3ccc(-c4c5c([2H])c([2H])c([2H])c([2H])c5c(-c5c([2H])c([2H])c6c(c5[2H])C(C)(C)c5c([2H])c([2H])c([2H])c([2H])c5-6)c5c([2H])c([2H])c([2H])c([2H])c45)cc3)nc3ccccc32)c([2H])c1[2H]]

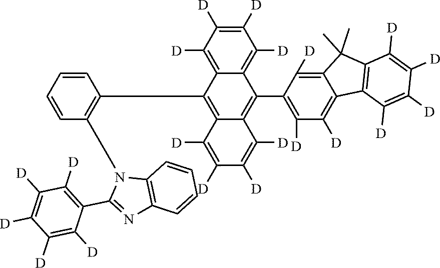 [SMILES: [2H]c1c([2H])c([2H])c(-c2nc3ccccc3n2-c2ccccc2-c2c3c([2H])c([2H])c([2H])c([2H])c3c(-c3c([2H])c([2H])c4c(c3[2H])C(C)(C)c3c([2H])c([2H])c([2H])c([2H])c3-4)c3c([2H])c([2H])c([2H])c([2H])c23)c([2H])c1[2H]]

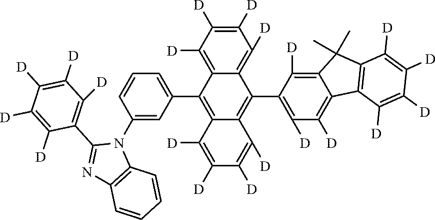 [SMILES: [2H]c1c([2H])c([2H])c(-c2nc3ccccc3n2-c2cccc(-c3c4c([2H])c([2H])c([2H])c([2H])c4c(-c4c([2H])c([2H])c5c(c4[2H])C(C)(C)c4c([2H])c([2H])c([2H])c([2H])c4-5)c4c([2H])c([2H])c([2H])c([2H])c34)c2)c([2H])c1[2H]]

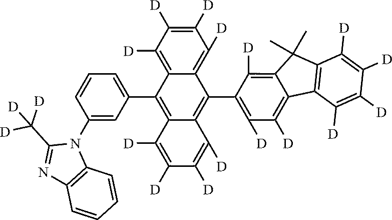 [SMILES: [2H]c1c([2H])c([2H])c2c(c1[2H])-c1c([2H])c([2H])c(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)c([2H])c1C2(C)C]

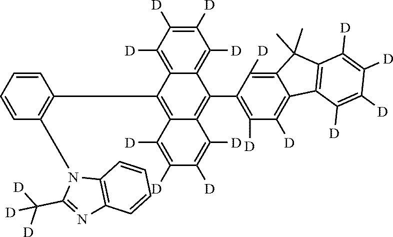 [SMILES: [2H]c1c([2H])c([2H])c2c(c1[2H])-c1c([2H])c([2H])c(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)c([2H])c1C2(C)C]

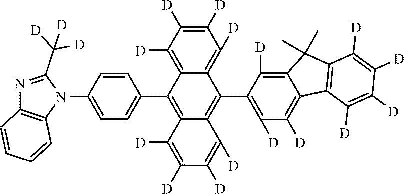 [SMILES: [2H]c1c([2H])c([2H])c2c(c1[2H])-c1c([2H])c([2H])c(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)c([2H])c1C2(C)C]

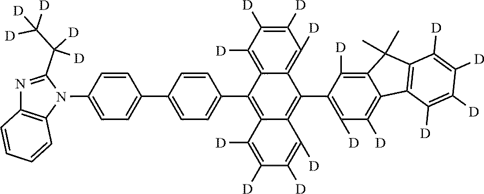 [SMILES: [2H]c1c([2H])c([2H])c2c(c1[2H])-c1c([2H])c([2H])c(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)c([2H])c1C2(C)C]

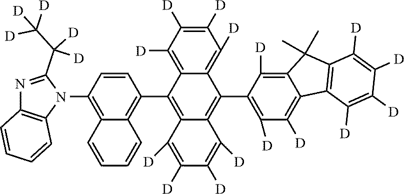 [SMILES: [2H]c1c([2H])c([2H])c2c(c1[2H])-c1c([2H])c([2H])c(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)c([2H])c1C2(C)C]

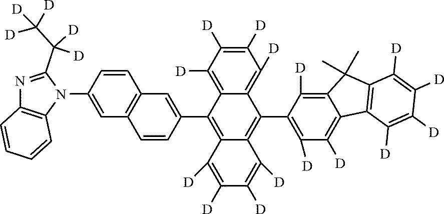 [SMILES: [2H]c1c([2H])c([2H])c2c(c1[2H])-c1c([2H])c([2H])c(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)c([2H])c1C2(C)C]

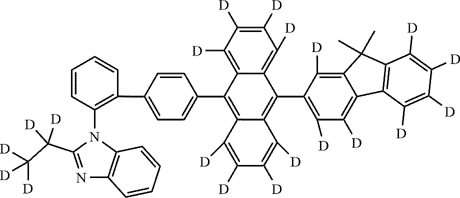 [SMILES: [2H]c1c([2H])c([2H])c2c(c1[2H])-c1c([2H])c([2H])c(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)c([2H])c1C2(C)C]

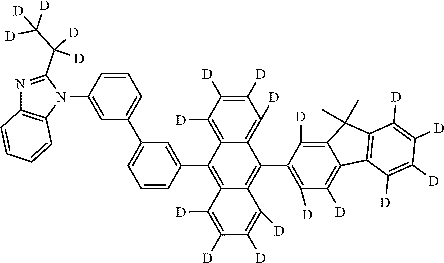 [SMILES: [2H]c1c([2H])c([2H])c2c(c1[2H])-c1c([2H])c([2H])c(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)c([2H])c1C2(C)C]

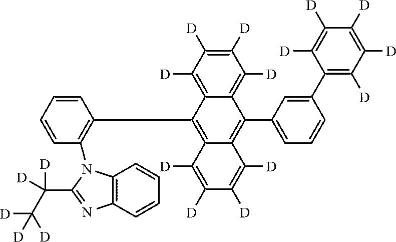 [SMILES: [2H]c1c([2H])c([2H])c(-c2cccc(-c3c4c([2H])c([2H])c([2H])c([2H])c4c(-c4ccccc4-n4c(C([2H])([2H])C([2H])([2H])[2H])nc5ccccc54)c4c([2H])c([2H])c([2H])c([2H])c34)c2)c([2H])c1[2H]]

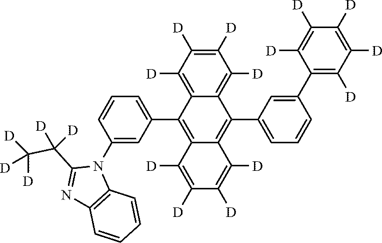 [SMILES: [2H]c1c([2H])c([2H])c(-c2cccc(-c3c4c([2H])c([2H])c([2H])c([2H])c4c(-c4cccc(-n5c(C([2H])([2H])C([2H])([2H])[2H])nc6ccccc65)c4)c4c([2H])c([2H])c([2H])c([2H])c34)c2)c([2H])c1[2H]]

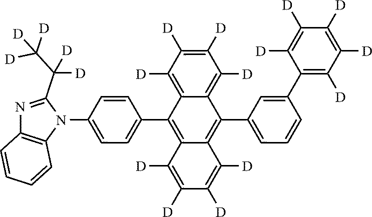 [SMILES: [2H]c1c([2H])c([2H])c(-c2cccc(-c3c4c([2H])c([2H])c([2H])c([2H])c4c(-c4ccc(-n5c(C([2H])([2H])C([2H])([2H])[2H])nc6ccccc65)cc4)c4c([2H])c([2H])c([2H])c([2H])c34)c2)c([2H])c1[2H]]

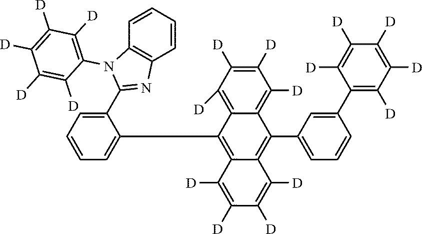 [SMILES: [2H]c1c([2H])c([2H])c(-c2cccc(-c3c4c([2H])c([2H])c([2H])c([2H])c4c(-c4ccccc4-c4nc5ccccc5n4-c4c([2H])c([2H])c([2H])c([2H])c4[2H])c4c([2H])c([2H])c([2H])c([2H])c34)c2)c([2H])c1[2H]]

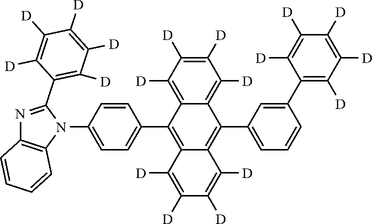 [SMILES: [2H]c1c([2H])c([2H])c(-c2cccc(-c3c4c([2H])c([2H])c([2H])c([2H])c4c(-c4ccc(-n5c(-c6c([2H])c([2H])c([2H])c([2H])c6[2H])nc6ccccc65)cc4)c4c([2H])c([2H])c([2H])c([2H])c34)c2)c([2H])c1[2H]]

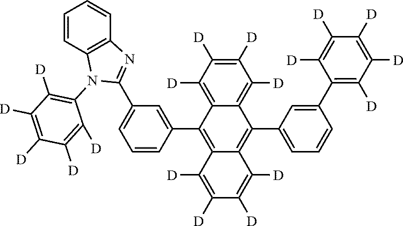 [SMILES: [2H]c1c([2H])c([2H])c(-c2cccc(-c3c4c([2H])c([2H])c([2H])c([2H])c4c(-c4cccc(-c5nc6ccccc6n5-c5c([2H])c([2H])c([2H])c([2H])c5[2H])c4)c4c([2H])c([2H])c([2H])c([2H])c34)c2)c([2H])c1[2H]]

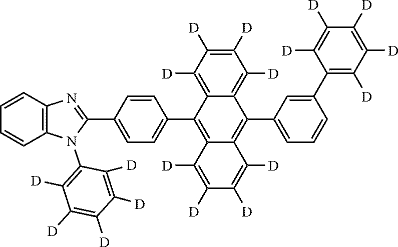 [SMILES: [2H]c1c([2H])c([2H])c(-c2cccc(-c3c4c([2H])c([2H])c([2H])c([2H])c4c(-c4ccc(-c5nc6ccccc6n5-c5c([2H])c([2H])c([2H])c([2H])c5[2H])cc4)c4c([2H])c([2H])c([2H])c([2H])c34)c2)c([2H])c1[2H]]

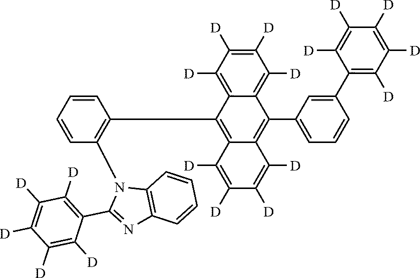 [SMILES: [2H]c1c([2H])c([2H])c(-c2cccc(-c3c4c([2H])c([2H])c([2H])c([2H])c4c(-c4ccccc4-n4c(-c5c([2H])c([2H])c([2H])c([2H])c5[2H])nc5ccccc54)c4c([2H])c([2H])c([2H])c([2H])c34)c2)c([2H])c1[2H]]

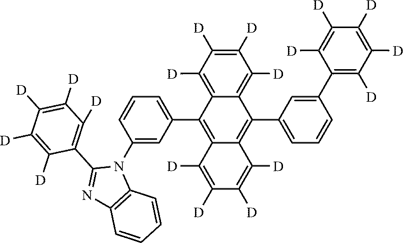 [SMILES: [2H]c1c([2H])c([2H])c(-c2cccc(-c3c4c([2H])c([2H])c([2H])c([2H])c4c(-c4cccc(-n5c(-c6c([2H])c([2H])c([2H])c([2H])c6[2H])nc6ccccc65)c4)c4c([2H])c([2H])c([2H])c([2H])c34)c2)c([2H])c1[2H]]

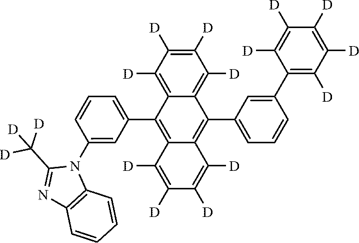 [SMILES: [2H]c1c([2H])c([2H])c(-c2cccc(-c3c4c([2H])c([2H])c([2H])c([2H])c4c(-c4cccc(-n5c(C([2H])([2H])[2H])nc6ccccc65)c4)c4c([2H])c([2H])c([2H])c([2H])c34)c2)c([2H])c1[2H]]

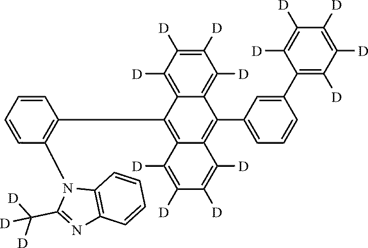 [SMILES: [2H]c1c([2H])c([2H])c(-c2cccc(-c3c4c([2H])c([2H])c([2H])c([2H])c4c(-c4ccccc4-n4c(C([2H])([2H])[2H])nc5ccccc54)c4c([2H])c([2H])c([2H])c([2H])c34)c2)c([2H])c1[2H]]

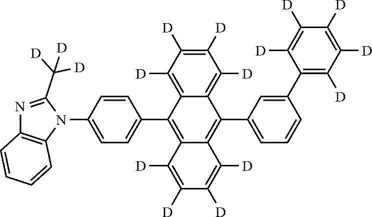 [SMILES: [2H]c1c([2H])c([2H])c(-c2cccc(-c3c4c([2H])c([2H])c([2H])c([2H])c4c(-c4ccc(-n5c(C([2H])([2H])[2H])nc6ccccc65)cc4)c4c([2H])c([2H])c([2H])c([2H])c34)c2)c([2H])c1[2H]]

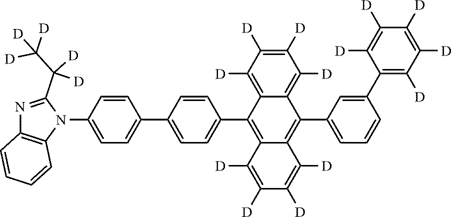 [SMILES: [2H]c1c([2H])c([2H])c(-c2cccc(-c3c4c([2H])c([2H])c([2H])c([2H])c4c(-c4ccc(-c5ccc(-n6c(C([2H])([2H])C([2H])([2H])[2H])nc7ccccc76)cc5)cc4)c4c([2H])c([2H])c([2H])c([2H])c34)c2)c([2H])c1[2H]]

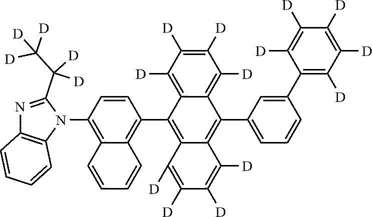 [SMILES: [2H]c1c([2H])c([2H])c(-c2cccc(-c3c4c([2H])c([2H])c([2H])c([2H])c4c(-c4ccc(-n5c(C([2H])([2H])C([2H])([2H])[2H])nc6ccccc65)c5ccccc45)c4c([2H])c([2H])c([2H])c([2H])c34)c2)c([2H])c1[2H]]

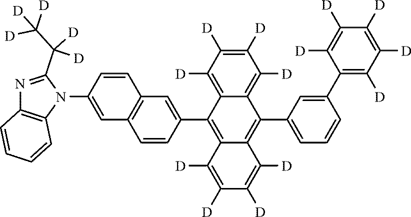 [SMILES: [2H]c1c([2H])c([2H])c(-c2cccc(-c3c4c([2H])c([2H])c([2H])c([2H])c4c(-c4ccc5cc(-n6c(C([2H])([2H])C([2H])([2H])[2H])nc7ccccc76)ccc5c4)c4c([2H])c([2H])c([2H])c([2H])c34)c2)c([2H])c1[2H]]

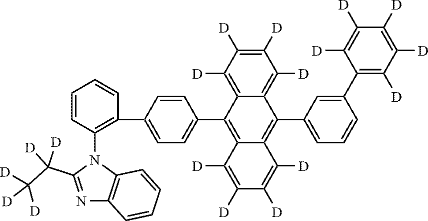 [SMILES: [2H]c1c([2H])c([2H])c(-c2cccc(-c3c4c([2H])c([2H])c([2H])c([2H])c4c(-c4ccc(-c5ccccc5-n5c(C([2H])([2H])C([2H])([2H])[2H])nc6ccccc65)cc4)c4c([2H])c([2H])c([2H])c([2H])c34)c2)c([2H])c1[2H]]

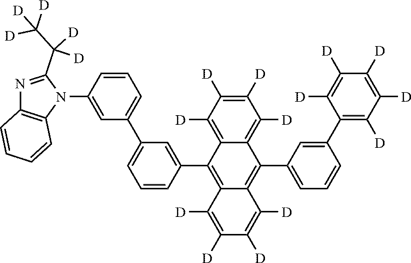 [SMILES: [2H]c1c([2H])c([2H])c(-c2cccc(-c3c4c([2H])c([2H])c([2H])c([2H])c4c(-c4cccc(-c5cccc(-n6c(C([2H])([2H])C([2H])([2H])[2H])nc7ccccc76)c5)c4)c4c([2H])c([2H])c([2H])c([2H])c34)c2)c([2H])c1[2H]]

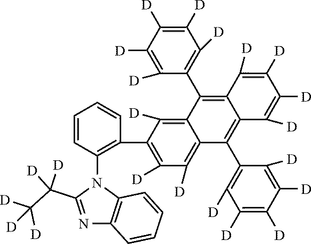 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(C([2H])([2H])C([2H])([2H])[2H])nc5ccccc54)c([2H])c([2H])c23)c([2H])c1[2H]]

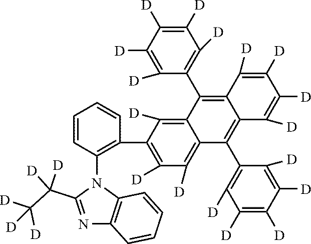 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(C([2H])([2H])C([2H])([2H])[2H])nc5ccccc54)c([2H])c([2H])c23)c([2H])c1[2H]]

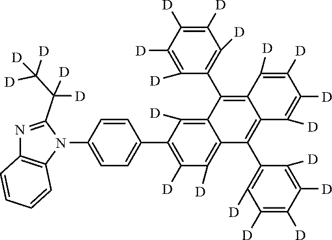 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(C([2H])([2H])C([2H])([2H])[2H])nc6ccccc65)cc4)c([2H])c([2H])c23)c([2H])c1[2H]]

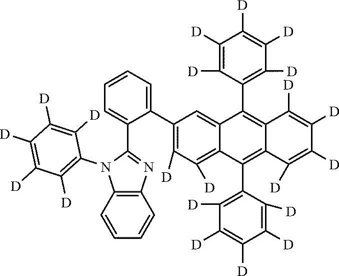 [SMILES: [2H]c1c([2H])c([2H])c(-c2c3cc(-c4ccccc4-c4nc5ccccc5n4-c4c([2H])c([2H])c([2H])c([2H])c4[2H])c([2H])c([2H])c3c(-c3c([2H])c([2H])c([2H])c([2H])c3[2H])c3c([2H])c([2H])c([2H])c([2H])c23)c([2H])c1[2H]]

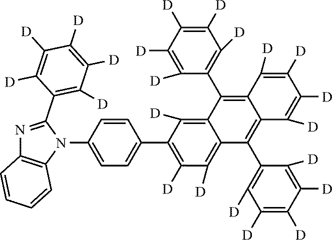 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(-c6c([2H])c([2H])c([2H])c([2H])c6[2H])nc6ccccc65)cc4)c([2H])c([2H])c23)c([2H])c1[2H]]

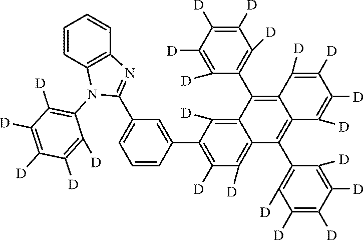 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-c5nc6ccccc6n5-c5c([2H])c([2H])c([2H])c([2H])c5[2H])c4)c([2H])c([2H])c23)c([2H])c1[2H]]

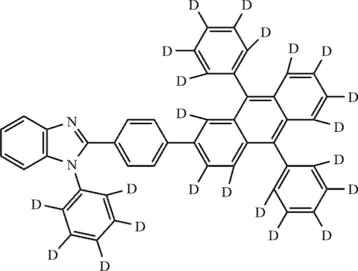 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5nc6ccccc6n5-c5c([2H])c([2H])c([2H])c([2H])c5[2H])cc4)c([2H])c([2H])c23)c([2H])c1[2H]]

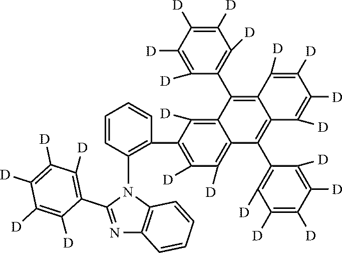 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(-c5c([2H])c([2H])c([2H])c([2H])c5[2H])nc5ccccc54)c([2H])c([2H])c23)c([2H])c1[2H]]

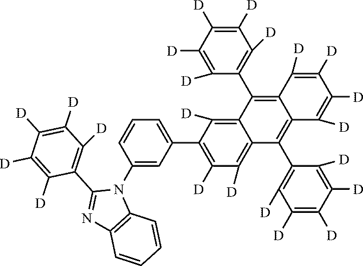 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-n5c(-c6c([2H])c([2H])c([2H])c([2H])c6[2H])nc6ccccc65)c4)c([2H])c([2H])c23)c([2H])c1[2H]]

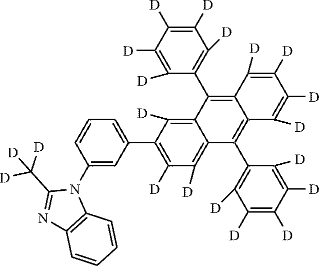 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-n5c(C([2H])([2H])[2H])nc6ccccc65)c4)c([2H])c([2H])c23)c([2H])c1[2H]]

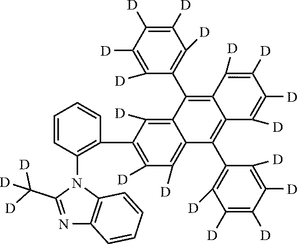 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(C([2H])([2H])[2H])nc5ccccc54)c([2H])c([2H])c23)c([2H])c1[2H]]

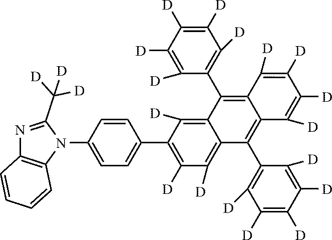 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(C([2H])([2H])[2H])nc6ccccc65)cc4)c([2H])c([2H])c23)c([2H])c1[2H]]

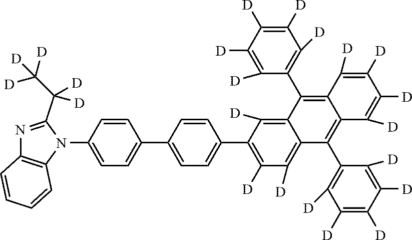 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5ccc(-n6c(C([2H])([2H])C([2H])([2H])[2H])nc7ccccc76)cc5)cc4)c([2H])c([2H])c23)c([2H])c1[2H]]

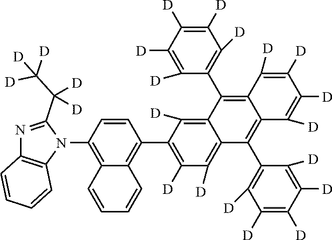 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(C([2H])([2H])C([2H])([2H])[2H])nc6ccccc65)c5ccccc45)c([2H])c([2H])c23)c([2H])c1[2H]]

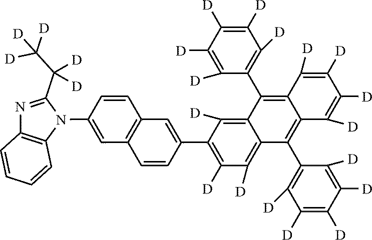

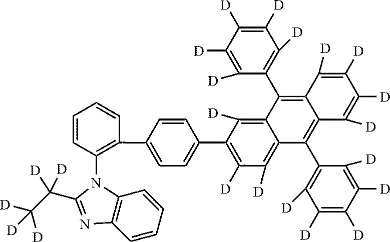 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5ccccc5-n5c(C([2H])([2H])C([2H])([2H])[2H])nc6ccccc65)cc4)c([2H])c([2H])c23)c([2H])c1[2H]]

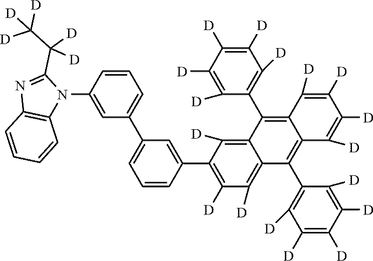 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-c5cccc(-n6c(C([2H])([2H])C([2H])([2H])[2H])nc7ccccc76)c5)c4)c([2H])c([2H])c23)c([2H])c1[2H]]

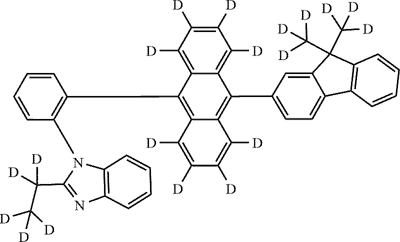 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

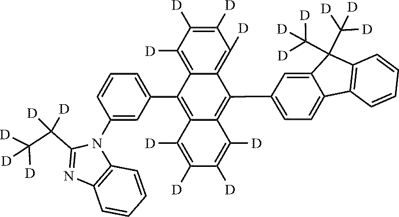 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-n4c(C([2H])([2H])C([2H])([2H])[2H])nc5ccccc54)c3)c2c1[2H]]

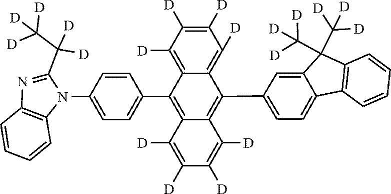 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-n4c(C([2H])([2H])C([2H])([2H])[2H])nc5ccccc54)cc3)c2c1[2H]]

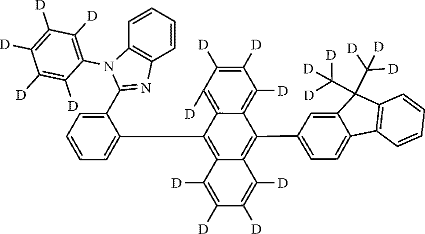 [SMILES: [2H]c1c([2H])c([2H])c(-n2c(-c3ccccc3-c3c4c([2H])c([2H])c([2H])c([2H])c4c(-c4ccc5c(c4)C(C([2H])([2H])[2H])(C([2H])([2H])[2H])c4ccccc4-5)c4c([2H])c([2H])c([2H])c([2H])c34)nc3ccccc32)c([2H])c1[2H]]

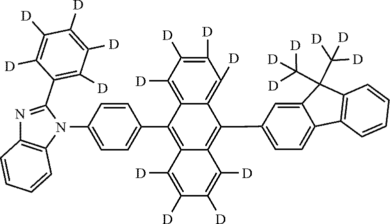 [SMILES: [2H]c1c([2H])c([2H])c(-c2nc3ccccc3n2-c2ccc(-c3c4c([2H])c([2H])c([2H])c([2H])c4c(-c4ccc5c(c4)C(C([2H])([2H])[2H])(C([2H])([2H])[2H])c4ccccc4-5)c4c([2H])c([2H])c([2H])c([2H])c34)cc2)c([2H])c1[2H]]

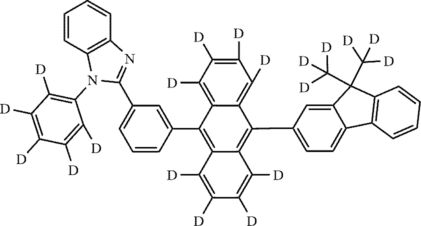 [SMILES: [2H]c1c([2H])c([2H])c(-n2c(-c3cccc(-c4c5c([2H])c([2H])c([2H])c([2H])c5c(-c5ccc6c(c5)C(C([2H])([2H])[2H])(C([2H])([2H])[2H])c5ccccc5-6)c5c([2H])c([2H])c([2H])c([2H])c45)c3)nc3ccccc32)c([2H])c1[2H]]

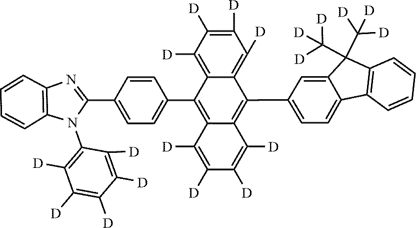 [SMILES: [2H]c1c([2H])c([2H])c(-n2c(-c3ccc(-c4c5c([2H])c([2H])c([2H])c([2H])c5c(-c5ccc6c(c5)C(C([2H])([2H])[2H])(C([2H])([2H])[2H])c5ccccc5-6)c5c([2H])c([2H])c([2H])c([2H])c45)cc3)nc3ccccc32)c([2H])c1[2H]]

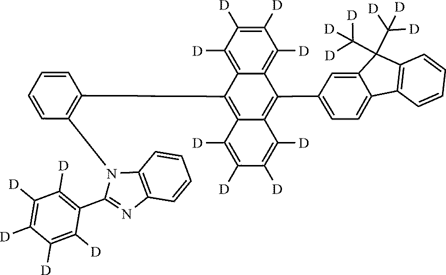 [SMILES: [2H]c1c([2H])c([2H])c(-c2nc3ccccc3n2-c2ccccc2-c2c3c([2H])c([2H])c([2H])c([2H])c3c(-c3ccc4c(c3)C(C([2H])([2H])[2H])(C([2H])([2H])[2H])c3ccccc3-4)c3c([2H])c([2H])c([2H])c([2H])c23)c([2H])c1[2H]]

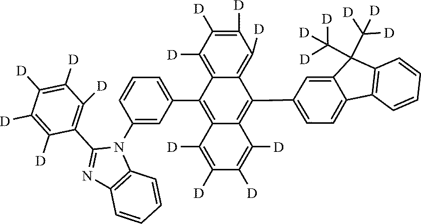 [SMILES: [2H]c1c([2H])c([2H])c(-c2nc3ccccc3n2-c2cccc(-c3c4c([2H])c([2H])c([2H])c([2H])c4c(-c4ccc5c(c4)C(C([2H])([2H])[2H])(C([2H])([2H])[2H])c4ccccc4-5)c4c([2H])c([2H])c([2H])c([2H])c34)c2)c([2H])c1[2H]]

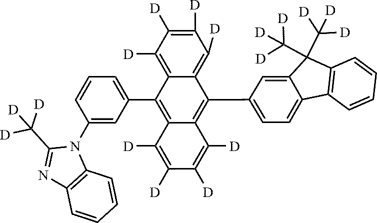 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-n4c(C([2H])([2H])[2H])nc5ccccc54)c3)c2c1[2H]]

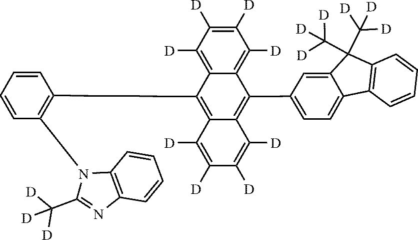 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

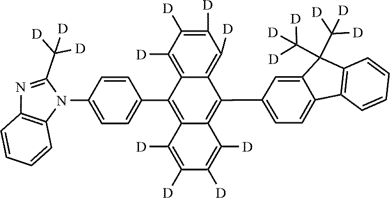 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-n4c(C([2H])([2H])[2H])nc5ccccc54)cc3)c2c1[2H]]

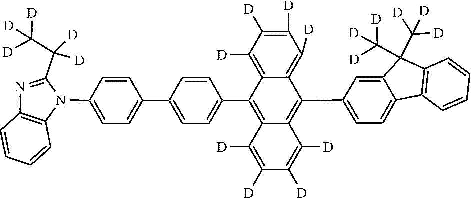 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

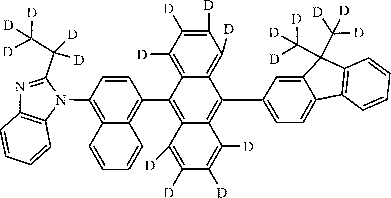 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccc4c(c3)C(C([2H])([2H])[2H])(C([2H])([2H])[2H])c3ccccc3-4)c2c1[2H]]

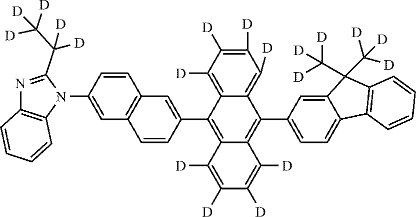 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3ccc4c(c3)C(C([2H])([2H])[2H])(C([2H])([2H])[2H])c3ccccc3-4)c2c1[2H]]

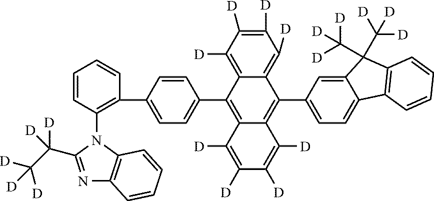 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

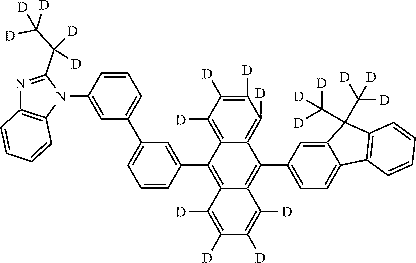 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

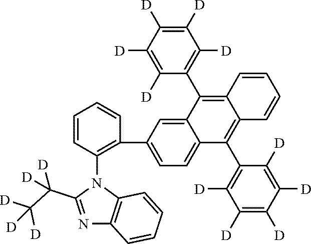 [SMILES: [2H]c1c([2H])c([2H])c(-c2c3ccccc3c(-c3c([2H])c([2H])c([2H])c([2H])c3[2H])c3cc(-c4ccccc4-n4c(C([2H])([2H])C([2H])([2H])[2H])nc5ccccc54)ccc23)c([2H])c1[2H]]

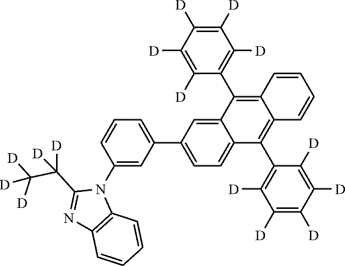 [SMILES: [2H]c1c([2H])c([2H])c(-c2c3ccccc3c(-c3c([2H])c([2H])c([2H])c([2H])c3[2H])c3cc(-c4cccc(-n5c(C([2H])([2H])C([2H])([2H])[2H])nc6ccccc65)c4)ccc23)c([2H])c1[2H]]

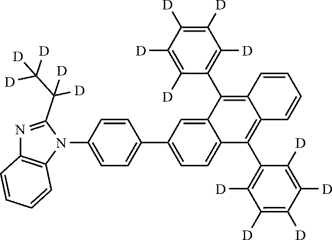 [SMILES: [2H]c1c([2H])c([2H])c(-c2c3ccccc3c(-c3c([2H])c([2H])c([2H])c([2H])c3[2H])c3cc(-c4ccc(-n5c(C([2H])([2H])C([2H])([2H])[2H])nc6ccccc65)cc4)ccc23)c([2H])c1[2H]]

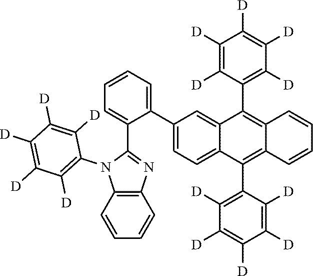 [SMILES: [2H]c1c([2H])c([2H])c(-c2c3ccccc3c(-c3c([2H])c([2H])c([2H])c([2H])c3[2H])c3cc(-c4ccccc4-c4nc5ccccc5n4-c4c([2H])c([2H])c([2H])c([2H])c4[2H])ccc23)c([2H])c1[2H]]

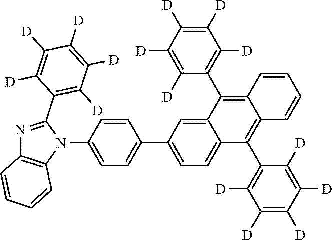 [SMILES: [2H]c1c([2H])c([2H])c(-c2c3ccccc3c(-c3c([2H])c([2H])c([2H])c([2H])c3[2H])c3cc(-c4ccc(-n5c(-c6c([2H])c([2H])c([2H])c([2H])c6[2H])nc6ccccc65)cc4)ccc23)c([2H])c1[2H]]

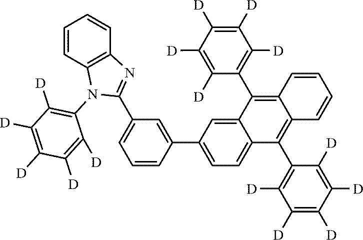 [SMILES: [2H]c1c([2H])c([2H])c(-c2c3ccccc3c(-c3c([2H])c([2H])c([2H])c([2H])c3[2H])c3cc(-c4cccc(-c5nc6ccccc6n5-c5c([2H])c([2H])c([2H])c([2H])c5[2H])c4)ccc23)c([2H])c1[2H]]

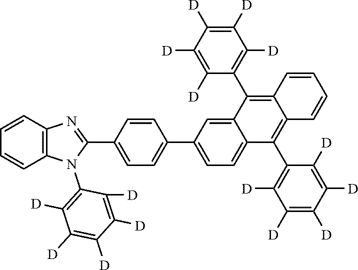 [SMILES: [2H]c1c([2H])c([2H])c(-c2c3ccccc3c(-c3c([2H])c([2H])c([2H])c([2H])c3[2H])c3cc(-c4ccc(-c5nc6ccccc6n5-c5c([2H])c([2H])c([2H])c([2H])c5[2H])cc4)ccc23)c([2H])c1[2H]]

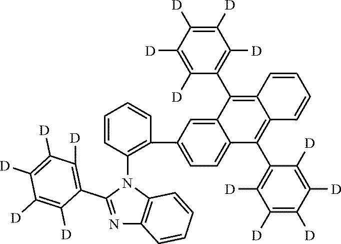 [SMILES: [2H]c1c([2H])c([2H])c(-c2c3ccccc3c(-c3c([2H])c([2H])c([2H])c([2H])c3[2H])c3cc(-c4ccccc4-n4c(-c5c([2H])c([2H])c([2H])c([2H])c5[2H])nc5ccccc54)ccc23)c([2H])c1[2H]]

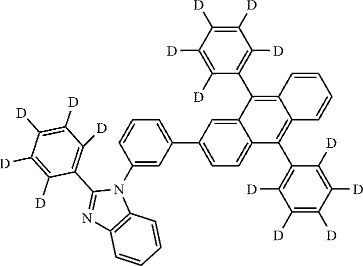 [SMILES: [2H]c1c([2H])c([2H])c(-c2c3ccccc3c(-c3c([2H])c([2H])c([2H])c([2H])c3[2H])c3cc(-c4cccc(-n5c(-c6c([2H])c([2H])c([2H])c([2H])c6[2H])nc6ccccc65)c4)ccc23)c([2H])c1[2H]]

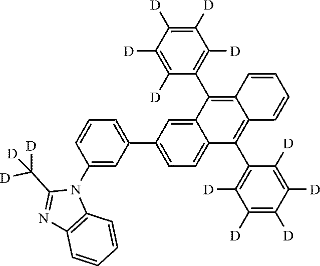 [SMILES: [2H]c1c([2H])c([2H])c(-c2c3ccccc3c(-c3c([2H])c([2H])c([2H])c([2H])c3[2H])c3cc(-c4cccc(-n5c(C([2H])([2H])[2H])nc6ccccc65)c4)ccc23)c([2H])c1[2H]]

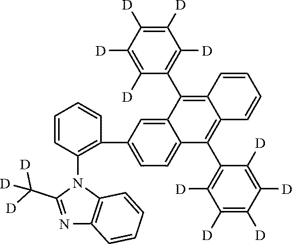 [SMILES: [2H]c1c([2H])c([2H])c(-c2c3ccccc3c(-c3c([2H])c([2H])c([2H])c([2H])c3[2H])c3cc(-c4ccccc4-n4c(C([2H])([2H])[2H])nc5ccccc54)ccc23)c([2H])c1[2H]]

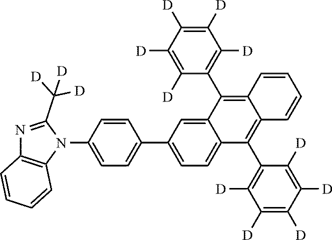 [SMILES: [2H]c1c([2H])c([2H])c(-c2c3ccccc3c(-c3c([2H])c([2H])c([2H])c([2H])c3[2H])c3cc(-c4ccc(-n5c(C([2H])([2H])[2H])nc6ccccc65)cc4)ccc23)c([2H])c1[2H]]

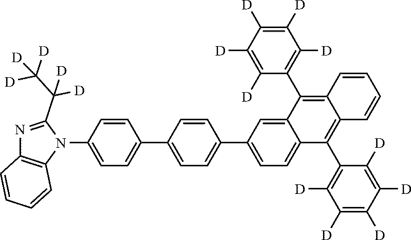 [SMILES: [2H]c1c([2H])c([2H])c(-c2c3ccccc3c(-c3c([2H])c([2H])c([2H])c([2H])c3[2H])c3cc(-c4ccc(-c5ccc(-n6c(C([2H])([2H])C([2H])([2H])[2H])nc7ccccc76)cc5)cc4)ccc23)c([2H])c1[2H]]

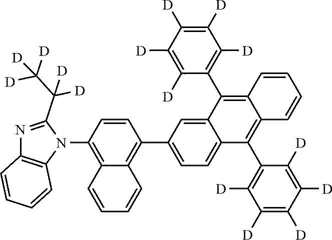 [SMILES: [2H]c1c([2H])c([2H])c(-c2c3ccccc3c(-c3c([2H])c([2H])c([2H])c([2H])c3[2H])c3cc(-c4ccc(-n5c(C([2H])([2H])C([2H])([2H])[2H])nc6ccccc65)c5ccccc45)ccc23)c([2H])c1[2H]]

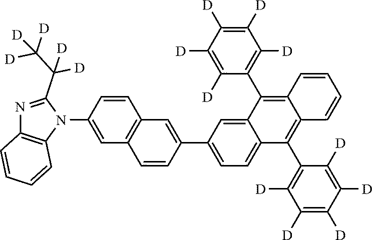 [SMILES: [2H]c1c([2H])c([2H])c(-c2c3ccccc3c(-c3c([2H])c([2H])c([2H])c([2H])c3[2H])c3cc(-c4ccc5cc(-n6c(C([2H])([2H])C([2H])([2H])[2H])nc7ccccc76)ccc5c4)ccc23)c([2H])c1[2H]]

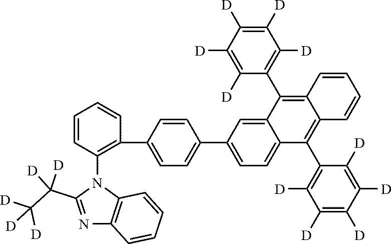 [SMILES: [2H]c1c([2H])c([2H])c(-c2c3ccccc3c(-c3c([2H])c([2H])c([2H])c([2H])c3[2H])c3cc(-c4ccc(-c5ccccc5-n5c(C([2H])([2H])C([2H])([2H])[2H])nc6ccccc65)cc4)ccc23)c([2H])c1[2H]]

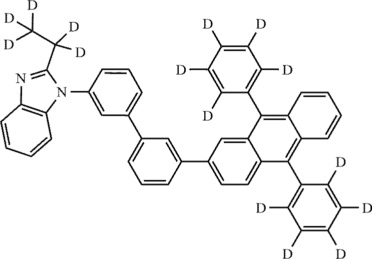 [SMILES: [2H]c1c([2H])c([2H])c(-c2c3ccccc3c(-c3c([2H])c([2H])c([2H])c([2H])c3[2H])c3cc(-c4cccc(-c5cccc(-n6c(C([2H])([2H])C([2H])([2H])[2H])nc7ccccc76)c5)c4)ccc23)c([2H])c1[2H]]

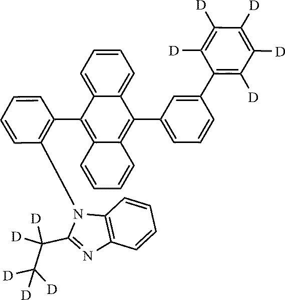 [SMILES: [2H]c1c([2H])c([2H])c(-c2cccc(-c3c4ccccc4c(-c4ccccc4-n4c(C([2H])([2H])C([2H])([2H])[2H])nc5ccccc54)c4ccccc34)c2)c([2H])c1[2H]]

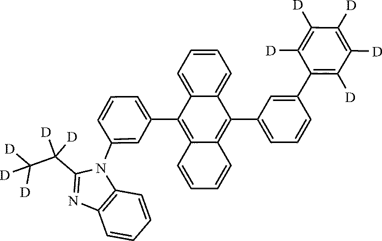 [SMILES: [2H]c1c([2H])c([2H])c(-c2cccc(-c3c4ccccc4c(-c4cccc(-n5c(C([2H])([2H])C([2H])([2H])[2H])nc6ccccc65)c4)c4ccccc34)c2)c([2H])c1[2H]]

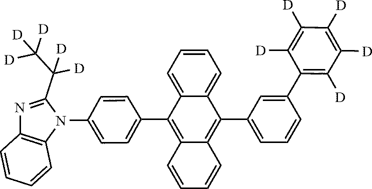 [SMILES: [2H]c1c([2H])c([2H])c(-c2cccc(-c3c4ccccc4c(-c4ccc(-n5c(C([2H])([2H])C([2H])([2H])[2H])nc6ccccc65)cc4)c4ccccc34)c2)c([2H])c1[2H]]

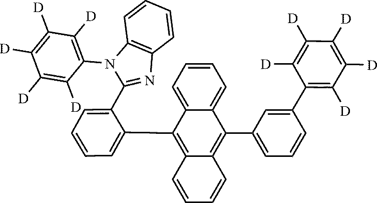 [SMILES: [2H]c1c([2H])c([2H])c(-c2cccc(-c3c4ccccc4c(-c4ccccc4-c4nc5ccccc5n4-c4c([2H])c([2H])c([2H])c([2H])c4[2H])c4ccccc34)c2)c([2H])c1[2H]]

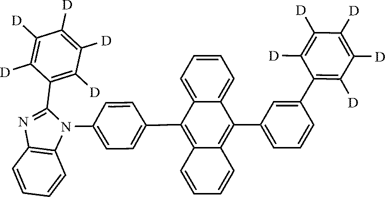 [SMILES: [2H]c1c([2H])c([2H])c(-c2cccc(-c3c4ccccc4c(-c4ccc(-n5c(-c6c([2H])c([2H])c([2H])c([2H])c6[2H])nc6ccccc65)cc4)c4ccccc34)c2)c([2H])c1[2H]]

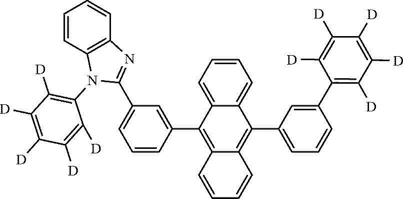 [SMILES: [2H]c1c([2H])c([2H])c(-c2cccc(-c3c4ccccc4c(-c4cccc(-c5nc6ccccc6n5-c5c([2H])c([2H])c([2H])c([2H])c5[2H])c4)c4ccccc34)c2)c([2H])c1[2H]]

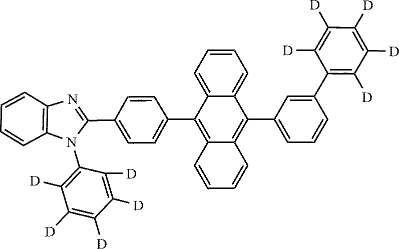 [SMILES: [2H]c1c([2H])c([2H])c(-c2cccc(-c3c4ccccc4c(-c4ccc(-c5nc6ccccc6n5-c5c([2H])c([2H])c([2H])c([2H])c5[2H])cc4)c4ccccc34)c2)c([2H])c1[2H]]

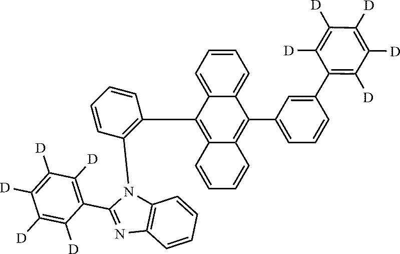 [SMILES: [2H]c1c([2H])c([2H])c(-c2cccc(-c3c4ccccc4c(-c4ccccc4-n4c(-c5c([2H])c([2H])c([2H])c([2H])c5[2H])nc5ccccc54)c4ccccc34)c2)c([2H])c1[2H]]

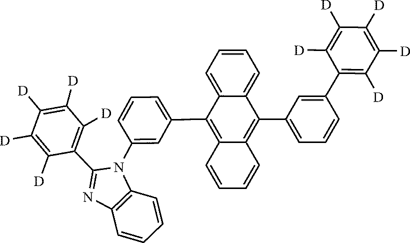 [SMILES: [2H]c1c([2H])c([2H])c(-c2cccc(-c3c4ccccc4c(-c4cccc(-n5c(-c6c([2H])c([2H])c([2H])c([2H])c6[2H])nc6ccccc65)c4)c4ccccc34)c2)c([2H])c1[2H]]

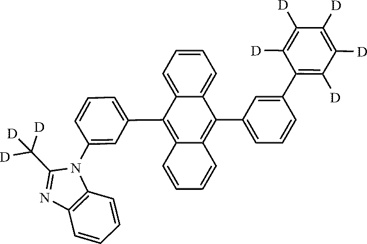 [SMILES: [2H]c1c([2H])c([2H])c(-c2cccc(-c3c4ccccc4c(-c4cccc(-n5c(C([2H])([2H])[2H])nc6ccccc65)c4)c4ccccc34)c2)c([2H])c1[2H]]

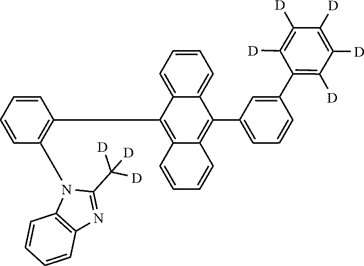 [SMILES: [2H]c1c([2H])c([2H])c(-c2cccc(-c3c4ccccc4c(-c4ccccc4-n4c(C([2H])([2H])[2H])nc5ccccc54)c4ccccc34)c2)c([2H])c1[2H]]

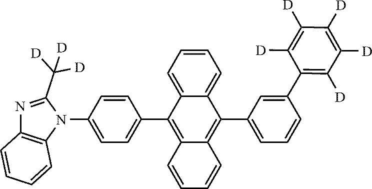 [SMILES: [2H]c1c([2H])c([2H])c(-c2cccc(-c3c4ccccc4c(-c4ccc(-n5c(C([2H])([2H])[2H])nc6ccccc65)cc4)c4ccccc34)c2)c([2H])c1[2H]]

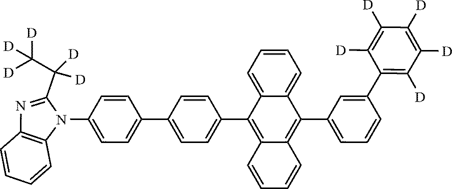 [SMILES: [2H]c1c([2H])c([2H])c(-c2cccc(-c3c4ccccc4c(-c4ccc(-c5ccc(-n6c(C([2H])([2H])C([2H])([2H])[2H])nc7ccccc76)cc5)cc4)c4ccccc34)c2)c([2H])c1[2H]]

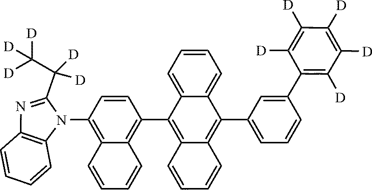 [SMILES: [2H]c1c([2H])c([2H])c(-c2cccc(-c3c4ccccc4c(-c4ccc(-n5c(C([2H])([2H])C([2H])([2H])[2H])nc6ccccc65)c5ccccc45)c4ccccc34)c2)c([2H])c1[2H]]

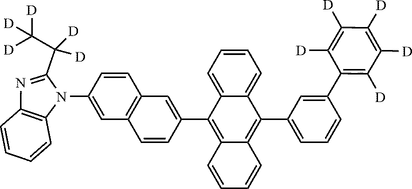 [SMILES: [2H]c1c([2H])c([2H])c(-c2cccc(-c3c4ccccc4c(-c4ccc5cc(-n6c(C([2H])([2H])C([2H])([2H])[2H])nc7ccccc76)ccc5c4)c4ccccc34)c2)c([2H])c1[2H]]

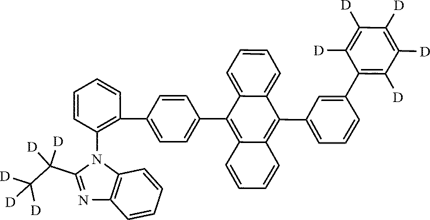 [SMILES: [2H]c1c([2H])c([2H])c(-c2cccc(-c3c4ccccc4c(-c4ccc(-c5ccccc5-n5c(C([2H])([2H])C([2H])([2H])[2H])nc6ccccc65)cc4)c4ccccc34)c2)c([2H])c1[2H]]

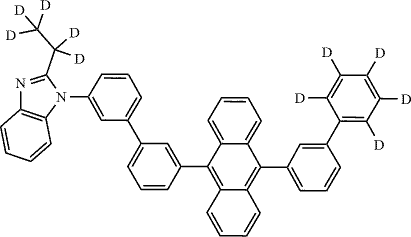 [SMILES: [2H]c1c([2H])c([2H])c(-c2cccc(-c3c4ccccc4c(-c4cccc(-c5cccc(-n6c(C([2H])([2H])C([2H])([2H])[2H])nc7ccccc76)c5)c4)c4ccccc34)c2)c([2H])c1[2H]]

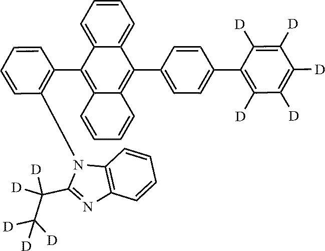 [SMILES: [2H]c1c([2H])c([2H])c(-c2ccc(-c3c4ccccc4c(-c4ccccc4-n4c(C([2H])([2H])C([2H])([2H])[2H])nc5ccccc54)c4ccccc34)cc2)c([2H])c1[2H]]

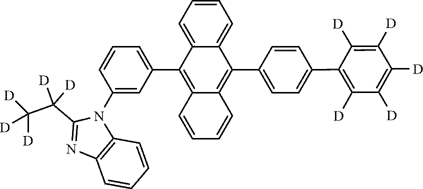 [SMILES: [2H]c1c([2H])c([2H])c(-c2ccc(-c3c4ccccc4c(-c4cccc(-n5c(C([2H])([2H])C([2H])([2H])[2H])nc6ccccc65)c4)c4ccccc34)cc2)c([2H])c1[2H]]

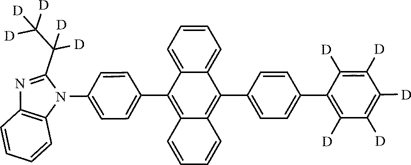 [SMILES: [2H]c1c([2H])c([2H])c(-c2ccc(-c3c4ccccc4c(-c4ccc(-n5c(C([2H])([2H])C([2H])([2H])[2H])nc6ccccc65)cc4)c4ccccc34)cc2)c([2H])c1[2H]]

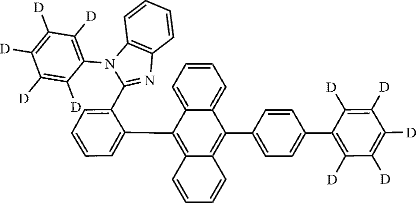 [SMILES: [2H]c1c([2H])c([2H])c(-c2ccc(-c3c4ccccc4c(-c4ccccc4-c4nc5ccccc5n4-c4c([2H])c([2H])c([2H])c([2H])c4[2H])c4ccccc34)cc2)c([2H])c1[2H]]

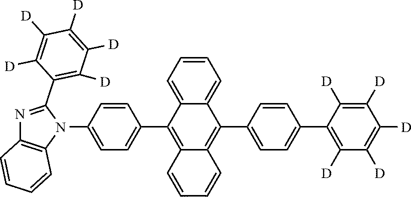 [SMILES: [2H]c1c([2H])c([2H])c(-c2ccc(-c3c4ccccc4c(-c4ccc(-n5c(-c6c([2H])c([2H])c([2H])c([2H])c6[2H])nc6ccccc65)cc4)c4ccccc34)cc2)c([2H])c1[2H]]

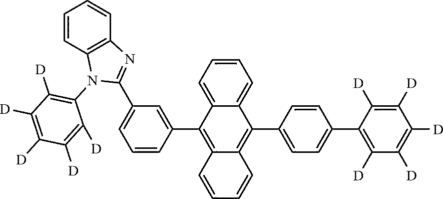 [SMILES: [2H]c1c([2H])c([2H])c(-c2ccc(-c3c4ccccc4c(-c4cccc(-c5nc6ccccc6n5-c5c([2H])c([2H])c([2H])c([2H])c5[2H])c4)c4ccccc34)cc2)c([2H])c1[2H]]

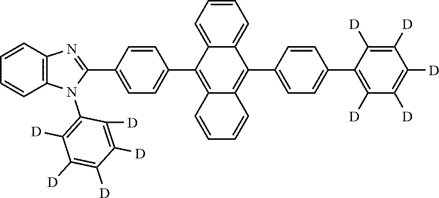 [SMILES: [2H]c1c([2H])c([2H])c(-c2ccc(-c3c4ccccc4c(-c4ccc(-c5nc6ccccc6n5-c5c([2H])c([2H])c([2H])c([2H])c5[2H])cc4)c4ccccc34)cc2)c([2H])c1[2H]]

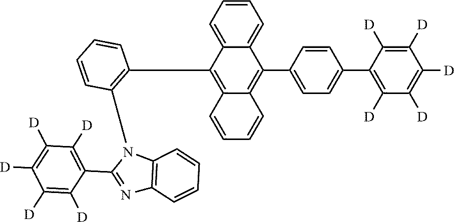 [SMILES: [2H]c1c([2H])c([2H])c(-c2ccc(-c3c4ccccc4c(-c4ccccc4-n4c(-c5c([2H])c([2H])c([2H])c([2H])c5[2H])nc5ccccc54)c4ccccc34)cc2)c([2H])c1[2H]]

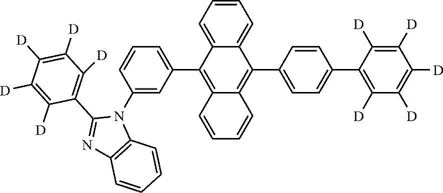 [SMILES: [2H]c1c([2H])c([2H])c(-c2ccc(-c3c4ccccc4c(-c4cccc(-n5c(-c6c([2H])c([2H])c([2H])c([2H])c6[2H])nc6ccccc65)c4)c4ccccc34)cc2)c([2H])c1[2H]]

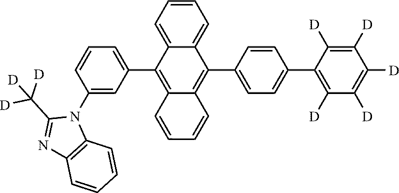 [SMILES: [2H]c1c([2H])c([2H])c(-c2ccc(-c3c4ccccc4c(-c4cccc(-n5c(C([2H])([2H])[2H])nc6ccccc65)c4)c4ccccc34)cc2)c([2H])c1[2H]]

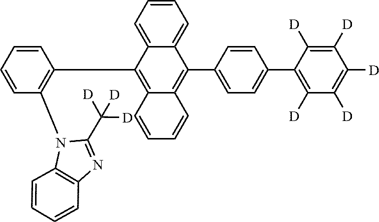 [SMILES: [2H]c1c([2H])c([2H])c(-c2ccc(-c3c4ccccc4c(-c4ccccc4-n4c(C([2H])([2H])[2H])nc5ccccc54)c4ccccc34)cc2)c([2H])c1[2H]]

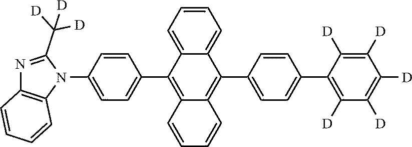 [SMILES: [2H]c1c([2H])c([2H])c(-c2ccc(-c3c4ccccc4c(-c4ccc(-n5c(C([2H])([2H])[2H])nc6ccccc65)cc4)c4ccccc34)cc2)c([2H])c1[2H]]

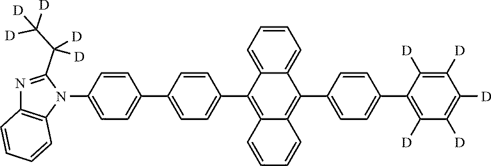 [SMILES: [2H]c1c([2H])c([2H])c(-c2ccc(-c3c4ccccc4c(-c4ccc(-c5ccc(-n6c(C([2H])([2H])C([2H])([2H])[2H])nc7ccccc76)cc5)cc4)c4ccccc34)cc2)c([2H])c1[2H]]

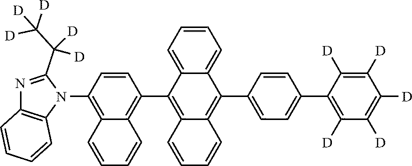 [SMILES: [2H]c1c([2H])c([2H])c(-c2ccc(-c3c4ccccc4c(-c4ccc(-n5c(C([2H])([2H])C([2H])([2H])[2H])nc6ccccc65)c5ccccc45)c4ccccc34)cc2)c([2H])c1[2H]]

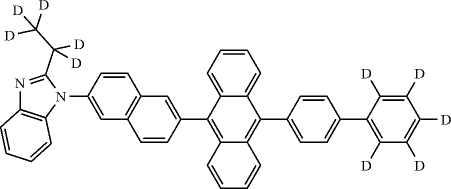 [SMILES: [2H]c1c([2H])c([2H])c(-c2ccc(-c3c4ccccc4c(-c4ccc5cc(-n6c(C([2H])([2H])C([2H])([2H])[2H])nc7ccccc76)ccc5c4)c4ccccc34)cc2)c([2H])c1[2H]]

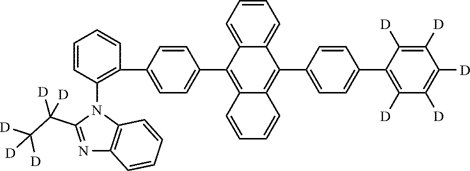 [SMILES: [2H]c1c([2H])c([2H])c(-c2ccc(-c3c4ccccc4c(-c4ccc(-c5ccccc5-n5c(C([2H])([2H])C([2H])([2H])[2H])nc6ccccc65)cc4)c4ccccc34)cc2)c([2H])c1[2H]]

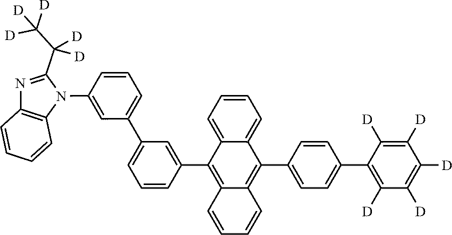 [SMILES: [2H]c1c([2H])c([2H])c(-c2ccc(-c3c4ccccc4c(-c4cccc(-c5cccc(-n6c(C([2H])([2H])C([2H])([2H])[2H])nc7ccccc76)c5)c4)c4ccccc34)cc2)c([2H])c1[2H]]

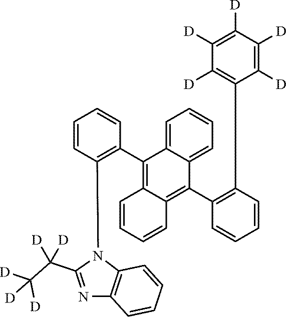 [SMILES: [2H]c1c([2H])c([2H])c(-c2ccccc2-c2c3ccccc3c(-c3ccccc3-n3c(C([2H])([2H])C([2H])([2H])[2H])nc4ccccc43)c3ccccc23)c([2H])c1[2H]]

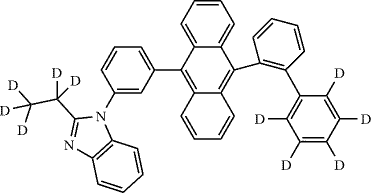 [SMILES: [2H]c1c([2H])c([2H])c(-c2ccccc2-c2c3ccccc3c(-c3cccc(-n4c(C([2H])([2H])C([2H])([2H])[2H])nc5ccccc54)c3)c3ccccc23)c([2H])c1[2H]]

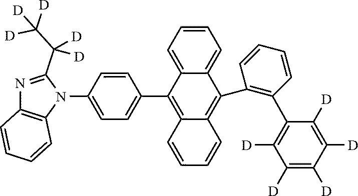 [SMILES: [2H]c1c([2H])c([2H])c(-c2ccccc2-c2c3ccccc3c(-c3ccc(-n4c(C([2H])([2H])C([2H])([2H])[2H])nc5ccccc54)cc3)c3ccccc23)c([2H])c1[2H]]

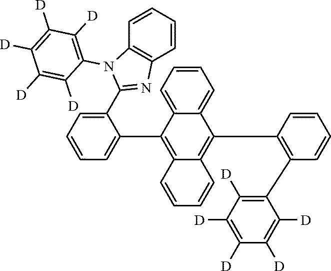 [SMILES: [2H]c1c([2H])c([2H])c(-c2ccccc2-c2c3ccccc3c(-c3ccccc3-c3nc4ccccc4n3-c3c([2H])c([2H])c([2H])c([2H])c3[2H])c3ccccc23)c([2H])c1[2H]]

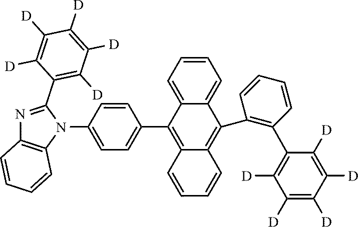 [SMILES: [2H]c1c([2H])c([2H])c(-c2ccccc2-c2c3ccccc3c(-c3ccc(-n4c(-c5c([2H])c([2H])c([2H])c([2H])c5[2H])nc5ccccc54)cc3)c3ccccc23)c([2H])c1[2H]]

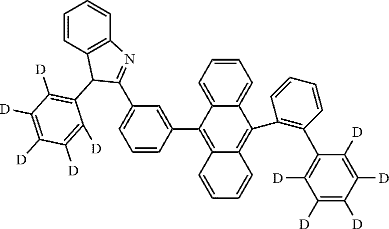 [SMILES: [2H]c1c([2H])c([2H])c(-c2ccccc2-c2c3ccccc3c(-c3cccc(C4=Nc5ccccc5C4c4c([2H])c([2H])c([2H])c([2H])c4[2H])c3)c3ccccc23)c([2H])c1[2H]]

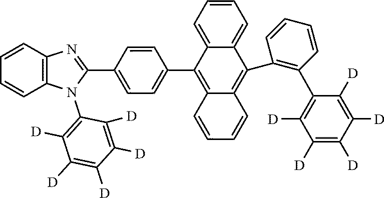 [SMILES: [2H]c1c([2H])c([2H])c(-c2ccccc2-c2c3ccccc3c(-c3ccc(-c4nc5ccccc5n4-c4c([2H])c([2H])c([2H])c([2H])c4[2H])cc3)c3ccccc23)c([2H])c1[2H]]

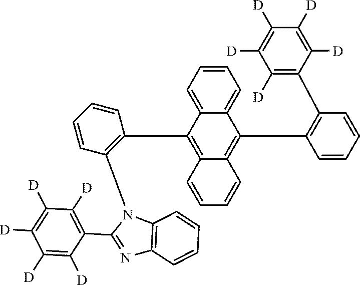 [SMILES: [2H]c1c([2H])c([2H])c(-c2ccccc2-c2c3ccccc3c(-c3ccccc3-n3c(-c4c([2H])c([2H])c([2H])c([2H])c4[2H])nc4ccccc43)c3ccccc23)c([2H])c1[2H]]

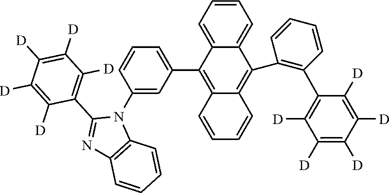 [SMILES: [2H]c1c([2H])c([2H])c(-c2ccccc2-c2c3ccccc3c(-c3cccc(-n4c(-c5c([2H])c([2H])c([2H])c([2H])c5[2H])nc5ccccc54)c3)c3ccccc23)c([2H])c1[2H]]

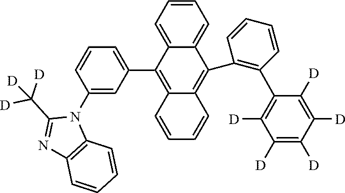 [SMILES: [2H]c1c([2H])c([2H])c(-c2ccccc2-c2c3ccccc3c(-c3cccc(-n4c(C([2H])([2H])[2H])nc5ccccc54)c3)c3ccccc23)c([2H])c1[2H]]

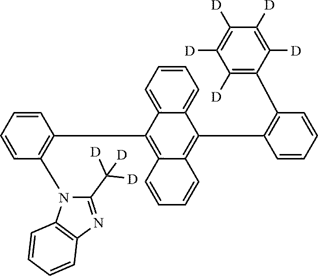 [SMILES: [2H]c1c([2H])c([2H])c(-c2ccccc2-c2c3ccccc3c(-c3ccccc3-n3c(C([2H])([2H])[2H])nc4ccccc43)c3ccccc23)c([2H])c1[2H]]

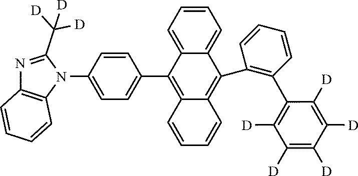 [SMILES: [2H]c1c([2H])c([2H])c(-c2ccccc2-c2c3ccccc3c(-c3ccc(-n4c(C([2H])([2H])[2H])nc5ccccc54)cc3)c3ccccc23)c([2H])c1[2H]]

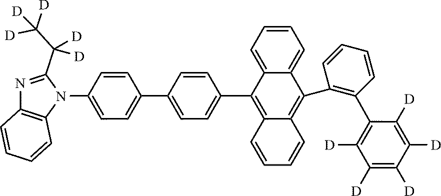 [SMILES: [2H]c1c([2H])c([2H])c(-c2ccccc2-c2c3ccccc3c(-c3ccc(-c4ccc(-n5c(C([2H])([2H])C([2H])([2H])[2H])nc6ccccc65)cc4)cc3)c3ccccc23)c([2H])c1[2H]]

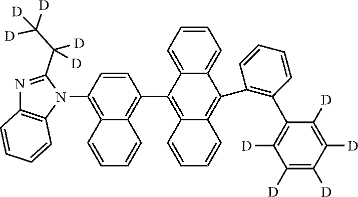 [SMILES: [2H]c1c([2H])c([2H])c(-c2ccccc2-c2c3ccccc3c(-c3ccc(-n4c(C([2H])([2H])C([2H])([2H])[2H])nc5ccccc54)c4ccccc34)c3ccccc23)c([2H])c1[2H]]

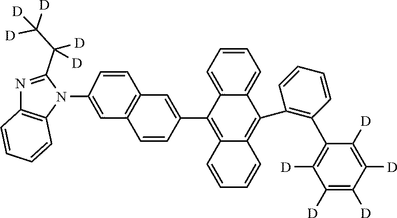 [SMILES: [2H]c1c([2H])c([2H])c(-c2ccccc2-c2c3ccccc3c(-c3ccc4cc(-n5c(C([2H])([2H])C([2H])([2H])[2H])nc6ccccc65)ccc4c3)c3ccccc23)c([2H])c1[2H]]

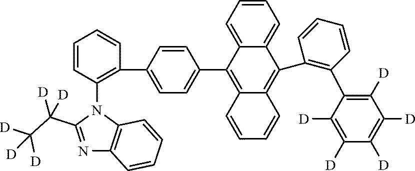 [SMILES: [2H]c1c([2H])c([2H])c(-c2ccccc2-c2c3ccccc3c(-c3ccc(-c4ccccc4-n4c(C([2H])([2H])C([2H])([2H])[2H])nc5ccccc54)cc3)c3ccccc23)c([2H])c1[2H]]

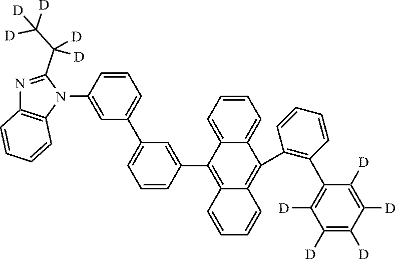 [SMILES: [2H]c1c([2H])c([2H])c(-c2ccccc2-c2c3ccccc3c(-c3cccc(-c4cccc(-n5c(C([2H])([2H])C([2H])([2H])[2H])nc6ccccc65)c4)c3)c3ccccc23)c([2H])c1[2H]]

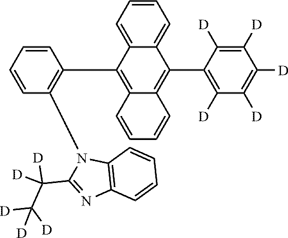 [SMILES: [2H]c1c([2H])c([2H])c(-c2c3ccccc3c(-c3ccccc3-n3c(C([2H])([2H])C([2H])([2H])[2H])nc4ccccc43)c3ccccc23)c([2H])c1[2H]]

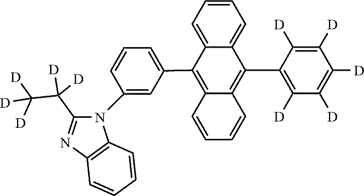 [SMILES: [2H]c1c([2H])c([2H])c(-c2c3ccccc3c(-c3cccc(-n4c(C([2H])([2H])C([2H])([2H])[2H])nc5ccccc54)c3)c3ccccc23)c([2H])c1[2H]]

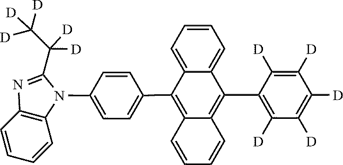 [SMILES: [2H]c1c([2H])c([2H])c(-c2c3ccccc3c(-c3ccc(-n4c(C([2H])([2H])C([2H])([2H])[2H])nc5ccccc54)cc3)c3ccccc23)c([2H])c1[2H]]

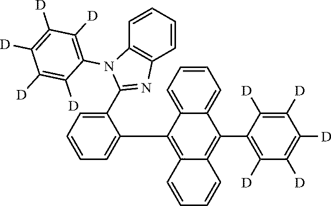 [SMILES: [2H]c1c([2H])c([2H])c(-c2c3ccccc3c(-c3ccccc3-c3nc4ccccc4n3-c3c([2H])c([2H])c([2H])c([2H])c3[2H])c3ccccc23)c([2H])c1[2H]]

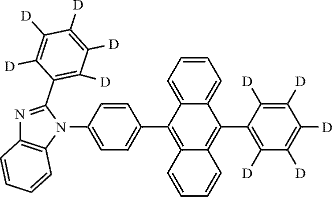 [SMILES: [2H]c1c([2H])c([2H])c(-c2c3ccccc3c(-c3ccc(-n4c(-c5c([2H])c([2H])c([2H])c([2H])c5[2H])nc5ccccc54)cc3)c3ccccc23)c([2H])c1[2H]]

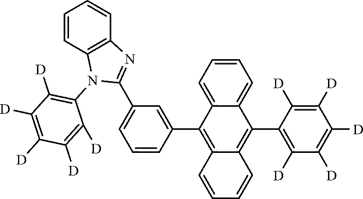 [SMILES: [2H]c1c([2H])c([2H])c(-c2c3ccccc3c(-c3cccc(-c4nc5ccccc5n4-c4c([2H])c([2H])c([2H])c([2H])c4[2H])c3)c3ccccc23)c([2H])c1[2H]]

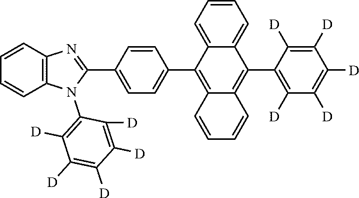 [SMILES: [2H]c1c([2H])c([2H])c(-c2c3ccccc3c(-c3ccc(-c4nc5ccccc5n4-c4c([2H])c([2H])c([2H])c([2H])c4[2H])cc3)c3ccccc23)c([2H])c1[2H]]

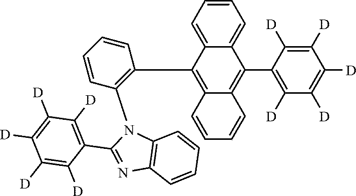 [SMILES: [2H]c1c([2H])c([2H])c(-c2c3ccccc3c(-c3ccccc3-n3c(-c4c([2H])c([2H])c([2H])c([2H])c4[2H])nc4ccccc43)c3ccccc23)c([2H])c1[2H]]

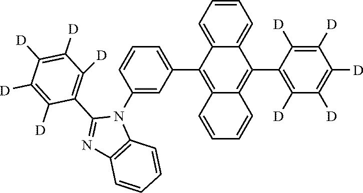 [SMILES: [2H]c1c([2H])c([2H])c(-c2c3ccccc3c(-c3cccc(-n4c(-c5c([2H])c([2H])c([2H])c([2H])c5[2H])nc5ccccc54)c3)c3ccccc23)c([2H])c1[2H]]

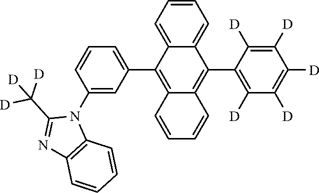 [SMILES: [2H]c1c([2H])c([2H])c(-c2c3ccccc3c(-c3cccc(-n4c(C([2H])([2H])[2H])nc5ccccc54)c3)c3ccccc23)c([2H])c1[2H]]

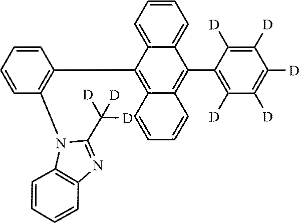 [SMILES: [2H]c1c([2H])c([2H])c(-c2c3ccccc3c(-c3ccccc3-n3c(C([2H])([2H])[2H])nc4ccccc43)c3ccccc23)c([2H])c1[2H]]

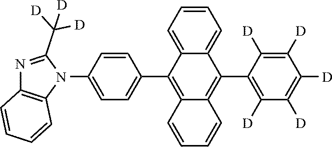 [SMILES: [2H]c1c([2H])c([2H])c(-c2c3ccccc3c(-c3ccc(-n4c(C([2H])([2H])[2H])nc5ccccc54)cc3)c3ccccc23)c([2H])c1[2H]]

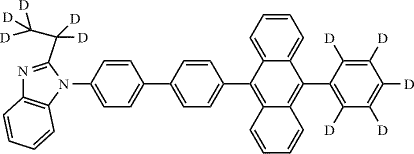 [SMILES: [2H]c1c([2H])c([2H])c(-c2c3ccccc3c(-c3ccc(-c4ccc(-n5c(C([2H])([2H])C([2H])([2H])[2H])nc6ccccc65)cc4)cc3)c3ccccc23)c([2H])c1[2H]]

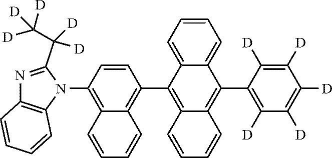 [SMILES: [2H]c1c([2H])c([2H])c(-c2c3ccccc3c(-c3ccc(-n4c(C([2H])([2H])C([2H])([2H])[2H])nc5ccccc54)c4ccccc34)c3ccccc23)c([2H])c1[2H]]

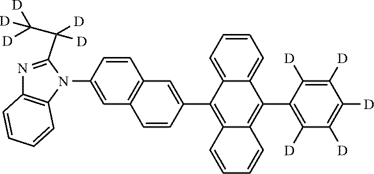 [SMILES: [2H]c1c([2H])c([2H])c(-c2c3ccccc3c(-c3ccc4cc(-n5c(C([2H])([2H])C([2H])([2H])[2H])nc6ccccc65)ccc4c3)c3ccccc23)c([2H])c1[2H]]

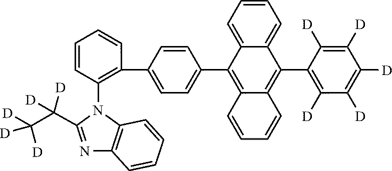 [SMILES: [2H]c1c([2H])c([2H])c(-c2c3ccccc3c(-c3ccc(-c4ccccc4-n4c(C([2H])([2H])C([2H])([2H])[2H])nc5ccccc54)cc3)c3ccccc23)c([2H])c1[2H]]

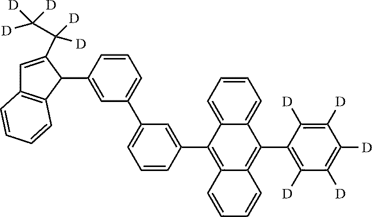 [SMILES: [2H]c1c([2H])c([2H])c(-c2c3ccccc3c(-c3cccc(-c4cccc(C5C(C([2H])([2H])C([2H])([2H])[2H])=Cc6ccccc65)c4)c3)c3ccccc23)c([2H])c1[2H]]

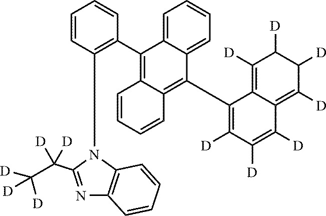 [SMILES: [2H]C1=c2c([2H])c([2H])c([2H])c(-c3c4ccccc4c(-c4ccccc4-n4c(C([2H])([2H])C([2H])([2H])[2H])nc5ccccc54)c4ccccc34)c2=C([2H])C([2H])C1[2H]]

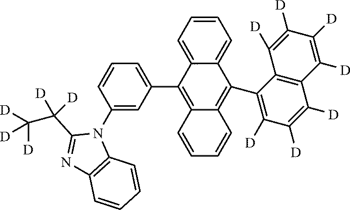 [SMILES: [2H]c1c([2H])c([2H])c2c(-c3c4ccccc4c(-c4cccc(-n5c(C([2H])([2H])C([2H])([2H])[2H])nc6ccccc65)c4)c4ccccc34)c([2H])c([2H])c([2H])c2c1[2H]]

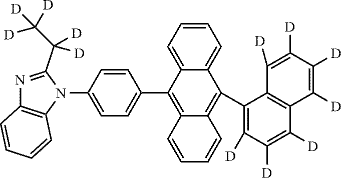 [SMILES: [2H]c1c([2H])c([2H])c2c(-c3c4ccccc4c(-c4ccc(-n5c(C([2H])([2H])C([2H])([2H])[2H])nc6ccccc65)cc4)c4ccccc34)c([2H])c([2H])c([2H])c2c1[2H]]

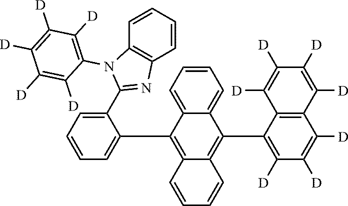 [SMILES: [2H]c1c([2H])c([2H])c(-n2c(-c3ccccc3-c3c4ccccc4c(-c4c([2H])c([2H])c([2H])c5c([2H])c([2H])c([2H])c([2H])c45)c4ccccc34)nc3ccccc32)c([2H])c1[2H]]

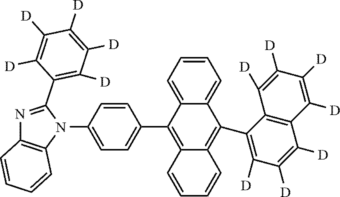 [SMILES: [2H]c1c([2H])c([2H])c(-c2nc3ccccc3n2-c2ccc(-c3c4ccccc4c(-c4c([2H])c([2H])c([2H])c5c([2H])c([2H])c([2H])c([2H])c45)c4ccccc34)cc2)c([2H])c1[2H]]

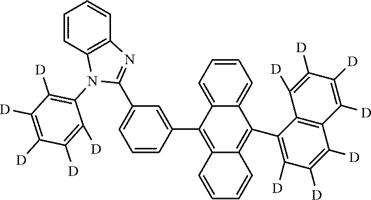 [SMILES: [2H]c1c([2H])c([2H])c(-n2c(-c3cccc(-c4c5ccccc5c(-c5c([2H])c([2H])c([2H])c6c([2H])c([2H])c([2H])c([2H])c56)c5ccccc45)c3)nc3ccccc32)c([2H])c1[2H]]

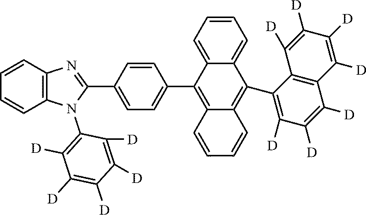 [SMILES: [2H]c1c([2H])c([2H])c(-n2c(-c3ccc(-c4c5ccccc5c(-c5c([2H])c([2H])c([2H])c6c([2H])c([2H])c([2H])c([2H])c56)c5ccccc45)cc3)nc3ccccc32)c([2H])c1[2H]]

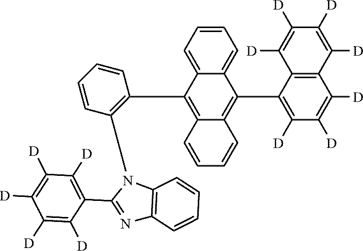 [SMILES: [2H]c1c([2H])c([2H])c(-c2nc3ccccc3n2-c2ccccc2-c2c3ccccc3c(-c3c([2H])c([2H])c([2H])c4c([2H])c([2H])c([2H])c([2H])c34)c3ccccc23)c([2H])c1[2H]]

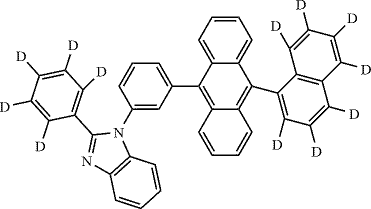 [SMILES: [2H]c1c([2H])c([2H])c(-c2nc3ccccc3n2-c2cccc(-c3c4ccccc4c(-c4c([2H])c([2H])c([2H])c5c([2H])c([2H])c([2H])c([2H])c45)c4ccccc34)c2)c([2H])c1[2H]]

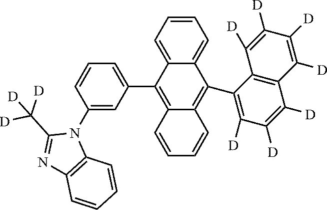 [SMILES: [2H]c1c([2H])c([2H])c2c(-c3c4ccccc4c(-c4cccc(-n5c(C([2H])([2H])[2H])nc6ccccc65)c4)c4ccccc34)c([2H])c([2H])c([2H])c2c1[2H]]

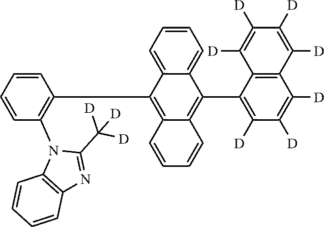 [SMILES: [2H]c1c([2H])c([2H])c2c(-c3c4ccccc4c(-c4ccccc4-n4c(C([2H])([2H])[2H])nc5ccccc54)c4ccccc34)c([2H])c([2H])c([2H])c2c1[2H]]

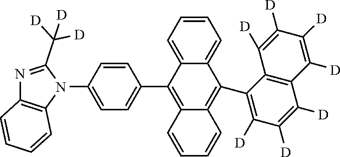 [SMILES: [2H]c1c([2H])c([2H])c2c(-c3c4ccccc4c(-c4ccc(-n5c(C([2H])([2H])[2H])nc6ccccc65)cc4)c4ccccc34)c([2H])c([2H])c([2H])c2c1[2H]]

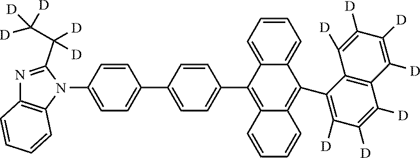 [SMILES: [2H]c1c([2H])c([2H])c2c(-c3c4ccccc4c(-c4ccc(-c5ccc(-n6c(C([2H])([2H])C([2H])([2H])[2H])nc7ccccc76)cc5)cc4)c4ccccc34)c([2H])c([2H])c([2H])c2c1[2H]]

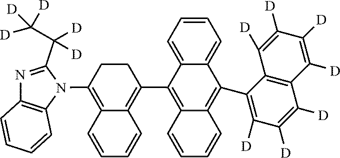 [SMILES: [2H]c1c([2H])c([2H])c2c(-c3c4ccccc4c(C4=c5ccccc5=C(n5c(C([2H])([2H])C([2H])([2H])[2H])nc6ccccc65)CC4)c4ccccc34)c([2H])c([2H])c([2H])c2c1[2H]]

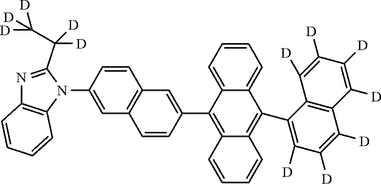 [SMILES: [2H]c1c([2H])c([2H])c2c(-c3c4ccccc4c(-c4ccc5cc(-n6c(C([2H])([2H])C([2H])([2H])[2H])nc7ccccc76)ccc5c4)c4ccccc34)c([2H])c([2H])c([2H])c2c1[2H]]

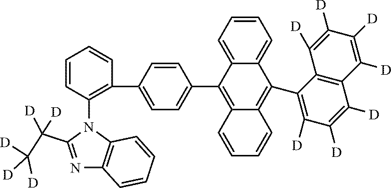 [SMILES: [2H]c1c([2H])c([2H])c2c(-c3c4ccccc4c(-c4ccc(-c5ccccc5-n5c(C([2H])([2H])C([2H])([2H])[2H])nc6ccccc65)cc4)c4ccccc34)c([2H])c([2H])c([2H])c2c1[2H]]

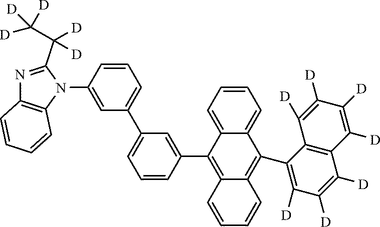 [SMILES: [2H]c1c([2H])c([2H])c2c(-c3c4ccccc4c(-c4cccc(-c5cccc(-n6c(C([2H])([2H])C([2H])([2H])[2H])nc7ccccc76)c5)c4)c4ccccc34)c([2H])c([2H])c([2H])c2c1[2H]]

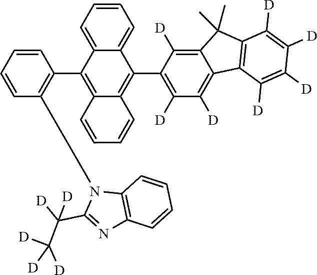 [SMILES: [2H]c1c([2H])c([2H])c2c(c1[2H])-c1c([2H])c([2H])c(-c3c4ccccc4c(-c4ccccc4-n4c(C([2H])([2H])C([2H])([2H])[2H])nc5ccccc54)c4ccccc34)c([2H])c1C2(C)C]

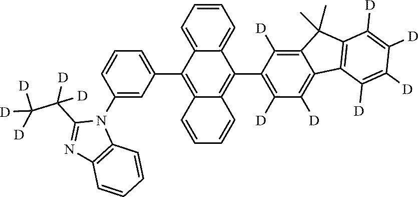 [SMILES: [2H]c1c([2H])c([2H])c2c(c1[2H])-c1c([2H])c([2H])c(-c3c4ccccc4c(-c4cccc(-n5c(C([2H])([2H])C([2H])([2H])[2H])nc6ccccc65)c4)c4ccccc34)c([2H])c1C2(C)C]

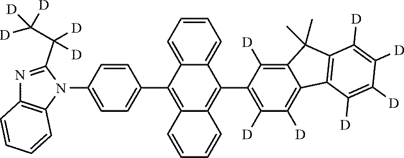 [SMILES: [2H]c1c([2H])c([2H])c2c(c1[2H])-c1c([2H])c([2H])c(-c3c4ccccc4c(-c4ccc(-n5c(C([2H])([2H])C([2H])([2H])[2H])nc6ccccc65)cc4)c4ccccc34)c([2H])c1C2(C)C]

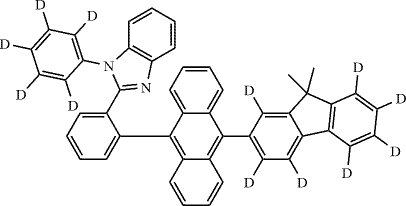 [SMILES: [2H]c1c([2H])c([2H])c(-n2c(-c3ccccc3-c3c4ccccc4c(-c4c([2H])c([2H])c5c(c4[2H])C(C)(C)c4c([2H])c([2H])c([2H])c([2H])c4-5)c4ccccc34)nc3ccccc32)c([2H])c1[2H]]

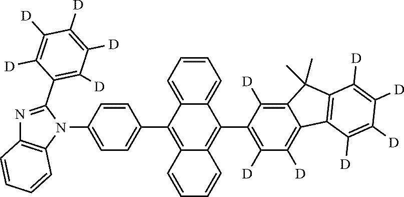 [SMILES: [2H]c1c([2H])c([2H])c(-c2nc3ccccc3n2-c2ccc(-c3c4ccccc4c(-c4c([2H])c([2H])c5c(c4[2H])C(C)(C)c4c([2H])c([2H])c([2H])c([2H])c4-5)c4ccccc34)cc2)c([2H])c1[2H]]

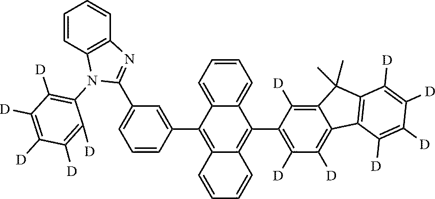 [SMILES: [2H]c1c([2H])c([2H])c(-n2c(-c3cccc(-c4c5ccccc5c(-c5c([2H])c([2H])c6c(c5[2H])C(C)(C)c5c([2H])c([2H])c([2H])c([2H])c5-6)c5ccccc45)c3)nc3ccccc32)c([2H])c1[2H]]

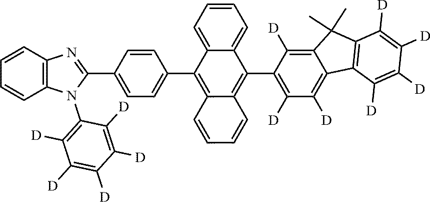 [SMILES: [2H]c1c([2H])c([2H])c(-n2c(-c3ccc(-c4c5ccccc5c(-c5c([2H])c([2H])c6c(c5[2H])C(C)(C)c5c([2H])c([2H])c([2H])c([2H])c5-6)c5ccccc45)cc3)nc3ccccc32)c([2H])c1[2H]]

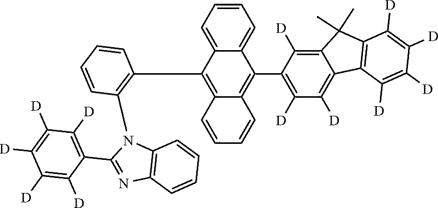 [SMILES: [2H]c1c([2H])c([2H])c(-c2nc3ccccc3n2-c2ccccc2-c2c3ccccc3c(-c3c([2H])c([2H])c4c(c3[2H])C(C)(C)c3c([2H])c([2H])c([2H])c([2H])c3-4)c3ccccc23)c([2H])c1[2H]]

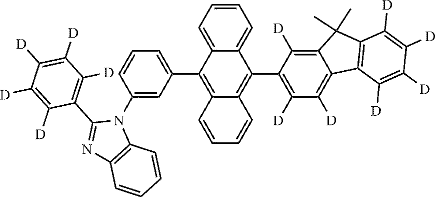 [SMILES: [2H]c1c([2H])c([2H])c(-c2nc3ccccc3n2-c2cccc(-c3c4ccccc4c(-c4c([2H])c([2H])c5c(c4[2H])C(C)(C)c4c([2H])c([2H])c([2H])c([2H])c4-5)c4ccccc34)c2)c([2H])c1[2H]]

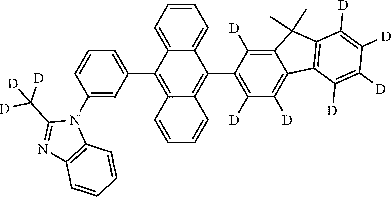 [SMILES: [2H]c1c([2H])c([2H])c2c(c1[2H])-c1c([2H])c([2H])c(-c3c4ccccc4c(-c4cccc(-n5c(C([2H])([2H])[2H])nc6ccccc65)c4)c4ccccc34)c([2H])c1C2(C)C]

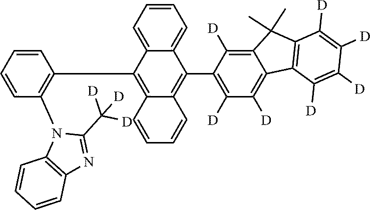 [SMILES: [2H]c1c([2H])c([2H])c2c(c1[2H])-c1c([2H])c([2H])c(-c3c4ccccc4c(-c4ccccc4-n4c(C([2H])([2H])[2H])nc5ccccc54)c4ccccc34)c([2H])c1C2(C)C]

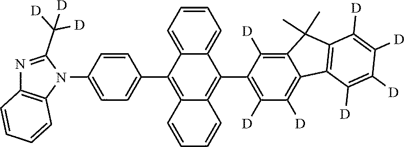 [SMILES: [2H]c1c([2H])c([2H])c2c(c1[2H])-c1c([2H])c([2H])c(-c3c4ccccc4c(-c4ccc(-n5c(C([2H])([2H])[2H])nc6ccccc65)cc4)c4ccccc34)c([2H])c1C2(C)C]

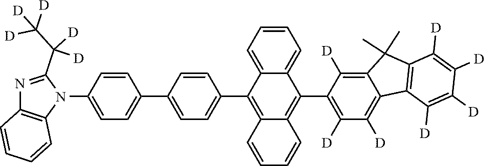 [SMILES: [2H]c1c([2H])c([2H])c2c(c1[2H])-c1c([2H])c([2H])c(-c3c4ccccc4c(-c4ccc(-c5ccc(-n6c(C([2H])([2H])C([2H])([2H])[2H])nc7ccccc76)cc5)cc4)c4ccccc34)c([2H])c1C2(C)C]

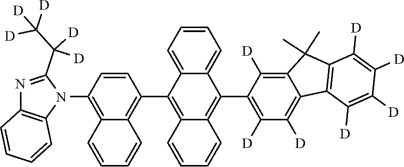 [SMILES: [2H]c1c([2H])c([2H])c2c(c1[2H])-c1c([2H])c([2H])c(-c3c4ccccc4c(-c4ccc(-n5c(C([2H])([2H])C([2H])([2H])[2H])nc6ccccc65)c5ccccc45)c4ccccc34)c([2H])c1C2(C)C]

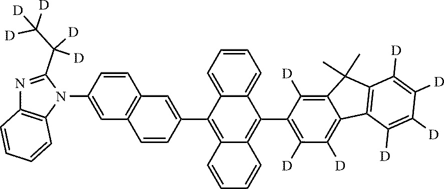 [SMILES: [2H]c1c([2H])c([2H])c2c(c1[2H])-c1c([2H])c([2H])c(-c3c4ccccc4c(-c4ccc5cc(-n6c(C([2H])([2H])C([2H])([2H])[2H])nc7ccccc76)ccc5c4)c4ccccc34)c([2H])c1C2(C)C]

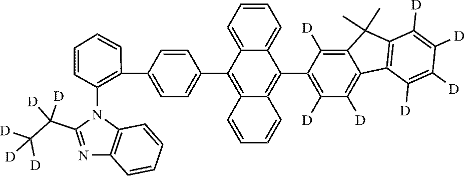 [SMILES: [2H]c1c([2H])c([2H])c2c(c1[2H])-c1c([2H])c([2H])c(-c3c4ccccc4c(-c4ccc(-c5ccccc5-n5c(C([2H])([2H])C([2H])([2H])[2H])nc6ccccc65)cc4)c4ccccc34)c([2H])c1C2(C)C]

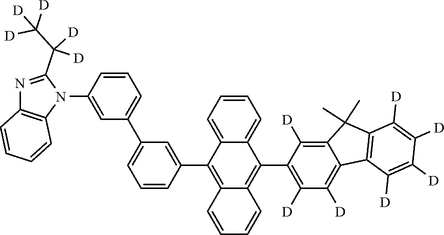 [SMILES: [2H]c1c([2H])c([2H])c2c(c1[2H])-c1c([2H])c([2H])c(-c3c4ccccc4c(-c4cccc(-c5cccc(-n6c(C([2H])([2H])C([2H])([2H])[2H])nc7ccccc76)c5)c4)c4ccccc34)c([2H])c1C2(C)C]

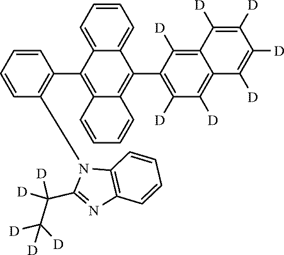 [SMILES: [2H]c1c([2H])c([2H])c2c([2H])c(-c3c4ccccc4c(-c4ccccc4-n4c(C([2H])([2H])C([2H])([2H])[2H])nc5ccccc54)c4ccccc34)c([2H])c([2H])c2c1[2H]]

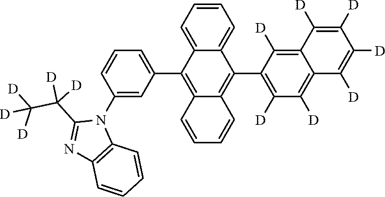 [SMILES: [2H]c1c([2H])c([2H])c2c([2H])c(-c3c4ccccc4c(-c4cccc(-n5c(C([2H])([2H])C([2H])([2H])[2H])nc6ccccc65)c4)c4ccccc34)c([2H])c([2H])c2c1[2H]]

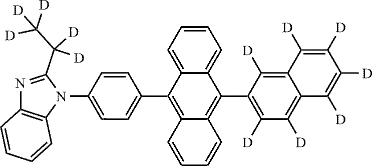 [SMILES: [2H]c1c([2H])c([2H])c2c([2H])c(-c3c4ccccc4c(-c4ccc(-n5c(C([2H])([2H])C([2H])([2H])[2H])nc6ccccc65)cc4)c4ccccc34)c([2H])c([2H])c2c1[2H]]

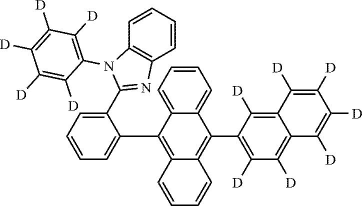 [SMILES: [2H]c1c([2H])c([2H])c(-n2c(-c3ccccc3-c3c4ccccc4c(-c4c([2H])c([2H])c5c([2H])c([2H])c([2H])c([2H])c5c4[2H])c4ccccc34)nc3ccccc32)c([2H])c1[2H]]

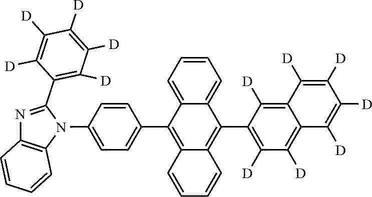 [SMILES: [2H]c1c([2H])c([2H])c(-c2nc3ccccc3n2-c2ccc(-c3c4ccccc4c(-c4c([2H])c([2H])c5c([2H])c([2H])c([2H])c([2H])c5c4[2H])c4ccccc34)cc2)c([2H])c1[2H]]

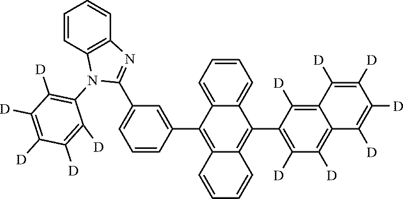 [SMILES: [2H]c1c([2H])c([2H])c(-n2c(-c3cccc(-c4c5ccccc5c(-c5c([2H])c([2H])c6c([2H])c([2H])c([2H])c([2H])c6c5[2H])c5ccccc45)c3)nc3ccccc32)c([2H])c1[2H]]

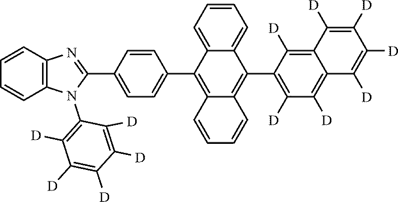 [SMILES: [2H]c1c([2H])c([2H])c(-n2c(-c3ccc(-c4c5ccccc5c(-c5c([2H])c([2H])c6c([2H])c([2H])c([2H])c([2H])c6c5[2H])c5ccccc45)cc3)nc3ccccc32)c([2H])c1[2H]]

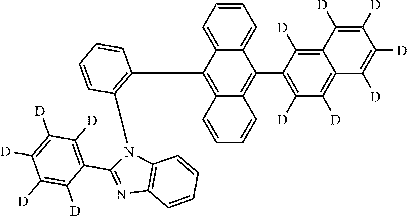 [SMILES: [2H]c1c([2H])c([2H])c(-c2nc3ccccc3n2-c2ccccc2-c2c3ccccc3c(-c3c([2H])c([2H])c4c([2H])c([2H])c([2H])c([2H])c4c3[2H])c3ccccc23)c([2H])c1[2H]]

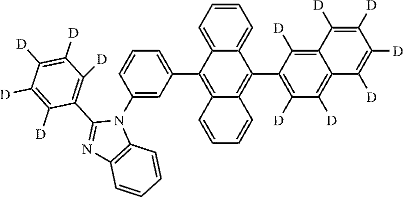 [SMILES: [2H]c1c([2H])c([2H])c(-c2nc3ccccc3n2-c2cccc(-c3c4ccccc4c(-c4c([2H])c([2H])c5c([2H])c([2H])c([2H])c([2H])c5c4[2H])c4ccccc34)c2)c([2H])c1[2H]]

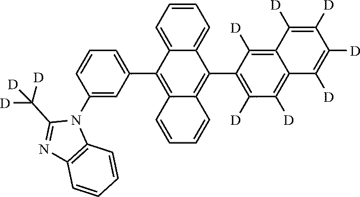 [SMILES: [2H]c1c([2H])c([2H])c2c([2H])c(-c3c4ccccc4c(-c4cccc(-n5c(C([2H])([2H])[2H])nc6ccccc65)c4)c4ccccc34)c([2H])c([2H])c2c1[2H]]

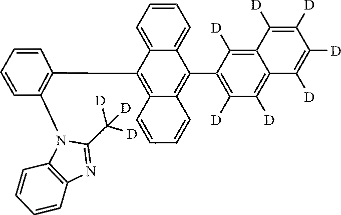 [SMILES: [2H]c1c([2H])c([2H])c2c([2H])c(-c3c4ccccc4c(-c4ccccc4-n4c(C([2H])([2H])[2H])nc5ccccc54)c4ccccc34)c([2H])c([2H])c2c1[2H]]

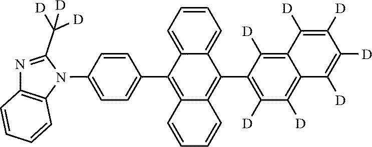 [SMILES: [2H]c1c([2H])c([2H])c2c([2H])c(-c3c4ccccc4c(-c4ccc(-n5c(C([2H])([2H])[2H])nc6ccccc65)cc4)c4ccccc34)c([2H])c([2H])c2c1[2H]]

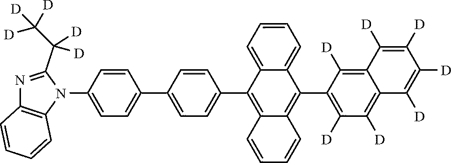 [SMILES: [2H]c1c([2H])c([2H])c2c([2H])c(-c3c4ccccc4c(-c4ccc(-c5ccc(-n6c(C([2H])([2H])C([2H])([2H])[2H])nc7ccccc76)cc5)cc4)c4ccccc34)c([2H])c([2H])c2c1[2H]]

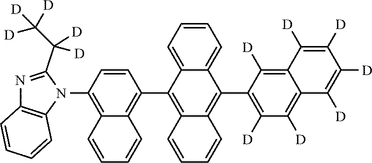 [SMILES: [2H]c1c([2H])c([2H])c2c([2H])c(-c3c4ccccc4c(-c4ccc(-n5c(C([2H])([2H])C([2H])([2H])[2H])nc6ccccc65)c5ccccc45)c4ccccc34)c([2H])c([2H])c2c1[2H]]

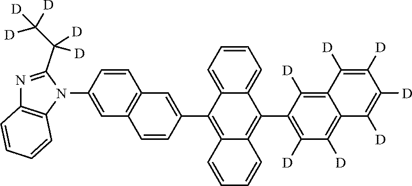 [SMILES: [2H]c1c([2H])c([2H])c2c([2H])c(-c3c4ccccc4c(-c4ccc5cc(-n6c(C([2H])([2H])C([2H])([2H])[2H])nc7ccccc76)ccc5c4)c4ccccc34)c([2H])c([2H])c2c1[2H]]

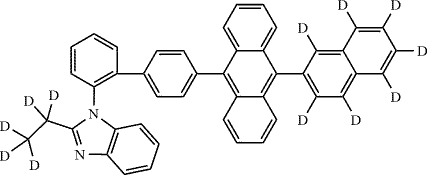 [SMILES: [2H]c1c([2H])c([2H])c2c([2H])c(-c3c4ccccc4c(-c4ccc(-c5ccccc5-n5c(C([2H])([2H])C([2H])([2H])[2H])nc6ccccc65)cc4)c4ccccc34)c([2H])c([2H])c2c1[2H]]

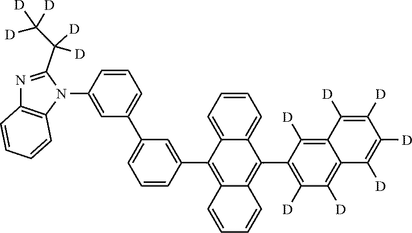 [SMILES: [2H]c1c([2H])c([2H])c2c([2H])c(-c3c4ccccc4c(-c4cccc(-c5cccc(-n6c(C([2H])([2H])C([2H])([2H])[2H])nc7ccccc76)c5)c4)c4ccccc34)c([2H])c([2H])c2c1[2H]]

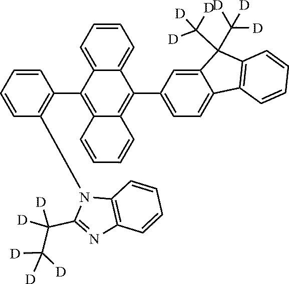 [SMILES: [2H]C([2H])([2H])C([2H])([2H])c1nc2ccccc2n1-c1ccccc1-c1c2ccccc2c(-c2ccc3c(c2)C(C([2H])([2H])[2H])(C([2H])([2H])[2H])c2ccccc2-3)c2ccccc12]

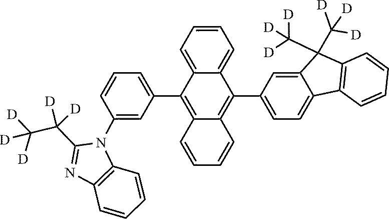 [SMILES: [2H]C([2H])([2H])C([2H])([2H])c1nc2ccccc2n1-c1cccc(-c2c3ccccc3c(-c3ccc4c(c3)C(C([2H])([2H])[2H])(C([2H])([2H])[2H])c3ccccc3-4)c3ccccc23)c1]

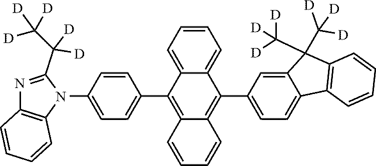 [SMILES: [2H]C([2H])([2H])C([2H])([2H])c1nc2ccccc2n1-c1ccc(-c2c3ccccc3c(-c3ccc4c(c3)C(C([2H])([2H])[2H])(C([2H])([2H])[2H])c3ccccc3-4)c3ccccc23)cc1]

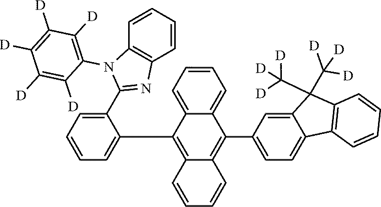 [SMILES: [2H]c1c([2H])c([2H])c(-n2c(-c3ccccc3-c3c4ccccc4c(-c4ccc5c(c4)C(C([2H])([2H])[2H])(C([2H])([2H])[2H])c4ccccc4-5)c4ccccc34)nc3ccccc32)c([2H])c1[2H]]

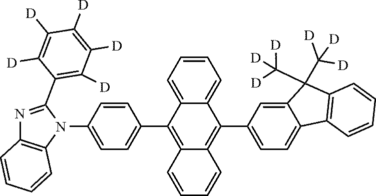 [SMILES: [2H]c1c([2H])c([2H])c(-c2nc3ccccc3n2-c2ccc(-c3c4ccccc4c(-c4ccc5c(c4)C(C([2H])([2H])[2H])(C([2H])([2H])[2H])c4ccccc4-5)c4ccccc34)cc2)c([2H])c1[2H]]

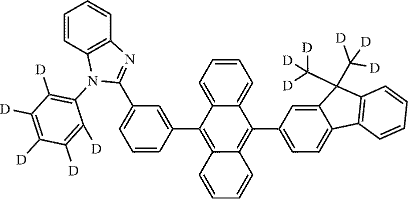 [SMILES: [2H]c1c([2H])c([2H])c(-n2c(-c3cccc(-c4c5ccccc5c(-c5ccc6c(c5)C(C([2H])([2H])[2H])(C([2H])([2H])[2H])c5ccccc5-6)c5ccccc45)c3)nc3ccccc32)c([2H])c1[2H]]

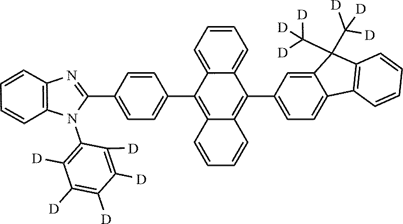 [SMILES: [2H]c1c([2H])c([2H])c(-n2c(-c3ccc(-c4c5ccccc5c(-c5ccc6c(c5)C(C([2H])([2H])[2H])(C([2H])([2H])[2H])c5ccccc5-6)c5ccccc45)cc3)nc3ccccc32)c([2H])c1[2H]]

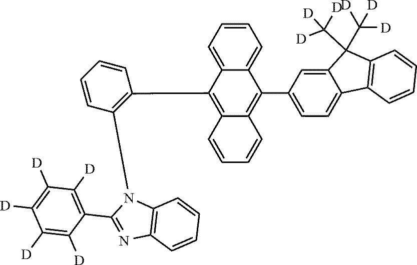 [SMILES: [2H]c1c([2H])c([2H])c(-c2nc3ccccc3n2-c2ccccc2-c2c3ccccc3c(-c3ccc4c(c3)C(C([2H])([2H])[2H])(C([2H])([2H])[2H])c3ccccc3-4)c3ccccc23)c([2H])c1[2H]]

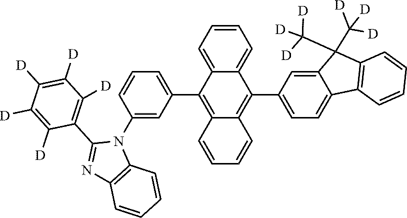 [SMILES: [2H]c1c([2H])c([2H])c(-c2nc3ccccc3n2-c2cccc(-c3c4ccccc4c(-c4ccc5c(c4)C(C([2H])([2H])[2H])(C([2H])([2H])[2H])c4ccccc4-5)c4ccccc34)c2)c([2H])c1[2H]]

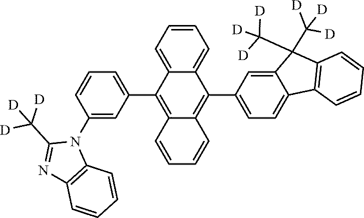 [SMILES: [2H]C([2H])([2H])c1nc2ccccc2n1-c1cccc(-c2c3ccccc3c(-c3ccc4c(c3)C(C([2H])([2H])[2H])(C([2H])([2H])[2H])c3ccccc3-4)c3ccccc23)c1]

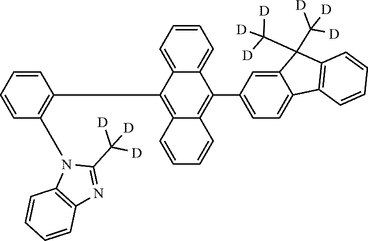 [SMILES: [2H]C([2H])([2H])c1nc2ccccc2n1-c1ccccc1-c1c2ccccc2c(-c2ccc3c(c2)C(C([2H])([2H])[2H])(C([2H])([2H])[2H])c2ccccc2-3)c2ccccc12]

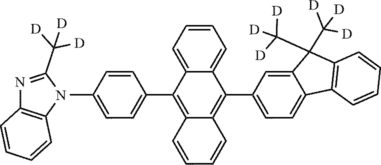 [SMILES: [2H]C([2H])([2H])c1nc2ccccc2n1-c1ccc(-c2c3ccccc3c(-c3ccc4c(c3)C(C([2H])([2H])[2H])(C([2H])([2H])[2H])c3ccccc3-4)c3ccccc23)cc1]

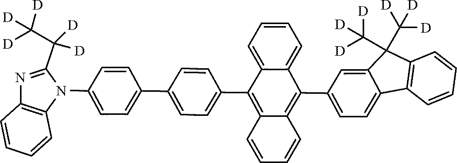 [SMILES: [2H]C([2H])([2H])C([2H])([2H])c1nc2ccccc2n1-c1ccc(-c2ccc(-c3c4ccccc4c(-c4ccc5c(c4)C(C([2H])([2H])[2H])(C([2H])([2H])[2H])c4ccccc4-5)c4ccccc34)cc2)cc1]

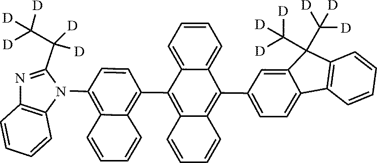 [SMILES: [2H]C([2H])([2H])C([2H])([2H])c1nc2ccccc2n1-c1ccc(-c2c3ccccc3c(-c3ccc4c(c3)C(C([2H])([2H])[2H])(C([2H])([2H])[2H])c3ccccc3-4)c3ccccc23)c2ccccc12]

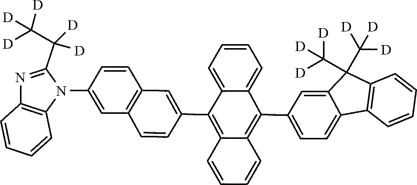 [SMILES: [2H]C([2H])([2H])C([2H])([2H])c1nc2ccccc2n1-c1ccc2cc(-c3c4ccccc4c(-c4ccc5c(c4)C(C([2H])([2H])[2H])(C([2H])([2H])[2H])c4ccccc4-5)c4ccccc34)ccc2c1]

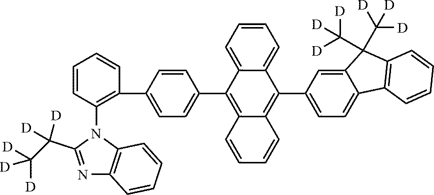 [SMILES: [2H]C([2H])([2H])C([2H])([2H])c1nc2ccccc2n1-c1ccccc1-c1ccc(-c2c3ccccc3c(-c3ccc4c(c3)C(C([2H])([2H])[2H])(C([2H])([2H])[2H])c3ccccc3-4)c3ccccc23)cc1]

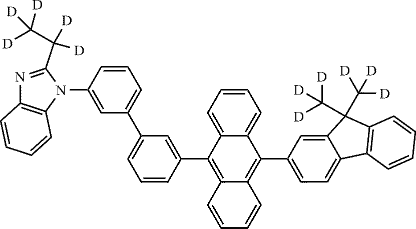 [SMILES: [2H]C([2H])([2H])C([2H])([2H])c1nc2ccccc2n1-c1cccc(-c2cccc(-c3c4ccccc4c(-c4ccc5c(c4)C(C([2H])([2H])[2H])(C([2H])([2H])[2H])c4ccccc4-5)c4ccccc34)c2)c1]

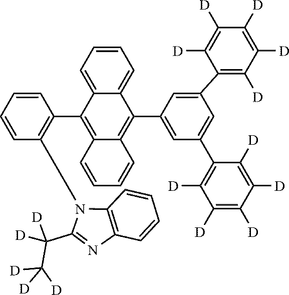 [SMILES: [2H]c1c([2H])c([2H])c(-c2cc(-c3c([2H])c([2H])c([2H])c([2H])c3[2H])cc(-c3c4ccccc4c(-c4ccccc4-n4c(C([2H])([2H])C([2H])([2H])[2H])nc5ccccc54)c4ccccc34)c2)c([2H])c1[2H]]

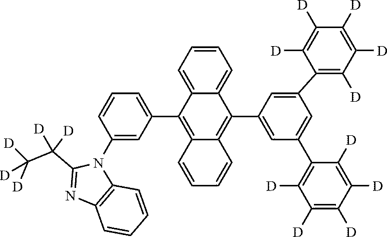 [SMILES: [2H]c1c([2H])c([2H])c(-c2cc(-c3c([2H])c([2H])c([2H])c([2H])c3[2H])cc(-c3c4ccccc4c(-c4cccc(-n5c(C([2H])([2H])C([2H])([2H])[2H])nc6ccccc65)c4)c4ccccc34)c2)c([2H])c1[2H]]

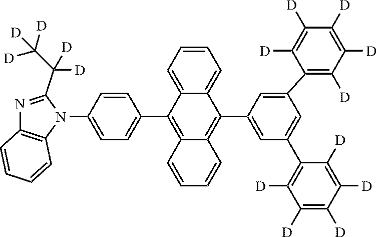 [SMILES: [2H]c1c([2H])c([2H])c(-c2cc(-c3c([2H])c([2H])c([2H])c([2H])c3[2H])cc(-c3c4ccccc4c(-c4ccc(-n5c(C([2H])([2H])C([2H])([2H])[2H])nc6ccccc65)cc4)c4ccccc34)c2)c([2H])c1[2H]]

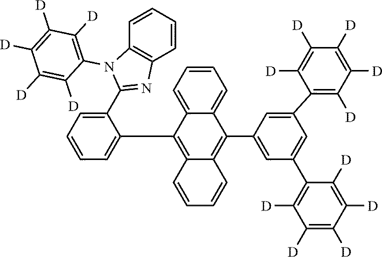 [SMILES: [2H]c1c([2H])c([2H])c(-c2cc(-c3c([2H])c([2H])c([2H])c([2H])c3[2H])cc(-c3c4ccccc4c(-c4ccccc4-c4nc5ccccc5n4-c4c([2H])c([2H])c([2H])c([2H])c4[2H])c4ccccc34)c2)c([2H])c1[2H]]

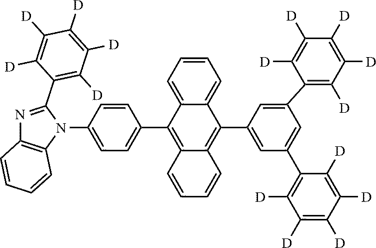 [SMILES: [2H]c1c([2H])c([2H])c(-c2cc(-c3c([2H])c([2H])c([2H])c([2H])c3[2H])cc(-c3c4ccccc4c(-c4ccc(-n5c(-c6c([2H])c([2H])c([2H])c([2H])c6[2H])nc6ccccc65)cc4)c4ccccc34)c2)c([2H])c1[2H]]

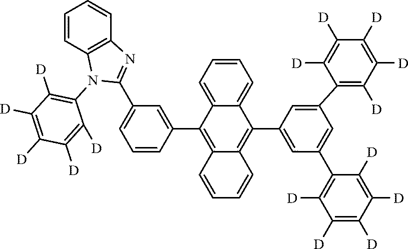 [SMILES: [2H]c1c([2H])c([2H])c(-c2cc(-c3c([2H])c([2H])c([2H])c([2H])c3[2H])cc(-c3c4ccccc4c(-c4cccc(-c5nc6ccccc6n5-c5c([2H])c([2H])c([2H])c([2H])c5[2H])c4)c4ccccc34)c2)c([2H])c1[2H]]

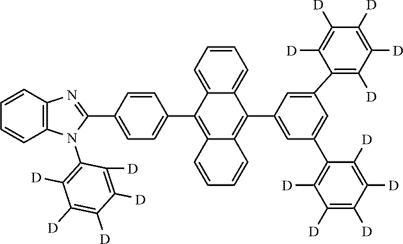 [SMILES: [2H]c1c([2H])c([2H])c(-c2cc(-c3c([2H])c([2H])c([2H])c([2H])c3[2H])cc(-c3c4ccccc4c(-c4ccc(-c5nc6ccccc6n5-c5c([2H])c([2H])c([2H])c([2H])c5[2H])cc4)c4ccccc34)c2)c([2H])c1[2H]]

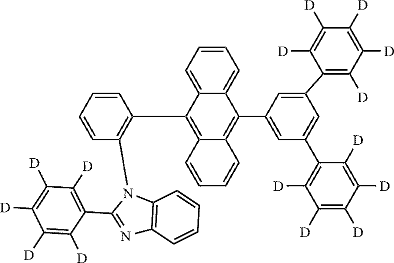 [SMILES: [2H]c1c([2H])c([2H])c(-c2cc(-c3c([2H])c([2H])c([2H])c([2H])c3[2H])cc(-c3c4ccccc4c(-c4ccccc4-n4c(-c5c([2H])c([2H])c([2H])c([2H])c5[2H])nc5ccccc54)c4ccccc34)c2)c([2H])c1[2H]]

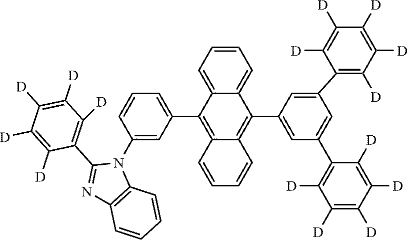 [SMILES: [2H]c1c([2H])c([2H])c(-c2cc(-c3c([2H])c([2H])c([2H])c([2H])c3[2H])cc(-c3c4ccccc4c(-c4cccc(-n5c(-c6c([2H])c([2H])c([2H])c([2H])c6[2H])nc6ccccc65)c4)c4ccccc34)c2)c([2H])c1[2H]]

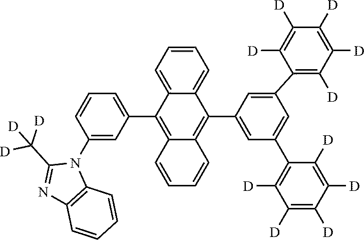 [SMILES: [2H]c1c([2H])c([2H])c(-c2cc(-c3c([2H])c([2H])c([2H])c([2H])c3[2H])cc(-c3c4ccccc4c(-c4cccc(-n5c(C([2H])([2H])[2H])nc6ccccc65)c4)c4ccccc34)c2)c([2H])c1[2H]]

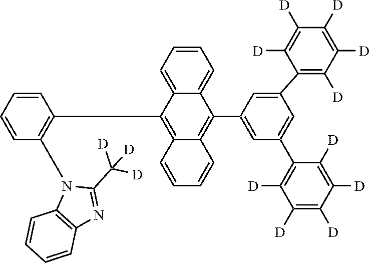 [SMILES: [2H]c1c([2H])c([2H])c(-c2cc(-c3c([2H])c([2H])c([2H])c([2H])c3[2H])cc(-c3c4ccccc4c(-c4ccccc4-n4c(C([2H])([2H])[2H])nc5ccccc54)c4ccccc34)c2)c([2H])c1[2H]]

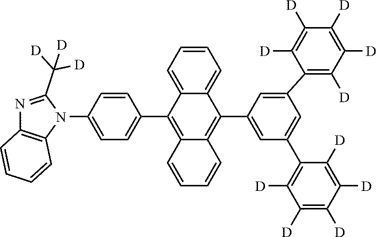 [SMILES: [2H]c1c([2H])c([2H])c(-c2cc(-c3c([2H])c([2H])c([2H])c([2H])c3[2H])cc(-c3c4ccccc4c(-c4ccc(-n5c(C([2H])([2H])[2H])nc6ccccc65)cc4)c4ccccc34)c2)c([2H])c1[2H]]

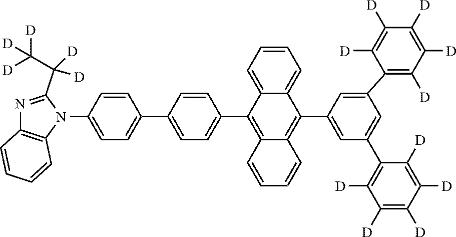 [SMILES: [2H]c1c([2H])c([2H])c(-c2cc(-c3c([2H])c([2H])c([2H])c([2H])c3[2H])cc(-c3c4ccccc4c(-c4ccc(-c5ccc(-n6c(C([2H])([2H])C([2H])([2H])[2H])nc7ccccc76)cc5)cc4)c4ccccc34)c2)c([2H])c1[2H]]

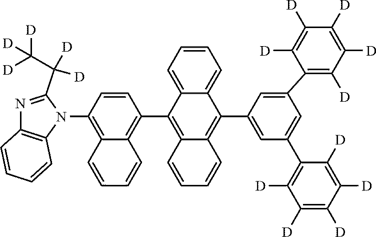 [SMILES: [2H]c1c([2H])c([2H])c(-c2cc(-c3c([2H])c([2H])c([2H])c([2H])c3[2H])cc(-c3c4ccccc4c(-c4ccc(-n5c(C([2H])([2H])C([2H])([2H])[2H])nc6ccccc65)c5ccccc45)c4ccccc34)c2)c([2H])c1[2H]]

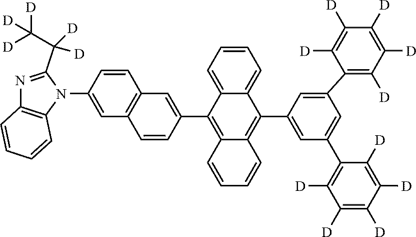 [SMILES: [2H]c1c([2H])c([2H])c(-c2cc(-c3c([2H])c([2H])c([2H])c([2H])c3[2H])cc(-c3c4ccccc4c(-c4ccc5cc(-n6c(C([2H])([2H])C([2H])([2H])[2H])nc7ccccc76)ccc5c4)c4ccccc34)c2)c([2H])c1[2H]]

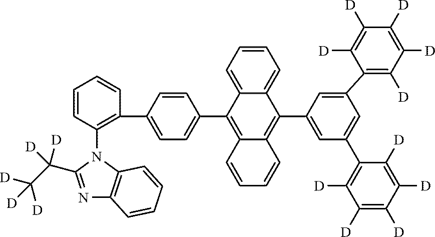 [SMILES: [2H]c1c([2H])c([2H])c(-c2cc(-c3c([2H])c([2H])c([2H])c([2H])c3[2H])cc(-c3c4ccccc4c(-c4ccc(-c5ccccc5-n5c(C([2H])([2H])C([2H])([2H])[2H])nc6ccccc65)cc4)c4ccccc34)c2)c([2H])c1[2H]]

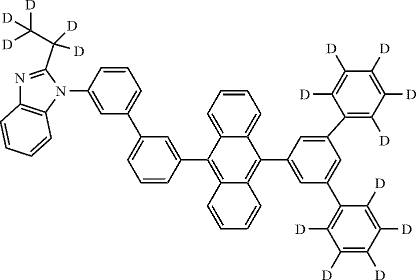 [SMILES: [2H]c1c([2H])c([2H])c(-c2cc(-c3c([2H])c([2H])c([2H])c([2H])c3[2H])cc(-c3c4ccccc4c(-c4cccc(-c5cccc(-n6c(C([2H])([2H])C([2H])([2H])[2H])nc7ccccc76)c5)c4)c4ccccc34)c2)c([2H])c1[2H]]

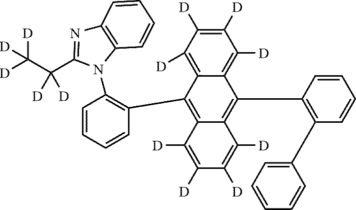 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3ccccc3-c3ccccc3)c2c1[2H]]

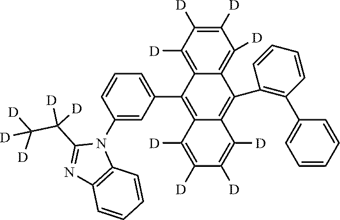 [SMILES: [2H]c1c([2H])c([2H])c2c(-c3ccccc3-c3ccccc3)c3c([2H])c([2H])c([2H])c([2H])c3c(-c3cccc(-n4c(C([2H])([2H])C([2H])([2H])[2H])nc5ccccc54)c3)c2c1[2H]]

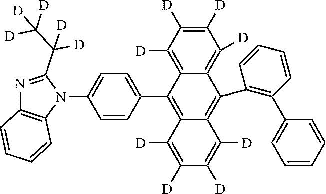 [SMILES: [2H]c1c([2H])c([2H])c2c(-c3ccccc3-c3ccccc3)c3c([2H])c([2H])c([2H])c([2H])c3c(-c3ccc(-n4c(C([2H])([2H])C([2H])([2H])[2H])nc5ccccc54)cc3)c2c1[2H]]

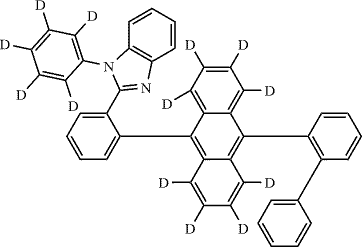 [SMILES: [2H]c1c([2H])c([2H])c(-n2c(-c3ccccc3-c3c4c([2H])c([2H])c([2H])c([2H])c4c(-c4ccccc4-c4ccccc4)c4c([2H])c([2H])c([2H])c([2H])c34)nc3ccccc32)c([2H])c1[2H]]

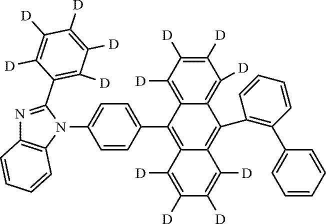 [SMILES: [2H]c1c([2H])c([2H])c(-c2nc3ccccc3n2-c2ccc(-c3c4c([2H])c([2H])c([2H])c([2H])c4c(-c4ccccc4-c4ccccc4)c4c([2H])c([2H])c([2H])c([2H])c34)cc2)c([2H])c1[2H]]

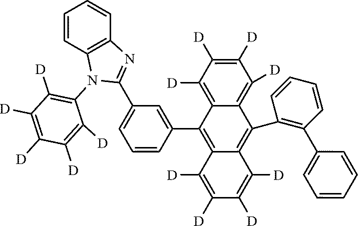 [SMILES: [2H]c1c([2H])c([2H])c(-n2c(-c3cccc(-c4c5c([2H])c([2H])c([2H])c([2H])c5c(-c5ccccc5-c5ccccc5)c5c([2H])c([2H])c([2H])c([2H])c45)c3)nc3ccccc32)c([2H])c1[2H]]

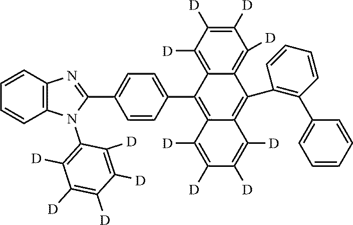 [SMILES: [2H]c1c([2H])c([2H])c(-n2c(-c3ccc(-c4c5c([2H])c([2H])c([2H])c([2H])c5c(-c5ccccc5-c5ccccc5)c5c([2H])c([2H])c([2H])c([2H])c45)cc3)nc3ccccc32)c([2H])c1[2H]]

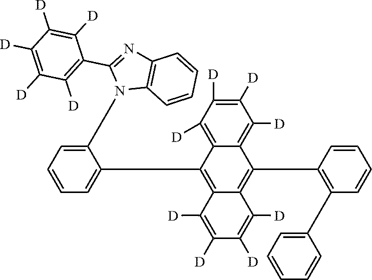 [SMILES: [2H]c1c([2H])c([2H])c(-c2nc3ccccc3n2-c2ccccc2-c2c3c([2H])c([2H])c([2H])c([2H])c3c(-c3ccccc3-c3ccccc3)c3c([2H])c([2H])c([2H])c([2H])c23)c([2H])c1[2H]]

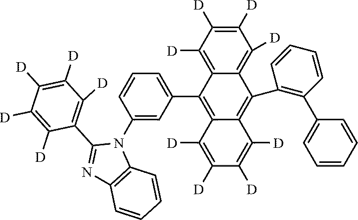 [SMILES: [2H]c1c([2H])c([2H])c(-c2nc3ccccc3n2-c2cccc(-c3c4c([2H])c([2H])c([2H])c([2H])c4c(-c4ccccc4-c4ccccc4)c4c([2H])c([2H])c([2H])c([2H])c34)c2)c([2H])c1[2H]]

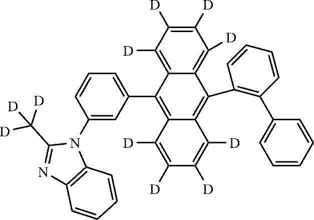 [SMILES: [2H]c1c([2H])c([2H])c2c(-c3ccccc3-c3ccccc3)c3c([2H])c([2H])c([2H])c([2H])c3c(-c3cccc(-n4c(C([2H])([2H])[2H])nc5ccccc54)c3)c2c1[2H]]

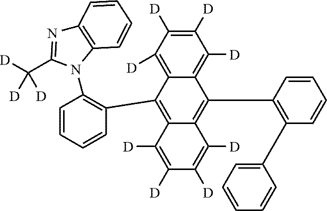 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3ccccc3-c3ccccc3)c2c1[2H]]

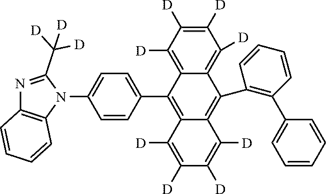 [SMILES: [2H]c1c([2H])c([2H])c2c(-c3ccccc3-c3ccccc3)c3c([2H])c([2H])c([2H])c([2H])c3c(-c3ccc(-n4c(C([2H])([2H])[2H])nc5ccccc54)cc3)c2c1[2H]]

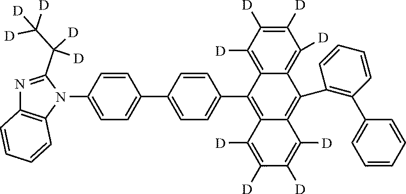 [SMILES: [2H]c1c([2H])c([2H])c2c(-c3ccccc3-c3ccccc3)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

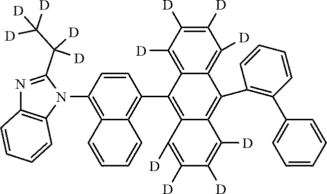 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccccc3-c3ccccc3)c2c1[2H]]

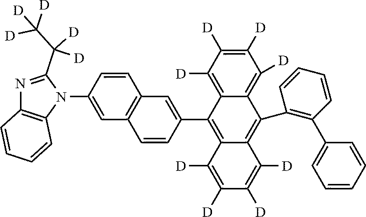 [SMILES: [2H]c1c([2H])c([2H])c2c(-c3ccccc3-c3ccccc3)c3c([2H])c([2H])c([2H])c([2H])c3c(-c3ccc4cc(-n5c(C([2H])([2H])C([2H])([2H])[2H])nc6ccccc65)ccc4c3)c2c1[2H]]

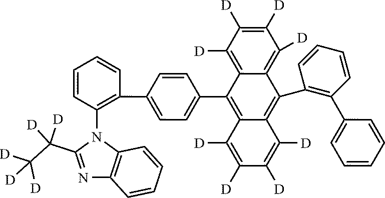 [SMILES: [2H]c1c([2H])c([2H])c2c(-c3ccccc3-c3ccccc3)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

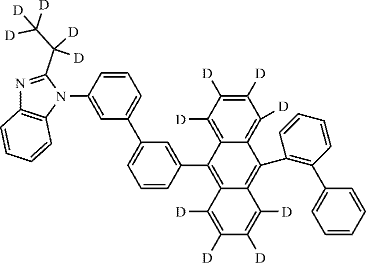 [SMILES: [2H]c1c([2H])c([2H])c2c(-c3ccccc3-c3ccccc3)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

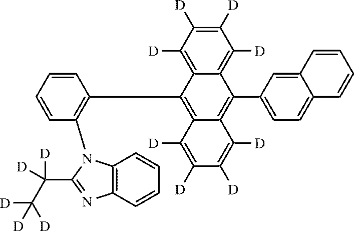 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3ccc4ccccc4c3)c2c1[2H]]

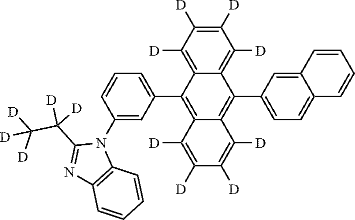 [SMILES: [2H]c1c([2H])c([2H])c2c(-c3ccc4ccccc4c3)c3c([2H])c([2H])c([2H])c([2H])c3c(-c3cccc(-n4c(C([2H])([2H])C([2H])([2H])[2H])nc5ccccc54)c3)c2c1[2H]]

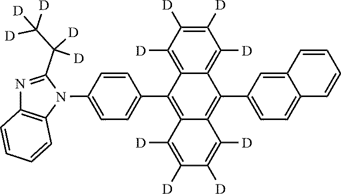 [SMILES: [2H]c1c([2H])c([2H])c2c(-c3ccc4ccccc4c3)c3c([2H])c([2H])c([2H])c([2H])c3c(-c3ccc(-n4c(C([2H])([2H])C([2H])([2H])[2H])nc5ccccc54)cc3)c2c1[2H]]

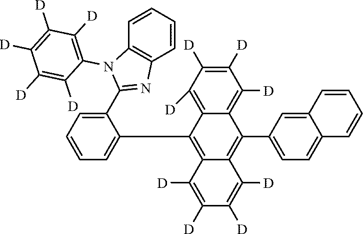 [SMILES: [2H]c1c([2H])c([2H])c(-n2c(-c3ccccc3-c3c4c([2H])c([2H])c([2H])c([2H])c4c(-c4ccc5ccccc5c4)c4c([2H])c([2H])c([2H])c([2H])c34)nc3ccccc32)c([2H])c1[2H]]

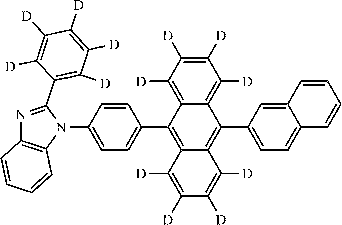 [SMILES: [2H]c1c([2H])c([2H])c(-c2nc3ccccc3n2-c2ccc(-c3c4c([2H])c([2H])c([2H])c([2H])c4c(-c4ccc5ccccc5c4)c4c([2H])c([2H])c([2H])c([2H])c34)cc2)c([2H])c1[2H]]

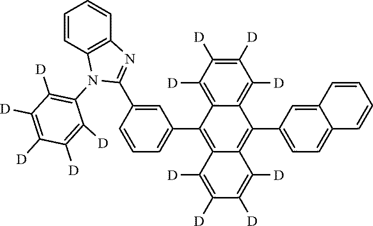 [SMILES: [2H]c1c([2H])c([2H])c(-n2c(-c3cccc(-c4c5c([2H])c([2H])c([2H])c([2H])c5c(-c5ccc6ccccc6c5)c5c([2H])c([2H])c([2H])c([2H])c45)c3)nc3ccccc32)c([2H])c1[2H]]

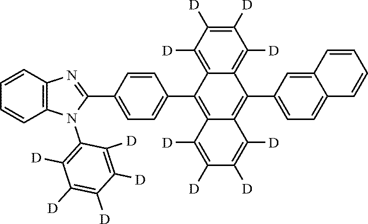 [SMILES: [2H]c1c([2H])c([2H])c(-n2c(-c3ccc(-c4c5c([2H])c([2H])c([2H])c([2H])c5c(-c5ccc6ccccc6c5)c5c([2H])c([2H])c([2H])c([2H])c45)cc3)nc3ccccc32)c([2H])c1[2H]]

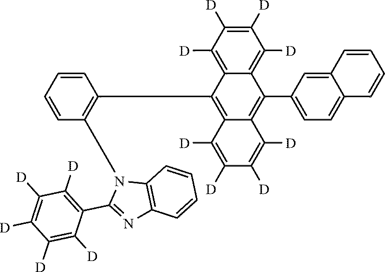 [SMILES: [2H]c1c([2H])c([2H])c(-c2nc3ccccc3n2-c2ccccc2-c2c3c([2H])c([2H])c([2H])c([2H])c3c(-c3ccc4ccccc4c3)c3c([2H])c([2H])c([2H])c([2H])c23)c([2H])c1[2H]]

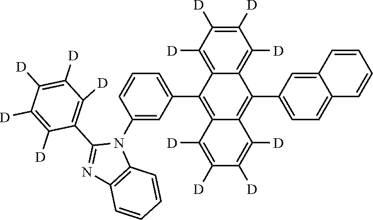 [SMILES: [2H]c1c([2H])c([2H])c(-c2nc3ccccc3n2-c2cccc(-c3c4c([2H])c([2H])c([2H])c([2H])c4c(-c4ccc5ccccc5c4)c4c([2H])c([2H])c([2H])c([2H])c34)c2)c([2H])c1[2H]]

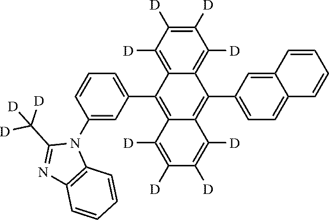 [SMILES: [2H]c1c([2H])c([2H])c2c(-c3ccc4ccccc4c3)c3c([2H])c([2H])c([2H])c([2H])c3c(-c3cccc(-n4c(C([2H])([2H])[2H])nc5ccccc54)c3)c2c1[2H]]

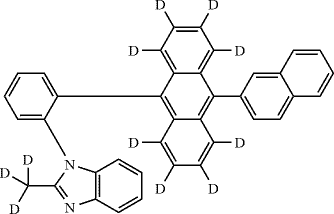 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3ccc4ccccc4c3)c2c1[2H]]

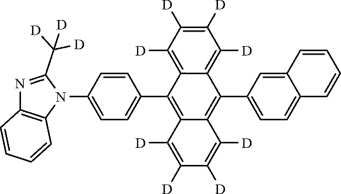 [SMILES: [2H]c1c([2H])c([2H])c2c(-c3ccc4ccccc4c3)c3c([2H])c([2H])c([2H])c([2H])c3c(-c3ccc(-n4c(C([2H])([2H])[2H])nc5ccccc54)cc3)c2c1[2H]]

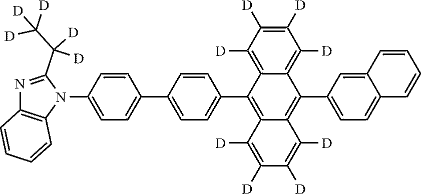 [SMILES: [2H]c1c([2H])c([2H])c2c(-c3ccc4ccccc4c3)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

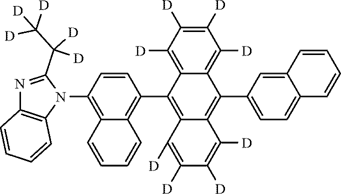 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccc4ccccc4c3)c2c1[2H]]

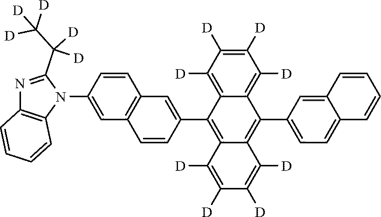 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3ccc4ccccc4c3)c2c1[2H]]

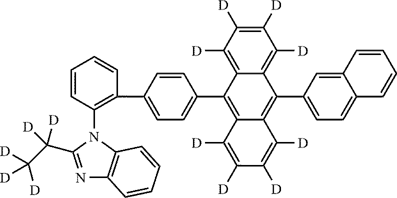 [SMILES: [2H]c1c([2H])c([2H])c2c(-c3ccc4ccccc4c3)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

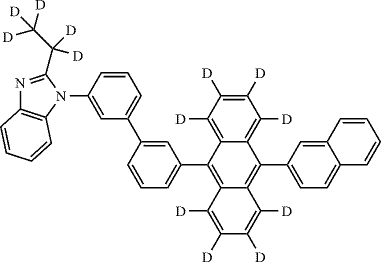 [SMILES: [2H]c1c([2H])c([2H])c2c(-c3ccc4ccccc4c3)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

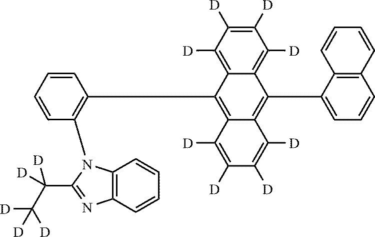 [SMILES: [2H]c1c([2H])c([2H])c2c(-c3cccc4ccccc34)c3c([2H])c([2H])c([2H])c([2H])c3c(-c3ccccc3-n3c(C([2H])([2H])C([2H])([2H])[2H])nc4ccccc43)c2c1[2H]]

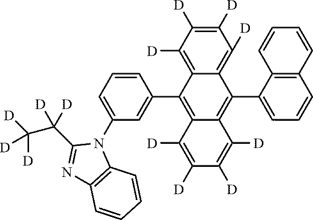 [SMILES: [2H]c1c([2H])c([2H])c2c(-c3cccc4ccccc34)c3c([2H])c([2H])c([2H])c([2H])c3c(-c3cccc(-n4c(C([2H])([2H])C([2H])([2H])[2H])nc5ccccc54)c3)c2c1[2H]]

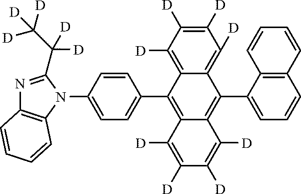 [SMILES: [2H]c1c([2H])c([2H])c2c(-c3cccc4ccccc34)c3c([2H])c([2H])c([2H])c([2H])c3c(-c3ccc(-n4c(C([2H])([2H])C([2H])([2H])[2H])nc5ccccc54)cc3)c2c1[2H]]

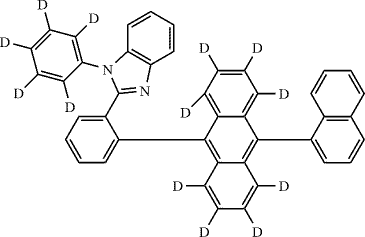 [SMILES: [2H]c1c([2H])c([2H])c(-n2c(-c3ccccc3-c3c4c([2H])c([2H])c([2H])c([2H])c4c(-c4cccc5ccccc45)c4c([2H])c([2H])c([2H])c([2H])c34)nc3ccccc32)c([2H])c1[2H]]

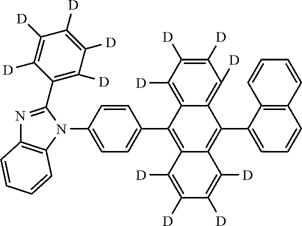 [SMILES: [2H]c1c([2H])c([2H])c(-c2nc3ccccc3n2-c2ccc(-c3c4c([2H])c([2H])c([2H])c([2H])c4c(-c4cccc5ccccc45)c4c([2H])c([2H])c([2H])c([2H])c34)cc2)c([2H])c1[2H]]

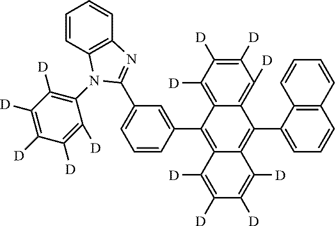 [SMILES: [2H]c1c([2H])c([2H])c(-n2c(-c3cccc(-c4c5c([2H])c([2H])c([2H])c([2H])c5c(-c5cccc6ccccc56)c5c([2H])c([2H])c([2H])c([2H])c45)c3)nc3ccccc32)c([2H])c1[2H]]

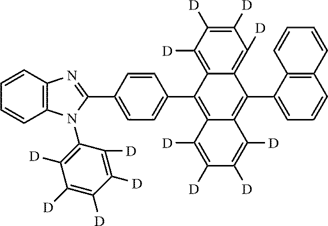 [SMILES: [2H]c1c([2H])c([2H])c(-n2c(-c3ccc(-c4c5c([2H])c([2H])c([2H])c([2H])c5c(-c5cccc6ccccc56)c5c([2H])c([2H])c([2H])c([2H])c45)cc3)nc3ccccc32)c([2H])c1[2H]]

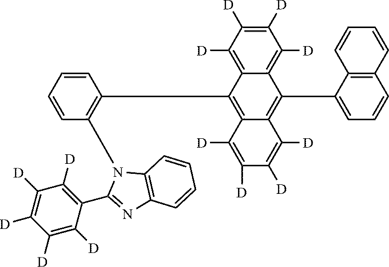 [SMILES: [2H]c1c([2H])c([2H])c(-c2nc3ccccc3n2-c2ccccc2-c2c3c([2H])c([2H])c([2H])c([2H])c3c(-c3cccc4ccccc34)c3c([2H])c([2H])c([2H])c([2H])c23)c([2H])c1[2H]]

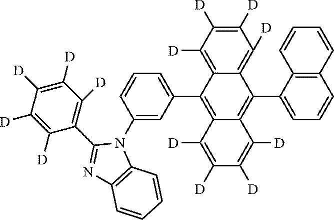 [SMILES: [2H]c1c([2H])c([2H])c(-c2nc3ccccc3n2-c2cccc(-c3c4c([2H])c([2H])c([2H])c([2H])c4c(-c4cccc5ccccc45)c4c([2H])c([2H])c([2H])c([2H])c34)c2)c([2H])c1[2H]]

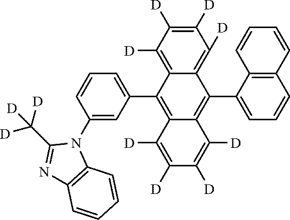 [SMILES: [2H]c1c([2H])c([2H])c2c(-c3cccc4ccccc34)c3c([2H])c([2H])c([2H])c([2H])c3c(-c3cccc(-n4c(C([2H])([2H])[2H])nc5ccccc54)c3)c2c1[2H]]

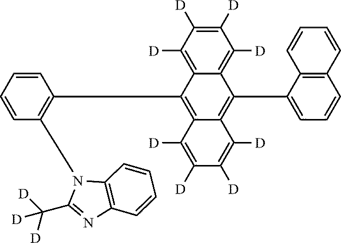 [SMILES: [2H]c1c([2H])c([2H])c2c(-c3cccc4ccccc34)c3c([2H])c([2H])c([2H])c([2H])c3c(-c3ccccc3-n3c(C([2H])([2H])[2H])nc4ccccc43)c2c1[2H]]

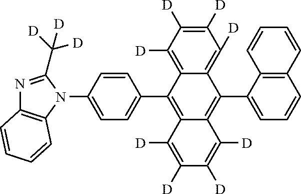 [SMILES: [2H]c1c([2H])c([2H])c2c(-c3cccc4ccccc34)c3c([2H])c([2H])c([2H])c([2H])c3c(-c3ccc(-n4c(C([2H])([2H])[2H])nc5ccccc54)cc3)c2c1[2H]]

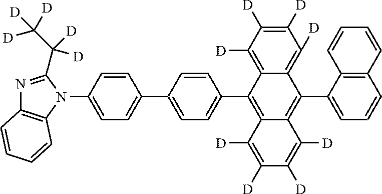 [SMILES: [2H]c1c([2H])c([2H])c2c(-c3cccc4ccccc34)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

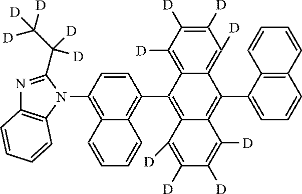 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3cccc4ccccc34)c2c1[2H]]

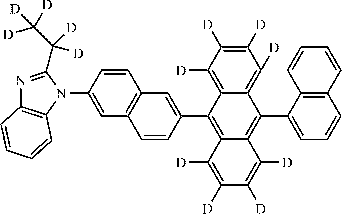 [SMILES: [2H]c1c([2H])c([2H])c2c(-c3cccc4ccccc34)c3c([2H])c([2H])c([2H])c([2H])c3c(-c3ccc4cc(-n5c(C([2H])([2H])C([2H])([2H])[2H])nc6ccccc65)ccc4c3)c2c1[2H]]

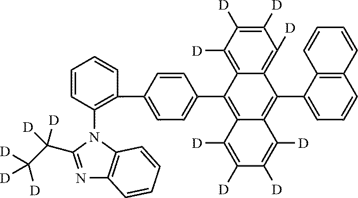 [SMILES: [2H]c1c([2H])c([2H])c2c(-c3cccc4ccccc34)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

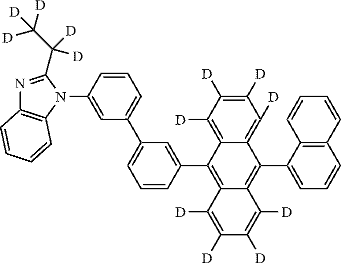 [SMILES: [2H]c1c([2H])c([2H])c2c(-c3cccc4ccccc34)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

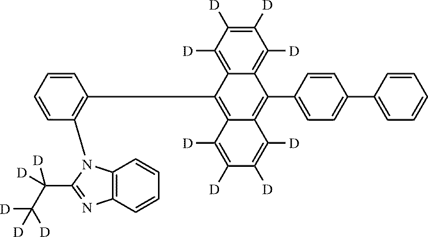 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3ccc(-c4ccccc4)cc3)c2c1[2H]]

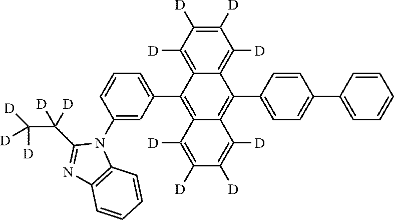 [SMILES: [2H]c1c([2H])c([2H])c2c(-c3cccc(-n4c(C([2H])([2H])C([2H])([2H])[2H])nc5ccccc54)c3)c3c([2H])c([2H])c([2H])c([2H])c3c(-c3ccc(-c4ccccc4)cc3)c2c1[2H]]

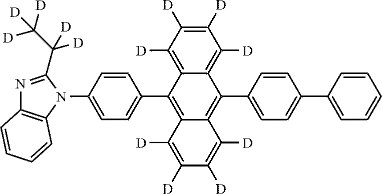 [SMILES: [2H]c1c([2H])c([2H])c2c(-c3ccc(-n4c(C([2H])([2H])C([2H])([2H])[2H])nc5ccccc54)cc3)c3c([2H])c([2H])c([2H])c([2H])c3c(-c3ccc(-c4ccccc4)cc3)c2c1[2H]]

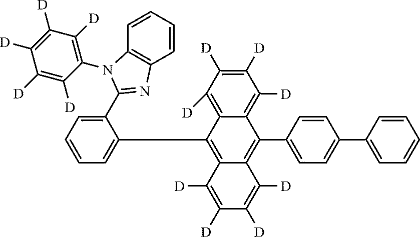 [SMILES: [2H]c1c([2H])c([2H])c(-n2c(-c3ccccc3-c3c4c([2H])c([2H])c([2H])c([2H])c4c(-c4ccc(-c5ccccc5)cc4)c4c([2H])c([2H])c([2H])c([2H])c34)nc3ccccc32)c([2H])c1[2H]]

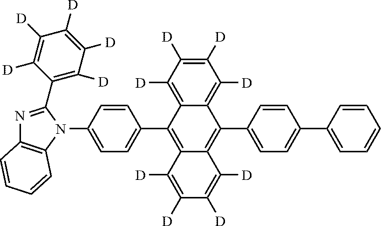 [SMILES: [2H]c1c([2H])c([2H])c(-c2nc3ccccc3n2-c2ccc(-c3c4c([2H])c([2H])c([2H])c([2H])c4c(-c4ccc(-c5ccccc5)cc4)c4c([2H])c([2H])c([2H])c([2H])c34)cc2)c([2H])c1[2H]]

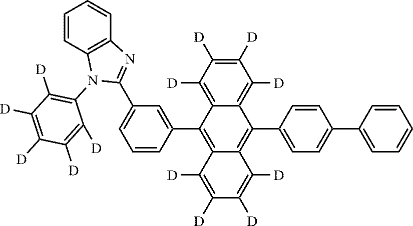 [SMILES: [2H]c1c([2H])c([2H])c(-n2c(-c3cccc(-c4c5c([2H])c([2H])c([2H])c([2H])c5c(-c5ccc(-c6ccccc6)cc5)c5c([2H])c([2H])c([2H])c([2H])c45)c3)nc3ccccc32)c([2H])c1[2H]]

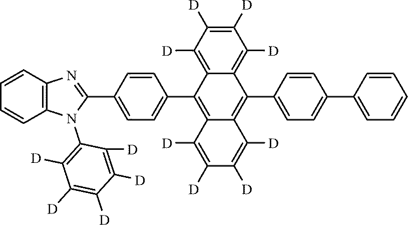 [SMILES: [2H]c1c([2H])c([2H])c(-n2c(-c3ccc(-c4c5c([2H])c([2H])c([2H])c([2H])c5c(-c5ccc(-c6ccccc6)cc5)c5c([2H])c([2H])c([2H])c([2H])c45)cc3)nc3ccccc32)c([2H])c1[2H]]

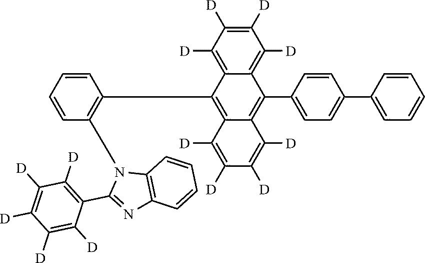 [SMILES: [2H]c1c([2H])c([2H])c(-c2nc3ccccc3n2-c2ccccc2-c2c3c([2H])c([2H])c([2H])c([2H])c3c(-c3ccc(-c4ccccc4)cc3)c3c([2H])c([2H])c([2H])c([2H])c23)c([2H])c1[2H]]

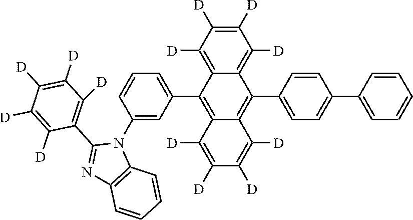 [SMILES: [2H]c1c([2H])c([2H])c(-c2nc3ccccc3n2-c2cccc(-c3c4c([2H])c([2H])c([2H])c([2H])c4c(-c4ccc(-c5ccccc5)cc4)c4c([2H])c([2H])c([2H])c([2H])c34)c2)c([2H])c1[2H]]

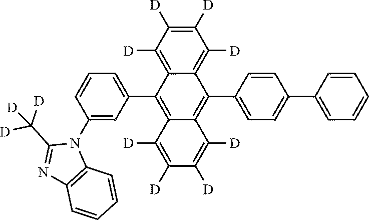 [SMILES: [2H]c1c([2H])c([2H])c2c(-c3cccc(-n4c(C([2H])([2H])[2H])nc5ccccc54)c3)c3c([2H])c([2H])c([2H])c([2H])c3c(-c3ccc(-c4ccccc4)cc3)c2c1[2H]]

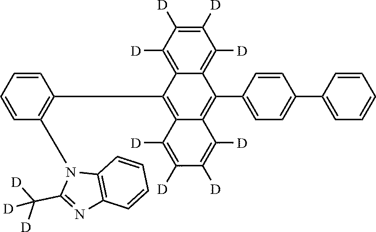 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3ccc(-c4ccccc4)cc3)c2c1[2H]]

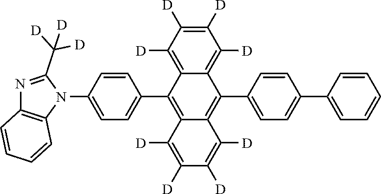 [SMILES: [2H]c1c([2H])c([2H])c2c(-c3ccc(-n4c(C([2H])([2H])[2H])nc5ccccc54)cc3)c3c([2H])c([2H])c([2H])c([2H])c3c(-c3ccc(-c4ccccc4)cc3)c2c1[2H]]

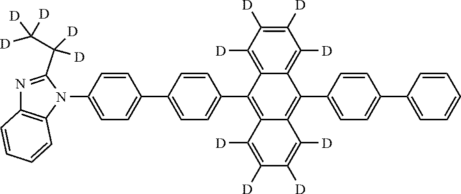 [SMILES: [2H]c1c([2H])c([2H])c2c(-c3ccc(-c4ccc(-n5c(C([2H])([2H])C([2H])([2H])[2H])nc6ccccc65)cc4)cc3)c3c([2H])c([2H])c([2H])c([2H])c3c(-c3ccc(-c4ccccc4)cc3)c2c1[2H]]

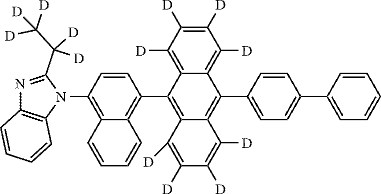 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccc(-c4ccccc4)cc3)c2c1[2H]]

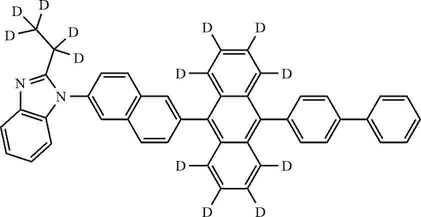 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3ccc(-c4ccccc4)cc3)c2c1[2H]]

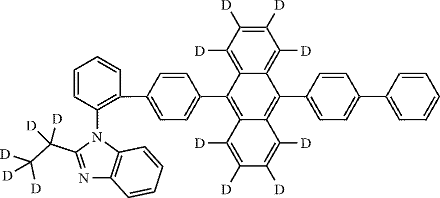 [SMILES: [2H]c1c([2H])c([2H])c2c(-c3ccc(-c4ccccc4-n4c(C([2H])([2H])C([2H])([2H])[2H])nc5ccccc54)cc3)c3c([2H])c([2H])c([2H])c([2H])c3c(-c3ccc(-c4ccccc4)cc3)c2c1[2H]]

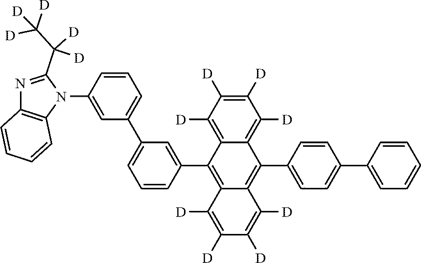 [SMILES: [2H]c1c([2H])c([2H])c2c(-c3cccc(-c4cccc(-n5c(C([2H])([2H])C([2H])([2H])[2H])nc6ccccc65)c4)c3)c3c([2H])c([2H])c([2H])c([2H])c3c(-c3ccc(-c4ccccc4)cc3)c2c1[2H]]

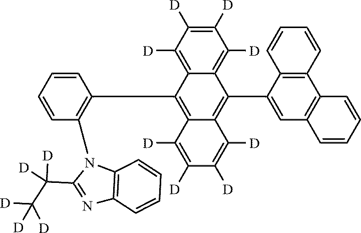 [SMILES: [2H]c1c([2H])c([2H])c2c(-c3cc4ccccc4c4ccccc34)c3c([2H])c([2H])c([2H])c([2H])c3c(-c3ccccc3-n3c(C([2H])([2H])C([2H])([2H])[2H])nc4ccccc43)c2c1[2H]]

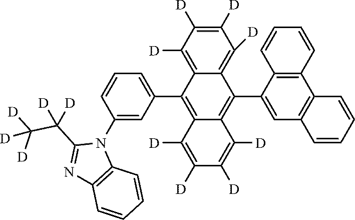 [SMILES: [2H]c1c([2H])c([2H])c2c(-c3cc4ccccc4c4ccccc34)c3c([2H])c([2H])c([2H])c([2H])c3c(-c3cccc(-n4c(C([2H])([2H])C([2H])([2H])[2H])nc5ccccc54)c3)c2c1[2H]]

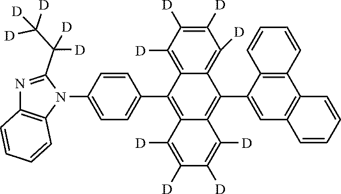 [SMILES: [2H]c1c([2H])c([2H])c2c(-c3cc4ccccc4c4ccccc34)c3c([2H])c([2H])c([2H])c([2H])c3c(-c3ccc(-n4c(C([2H])([2H])C([2H])([2H])[2H])nc5ccccc54)cc3)c2c1[2H]]

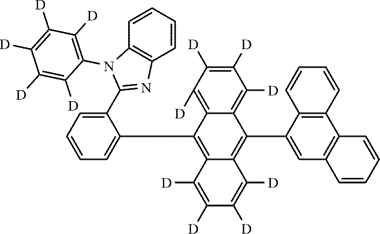 [SMILES: [2H]c1c([2H])c([2H])c(-n2c(-c3ccccc3-c3c4c([2H])c([2H])c([2H])c([2H])c4c(-c4cc5ccccc5c5ccccc45)c4c([2H])c([2H])c([2H])c([2H])c34)nc3ccccc32)c([2H])c1[2H]]

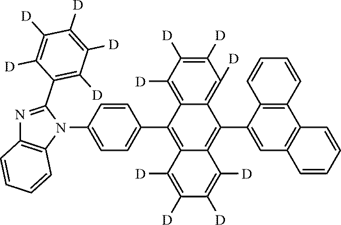 [SMILES: [2H]c1c([2H])c([2H])c(-c2nc3ccccc3n2-c2ccc(-c3c4c([2H])c([2H])c([2H])c([2H])c4c(-c4cc5ccccc5c5ccccc45)c4c([2H])c([2H])c([2H])c([2H])c34)cc2)c([2H])c1[2H]]

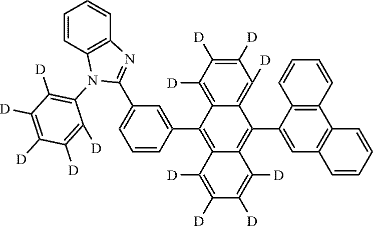 [SMILES: [2H]c1c([2H])c([2H])c(-n2c(-c3cccc(-c4c5c([2H])c([2H])c([2H])c([2H])c5c(-c5cc6ccccc6c6ccccc56)c5c([2H])c([2H])c([2H])c([2H])c45)c3)nc3ccccc32)c([2H])c1[2H]]

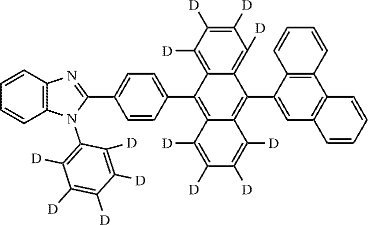 [SMILES: [2H]c1c([2H])c([2H])c(-n2c(-c3ccc(-c4c5c([2H])c([2H])c([2H])c([2H])c5c(-c5cc6ccccc6c6ccccc56)c5c([2H])c([2H])c([2H])c([2H])c45)cc3)nc3ccccc32)c([2H])c1[2H]]

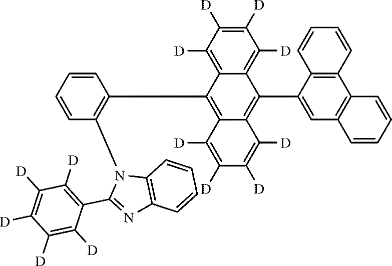 [SMILES: [2H]c1c([2H])c([2H])c(-c2nc3ccccc3n2-c2ccccc2-c2c3c([2H])c([2H])c([2H])c([2H])c3c(-c3cc4ccccc4c4ccccc34)c3c([2H])c([2H])c([2H])c([2H])c23)c([2H])c1[2H]]

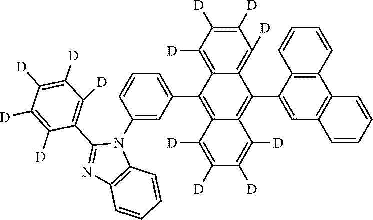 [SMILES: [2H]c1c([2H])c([2H])c(-c2nc3ccccc3n2-c2cccc(-c3c4c([2H])c([2H])c([2H])c([2H])c4c(-c4cc5ccccc5c5ccccc45)c4c([2H])c([2H])c([2H])c([2H])c34)c2)c([2H])c1[2H]]

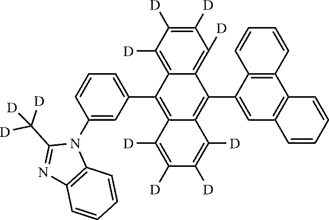 [SMILES: [2H]c1c([2H])c([2H])c2c(-c3cc4ccccc4c4ccccc34)c3c([2H])c([2H])c([2H])c([2H])c3c(-c3cccc(-n4c(C([2H])([2H])[2H])nc5ccccc54)c3)c2c1[2H]]

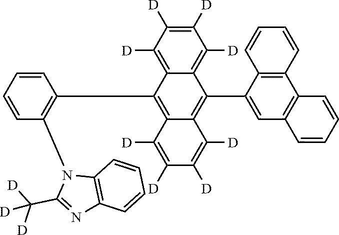 [SMILES: [2H]c1c([2H])c([2H])c2c(-c3cc4ccccc4c4ccccc34)c3c([2H])c([2H])c([2H])c([2H])c3c(-c3ccccc3-n3c(C([2H])([2H])[2H])nc4ccccc43)c2c1[2H]]

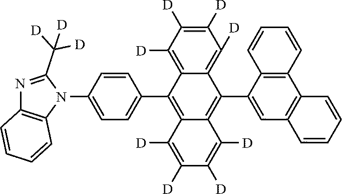 [SMILES: [2H]c1c([2H])c([2H])c2c(-c3cc4ccccc4c4ccccc34)c3c([2H])c([2H])c([2H])c([2H])c3c(-c3ccc(-n4c(C([2H])([2H])[2H])nc5ccccc54)cc3)c2c1[2H]]

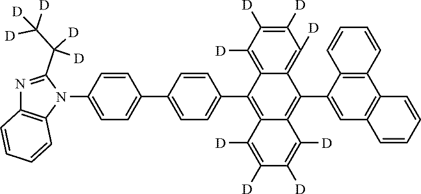 [SMILES: [2H]c1c([2H])c([2H])c2c(-c3cc4ccccc4c4ccccc34)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

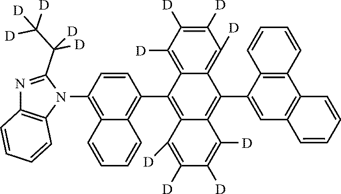 [SMILES: [2H]c1c([2H])c([2H])c2c(-c3cc4ccccc4c4ccccc34)c3c([2H])c([2H])c([2H])c([2H])c3c(-c3ccc(-n4c(C([2H])([2H])C([2H])([2H])[2H])nc5ccccc54)c4ccccc34)c2c1[2H]]

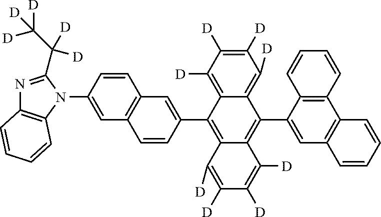 [SMILES: [2H]c1c([2H])c([2H])c2c(-c3cc4ccccc4c4ccccc34)c3c([2H])c([2H])c([2H])c([2H])c3c(-c3ccc4cc(-n5c(C([2H])([2H])C([2H])([2H])[2H])nc6ccccc65)ccc4c3)c2c1[2H]]

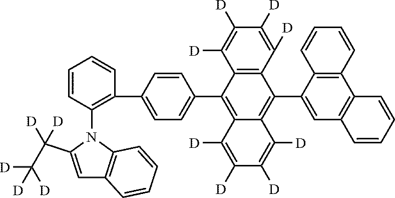 [SMILES: [2H]c1c([2H])c([2H])c2c(-c3cc4ccccc4c4ccccc34)c3c([2H])c([2H])c([2H])c([2H])c3c(-c3ccc(-c4ccccc4-n4c(C([2H])([2H])C([2H])([2H])[2H])cc5ccccc54)cc3)c2c1[2H]]

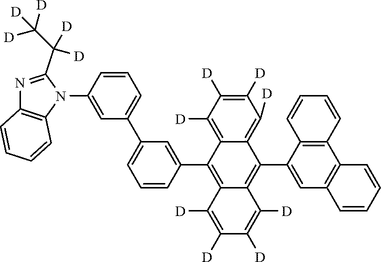 [SMILES: [2H]c1c([2H])c([2H])c2c(-c3cc4ccccc4c4ccccc34)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

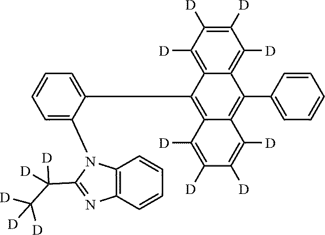 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3ccccc3)c2c1[2H]]

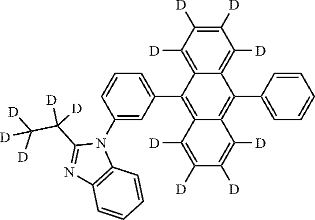 [SMILES: [2H]c1c([2H])c([2H])c2c(-c3cccc(-n4c(C([2H])([2H])C([2H])([2H])[2H])nc5ccccc54)c3)c3c([2H])c([2H])c([2H])c([2H])c3c(-c3ccccc3)c2c1[2H]]

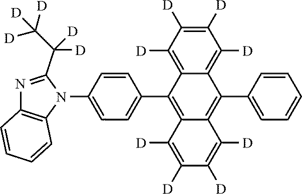 [SMILES: [2H]c1c([2H])c([2H])c2c(-c3ccc(-n4c(C([2H])([2H])C([2H])([2H])[2H])nc5ccccc54)cc3)c3c([2H])c([2H])c([2H])c([2H])c3c(-c3ccccc3)c2c1[2H]]

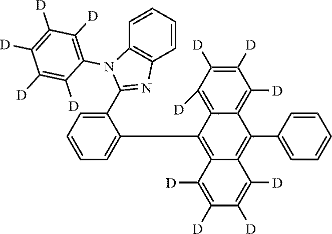 [SMILES: [2H]c1c([2H])c([2H])c(-n2c(-c3ccccc3-c3c4c([2H])c([2H])c([2H])c([2H])c4c(-c4ccccc4)c4c([2H])c([2H])c([2H])c([2H])c34)nc3ccccc32)c([2H])c1[2H]]

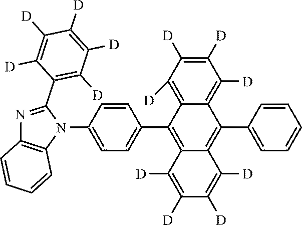 [SMILES: [2H]c1c([2H])c([2H])c(-c2nc3ccccc3n2-c2ccc(-c3c4c([2H])c([2H])c([2H])c([2H])c4c(-c4ccccc4)c4c([2H])c([2H])c([2H])c([2H])c34)cc2)c([2H])c1[2H]]

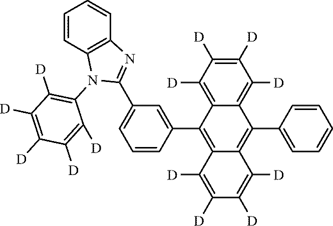 [SMILES: [2H]c1c([2H])c([2H])c(-n2c(-c3cccc(-c4c5c([2H])c([2H])c([2H])c([2H])c5c(-c5ccccc5)c5c([2H])c([2H])c([2H])c([2H])c45)c3)nc3ccccc32)c([2H])c1[2H]]

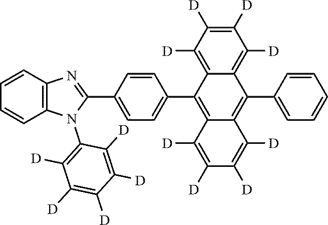 [SMILES: [2H]c1c([2H])c([2H])c(-n2c(-c3ccc(-c4c5c([2H])c([2H])c([2H])c([2H])c5c(-c5ccccc5)c5c([2H])c([2H])c([2H])c([2H])c45)cc3)nc3ccccc32)c([2H])c1[2H]]

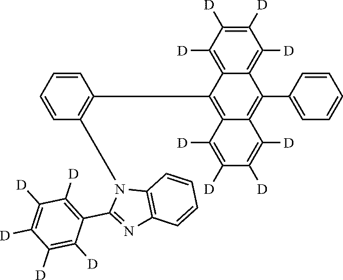 [SMILES: [2H]c1c([2H])c([2H])c(-c2nc3ccccc3n2-c2ccccc2-c2c3c([2H])c([2H])c([2H])c([2H])c3c(-c3ccccc3)c3c([2H])c([2H])c([2H])c([2H])c23)c([2H])c1[2H]]

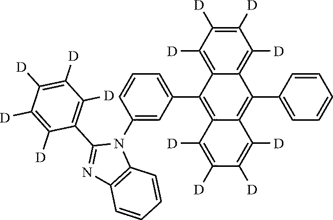 [SMILES: [2H]c1c([2H])c([2H])c(-c2nc3ccccc3n2-c2cccc(-c3c4c([2H])c([2H])c([2H])c([2H])c4c(-c4ccccc4)c4c([2H])c([2H])c([2H])c([2H])c34)c2)c([2H])c1[2H]]

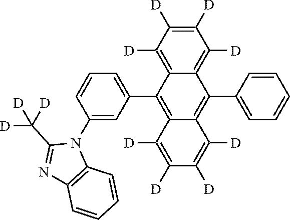 [SMILES: [2H]c1c([2H])c([2H])c2c(-c3cccc(-n4c(C([2H])([2H])[2H])nc5ccccc54)c3)c3c([2H])c([2H])c([2H])c([2H])c3c(-c3ccccc3)c2c1[2H]]

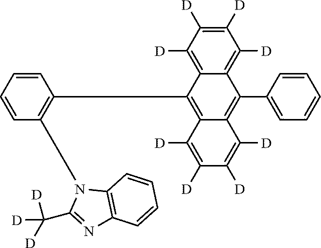 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3ccccc3)c2c1[2H]]

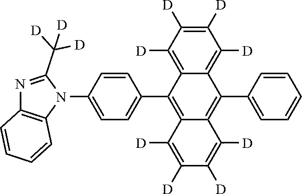 [SMILES: [2H]c1c([2H])c([2H])c2c(-c3ccc(-n4c(C([2H])([2H])[2H])nc5ccccc54)cc3)c3c([2H])c([2H])c([2H])c([2H])c3c(-c3ccccc3)c2c1[2H]]

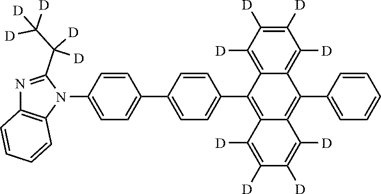 [SMILES: [2H]c1c([2H])c([2H])c2c(-c3ccc(-c4ccc(-n5c(C([2H])([2H])C([2H])([2H])[2H])nc6ccccc65)cc4)cc3)c3c([2H])c([2H])c([2H])c([2H])c3c(-c3ccccc3)c2c1[2H]]

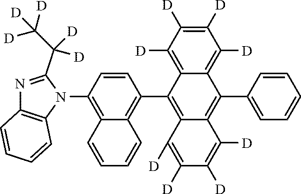 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccccc3)c2c1[2H]]

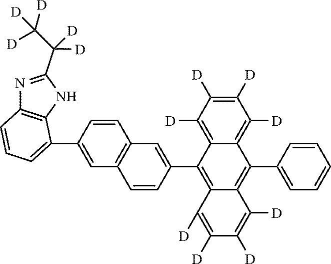 [SMILES: [2H]c1c([2H])c([2H])c2c(-c3ccc4cc(-c5cccc6nc(C([2H])([2H])C([2H])([2H])[2H])[nH]c56)ccc4c3)c3c([2H])c([2H])c([2H])c([2H])c3c(-c3ccccc3)c2c1[2H]]

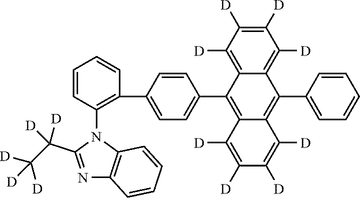 [SMILES: [2H]c1c([2H])c([2H])c2c(-c3ccc(-c4ccccc4-n4c(C([2H])([2H])C([2H])([2H])[2H])nc5ccccc54)cc3)c3c([2H])c([2H])c([2H])c([2H])c3c(-c3ccccc3)c2c1[2H]]

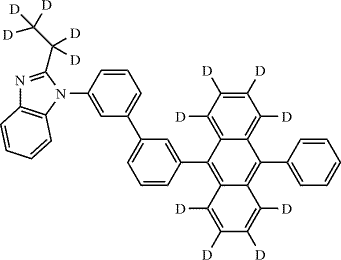 [SMILES: [2H]c1c([2H])c([2H])c2c(-c3cccc(-c4cccc(-n5c(C([2H])([2H])C([2H])([2H])[2H])nc6ccccc65)c4)c3)c3c([2H])c([2H])c([2H])c([2H])c3c(-c3ccccc3)c2c1[2H]]

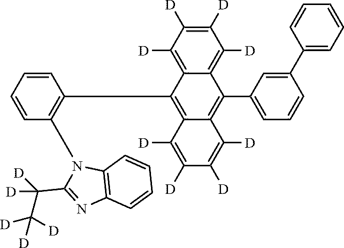 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3cccc(-c4ccccc4)c3)c2c1[2H]]

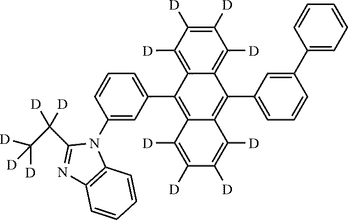 [SMILES: [2H]c1c([2H])c([2H])c2c(-c3cccc(-n4c(C([2H])([2H])C([2H])([2H])[2H])nc5ccccc54)c3)c3c([2H])c([2H])c([2H])c([2H])c3c(-c3cccc(-c4ccccc4)c3)c2c1[2H]]

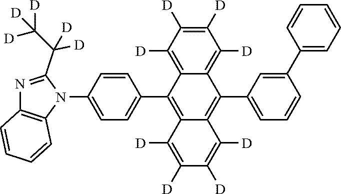 [SMILES: [2H]c1c([2H])c([2H])c2c(-c3cccc(-c4ccccc4)c3)c3c([2H])c([2H])c([2H])c([2H])c3c(-c3ccc(-n4c(C([2H])([2H])C([2H])([2H])[2H])nc5ccccc54)cc3)c2c1[2H]]

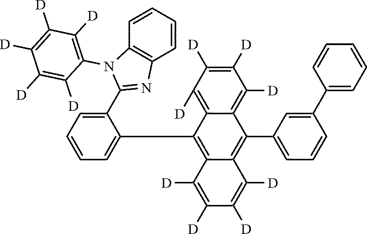 [SMILES: [2H]c1c([2H])c([2H])c(-n2c(-c3ccccc3-c3c4c([2H])c([2H])c([2H])c([2H])c4c(-c4cccc(-c5ccccc5)c4)c4c([2H])c([2H])c([2H])c([2H])c34)nc3ccccc32)c([2H])c1[2H]]

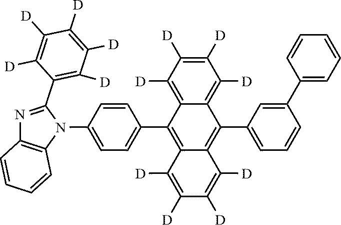 [SMILES: [2H]c1c([2H])c([2H])c(-c2nc3ccccc3n2-c2ccc(-c3c4c([2H])c([2H])c([2H])c([2H])c4c(-c4cccc(-c5ccccc5)c4)c4c([2H])c([2H])c([2H])c([2H])c34)cc2)c([2H])c1[2H]]

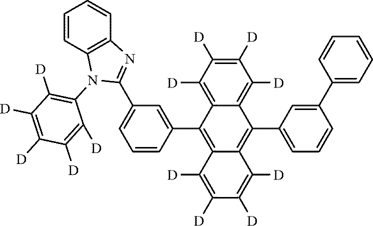 [SMILES: [2H]c1c([2H])c([2H])c(-n2c(-c3cccc(-c4c5c([2H])c([2H])c([2H])c([2H])c5c(-c5cccc(-c6ccccc6)c5)c5c([2H])c([2H])c([2H])c([2H])c45)c3)nc3ccccc32)c([2H])c1[2H]]

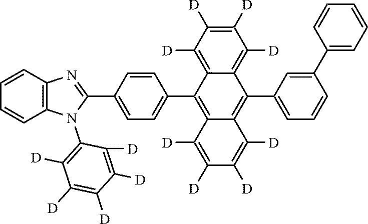 [SMILES: [2H]c1c([2H])c([2H])c(-n2c(-c3ccc(-c4c5c([2H])c([2H])c([2H])c([2H])c5c(-c5cccc(-c6ccccc6)c5)c5c([2H])c([2H])c([2H])c([2H])c45)cc3)nc3ccccc32)c([2H])c1[2H]]

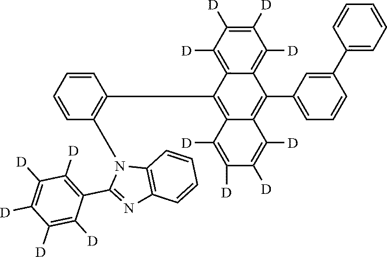 [SMILES: [2H]c1c([2H])c([2H])c(-c2nc3ccccc3n2-c2ccccc2-c2c3c([2H])c([2H])c([2H])c([2H])c3c(-c3cccc(-c4ccccc4)c3)c3c([2H])c([2H])c([2H])c([2H])c23)c([2H])c1[2H]]

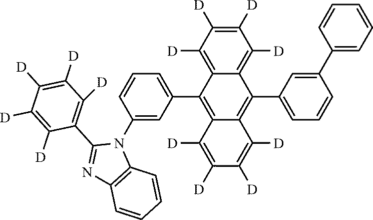 [SMILES: [2H]c1c([2H])c([2H])c(-c2nc3ccccc3n2-c2cccc(-c3c4c([2H])c([2H])c([2H])c([2H])c4c(-c4cccc(-c5ccccc5)c4)c4c([2H])c([2H])c([2H])c([2H])c34)c2)c([2H])c1[2H]]

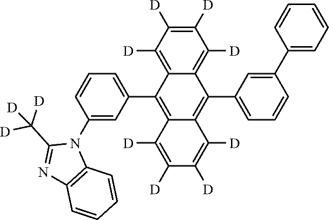 [SMILES: [2H]c1c([2H])c([2H])c2c(-c3cccc(-n4c(C([2H])([2H])[2H])nc5ccccc54)c3)c3c([2H])c([2H])c([2H])c([2H])c3c(-c3cccc(-c4ccccc4)c3)c2c1[2H]]

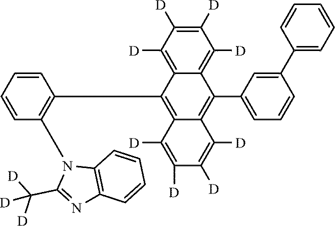 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3cccc(-c4ccccc4)c3)c2c1[2H]]

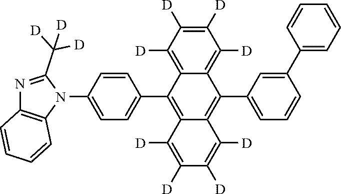 [SMILES: [2H]c1c([2H])c([2H])c2c(-c3cccc(-c4ccccc4)c3)c3c([2H])c([2H])c([2H])c([2H])c3c(-c3ccc(-n4c(C([2H])([2H])[2H])nc5ccccc54)cc3)c2c1[2H]]

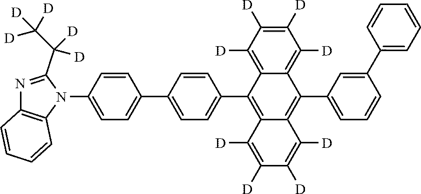 [SMILES: [2H]c1c([2H])c([2H])c2c(-c3cccc(-c4ccccc4)c3)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

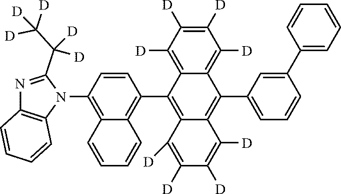 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3cccc(-c4ccccc4)c3)c2c1[2H]]

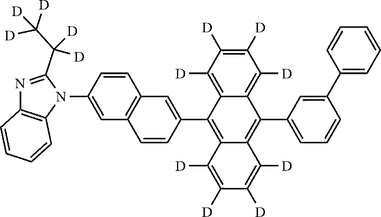 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3cccc(-c4ccccc4)c3)c2c1[2H]]

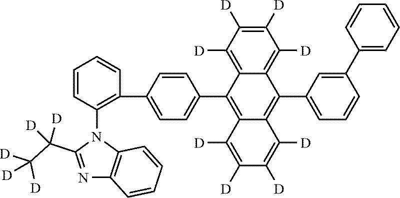 [SMILES: [2H]c1c([2H])c([2H])c2c(-c3cccc(-c4ccccc4)c3)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

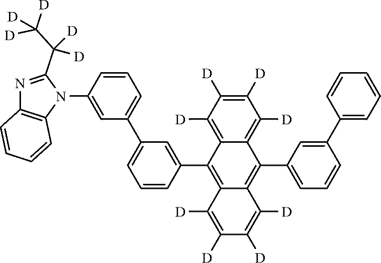 [SMILES: [2H]c1c([2H])c([2H])c2c(-c3cccc(-c4cccc(-n5c(C([2H])([2H])C([2H])([2H])[2H])nc6ccccc65)c4)c3)c3c([2H])c([2H])c([2H])c([2H])c3c(-c3cccc(-c4ccccc4)c3)c2c1[2H]]

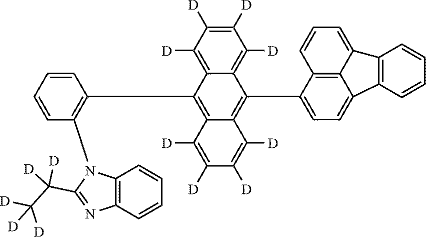 [SMILES: [2H]c1c([2H])c([2H])c2c(-c3ccc4c5c(cccc35)-c3ccccc3-4)c3c([2H])c([2H])c([2H])c([2H])c3c(-c3ccccc3-n3c(C([2H])([2H])C([2H])([2H])[2H])nc4ccccc43)c2c1[2H]]

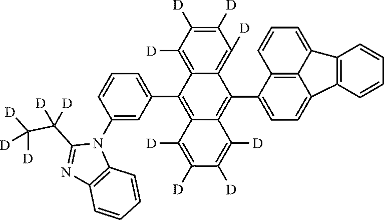 [SMILES: [2H]c1c([2H])c([2H])c2c(-c3ccc4c5c(cccc35)-c3ccccc3-4)c3c([2H])c([2H])c([2H])c([2H])c3c(-c3cccc(-n4c(C([2H])([2H])C([2H])([2H])[2H])nc5ccccc54)c3)c2c1[2H]]

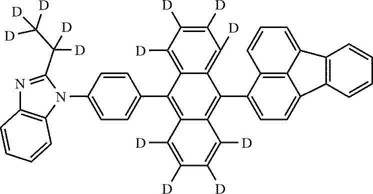 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(C([2H])([2H])C([2H])([2H])[2H])nc5ccccc54)cc3)c2c1[2H]]

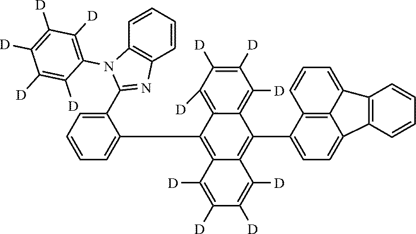 [SMILES: [2H]c1c([2H])c([2H])c(-n2c(-c3ccccc3-c3c4c([2H])c([2H])c([2H])c([2H])c4c(-c4ccc5c6c(cccc46)-c4ccccc4-5)c4c([2H])c([2H])c([2H])c([2H])c34)nc3ccccc32)c([2H])c1[2H]]

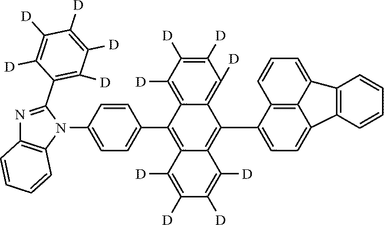 [SMILES: [2H]c1c([2H])c([2H])c(-c2nc3ccccc3n2-c2ccc(-c3c4c([2H])c([2H])c([2H])c([2H])c4c(-c4ccc5c6c(cccc46)-c4ccccc4-5)c4c([2H])c([2H])c([2H])c([2H])c34)cc2)c([2H])c1[2H]]

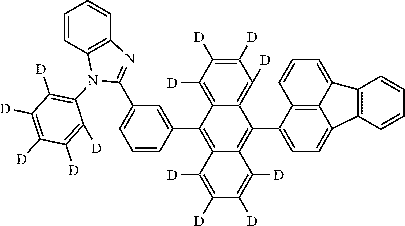 [SMILES: [2H]c1c([2H])c([2H])c(-n2c(-c3cccc(-c4c5c([2H])c([2H])c([2H])c([2H])c5c(-c5ccc6c7c(cccc57)-c5ccccc5-6)c5c([2H])c([2H])c([2H])c([2H])c45)c3)nc3ccccc32)c([2H])c1[2H]]

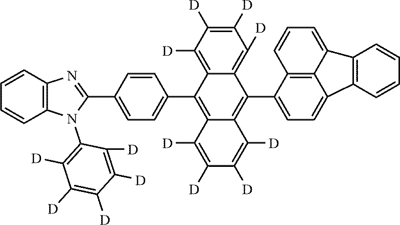 [SMILES: [2H]c1c([2H])c([2H])c(-n2c(-c3ccc(-c4c5c([2H])c([2H])c([2H])c([2H])c5c(-c5ccc6c7c(cccc57)-c5ccccc5-6)c5c([2H])c([2H])c([2H])c([2H])c45)cc3)nc3ccccc32)c([2H])c1[2H]]

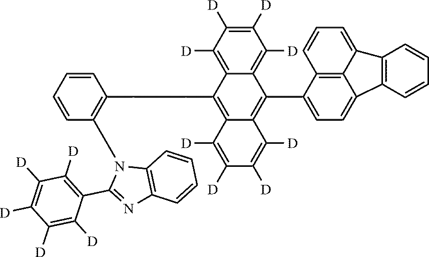 [SMILES: [2H]c1c([2H])c([2H])c(-c2nc3ccccc3n2Cc2ccccc2CCc2c3c([2H])c([2H])c([2H])c([2H])c3c(-c3ccc4c5c(cccc35)-c3ccccc3-4)c3c([2H])c([2H])c([2H])c([2H])c23)c([2H])c1[2H]]

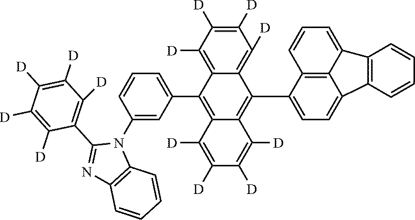 [SMILES: [2H]c1c([2H])c([2H])c(-c2nc3ccccc3n2-c2cccc(-c3c4c([2H])c([2H])c([2H])c([2H])c4c(-c4ccc5c6c(cccc46)-c4ccccc4-5)c4c([2H])c([2H])c([2H])c([2H])c34)c2)c([2H])c1[2H]]

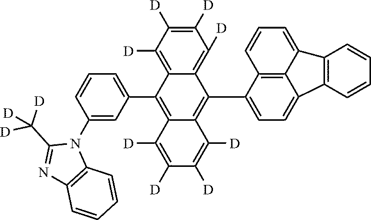 [SMILES: [2H]c1c([2H])c([2H])c2c(-c3ccc4c5c(cccc35)-c3ccccc3-4)c3c([2H])c([2H])c([2H])c([2H])c3c(-c3cccc(-n4c(C([2H])([2H])[2H])nc5ccccc54)c3)c2c1[2H]]

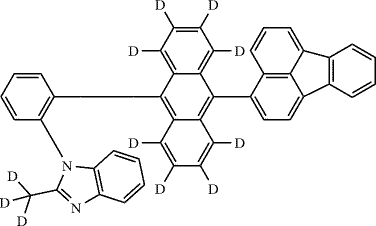 [SMILES: [2H]c1c([2H])c([2H])c2c(-c3ccc4c5c(cccc35)-c3ccccc3-4)c3c([2H])c([2H])c([2H])c([2H])c3c(CCc3ccccc3Cn3c(C([2H])([2H])[2H])nc4ccccc43)c2c1[2H]]

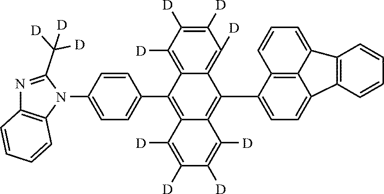 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(C([2H])([2H])[2H])nc5ccccc54)cc3)c2c1[2H]]

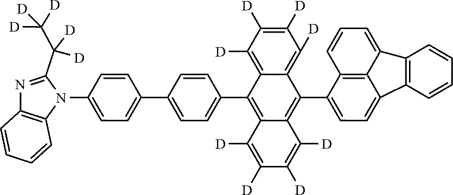 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

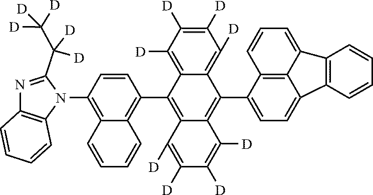 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(C([2H])([2H])C([2H])([2H])[2H])nc5ccccc54)c4ccccc34)c2c1[2H]]

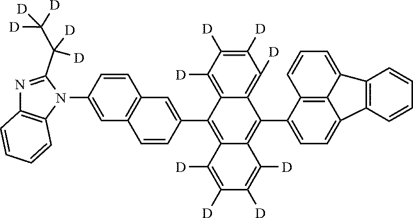 [SMILES: [2H]c1c([2H])c([2H])c2c(-c3ccc4c5c(cccc35)-c3ccccc3-4)c3c([2H])c([2H])c([2H])c([2H])c3c(-c3ccc4cc(-n5c(C([2H])([2H])C([2H])([2H])[2H])nc6ccccc65)ccc4c3)c2c1[2H]]

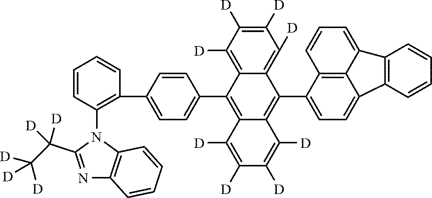 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

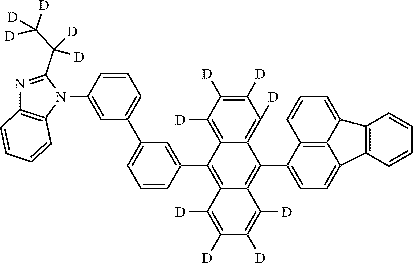 [SMILES: [2H]c1c([2H])c([2H])c2c(-c3ccc4c5c(cccc35)-c3ccccc3-4)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

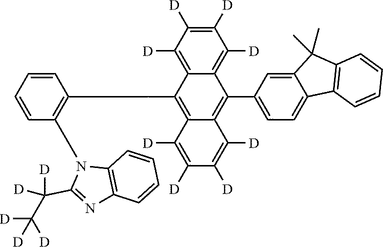 [SMILES: [2H]c1c([2H])c([2H])c2c(-c3ccc4c(c3)C(C)(C)c3ccccc3-4)c3c([2H])c([2H])c([2H])c([2H])c3c(CCc3ccccc3Cn3c(C([2H])([2H])C([2H])([2H])[2H])nc4ccccc43)c2c1[2H]]

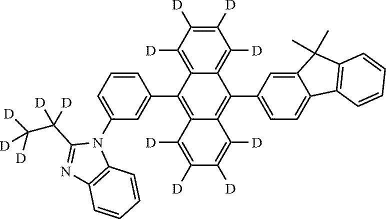 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-n4c(C([2H])([2H])C([2H])([2H])[2H])nc5ccccc54)c3)c2c1[2H]]

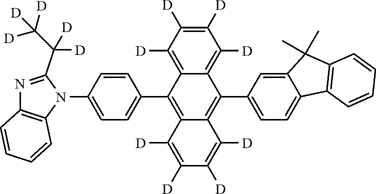 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-n4c(C([2H])([2H])C([2H])([2H])[2H])nc5ccccc54)cc3)c2c1[2H]]

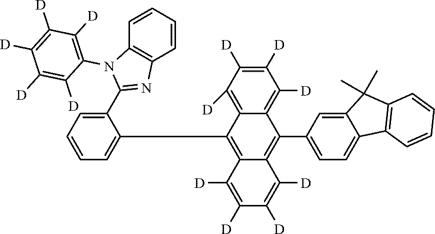 [SMILES: [2H]c1c([2H])c([2H])c(-n2c(-c3ccccc3-c3c4c([2H])c([2H])c([2H])c([2H])c4c(-c4ccc5c(c4)C(C)(C)c4ccccc4-5)c4c([2H])c([2H])c([2H])c([2H])c34)nc3ccccc32)c([2H])c1[2H]]

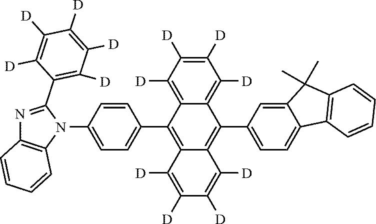 [SMILES: [2H]c1c([2H])c([2H])c(-c2nc3ccccc3n2-c2ccc(-c3c4c([2H])c([2H])c([2H])c([2H])c4c(-c4ccc5c(c4)C(C)(C)c4ccccc4-5)c4c([2H])c([2H])c([2H])c([2H])c34)cc2)c([2H])c1[2H]]

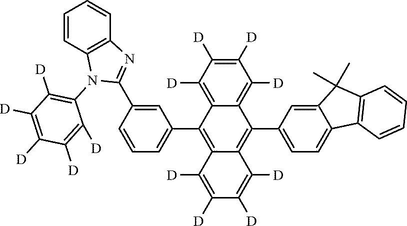 [SMILES: [2H]c1c([2H])c([2H])c(-n2c(-c3cccc(-c4c5c([2H])c([2H])c([2H])c([2H])c5c(-c5ccc6c(c5)C(C)(C)c5ccccc5-6)c5c([2H])c([2H])c([2H])c([2H])c45)c3)nc3ccccc32)c([2H])c1[2H]]

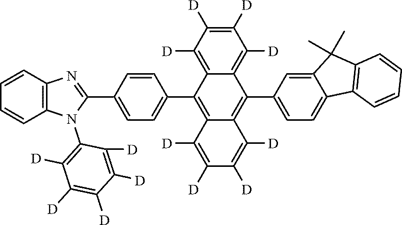 [SMILES: [2H]c1c([2H])c([2H])c(-n2c(-c3ccc(-c4c5c([2H])c([2H])c([2H])c([2H])c5c(-c5ccc6c(c5)C(C)(C)c5ccccc5-6)c5c([2H])c([2H])c([2H])c([2H])c45)cc3)nc3ccccc32)c([2H])c1[2H]]

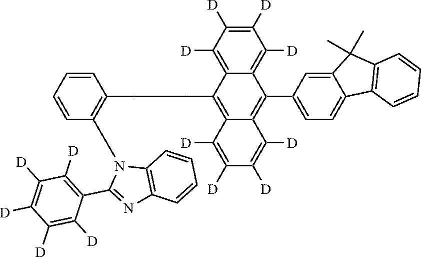 [SMILES: [2H]c1c([2H])c([2H])c(-c2nc3ccccc3n2Cc2ccccc2CCc2c3c([2H])c([2H])c([2H])c([2H])c3c(-c3ccc4c(c3)C(C)(C)c3ccccc3-4)c3c([2H])c([2H])c([2H])c([2H])c23)c([2H])c1[2H]]

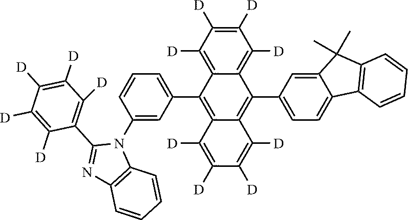 [SMILES: [2H]c1c([2H])c([2H])c(-c2nc3ccccc3n2-c2cccc(-c3c4c([2H])c([2H])c([2H])c([2H])c4c(-c4ccc5c(c4)C(C)(C)c4ccccc4-5)c4c([2H])c([2H])c([2H])c([2H])c34)c2)c([2H])c1[2H]]

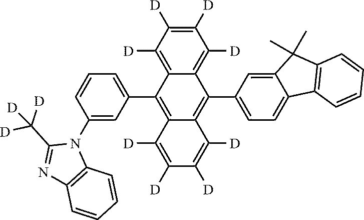 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-n4c(C([2H])([2H])[2H])nc5ccccc54)c3)c2c1[2H]]

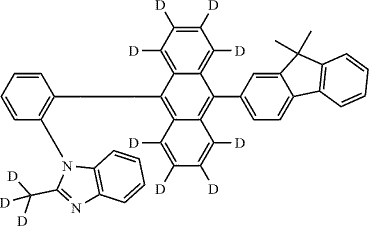 [SMILES: [2H]c1c([2H])c([2H])c2c(-c3ccc4c(c3)C(C)(C)c3ccccc3-4)c3c([2H])c([2H])c([2H])c([2H])c3c(CCc3ccccc3Cn3c(C([2H])([2H])[2H])nc4ccccc43)c2c1[2H]]

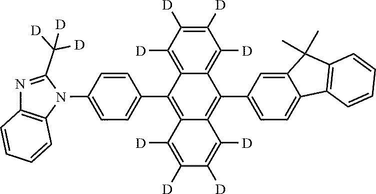 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-n4c(C([2H])([2H])[2H])nc5ccccc54)cc3)c2c1[2H]]

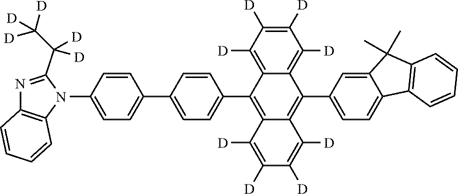 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

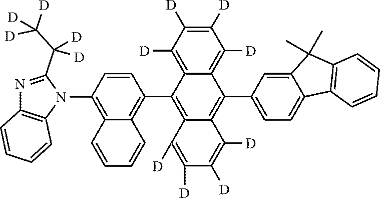 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3ccc4c(c3)C(C)(C)c3ccccc3-4)c2c1[2H]]

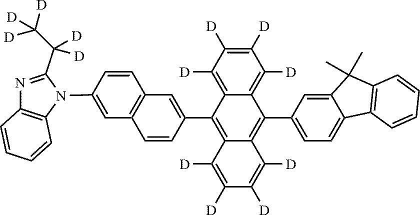 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3ccc4c(c3)C(C)(C)c3ccccc3-4)c2c1[2H]]

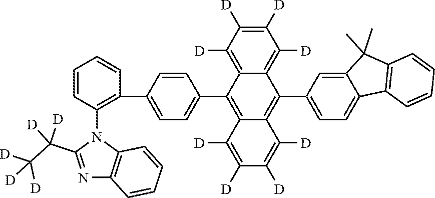 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

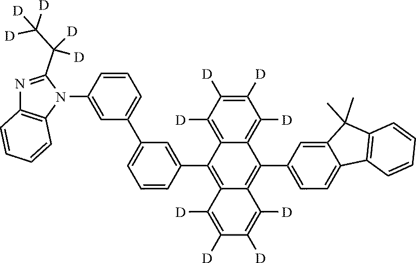 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

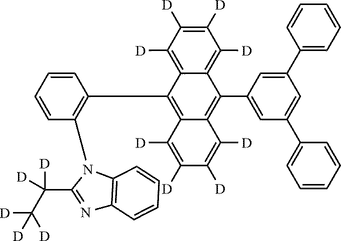 [SMILES: [2H]c1c([2H])c([2H])c2c(-c3ccccc3-n3c(C([2H])([2H])C([2H])([2H])[2H])nc4ccccc43)c3c([2H])c([2H])c([2H])c([2H])c3c(-c3cc(-c4ccccc4)cc(-c4ccccc4)c3)c2c1[2H]]

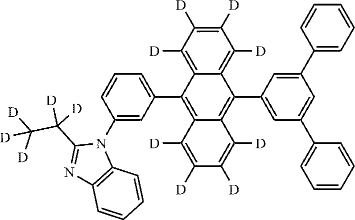 [SMILES: [2H]c1c([2H])c([2H])c2c(-c3cccc(-n4c(C([2H])([2H])C([2H])([2H])[2H])nc5ccccc54)c3)c3c([2H])c([2H])c([2H])c([2H])c3c(-c3cc(-c4ccccc4)cc(-c4ccccc4)c3)c2c1[2H]]

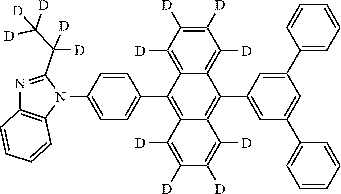 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-n4c(C([2H])([2H])C([2H])([2H])[2H])nc5ccccc54)cc3)c2c1[2H]]

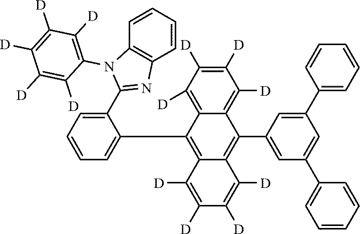 [SMILES: [2H]c1c([2H])c([2H])c(-n2c(-c3ccccc3-c3c4c([2H])c([2H])c([2H])c([2H])c4c(-c4cc(-c5ccccc5)cc(-c5ccccc5)c4)c4c([2H])c([2H])c([2H])c([2H])c34)nc3ccccc32)c([2H])c1[2H]]

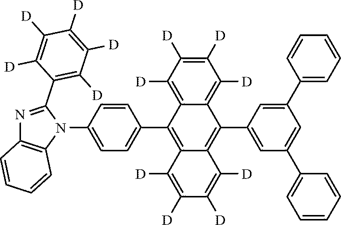 [SMILES: [2H]c1c([2H])c([2H])c(-c2nc3ccccc3n2-c2ccc(-c3c4c([2H])c([2H])c([2H])c([2H])c4c(-c4cc(-c5ccccc5)cc(-c5ccccc5)c4)c4c([2H])c([2H])c([2H])c([2H])c34)cc2)c([2H])c1[2H]]

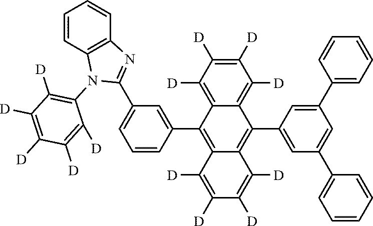 [SMILES: [2H]c1c([2H])c([2H])c(-n2c(-c3cccc(-c4c5c([2H])c([2H])c([2H])c([2H])c5c(-c5cc(-c6ccccc6)cc(-c6ccccc6)c5)c5c([2H])c([2H])c([2H])c([2H])c45)c3)nc3ccccc32)c([2H])c1[2H]]

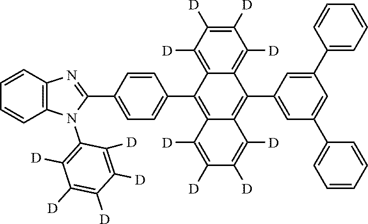 [SMILES: [2H]c1c([2H])c([2H])c(-n2c(-c3ccc(-c4c5c([2H])c([2H])c([2H])c([2H])c5c(-c5cc(-c6ccccc6)cc(-c6ccccc6)c5)c5c([2H])c([2H])c([2H])c([2H])c45)cc3)nc3ccccc32)c([2H])c1[2H]]

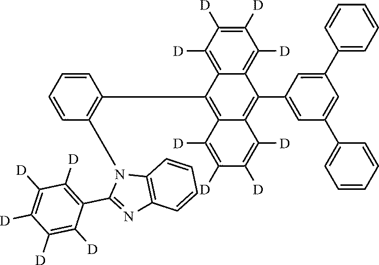 [SMILES: [2H]c1c([2H])c([2H])c(-c2nc3ccccc3n2-c2ccccc2-c2c3c([2H])c([2H])c([2H])c([2H])c3c(-c3cc(-c4ccccc4)cc(-c4ccccc4)c3)c3c([2H])c([2H])c([2H])c([2H])c23)c([2H])c1[2H]]

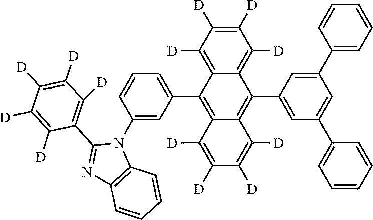 [SMILES: [2H]c1c([2H])c([2H])c(-c2nc3ccccc3n2-c2cccc(-c3c4c([2H])c([2H])c([2H])c([2H])c4c(-c4cc(-c5ccccc5)cc(-c5ccccc5)c4)c4c([2H])c([2H])c([2H])c([2H])c34)c2)c([2H])c1[2H]]

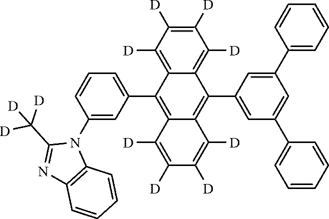 [SMILES: [2H]c1c([2H])c([2H])c2c(-c3cccc(-n4c(C([2H])([2H])[2H])nc5ccccc54)c3)c3c([2H])c([2H])c([2H])c([2H])c3c(-c3cc(-c4ccccc4)cc(-c4ccccc4)c3)c2c1[2H]]

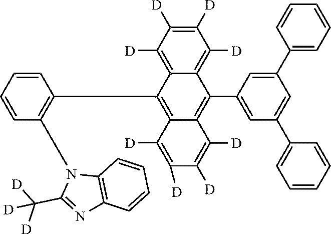 [SMILES: [2H]c1c([2H])c([2H])c2c(-c3ccccc3-n3c(C([2H])([2H])[2H])nc4ccccc43)c3c([2H])c([2H])c([2H])c([2H])c3c(-c3cc(-c4ccccc4)cc(-c4ccccc4)c3)c2c1[2H]]

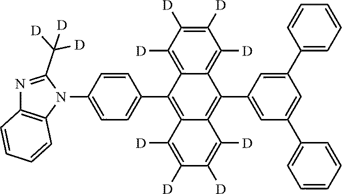 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-n4c(C([2H])([2H])[2H])nc5ccccc54)cc3)c2c1[2H]]

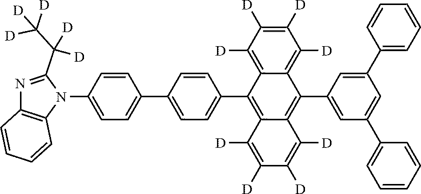 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4ccc(-n5c(C([2H])([2H])C([2H])([2H])[2H])nc6ccccc65)cc4)cc3)c2c1[2H]]

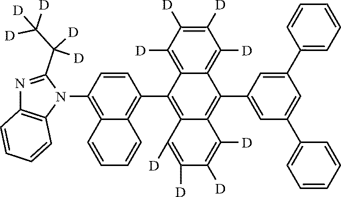 [SMILES: [2H]c1c([2H])c([2H])c2c(-c3ccc(-n4c(C([2H])([2H])C([2H])([2H])[2H])nc5ccccc54)c4ccccc34)c3c([2H])c([2H])c([2H])c([2H])c3c(-c3cc(-c4ccccc4)cc(-c4ccccc4)c3)c2c1[2H]]

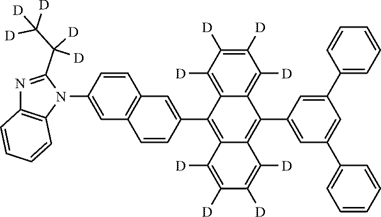 [SMILES: [2H]c1c([2H])c([2H])c2c(-c3ccc4cc(-n5c(C([2H])([2H])C([2H])([2H])[2H])nc6ccccc65)ccc4c3)c3c([2H])c([2H])c([2H])c([2H])c3c(-c3cc(-c4ccccc4)cc(-c4ccccc4)c3)c2c1[2H]]

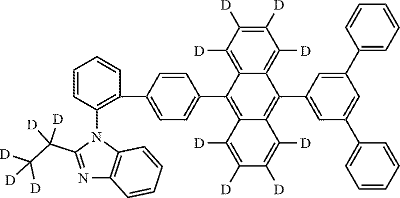 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4ccccc4-n4c(C([2H])([2H])C([2H])([2H])[2H])nc5ccccc54)cc3)c2c1[2H]]

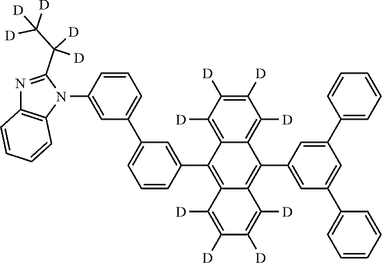 [SMILES: [2H]c1c([2H])c([2H])c2c(-c3cc(-c4ccccc4)cc(-c4ccccc4)c3)c3c([2H])c([2H])c([2H])c([2H])c3c(-c3cccc(-c4cccc(-n5c(C([2H])([2H])C([2H])([2H])[2H])nc6ccccc65)c4)c3)c2c1[2H]]

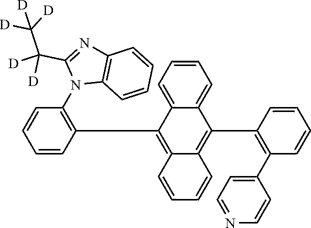 [SMILES: [2H]C([2H])([2H])C([2H])([2H])c1nc2ccccc2n1-c1ccccc1-c1c2ccccc2c(-c2ccccc2-c2ccncc2)c2ccccc12]

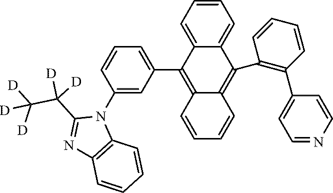 [SMILES: [2H]C([2H])([2H])C([2H])([2H])c1nc2ccccc2n1-c1cccc(-c2c3ccccc3c(-c3ccccc3-c3ccncc3)c3ccccc23)c1]

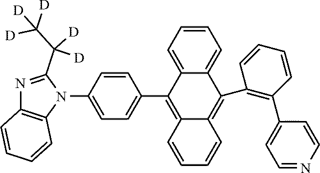 [SMILES: [2H]C([2H])([2H])C([2H])([2H])c1nc2ccccc2n1-c1ccc(-c2c3ccccc3c(-c3ccccc3-c3ccncc3)c3ccccc23)cc1]

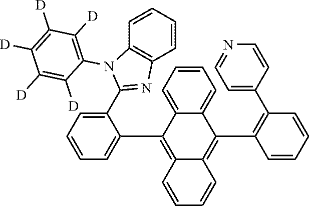 [SMILES: [2H]c1c([2H])c([2H])c(-n2c(-c3ccccc3-c3c4ccccc4c(-c4ccccc4-c4ccncc4)c4ccccc34)nc3ccccc32)c([2H])c1[2H]]

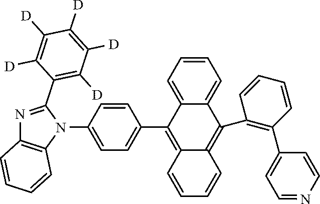 [SMILES: [2H]c1c([2H])c([2H])c(-c2nc3ccccc3n2-c2ccc(-c3c4ccccc4c(-c4ccccc4-c4ccncc4)c4ccccc34)cc2)c([2H])c1[2H]]

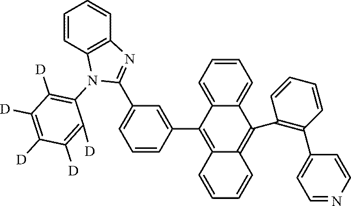 [SMILES: [2H]c1c([2H])c([2H])c(-n2c(-c3cccc(-c4c5ccccc5c(-c5ccccc5-c5ccncc5)c5ccccc45)c3)nc3ccccc32)c([2H])c1[2H]]

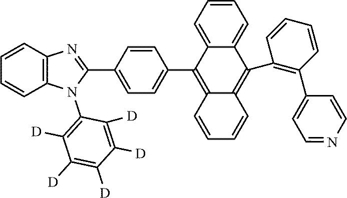 [SMILES: [2H]c1c([2H])c([2H])c(-n2c(-c3ccc(-c4c5ccccc5c(-c5ccccc5-c5ccncc5)c5ccccc45)cc3)nc3ccccc32)c([2H])c1[2H]]

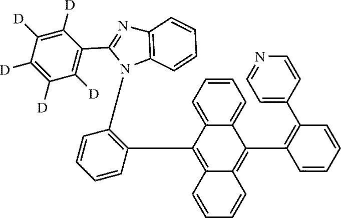 [SMILES: [2H]c1c([2H])c([2H])c(-c2nc3ccccc3n2-c2ccccc2-c2c3ccccc3c(-c3ccccc3-c3ccncc3)c3ccccc23)c([2H])c1[2H]]

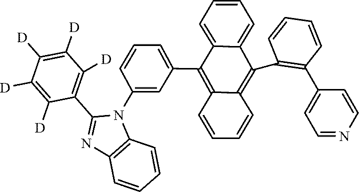 [SMILES: [2H]c1c([2H])c([2H])c(-c2nc3ccccc3n2-c2cccc(-c3c4ccccc4c(-c4ccccc4-c4ccncc4)c4ccccc34)c2)c([2H])c1[2H]]

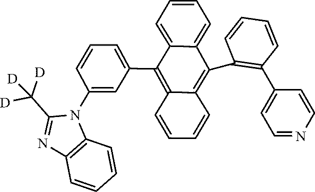 [SMILES: [2H]C([2H])([2H])c1nc2ccccc2n1-c1cccc(-c2c3ccccc3c(-c3ccccc3-c3ccncc3)c3ccccc23)c1]

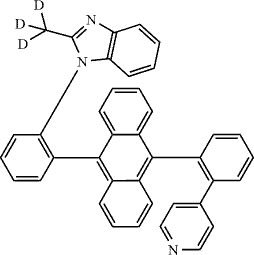 [SMILES: [2H]C([2H])([2H])c1nc2ccccc2n1-c1ccccc1-c1c2ccccc2c(-c2ccccc2-c2ccncc2)c2ccccc12]

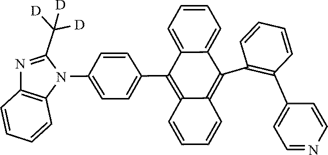 [SMILES: [2H]C([2H])([2H])c1nc2ccccc2n1-c1ccc(-c2c3ccccc3c(-c3ccccc3-c3ccncc3)c3ccccc23)cc1]

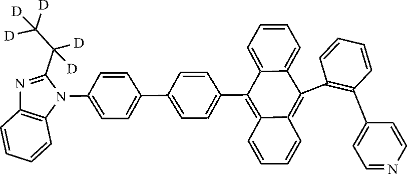 [SMILES: [2H]C([2H])([2H])C([2H])([2H])c1nc2ccccc2n1-c1ccc(-c2ccc(-c3c4ccccc4c(-c4ccccc4-c4ccncc4)c4ccccc34)cc2)cc1]

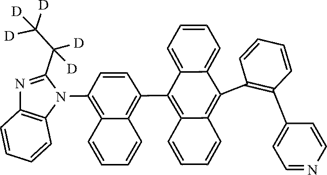 [SMILES: [2H]C([2H])([2H])C([2H])([2H])c1nc2ccccc2n1-c1ccc(-c2c3ccccc3c(-c3ccccc3-c3ccncc3)c3ccccc23)c2ccccc12]

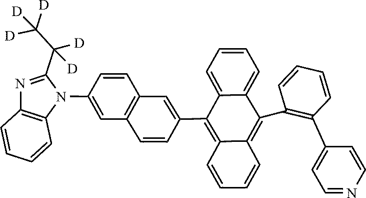 [SMILES: [2H]C([2H])([2H])C([2H])([2H])c1nc2ccccc2n1-c1ccc2cc(-c3c4ccccc4c(-c4ccccc4-c4ccncc4)c4ccccc34)ccc2c1]

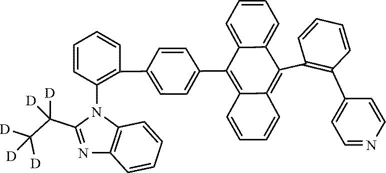 [SMILES: [2H]C([2H])([2H])C([2H])([2H])c1nc2ccccc2n1-c1ccccc1-c1ccc(-c2c3ccccc3c(-c3ccccc3-c3ccncc3)c3ccccc23)cc1]

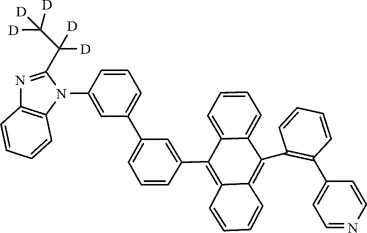 [SMILES: [2H]C([2H])([2H])C([2H])([2H])c1nc2ccccc2n1-c1cccc(-c2cccc(-c3c4ccccc4c(-c4ccccc4-c4ccncc4)c4ccccc34)c2)c1]

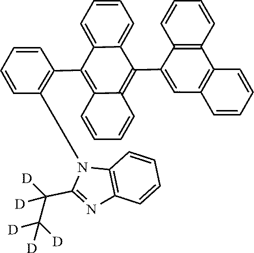 [SMILES: [2H]C([2H])([2H])C([2H])([2H])c1nc2ccccc2n1-c1ccccc1-c1c2ccccc2c(-c2cc3ccccc3c3ccccc23)c2ccccc12]

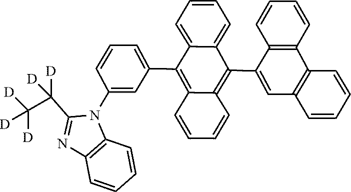 [SMILES: [2H]C([2H])([2H])C([2H])([2H])c1nc2ccccc2n1-c1cccc(-c2c3ccccc3c(-c3cc4ccccc4c4ccccc34)c3ccccc23)c1]

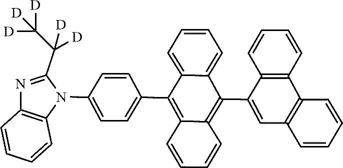 [SMILES: [2H]C([2H])([2H])C([2H])([2H])c1nc2ccccc2n1-c1ccc(-c2c3ccccc3c(-c3cc4ccccc4c4ccccc34)c3ccccc23)cc1]

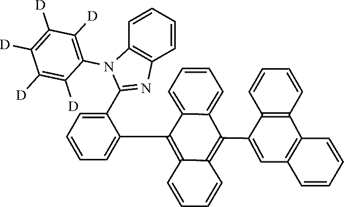 [SMILES: [2H]c1c([2H])c([2H])c(-n2c(-c3ccccc3-c3c4ccccc4c(-c4cc5ccccc5c5ccccc45)c4ccccc34)nc3ccccc32)c([2H])c1[2H]]

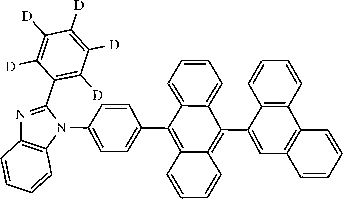 [SMILES: [2H]c1c([2H])c([2H])c(-c2nc3ccccc3n2-c2ccc(-c3c4ccccc4c(-c4cc5ccccc5c5ccccc45)c4ccccc34)cc2)c([2H])c1[2H]]

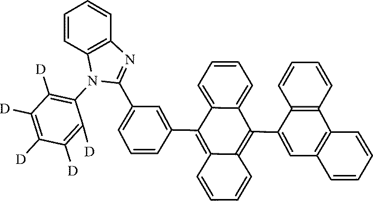 [SMILES: [2H]c1c([2H])c([2H])c(-n2c(-c3cccc(-c4c5ccccc5c(-c5cc6ccccc6c6ccccc56)c5ccccc45)c3)nc3ccccc32)c([2H])c1[2H]]

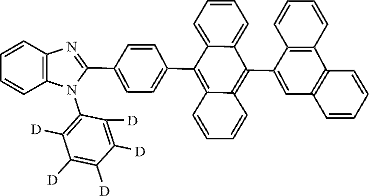 [SMILES: [2H]c1c([2H])c([2H])c(-n2c(-c3ccc(-c4c5ccccc5c(-c5cc6ccccc6c6ccccc56)c5ccccc45)cc3)nc3ccccc32)c([2H])c1[2H]]

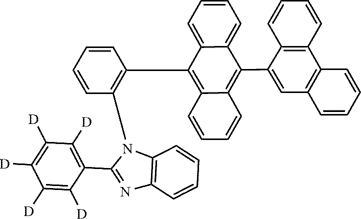 [SMILES: [2H]c1c([2H])c([2H])c(-c2nc3ccccc3n2-c2ccccc2-c2c3ccccc3c(-c3cc4ccccc4c4ccccc34)c3ccccc23)c([2H])c1[2H]]

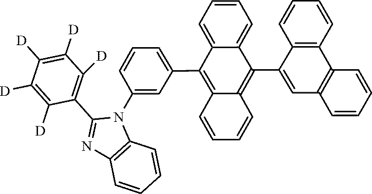 [SMILES: [2H]c1c([2H])c([2H])c(-c2nc3ccccc3n2-c2cccc(-c3c4ccccc4c(-c4cc5ccccc5c5ccccc45)c4ccccc34)c2)c([2H])c1[2H]]

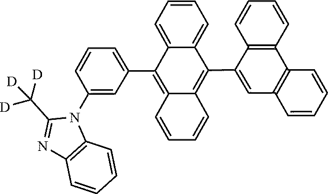 [SMILES: [2H]C([2H])([2H])c1nc2ccccc2n1-c1cccc(-c2c3ccccc3c(-c3cc4ccccc4c4ccccc34)c3ccccc23)c1]

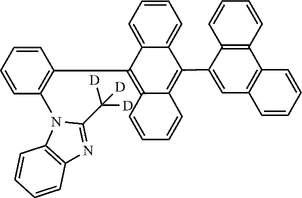 [SMILES: [2H]C([2H])([2H])c1nc2ccccc2n1-c1ccccc1-c1c2ccccc2c(-c2cc3ccccc3c3ccccc23)c2ccccc12]

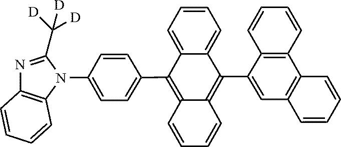 [SMILES: [2H]C([2H])([2H])c1nc2ccccc2n1-c1ccc(-c2c3ccccc3c(-c3cc4ccccc4c4ccccc34)c3ccccc23)cc1]

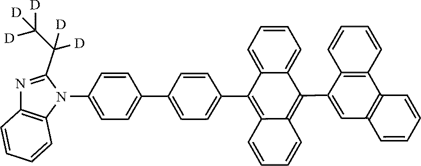 [SMILES: [2H]C([2H])([2H])C([2H])([2H])c1nc2ccccc2n1-c1ccc(-c2ccc(-c3c4ccccc4c(-c4cc5ccccc5c5ccccc45)c4ccccc34)cc2)cc1]

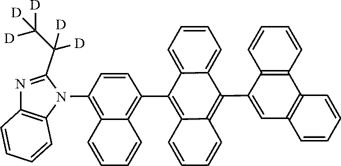 [SMILES: [2H]C([2H])([2H])C([2H])([2H])c1nc2ccccc2n1-c1ccc(-c2c3ccccc3c(-c3cc4ccccc4c4ccccc34)c3ccccc23)c2ccccc12]

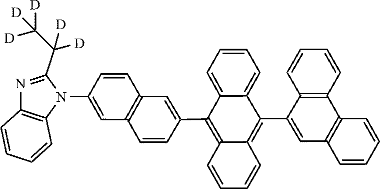 [SMILES: [2H]C([2H])([2H])C([2H])([2H])c1nc2ccccc2n1-c1ccc2cc(-c3c4ccccc4c(-c4cc5ccccc5c5ccccc45)c4ccccc34)ccc2c1]

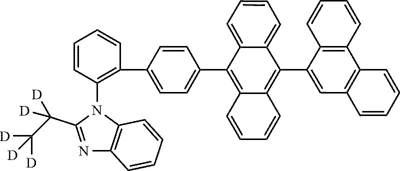 [SMILES: [2H]C([2H])([2H])C([2H])([2H])c1nc2ccccc2n1-c1ccccc1-c1ccc(-c2c3ccccc3c(-c3cc4ccccc4c4ccccc34)c3ccccc23)cc1]

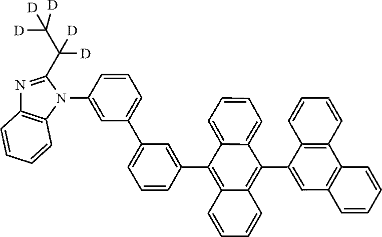 [SMILES: [2H]C([2H])([2H])C([2H])([2H])c1nc2ccccc2n1-c1cccc(-c2cccc(-c3c4ccccc4c(-c4cc5ccccc5c5ccccc45)c4ccccc34)c2)c1]

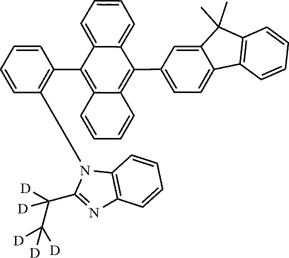 [SMILES: [2H]C([2H])([2H])C([2H])([2H])c1nc2ccccc2n1-c1ccccc1-c1c2ccccc2c(-c2ccc3c(c2)C(C)(C)c2ccccc2-3)c2ccccc12]

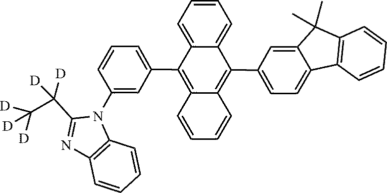 [SMILES: [2H]C([2H])([2H])C([2H])([2H])c1nc2ccccc2n1-c1cccc(-c2c3ccccc3c(-c3ccc4c(c3)C(C)(C)c3ccccc3-4)c3ccccc23)c1]

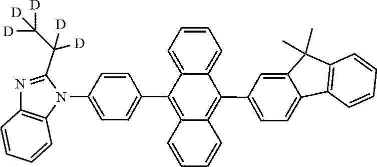 [SMILES: [2H]C([2H])([2H])C([2H])([2H])c1nc2ccccc2n1-c1ccc(-c2c3ccccc3c(-c3ccc4c(c3)C(C)(C)c3ccccc3-4)c3ccccc23)cc1]

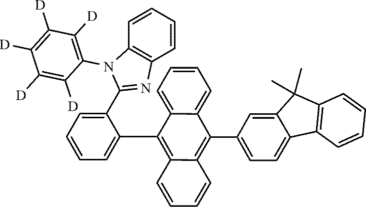 [SMILES: [2H]c1c([2H])c([2H])c(-n2c(-c3ccccc3-c3c4ccccc4c(-c4ccc5c(c4)C(C)(C)c4ccccc4-5)c4ccccc34)nc3ccccc32)c([2H])c1[2H]]

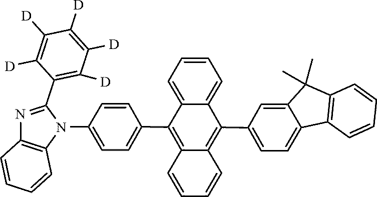 [SMILES: [2H]c1c([2H])c([2H])c(-c2nc3ccccc3n2-c2ccc(-c3c4ccccc4c(-c4ccc5c(c4)C(C)(C)c4ccccc4-5)c4ccccc34)cc2)c([2H])c1[2H]]

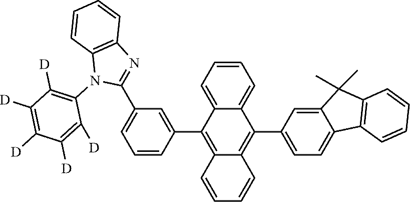 [SMILES: [2H]c1c([2H])c([2H])c(-n2c(-c3cccc(-c4c5ccccc5c(-c5ccc6c(c5)C(C)(C)c5ccccc5-6)c5ccccc45)c3)nc3ccccc32)c([2H])c1[2H]]

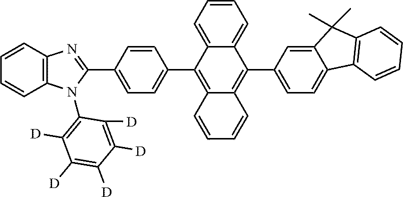 [SMILES: [2H]c1c([2H])c([2H])c(-n2c(-c3ccc(-c4c5ccccc5c(-c5ccc6c(c5)C(C)(C)c5ccccc5-6)c5ccccc45)cc3)nc3ccccc32)c([2H])c1[2H]]

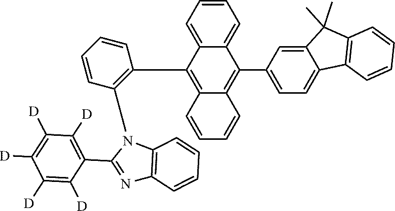 [SMILES: [2H]c1c([2H])c([2H])c(-c2nc3ccccc3n2-c2ccccc2-c2c3ccccc3c(-c3ccc4c(c3)C(C)(C)c3ccccc3-4)c3ccccc23)c([2H])c1[2H]]

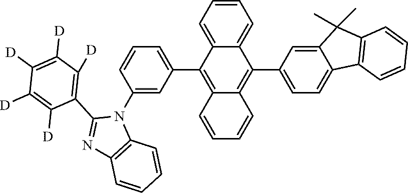 [SMILES: [2H]c1c([2H])c([2H])c(-c2nc3ccccc3n2-c2cccc(-c3c4ccccc4c(-c4ccc5c(c4)C(C)(C)c4ccccc4-5)c4ccccc34)c2)c([2H])c1[2H]]

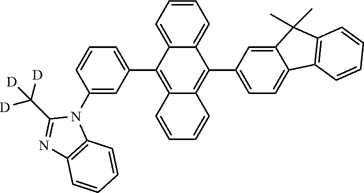 [SMILES: [2H]C([2H])([2H])c1nc2ccccc2n1-c1cccc(-c2c3ccccc3c(-c3ccc4c(c3)C(C)(C)c3ccccc3-4)c3ccccc23)c1]

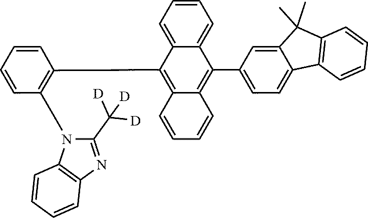 [SMILES: [2H]C([2H])([2H])c1nc2ccccc2n1Cc1ccccc1Cc1c2ccccc2c(-c2ccc3c(c2)C(C)(C)c2ccccc2-3)c2ccccc12]

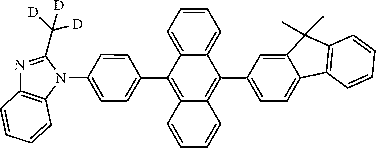 [SMILES: [2H]C([2H])([2H])c1nc2ccccc2n1-c1ccc(-c2c3ccccc3c(-c3ccc4c(c3)C(C)(C)c3ccccc3-4)c3ccccc23)cc1]

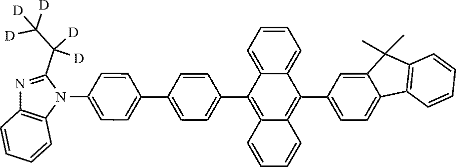 [SMILES: [2H]C([2H])([2H])C([2H])([2H])c1nc2ccccc2n1-c1ccc(-c2ccc(-c3c4ccccc4c(-c4ccc5c(c4)C(C)(C)c4ccccc4-5)c4ccccc34)cc2)cc1]

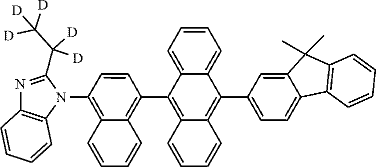 [SMILES: [2H]C([2H])([2H])C([2H])([2H])c1nc2ccccc2n1-c1ccc(-c2c3ccccc3c(-c3ccc4c(c3)C(C)(C)c3ccccc3-4)c3ccccc23)c2ccccc12]

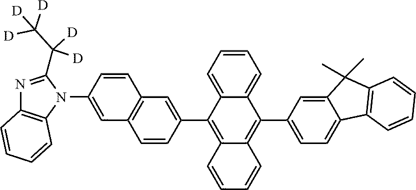 [SMILES: [2H]C([2H])([2H])C([2H])([2H])c1nc2ccccc2n1-c1ccc2cc(-c3c4ccccc4c(-c4ccc5c(c4)C(C)(C)c4ccccc4-5)c4ccccc34)ccc2c1]

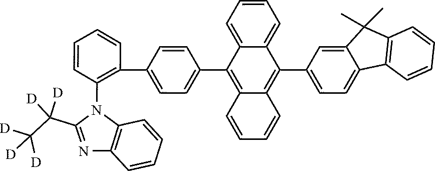 [SMILES: [2H]C([2H])([2H])C([2H])([2H])c1nc2ccccc2n1-c1ccccc1-c1ccc(-c2c3ccccc3c(-c3ccc4c(c3)C(C)(C)c3ccccc3-4)c3ccccc23)cc1]

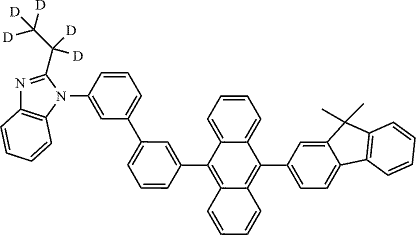 [SMILES: [2H]C([2H])([2H])C([2H])([2H])c1nc2ccccc2n1-c1cccc(-c2cccc(-c3c4ccccc4c(-c4ccc5c(c4)C(C)(C)c4ccccc4-5)c4ccccc34)c2)c1]

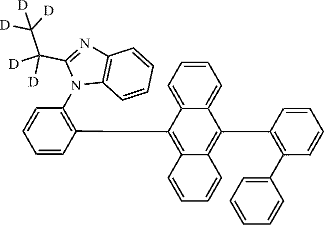 [SMILES: [2H]C([2H])([2H])C([2H])([2H])c1nc2ccccc2n1-c1ccccc1-c1c2ccccc2c(-c2ccccc2-c2ccccc2)c2ccccc12]

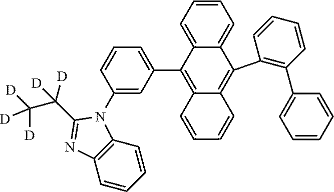 [SMILES: [2H]C([2H])([2H])C([2H])([2H])c1nc2ccccc2n1-c1cccc(-c2c3ccccc3c(-c3ccccc3-c3ccccc3)c3ccccc23)c1]

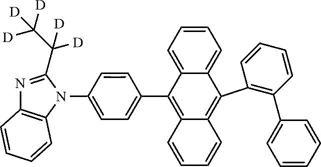 [SMILES: [2H]C([2H])([2H])C([2H])([2H])c1nc2ccccc2n1-c1ccc(-c2c3ccccc3c(-c3ccccc3-c3ccccc3)c3ccccc23)cc1]

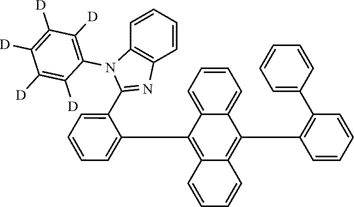 [SMILES: [2H]c1c([2H])c([2H])c(-n2c(-c3ccccc3-c3c4ccccc4c(-c4ccccc4-c4ccccc4)c4ccccc34)nc3ccccc32)c([2H])c1[2H]]

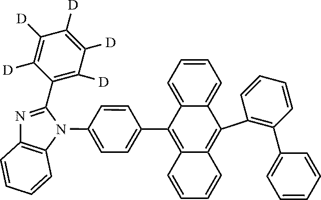 [SMILES: [2H]c1c([2H])c([2H])c(-c2nc3ccccc3n2-c2ccc(-c3c4ccccc4c(-c4ccccc4-c4ccccc4)c4ccccc34)cc2)c([2H])c1[2H]]

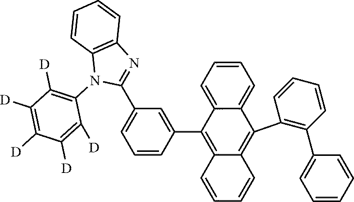 [SMILES: [2H]c1c([2H])c([2H])c(-n2c(-c3cccc(-c4c5ccccc5c(-c5ccccc5-c5ccccc5)c5ccccc45)c3)nc3ccccc32)c([2H])c1[2H]]

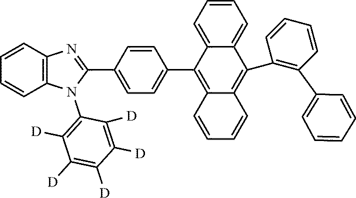 [SMILES: [2H]c1c([2H])c([2H])c(-n2c(-c3ccc(-c4c5ccccc5c(-c5ccccc5-c5ccccc5)c5ccccc45)cc3)nc3ccccc32)c([2H])c1[2H]]

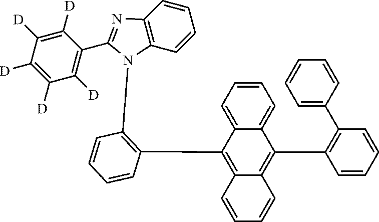 [SMILES: [2H]c1c([2H])c([2H])c(-c2nc3ccccc3n2-c2ccccc2-c2c3ccccc3c(-c3ccccc3-c3ccccc3)c3ccccc23)c([2H])c1[2H]]

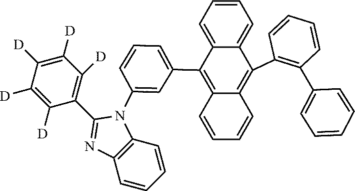 [SMILES: [2H]c1c([2H])c([2H])c(-c2nc3ccccc3n2-c2cccc(-c3c4ccccc4c(-c4ccccc4-c4ccccc4)c4ccccc34)c2)c([2H])c1[2H]]

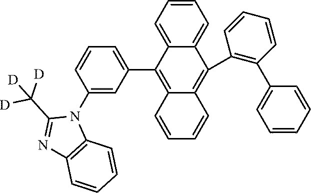 [SMILES: [2H]C([2H])([2H])c1nc2ccccc2n1-c1cccc(-c2c3ccccc3c(-c3ccccc3-c3ccccc3)c3ccccc23)c1]

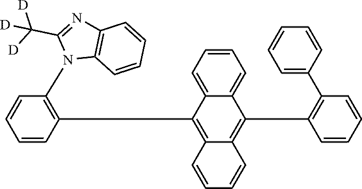 [SMILES: [2H]C([2H])([2H])c1nc2ccccc2n1-c1ccccc1-c1c2ccccc2c(-c2ccccc2-c2ccccc2)c2ccccc12]

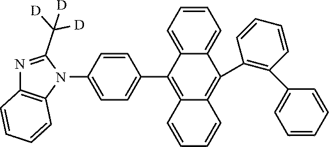 [SMILES: [2H]C([2H])([2H])c1nc2ccccc2n1-c1ccc(-c2c3ccccc3c(-c3ccccc3-c3ccccc3)c3ccccc23)cc1]

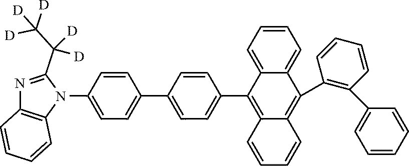 [SMILES: [2H]C([2H])([2H])C([2H])([2H])c1nc2ccccc2n1-c1ccc(-c2ccc(-c3c4ccccc4c(-c4ccccc4-c4ccccc4)c4ccccc34)cc2)cc1]

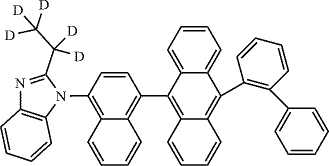 [SMILES: [2H]C([2H])([2H])C([2H])([2H])c1nc2ccccc2n1-c1ccc(-c2c3ccccc3c(-c3ccccc3-c3ccccc3)c3ccccc23)c2ccccc12]

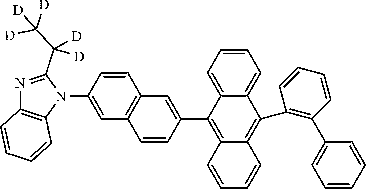 [SMILES: [2H]C([2H])([2H])C([2H])([2H])c1nc2ccccc2n1-c1ccc2cc(-c3c4ccccc4c(-c4ccccc4-c4ccccc4)c4ccccc34)ccc2c1]

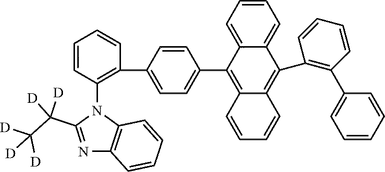 [SMILES: [2H]C([2H])([2H])C([2H])([2H])c1nc2ccccc2n1-c1ccccc1-c1ccc(-c2c3ccccc3c(-c3ccccc3-c3ccccc3)c3ccccc23)cc1]

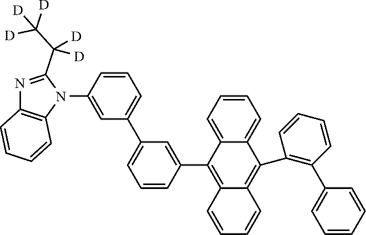 [SMILES: [2H]C([2H])([2H])C([2H])([2H])c1nc2ccccc2n1-c1cccc(-c2cccc(-c3c4ccccc4c(-c4ccccc4-c4ccccc4)c4ccccc34)c2)c1]

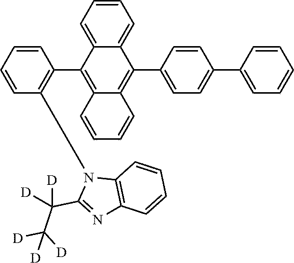 [SMILES: [2H]C([2H])([2H])C([2H])([2H])c1nc2ccccc2n1-c1ccccc1-c1c2ccccc2c(-c2ccc(-c3ccccc3)cc2)c2ccccc12]

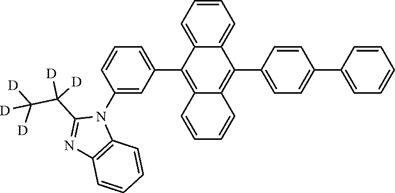 [SMILES: [2H]C([2H])([2H])C([2H])([2H])c1nc2ccccc2n1-c1cccc(-c2c3ccccc3c(-c3ccc(-c4ccccc4)cc3)c3ccccc23)c1]

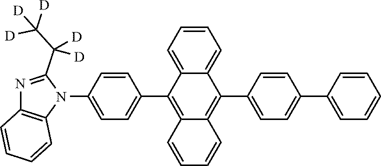 [SMILES: [2H]C([2H])([2H])C([2H])([2H])c1nc2ccccc2n1-c1ccc(-c2c3ccccc3c(-c3ccc(-c4ccccc4)cc3)c3ccccc23)cc1]

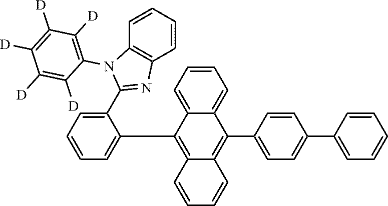 [SMILES: [2H]c1c([2H])c([2H])c(-n2c(-c3ccccc3-c3c4ccccc4c(-c4ccc(-c5ccccc5)cc4)c4ccccc34)nc3ccccc32)c([2H])c1[2H]]

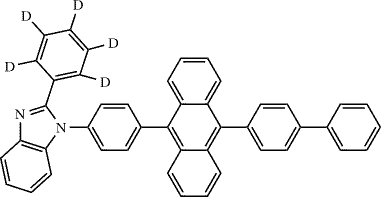 [SMILES: [2H]c1c([2H])c([2H])c(-c2nc3ccccc3n2-c2ccc(-c3c4ccccc4c(-c4ccc(-c5ccccc5)cc4)c4ccccc34)cc2)c([2H])c1[2H]]

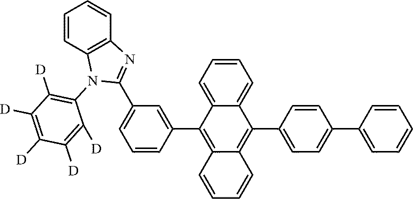 [SMILES: [2H]c1c([2H])c([2H])c(-n2c(-c3cccc(-c4c5ccccc5c(-c5ccc(-c6ccccc6)cc5)c5ccccc45)c3)nc3ccccc32)c([2H])c1[2H]]

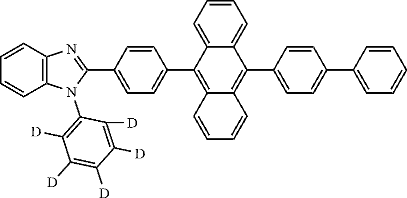 [SMILES: [2H]c1c([2H])c([2H])c(-n2c(-c3ccc(-c4c5ccccc5c(-c5ccc(-c6ccccc6)cc5)c5ccccc45)cc3)nc3ccccc32)c([2H])c1[2H]]

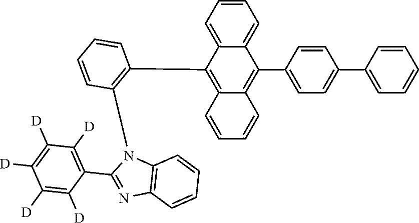 [SMILES: [2H]c1c([2H])c([2H])c(-c2nc3ccccc3n2-c2ccccc2-c2c3ccccc3c(-c3ccc(-c4ccccc4)cc3)c3ccccc23)c([2H])c1[2H]]

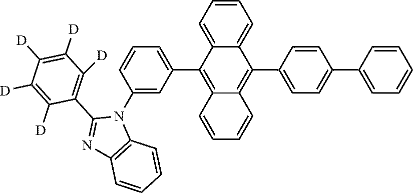 [SMILES: [2H]c1c([2H])c([2H])c(-c2nc3ccccc3n2-c2cccc(-c3c4ccccc4c(-c4ccc(-c5ccccc5)cc4)c4ccccc34)c2)c([2H])c1[2H]]

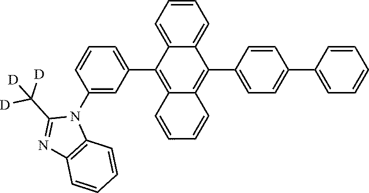 [SMILES: [2H]C([2H])([2H])c1nc2ccccc2n1-c1cccc(-c2c3ccccc3c(-c3ccc(-c4ccccc4)cc3)c3ccccc23)c1]

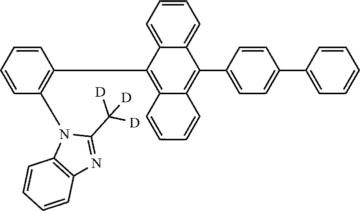 [SMILES: [2H]C([2H])([2H])c1nc2ccccc2n1-c1ccccc1-c1c2ccccc2c(-c2ccc(-c3ccccc3)cc2)c2ccccc12]

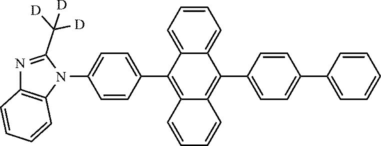 [SMILES: [2H]C([2H])([2H])c1nc2ccccc2n1-c1ccc(-c2c3ccccc3c(-c3ccc(-c4ccccc4)cc3)c3ccccc23)cc1]

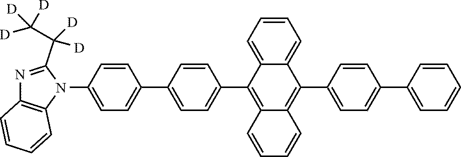 [SMILES: [2H]C([2H])([2H])C([2H])([2H])c1nc2ccccc2n1-c1ccc(-c2ccc(-c3c4ccccc4c(-c4ccc(-c5ccccc5)cc4)c4ccccc34)cc2)cc1]

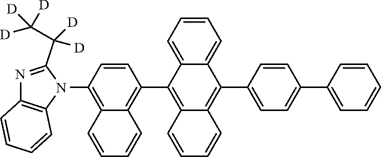 [SMILES: [2H]C([2H])([2H])C([2H])([2H])c1nc2ccccc2n1-c1ccc(-c2c3ccccc3c(-c3ccc(-c4ccccc4)cc3)c3ccccc23)c2ccccc12]

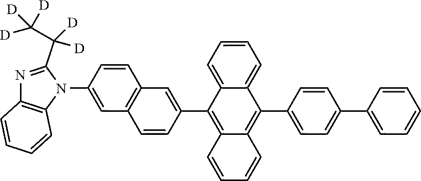 [SMILES: [2H]C([2H])([2H])C([2H])([2H])c1nc2ccccc2n1-c1ccc2cc(-c3c4ccccc4c(-c4ccc(-c5ccccc5)cc4)c4ccccc34)ccc2c1]

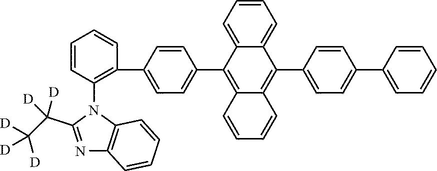 [SMILES: [2H]C([2H])([2H])C([2H])([2H])c1nc2ccccc2n1-c1ccccc1-c1ccc(-c2c3ccccc3c(-c3ccc(-c4ccccc4)cc3)c3ccccc23)cc1]

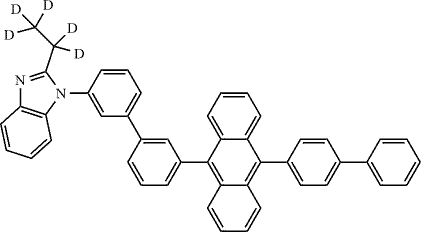 [SMILES: [2H]C([2H])([2H])C([2H])([2H])c1nc2ccccc2n1-c1cccc(-c2cccc(-c3c4ccccc4c(-c4ccc(-c5ccccc5)cc4)c4ccccc34)c2)c1]

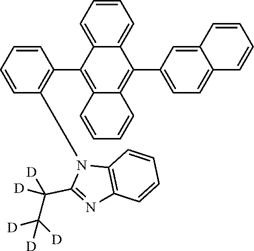 [SMILES: [2H]C([2H])([2H])C([2H])([2H])c1nc2ccccc2n1-c1ccccc1-c1c2ccccc2c(-c2ccc3ccccc3c2)c2ccccc12]

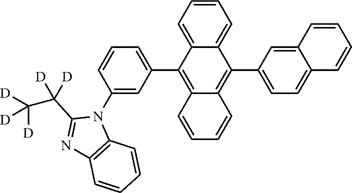 [SMILES: [2H]C([2H])([2H])C([2H])([2H])c1nc2ccccc2n1-c1cccc(-c2c3ccccc3c(-c3ccc4ccccc4c3)c3ccccc23)c1]

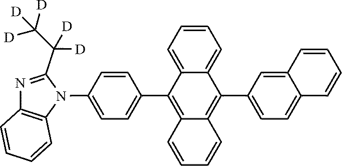 [SMILES: [2H]C([2H])([2H])C([2H])([2H])c1nc2ccccc2n1-c1ccc(-c2c3ccccc3c(-c3ccc4ccccc4c3)c3ccccc23)cc1]

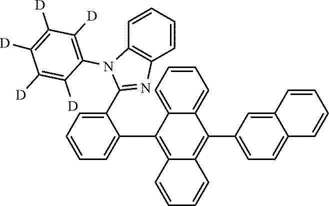 [SMILES: [2H]c1c([2H])c([2H])c(-n2c(-c3ccccc3-c3c4ccccc4c(-c4ccc5ccccc5c4)c4ccccc34)nc3ccccc32)c([2H])c1[2H]]

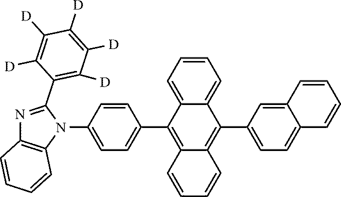 [SMILES: [2H]c1c([2H])c([2H])c(-c2nc3ccccc3n2-c2ccc(-c3c4ccccc4c(-c4ccc5ccccc5c4)c4ccccc34)cc2)c([2H])c1[2H]]

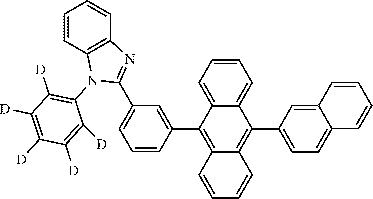 [SMILES: [2H]c1c([2H])c([2H])c(-n2c(-c3cccc(-c4c5ccccc5c(-c5ccc6ccccc6c5)c5ccccc45)c3)nc3ccccc32)c([2H])c1[2H]]

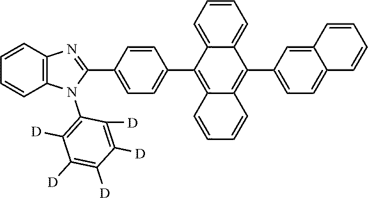 [SMILES: [2H]c1c([2H])c([2H])c(-n2c(-c3ccc(-c4c5ccccc5c(-c5ccc6ccccc6c5)c5ccccc45)cc3)nc3ccccc32)c([2H])c1[2H]]

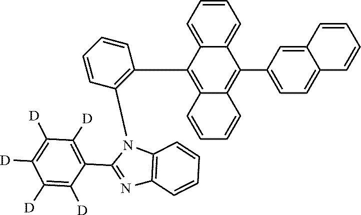 [SMILES: [2H]c1c([2H])c([2H])c(-c2nc3ccccc3n2-c2ccccc2-c2c3ccccc3c(-c3ccc4ccccc4c3)c3ccccc23)c([2H])c1[2H]]

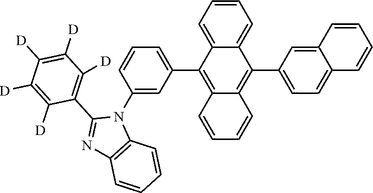 [SMILES: [2H]c1c([2H])c([2H])c(-c2nc3ccccc3n2-c2cccc(-c3c4ccccc4c(-c4ccc5ccccc5c4)c4ccccc34)c2)c([2H])c1[2H]]

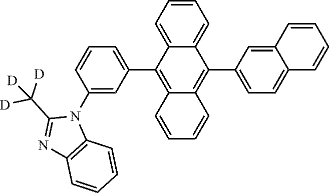 [SMILES: [2H]C([2H])([2H])c1nc2ccccc2n1-c1cccc(-c2c3ccccc3c(-c3ccc4ccccc4c3)c3ccccc23)c1]

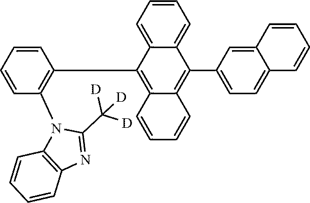 [SMILES: [2H]C([2H])([2H])c1nc2ccccc2n1-c1ccccc1-c1c2ccccc2c(-c2ccc3ccccc3c2)c2ccccc12]

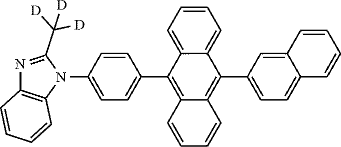 [SMILES: [2H]C([2H])([2H])c1nc2ccccc2n1-c1ccc(-c2c3ccccc3c(-c3ccc4ccccc4c3)c3ccccc23)cc1]

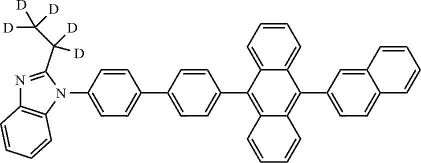 [SMILES: [2H]C([2H])([2H])C([2H])([2H])c1nc2ccccc2n1-c1ccc(-c2ccc(-c3c4ccccc4c(-c4ccc5ccccc5c4)c4ccccc34)cc2)cc1]

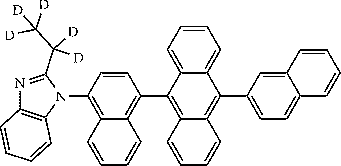 [SMILES: [2H]C([2H])([2H])C([2H])([2H])c1nc2ccccc2n1-c1ccc(-c2c3ccccc3c(-c3ccc4ccccc4c3)c3ccccc23)c2ccccc12]

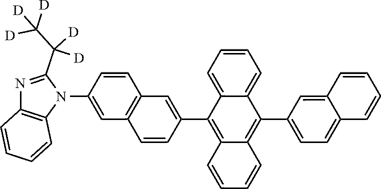 [SMILES: [2H]C([2H])([2H])C([2H])([2H])c1nc2ccccc2n1-c1ccc2cc(-c3c4ccccc4c(-c4ccc5ccccc5c4)c4ccccc34)ccc2c1]

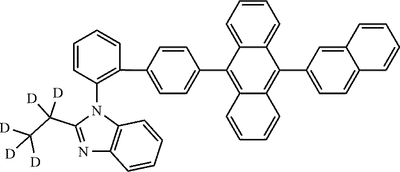 [SMILES: [2H]C([2H])([2H])C([2H])([2H])c1nc2ccccc2n1-c1ccccc1-c1ccc(-c2c3ccccc3c(-c3ccc4ccccc4c3)c3ccccc23)cc1]

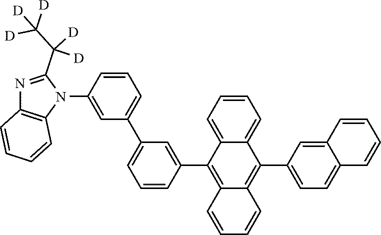 [SMILES: [2H]C([2H])([2H])C([2H])([2H])c1nc2ccccc2n1-c1cccc(-c2cccc(-c3c4ccccc4c(-c4ccc5ccccc5c4)c4ccccc34)c2)c1]

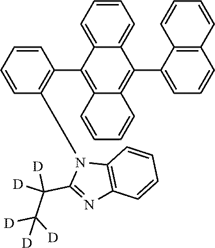 [SMILES: [2H]C([2H])([2H])C([2H])([2H])c1nc2ccccc2n1-c1ccccc1-c1c2ccccc2c(-c2cccc3ccccc23)c2ccccc12]

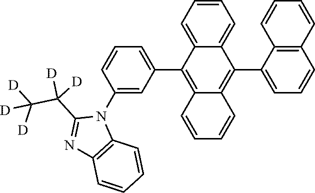 [SMILES: [2H]C([2H])([2H])C([2H])([2H])c1nc2ccccc2n1-c1cccc(-c2c3ccccc3c(-c3cccc4ccccc34)c3ccccc23)c1]

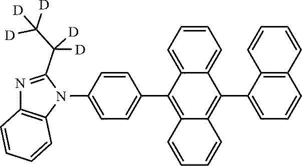 [SMILES: [2H]C([2H])([2H])C([2H])([2H])c1nc2ccccc2n1-c1ccc(-c2c3ccccc3c(-c3cccc4ccccc34)c3ccccc23)cc1]

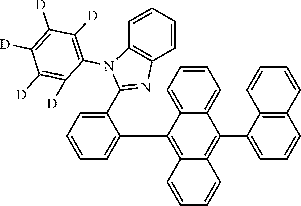 [SMILES: [2H]c1c([2H])c([2H])c(-n2c(-c3ccccc3-c3c4ccccc4c(-c4cccc5ccccc45)c4ccccc34)nc3ccccc32)c([2H])c1[2H]]

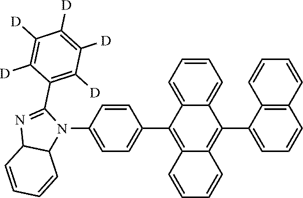 [SMILES: [2H]c1c([2H])c([2H])c(C2=NC3C=CC=CC3N2c2ccc(-c3c4ccccc4c(-c4cccc5ccccc45)c4ccccc34)cc2)c([2H])c1[2H]]

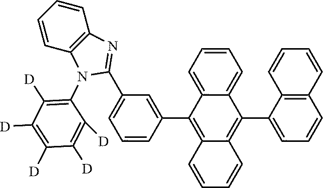 [SMILES: [2H]c1c([2H])c([2H])c(-n2c(-c3cccc(-c4c5ccccc5c(-c5cccc6ccccc56)c5ccccc45)c3)nc3ccccc32)c([2H])c1[2H]]

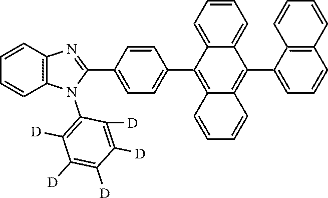 [SMILES: [2H]c1c([2H])c([2H])c(-n2c(-c3ccc(-c4c5ccccc5c(-c5cccc6ccccc56)c5ccccc45)cc3)nc3ccccc32)c([2H])c1[2H]]

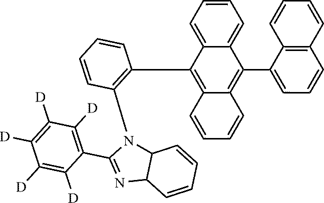 [SMILES: [2H]c1c([2H])c([2H])c(C2=NC3C=CC=CC3N2c2ccccc2-c2c3ccccc3c(-c3cccc4ccccc34)c3ccccc23)c([2H])c1[2H]]

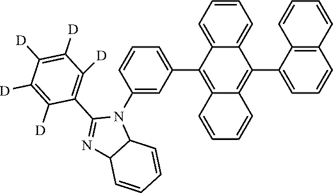 [SMILES: [2H]c1c([2H])c([2H])c(C2=NC3C=CC=CC3N2c2cccc(-c3c4ccccc4c(-c4cccc5ccccc45)c4ccccc34)c2)c([2H])c1[2H]]

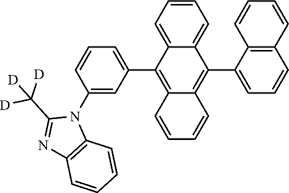 [SMILES: [2H]C([2H])([2H])c1nc2ccccc2n1-c1cccc(-c2c3ccccc3c(-c3cccc4ccccc34)c3ccccc23)c1]

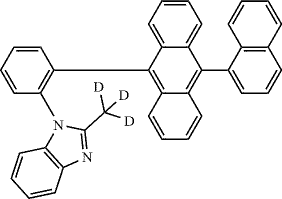 [SMILES: [2H]C([2H])([2H])c1nc2ccccc2n1-c1ccccc1-c1c2ccccc2c(-c2cccc3ccccc23)c2ccccc12]

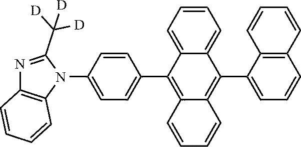 [SMILES: [2H]C([2H])([2H])c1nc2ccccc2n1-c1ccc(-c2c3ccccc3c(-c3cccc4ccccc34)c3ccccc23)cc1]

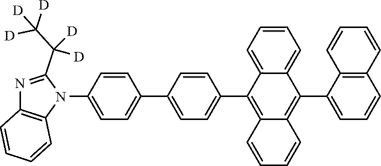 [SMILES: [2H]C([2H])([2H])C([2H])([2H])c1nc2ccccc2n1-c1ccc(-c2ccc(-c3c4ccccc4c(-c4cccc5ccccc45)c4ccccc34)cc2)cc1]

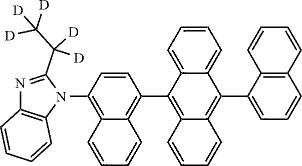 [SMILES: [2H]C([2H])([2H])C([2H])([2H])c1nc2ccccc2n1-c1ccc(-c2c3ccccc3c(-c3cccc4ccccc34)c3ccccc23)c2ccccc12]

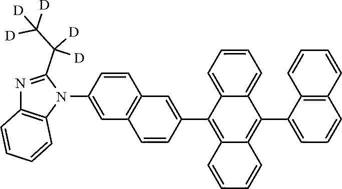 [SMILES: [2H]C([2H])([2H])C([2H])([2H])c1nc2ccccc2n1-c1ccc2cc(-c3c4ccccc4c(-c4cccc5ccccc45)c4ccccc34)ccc2c1]

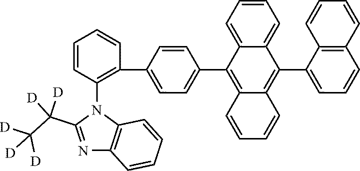 [SMILES: [2H]C([2H])([2H])C([2H])([2H])c1nc2ccccc2n1-c1ccccc1-c1ccc(-c2c3ccccc3c(-c3cccc4ccccc34)c3ccccc23)cc1]

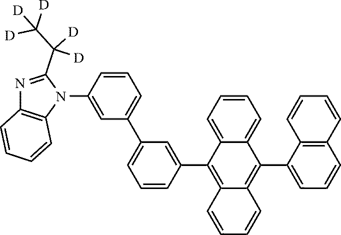 [SMILES: [2H]C([2H])([2H])C([2H])([2H])c1nc2ccccc2n1-c1cccc(-c2cccc(-c3c4ccccc4c(-c4cccc5ccccc45)c4ccccc34)c2)c1]

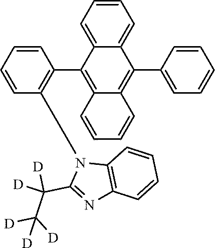 [SMILES: [2H]C([2H])([2H])C([2H])([2H])c1nc2ccccc2n1-c1ccccc1-c1c2ccccc2c(-c2ccccc2)c2ccccc12]

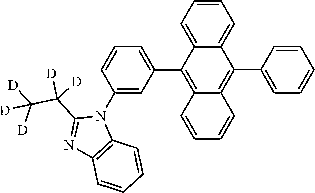 [SMILES: [2H]C([2H])([2H])C([2H])([2H])c1nc2ccccc2n1-c1cccc(-c2c3ccccc3c(-c3ccccc3)c3ccccc23)c1]

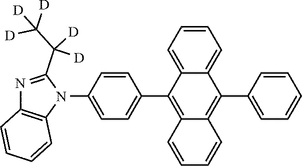 [SMILES: [2H]C([2H])([2H])C([2H])([2H])c1nc2ccccc2n1-c1ccc(-c2c3ccccc3c(-c3ccccc3)c3ccccc23)cc1]

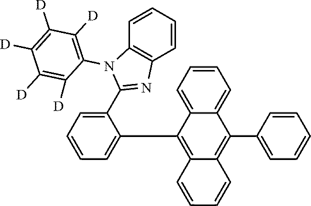 [SMILES: [2H]c1c([2H])c([2H])c(-n2c(-c3ccccc3-c3c4ccccc4c(-c4ccccc4)c4ccccc34)nc3ccccc32)c([2H])c1[2H]]

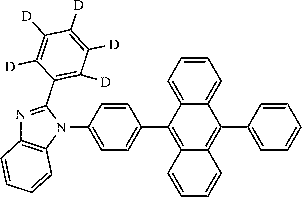 [SMILES: [2H]c1c([2H])c([2H])c(-c2nc3ccccc3n2-c2ccc(-c3c4ccccc4c(-c4ccccc4)c4ccccc34)cc2)c([2H])c1[2H]]

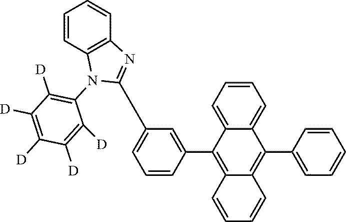 [SMILES: [2H]c1c([2H])c([2H])c(-n2c(-c3cccc(-c4c5ccccc5c(-c5ccccc5)c5ccccc45)c3)nc3ccccc32)c([2H])c1[2H]]

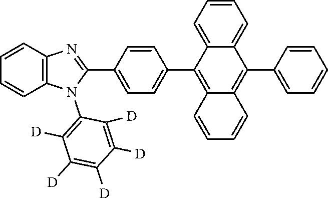 [SMILES: [2H]c1c([2H])c([2H])c(-n2c(-c3ccc(-c4c5ccccc5c(-c5ccccc5)c5ccccc45)cc3)nc3ccccc32)c([2H])c1[2H]]

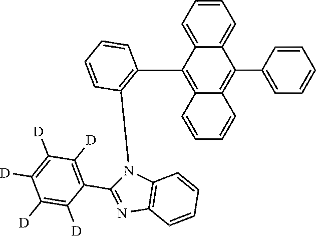 [SMILES: [2H]c1c([2H])c([2H])c(-c2nc3ccccc3n2-c2ccccc2-c2c3ccccc3c(-c3ccccc3)c3ccccc23)c([2H])c1[2H]]

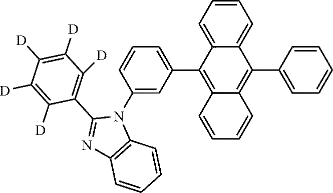 [SMILES: [2H]c1c([2H])c([2H])c(-c2nc3ccccc3n2-c2cccc(-c3c4ccccc4c(-c4ccccc4)c4ccccc34)c2)c([2H])c1[2H]]

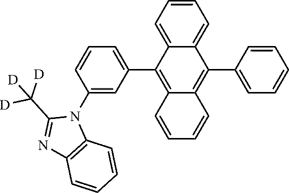 [SMILES: [2H]C([2H])([2H])c1nc2ccccc2n1-c1cccc(-c2c3ccccc3c(-c3ccccc3)c3ccccc23)c1]

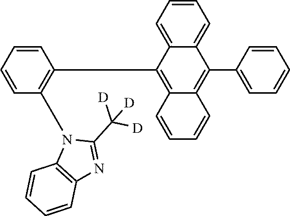 [SMILES: [2H]C([2H])([2H])c1nc2ccccc2n1-c1ccccc1-c1c2ccccc2c(-c2ccccc2)c2ccccc12]

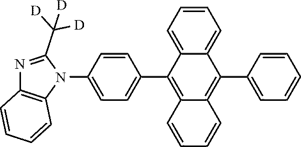 [SMILES: [2H]C([2H])([2H])c1nc2ccccc2n1-c1ccc(-c2c3ccccc3c(-c3ccccc3)c3ccccc23)cc1]

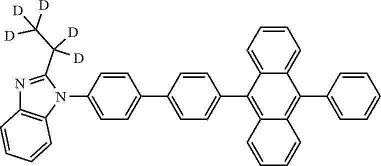 [SMILES: [2H]C([2H])([2H])C([2H])([2H])c1nc2ccccc2n1-c1ccc(-c2ccc(-c3c4ccccc4c(-c4ccccc4)c4ccccc34)cc2)cc1]

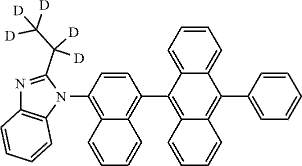 [SMILES: [2H]C([2H])([2H])C([2H])([2H])c1nc2ccccc2n1-c1ccc(-c2c3ccccc3c(-c3ccccc3)c3ccccc23)c2ccccc12]

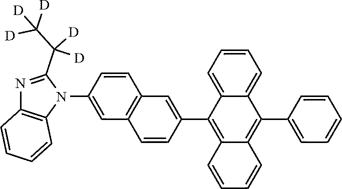 [SMILES: [2H]C([2H])([2H])C([2H])([2H])c1nc2ccccc2n1-c1ccc2cc(-c3c4ccccc4c(-c4ccccc4)c4ccccc34)ccc2c1]

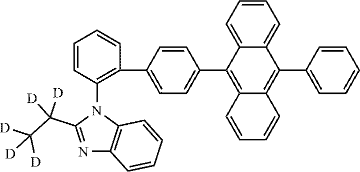 [SMILES: [2H]C([2H])([2H])C([2H])([2H])c1nc2ccccc2n1-c1ccccc1-c1ccc(-c2c3ccccc3c(-c3ccccc3)c3ccccc23)cc1]

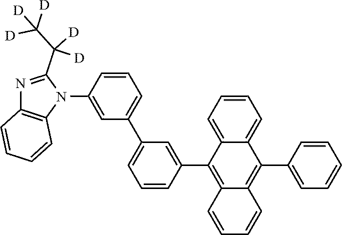 [SMILES: [2H]C([2H])([2H])C([2H])([2H])c1nc2ccccc2n1-c1cccc(-c2cccc(-c3c4ccccc4c(-c4ccccc4)c4ccccc34)c2)c1]

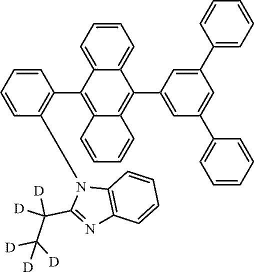 [SMILES: [2H]C([2H])([2H])C([2H])([2H])c1nc2ccccc2n1-c1ccccc1-c1c2ccccc2c(-c2cc(-c3ccccc3)cc(-c3ccccc3)c2)c2ccccc12]

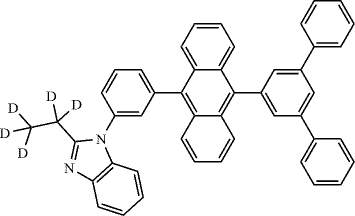 [SMILES: [2H]C([2H])([2H])C([2H])([2H])c1nc2ccccc2n1-c1cccc(-c2c3ccccc3c(-c3cc(-c4ccccc4)cc(-c4ccccc4)c3)c3ccccc23)c1]

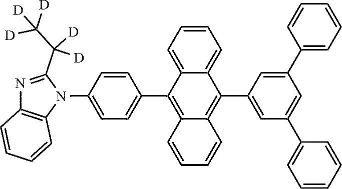 [SMILES: [2H]C([2H])([2H])C([2H])([2H])c1nc2ccccc2n1-c1ccc(-c2c3ccccc3c(-c3cc(-c4ccccc4)cc(-c4ccccc4)c3)c3ccccc23)cc1]

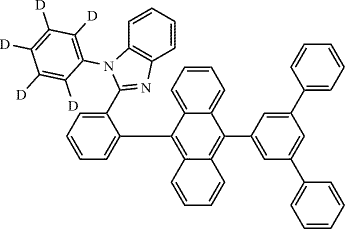 [SMILES: [2H]c1c([2H])c([2H])c(-n2c(-c3ccccc3-c3c4ccccc4c(-c4cc(-c5ccccc5)cc(-c5ccccc5)c4)c4ccccc34)nc3ccccc32)c([2H])c1[2H]]

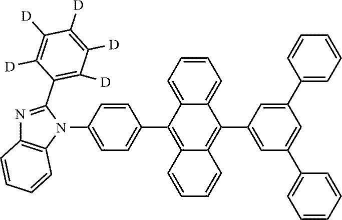 [SMILES: [2H]c1c([2H])c([2H])c(-c2nc3ccccc3n2-c2ccc(-c3c4ccccc4c(-c4cc(-c5ccccc5)cc(-c5ccccc5)c4)c4ccccc34)cc2)c([2H])c1[2H]]

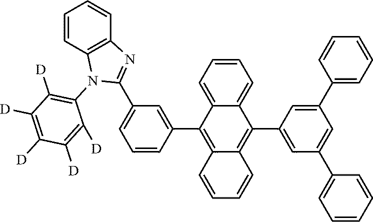 [SMILES: [2H]c1c([2H])c([2H])c(-n2c(-c3cccc(-c4c5ccccc5c(-c5cc(-c6ccccc6)cc(-c6ccccc6)c5)c5ccccc45)c3)nc3ccccc32)c([2H])c1[2H]]

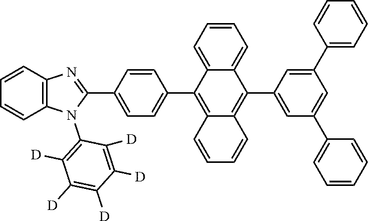 [SMILES: [2H]c1c([2H])c([2H])c(-n2c(-c3ccc(-c4c5ccccc5c(-c5cc(-c6ccccc6)cc(-c6ccccc6)c5)c5ccccc45)cc3)nc3ccccc32)c([2H])c1[2H]]

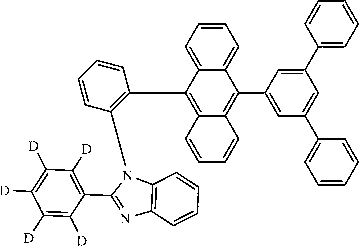 [SMILES: [2H]c1c([2H])c([2H])c(-c2nc3ccccc3n2-c2ccccc2-c2c3ccccc3c(-c3cc(-c4ccccc4)cc(-c4ccccc4)c3)c3ccccc23)c([2H])c1[2H]]

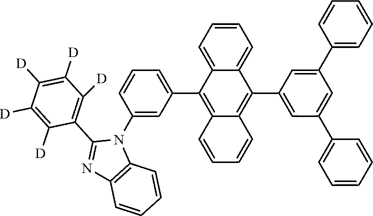 [SMILES: [2H]c1c([2H])c([2H])c(-c2nc3ccccc3n2-c2cccc(-c3c4ccccc4c(-c4cc(-c5ccccc5)cc(-c5ccccc5)c4)c4ccccc34)c2)c([2H])c1[2H]]

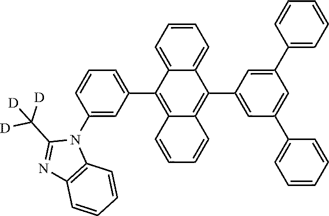 [SMILES: [2H]C([2H])([2H])c1nc2ccccc2n1-c1cccc(-c2c3ccccc3c(-c3cc(-c4ccccc4)cc(-c4ccccc4)c3)c3ccccc23)c1]

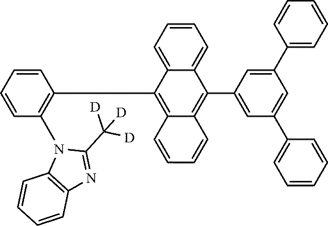 [SMILES: [2H]C([2H])([2H])c1nc2ccccc2n1-c1ccccc1-c1c2ccccc2c(-c2cc(-c3ccccc3)cc(-c3ccccc3)c2)c2ccccc12]

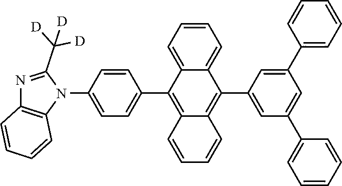 [SMILES: [2H]C([2H])([2H])c1nc2ccccc2n1-c1ccc(-c2c3ccccc3c(-c3cc(-c4ccccc4)cc(-c4ccccc4)c3)c3ccccc23)cc1]

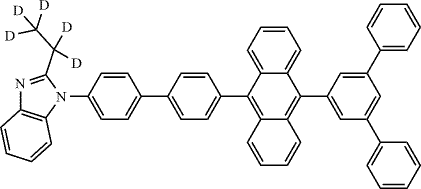 [SMILES: [2H]C([2H])([2H])C([2H])([2H])c1nc2ccccc2n1-c1ccc(-c2ccc(-c3c4ccccc4c(-c4cc(-c5ccccc5)cc(-c5ccccc5)c4)c4ccccc34)cc2)cc1]

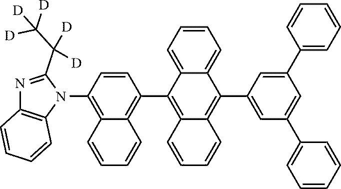 [SMILES: [2H]C([2H])([2H])C([2H])([2H])c1nc2ccccc2n1-c1ccc(-c2c3ccccc3c(-c3cc(-c4ccccc4)cc(-c4ccccc4)c3)c3ccccc23)c2ccccc12]

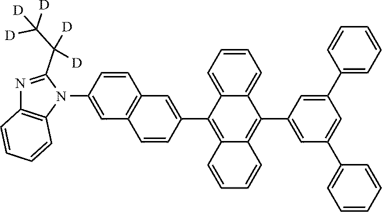 [SMILES: [2H]C([2H])([2H])C([2H])([2H])c1nc2ccccc2n1-c1ccc2cc(-c3c4ccccc4c(-c4cc(-c5ccccc5)cc(-c5ccccc5)c4)c4ccccc34)ccc2c1]

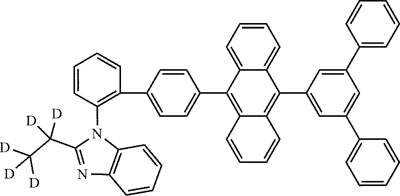 [SMILES: [2H]C([2H])([2H])C([2H])([2H])c1nc2ccccc2n1-c1ccccc1-c1ccc(-c2c3ccccc3c(-c3cc(-c4ccccc4)cc(-c4ccccc4)c3)c3ccccc23)cc1]

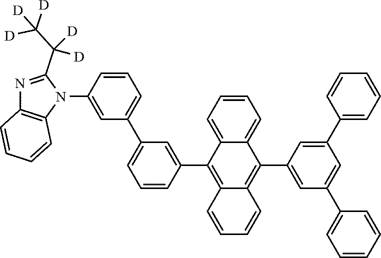 [SMILES: [2H]C([2H])([2H])C([2H])([2H])c1nc2ccccc2n1-c1cccc(-c2cccc(-c3c4ccccc4c(-c4cc(-c5ccccc5)cc(-c5ccccc5)c4)c4ccccc34)c2)c1]

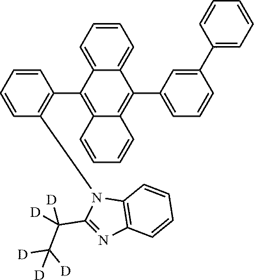 [SMILES: [2H]C([2H])([2H])C([2H])([2H])c1nc2ccccc2n1-c1ccccc1-c1c2ccccc2c(-c2cccc(-c3ccccc3)c2)c2ccccc12]

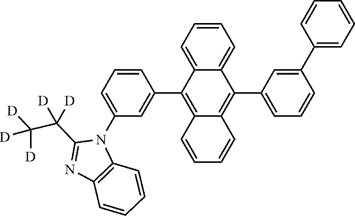 [SMILES: [2H]C([2H])([2H])C([2H])([2H])c1nc2ccccc2n1-c1cccc(-c2c3ccccc3c(-c3cccc(-c4ccccc4)c3)c3ccccc23)c1]

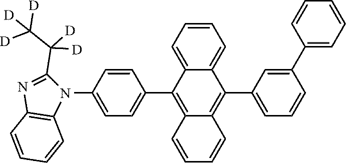 [SMILES: [2H]C([2H])([2H])C([2H])([2H])c1nc2ccccc2n1-c1ccc(-c2c3ccccc3c(-c3cccc(-c4ccccc4)c3)c3ccccc23)cc1]

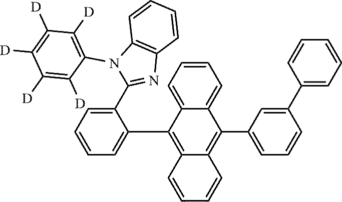 [SMILES: [2H]c1c([2H])c([2H])c(-n2c(-c3ccccc3-c3c4ccccc4c(-c4cccc(-c5ccccc5)c4)c4ccccc34)nc3ccccc32)c([2H])c1[2H]]

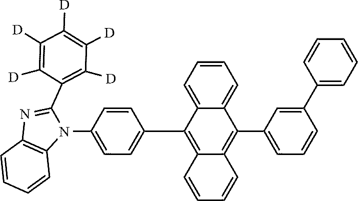 [SMILES: [2H]c1c([2H])c([2H])c(-c2nc3ccccc3n2-c2ccc(-c3c4ccccc4c(-c4cccc(-c5ccccc5)c4)c4ccccc34)cc2)c([2H])c1[2H]]

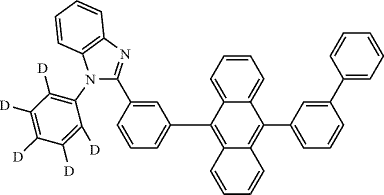 [SMILES: [2H]c1c([2H])c([2H])c(-n2c(-c3cccc(-c4c5ccccc5c(-c5cccc(-c6ccccc6)c5)c5ccccc45)c3)nc3ccccc32)c([2H])c1[2H]]

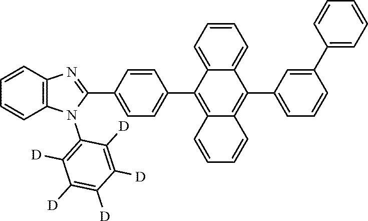 [SMILES: [2H]c1c([2H])c([2H])c(-n2c(-c3ccc(-c4c5ccccc5c(-c5cccc(-c6ccccc6)c5)c5ccccc45)cc3)nc3ccccc32)c([2H])c1[2H]]

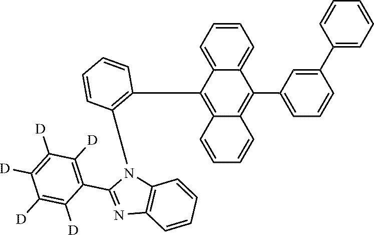 [SMILES: [2H]c1c([2H])c([2H])c(-c2nc3ccccc3n2-c2ccccc2-c2c3ccccc3c(-c3cccc(-c4ccccc4)c3)c3ccccc23)c([2H])c1[2H]]

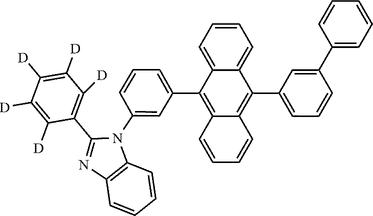 [SMILES: [2H]c1c([2H])c([2H])c(-c2nc3ccccc3n2-c2cccc(-c3c4ccccc4c(-c4cccc(-c5ccccc5)c4)c4ccccc34)c2)c([2H])c1[2H]]

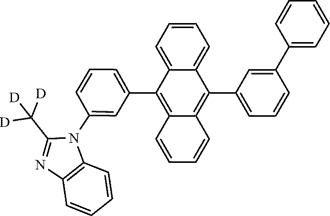 [SMILES: [2H]C([2H])([2H])c1nc2ccccc2n1-c1cccc(-c2c3ccccc3c(-c3cccc(-c4ccccc4)c3)c3ccccc23)c1]

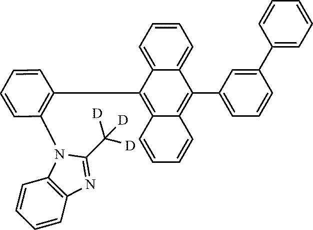 [SMILES: [2H]C([2H])([2H])c1nc2ccccc2n1-c1ccccc1-c1c2ccccc2c(-c2cccc(-c3ccccc3)c2)c2ccccc12]

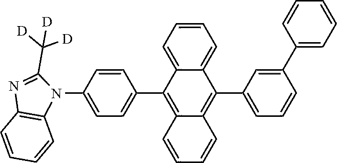 [SMILES: [2H]C([2H])([2H])c1nc2ccccc2n1-c1ccc(-c2c3ccccc3c(-c3cccc(-c4ccccc4)c3)c3ccccc23)cc1]

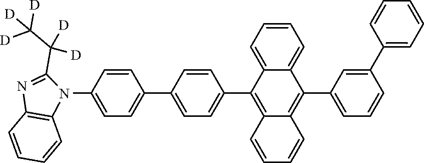 [SMILES: [2H]C([2H])([2H])C([2H])([2H])c1nc2ccccc2n1-c1ccc(-c2ccc(-c3c4ccccc4c(-c4cccc(-c5ccccc5)c4)c4ccccc34)cc2)cc1]

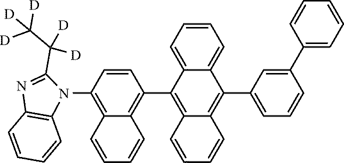 [SMILES: [2H]C([2H])([2H])C([2H])([2H])c1nc2ccccc2n1-c1ccc(-c2c3ccccc3c(-c3cccc(-c4ccccc4)c3)c3ccccc23)c2ccccc12]

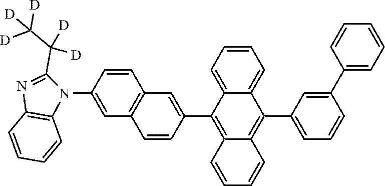 [SMILES: [2H]C([2H])([2H])C([2H])([2H])c1nc2ccccc2n1-c1ccc2cc(-c3c4ccccc4c(-c4cccc(-c5ccccc5)c4)c4ccccc34)ccc2c1]

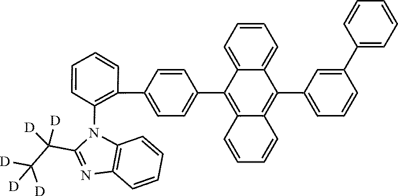 [SMILES: [2H]C([2H])([2H])C([2H])([2H])c1nc2ccccc2n1-c1ccccc1-c1ccc(-c2c3ccccc3c(-c3cccc(-c4ccccc4)c3)c3ccccc23)cc1]

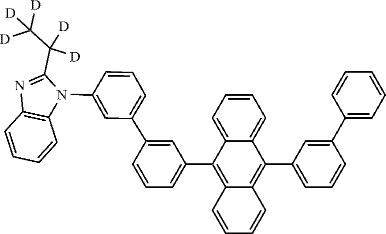 [SMILES: [2H]C([2H])([2H])C([2H])([2H])c1nc2ccccc2n1-c1cccc(-c2cccc(-c3c4ccccc4c(-c4cccc(-c5ccccc5)c4)c4ccccc34)c2)c1]

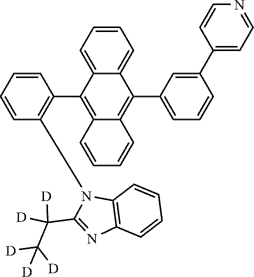 [SMILES: [2H]C([2H])([2H])C([2H])([2H])c1nc2ccccc2n1-c1ccccc1-c1c2ccccc2c(-c2cccc(-c3ccncc3)c2)c2ccccc12]

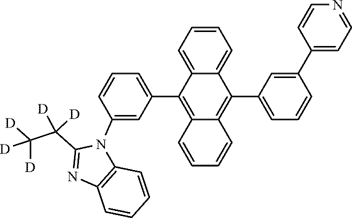 [SMILES: [2H]C([2H])([2H])C([2H])([2H])c1nc2ccccc2n1-c1cccc(-c2c3ccccc3c(-c3cccc(-c4ccncc4)c3)c3ccccc23)c1]

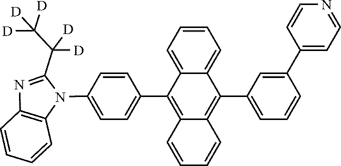 [SMILES: [2H]C([2H])([2H])C([2H])([2H])c1nc2ccccc2n1-c1ccc(-c2c3ccccc3c(-c3cccc(-c4ccncc4)c3)c3ccccc23)cc1]

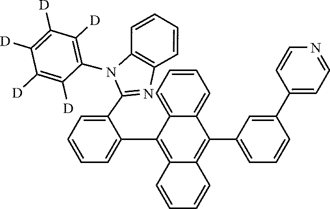 [SMILES: [2H]c1c([2H])c([2H])c(-n2c(-c3ccccc3-c3c4ccccc4c(-c4cccc(-c5ccncc5)c4)c4ccccc34)nc3ccccc32)c([2H])c1[2H]]

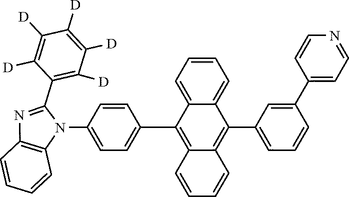 [SMILES: [2H]c1c([2H])c([2H])c(-c2nc3ccccc3n2-c2ccc(-c3c4ccccc4c(-c4cccc(-c5ccncc5)c4)c4ccccc34)cc2)c([2H])c1[2H]]

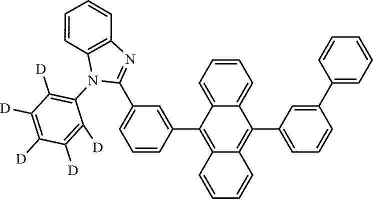 [SMILES: [2H]c1c([2H])c([2H])c(-n2c(-c3cccc(-c4c5ccccc5c(-c5cccc(-c6ccccc6)c5)c5ccccc45)c3)nc3ccccc32)c([2H])c1[2H]]

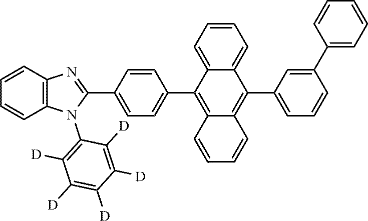 [SMILES: [2H]c1c([2H])c([2H])c(-n2c(-c3ccc(-c4c5ccccc5c(-c5cccc(-c6ccccc6)c5)c5ccccc45)cc3)nc3ccccc32)c([2H])c1[2H]]

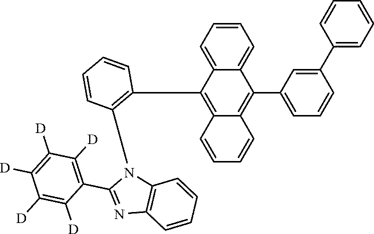 [SMILES: [2H]c1c([2H])c([2H])c(-c2nc3ccccc3n2-c2ccccc2-c2c3ccccc3c(-c3cccc(-c4ccccc4)c3)c3ccccc23)c([2H])c1[2H]]

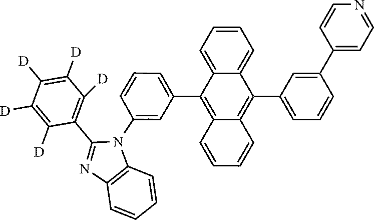 [SMILES: [2H]c1c([2H])c([2H])c(-c2nc3ccccc3n2-c2cccc(-c3c4ccccc4c(-c4cccc(-c5ccncc5)c4)c4ccccc34)c2)c([2H])c1[2H]]

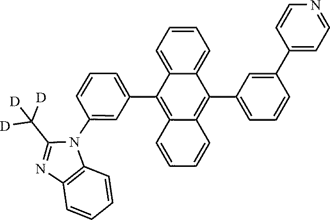 [SMILES: [2H]C([2H])([2H])c1nc2ccccc2n1-c1cccc(-c2c3ccccc3c(-c3cccc(-c4ccncc4)c3)c3ccccc23)c1]

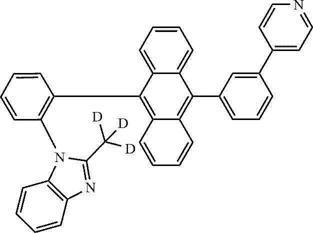 [SMILES: [2H]C([2H])([2H])c1nc2ccccc2n1-c1ccccc1-c1c2ccccc2c(-c2cccc(-c3ccncc3)c2)c2ccccc12]

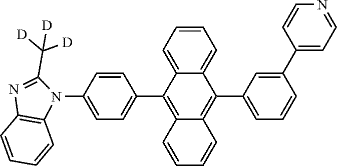 [SMILES: [2H]C([2H])([2H])c1nc2ccccc2n1-c1ccc(-c2c3ccccc3c(-c3cccc(-c4ccncc4)c3)c3ccccc23)cc1]

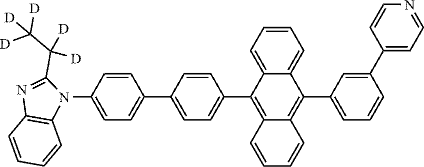 [SMILES: [2H]C([2H])([2H])C([2H])([2H])c1nc2ccccc2n1-c1ccc(-c2ccc(-c3c4ccccc4c(-c4cccc(-c5ccncc5)c4)c4ccccc34)cc2)cc1]

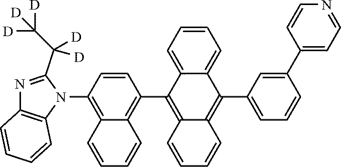 [SMILES: [2H]C([2H])([2H])C([2H])([2H])c1nc2ccccc2n1-c1ccc(-c2c3ccccc3c(-c3cccc(-c4ccncc4)c3)c3ccccc23)c2ccccc12]

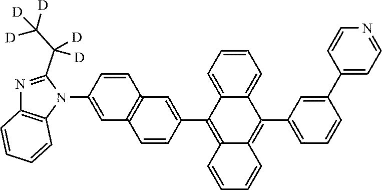 [SMILES: [2H]C([2H])([2H])C([2H])([2H])c1nc2ccccc2n1-c1ccc2cc(-c3c4ccccc4c(-c4cccc(-c5ccncc5)c4)c4ccccc34)ccc2c1]

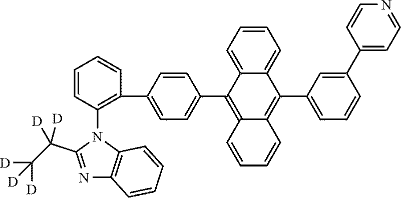 [SMILES: [2H]C([2H])([2H])C([2H])([2H])c1nc2ccccc2n1-c1ccccc1-c1ccc(-c2c3ccccc3c(-c3cccc(-c4ccncc4)c3)c3ccccc23)cc1]

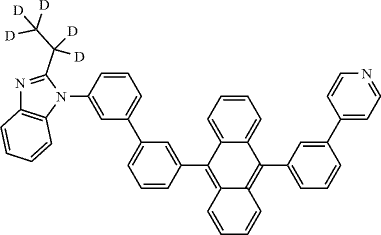 [SMILES: [2H]C([2H])([2H])C([2H])([2H])c1nc2ccccc2n1-c1cccc(-c2cccc(-c3c4ccccc4c(-c4cccc(-c5ccncc5)c4)c4ccccc34)c2)c1]

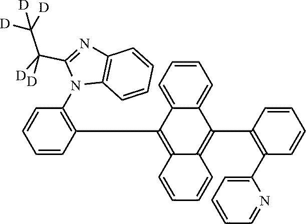 [SMILES: [2H]C([2H])([2H])C([2H])([2H])c1nc2ccccc2n1-c1ccccc1-c1c2ccccc2c(-c2ccccc2-c2ccccn2)c2ccccc12]

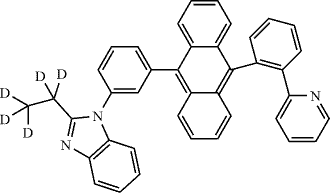 [SMILES: [2H]C([2H])([2H])C([2H])([2H])c1nc2ccccc2n1-c1cccc(-c2c3ccccc3c(-c3ccccc3-c3ccccn3)c3ccccc23)c1]

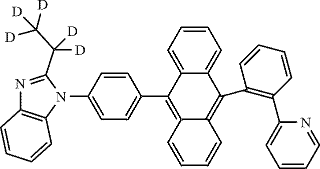 [SMILES: [2H]C([2H])([2H])C([2H])([2H])c1nc2ccccc2n1-c1ccc(-c2c3ccccc3c(-c3ccccc3-c3ccccn3)c3ccccc23)cc1]

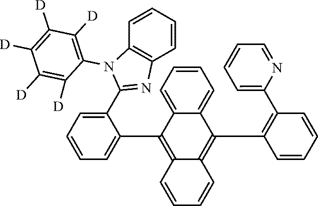 [SMILES: [2H]c1c([2H])c([2H])c(-n2c(-c3ccccc3-c3c4ccccc4c(-c4ccccc4-c4ccccn4)c4ccccc34)nc3ccccc32)c([2H])c1[2H]]

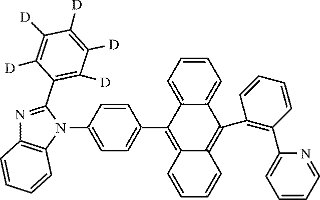 [SMILES: [2H]c1c([2H])c([2H])c(-c2nc3ccccc3n2-c2ccc(-c3c4ccccc4c(-c4ccccc4-c4ccccn4)c4ccccc34)cc2)c([2H])c1[2H]]

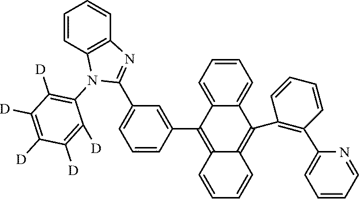 [SMILES: [2H]c1c([2H])c([2H])c(-n2c(-c3cccc(-c4c5ccccc5c(-c5ccccc5-c5ccccn5)c5ccccc45)c3)nc3ccccc32)c([2H])c1[2H]]

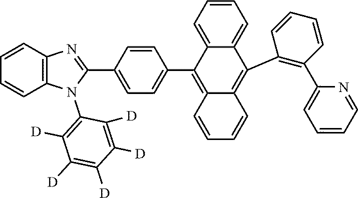 [SMILES: [2H]c1c([2H])c([2H])c(-n2c(-c3ccc(-c4c5ccccc5c(-c5ccccc5-c5ccccn5)c5ccccc45)cc3)nc3ccccc32)c([2H])c1[2H]]

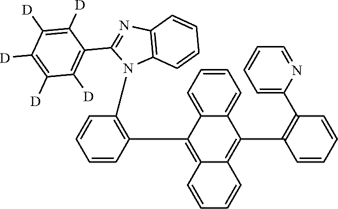 [SMILES: [2H]c1c([2H])c([2H])c(-c2nc3ccccc3n2-c2ccccc2-c2c3ccccc3c(-c3ccccc3-c3ccccn3)c3ccccc23)c([2H])c1[2H]]

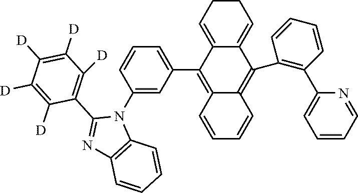 [SMILES: [2H]c1c([2H])c([2H])c(-c2nc3ccccc3n2-c2cccc(-c3c4c(c(-c5ccccc5-c5ccccn5)c5ccccc35)=CCCC=4)c2)c([2H])c1[2H]]

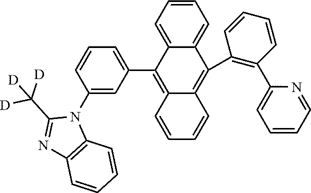 [SMILES: [2H]C([2H])([2H])c1nc2ccccc2n1-c1cccc(-c2c3ccccc3c(-c3ccccc3-c3ccccn3)c3ccccc23)c1]

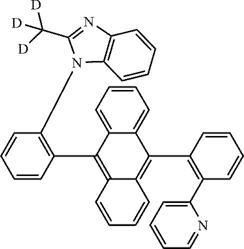 [SMILES: [2H]C([2H])([2H])c1nc2ccccc2n1-c1ccccc1-c1c2ccccc2c(-c2ccccc2-c2ccccn2)c2ccccc12]

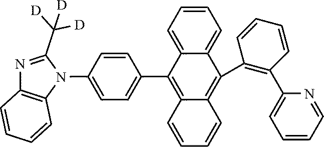 [SMILES: [2H]C([2H])([2H])c1nc2ccccc2n1-c1ccc(-c2c3ccccc3c(-c3ccccc3-c3ccccn3)c3ccccc23)cc1]

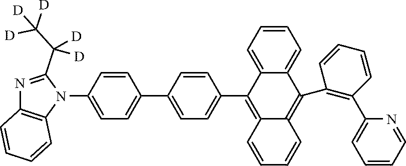 [SMILES: [2H]C([2H])([2H])C([2H])([2H])c1nc2ccccc2n1-c1ccc(-c2ccc(-c3c4ccccc4c(-c4ccccc4-c4ccccn4)c4ccccc34)cc2)cc1]

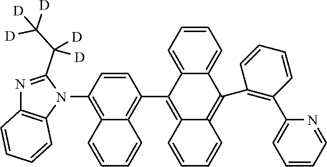 [SMILES: [2H]C([2H])([2H])C([2H])([2H])c1nc2ccccc2n1-c1ccc(-c2c3ccccc3c(-c3ccccc3-c3ccccn3)c3ccccc23)c2ccccc12]

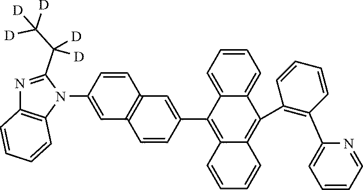 [SMILES: [2H]C([2H])([2H])C([2H])([2H])c1nc2ccccc2n1-c1ccc2cc(-c3c4ccccc4c(-c4ccccc4-c4ccccn4)c4ccccc34)ccc2c1]

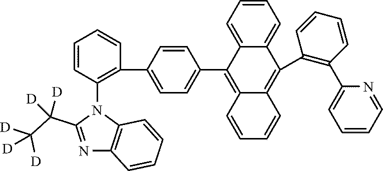 [SMILES: [2H]C([2H])([2H])C([2H])([2H])c1nc2ccccc2n1-c1ccccc1-c1ccc(-c2c3ccccc3c(-c3ccccc3-c3ccccn3)c3ccccc23)cc1]

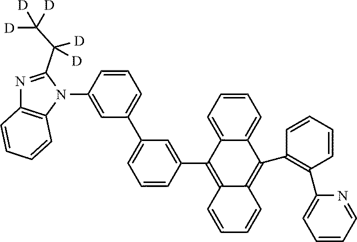 [SMILES: [2H]C([2H])([2H])C([2H])([2H])c1nc2ccccc2n1-c1cccc(-c2cccc(-c3c4ccccc4c(-c4ccccc4-c4ccccn4)c4ccccc34)c2)c1]

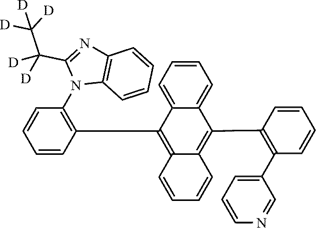 [SMILES: [2H]C([2H])([2H])C([2H])([2H])c1nc2ccccc2n1-c1ccccc1-c1c2ccccc2c(-c2ccccc2-c2cccnc2)c2ccccc12]

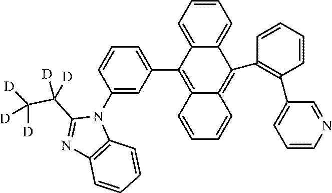 [SMILES: [2H]C([2H])([2H])C([2H])([2H])c1nc2ccccc2n1-c1cccc(-c2c3ccccc3c(-c3ccccc3-c3cccnc3)c3ccccc23)c1]

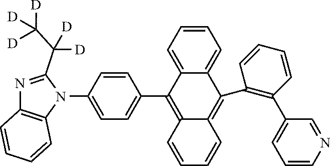 [SMILES: [2H]C([2H])([2H])C([2H])([2H])c1nc2ccccc2n1-c1ccc(-c2c3ccccc3c(-c3ccccc3-c3cccnc3)c3ccccc23)cc1]

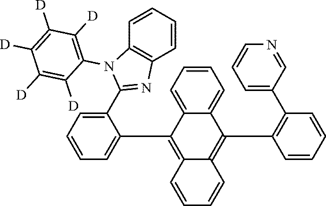 [SMILES: [2H]c1c([2H])c([2H])c(-n2c(-c3ccccc3-c3c4ccccc4c(-c4ccccc4-c4cccnc4)c4ccccc34)nc3ccccc32)c([2H])c1[2H]]

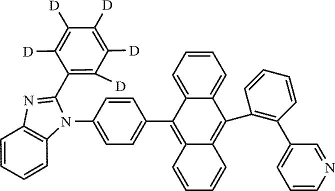 [SMILES: [2H]c1c([2H])c([2H])c(-c2nc3ccccc3n2-c2ccc(-c3c4ccccc4c(-c4ccccc4-c4cccnc4)c4ccccc34)cc2)c([2H])c1[2H]]

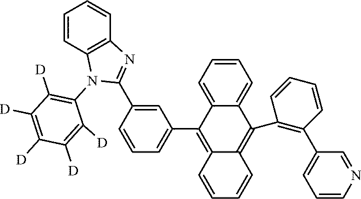 [SMILES: [2H]c1c([2H])c([2H])c(-n2c(-c3cccc(-c4c5ccccc5c(-c5ccccc5-c5cccnc5)c5ccccc45)c3)nc3ccccc32)c([2H])c1[2H]]

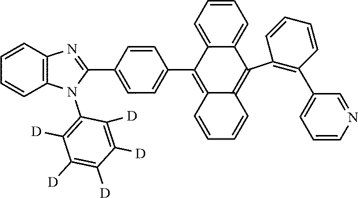 [SMILES: [2H]c1c([2H])c([2H])c(-n2c(-c3ccc(-c4c5ccccc5c(-c5ccccc5-c5cccnc5)c5ccccc45)cc3)nc3ccccc32)c([2H])c1[2H]]

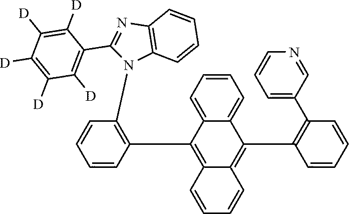 [SMILES: [2H]c1c([2H])c([2H])c(-c2nc3ccccc3n2-c2ccccc2-c2c3ccccc3c(-c3ccccc3-c3cccnc3)c3ccccc23)c([2H])c1[2H]]

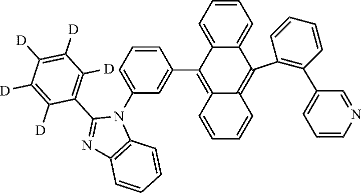 [SMILES: [2H]c1c([2H])c([2H])c(-c2nc3ccccc3n2-c2cccc(-c3c4ccccc4c(-c4ccccc4-c4cccnc4)c4ccccc34)c2)c([2H])c1[2H]]

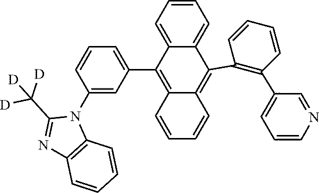 [SMILES: [2H]C([2H])([2H])c1nc2ccccc2n1-c1cccc(-c2c3ccccc3c(-c3ccccc3-c3cccnc3)c3ccccc23)c1]

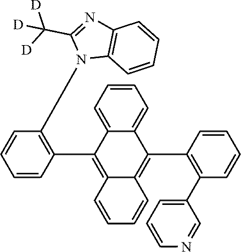 [SMILES: [2H]C([2H])([2H])c1nc2ccccc2n1-c1ccccc1-c1c2ccccc2c(-c2ccccc2-c2cccnc2)c2ccccc12]

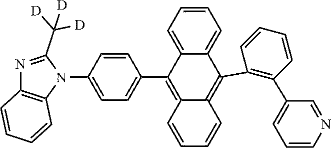 [SMILES: [2H]C([2H])([2H])c1nc2ccccc2n1-c1ccc(-c2c3ccccc3c(-c3ccccc3-c3cccnc3)c3ccccc23)cc1]

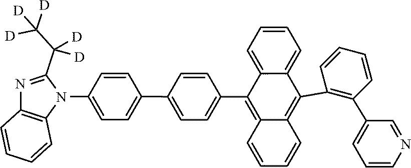 [SMILES: [2H]C([2H])([2H])C([2H])([2H])c1nc2ccccc2n1-c1ccc(-c2ccc(-c3c4ccccc4c(-c4ccccc4-c4cccnc4)c4ccccc34)cc2)cc1]

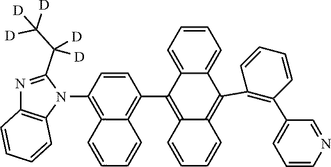 [SMILES: [2H]C([2H])([2H])C([2H])([2H])c1nc2ccccc2n1-c1ccc(-c2c3ccccc3c(-c3ccccc3-c3cccnc3)c3ccccc23)c2ccccc12]

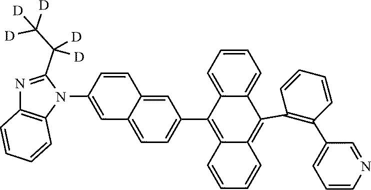 [SMILES: [2H]C([2H])([2H])C([2H])([2H])c1nc2ccccc2n1-c1ccc2cc(-c3c4ccccc4c(-c4ccccc4-c4cccnc4)c4ccccc34)ccc2c1]

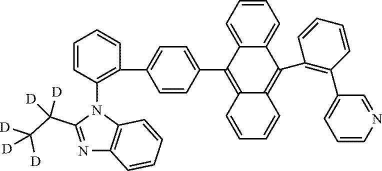 [SMILES: [2H]C([2H])([2H])C([2H])([2H])c1nc2ccccc2n1-c1ccccc1-c1ccc(-c2c3ccccc3c(-c3ccccc3-c3cccnc3)c3ccccc23)cc1]

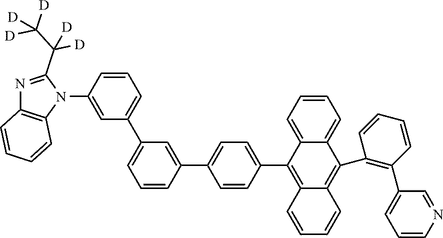 [SMILES: [2H]C([2H])([2H])C([2H])([2H])c1nc2ccccc2n1-c1cccc(-c2cccc(-c3ccc(-c4c5ccccc5c(-c5ccccc5-c5cccnc5)c5ccccc45)cc3)c2)c1]

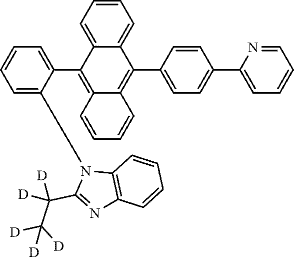 [SMILES: [2H]C([2H])([2H])C([2H])([2H])c1nc2ccccc2n1-c1ccccc1-c1c2ccccc2c(-c2ccc(-c3ccccn3)cc2)c2ccccc12]

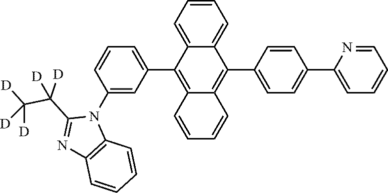 [SMILES: [2H]C([2H])([2H])C([2H])([2H])c1nc2ccccc2n1-c1cccc(-c2c3ccccc3c(-c3ccc(-c4ccccn4)cc3)c3ccccc23)c1]

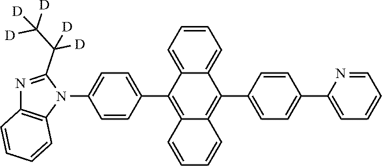 [SMILES: [2H]C([2H])([2H])C([2H])([2H])c1nc2ccccc2n1-c1ccc(-c2c3ccccc3c(-c3ccc(-c4ccccn4)cc3)c3ccccc23)cc1]

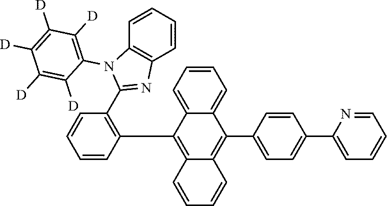 [SMILES: [2H]c1c([2H])c([2H])c(-n2c(-c3ccccc3Cc3c4ccccc4c(-c4ccc(-c5ccccn5)cc4)c4ccccc34)nc3ccccc32)c([2H])c1[2H]]

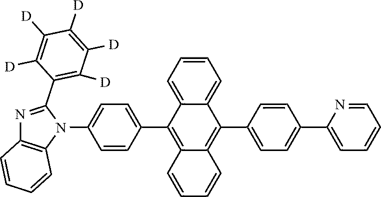 [SMILES: [2H]c1c([2H])c([2H])c(-c2nc3ccccc3n2-c2ccc(-c3c4ccccc4c(-c4ccc(-c5ccccn5)cc4)c4ccccc34)cc2)c([2H])c1[2H]]

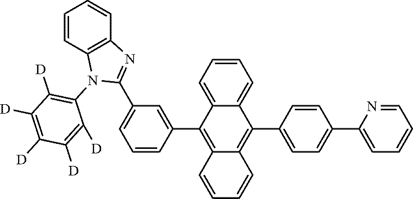 [SMILES: [2H]c1c([2H])c([2H])c(-n2c(-c3cccc(-c4c5ccccc5c(-c5ccc(-c6ccccn6)cc5)c5ccccc45)c3)nc3ccccc32)c([2H])c1[2H]]

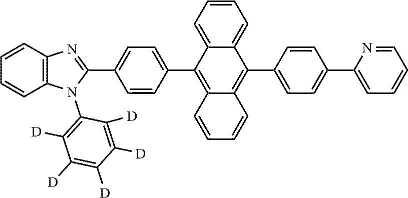 [SMILES: [2H]c1c([2H])c([2H])c(-n2c(-c3ccc(-c4c5ccccc5c(-c5ccc(-c6ccccn6)cc5)c5ccccc45)cc3)nc3ccccc32)c([2H])c1[2H]]

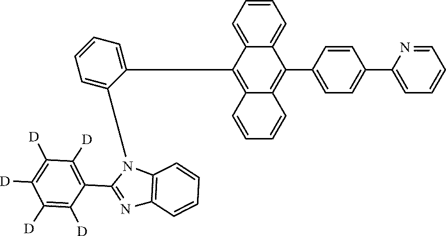 [SMILES: [2H]c1c([2H])c([2H])c(-c2nc3ccccc3n2CCc2ccccc2CCc2c3ccccc3c(-c3ccc(-c4ccccn4)cc3)c3ccccc23)c([2H])c1[2H]]

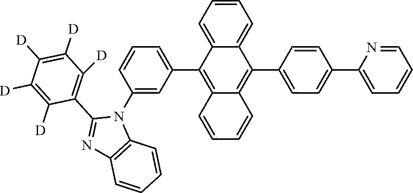 [SMILES: [2H]c1c([2H])c([2H])c(-c2nc3ccccc3n2-c2cccc(-c3c4ccccc4c(-c4ccc(-c5ccccn5)cc4)c4ccccc34)c2)c([2H])c1[2H]]

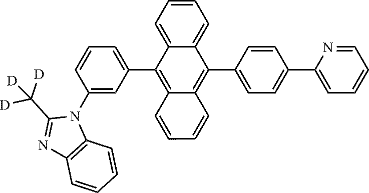 [SMILES: [2H]C([2H])([2H])c1nc2ccccc2n1-c1cccc(-c2c3ccccc3c(-c3ccc(-c4ccccn4)cc3)c3ccccc23)c1]

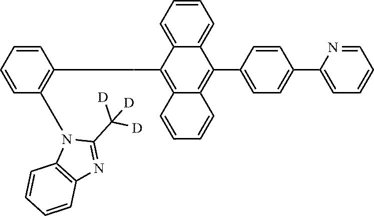 [SMILES: [2H]C([2H])([2H])c1nc2ccccc2n1Cc1ccccc1CCc1c2ccccc2c(-c2ccc(-c3ccccn3)cc2)c2ccccc12]

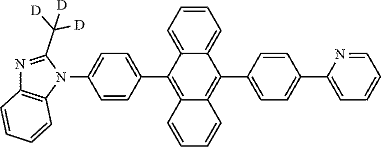 [SMILES: [2H]C([2H])([2H])c1nc2ccccc2n1-c1ccc(-c2c3ccccc3c(-c3ccc(-c4ccccn4)cc3)c3ccccc23)cc1]

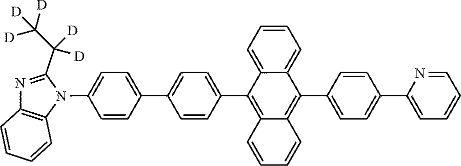 [SMILES: [2H]C([2H])([2H])C([2H])([2H])c1nc2ccccc2n1-c1ccc(-c2ccc(-c3c4ccccc4c(-c4ccc(-c5ccccn5)cc4)c4ccccc34)cc2)cc1]

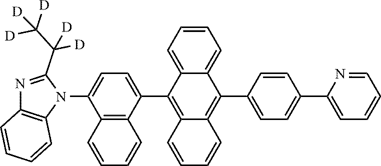 [SMILES: [2H]C([2H])([2H])C([2H])([2H])c1nc2ccccc2n1-c1ccc(-c2c3ccccc3c(-c3ccc(-c4ccccn4)cc3)c3ccccc23)c2ccccc12]

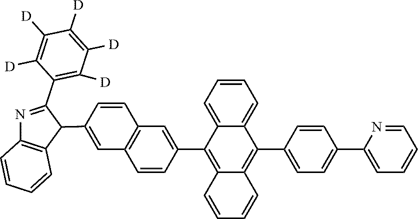 [SMILES: [2H]c1c([2H])c([2H])c(C2=Nc3ccccc3C2c2ccc3cc(-c4c5ccccc5c(-c5ccc(-c6ccccn6)cc5)c5ccccc45)ccc3c2)c([2H])c1[2H]]

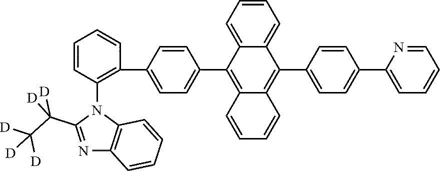 [SMILES: [2H]C([2H])([2H])C([2H])([2H])c1nc2ccccc2n1-c1ccccc1-c1ccc(-c2c3ccccc3c(-c3ccc(-c4ccccn4)cc3)c3ccccc23)cc1]

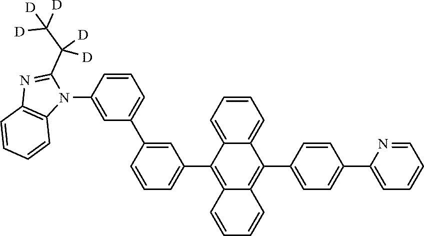 [SMILES: [2H]C([2H])([2H])C([2H])([2H])c1nc2ccccc2n1-c1cccc(-c2cccc(-c3c4ccccc4c(-c4ccc(-c5ccccn5)cc4)c4ccccc34)c2)c1]

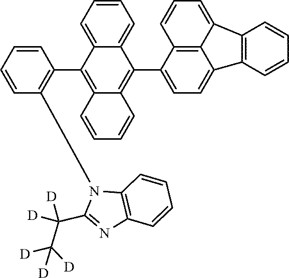 [SMILES: [2H]C([2H])([2H])C([2H])([2H])c1nc2ccccc2n1-c1ccccc1-c1c2ccccc2c(-c2ccc3c4c(cccc24)-c2ccccc2-3)c2ccccc12]

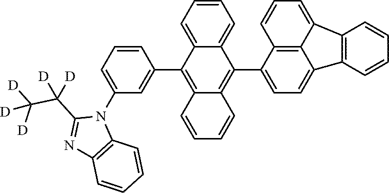 [SMILES: [2H]C([2H])([2H])C([2H])([2H])c1nc2ccccc2n1-c1cccc(-c2c3ccccc3c(-c3ccc4c5c(cccc35)-c3ccccc3-4)c3ccccc23)c1]

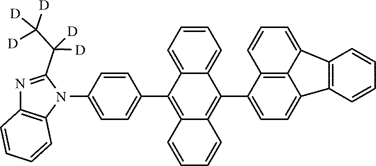 [SMILES: [2H]C([2H])([2H])C([2H])([2H])c1nc2ccccc2n1-c1ccc(-c2c3ccccc3c(-c3ccc4c5c(cccc35)-c3ccccc3-4)c3ccccc23)cc1]

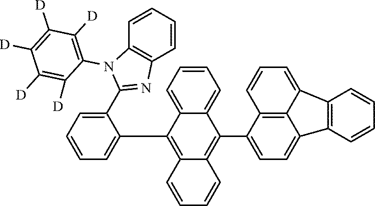 [SMILES: [2H]c1c([2H])c([2H])c(-n2c(-c3ccccc3-c3c4ccccc4c(-c4ccc5c6c(cccc46)-c4ccccc4-5)c4ccccc34)nc3ccccc32)c([2H])c1[2H]]

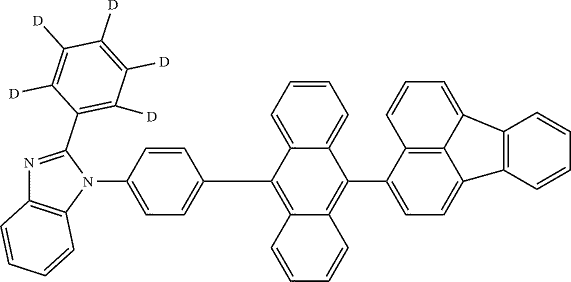 [SMILES: [2H]c1c([2H])c([2H])c(-c2nc3ccccc3n2-c2ccc(-c3c4ccccc4c(-c4ccc5c6c(cccc46)-c4ccccc4-5)c4ccccc34)cc2)c([2H])c1[2H]]

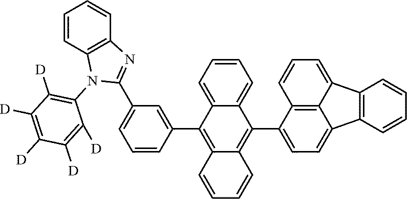 [SMILES: [2H]c1c([2H])c([2H])c(-n2c(-c3cccc(-c4c5ccccc5c(-c5ccc6c7c(cccc57)-c5ccccc5-6)c5ccccc45)c3)nc3ccccc32)c([2H])c1[2H]]

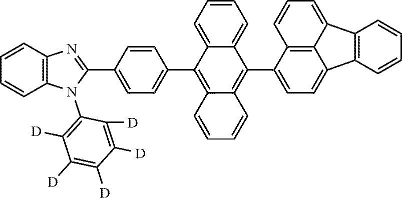 [SMILES: [2H]c1c([2H])c([2H])c(-n2c(-c3ccc(-c4c5ccccc5c(-c5ccc6c7c(cccc57)-c5ccccc5-6)c5ccccc45)cc3)nc3ccccc32)c([2H])c1[2H]]

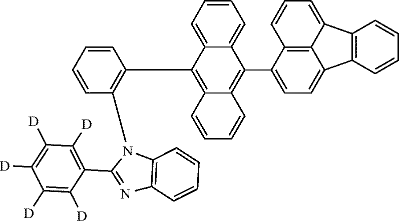 [SMILES: [2H]c1c([2H])c([2H])c(-c2nc3ccccc3n2-c2ccccc2-c2c3ccccc3c(-c3ccc4c5c(cccc35)-c3ccccc3-4)c3ccccc23)c([2H])c1[2H]]

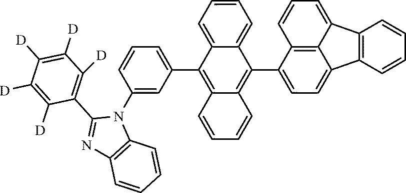 [SMILES: [2H]c1c([2H])c([2H])c(-c2nc3ccccc3n2-c2cccc(-c3c4ccccc4c(-c4ccc5c6c(cccc46)-c4ccccc4-5)c4ccccc34)c2)c([2H])c1[2H]]

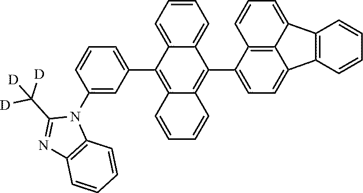 [SMILES: [2H]C([2H])([2H])c1nc2ccccc2n1-c1cccc(-c2c3ccccc3c(-c3ccc4c5c(cccc35)-c3ccccc3-4)c3ccccc23)c1]

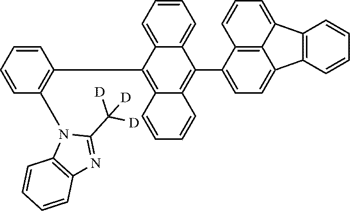 [SMILES: [2H]C([2H])([2H])c1nc2ccccc2n1-c1ccccc1-c1c2ccccc2c(-c2ccc3c4c(cccc24)-c2ccccc2-3)c2ccccc12]

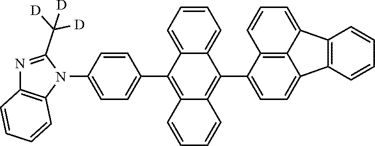 [SMILES: [2H]C([2H])([2H])c1nc2ccccc2n1-c1ccc(-c2c3ccccc3c(-c3ccc4c5c(cccc35)-c3ccccc3-4)c3ccccc23)cc1]

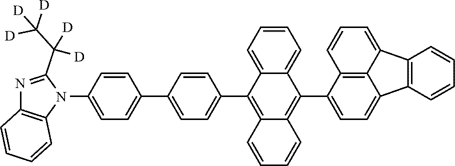 [SMILES: [2H]C([2H])([2H])C([2H])([2H])c1nc2ccccc2n1-c1ccc(-c2ccc(-c3c4ccccc4c(-c4ccc5c6c(cccc46)-c4ccccc4-5)c4ccccc34)cc2)cc1]

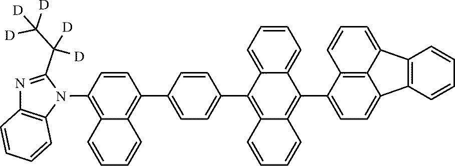 [SMILES: [2H]C([2H])([2H])C([2H])([2H])c1nc2ccccc2n1-c1ccc(-c2ccc(-c3c4ccccc4c(-c4ccc5c6c(cccc46)-c4ccccc4-5)c4ccccc34)cc2)c2ccccc12]

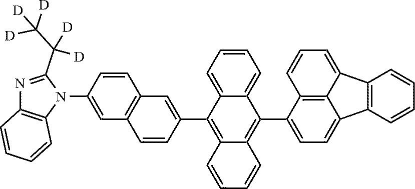 [SMILES: [2H]C([2H])([2H])C([2H])([2H])c1nc2ccccc2n1-c1ccc2cc(-c3c4ccccc4c(-c4ccc5c6c(cccc46)-c4ccccc4-5)c4ccccc34)ccc2c1]

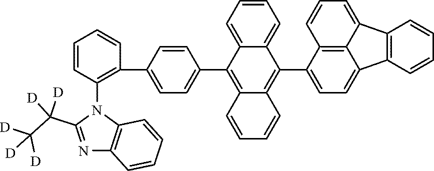 [SMILES: [2H]C([2H])([2H])C([2H])([2H])c1nc2ccccc2n1-c1ccccc1-c1ccc(-c2c3ccccc3c(-c3ccc4c5c(cccc35)-c3ccccc3-4)c3ccccc23)cc1]

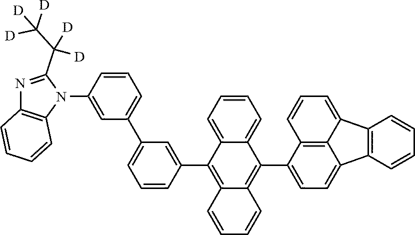 [SMILES: [2H]C([2H])([2H])C([2H])([2H])c1nc2ccccc2n1-c1cccc(-c2cccc(-c3c4ccccc4c(-c4ccc5c6c(cccc46)-c4ccccc4-5)c4ccccc34)c2)c1]

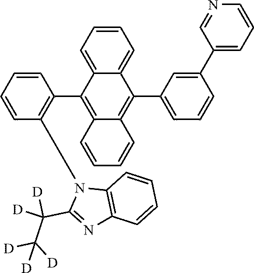 [SMILES: [2H]C([2H])([2H])C([2H])([2H])c1nc2ccccc2n1-c1ccccc1-c1c2ccccc2c(-c2cccc(-c3cccnc3)c2)c2ccccc12]

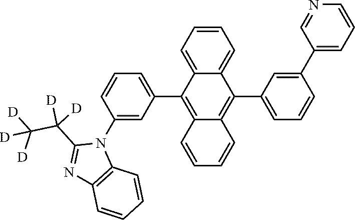 [SMILES: [2H]C([2H])([2H])C([2H])([2H])c1nc2ccccc2n1-c1cccc(-c2c3ccccc3c(-c3cccc(-c4cccnc4)c3)c3ccccc23)c1]

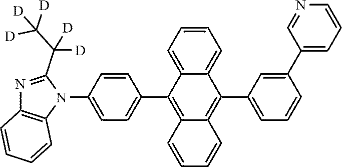 [SMILES: [2H]C([2H])([2H])C([2H])([2H])c1nc2ccccc2n1-c1ccc(-c2c3ccccc3c(-c3cccc(-c4cccnc4)c3)c3ccccc23)cc1]

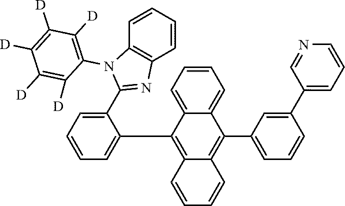 [SMILES: [2H]c1c([2H])c([2H])c(-n2c(-c3ccccc3-c3c4ccccc4c(-c4cccc(-c5cccnc5)c4)c4ccccc34)nc3ccccc32)c([2H])c1[2H]]

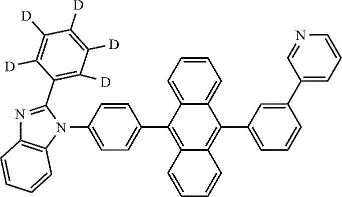 [SMILES: [2H]c1c([2H])c([2H])c(-c2nc3ccccc3n2-c2ccc(-c3c4ccccc4c(-c4cccc(-c5cccnc5)c4)c4ccccc34)cc2)c([2H])c1[2H]]

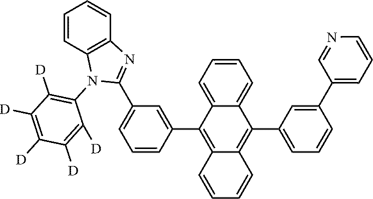 [SMILES: [2H]c1c([2H])c([2H])c(-n2c(-c3cccc(-c4c5ccccc5c(-c5cccc(-c6cccnc6)c5)c5ccccc45)c3)nc3ccccc32)c([2H])c1[2H]]

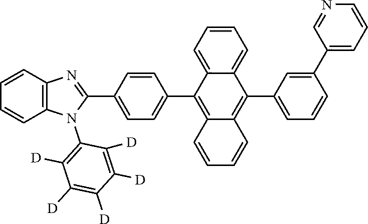 [SMILES: [2H]c1c([2H])c([2H])c(-n2c(-c3ccc(-c4c5ccccc5c(-c5cccc(-c6cccnc6)c5)c5ccccc45)cc3)nc3ccccc32)c([2H])c1[2H]]

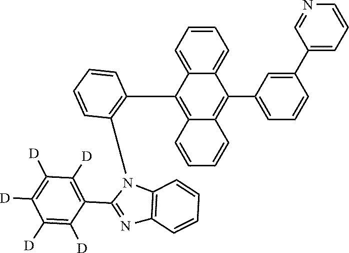 [SMILES: [2H]c1c([2H])c([2H])c(-c2nc3ccccc3n2-c2ccccc2-c2c3ccccc3c(-c3cccc(-c4cccnc4)c3)c3ccccc23)c([2H])c1[2H]]

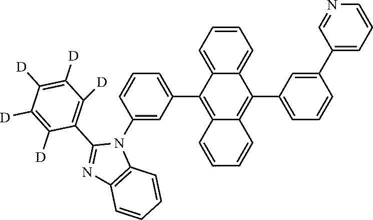 [SMILES: [2H]c1c([2H])c([2H])c(-c2nc3ccccc3n2-c2cccc(-c3c4ccccc4c(-c4cccc(-c5cccnc5)c4)c4ccccc34)c2)c([2H])c1[2H]]

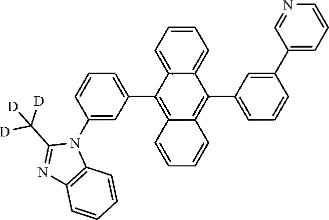 [SMILES: [2H]C([2H])([2H])c1nc2ccccc2n1-c1cccc(-c2c3ccccc3c(-c3cccc(-c4cccnc4)c3)c3ccccc23)c1]

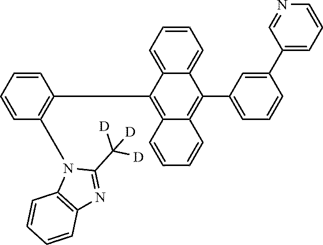 [SMILES: [2H]C([2H])([2H])c1nc2ccccc2n1-c1ccccc1-c1c2ccccc2c(-c2cccc(-c3cccnc3)c2)c2ccccc12]

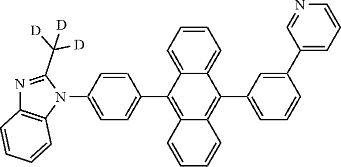 [SMILES: [2H]C([2H])([2H])c1nc2ccccc2n1-c1ccc(-c2c3ccccc3c(-c3cccc(-c4cccnc4)c3)c3ccccc23)cc1]

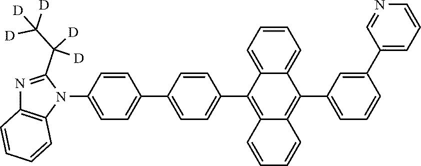 [SMILES: [2H]C([2H])([2H])C([2H])([2H])c1nc2ccccc2n1-c1ccc(-c2ccc(-c3c4ccccc4c(-c4cccc(-c5cccnc5)c4)c4ccccc34)cc2)cc1]

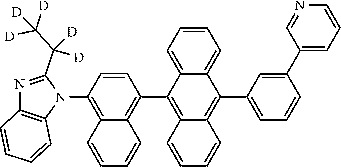 [SMILES: [2H]C([2H])([2H])C([2H])([2H])c1nc2ccccc2n1-c1ccc(-c2c3ccccc3c(-c3cccc(-c4cccnc4)c3)c3ccccc23)c2ccccc12]

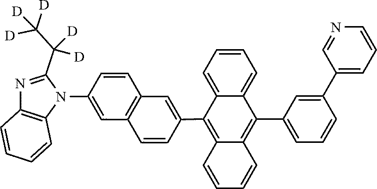 [SMILES: [2H]C([2H])([2H])C([2H])([2H])c1nc2ccccc2n1-c1ccc2cc(-c3c4ccccc4c(-c4cccc(-c5cccnc5)c4)c4ccccc34)ccc2c1]

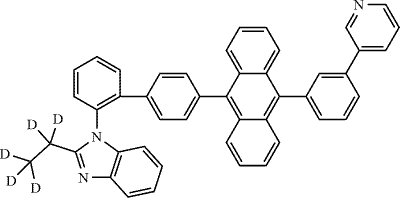 [SMILES: [2H]C([2H])([2H])C([2H])([2H])c1nc2ccccc2n1-c1ccccc1-c1ccc(-c2c3ccccc3c(-c3cccc(-c4cccnc4)c3)c3ccccc23)cc1]

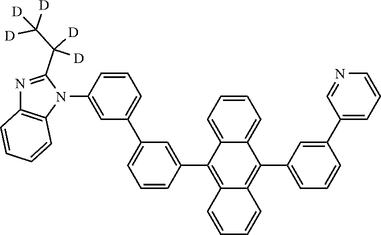 [SMILES: [2H]C([2H])([2H])C([2H])([2H])c1nc2ccccc2n1-c1cccc(-c2cccc(-c3c4ccccc4c(-c4cccc(-c5cccnc5)c4)c4ccccc34)c2)c1]

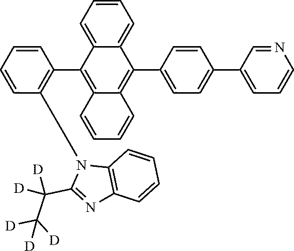 [SMILES: [2H]C([2H])([2H])C([2H])([2H])c1nc2ccccc2n1-c1ccccc1-c1c2ccccc2c(-c2ccc(-c3cccnc3)cc2)c2ccccc12]

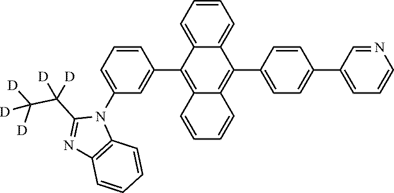 [SMILES: [2H]C([2H])([2H])C([2H])([2H])c1nc2ccccc2n1-c1cccc(-c2c3ccccc3c(-c3ccc(-c4cccnc4)cc3)c3ccccc23)c1]

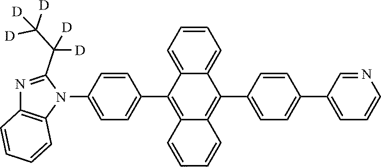 [SMILES: [2H]C([2H])([2H])C([2H])([2H])c1nc2ccccc2n1-c1ccc(-c2c3ccccc3c(-c3ccc(-c4cccnc4)cc3)c3ccccc23)cc1]

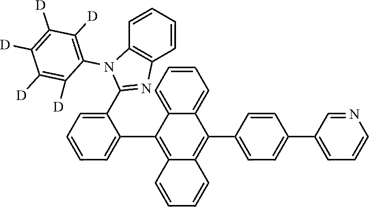 [SMILES: [2H]c1c([2H])c([2H])c(-n2c(-c3ccccc3-c3c4ccccc4c(-c4ccc(-c5cccnc5)cc4)c4ccccc34)nc3ccccc32)c([2H])c1[2H]]

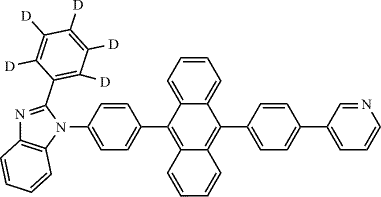 [SMILES: [2H]c1c([2H])c([2H])c(-c2nc3ccccc3n2-c2ccc(-c3c4ccccc4c(-c4ccc(-c5cccnc5)cc4)c4ccccc34)cc2)c([2H])c1[2H]]

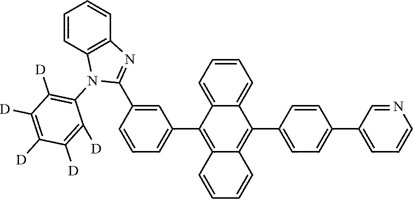 [SMILES: [2H]c1c([2H])c([2H])c(-n2c(-c3cccc(-c4c5ccccc5c(-c5ccc(-c6cccnc6)cc5)c5ccccc45)c3)nc3ccccc32)c([2H])c1[2H]]

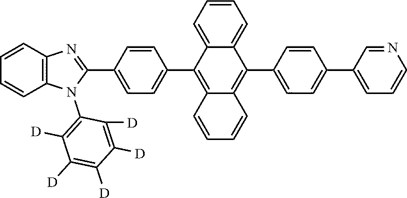 [SMILES: [2H]c1c([2H])c([2H])c(-n2c(-c3ccc(-c4c5ccccc5c(-c5ccc(-c6cccnc6)cc5)c5ccccc45)cc3)nc3ccccc32)c([2H])c1[2H]]

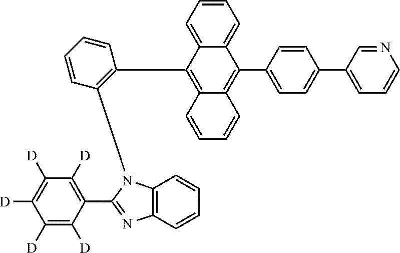 [SMILES: [2H]c1c([2H])c([2H])c(-c2nc3ccccc3n2-c2ccccc2-c2c3ccccc3c(-c3ccc(-c4cccnc4)cc3)c3ccccc23)c([2H])c1[2H]]

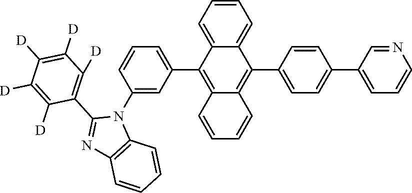 [SMILES: [2H]c1c([2H])c([2H])c(-c2nc3ccccc3n2-c2cccc(-c3c4ccccc4c(-c4ccc(-c5cccnc5)cc4)c4ccccc34)c2)c([2H])c1[2H]]

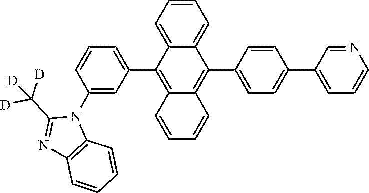 [SMILES: [2H]C([2H])([2H])c1nc2ccccc2n1-c1cccc(-c2c3ccccc3c(-c3ccc(-c4cccnc4)cc3)c3ccccc23)c1]

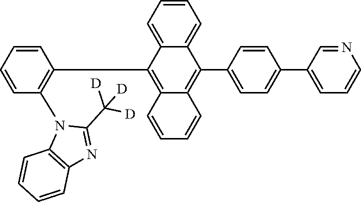 [SMILES: [2H]C([2H])([2H])c1nc2ccccc2n1-c1ccccc1-c1c2ccccc2c(-c2ccc(-c3cccnc3)cc2)c2ccccc12]

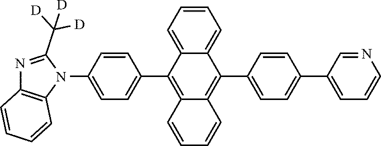 [SMILES: [2H]C([2H])([2H])c1nc2ccccc2n1-c1ccc(-c2c3ccccc3c(-c3ccc(-c4cccnc4)cc3)c3ccccc23)cc1]

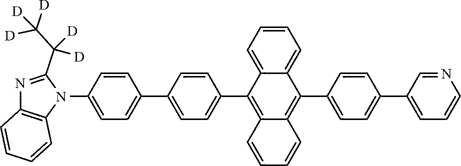 [SMILES: [2H]C([2H])([2H])C([2H])([2H])c1nc2ccccc2n1-c1ccc(-c2ccc(-c3c4ccccc4c(-c4ccc(-c5cccnc5)cc4)c4ccccc34)cc2)cc1]

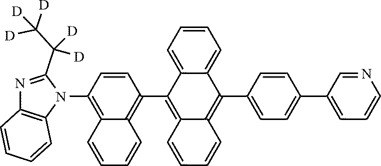 [SMILES: [2H]C([2H])([2H])C([2H])([2H])c1nc2ccccc2n1-c1ccc(-c2c3ccccc3c(-c3ccc(-c4cccnc4)cc3)c3ccccc23)c2ccccc12]

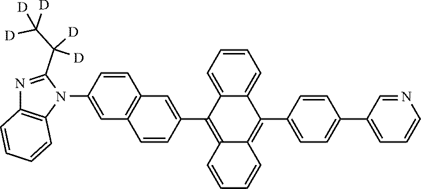 [SMILES: [2H]C([2H])([2H])C([2H])([2H])c1nc2ccccc2n1-c1ccc2cc(-c3c4ccccc4c(-c4ccc(-c5cccnc5)cc4)c4ccccc34)ccc2c1]

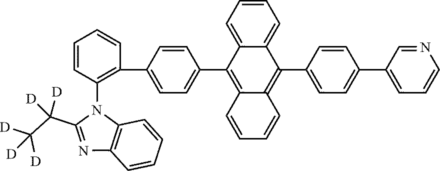 [SMILES: [2H]C([2H])([2H])C([2H])([2H])c1nc2ccccc2n1-c1ccccc1-c1ccc(-c2c3ccccc3c(-c3ccc(-c4cccnc4)cc3)c3ccccc23)cc1]

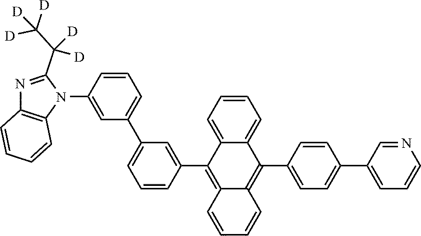 [SMILES: [2H]C([2H])([2H])C([2H])([2H])c1nc2ccccc2n1-c1cccc(-c2cccc(-c3c4ccccc4c(-c4ccc(-c5cccnc5)cc4)c4ccccc34)c2)c1]

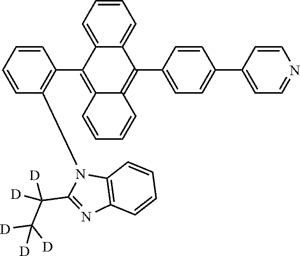 [SMILES: [2H]C([2H])([2H])C([2H])([2H])c1nc2ccccc2n1-c1ccccc1-c1c2ccccc2c(-c2ccc(-c3ccncc3)cc2)c2ccccc12]

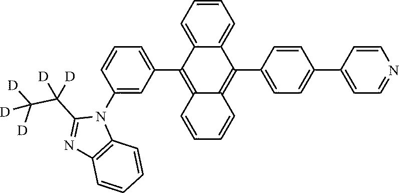 [SMILES: [2H]C([2H])([2H])C([2H])([2H])c1nc2ccccc2n1-c1cccc(-c2c3ccccc3c(-c3ccc(-c4ccncc4)cc3)c3ccccc23)c1]

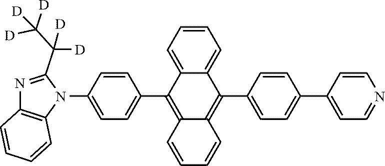 [SMILES: [2H]C([2H])([2H])C([2H])([2H])c1nc2ccccc2n1-c1ccc(-c2c3ccccc3c(-c3ccc(-c4ccncc4)cc3)c3ccccc23)cc1]

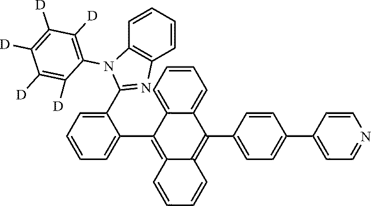 [SMILES: [2H]c1c([2H])c([2H])c(-n2c(-c3ccccc3-c3c4ccccc4c(-c4ccc(-c5ccncc5)cc4)c4ccccc34)nc3ccccc32)c([2H])c1[2H]]

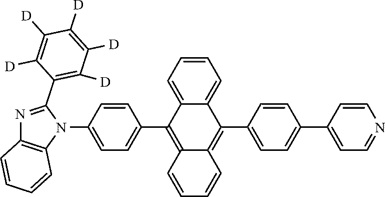 [SMILES: [2H]c1c([2H])c([2H])c(-c2nc3ccccc3n2-c2ccc(-c3c4ccccc4c(-c4ccc(-c5ccncc5)cc4)c4ccccc34)cc2)c([2H])c1[2H]]

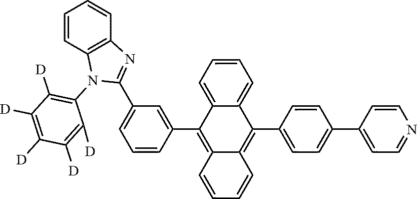 [SMILES: [2H]c1c([2H])c([2H])c(-n2c(-c3cccc(-c4c5ccccc5c(-c5ccc(-c6ccncc6)cc5)c5ccccc45)c3)nc3ccccc32)c([2H])c1[2H]]

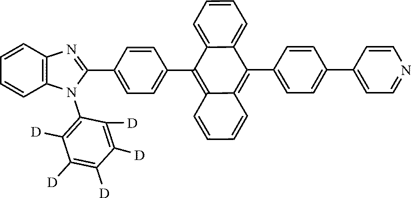 [SMILES: [2H]c1c([2H])c([2H])c(-n2c(-c3ccc(-c4c5ccccc5c(-c5ccc(-c6ccncc6)cc5)c5ccccc45)cc3)nc3ccccc32)c([2H])c1[2H]]

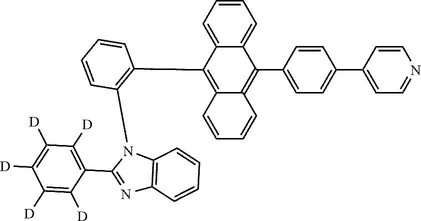 [SMILES: [2H]c1c([2H])c([2H])c(-c2nc3ccccc3n2-c2ccccc2-c2c3ccccc3c(-c3ccc(-c4ccncc4)cc3)c3ccccc23)c([2H])c1[2H]]

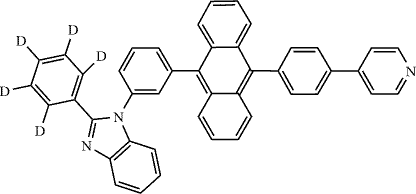 [SMILES: [2H]c1c([2H])c([2H])c(-c2nc3ccccc3n2-c2cccc(-c3c4ccccc4c(-c4ccc(-c5ccncc5)cc4)c4ccccc34)c2)c([2H])c1[2H]]

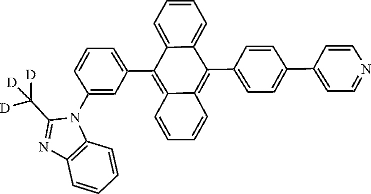 [SMILES: [2H]C([2H])([2H])c1nc2ccccc2n1-c1cccc(-c2c3ccccc3c(-c3ccc(-c4ccncc4)cc3)c3ccccc23)c1]

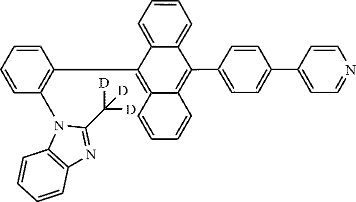 [SMILES: [2H]C([2H])([2H])c1nc2ccccc2n1-c1ccccc1-c1c2ccccc2c(-c2ccc(-c3ccncc3)cc2)c2ccccc12]

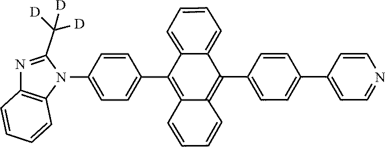 [SMILES: [2H]C([2H])([2H])c1nc2ccccc2n1-c1ccc(-c2c3ccccc3c(-c3ccc(-c4ccncc4)cc3)c3ccccc23)cc1]

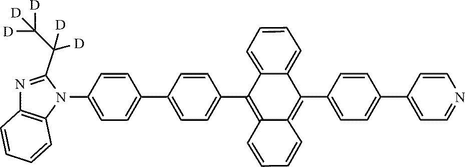 [SMILES: [2H]C([2H])([2H])C([2H])([2H])c1nc2ccccc2n1-c1ccc(-c2ccc(-c3c4ccccc4c(-c4ccc(-c5ccncc5)cc4)c4ccccc34)cc2)cc1]

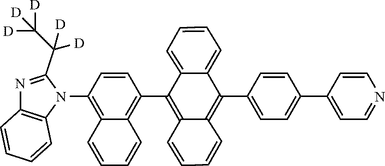 [SMILES: [2H]C([2H])([2H])C([2H])([2H])c1nc2ccccc2n1-c1ccc(-c2c3ccccc3c(-c3ccc(-c4ccncc4)cc3)c3ccccc23)c2ccccc12]

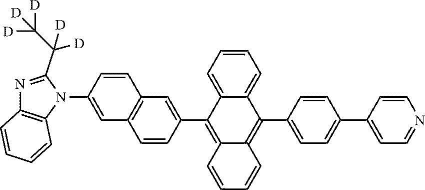 [SMILES: [2H]C([2H])([2H])C([2H])([2H])c1nc2ccccc2n1-c1ccc2cc(-c3c4ccccc4c(-c4ccc(-c5ccncc5)cc4)c4ccccc34)ccc2c1]

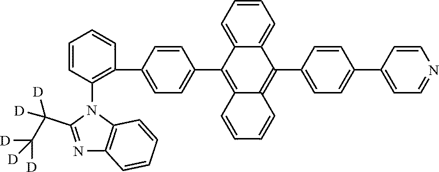 [SMILES: [2H]C([2H])([2H])C([2H])([2H])c1nc2ccccc2n1-c1ccccc1-c1ccc(-c2c3ccccc3c(-c3ccc(-c4ccncc4)cc3)c3ccccc23)cc1]

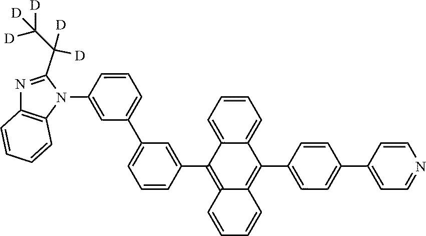 [SMILES: [2H]C([2H])([2H])C([2H])([2H])c1nc2ccccc2n1-c1cccc(-c2cccc(-c3c4ccccc4c(-c4ccc(-c5ccncc5)cc4)c4ccccc34)c2)c1]

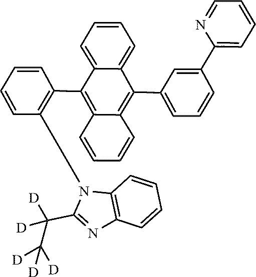 [SMILES: [2H]C([2H])([2H])C([2H])([2H])c1nc2ccccc2n1-c1ccccc1-c1c2ccccc2c(-c2cccc(-c3ccccn3)c2)c2ccccc12]

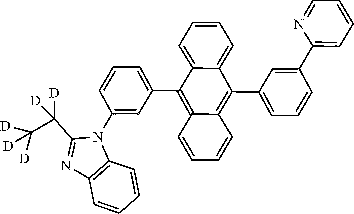 [SMILES: [2H]C([2H])([2H])C([2H])([2H])c1nc2ccccc2n1-c1cccc(-c2c3ccccc3c(-c3cccc(-c4ccccn4)c3)c3ccccc23)c1]

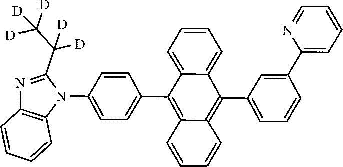 [SMILES: [2H]C([2H])([2H])C([2H])([2H])c1nc2ccccc2n1-c1ccc(-c2c3ccccc3c(-c3cccc(-c4ccccn4)c3)c3ccccc23)cc1]

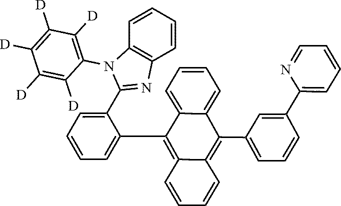 [SMILES: [2H]c1c([2H])c([2H])c(-n2c(-c3ccccc3-c3c4ccccc4c(-c4cccc(-c5ccccn5)c4)c4ccccc34)nc3ccccc32)c([2H])c1[2H]]

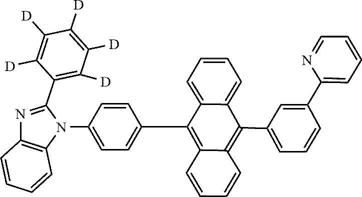 [SMILES: [2H]c1c([2H])c([2H])c(-c2nc3ccccc3n2-c2ccc(-c3c4ccccc4c(-c4cccc(-c5ccccn5)c4)c4ccccc34)cc2)c([2H])c1[2H]]

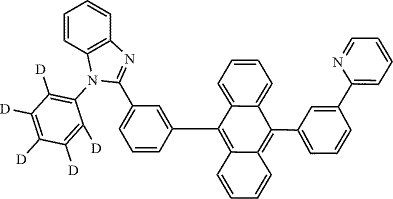 [SMILES: [2H]c1c([2H])c([2H])c(-n2c(-c3cccc(-c4c5ccccc5c(-c5cccc(-c6ccccn6)c5)c5ccccc45)c3)nc3ccccc32)c([2H])c1[2H]]

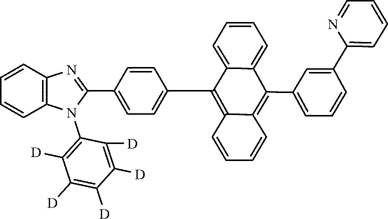 [SMILES: [2H]c1c([2H])c([2H])c(-n2c(-c3ccc(-c4c5ccccc5c(-c5cccc(-c6ccccn6)c5)c5ccccc45)cc3)nc3ccccc32)c([2H])c1[2H]]

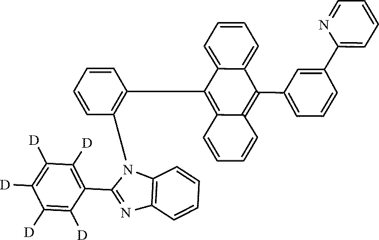 [SMILES: [2H]c1c([2H])c([2H])c(-c2nc3ccccc3n2-c2ccccc2-c2c3ccccc3c(-c3cccc(-c4ccccn4)c3)c3ccccc23)c([2H])c1[2H]]

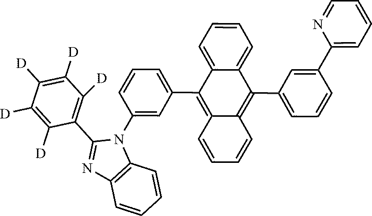 [SMILES: [2H]c1c([2H])c([2H])c(-c2nc3ccccc3n2-c2cccc(-c3c4ccccc4c(-c4cccc(-c5ccccn5)c4)c4ccccc34)c2)c([2H])c1[2H]]

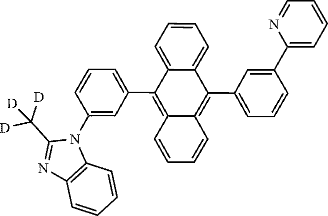 [SMILES: [2H]C([2H])([2H])c1nc2ccccc2n1-c1cccc(-c2c3ccccc3c(-c3cccc(-c4ccccn4)c3)c3ccccc23)c1]

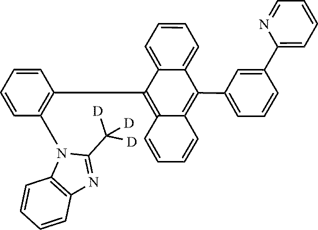 [SMILES: [2H]C([2H])([2H])c1nc2ccccc2n1-c1ccccc1-c1c2ccccc2c(-c2cccc(-c3ccccn3)c2)c2ccccc12]

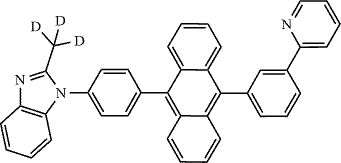 [SMILES: [2H]C([2H])([2H])c1nc2ccccc2n1-c1ccc(-c2c3ccccc3c(-c3cccc(-c4ccccn4)c3)c3ccccc23)cc1]

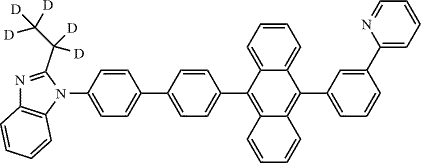 [SMILES: [2H]C([2H])([2H])C([2H])([2H])c1nc2ccccc2n1-c1ccc(-c2ccc(-c3c4ccccc4c(-c4cccc(-c5ccccn5)c4)c4ccccc34)cc2)cc1]

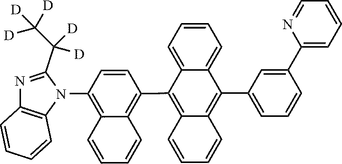 [SMILES: [2H]C([2H])([2H])C([2H])([2H])c1nc2ccccc2n1-c1ccc(-c2c3ccccc3c(-c3cccc(-c4ccccn4)c3)c3ccccc23)c2ccccc12]

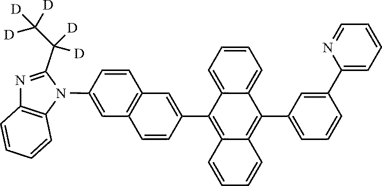 [SMILES: [2H]C([2H])([2H])C([2H])([2H])c1nc2ccccc2n1-c1ccc2cc(-c3c4ccccc4c(-c4cccc(-c5ccccn5)c4)c4ccccc34)ccc2c1]

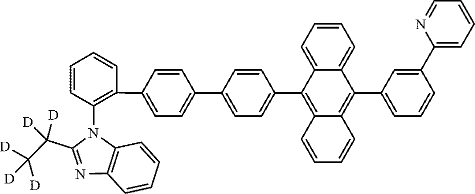 [SMILES: [2H]C([2H])([2H])C([2H])([2H])c1nc2ccccc2n1-c1ccccc1-c1ccc(-c2ccc(-c3c4ccccc4c(-c4cccc(-c5ccccn5)c4)c4ccccc34)cc2)cc1]

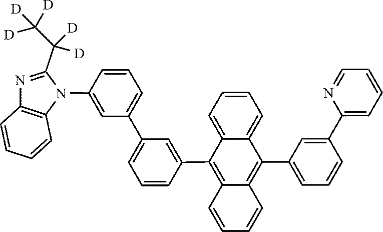 [SMILES: [2H]C([2H])([2H])C([2H])([2H])c1nc2ccccc2n1-c1cccc(-c2cccc(-c3c4ccccc4c(-c4cccc(-c5ccccn5)c4)c4ccccc34)c2)c1]

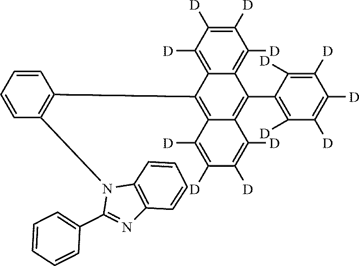 [SMILES: [2H]c1c([2H])c([2H])c(-c2c3c([2H])c([2H])c([2H])c([2H])c3c(-c3ccccc3-n3c(-c4ccccc4)nc4ccccc43)c3c([2H])c([2H])c([2H])c([2H])c23)c([2H])c1[2H]]

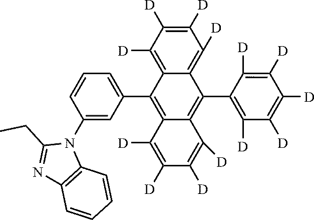 [SMILES: [2H]c1c([2H])c([2H])c(-c2c3c([2H])c([2H])c([2H])c([2H])c3c(-c3cccc(-n4c(CC)nc5ccccc54)c3)c3c([2H])c([2H])c([2H])c([2H])c23)c([2H])c1[2H]]

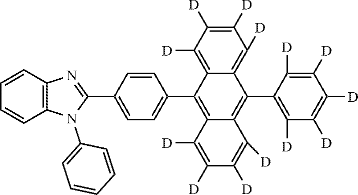 [SMILES: [2H]c1c([2H])c([2H])c(-c2c3c([2H])c([2H])c([2H])c([2H])c3c(-c3ccc(-c4nc5ccccc5n4-c4ccccc4)cc3)c3c([2H])c([2H])c([2H])c([2H])c23)c([2H])c1[2H]]

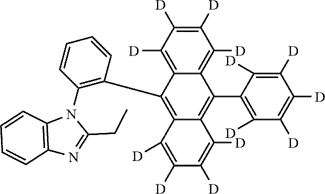 [SMILES: [2H]c1c([2H])c([2H])c(-c2c3c([2H])c([2H])c([2H])c([2H])c3c(-c3ccccc3-n3c(CC)nc4ccccc43)c3c([2H])c([2H])c([2H])c([2H])c23)c([2H])c1[2H]]

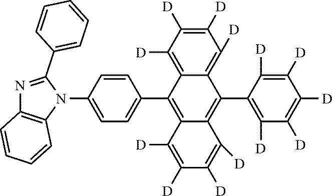 [SMILES: [2H]c1c([2H])c([2H])c(-c2c3c([2H])c([2H])c([2H])c([2H])c3c(-c3ccc(-n4c(-c5ccccc5)nc5ccccc54)cc3)c3c([2H])c([2H])c([2H])c([2H])c23)c([2H])c1[2H]]

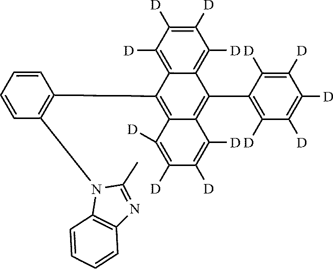 [SMILES: [2H]c1c([2H])c([2H])c(-c2c3c([2H])c([2H])c([2H])c([2H])c3c(-c3ccccc3-n3c(C)nc4ccccc43)c3c([2H])c([2H])c([2H])c([2H])c23)c([2H])c1[2H]]

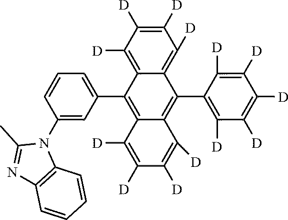 [SMILES: [2H]c1c([2H])c([2H])c(-c2c3c([2H])c([2H])c([2H])c([2H])c3c(-c3cccc(-n4c(C)nc5ccccc54)c3)c3c([2H])c([2H])c([2H])c([2H])c23)c([2H])c1[2H]]

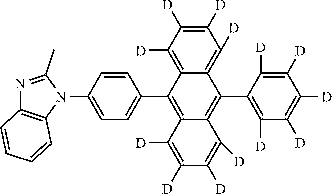 [SMILES: [2H]c1c([2H])c([2H])c(-c2c3c([2H])c([2H])c([2H])c([2H])c3c(-c3ccc(-n4c(C)nc5ccccc54)cc3)c3c([2H])c([2H])c([2H])c([2H])c23)c([2H])c1[2H]]

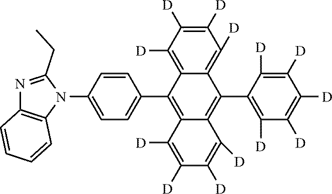 [SMILES: [2H]c1c([2H])c([2H])c(-c2c3c([2H])c([2H])c([2H])c([2H])c3c(-c3ccc(-n4c(CC)nc5ccccc54)cc3)c3c([2H])c([2H])c([2H])c([2H])c23)c([2H])c1[2H]]

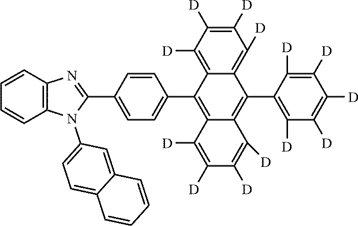 [SMILES: [2H]c1c([2H])c([2H])c(-c2c3c([2H])c([2H])c([2H])c([2H])c3c(-c3ccc(-c4nc5ccccc5n4-c4ccc5ccccc5c4)cc3)c3c([2H])c([2H])c([2H])c([2H])c23)c([2H])c1[2H]]

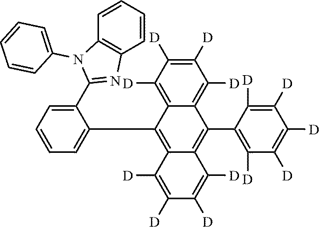 [SMILES: [2H]c1c([2H])c([2H])c(-c2c3c([2H])c([2H])c([2H])c([2H])c3c(-c3ccccc3-c3nc4ccccc4n3-c3ccccc3)c3c([2H])c([2H])c([2H])c([2H])c23)c([2H])c1[2H]]

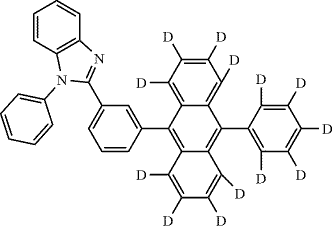 [SMILES: [2H]c1c([2H])c([2H])c(-c2c3c([2H])c([2H])c([2H])c([2H])c3c(-c3cccc(-c4nc5ccccc5n4-c4ccccc4)c3)c3c([2H])c([2H])c([2H])c([2H])c23)c([2H])c1[2H]]

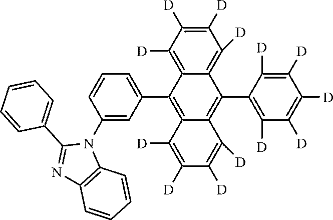 [SMILES: [2H]c1c([2H])c([2H])c(-c2c3c([2H])c([2H])c([2H])c([2H])c3c(-c3cccc(-n4c(-c5ccccc5)nc5ccccc54)c3)c3c([2H])c([2H])c([2H])c([2H])c23)c([2H])c1[2H]]

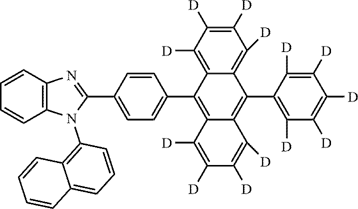 [SMILES: [2H]c1c([2H])c([2H])c(-c2c3c([2H])c([2H])c([2H])c([2H])c3c(-c3ccc(-c4nc5ccccc5n4-c4cccc5ccccc45)cc3)c3c([2H])c([2H])c([2H])c([2H])c23)c([2H])c1[2H]]

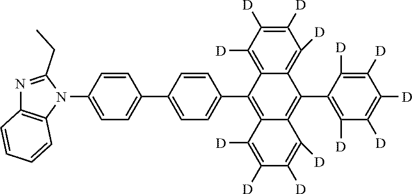 [SMILES: [2H]c1c([2H])c([2H])c(-c2c3c([2H])c([2H])c([2H])c([2H])c3c(-c3ccc(-c4ccc(-n5c(CC)nc6ccccc65)cc4)cc3)c3c([2H])c([2H])c([2H])c([2H])c23)c([2H])c1[2H]]

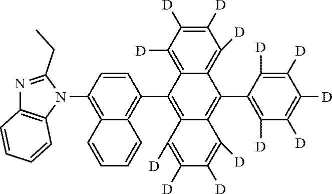 [SMILES: [2H]c1c([2H])c([2H])c(-c2c3c([2H])c([2H])c([2H])c([2H])c3c(-c3ccc(-n4c(CC)nc5ccccc54)c4ccccc34)c3c([2H])c([2H])c([2H])c([2H])c23)c([2H])c1[2H]]

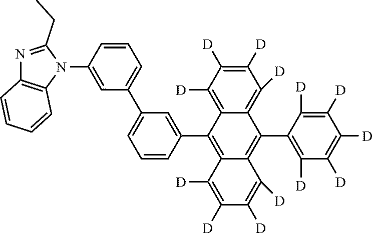 [SMILES: [2H]c1c([2H])c([2H])c(-c2c3c([2H])c([2H])c([2H])c([2H])c3c(-c3cccc(-c4cccc(-n5c(CC)nc6ccccc65)c4)c3)c3c([2H])c([2H])c([2H])c([2H])c23)c([2H])c1[2H]]

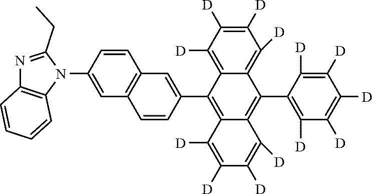 [SMILES: [2H]c1c([2H])c([2H])c(-c2c3c([2H])c([2H])c([2H])c([2H])c3c(-c3ccc4cc(-n5c(CC)nc6ccccc65)ccc4c3)c3c([2H])c([2H])c([2H])c([2H])c23)c([2H])c1[2H]]

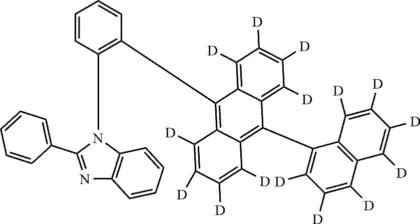 [SMILES: [2H]c1c([2H])c([2H])c2c(-c3c4c([2H])c([2H])c([2H])c([2H])c4c(-c4ccccc4-n4c(-c5ccccc5)nc5ccccc54)c4c([2H])c([2H])c([2H])c([2H])c34)c([2H])c([2H])c([2H])c2c1[2H]]

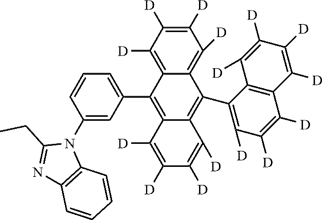 [SMILES: [2H]c1c([2H])c([2H])c2c(-c3c4c([2H])c([2H])c([2H])c([2H])c4c(-c4cccc(-n5c(CC)nc6ccccc65)c4)c4c([2H])c([2H])c([2H])c([2H])c34)c([2H])c([2H])c([2H])c2c1[2H]]

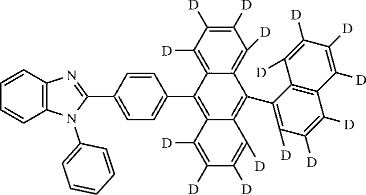 [SMILES: [2H]c1c([2H])c([2H])c2c(-c3c4c([2H])c([2H])c([2H])c([2H])c4c(-c4ccc(-c5nc6ccccc6n5-c5ccccc5)cc4)c4c([2H])c([2H])c([2H])c([2H])c34)c([2H])c([2H])c([2H])c2c1[2H]]

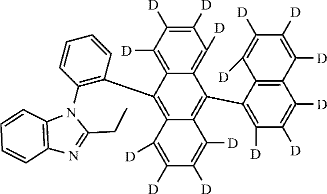 [SMILES: [2H]c1c([2H])c([2H])c2c(-c3c4c([2H])c([2H])c([2H])c([2H])c4c(-c4ccccc4-n4c(CC)nc5ccccc54)c4c([2H])c([2H])c([2H])c([2H])c34)c([2H])c([2H])c([2H])c2c1[2H]]

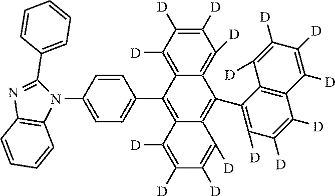 [SMILES: [2H]c1c([2H])c([2H])c2c(-c3c4c([2H])c([2H])c([2H])c([2H])c4c(-c4ccc(-n5c(-c6ccccc6)nc6ccccc65)cc4)c4c([2H])c([2H])c([2H])c([2H])c34)c([2H])c([2H])c([2H])c2c1[2H]]

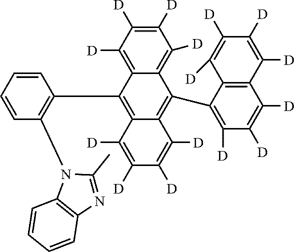 [SMILES: [2H]c1c([2H])c([2H])c2c(-c3c4c([2H])c([2H])c([2H])c([2H])c4c(-c4ccccc4-n4c(C)nc5ccccc54)c4c([2H])c([2H])c([2H])c([2H])c34)c([2H])c([2H])c([2H])c2c1[2H]]

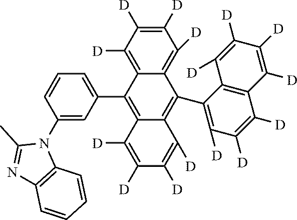 [SMILES: [2H]c1c([2H])c([2H])c2c(-c3c4c([2H])c([2H])c([2H])c([2H])c4c(-c4cccc(-n5c(C)nc6ccccc65)c4)c4c([2H])c([2H])c([2H])c([2H])c34)c([2H])c([2H])c([2H])c2c1[2H]]

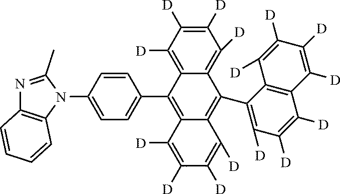 [SMILES: [2H]c1c([2H])c([2H])c2c(-c3c4c([2H])c([2H])c([2H])c([2H])c4c(-c4ccc(-n5c(C)nc6ccccc65)cc4)c4c([2H])c([2H])c([2H])c([2H])c34)c([2H])c([2H])c([2H])c2c1[2H]]

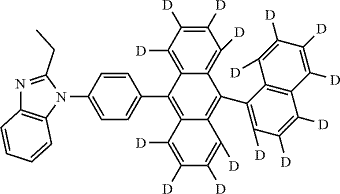 [SMILES: [2H]c1c([2H])c([2H])c2c(-c3c4c([2H])c([2H])c([2H])c([2H])c4c(-c4ccc(-n5c(CC)nc6ccccc65)cc4)c4c([2H])c([2H])c([2H])c([2H])c34)c([2H])c([2H])c([2H])c2c1[2H]]

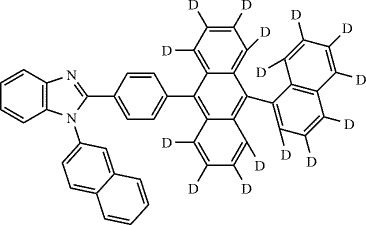 [SMILES: [2H]c1c([2H])c([2H])c2c(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)c([2H])c([2H])c([2H])c2c1[2H]]

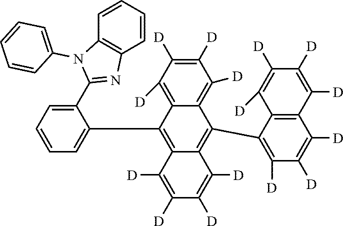 [SMILES: [2H]c1c([2H])c([2H])c2c(-c3c4c([2H])c([2H])c([2H])c([2H])c4c(-c4ccccc4-c4nc5ccccc5n4-c4ccccc4)c4c([2H])c([2H])c([2H])c([2H])c34)c([2H])c([2H])c([2H])c2c1[2H]]

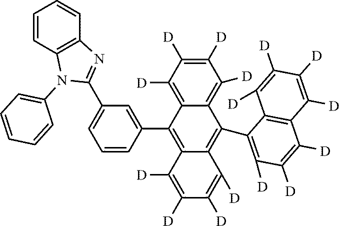 [SMILES: [2H]c1c([2H])c([2H])c2c(-c3c4c([2H])c([2H])c([2H])c([2H])c4c(-c4cccc(-c5nc6ccccc6n5-c5ccccc5)c4)c4c([2H])c([2H])c([2H])c([2H])c34)c([2H])c([2H])c([2H])c2c1[2H]]

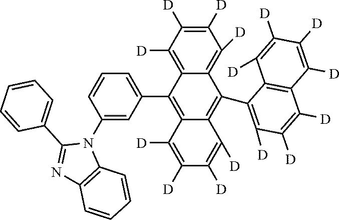 [SMILES: [2H]c1c([2H])c([2H])c2c(-c3c4c([2H])c([2H])c([2H])c([2H])c4c(-c4cccc(-n5c(-c6ccccc6)nc6ccccc65)c4)c4c([2H])c([2H])c([2H])c([2H])c34)c([2H])c([2H])c([2H])c2c1[2H]]

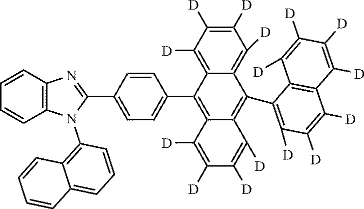 [SMILES: [2H]c1c([2H])c([2H])c2c(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)c([2H])c([2H])c([2H])c2c1[2H]]

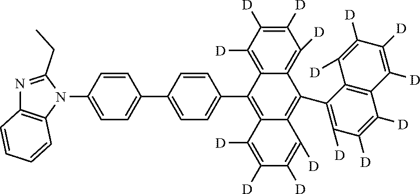 [SMILES: [2H]c1c([2H])c([2H])c2c(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)c([2H])c([2H])c([2H])c2c1[2H]]

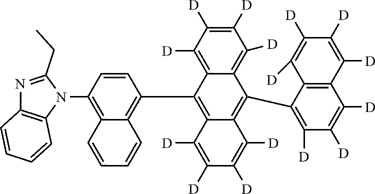 [SMILES: [2H]c1c([2H])c([2H])c2c(-c3c4c([2H])c([2H])c([2H])c([2H])c4c(-c4ccc(-n5c(CC)nc6ccccc65)c5ccccc45)c4c([2H])c([2H])c([2H])c([2H])c34)c([2H])c([2H])c([2H])c2c1[2H]]

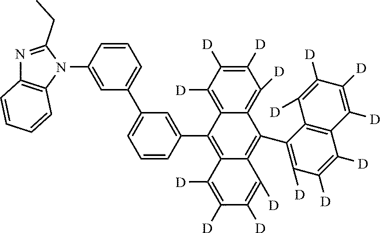 [SMILES: [2H]c1c([2H])c([2H])c2c(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)c([2H])c([2H])c([2H])c2c1[2H]]

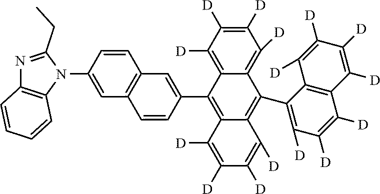 [SMILES: [2H]c1c([2H])c([2H])c2c(-c3c4c([2H])c([2H])c([2H])c([2H])c4c(-c4ccc5cc(-n6c(CC)nc7ccccc76)ccc5c4)c4c([2H])c([2H])c([2H])c([2H])c34)c([2H])c([2H])c([2H])c2c1[2H]]

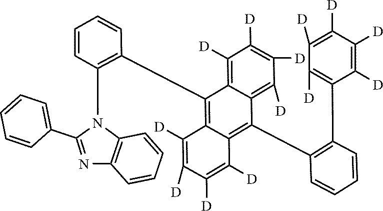 [SMILES: [2H]c1c([2H])c([2H])c(-c2ccccc2-c2c3c([2H])c([2H])c([2H])c([2H])c3c(-c3ccccc3-n3c(-c4ccccc4)nc4ccccc43)c3c([2H])c([2H])c([2H])c([2H])c23)c([2H])c1[2H]]

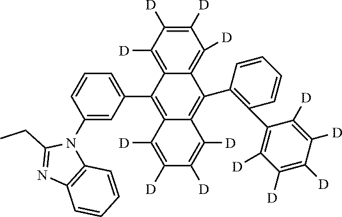 [SMILES: [2H]c1c([2H])c([2H])c(-c2ccccc2-c2c3c([2H])c([2H])c([2H])c([2H])c3c(-c3cccc(-n4c(CC)nc5ccccc54)c3)c3c([2H])c([2H])c([2H])c([2H])c23)c([2H])c1[2H]]

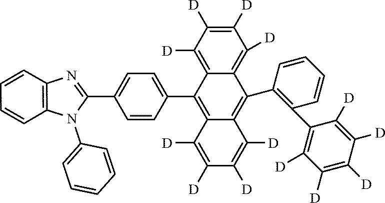 [SMILES: [2H]c1c([2H])c([2H])c(-c2ccccc2-c2c3c([2H])c([2H])c([2H])c([2H])c3c(-c3ccc(-c4nc5ccccc5n4-c4ccccc4)cc3)c3c([2H])c([2H])c([2H])c([2H])c23)c([2H])c1[2H]]

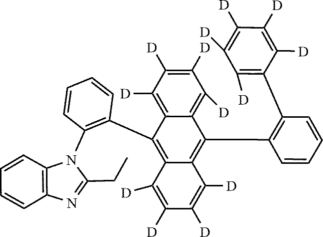 [SMILES: [2H]c1c([2H])c([2H])c(-c2ccccc2-c2c3c([2H])c([2H])c([2H])c([2H])c3c(-c3ccccc3-n3c(CC)nc4ccccc43)c3c([2H])c([2H])c([2H])c([2H])c23)c([2H])c1[2H]]

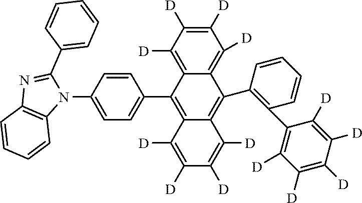 [SMILES: [2H]c1c([2H])c([2H])c(-c2ccccc2-c2c3c([2H])c([2H])c([2H])c([2H])c3c(-c3ccc(-n4c(-c5ccccc5)nc5ccccc54)cc3)c3c([2H])c([2H])c([2H])c([2H])c23)c([2H])c1[2H]]

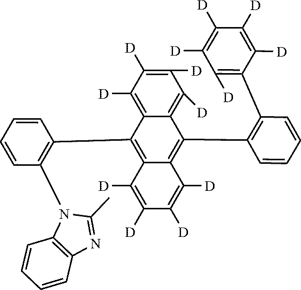 [SMILES: [2H]c1c([2H])c([2H])c(-c2ccccc2-c2c3c([2H])c([2H])c([2H])c([2H])c3c(-c3ccccc3-n3c(C)nc4ccccc43)c3c([2H])c([2H])c([2H])c([2H])c23)c([2H])c1[2H]]

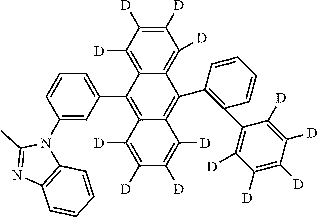 [SMILES: [2H]c1c([2H])c([2H])c(-c2ccccc2-c2c3c([2H])c([2H])c([2H])c([2H])c3c(-c3cccc(-n4c(C)nc5ccccc54)c3)c3c([2H])c([2H])c([2H])c([2H])c23)c([2H])c1[2H]]

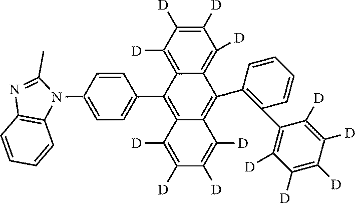 [SMILES: [2H]c1c([2H])c([2H])c(-c2ccccc2-c2c3c([2H])c([2H])c([2H])c([2H])c3c(-c3ccc(-n4c(C)nc5ccccc54)cc3)c3c([2H])c([2H])c([2H])c([2H])c23)c([2H])c1[2H]]

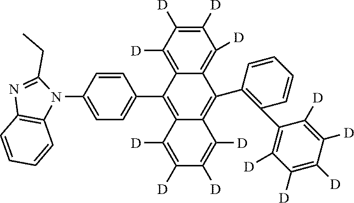 [SMILES: [2H]c1c([2H])c([2H])c(-c2ccccc2-c2c3c([2H])c([2H])c([2H])c([2H])c3c(-c3ccc(-n4c(CC)nc5ccccc54)cc3)c3c([2H])c([2H])c([2H])c([2H])c23)c([2H])c1[2H]]

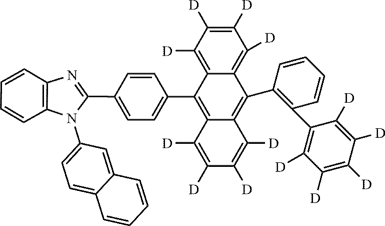 [SMILES: [2H]c1c([2H])c([2H])c(-c2ccccc2-c2c3c([2H])c([2H])c([2H])c([2H])c3c(-c3ccc(-c4nc5ccccc5n4-c4ccc5ccccc5c4)cc3)c3c([2H])c([2H])c([2H])c([2H])c23)c([2H])c1[2H]]

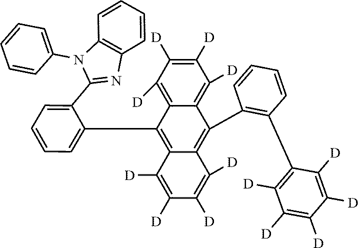 [SMILES: [2H]c1c([2H])c([2H])c(-c2ccccc2-c2c3c([2H])c([2H])c([2H])c([2H])c3c(-c3ccccc3-c3nc4ccccc4n3-c3ccccc3)c3c([2H])c([2H])c([2H])c([2H])c23)c([2H])c1[2H]]

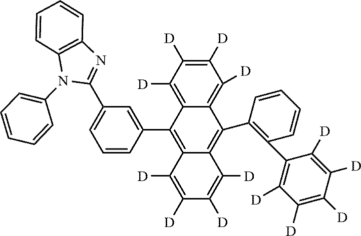 [SMILES: [2H]c1c([2H])c([2H])c(-c2ccccc2-c2c3c([2H])c([2H])c([2H])c([2H])c3c(-c3cccc(-c4nc5ccccc5n4-c4ccccc4)c3)c3c([2H])c([2H])c([2H])c([2H])c23)c([2H])c1[2H]]

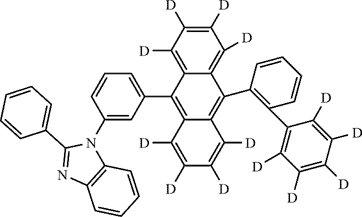 [SMILES: [2H]c1c([2H])c([2H])c(-c2ccccc2-c2c3c([2H])c([2H])c([2H])c([2H])c3c(-c3cccc(-n4c(-c5ccccc5)nc5ccccc54)c3)c3c([2H])c([2H])c([2H])c([2H])c23)c([2H])c1[2H]]

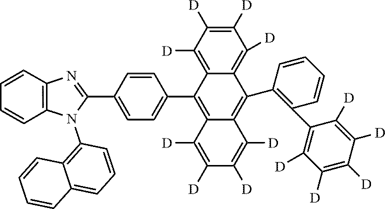 [SMILES: [2H]c1c([2H])c([2H])c(-c2ccccc2-c2c3c([2H])c([2H])c([2H])c([2H])c3c(-c3ccc(-c4nc5ccccc5n4-c4cccc5ccccc45)cc3)c3c([2H])c([2H])c([2H])c([2H])c23)c([2H])c1[2H]]

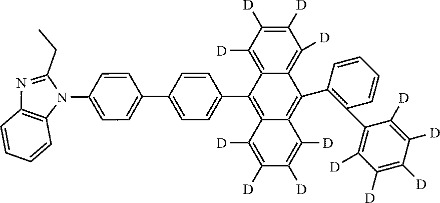 [SMILES: [2H]c1c([2H])c([2H])c(-c2ccccc2-c2c3c([2H])c([2H])c([2H])c([2H])c3c(-c3ccc(-c4ccc(-n5c(CC)nc6ccccc65)cc4)cc3)c3c([2H])c([2H])c([2H])c([2H])c23)c([2H])c1[2H]]

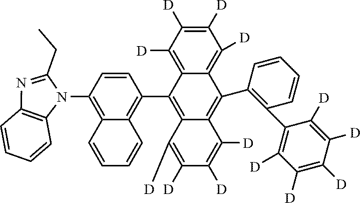 [SMILES: [2H]c1c([2H])c([2H])c(-c2ccccc2-c2c3c([2H])c([2H])c([2H])c([2H])c3c(-c3ccc(-n4c(CC)nc5ccccc54)c4ccccc34)c3c([2H])c([2H])c([2H])c([2H])c23)c([2H])c1[2H]]

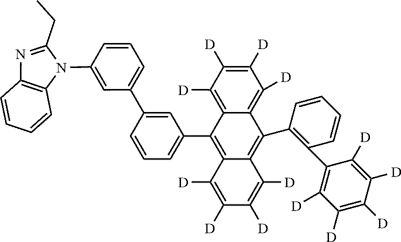 [SMILES: [2H]c1c([2H])c([2H])c(-c2ccccc2-c2c3c([2H])c([2H])c([2H])c([2H])c3c(-c3cccc(-c4cccc(-n5c(CC)nc6ccccc65)c4)c3)c3c([2H])c([2H])c([2H])c([2H])c23)c([2H])c1[2H]]

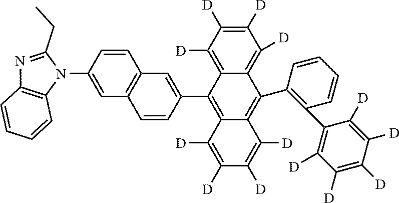 [SMILES: [2H]c1c([2H])c([2H])c(-c2ccccc2-c2c3c([2H])c([2H])c([2H])c([2H])c3c(-c3ccc4cc(-n5c(CC)nc6ccccc65)ccc4c3)c3c([2H])c([2H])c([2H])c([2H])c23)c([2H])c1[2H]]

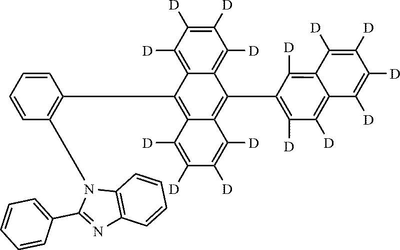 [SMILES: [2H]c1c([2H])c([2H])c2c([2H])c(-c3c4c([2H])c([2H])c([2H])c([2H])c4c(-c4ccccc4-n4c(-c5ccccc5)nc5ccccc54)c4c([2H])c([2H])c([2H])c([2H])c34)c([2H])c([2H])c2c1[2H]]

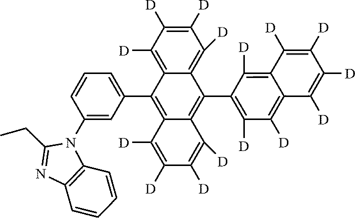 [SMILES: [2H]c1c([2H])c([2H])c2c([2H])c(-c3c4c([2H])c([2H])c([2H])c([2H])c4c(-c4cccc(-n5c(CC)nc6ccccc65)c4)c4c([2H])c([2H])c([2H])c([2H])c34)c([2H])c([2H])c2c1[2H]]

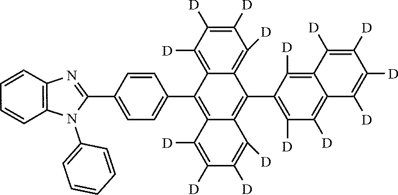 [SMILES: [2H]c1c([2H])c([2H])c2c([2H])c(-c3c4c([2H])c([2H])c([2H])c([2H])c4c(-c4ccc(-c5nc6ccccc6n5-c5ccccc5)cc4)c4c([2H])c([2H])c([2H])c([2H])c34)c([2H])c([2H])c2c1[2H]]

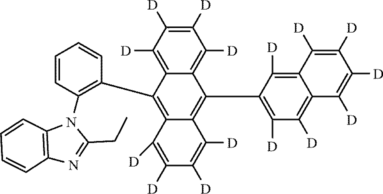 [SMILES: [2H]c1c([2H])c([2H])c2c([2H])c(-c3c4c([2H])c([2H])c([2H])c([2H])c4c(-c4ccccc4-n4c(CC)nc5ccccc54)c4c([2H])c([2H])c([2H])c([2H])c34)c([2H])c([2H])c2c1[2H]]

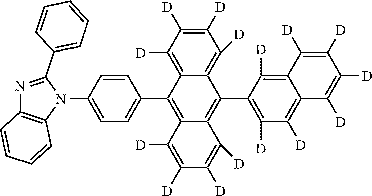 [SMILES: [2H]c1c([2H])c([2H])c2c([2H])c(-c3c4c([2H])c([2H])c([2H])c([2H])c4c(-c4ccc(-n5c(-c6ccccc6)nc6ccccc65)cc4)c4c([2H])c([2H])c([2H])c([2H])c34)c([2H])c([2H])c2c1[2H]]

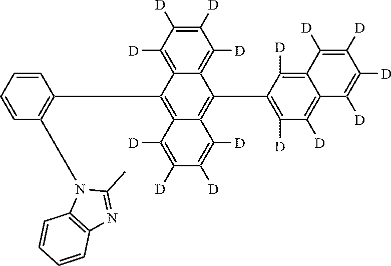 [SMILES: [2H]c1c([2H])c([2H])c2c([2H])c(-c3c4c([2H])c([2H])c([2H])c([2H])c4c(-c4ccccc4-n4c(C)nc5ccccc54)c4c([2H])c([2H])c([2H])c([2H])c34)c([2H])c([2H])c2c1[2H]]

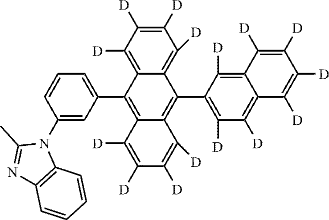 [SMILES: [2H]c1c([2H])c([2H])c2c([2H])c(-c3c4c([2H])c([2H])c([2H])c([2H])c4c(-c4cccc(-n5c(C)nc6ccccc65)c4)c4c([2H])c([2H])c([2H])c([2H])c34)c([2H])c([2H])c2c1[2H]]

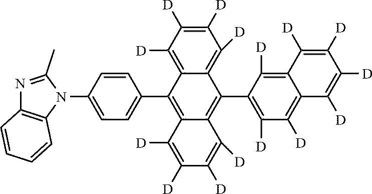 [SMILES: [2H]c1c([2H])c([2H])c2c([2H])c(-c3c4c([2H])c([2H])c([2H])c([2H])c4c(-c4ccc(-n5c(C)nc6ccccc65)cc4)c4c([2H])c([2H])c([2H])c([2H])c34)c([2H])c([2H])c2c1[2H]]

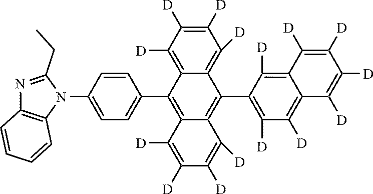 [SMILES: [2H]c1c([2H])c([2H])c2c([2H])c(-c3c4c([2H])c([2H])c([2H])c([2H])c4c(-c4ccc(-n5c(CC)nc6ccccc65)cc4)c4c([2H])c([2H])c([2H])c([2H])c34)c([2H])c([2H])c2c1[2H]]

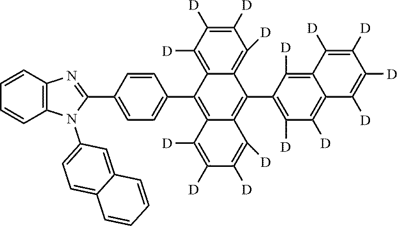 [SMILES: [2H]c1c([2H])c([2H])c2c([2H])c(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)c([2H])c([2H])c2c1[2H]]

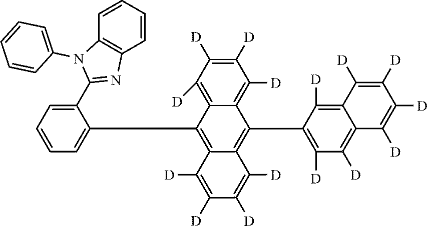 [SMILES: [2H]c1c([2H])c([2H])c2c([2H])c(-c3c4c([2H])c([2H])c([2H])c([2H])c4c(-c4ccccc4-c4nc5ccccc5n4-c4ccccc4)c4c([2H])c([2H])c([2H])c([2H])c34)c([2H])c([2H])c2c1[2H]]

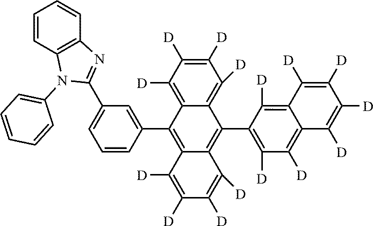 [SMILES: [2H]c1c([2H])c([2H])c2c([2H])c(-c3c4c([2H])c([2H])c([2H])c([2H])c4c(-c4cccc(-c5nc6ccccc6n5-c5ccccc5)c4)c4c([2H])c([2H])c([2H])c([2H])c34)c([2H])c([2H])c2c1[2H]]

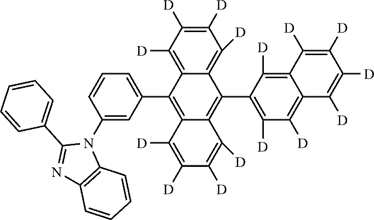 [SMILES: [2H]c1c([2H])c([2H])c2c([2H])c(-c3c4c([2H])c([2H])c([2H])c([2H])c4c(-c4cccc(-n5c(-c6ccccc6)nc6ccccc65)c4)c4c([2H])c([2H])c([2H])c([2H])c34)c([2H])c([2H])c2c1[2H]]

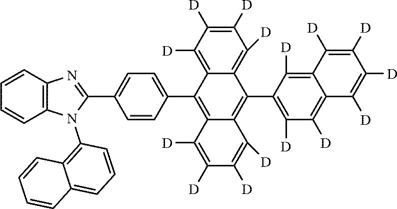 [SMILES: [2H]c1c([2H])c([2H])c2c([2H])c(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)c([2H])c([2H])c2c1[2H]]

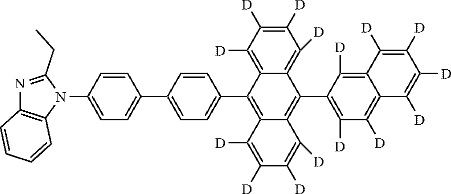 [SMILES: [2H]c1c([2H])c([2H])c2c([2H])c(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)c([2H])c([2H])c2c1[2H]]

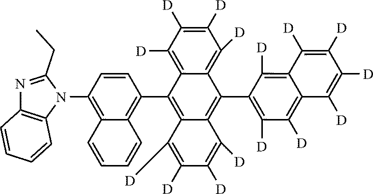 [SMILES: [2H]c1c([2H])c([2H])c2c([2H])c(-c3c4c([2H])c([2H])c([2H])c([2H])c4c(-c4ccc(-n5c(CC)nc6ccccc65)c5ccccc45)c4c([2H])c([2H])c([2H])c([2H])c34)c([2H])c([2H])c2c1[2H]]

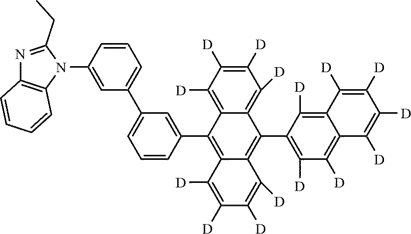 [SMILES: [2H]c1c([2H])c([2H])c2c([2H])c(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)c([2H])c([2H])c2c1[2H]]

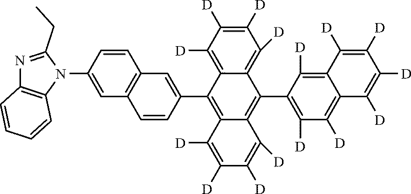 [SMILES: [2H]c1c([2H])c([2H])c2c([2H])c(-c3c4c([2H])c([2H])c([2H])c([2H])c4c(-c4ccc5cc(-n6c(CC)nc7ccccc76)ccc5c4)c4c([2H])c([2H])c([2H])c([2H])c34)c([2H])c([2H])c2c1[2H]]

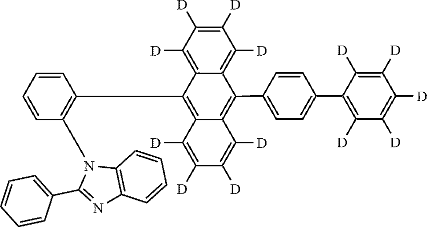 [SMILES: [2H]c1c([2H])c([2H])c(-c2ccc(-c3c4c([2H])c([2H])c([2H])c([2H])c4c(-c4ccccc4-n4c(-c5ccccc5)nc5ccccc54)c4c([2H])c([2H])c([2H])c([2H])c34)cc2)c([2H])c1[2H]]

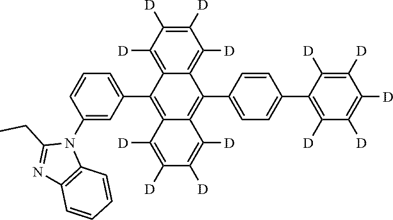 [SMILES: [2H]c1c([2H])c([2H])c(-c2ccc(-c3c4c([2H])c([2H])c([2H])c([2H])c4c(-c4cccc(-n5c(CC)nc6ccccc65)c4)c4c([2H])c([2H])c([2H])c([2H])c34)cc2)c([2H])c1[2H]]

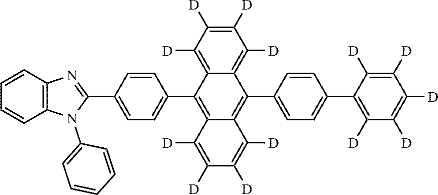 [SMILES: [2H]c1c([2H])c([2H])c(-c2ccc(-c3c4c([2H])c([2H])c([2H])c([2H])c4c(-c4ccc(-c5nc6ccccc6n5-c5ccccc5)cc4)c4c([2H])c([2H])c([2H])c([2H])c34)cc2)c([2H])c1[2H]]

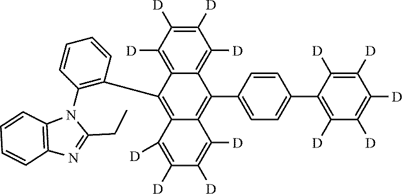 [SMILES: [2H]c1c([2H])c([2H])c(-c2ccc(-c3c4c([2H])c([2H])c([2H])c([2H])c4c(-c4ccccc4-n4c(CC)nc5ccccc54)c4c([2H])c([2H])c([2H])c([2H])c34)cc2)c([2H])c1[2H]]

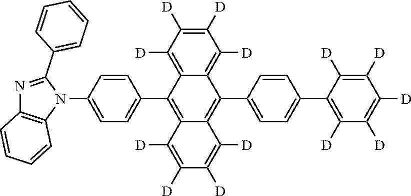 [SMILES: [2H]c1c([2H])c([2H])c(-c2ccc(-c3c4c([2H])c([2H])c([2H])c([2H])c4c(-c4ccc(-n5c(-c6ccccc6)nc6ccccc65)cc4)c4c([2H])c([2H])c([2H])c([2H])c34)cc2)c([2H])c1[2H]]

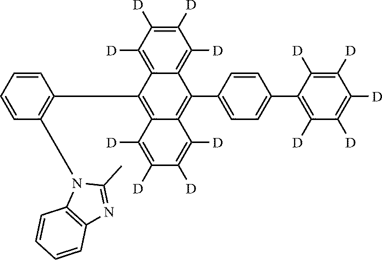 [SMILES: [2H]c1c([2H])c([2H])c(-c2ccc(-c3c4c([2H])c([2H])c([2H])c([2H])c4c(-c4ccccc4-n4c(C)nc5ccccc54)c4c([2H])c([2H])c([2H])c([2H])c34)cc2)c([2H])c1[2H]]

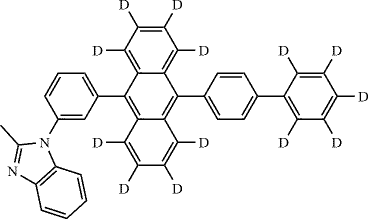 [SMILES: [2H]c1c([2H])c([2H])c(-c2ccc(-c3c4c([2H])c([2H])c([2H])c([2H])c4c(-c4cccc(-n5c(C)nc6ccccc65)c4)c4c([2H])c([2H])c([2H])c([2H])c34)cc2)c([2H])c1[2H]]

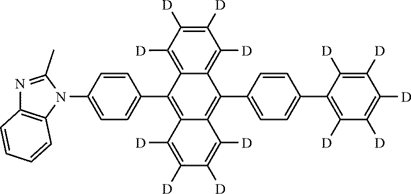 [SMILES: [2H]c1c([2H])c([2H])c(-c2ccc(-c3c4c([2H])c([2H])c([2H])c([2H])c4c(-c4ccc(-n5c(C)nc6ccccc65)cc4)c4c([2H])c([2H])c([2H])c([2H])c34)cc2)c([2H])c1[2H]]

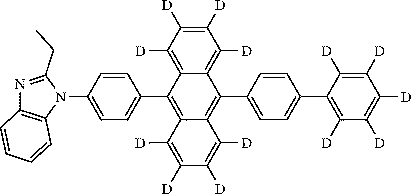 [SMILES: [2H]c1c([2H])c([2H])c(-c2ccc(-c3c4c([2H])c([2H])c([2H])c([2H])c4c(-c4ccc(-n5c(CC)nc6ccccc65)cc4)c4c([2H])c([2H])c([2H])c([2H])c34)cc2)c([2H])c1[2H]]

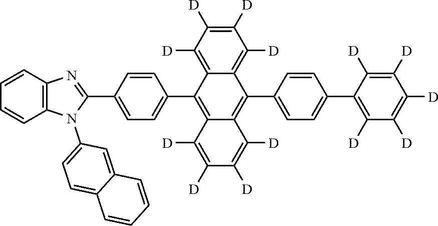 [SMILES: [2H]c1c([2H])c([2H])c(-c2ccc(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)cc2)c([2H])c1[2H]]

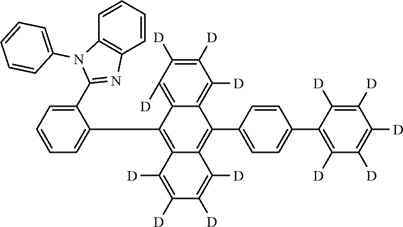 [SMILES: [2H]c1c([2H])c([2H])c(-c2ccc(-c3c4c([2H])c([2H])c([2H])c([2H])c4c(-c4ccccc4-c4nc5ccccc5n4-c4ccccc4)c4c([2H])c([2H])c([2H])c([2H])c34)cc2)c([2H])c1[2H]]

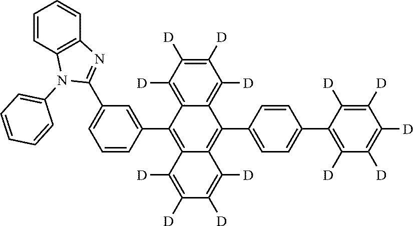 [SMILES: [2H]c1c([2H])c([2H])c(-c2ccc(-c3c4c([2H])c([2H])c([2H])c([2H])c4c(-c4cccc(-c5nc6ccccc6n5-c5ccccc5)c4)c4c([2H])c([2H])c([2H])c([2H])c34)cc2)c([2H])c1[2H]]

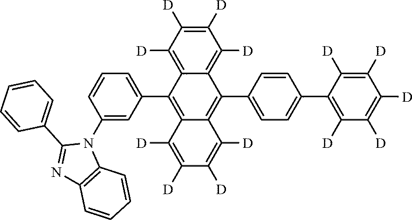 [SMILES: [2H]c1c([2H])c([2H])c(-c2ccc(-c3c4c([2H])c([2H])c([2H])c([2H])c4c(-c4cccc(-n5c(-c6ccccc6)nc6ccccc65)c4)c4c([2H])c([2H])c([2H])c([2H])c34)cc2)c([2H])c1[2H]]

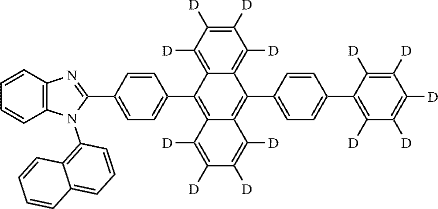 [SMILES: [2H]c1c([2H])c([2H])c(-c2ccc(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)cc2)c([2H])c1[2H]]

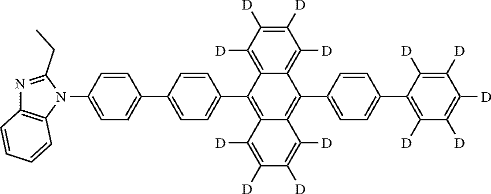 [SMILES: [2H]c1c([2H])c([2H])c(-c2ccc(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)cc2)c([2H])c1[2H]]

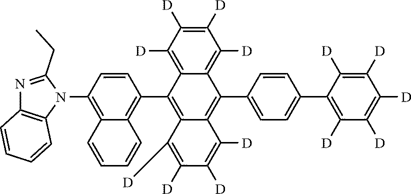 [SMILES: [2H]c1c([2H])c([2H])c(-c2ccc(-c3c4c([2H])c([2H])c([2H])c([2H])c4c(-c4ccc(-n5c(CC)nc6ccccc65)c5ccccc45)c4c([2H])c([2H])c([2H])c([2H])c34)cc2)c([2H])c1[2H]]

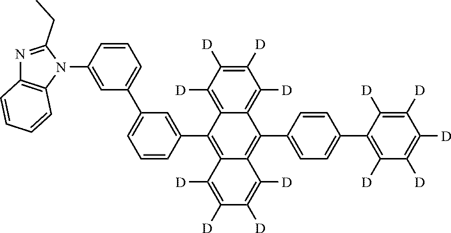 [SMILES: [2H]c1c([2H])c([2H])c(-c2ccc(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)cc2)c([2H])c1[2H]]

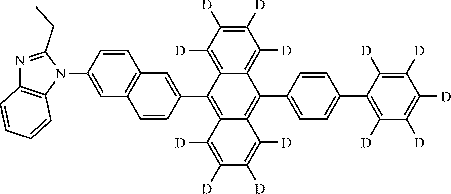 [SMILES: [2H]c1c([2H])c([2H])c(-c2ccc(-c3c4c([2H])c([2H])c([2H])c([2H])c4c(-c4ccc5cc(-n6c(CC)nc7ccccc76)ccc5c4)c4c([2H])c([2H])c([2H])c([2H])c34)cc2)c([2H])c1[2H]]

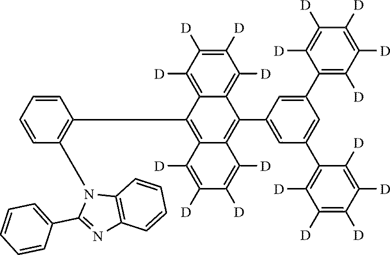 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(-c5ccccc5)nc5ccccc54)c4c([2H])c([2H])c([2H])c([2H])c34)c2)c([2H])c1[2H]]

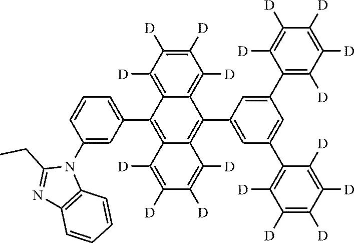 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(CC)nc6ccccc65)c4)c4c([2H])c([2H])c([2H])c([2H])c34)c2)c([2H])c1[2H]]

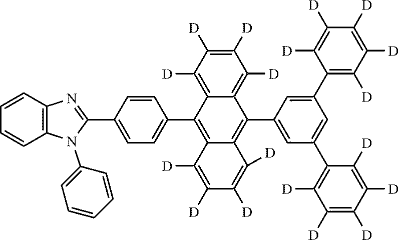 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5nc6ccccc6n5-c5ccccc5)cc4)c4c([2H])c([2H])c([2H])c([2H])c34)c2)c([2H])c1[2H]]

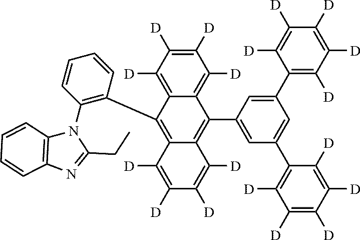 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(CC)nc5ccccc54)c4c([2H])c([2H])c([2H])c([2H])c34)c2)c([2H])c1[2H]]

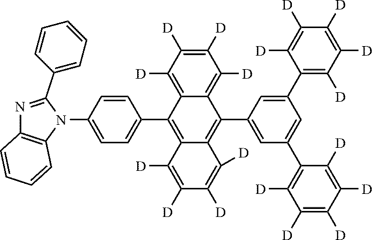 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(-c6ccccc6)nc6ccccc65)cc4)c4c([2H])c([2H])c([2H])c([2H])c34)c2)c([2H])c1[2H]]

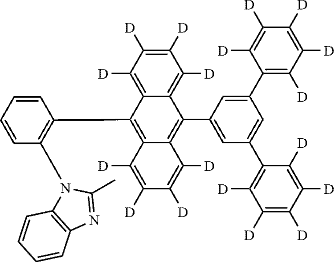 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-n4c(C)nc5ccccc54)c4c([2H])c([2H])c([2H])c([2H])c34)c2)c([2H])c1[2H]]

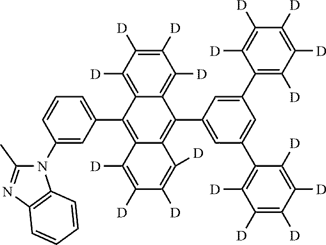 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(C)nc6ccccc65)c4)c4c([2H])c([2H])c([2H])c([2H])c34)c2)c([2H])c1[2H]]

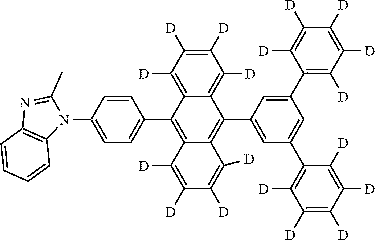 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(C)nc6ccccc65)cc4)c4c([2H])c([2H])c([2H])c([2H])c34)c2)c([2H])c1[2H]]

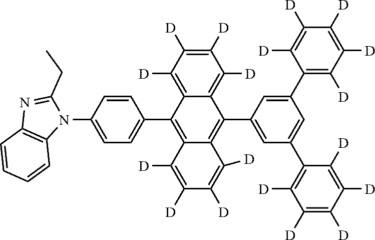 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(CC)nc6ccccc65)cc4)c4c([2H])c([2H])c([2H])c([2H])c34)c2)c([2H])c1[2H]]

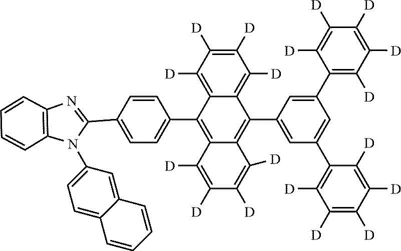 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)c2)c([2H])c1[2H]]

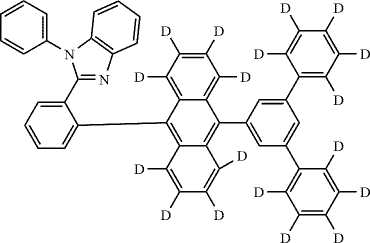 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccccc4-c4nc5ccccc5n4-c4ccccc4)c4c([2H])c([2H])c([2H])c([2H])c34)c2)c([2H])c1[2H]]

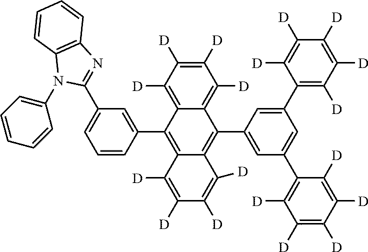 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-c5nc6ccccc6n5-c5ccccc5)c4)c4c([2H])c([2H])c([2H])c([2H])c34)c2)c([2H])c1[2H]]

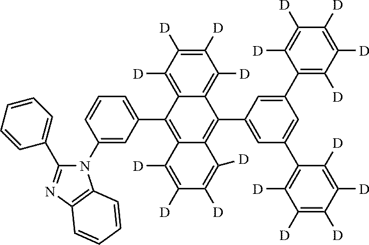 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-n5c(-c6ccccc6)nc6ccccc65)c4)c4c([2H])c([2H])c([2H])c([2H])c34)c2)c([2H])c1[2H]]

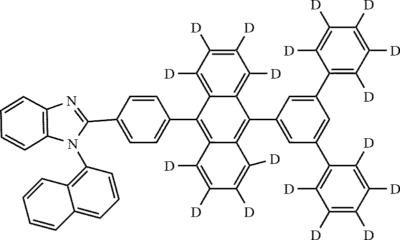 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)c2)c([2H])c1[2H]]

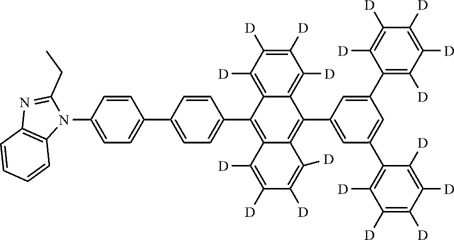 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)c2)c([2H])c1[2H]]

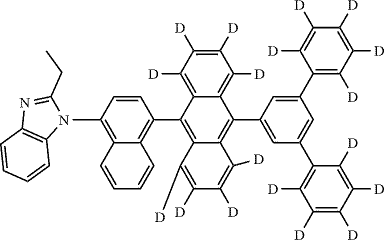 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc(-n5c(CC)nc6ccccc65)c5ccccc45)c4c([2H])c([2H])c([2H])c([2H])c34)c2)c([2H])c1[2H]]

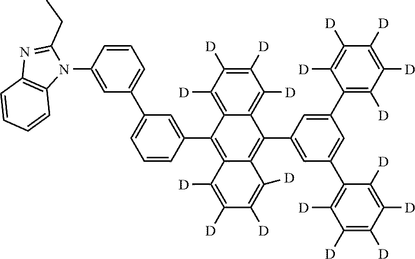 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)c2)c([2H])c1[2H]]

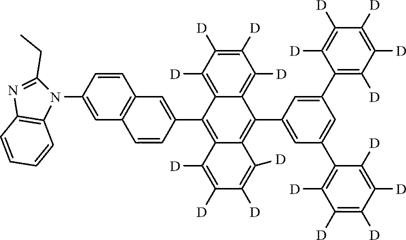 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4ccc5cc(-n6c(CC)nc7ccccc76)ccc5c4)c4c([2H])c([2H])c([2H])c([2H])c34)c2)c([2H])c1[2H]]

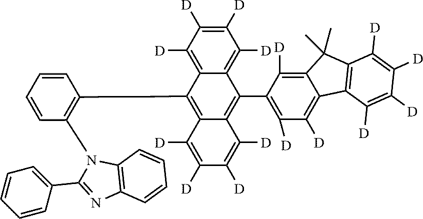 [SMILES: [2H]c1c([2H])c([2H])c2c(c1[2H])-c1c([2H])c([2H])c(-c3c4c([2H])c([2H])c([2H])c([2H])c4c(-c4ccccc4-n4c(-c5ccccc5)nc5ccccc54)c4c([2H])c([2H])c([2H])c([2H])c34)c([2H])c1C2(C)C]

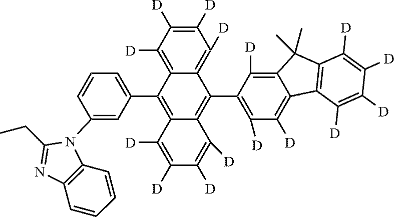 [SMILES: [2H]c1c([2H])c([2H])c2c(c1[2H])-c1c([2H])c([2H])c(-c3c4c([2H])c([2H])c([2H])c([2H])c4c(-c4cccc(-n5c(CC)nc6ccccc65)c4)c4c([2H])c([2H])c([2H])c([2H])c34)c([2H])c1C2(C)C]

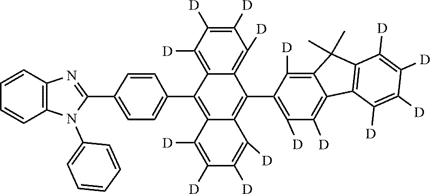 [SMILES: [2H]c1c([2H])c([2H])c2c(c1[2H])-c1c([2H])c([2H])c(-c3c4c([2H])c([2H])c([2H])c([2H])c4c(-c4ccc(-c5nc6ccccc6n5-c5ccccc5)cc4)c4c([2H])c([2H])c([2H])c([2H])c34)c([2H])c1C2(C)C]

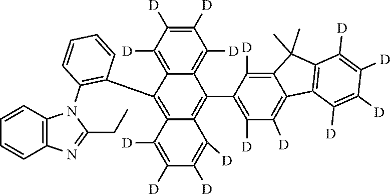 [SMILES: [2H]c1c([2H])c([2H])c2c(c1[2H])-c1c([2H])c([2H])c(-c3c4c([2H])c([2H])c([2H])c([2H])c4c(-c4ccccc4-n4c(CC)nc5ccccc54)c4c([2H])c([2H])c([2H])c([2H])c34)c([2H])c1C2(C)C]

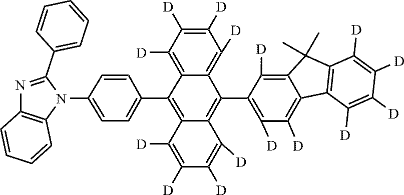 [SMILES: [2H]c1c([2H])c([2H])c2c(c1[2H])-c1c([2H])c([2H])c(-c3c4c([2H])c([2H])c([2H])c([2H])c4c(-c4ccc(-n5c(-c6ccccc6)nc6ccccc65)cc4)c4c([2H])c([2H])c([2H])c([2H])c34)c([2H])c1C2(C)C]

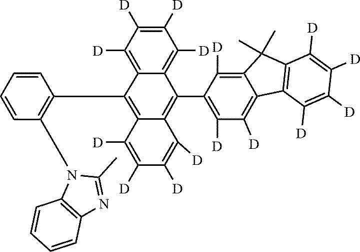 [SMILES: [2H]c1c([2H])c([2H])c2c(c1[2H])-c1c([2H])c([2H])c(-c3c4c([2H])c([2H])c([2H])c([2H])c4c(-c4ccccc4-n4c(C)nc5ccccc54)c4c([2H])c([2H])c([2H])c([2H])c34)c([2H])c1C2(C)C]

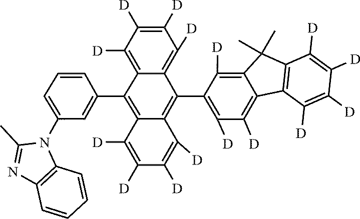 [SMILES: [2H]c1c([2H])c([2H])c2c(c1[2H])-c1c([2H])c([2H])c(-c3c4c([2H])c([2H])c([2H])c([2H])c4c(-c4cccc(-n5c(C)nc6ccccc65)c4)c4c([2H])c([2H])c([2H])c([2H])c34)c([2H])c1C2(C)C]

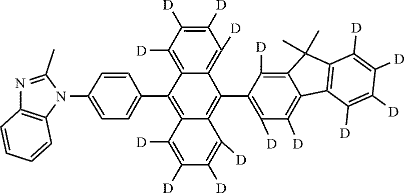 [SMILES: [2H]c1c([2H])c([2H])c2c(c1[2H])-c1c([2H])c([2H])c(-c3c4c([2H])c([2H])c([2H])c([2H])c4c(-c4ccc(-n5c(C)nc6ccccc65)cc4)c4c([2H])c([2H])c([2H])c([2H])c34)c([2H])c1C2(C)C]

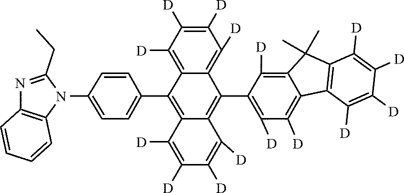 [SMILES: [2H]c1c([2H])c([2H])c2c(c1[2H])-c1c([2H])c([2H])c(-c3c4c([2H])c([2H])c([2H])c([2H])c4c(-c4ccc(-n5c(CC)nc6ccccc65)cc4)c4c([2H])c([2H])c([2H])c([2H])c34)c([2H])c1C2(C)C]

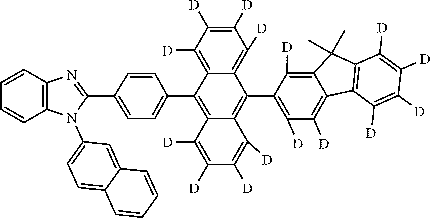 [SMILES: [2H]c1c([2H])c([2H])c2c(c1[2H])-c1c([2H])c([2H])c(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)c([2H])c1C2(C)C]

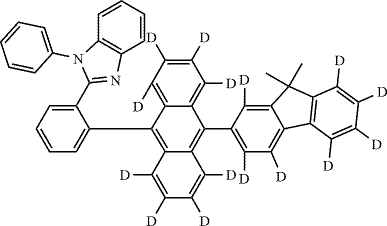 [SMILES: [2H]c1c([2H])c([2H])c2c(c1[2H])-c1c([2H])c([2H])c(-c3c4c([2H])c([2H])c([2H])c([2H])c4c(-c4ccccc4-c4nc5ccccc5n4-c4ccccc4)c4c([2H])c([2H])c([2H])c([2H])c34)c([2H])c1C2(C)C]

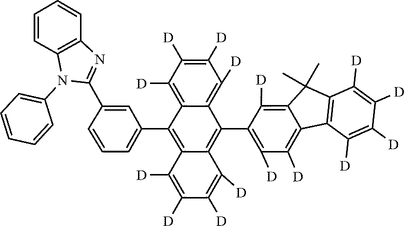 [SMILES: [2H]c1c([2H])c([2H])c2c(c1[2H])-c1c([2H])c([2H])c(-c3c4c([2H])c([2H])c([2H])c([2H])c4c(-c4cccc(-c5nc6ccccc6n5-c5ccccc5)c4)c4c([2H])c([2H])c([2H])c([2H])c34)c([2H])c1C2(C)C]

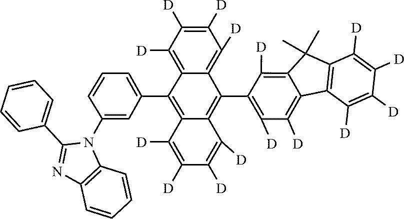 [SMILES: [2H]c1c([2H])c([2H])c2c(c1[2H])-c1c([2H])c([2H])c(-c3c4c([2H])c([2H])c([2H])c([2H])c4c(-c4cccc(-n5c(-c6ccccc6)nc6ccccc65)c4)c4c([2H])c([2H])c([2H])c([2H])c34)c([2H])c1C2(C)C]

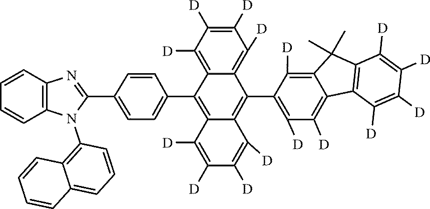 [SMILES: [2H]c1c([2H])c([2H])c2c(c1[2H])-c1c([2H])c([2H])c(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)c([2H])c1C2(C)C]

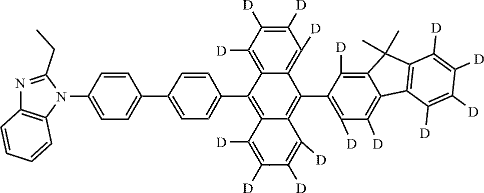 [SMILES: [2H]c1c([2H])c([2H])c2c(c1[2H])-c1c([2H])c([2H])c(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)c([2H])c1C2(C)C]

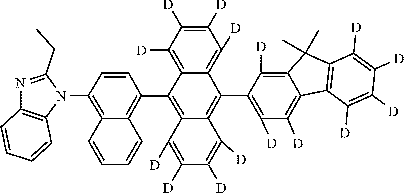 [SMILES: [2H]c1c([2H])c([2H])c2c(c1[2H])-c1c([2H])c([2H])c(-c3c4c([2H])c([2H])c([2H])c([2H])c4c(-c4ccc(-n5c(CC)nc6ccccc65)c5ccccc45)c4c([2H])c([2H])c([2H])c([2H])c34)c([2H])c1C2(C)C]

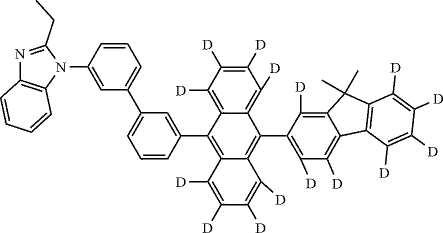 [SMILES: [2H]c1c([2H])c([2H])c2c(c1[2H])-c1c([2H])c([2H])c(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)c([2H])c1C2(C)C]

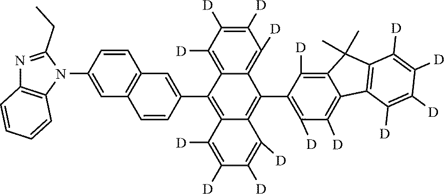 [SMILES: [2H]c1c([2H])c([2H])c2c(c1[2H])-c1c([2H])c([2H])c(-c3c4c([2H])c([2H])c([2H])c([2H])c4c(-c4ccc5cc(-n6c(CC)nc7ccccc76)ccc5c4)c4c([2H])c([2H])c([2H])c([2H])c34)c([2H])c1C2(C)C]

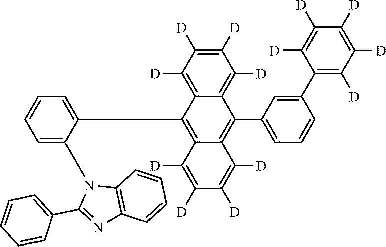 [SMILES: [2H]c1c([2H])c([2H])c(-c2cccc(-c3c4c([2H])c([2H])c([2H])c([2H])c4c(-c4ccccc4-n4c(-c5ccccc5)nc5ccccc54)c4c([2H])c([2H])c([2H])c([2H])c34)c2)c([2H])c1[2H]]

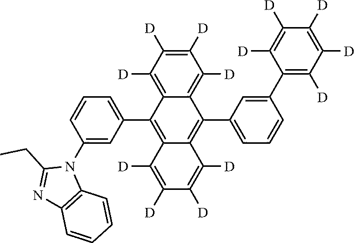 [SMILES: [2H]c1c([2H])c([2H])c(-c2cccc(-c3c4c([2H])c([2H])c([2H])c([2H])c4c(-c4cccc(-n5c(CC)nc6ccccc65)c4)c4c([2H])c([2H])c([2H])c([2H])c34)c2)c([2H])c1[2H]]

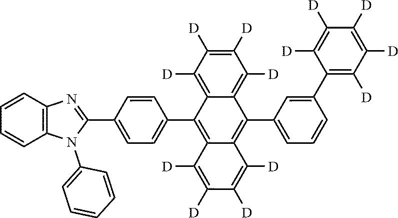 [SMILES: [2H]c1c([2H])c([2H])c(-c2cccc(-c3c4c([2H])c([2H])c([2H])c([2H])c4c(-c4ccc(-c5nc6ccccc6n5-c5ccccc5)cc4)c4c([2H])c([2H])c([2H])c([2H])c34)c2)c([2H])c1[2H]]

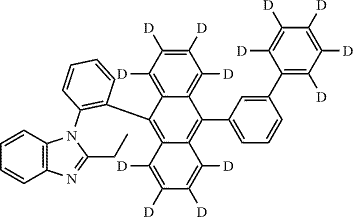 [SMILES: [2H]c1c([2H])c([2H])c(-c2cccc(-c3c4c([2H])c([2H])c([2H])c([2H])c4c(-c4ccccc4-n4c(CC)nc5ccccc54)c4c([2H])c([2H])c([2H])c([2H])c34)c2)c([2H])c1[2H]]

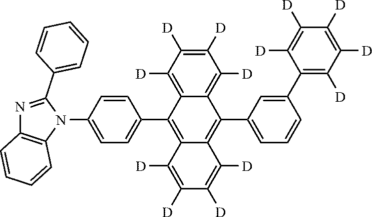 [SMILES: [2H]c1c([2H])c([2H])c(-c2cccc(-c3c4c([2H])c([2H])c([2H])c([2H])c4c(-c4ccc(-n5c(-c6ccccc6)nc6ccccc65)cc4)c4c([2H])c([2H])c([2H])c([2H])c34)c2)c([2H])c1[2H]]

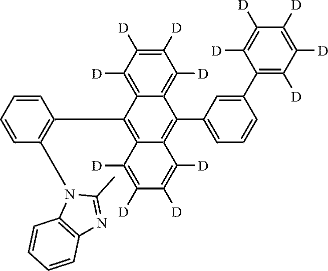 [SMILES: [2H]c1c([2H])c([2H])c(-c2cccc(-c3c4c([2H])c([2H])c([2H])c([2H])c4c(-c4ccccc4-n4c(C)nc5ccccc54)c4c([2H])c([2H])c([2H])c([2H])c34)c2)c([2H])c1[2H]]

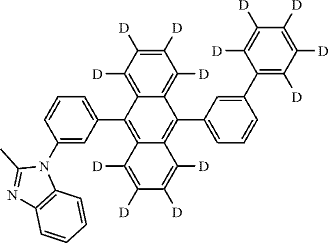 [SMILES: [2H]c1c([2H])c([2H])c(-c2cccc(-c3c4c([2H])c([2H])c([2H])c([2H])c4c(-c4cccc(-n5c(C)nc6ccccc65)c4)c4c([2H])c([2H])c([2H])c([2H])c34)c2)c([2H])c1[2H]]

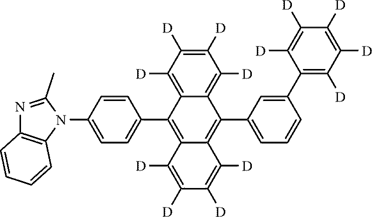 [SMILES: [2H]c1c([2H])c([2H])c(-c2cccc(-c3c4c([2H])c([2H])c([2H])c([2H])c4c(-c4ccc(-n5c(C)nc6ccccc65)cc4)c4c([2H])c([2H])c([2H])c([2H])c34)c2)c([2H])c1[2H]]

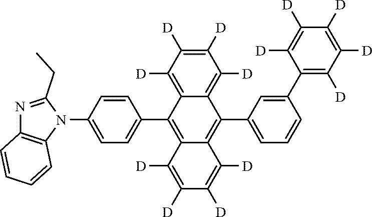 [SMILES: [2H]c1c([2H])c([2H])c(-c2cccc(-c3c4c([2H])c([2H])c([2H])c([2H])c4c(-c4ccc(-n5c(CC)nc6ccccc65)cc4)c4c([2H])c([2H])c([2H])c([2H])c34)c2)c([2H])c1[2H]]

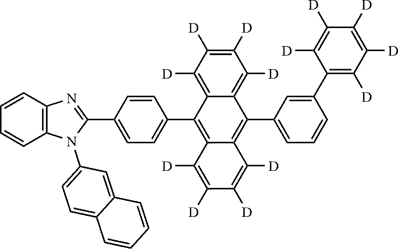 [SMILES: [2H]c1c([2H])c([2H])c(-c2cccc(-c3c4c([2H])c([2H])c([2H])c([2H])c4c(-c4ccc(-c5nc6ccccc6n5-c5ccc6ccccc6c5)cc4)c4c([2H])c([2H])c([2H])c([2H])c34)c2)c([2H])c1[2H]]

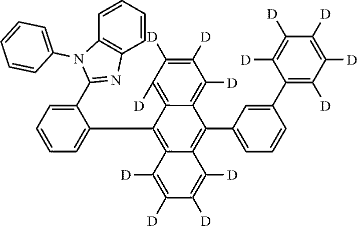 [SMILES: [2H]c1c([2H])c([2H])c(-c2cccc(-c3c4c([2H])c([2H])c([2H])c([2H])c4c(-c4ccccc4-c4nc5ccccc5n4-c4ccccc4)c4c([2H])c([2H])c([2H])c([2H])c34)c2)c([2H])c1[2H]]

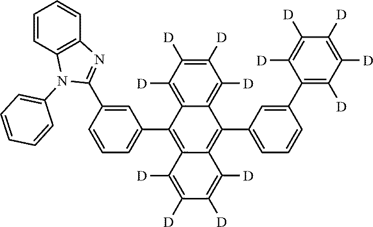 [SMILES: [2H]c1c([2H])c([2H])c(-c2cccc(-c3c4c([2H])c([2H])c([2H])c([2H])c4c(-c4cccc(-c5nc6ccccc6n5-c5ccccc5)c4)c4c([2H])c([2H])c([2H])c([2H])c34)c2)c([2H])c1[2H]]

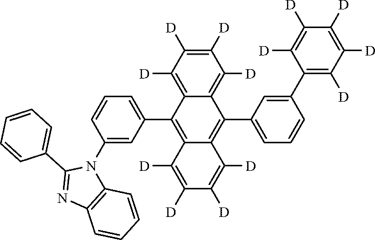 [SMILES: [2H]c1c([2H])c([2H])c(-c2cccc(-c3c4c([2H])c([2H])c([2H])c([2H])c4c(-c4cccc(-n5c(-c6ccccc6)nc6ccccc65)c4)c4c([2H])c([2H])c([2H])c([2H])c34)c2)c([2H])c1[2H]]

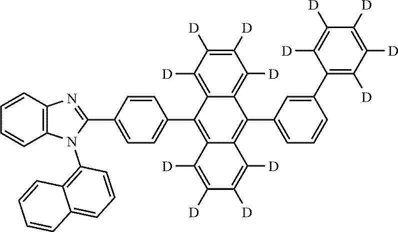 [SMILES: [2H]c1c([2H])c([2H])c(-c2cccc(-c3c4c([2H])c([2H])c([2H])c([2H])c4c(-c4ccc(-c5nc6ccccc6n5-c5cccc6ccccc56)cc4)c4c([2H])c([2H])c([2H])c([2H])c34)c2)c([2H])c1[2H]]

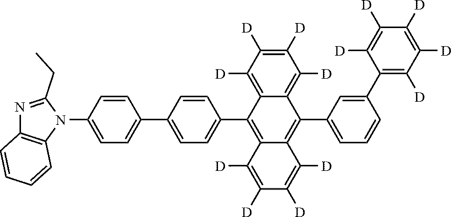 [SMILES: [2H]c1c([2H])c([2H])c(-c2cccc(-c3c4c([2H])c([2H])c([2H])c([2H])c4c(-c4ccc(-c5ccc(-n6c(CC)nc7ccccc76)cc5)cc4)c4c([2H])c([2H])c([2H])c([2H])c34)c2)c([2H])c1[2H]]

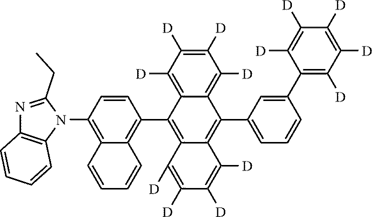 [SMILES: [2H]c1c([2H])c([2H])c(-c2cccc(-c3c4c([2H])c([2H])c([2H])c([2H])c4c(-c4ccc(-n5c(CC)nc6ccccc65)c5ccccc45)c4c([2H])c([2H])c([2H])c([2H])c34)c2)c([2H])c1[2H]]

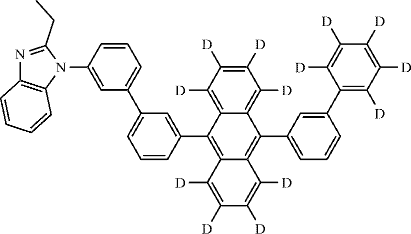 [SMILES: [2H]c1c([2H])c([2H])c(-c2cccc(-c3c4c([2H])c([2H])c([2H])c([2H])c4c(-c4cccc(-c5cccc(-n6c(CC)nc7ccccc76)c5)c4)c4c([2H])c([2H])c([2H])c([2H])c34)c2)c([2H])c1[2H]]

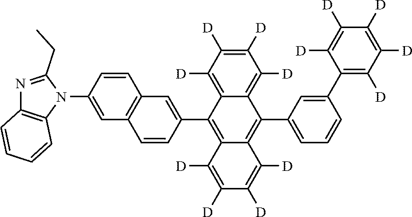 [SMILES: [2H]c1c([2H])c([2H])c(-c2cccc(-c3c4c([2H])c([2H])c([2H])c([2H])c4c(-c4ccc5cc(-n6c(CC)nc7ccccc76)ccc5c4)c4c([2H])c([2H])c([2H])c([2H])c34)c2)c([2H])c1[2H]]

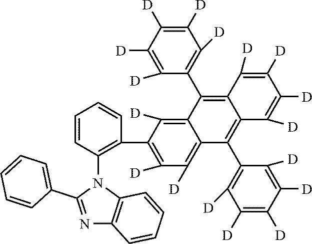 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(-c5ccccc5)nc5ccccc54)c([2H])c([2H])c23)c([2H])c1[2H]]

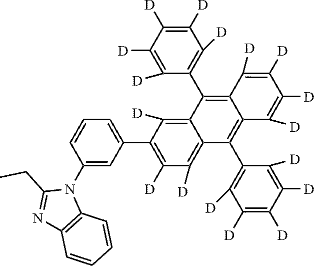 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-n5c(CC)nc6ccccc65)c4)c([2H])c([2H])c23)c([2H])c1[2H]]

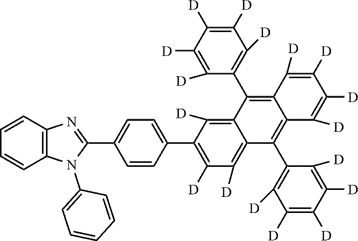 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5nc6ccccc6n5-c5ccccc5)cc4)c([2H])c([2H])c23)c([2H])c1[2H]]

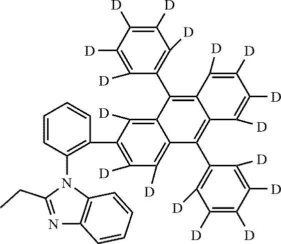 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(CC)nc5ccccc54)c([2H])c([2H])c23)c([2H])c1[2H]]

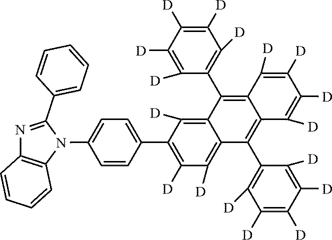 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(-c6ccccc6)nc6ccccc65)cc4)c([2H])c([2H])c23)c([2H])c1[2H]]

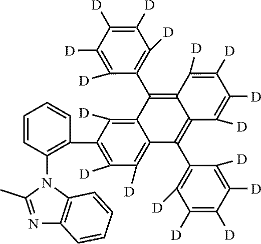 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-n4c(C)nc5ccccc54)c([2H])c([2H])c23)c([2H])c1[2H]]

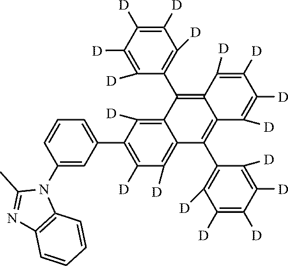 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-n5c(C)nc6ccccc65)c4)c([2H])c([2H])c23)c([2H])c1[2H]]

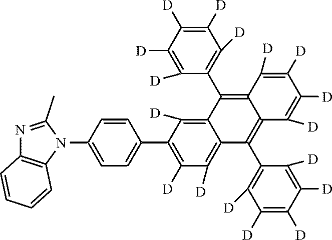 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(C)nc6ccccc65)cc4)c([2H])c([2H])c23)c([2H])c1[2H]]

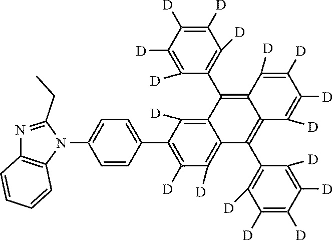 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(CC)nc6ccccc65)cc4)c([2H])c([2H])c23)c([2H])c1[2H]]

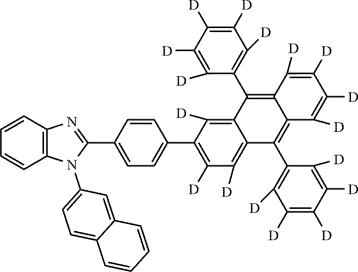 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5nc6ccccc6n5-c5ccc6ccccc6c5)cc4)c([2H])c([2H])c23)c([2H])c1[2H]]

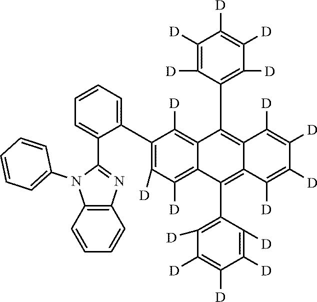 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccccc4-c4nc5ccccc5n4-c4ccccc4)c([2H])c([2H])c23)c([2H])c1[2H]]

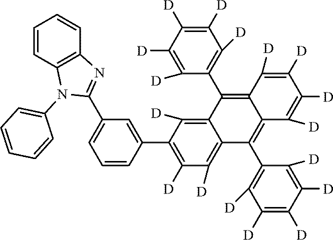 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-c5nc6ccccc6n5-c5ccccc5)c4)c([2H])c([2H])c23)c([2H])c1[2H]]

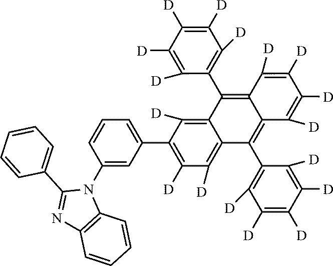 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-n5c(-c6ccccc6)nc6ccccc65)c4)c([2H])c([2H])c23)c([2H])c1[2H]]

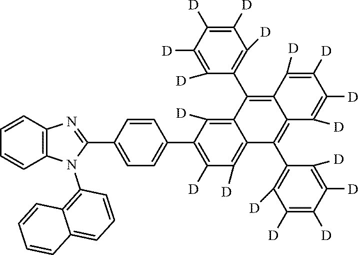 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5nc6ccccc6n5-c5cccc6ccccc56)cc4)c([2H])c([2H])c23)c([2H])c1[2H]]

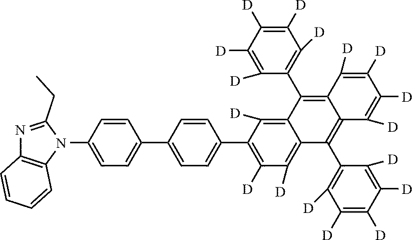 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-c5ccc(-n6c(CC)nc7ccccc76)cc5)cc4)c([2H])c([2H])c23)c([2H])c1[2H]]

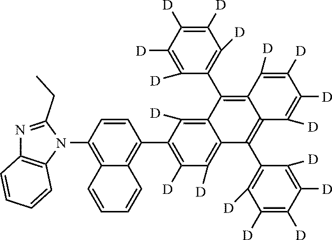 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc(-n5c(CC)nc6ccccc65)c5ccccc45)c([2H])c([2H])c23)c([2H])c1[2H]]

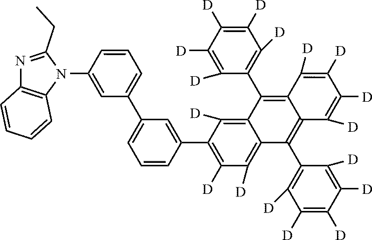 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4cccc(-c5cccc(-n6c(CC)nc7ccccc76)c5)c4)c([2H])c([2H])c23)c([2H])c1[2H]]

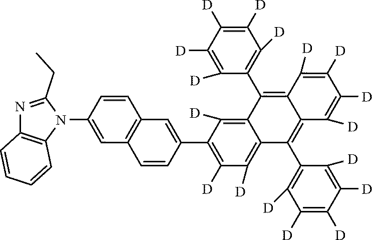 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4ccc5cc(-n6c(CC)nc7ccccc76)ccc5c4)c([2H])c([2H])c23)c([2H])c1[2H]]

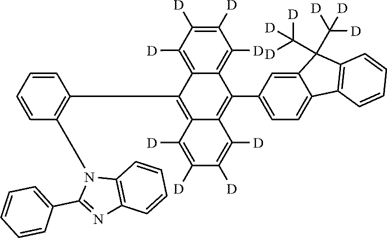 [SMILES: [2H]c1c([2H])c([2H])c2c(-c3ccccc3-n3c(-c4ccccc4)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

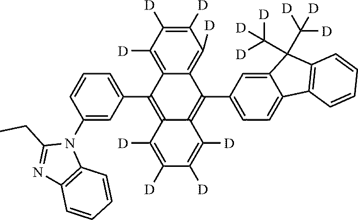 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-n4c(CC)nc5ccccc54)c3)c2c1[2H]]

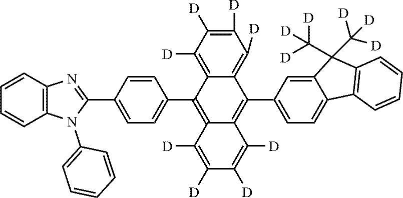 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4nc5ccccc5n4-c4ccccc4)cc3)c2c1[2H]]

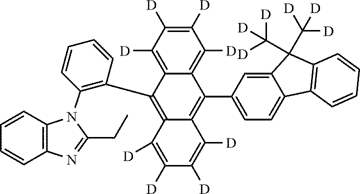 [SMILES: [2H]c1c([2H])c([2H])c2c(-c3ccccc3-n3c(CC)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

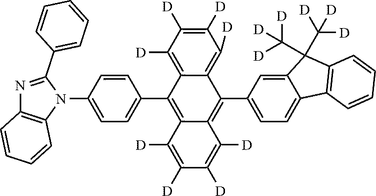 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-n4c(-c5ccccc5)nc5ccccc54)cc3)c2c1[2H]]

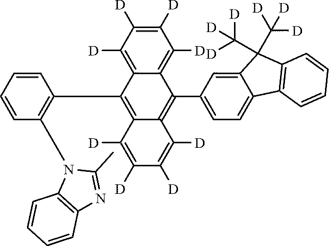 [SMILES: [2H]c1c([2H])c([2H])c2c(-c3ccccc3-n3c(C)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

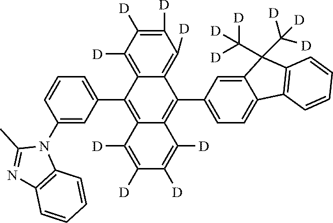 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-n4c(C)nc5ccccc54)c3)c2c1[2H]]

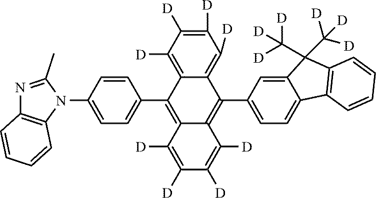 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-n4c(C)nc5ccccc54)cc3)c2c1[2H]]

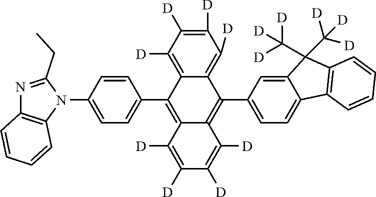 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-n4c(CC)nc5ccccc54)cc3)c2c1[2H]]

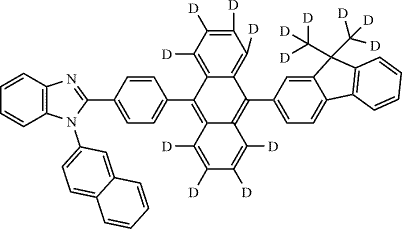 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4nc5ccccc5n4-c4ccc5ccccc5c4)cc3)c2c1[2H]]

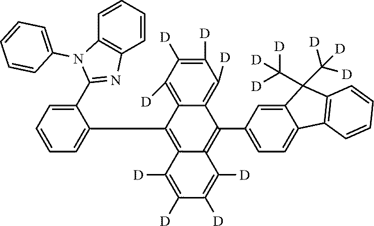 [SMILES: [2H]c1c([2H])c([2H])c2c(-c3ccccc3-c3nc4ccccc4n3-c3ccccc3)c3c([2H])c([2H])c([2H])c([2H])c3c(-c3ccc4c(c3)C(C([2H])([2H])[2H])(C([2H])([2H])[2H])c3ccccc3-4)c2c1[2H]]

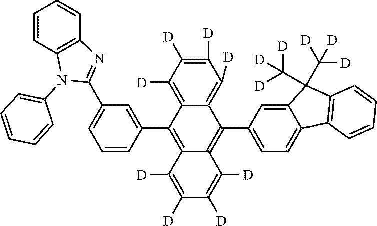 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-c4nc5ccccc5n4-c4ccccc4)c3)c2c1[2H]]

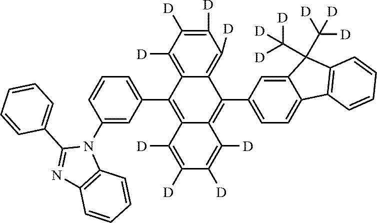 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-n4c(-c5ccccc5)nc5ccccc54)c3)c2c1[2H]]

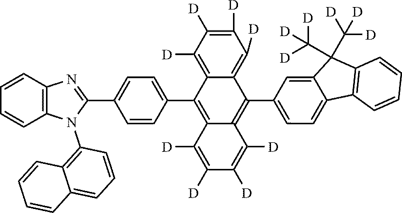 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4nc5ccccc5n4-c4cccc5ccccc45)cc3)c2c1[2H]]

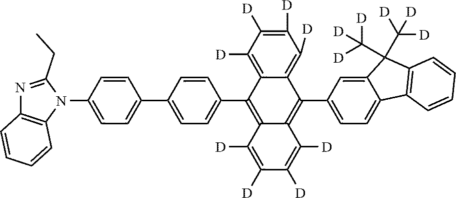 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3ccc(-c4ccc(-n5c(CC)nc6ccccc65)cc4)cc3)c2c1[2H]]

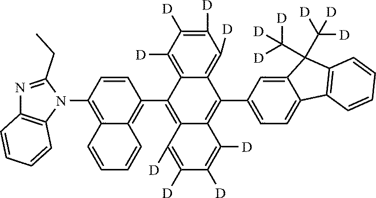 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccc4c(c3)C(C([2H])([2H])[2H])(C([2H])([2H])[2H])c3ccccc3-4)c2c1[2H]]

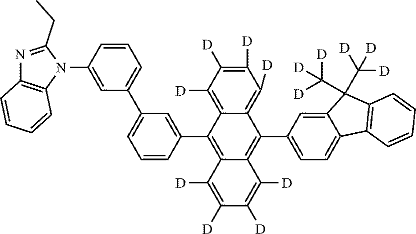 [SMILES: [2H]c1c([2H])c([2H])c2c(-c3ccc4c(c3)C(C([2H])([2H])[2H])(C([2H])([2H])[2H])c3ccccc3-4)c3c([2H])c([2H])c([2H])c([2H])c3c(-c3cccc(-c4cccc(-n5c(CC)nc6ccccc65)c4)c3)c2c1[2H]]

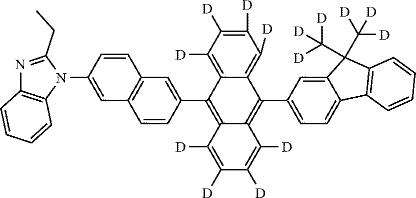 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3ccc4c(c3)C(C([2H])([2H])[2H])(C([2H])([2H])[2H])c3ccccc3-4)c2c1[2H]]

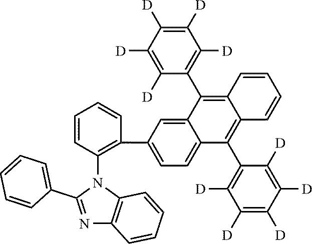 [SMILES: [2H]c1c([2H])c([2H])c(-c2c3ccccc3c(-c3c([2H])c([2H])c([2H])c([2H])c3[2H])c3cc(-c4ccccc4-n4c(-c5ccccc5)nc5ccccc54)ccc23)c([2H])c1[2H]]

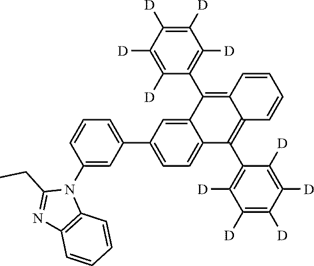 [SMILES: [2H]c1c([2H])c([2H])c(-c2c3ccccc3c(-c3c([2H])c([2H])c([2H])c([2H])c3[2H])c3cc(-c4cccc(-n5c(CC)nc6ccccc65)c4)ccc23)c([2H])c1[2H]]

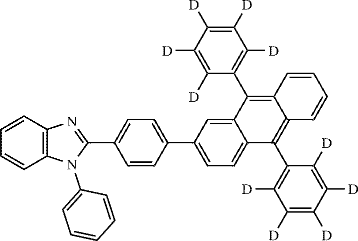 [SMILES: [2H]c1c([2H])c([2H])c(-c2c3ccccc3c(-c3c([2H])c([2H])c([2H])c([2H])c3[2H])c3cc(-c4ccc(-c5nc6ccccc6n5-c5ccccc5)cc4)ccc23)c([2H])c1[2H]]

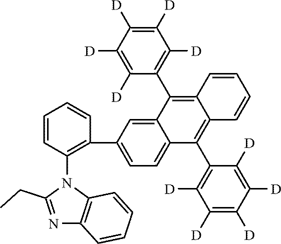 [SMILES: [2H]c1c([2H])c([2H])c(-c2c3ccccc3c(-c3c([2H])c([2H])c([2H])c([2H])c3[2H])c3cc(-c4ccccc4-n4c(CC)nc5ccccc54)ccc23)c([2H])c1[2H]]

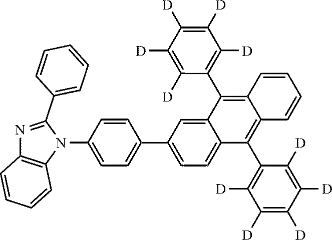 [SMILES: [2H]c1c([2H])c([2H])c(-c2c3ccccc3c(-c3c([2H])c([2H])c([2H])c([2H])c3[2H])c3cc(-c4ccc(-n5c(-c6ccccc6)nc6ccccc65)cc4)ccc23)c([2H])c1[2H]]

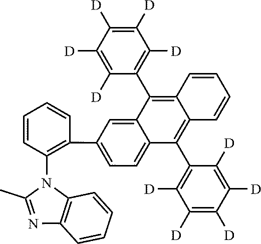 [SMILES: [2H]c1c([2H])c([2H])c(-c2c3ccccc3c(-c3c([2H])c([2H])c([2H])c([2H])c3[2H])c3cc(-c4ccccc4-n4c(C)nc5ccccc54)ccc23)c([2H])c1[2H]]

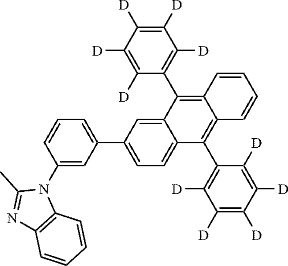 [SMILES: [2H]c1c([2H])c([2H])c(-c2c3ccccc3c(-c3c([2H])c([2H])c([2H])c([2H])c3[2H])c3cc(-c4cccc(-n5c(C)nc6ccccc65)c4)ccc23)c([2H])c1[2H]]

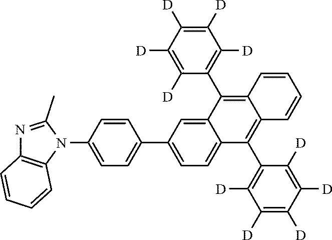 [SMILES: [2H]c1c([2H])c([2H])c(-c2c3ccccc3c(-c3c([2H])c([2H])c([2H])c([2H])c3[2H])c3cc(-c4ccc(-n5c(C)nc6ccccc65)cc4)ccc23)c([2H])c1[2H]]

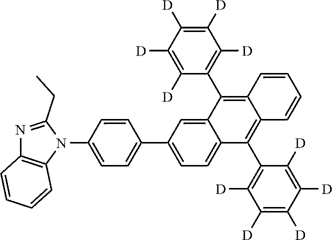 [SMILES: [2H]c1c([2H])c([2H])c(-c2c3ccccc3c(-c3c([2H])c([2H])c([2H])c([2H])c3[2H])c3cc(-c4ccc(-n5c(CC)nc6ccccc65)cc4)ccc23)c([2H])c1[2H]]

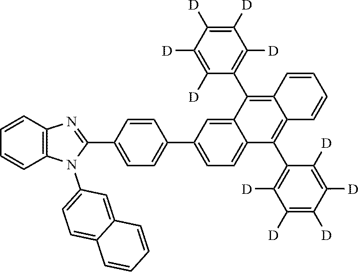 [SMILES: [2H]c1c([2H])c([2H])c(-c2c3ccccc3c(-c3c([2H])c([2H])c([2H])c([2H])c3[2H])c3cc(-c4ccc(-c5nc6ccccc6n5-c5ccc6ccccc6c5)cc4)ccc23)c([2H])c1[2H]]

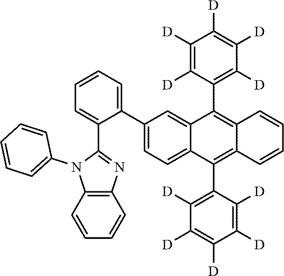 [SMILES: [2H]c1c([2H])c([2H])c(-c2c3ccccc3c(-c3c([2H])c([2H])c([2H])c([2H])c3[2H])c3cc(-c4ccccc4-c4nc5ccccc5n4-c4ccccc4)ccc23)c([2H])c1[2H]]

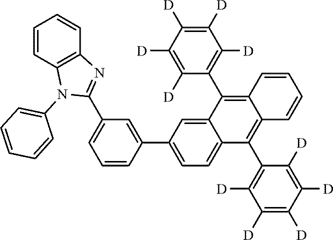 [SMILES: [2H]c1c([2H])c([2H])c(-c2c3ccccc3c(-c3c([2H])c([2H])c([2H])c([2H])c3[2H])c3cc(-c4cccc(-c5nc6ccccc6n5-c5ccccc5)c4)ccc23)c([2H])c1[2H]]

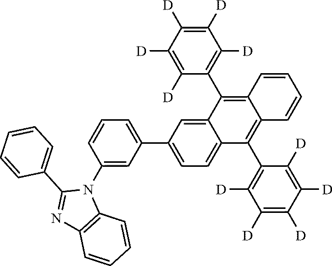 [SMILES: [2H]c1c([2H])c([2H])c(-c2c3ccccc3c(-c3c([2H])c([2H])c([2H])c([2H])c3[2H])c3cc(-c4cccc(-n5c(-c6ccccc6)nc6ccccc65)c4)ccc23)c([2H])c1[2H]]

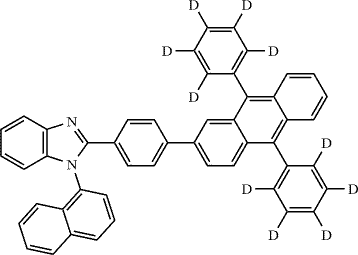 [SMILES: [2H]c1c([2H])c([2H])c(-c2c3ccccc3c(-c3c([2H])c([2H])c([2H])c([2H])c3[2H])c3cc(-c4ccc(-c5nc6ccccc6n5-c5cccc6ccccc56)cc4)ccc23)c([2H])c1[2H]]

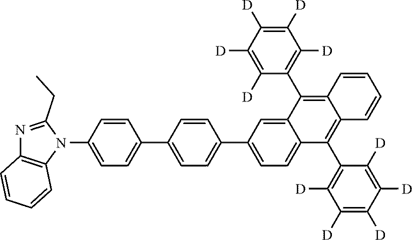 [SMILES: [2H]c1c([2H])c([2H])c(-c2c3ccccc3c(-c3c([2H])c([2H])c([2H])c([2H])c3[2H])c3cc(-c4ccc(-c5ccc(-n6c(CC)nc7ccccc76)cc5)cc4)ccc23)c([2H])c1[2H]]

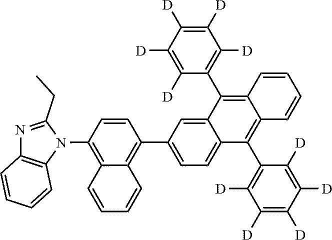 [SMILES: [2H]c1c([2H])c([2H])c(-c2c3ccccc3c(-c3c([2H])c([2H])c([2H])c([2H])c3[2H])c3cc(-c4ccc(-n5c(CC)nc6ccccc65)c5ccccc45)ccc23)c([2H])c1[2H]]

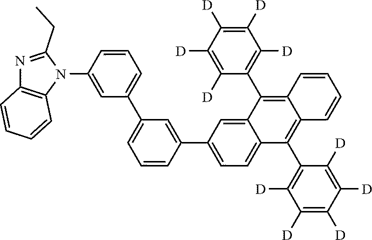 [SMILES: [2H]c1c([2H])c([2H])c(-c2c3ccccc3c(-c3c([2H])c([2H])c([2H])c([2H])c3[2H])c3cc(-c4cccc(-c5cccc(-n6c(CC)nc7ccccc76)c5)c4)ccc23)c([2H])c1[2H]]

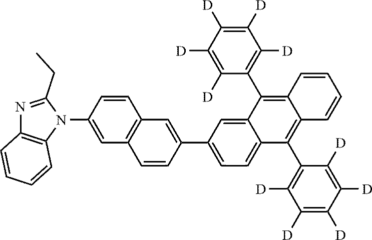 [SMILES: [2H]c1c([2H])c([2H])c(-c2c3ccccc3c(-c3c([2H])c([2H])c([2H])c([2H])c3[2H])c3cc(-c4ccc5cc(-n6c(CC)nc7ccccc76)ccc5c4)ccc23)c([2H])c1[2H]]

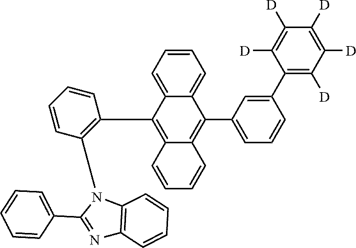 [SMILES: [2H]c1c([2H])c([2H])c(-c2cccc(-c3c4ccccc4c(-c4ccccc4-n4c(-c5ccccc5)nc5ccccc54)c4ccccc34)c2)c([2H])c1[2H]]

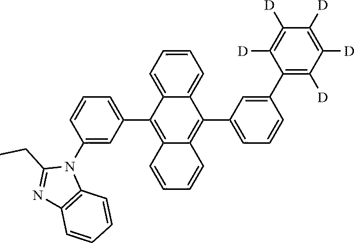 [SMILES: [2H]c1c([2H])c([2H])c(-c2cccc(-c3c4ccccc4c(-c4cccc(-n5c(CC)nc6ccccc65)c4)c4ccccc34)c2)c([2H])c1[2H]]

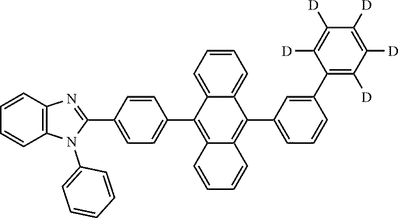 [SMILES: [2H]c1c([2H])c([2H])c(-c2cccc(-c3c4ccccc4c(-c4ccc(-c5nc6ccccc6n5-c5ccccc5)cc4)c4ccccc34)c2)c([2H])c1[2H]]

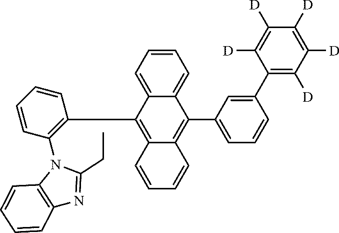 [SMILES: [2H]c1c([2H])c([2H])c(-c2cccc(-c3c4ccccc4c(-c4ccccc4-n4c(CC)nc5ccccc54)c4ccccc34)c2)c([2H])c1[2H]]

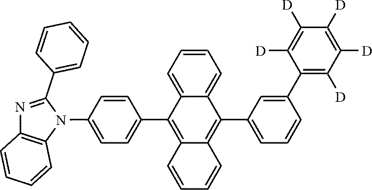 [SMILES: [2H]c1c([2H])c([2H])c(-c2cccc(-c3c4ccccc4c(-c4ccc(-n5c(-c6ccccc6)nc6ccccc65)cc4)c4ccccc34)c2)c([2H])c1[2H]]

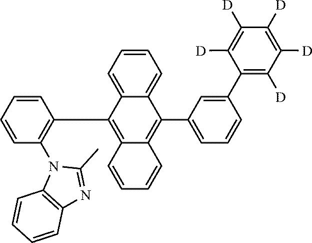 [SMILES: [2H]c1c([2H])c([2H])c(-c2cccc(-c3c4ccccc4c(-c4ccccc4-n4c(C)nc5ccccc54)c4ccccc34)c2)c([2H])c1[2H]]

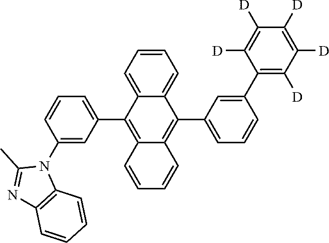 [SMILES: [2H]c1c([2H])c([2H])c(-c2cccc(-c3c4ccccc4c(-c4cccc(-n5c(C)nc6ccccc65)c4)c4ccccc34)c2)c([2H])c1[2H]]

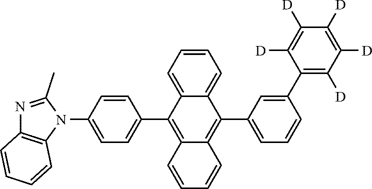 [SMILES: [2H]c1c([2H])c([2H])c(-c2cccc(-c3c4ccccc4c(-c4ccc(-n5c(C)nc6ccccc65)cc4)c4ccccc34)c2)c([2H])c1[2H]]

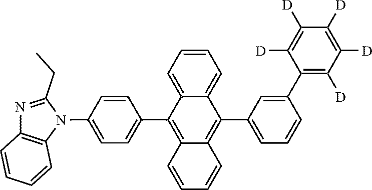 [SMILES: [2H]c1c([2H])c([2H])c(-c2cccc(-c3c4ccccc4c(-c4ccc(-n5c(CC)nc6ccccc65)cc4)c4ccccc34)c2)c([2H])c1[2H]]

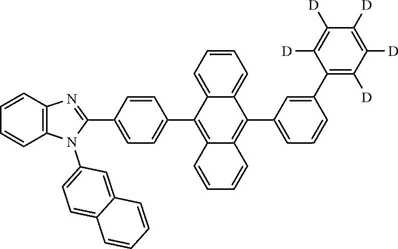 [SMILES: [2H]c1c([2H])c([2H])c(-c2cccc(-c3c4ccccc4c(-c4ccc(-c5nc6ccccc6n5-c5ccc6ccccc6c5)cc4)c4ccccc34)c2)c([2H])c1[2H]]

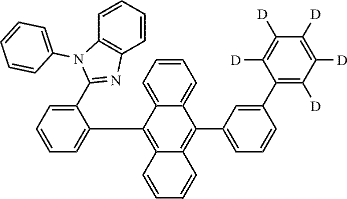 [SMILES: [2H]c1c([2H])c([2H])c(-c2cccc(-c3c4ccccc4c(-c4ccccc4-c4nc5ccccc5n4-c4ccccc4)c4ccccc34)c2)c([2H])c1[2H]]

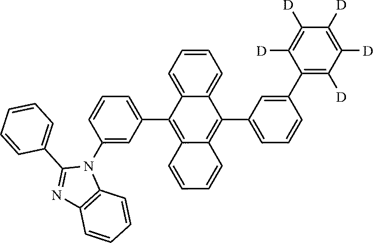 [SMILES: [2H]c1c([2H])c([2H])c(-c2cccc(-c3c4ccccc4c(-c4cccc(-n5c(-c6ccccc6)nc6ccccc65)c4)c4ccccc34)c2)c([2H])c1[2H]]

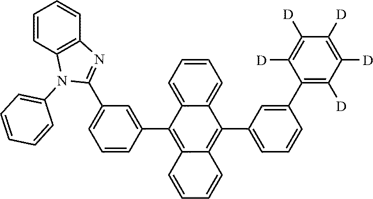 [SMILES: [2H]c1c([2H])c([2H])c(-c2cccc(-c3c4ccccc4c(-c4cccc(-c5nc6ccccc6n5-c5ccccc5)c4)c4ccccc34)c2)c([2H])c1[2H]]

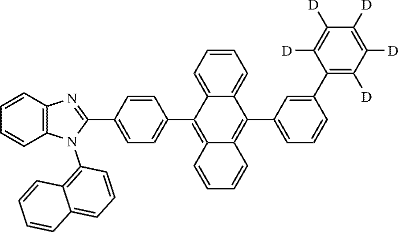 [SMILES: [2H]c1c([2H])c([2H])c(-c2cccc(-c3c4ccccc4c(-c4ccc(-c5nc6ccccc6n5-c5cccc6ccccc56)cc4)c4ccccc34)c2)c([2H])c1[2H]]

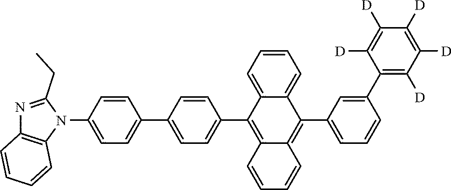 [SMILES: [2H]c1c([2H])c([2H])c(-c2cccc(-c3c4ccccc4c(-c4ccc(-c5ccc(-n6c(CC)nc7ccccc76)cc5)cc4)c4ccccc34)c2)c([2H])c1[2H]]

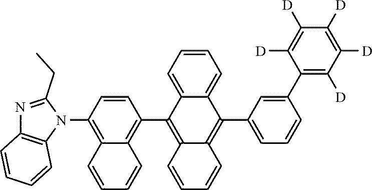 [SMILES: [2H]c1c([2H])c([2H])c(-c2cccc(-c3c4ccccc4c(-c4ccc(-n5c(CC)nc6ccccc65)c5ccccc45)c4ccccc34)c2)c([2H])c1[2H]]

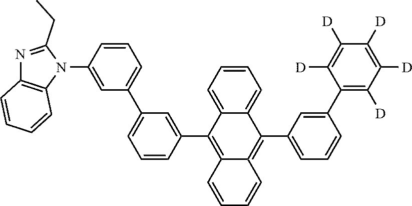 [SMILES: [2H]c1c([2H])c([2H])c(-c2cccc(-c3c4ccccc4c(-c4cccc(-c5cccc(-n6c(CC)nc7ccccc76)c5)c4)c4ccccc34)c2)c([2H])c1[2H]]

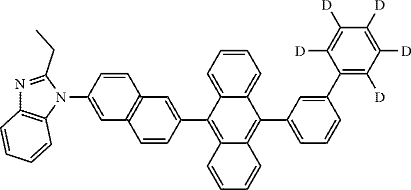 [SMILES: [2H]c1c([2H])c([2H])c(-c2cccc(-c3c4ccccc4c(-c4ccc5cc(-n6c(CC)nc7ccccc76)ccc5c4)c4ccccc34)c2)c([2H])c1[2H]]

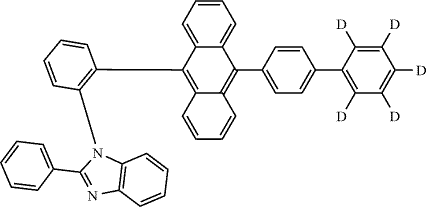 [SMILES: [2H]c1c([2H])c([2H])c(-c2ccc(-c3c4ccccc4c(-c4ccccc4-n4c(-c5ccccc5)nc5ccccc54)c4ccccc34)cc2)c([2H])c1[2H]]

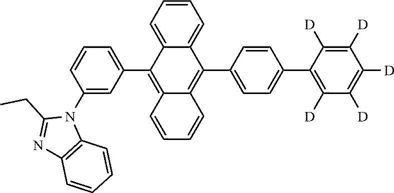 [SMILES: [2H]c1c([2H])c([2H])c(-c2ccc(-c3c4ccccc4c(-c4cccc(-n5c(CC)nc6ccccc65)c4)c4ccccc34)cc2)c([2H])c1[2H]]

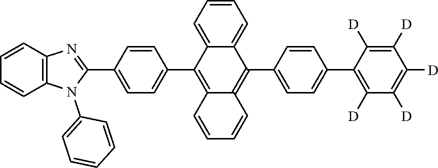 [SMILES: [2H]c1c([2H])c([2H])c(-c2ccc(-c3c4ccccc4c(-c4ccc(-c5nc6ccccc6n5-c5ccccc5)cc4)c4ccccc34)cc2)c([2H])c1[2H]]

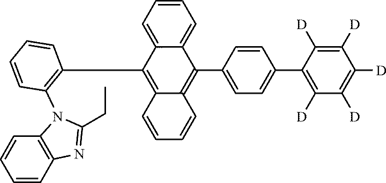 [SMILES: [2H]c1c([2H])c([2H])c(-c2ccc(-c3c4ccccc4c(-c4ccccc4-n4c(CC)nc5ccccc54)c4ccccc34)cc2)c([2H])c1[2H]]

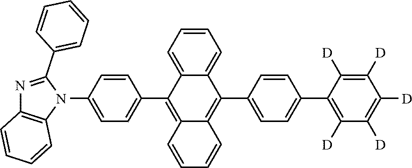 [SMILES: [2H]c1c([2H])c([2H])c(-c2ccc(-c3c4ccccc4c(-c4ccc(-n5c(-c6ccccc6)nc6ccccc65)cc4)c4ccccc34)cc2)c([2H])c1[2H]]

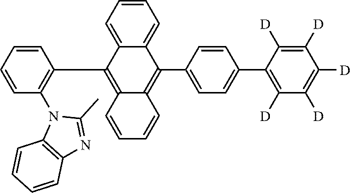 [SMILES: [2H]c1c([2H])c([2H])c(-c2ccc(-c3c4ccccc4c(-c4ccccc4-n4c(C)nc5ccccc54)c4ccccc34)cc2)c([2H])c1[2H]]

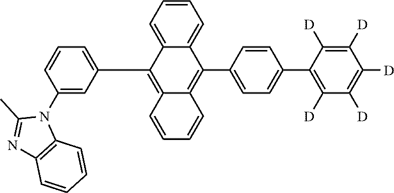 [SMILES: [2H]c1c([2H])c([2H])c(-c2ccc(-c3c4ccccc4c(-c4cccc(-n5c(C)nc6ccccc65)c4)c4ccccc34)cc2)c([2H])c1[2H]]

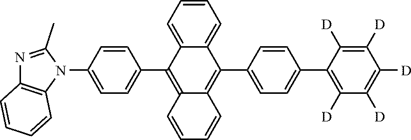 [SMILES: [2H]c1c([2H])c([2H])c(-c2ccc(-c3c4ccccc4c(-c4ccc(-n5c(C)nc6ccccc65)cc4)c4ccccc34)cc2)c([2H])c1[2H]]

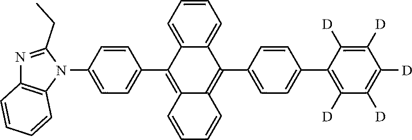 [SMILES: [2H]c1c([2H])c([2H])c(-c2ccc(-c3c4ccccc4c(-c4ccc(-n5c(CC)nc6ccccc65)cc4)c4ccccc34)cc2)c([2H])c1[2H]]

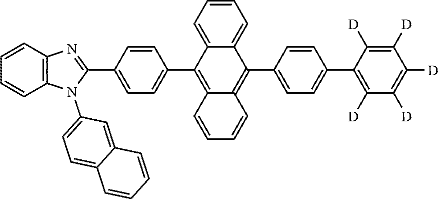 [SMILES: [2H]c1c([2H])c([2H])c(-c2ccc(-c3c4ccccc4c(-c4ccc(-c5nc6ccccc6n5-c5ccc6ccccc6c5)cc4)c4ccccc34)cc2)c([2H])c1[2H]]

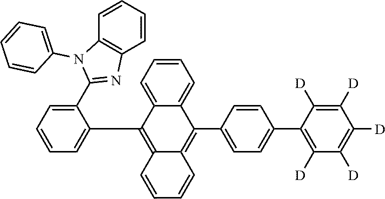 [SMILES: [2H]c1c([2H])c([2H])c(-c2ccc(-c3c4ccccc4c(-c4ccccc4-c4nc5ccccc5n4-c4ccccc4)c4ccccc34)cc2)c([2H])c1[2H]]

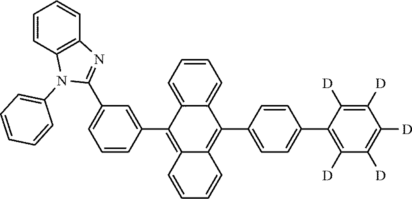 [SMILES: [2H]c1c([2H])c([2H])c(-c2ccc(-c3c4ccccc4c(-c4cccc(-c5nc6ccccc6n5-c5ccccc5)c4)c4ccccc34)cc2)c([2H])c1[2H]]

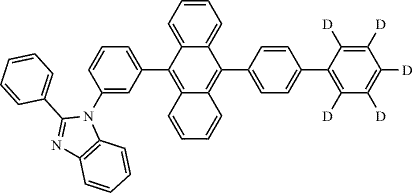 [SMILES: [2H]c1c([2H])c([2H])c(-c2ccc(-c3c4ccccc4c(-c4cccc(-n5c(-c6ccccc6)nc6ccccc65)c4)c4ccccc34)cc2)c([2H])c1[2H]]

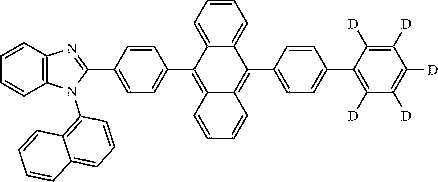 [SMILES: [2H]c1c([2H])c([2H])c(-c2ccc(-c3c4ccccc4c(-c4ccc(-c5nc6ccccc6n5-c5cccc6ccccc56)cc4)c4ccccc34)cc2)c([2H])c1[2H]]

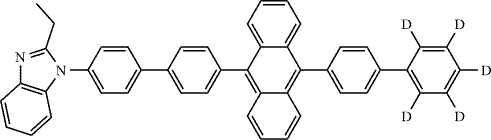 [SMILES: [2H]c1c([2H])c([2H])c(-c2ccc(-c3c4ccccc4c(-c4ccc(-c5ccc(-n6c(CC)nc7ccccc76)cc5)cc4)c4ccccc34)cc2)c([2H])c1[2H]]

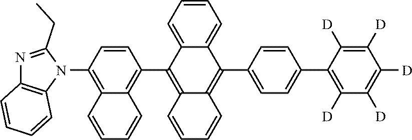 [SMILES: [2H]c1c([2H])c([2H])c(-c2ccc(-c3c4ccccc4c(-c4ccc(-n5c(CC)nc6ccccc65)c5ccccc45)c4ccccc34)cc2)c([2H])c1[2H]]

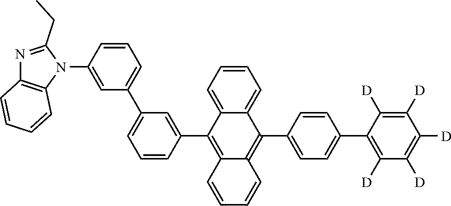 [SMILES: [2H]c1c([2H])c([2H])c(-c2ccc(-c3c4ccccc4c(-c4cccc(-c5cccc(-n6c(CC)nc7ccccc76)c5)c4)c4ccccc34)cc2)c([2H])c1[2H]]

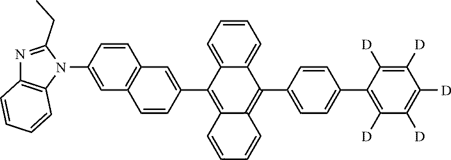 [SMILES: [2H]c1c([2H])c([2H])c(-c2ccc(-c3c4ccccc4c(-c4ccc5cc(-n6c(CC)nc7ccccc76)ccc5c4)c4ccccc34)cc2)c([2H])c1[2H]]

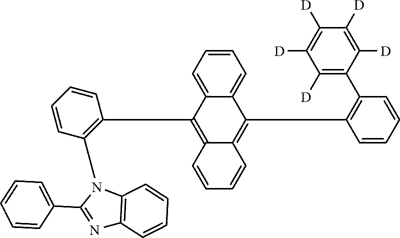 [SMILES: [2H]c1c([2H])c([2H])c(-c2ccccc2-c2c3ccccc3c(-c3ccccc3-n3c(-c4ccccc4)nc4ccccc43)c3ccccc23)c([2H])c1[2H]]

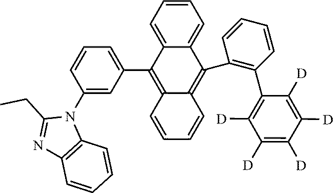 [SMILES: [2H]c1c([2H])c([2H])c(-c2ccccc2-c2c3ccccc3c(-c3cccc(-n4c(CC)nc5ccccc54)c3)c3ccccc23)c([2H])c1[2H]]

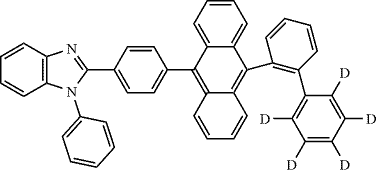 [SMILES: [2H]c1c([2H])c([2H])c(-c2ccccc2-c2c3ccccc3c(-c3ccc(-c4nc5ccccc5n4-c4ccccc4)cc3)c3ccccc23)c([2H])c1[2H]]

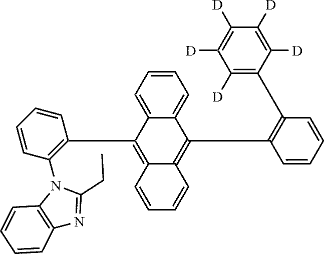 [SMILES: [2H]c1c([2H])c([2H])c(-c2ccccc2-c2c3ccccc3c(-c3ccccc3-n3c(CC)nc4ccccc43)c3ccccc23)c([2H])c1[2H]]

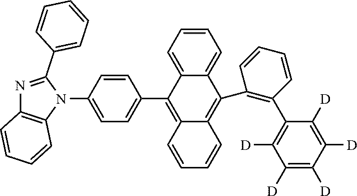 [SMILES: [2H]c1c([2H])c([2H])c(-c2ccccc2-c2c3ccccc3c(-c3ccc(-n4c(-c5ccccc5)nc5ccccc54)cc3)c3ccccc23)c([2H])c1[2H]]

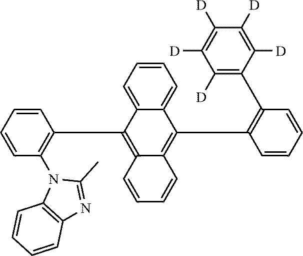 [SMILES: [2H]c1c([2H])c([2H])c(-c2ccccc2-c2c3ccccc3c(-c3ccccc3-n3c(C)nc4ccccc43)c3ccccc23)c([2H])c1[2H]]

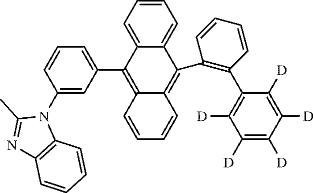 [SMILES: [2H]c1c([2H])c([2H])c(-c2ccccc2-c2c3ccccc3c(-c3cccc(-n4c(C)nc5ccccc54)c3)c3ccccc23)c([2H])c1[2H]]

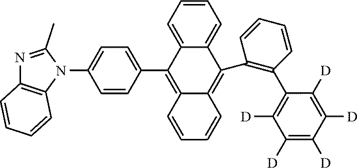 [SMILES: [2H]c1c([2H])c([2H])c(-c2ccccc2-c2c3ccccc3c(-c3ccc(-n4c(C)nc5ccccc54)cc3)c3ccccc23)c([2H])c1[2H]]

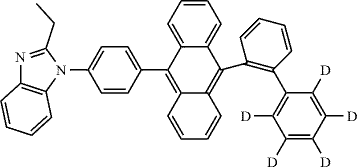 [SMILES: [2H]c1c([2H])c([2H])c(-c2ccccc2-c2c3ccccc3c(-c3ccc(-n4c(CC)nc5ccccc54)cc3)c3ccccc23)c([2H])c1[2H]]

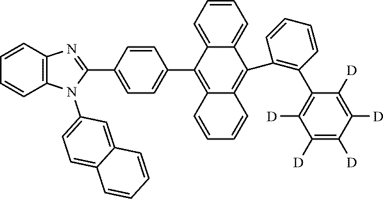 [SMILES: [2H]c1c([2H])c([2H])c(-c2ccccc2-c2c3ccccc3c(-c3ccc(-c4nc5ccccc5n4-c4ccc5ccccc5c4)cc3)c3ccccc23)c([2H])c1[2H]]

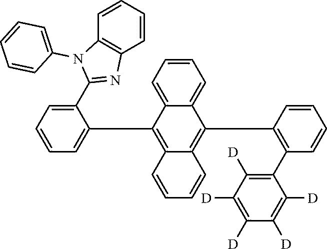 [SMILES: [2H]c1c([2H])c([2H])c(-c2ccccc2-c2c3ccccc3c(-c3ccccc3-c3nc4ccccc4n3-c3ccccc3)c3ccccc23)c([2H])c1[2H]]

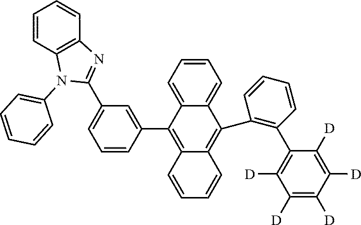 [SMILES: [2H]c1c([2H])c([2H])c(-c2ccccc2-c2c3ccccc3c(-c3cccc(-c4nc5ccccc5n4-c4ccccc4)c3)c3ccccc23)c([2H])c1[2H]]

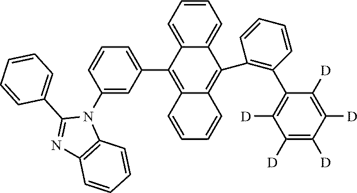 [SMILES: [2H]c1c([2H])c([2H])c(-c2ccccc2-c2c3ccccc3c(-c3cccc(-n4c(-c5ccccc5)nc5ccccc54)c3)c3ccccc23)c([2H])c1[2H]]

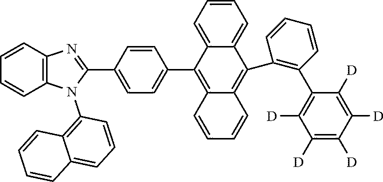 [SMILES: [2H]c1c([2H])c([2H])c(-c2ccccc2-c2c3ccccc3c(-c3ccc(-c4nc5ccccc5n4-c4cccc5ccccc45)cc3)c3ccccc23)c([2H])c1[2H]]

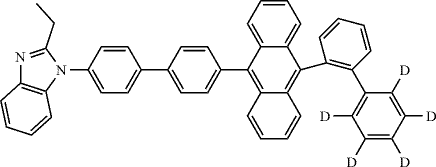 [SMILES: [2H]c1c([2H])c([2H])c(-c2ccccc2-c2c3ccccc3c(-c3ccc(-c4ccc(-n5c(CC)nc6ccccc65)cc4)cc3)c3ccccc23)c([2H])c1[2H]]

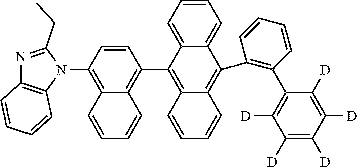 [SMILES: [2H]c1c([2H])c([2H])c(-c2ccccc2-c2c3ccccc3c(-c3ccc(-n4c(CC)nc5ccccc54)c4ccccc34)c3ccccc23)c([2H])c1[2H]]

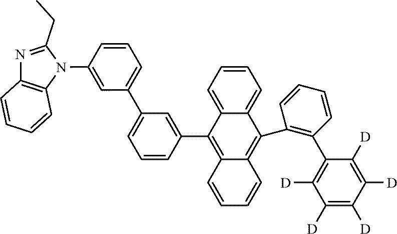 [SMILES: [2H]c1c([2H])c([2H])c(-c2ccccc2-c2c3ccccc3c(-c3cccc(-c4cccc(-n5c(CC)nc6ccccc65)c4)c3)c3ccccc23)c([2H])c1[2H]]

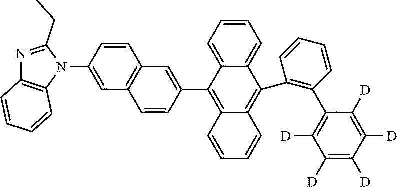 [SMILES: [2H]c1c([2H])c([2H])c(-c2ccccc2-c2c3ccccc3c(-c3ccc4cc(-n5c(CC)nc6ccccc65)ccc4c3)c3ccccc23)c([2H])c1[2H]]

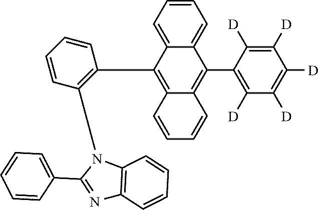 [SMILES: [2H]c1c([2H])c([2H])c(-c2c3ccccc3c(-c3ccccc3-n3c(-c4ccccc4)nc4ccccc43)c3ccccc23)c([2H])c1[2H]]

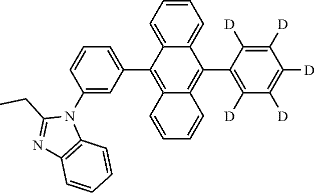 [SMILES: [2H]c1c([2H])c([2H])c(-c2c3ccccc3c(-c3cccc(-n4c(CC)nc5ccccc54)c3)c3ccccc23)c([2H])c1[2H]]

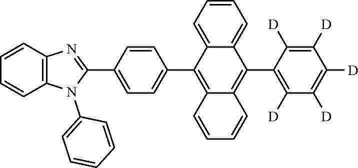 [SMILES: [2H]c1c([2H])c([2H])c(-c2c3ccccc3c(-c3ccc(-c4nc5ccccc5n4-c4ccccc4)cc3)c3ccccc23)c([2H])c1[2H]]

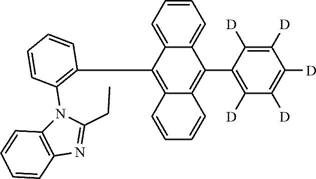 [SMILES: [2H]c1c([2H])c([2H])c(-c2c3ccccc3c(-c3ccccc3-n3c(CC)nc4ccccc43)c3ccccc23)c([2H])c1[2H]]

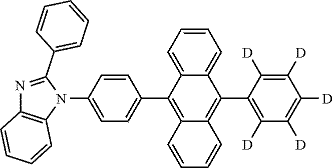 [SMILES: [2H]c1c([2H])c([2H])c(-c2c3ccccc3c(-c3ccc(-n4c(-c5ccccc5)nc5ccccc54)cc3)c3ccccc23)c([2H])c1[2H]]

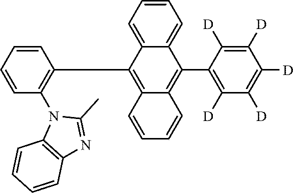 [SMILES: [2H]c1c([2H])c([2H])c(-c2c3ccccc3c(-c3ccccc3-n3c(C)nc4ccccc43)c3ccccc23)c([2H])c1[2H]]

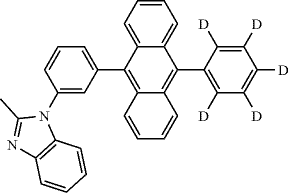 [SMILES: [2H]c1c([2H])c([2H])c(-c2c3ccccc3c(-c3cccc(-n4c(C)nc5ccccc54)c3)c3ccccc23)c([2H])c1[2H]]

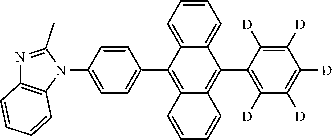 [SMILES: [2H]c1c([2H])c([2H])c(-c2c3ccccc3c(-c3ccc(-n4c(C)nc5ccccc54)cc3)c3ccccc23)c([2H])c1[2H]]

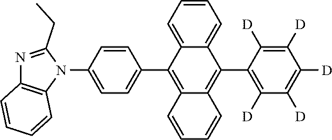 [SMILES: [2H]c1c([2H])c([2H])c(-c2c3ccccc3c(-c3ccc(-n4c(CC)nc5ccccc54)cc3)c3ccccc23)c([2H])c1[2H]]

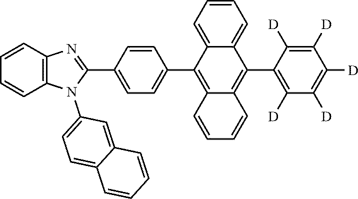 [SMILES: [2H]c1c([2H])c([2H])c(-c2c3ccccc3c(-c3ccc(-c4nc5ccccc5n4-c4ccc5ccccc5c4)cc3)c3ccccc23)c([2H])c1[2H]]

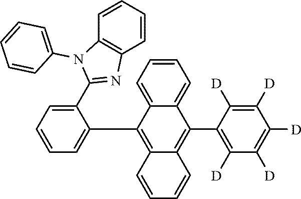 [SMILES: [2H]c1c([2H])c([2H])c(-c2c3ccccc3c(-c3ccccc3-c3nc4ccccc4n3-c3ccccc3)c3ccccc23)c([2H])c1[2H]]

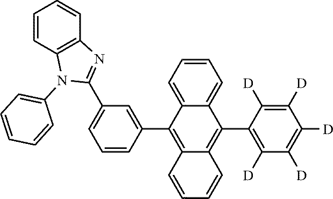 [SMILES: [2H]c1c([2H])c([2H])c(-c2c3ccccc3c(-c3cccc(-c4nc5ccccc5n4-c4ccccc4)c3)c3ccccc23)c([2H])c1[2H]]

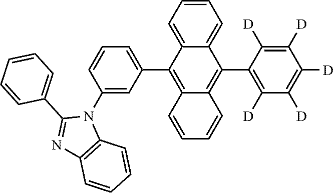 [SMILES: [2H]c1c([2H])c([2H])c(-c2c3ccccc3c(-c3cccc(-n4c(-c5ccccc5)nc5ccccc54)c3)c3ccccc23)c([2H])c1[2H]]

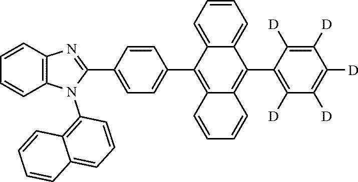 [SMILES: [2H]c1c([2H])c([2H])c(-c2c3ccccc3c(-c3ccc(-c4nc5ccccc5n4-c4cccc5ccccc45)cc3)c3ccccc23)c([2H])c1[2H]]

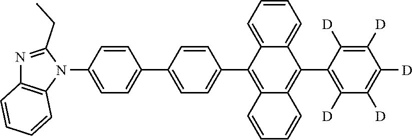 [SMILES: [2H]c1c([2H])c([2H])c(-c2c3ccccc3c(-c3ccc(-c4ccc(-n5c(CC)nc6ccccc65)cc4)cc3)c3ccccc23)c([2H])c1[2H]]

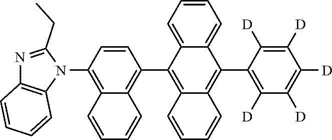 [SMILES: [2H]c1c([2H])c([2H])c(-c2c3ccccc3c(-c3ccc(-n4c(CC)nc5ccccc54)c4ccccc34)c3ccccc23)c([2H])c1[2H]]

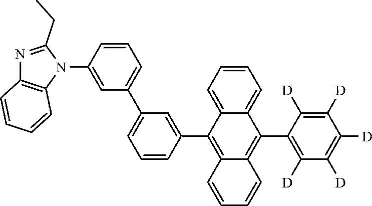 [SMILES: [2H]c1c([2H])c([2H])c(-c2c3ccccc3c(-c3cccc(-c4cccc(-n5c(CC)nc6ccccc65)c4)c3)c3ccccc23)c([2H])c1[2H]]

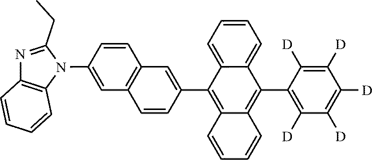 [SMILES: [2H]c1c([2H])c([2H])c(-c2c3ccccc3c(-c3ccc4cc(-n5c(CC)nc6ccccc65)ccc4c3)c3ccccc23)c([2H])c1[2H]]

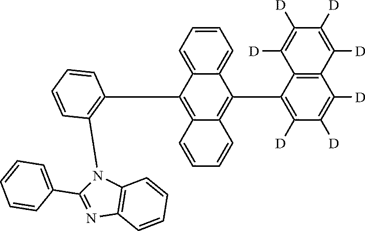 [SMILES: [2H]c1c([2H])c([2H])c2c(-c3c4ccccc4c(-c4ccccc4-n4c(-c5ccccc5)nc5ccccc54)c4ccccc34)c([2H])c([2H])c([2H])c2c1[2H]]

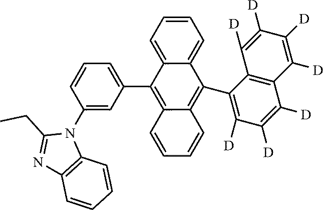 [SMILES: [2H]c1c([2H])c([2H])c2c(-c3c4ccccc4c(-c4cccc(-n5c(CC)nc6ccccc65)c4)c4ccccc34)c([2H])c([2H])c([2H])c2c1[2H]]

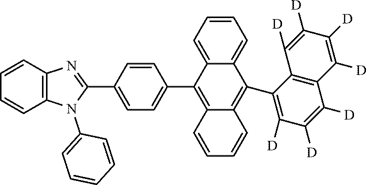 [SMILES: [2H]c1c([2H])c([2H])c2c(-c3c4ccccc4c(-c4ccc(-c5nc6ccccc6n5-c5ccccc5)cc4)c4ccccc34)c([2H])c([2H])c([2H])c2c1[2H]]

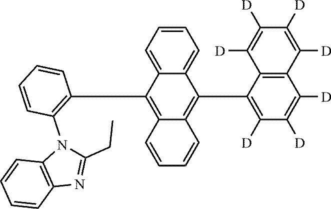 [SMILES: [2H]c1c([2H])c([2H])c2c(-c3c4ccccc4c(-c4ccccc4-n4c(CC)nc5ccccc54)c4ccccc34)c([2H])c([2H])c([2H])c2c1[2H]]

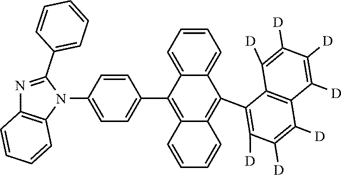 [SMILES: [2H]c1c([2H])c([2H])c2c(-c3c4ccccc4c(-c4ccc(-n5c(-c6ccccc6)nc6ccccc65)cc4)c4ccccc34)c([2H])c([2H])c([2H])c2c1[2H]]

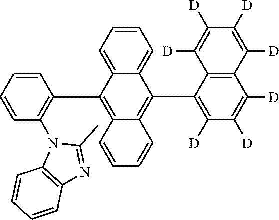 [SMILES: [2H]c1c([2H])c([2H])c2c(-c3c4ccccc4c(-c4ccccc4-n4c(C)nc5ccccc54)c4ccccc34)c([2H])c([2H])c([2H])c2c1[2H]]

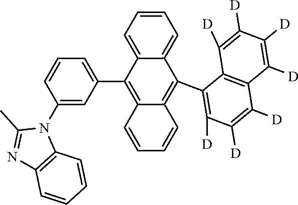 [SMILES: [2H]c1c([2H])c([2H])c2c(-c3c4ccccc4c(-c4cccc(-n5c(C)nc6ccccc65)c4)c4ccccc34)c([2H])c([2H])c([2H])c2c1[2H]]

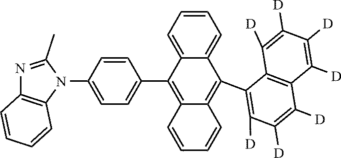 [SMILES: [2H]c1c([2H])c([2H])c2c(-c3c4ccccc4c(-c4ccc(-n5c(C)nc6ccccc65)cc4)c4ccccc34)c([2H])c([2H])c([2H])c2c1[2H]]

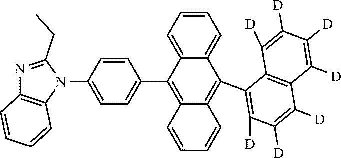 [SMILES: [2H]c1c([2H])c([2H])c2c(-c3c4ccccc4c(-c4ccc(-n5c(CC)nc6ccccc65)cc4)c4ccccc34)c([2H])c([2H])c([2H])c2c1[2H]]

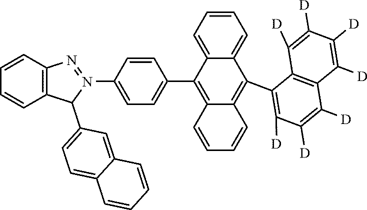

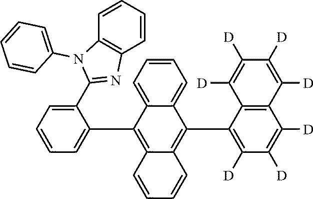 [SMILES: [2H]c1c([2H])c([2H])c2c(-c3c4ccccc4c(-c4ccccc4-c4nc5ccccc5n4-c4ccccc4)c4ccccc34)c([2H])c([2H])c([2H])c2c1[2H]]

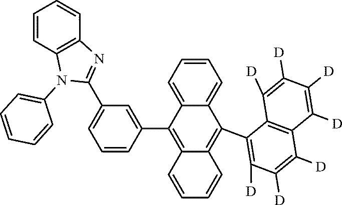 [SMILES: [2H]c1c([2H])c([2H])c2c(-c3c4ccccc4c(-c4cccc(-c5nc6ccccc6n5-c5ccccc5)c4)c4ccccc34)c([2H])c([2H])c([2H])c2c1[2H]]

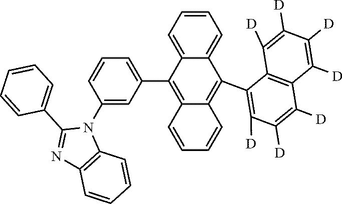 [SMILES: [2H]c1c([2H])c([2H])c2c(-c3c4ccccc4c(-c4cccc(-n5c(-c6ccccc6)nc6ccccc65)c4)c4ccccc34)c([2H])c([2H])c([2H])c2c1[2H]]

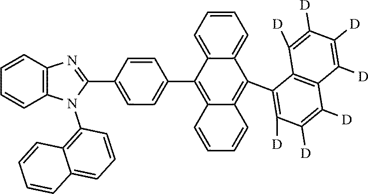 [SMILES: [2H]c1c([2H])c([2H])c2c(-c3c4ccccc4c(-c4ccc(-c5nc6ccccc6n5-c5cccc6ccccc56)cc4)c4ccccc34)c([2H])c([2H])c([2H])c2c1[2H]]

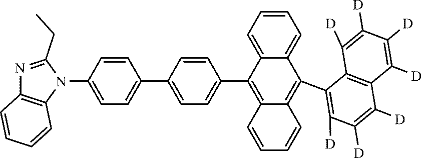 [SMILES: [2H]c1c([2H])c([2H])c2c(-c3c4ccccc4c(-c4ccc(-c5ccc(-n6c(CC)nc7ccccc76)cc5)cc4)c4ccccc34)c([2H])c([2H])c([2H])c2c1[2H]]

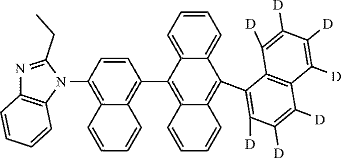 [SMILES: [2H]c1c([2H])c([2H])c2c(-c3c4ccccc4c(-c4ccc(-n5c(CC)nc6ccccc65)c5ccccc45)c4ccccc34)c([2H])c([2H])c([2H])c2c1[2H]]

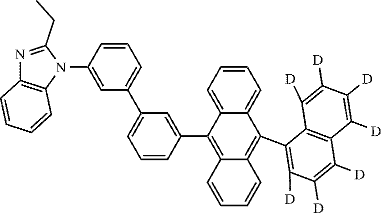 [SMILES: [2H]c1c([2H])c([2H])c2c(-c3c4ccccc4c(-c4cccc(-c5cccc(-n6c(CC)nc7ccccc76)c5)c4)c4ccccc34)c([2H])c([2H])c([2H])c2c1[2H]]

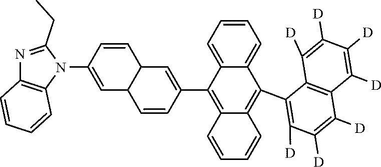 [SMILES: [2H]c1c([2H])c([2H])c2c(-c3c4ccccc4c(C4=CC5C=CC(n6c(CC)nc7ccccc76)=CC5C=C4)c4ccccc34)c([2H])c([2H])c([2H])c2c1[2H]]

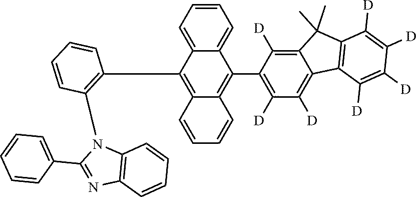 [SMILES: [2H]c1c([2H])c([2H])c2c(c1[2H])-c1c([2H])c([2H])c(-c3c4ccccc4c(-c4ccccc4-n4c(-c5ccccc5)nc5ccccc54)c4ccccc34)c([2H])c1C2(C)C]

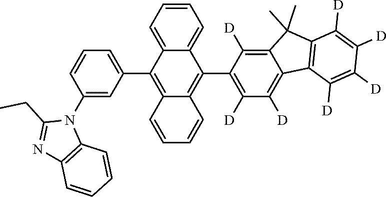 [SMILES: [2H]c1c([2H])c([2H])c2c(c1[2H])-c1c([2H])c([2H])c(-c3c4ccccc4c(-c4cccc(-n5c(CC)nc6ccccc65)c4)c4ccccc34)c([2H])c1C2(C)C]

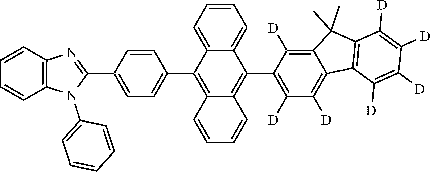 [SMILES: [2H]c1c([2H])c([2H])c2c(c1[2H])-c1c([2H])c([2H])c(-c3c4ccccc4c(-c4ccc(-c5nc6ccccc6n5-c5ccccc5)cc4)c4ccccc34)c([2H])c1C2(C)C]

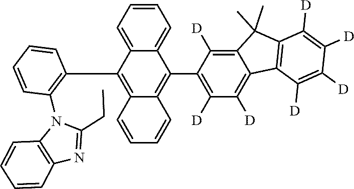 [SMILES: [2H]c1c([2H])c([2H])c2c(c1[2H])-c1c([2H])c([2H])c(-c3c4ccccc4c(-c4ccccc4-n4c(CC)nc5ccccc54)c4ccccc34)c([2H])c1C2(C)C]

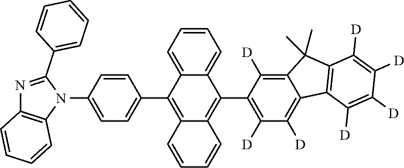 [SMILES: [2H]c1c([2H])c([2H])c2c(c1[2H])-c1c([2H])c([2H])c(-c3c4ccccc4c(-c4ccc(-n5c(-c6ccccc6)nc6ccccc65)cc4)c4ccccc34)c([2H])c1C2(C)C]

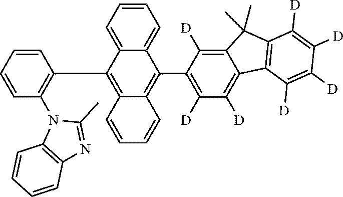 [SMILES: [2H]c1c([2H])c([2H])c2c(c1[2H])-c1c([2H])c([2H])c(-c3c4ccccc4c(-c4ccccc4-n4c(C)nc5ccccc54)c4ccccc34)c([2H])c1C2(C)C]

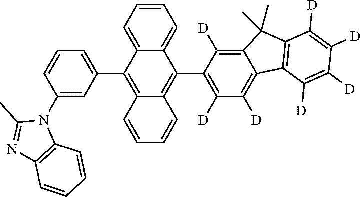 [SMILES: [2H]c1c([2H])c([2H])c2c(c1[2H])-c1c([2H])c([2H])c(-c3c4ccccc4c(-c4cccc(-n5c(C)nc6ccccc65)c4)c4ccccc34)c([2H])c1C2(C)C]

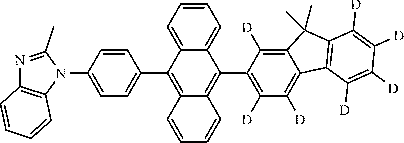 [SMILES: [2H]c1c([2H])c([2H])c2c(c1[2H])-c1c([2H])c([2H])c(-c3c4ccccc4c(-c4ccc(-n5c(C)nc6ccccc65)cc4)c4ccccc34)c([2H])c1C2(C)C]

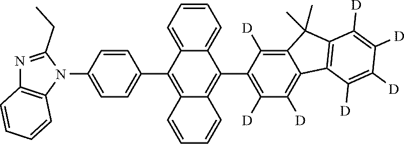 [SMILES: [2H]c1c([2H])c([2H])c2c(c1[2H])-c1c([2H])c([2H])c(-c3c4ccccc4c(-c4ccc(-n5c(CC)nc6ccccc65)cc4)c4ccccc34)c([2H])c1C2(C)C]

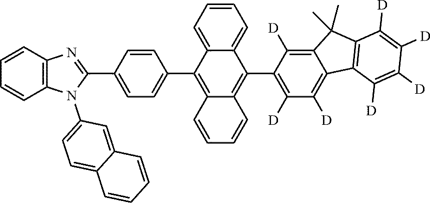 [SMILES: [2H]c1c([2H])c([2H])c2c(c1[2H])-c1c([2H])c([2H])c(-c3c4ccccc4c(-c4ccc(-c5nc6ccccc6n5-c5ccc6ccccc6c5)cc4)c4ccccc34)c([2H])c1C2(C)C]

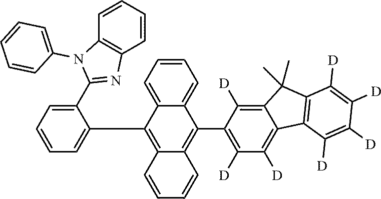 [SMILES: [2H]c1c([2H])c([2H])c2c(c1[2H])-c1c([2H])c([2H])c(-c3c4ccccc4c(-c4ccccc4-c4nc5ccccc5n4-c4ccccc4)c4ccccc34)c([2H])c1C2(C)C]

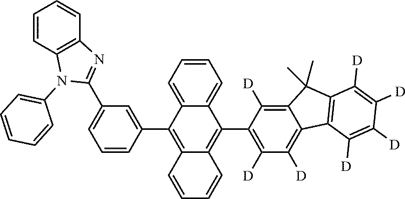 [SMILES: [2H]c1c([2H])c([2H])c2c(c1[2H])-c1c([2H])c([2H])c(-c3c4ccccc4c(-c4cccc(-c5nc6ccccc6n5-c5ccccc5)c4)c4ccccc34)c([2H])c1C2(C)C]

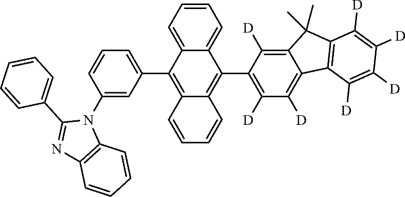 [SMILES: [2H]c1c([2H])c([2H])c2c(c1[2H])-c1c([2H])c([2H])c(-c3c4ccccc4c(-c4cccc(-n5c(-c6ccccc6)nc6ccccc65)c4)c4ccccc34)c([2H])c1C2(C)C]

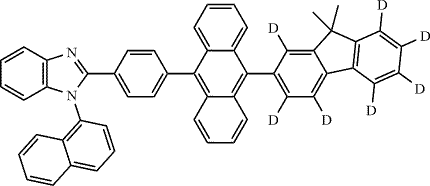 [SMILES: [2H]c1c([2H])c([2H])c2c(c1[2H])-c1c([2H])c([2H])c(-c3c4ccccc4c(-c4ccc(-c5nc6ccccc6n5-c5cccc6ccccc56)cc4)c4ccccc34)c([2H])c1C2(C)C]

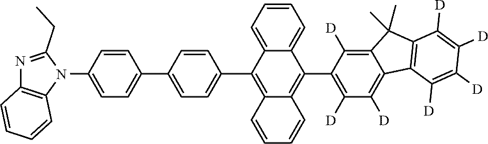 [SMILES: [2H]c1c([2H])c([2H])c2c(c1[2H])-c1c([2H])c([2H])c(-c3c4ccccc4c(-c4ccc(-c5ccc(-n6c(CC)nc7ccccc76)cc5)cc4)c4ccccc34)c([2H])c1C2(C)C]

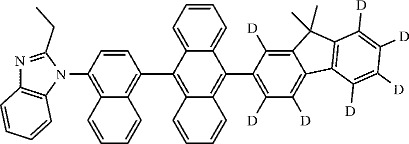 [SMILES: [2H]c1c([2H])c([2H])c2c(c1[2H])-c1c([2H])c([2H])c(-c3c4ccccc4c(-c4ccc(-n5c(CC)nc6ccccc65)c5ccccc45)c4ccccc34)c([2H])c1C2(C)C]

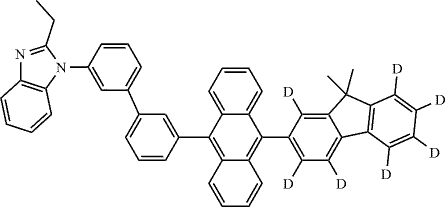 [SMILES: [2H]c1c([2H])c([2H])c2c(c1[2H])-c1c([2H])c([2H])c(-c3c4ccccc4c(-c4cccc(-c5cccc(-n6c(CC)nc7ccccc76)c5)c4)c4ccccc34)c([2H])c1C2(C)C]

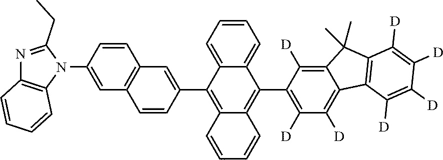 [SMILES: [2H]c1c([2H])c([2H])c2c(c1[2H])-c1c([2H])c([2H])c(-c3c4ccccc4c(-c4ccc5cc(-n6c(CC)nc7ccccc76)ccc5c4)c4ccccc34)c([2H])c1C2(C)C]

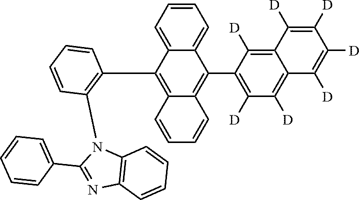 [SMILES: [2H]c1c([2H])c([2H])c2c([2H])c(-c3c4ccccc4c(-c4ccccc4-n4c(-c5ccccc5)nc5ccccc54)c4ccccc34)c([2H])c([2H])c2c1[2H]]

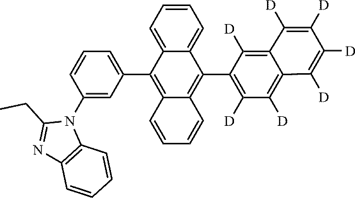 [SMILES: [2H]c1c([2H])c([2H])c2c([2H])c(-c3c4ccccc4c(-c4cccc(-n5c(CC)nc6ccccc65)c4)c4ccccc34)c([2H])c([2H])c2c1[2H]]

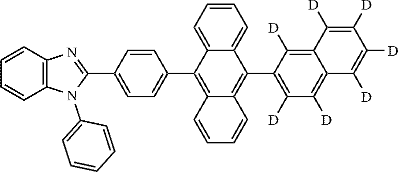 [SMILES: [2H]c1c([2H])c([2H])c2c([2H])c(-c3c4ccccc4c(-c4ccc(-c5nc6ccccc6n5-c5ccccc5)cc4)c4ccccc34)c([2H])c([2H])c2c1[2H]]

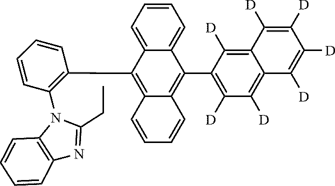 [SMILES: [2H]c1c([2H])c([2H])c2c([2H])c(-c3c4ccccc4c(-c4ccccc4-n4c(CC)nc5ccccc54)c4ccccc34)c([2H])c([2H])c2c1[2H]]

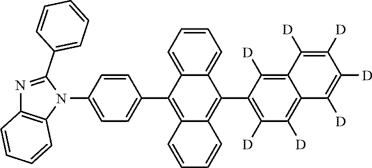 [SMILES: [2H]c1c([2H])c([2H])c2c([2H])c(-c3c4ccccc4c(-c4ccc(-n5c(-c6ccccc6)nc6ccccc65)cc4)c4ccccc34)c([2H])c([2H])c2c1[2H]]

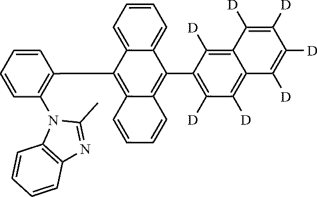 [SMILES: [2H]c1c([2H])c([2H])c2c([2H])c(-c3c4ccccc4c(-c4ccccc4-n4c(C)nc5ccccc54)c4ccccc34)c([2H])c([2H])c2c1[2H]]

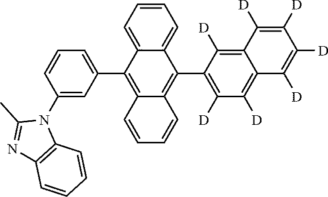 [SMILES: [2H]c1c([2H])c([2H])c2c([2H])c(-c3c4ccccc4c(-c4cccc(-n5c(C)nc6ccccc65)c4)c4ccccc34)c([2H])c([2H])c2c1[2H]]

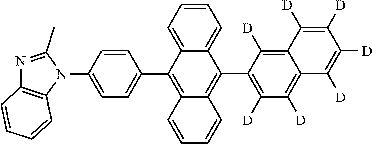 [SMILES: [2H]c1c([2H])c([2H])c2c([2H])c(-c3c4ccccc4c(-c4ccc(-n5c(C)nc6ccccc65)cc4)c4ccccc34)c([2H])c([2H])c2c1[2H]]

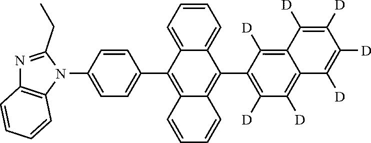 [SMILES: [2H]c1c([2H])c([2H])c2c([2H])c(-c3c4ccccc4c(-c4ccc(-n5c(CC)nc6ccccc65)cc4)c4ccccc34)c([2H])c([2H])c2c1[2H]]

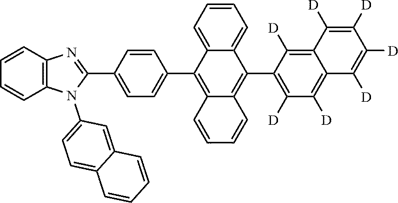 [SMILES: [2H]c1c([2H])c([2H])c2c([2H])c(-c3c4ccccc4c(-c4ccc(-c5nc6ccccc6n5-c5ccc6ccccc6c5)cc4)c4ccccc34)c([2H])c([2H])c2c1[2H]]

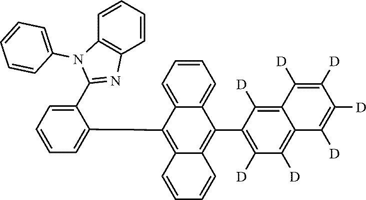 [SMILES: [2H]c1c([2H])c([2H])c2c([2H])c(-c3c4ccccc4c(-c4ccccc4-c4nc5ccccc5n4-c4ccccc4)c4ccccc34)c([2H])c([2H])c2c1[2H]]

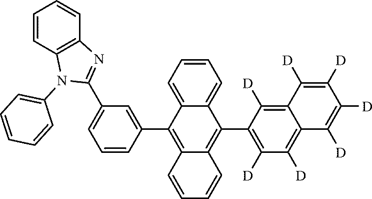 [SMILES: [2H]c1c([2H])c([2H])c2c([2H])c(-c3c4ccccc4c(-c4cccc(-c5nc6ccccc6n5-c5ccccc5)c4)c4ccccc34)c([2H])c([2H])c2c1[2H]]

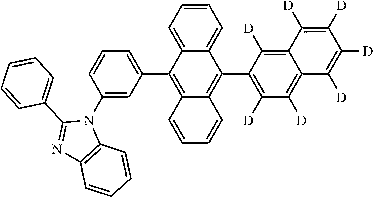 [SMILES: [2H]c1c([2H])c([2H])c2c([2H])c(-c3c4ccccc4c(-c4cccc(-n5c(-c6ccccc6)nc6ccccc65)c4)c4ccccc34)c([2H])c([2H])c2c1[2H]]

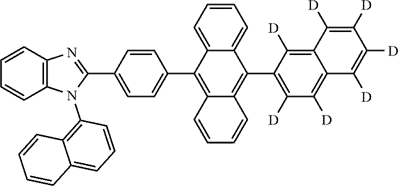 [SMILES: [2H]c1c([2H])c([2H])c2c([2H])c(-c3c4ccccc4c(-c4ccc(-c5nc6ccccc6n5-c5cccc6ccccc56)cc4)c4ccccc34)c([2H])c([2H])c2c1[2H]]

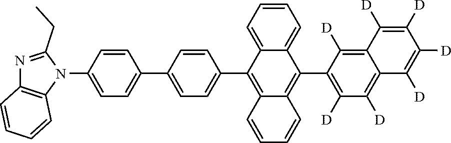 [SMILES: [2H]c1c([2H])c([2H])c2c([2H])c(-c3c4ccccc4c(-c4ccc(-c5ccc(-n6c(CC)nc7ccccc76)cc5)cc4)c4ccccc34)c([2H])c([2H])c2c1[2H]]

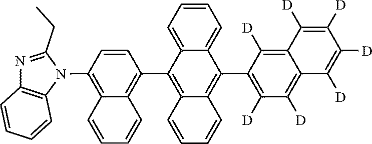 [SMILES: [2H]c1c([2H])c([2H])c2c([2H])c(-c3c4ccccc4c(-c4ccc(-n5c(CC)nc6ccccc65)c5ccccc45)c4ccccc34)c([2H])c([2H])c2c1[2H]]

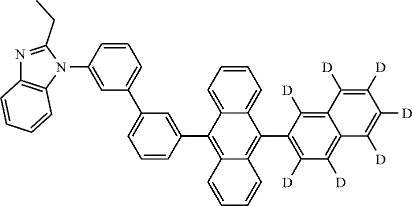 [SMILES: [2H]c1c([2H])c([2H])c2c([2H])c(-c3c4ccccc4c(-c4cccc(-c5cccc(-n6c(CC)nc7ccccc76)c5)c4)c4ccccc34)c([2H])c([2H])c2c1[2H]]

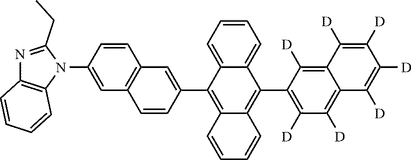 [SMILES: [2H]c1c([2H])c([2H])c2c([2H])c(-c3c4ccccc4c(-c4ccc5cc(-n6c(CC)nc7ccccc76)ccc5c4)c4ccccc34)c([2H])c([2H])c2c1[2H]]

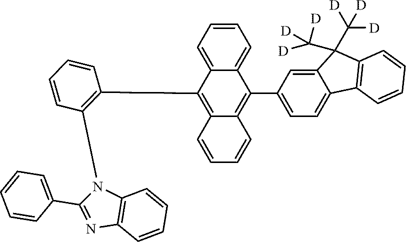 [SMILES: [2H]C([2H])([2H])C1(C([2H])([2H])[2H])c2ccccc2-c2ccc(-c3c4ccccc4c(-c4ccccc4-n4c(-c5ccccc5)nc5ccccc54)c4ccccc34)cc21]

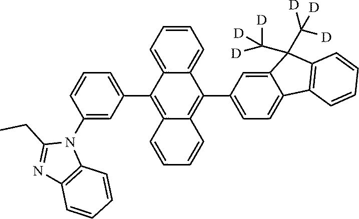 [SMILES: [2H]C([2H])([2H])C1(C([2H])([2H])[2H])c2ccccc2-c2ccc(-c3c4ccccc4c(-c4cccc(-n5c(CC)nc6ccccc65)c4)c4ccccc34)cc21]

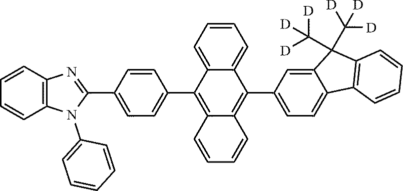 [SMILES: [2H]C([2H])([2H])C1(C([2H])([2H])[2H])c2ccccc2-c2ccc(-c3c4ccccc4c(-c4ccc(-c5nc6ccccc6n5-c5ccccc5)cc4)c4ccccc34)cc21]

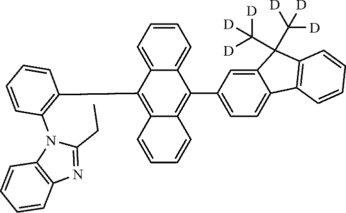 [SMILES: [2H]C([2H])([2H])C1(C([2H])([2H])[2H])c2ccccc2-c2ccc(-c3c4ccccc4c(-c4ccccc4-n4c(CC)nc5ccccc54)c4ccccc34)cc21]

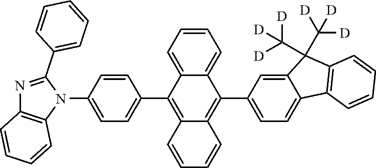 [SMILES: [2H]C([2H])([2H])C1(C([2H])([2H])[2H])c2ccccc2-c2ccc(-c3c4ccccc4c(-c4ccc(-n5c(-c6ccccc6)nc6ccccc65)cc4)c4ccccc34)cc21]

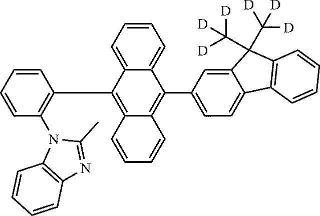 [SMILES: [2H]C([2H])([2H])C1(C([2H])([2H])[2H])c2ccccc2-c2ccc(-c3c4ccccc4c(-c4ccccc4-n4c(C)nc5ccccc54)c4ccccc34)cc21]

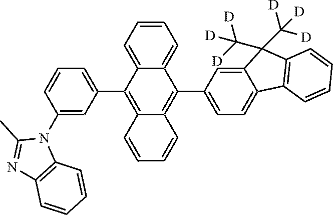 [SMILES: [2H]C([2H])([2H])C1(C([2H])([2H])[2H])c2ccccc2-c2ccc(-c3c4ccccc4c(-c4cccc(-n5c(C)nc6ccccc65)c4)c4ccccc34)cc21]

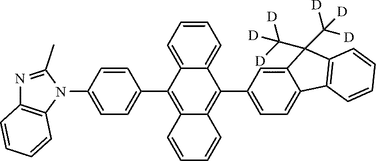 [SMILES: [2H]C([2H])([2H])C1(C([2H])([2H])[2H])c2ccccc2-c2ccc(-c3c4ccccc4c(-c4ccc(-n5c(C)nc6ccccc65)cc4)c4ccccc34)cc21]

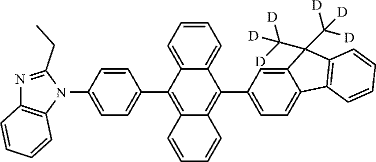 [SMILES: [2H]C([2H])([2H])C1(C([2H])([2H])[2H])c2ccccc2-c2ccc(-c3c4ccccc4c(-c4ccc(-n5c(CC)nc6ccccc65)cc4)c4ccccc34)cc21]

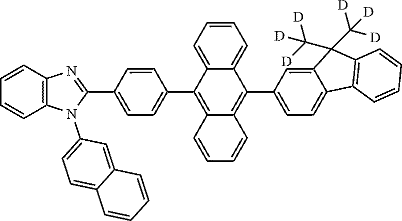 [SMILES: [2H]C([2H])([2H])C1(C([2H])([2H])[2H])c2ccccc2-c2ccc(-c3c4ccccc4c(-c4ccc(-c5nc6ccccc6n5-c5ccc6ccccc6c5)cc4)c4ccccc34)cc21]

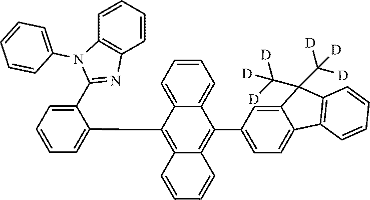 [SMILES: [2H]C([2H])([2H])C1(C([2H])([2H])[2H])c2ccccc2-c2ccc(-c3c4ccccc4c(-c4ccccc4-c4nc5ccccc5n4-c4ccccc4)c4ccccc34)cc21]

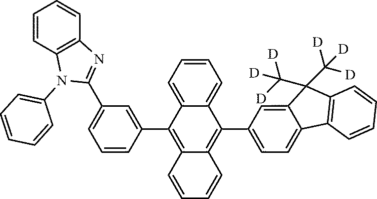 [SMILES: [2H]C([2H])([2H])C1(C([2H])([2H])[2H])c2ccccc2-c2ccc(-c3c4ccccc4c(-c4cccc(-c5nc6ccccc6n5-c5ccccc5)c4)c4ccccc34)cc21]

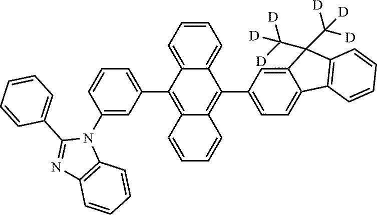 [SMILES: [2H]C([2H])([2H])C1(C([2H])([2H])[2H])c2ccccc2-c2ccc(-c3c4ccccc4c(-c4cccc(-n5c(-c6ccccc6)nc6ccccc65)c4)c4ccccc34)cc21]

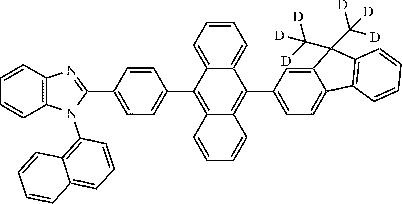 [SMILES: [2H]C([2H])([2H])C1(C([2H])([2H])[2H])c2ccccc2-c2ccc(-c3c4ccccc4c(-c4ccc(-c5nc6ccccc6n5-c5cccc6ccccc56)cc4)c4ccccc34)cc21]

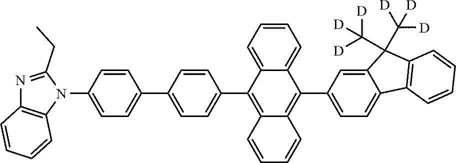 [SMILES: [2H]C([2H])([2H])C1(C([2H])([2H])[2H])c2ccccc2-c2ccc(-c3c4ccccc4c(-c4ccc(-c5ccc(-n6c(CC)nc7ccccc76)cc5)cc4)c4ccccc34)cc21]

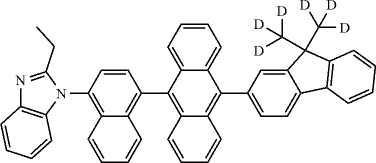 [SMILES: [2H]C([2H])([2H])C1(C([2H])([2H])[2H])c2ccccc2-c2ccc(-c3c4ccccc4c(-c4ccc(-n5c(CC)nc6ccccc65)c5ccccc45)c4ccccc34)cc21]

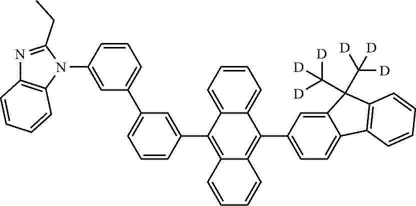 [SMILES: [2H]C([2H])([2H])C1(C([2H])([2H])[2H])c2ccccc2-c2ccc(-c3c4ccccc4c(-c4cccc(-c5cccc(-n6c(CC)nc7ccccc76)c5)c4)c4ccccc34)cc21]

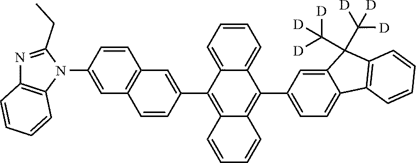 [SMILES: [2H]C([2H])([2H])C1(C([2H])([2H])[2H])c2ccccc2-c2ccc(-c3c4ccccc4c(-c4ccc5cc(-n6c(CC)nc7ccccc76)ccc5c4)c4ccccc34)cc21]

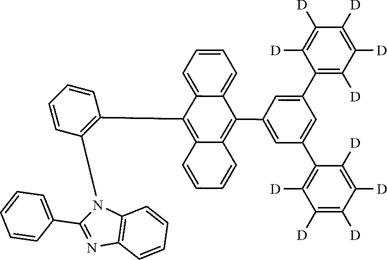 [SMILES: [2H]c1c([2H])c([2H])c(-c2cc(-c3c([2H])c([2H])c([2H])c([2H])c3[2H])cc(-c3c4ccccc4c(-c4ccccc4-n4c(-c5ccccc5)nc5ccccc54)c4ccccc34)c2)c([2H])c1[2H]]

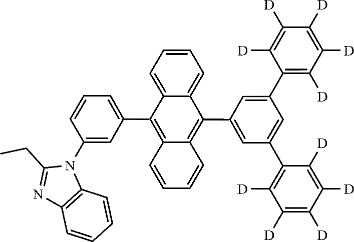 [SMILES: [2H]c1c([2H])c([2H])c(-c2cc(-c3c([2H])c([2H])c([2H])c([2H])c3[2H])cc(-c3c4ccccc4c(-c4cccc(-n5c(CC)nc6ccccc65)c4)c4ccccc34)c2)c([2H])c1[2H]]

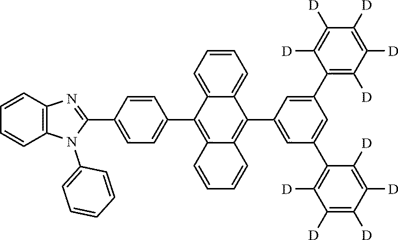 [SMILES: [2H]c1c([2H])c([2H])c(-c2cc(-c3c([2H])c([2H])c([2H])c([2H])c3[2H])cc(-c3c4ccccc4c(-c4ccc(-c5nc6ccccc6n5-c5ccccc5)cc4)c4ccccc34)c2)c([2H])c1[2H]]

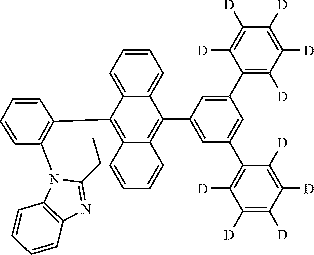 [SMILES: [2H]c1c([2H])c([2H])c(-c2cc(-c3c([2H])c([2H])c([2H])c([2H])c3[2H])cc(-c3c4ccccc4c(-c4ccccc4-n4c(CC)nc5ccccc54)c4ccccc34)c2)c([2H])c1[2H]]

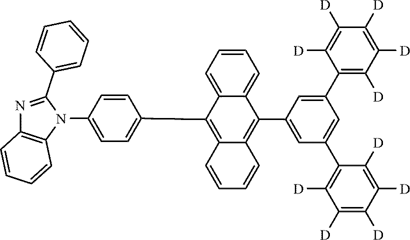 [SMILES: [2H]c1c([2H])c([2H])c(-c2cc(-c3c([2H])c([2H])c([2H])c([2H])c3[2H])cc(-c3c4ccccc4c(-c4ccc(-n5c(-c6ccccc6)nc6ccccc65)cc4)c4ccccc34)c2)c([2H])c1[2H]]

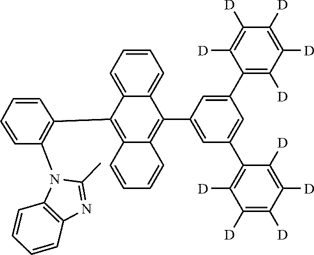 [SMILES: [2H]c1c([2H])c([2H])c(-c2cc(-c3c([2H])c([2H])c([2H])c([2H])c3[2H])cc(-c3c4ccccc4c(-c4ccccc4-n4c(C)nc5ccccc54)c4ccccc34)c2)c([2H])c1[2H]]

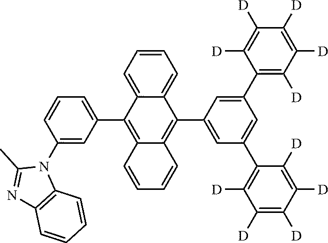 [SMILES: [2H]c1c([2H])c([2H])c(-c2cc(-c3c([2H])c([2H])c([2H])c([2H])c3[2H])cc(-c3c4ccccc4c(-c4cccc(-n5c(C)nc6ccccc65)c4)c4ccccc34)c2)c([2H])c1[2H]]

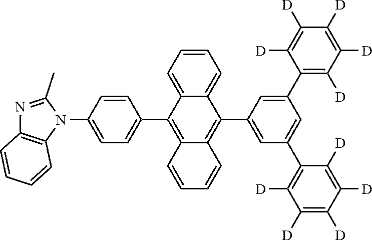 [SMILES: [2H]c1c([2H])c([2H])c(-c2cc(-c3c([2H])c([2H])c([2H])c([2H])c3[2H])cc(-c3c4ccccc4c(-c4ccc(-n5c(C)nc6ccccc65)cc4)c4ccccc34)c2)c([2H])c1[2H]]

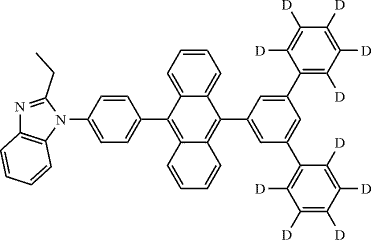 [SMILES: [2H]c1c([2H])c([2H])c(-c2cc(-c3c([2H])c([2H])c([2H])c([2H])c3[2H])cc(-c3c4ccccc4c(-c4ccc(-n5c(CC)nc6ccccc65)cc4)c4ccccc34)c2)c([2H])c1[2H]]

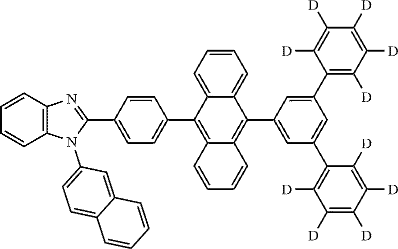 [SMILES: [2H]c1c([2H])c([2H])c(-c2cc(-c3c([2H])c([2H])c([2H])c([2H])c3[2H])cc(-c3c4ccccc4c(-c4ccc(-c5nc6ccccc6n5-c5ccc6ccccc6c5)cc4)c4ccccc34)c2)c([2H])c1[2H]]

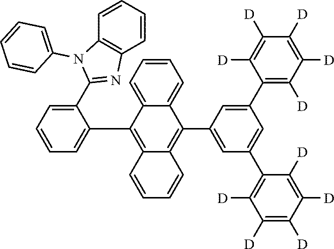 [SMILES: [2H]c1c([2H])c([2H])c(-c2cc(-c3c([2H])c([2H])c([2H])c([2H])c3[2H])cc(-c3c4ccccc4c(-c4ccccc4-c4nc5ccccc5n4-c4ccccc4)c4ccccc34)c2)c([2H])c1[2H]]

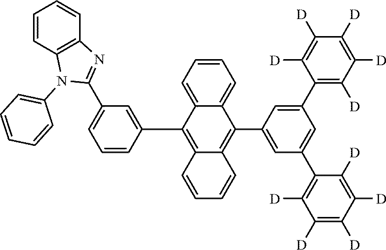 [SMILES: [2H]c1c([2H])c([2H])c(-c2cc(-c3c([2H])c([2H])c([2H])c([2H])c3[2H])cc(-c3c4ccccc4c(-c4cccc(-c5nc6ccccc6n5-c5ccccc5)c4)c4ccccc34)c2)c([2H])c1[2H]]

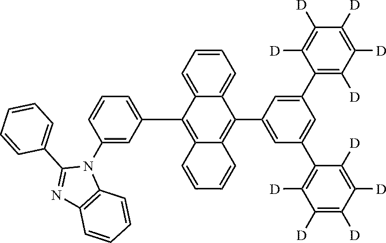 [SMILES: [2H]c1c([2H])c([2H])c(-c2cc(-c3c([2H])c([2H])c([2H])c([2H])c3[2H])cc(-c3c4ccccc4c(-c4cccc(-n5c(-c6ccccc6)nc6ccccc65)c4)c4ccccc34)c2)c([2H])c1[2H]]

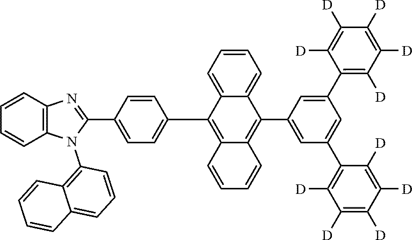 [SMILES: [2H]c1c([2H])c([2H])c(-c2cc(-c3c([2H])c([2H])c([2H])c([2H])c3[2H])cc(-c3c4ccccc4c(-c4ccc(-c5nc6ccccc6n5-c5cccc6ccccc56)cc4)c4ccccc34)c2)c([2H])c1[2H]]

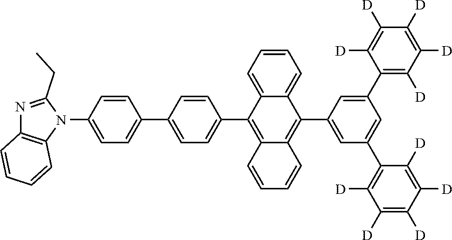 [SMILES: [2H]c1c([2H])c([2H])c(-c2cc(-c3c([2H])c([2H])c([2H])c([2H])c3[2H])cc(-c3c4ccccc4c(-c4ccc(-c5ccc(-n6c(CC)nc7ccccc76)cc5)cc4)c4ccccc34)c2)c([2H])c1[2H]]

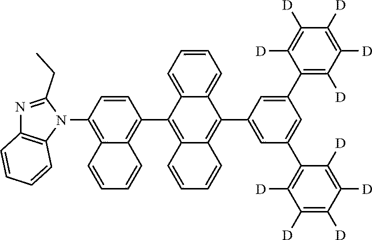 [SMILES: [2H]c1c([2H])c([2H])c(-c2cc(-c3c([2H])c([2H])c([2H])c([2H])c3[2H])cc(-c3c4ccccc4c(-c4ccc(-n5c(CC)nc6ccccc65)c5ccccc45)c4ccccc34)c2)c([2H])c1[2H]]

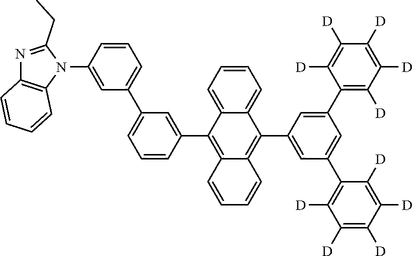 [SMILES: [2H]c1c([2H])c([2H])c(-c2cc(-c3c([2H])c([2H])c([2H])c([2H])c3[2H])cc(-c3c4ccccc4c(-c4cccc(-c5cccc(-n6c(CC)nc7ccccc76)c5)c4)c4ccccc34)c2)c([2H])c1[2H]]

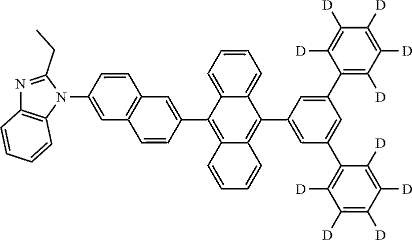 [SMILES: [2H]c1c([2H])c([2H])c(-c2cc(-c3c([2H])c([2H])c([2H])c([2H])c3[2H])cc(-c3c4ccccc4c(-c4ccc5cc(-n6c(CC)nc7ccccc76)ccc5c4)c4ccccc34)c2)c([2H])c1[2H]]

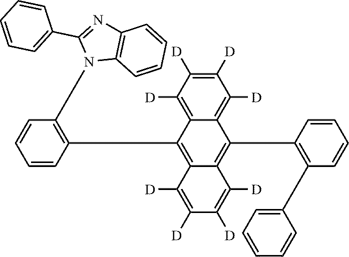 [SMILES: [2H]c1c([2H])c([2H])c2c(-c3ccccc3-n3c(-c4ccccc4)nc4ccccc43)c3c([2H])c([2H])c([2H])c([2H])c3c(-c3ccccc3-c3ccccc3)c2c1[2H]]

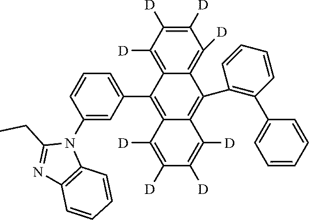 [SMILES: [2H]c1c([2H])c([2H])c2c(-c3ccccc3-c3ccccc3)c3c([2H])c([2H])c([2H])c([2H])c3c(-c3cccc(-n4c(CC)nc5ccccc54)c3)c2c1[2H]]

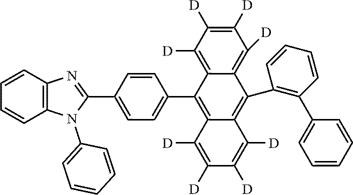 [SMILES: [2H]c1c([2H])c([2H])c2c(-c3ccccc3-c3ccccc3)c3c([2H])c([2H])c([2H])c([2H])c3c(-c3ccc(-c4nc5ccccc5n4-c4ccccc4)cc3)c2c1[2H]]

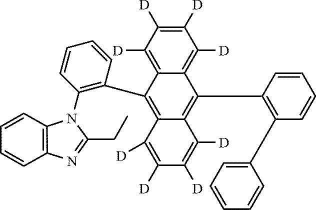 [SMILES: [2H]c1c([2H])c([2H])c2c(-c3ccccc3-n3c(CC)nc4ccccc43)c3c([2H])c([2H])c([2H])c([2H])c3c(-c3ccccc3-c3ccccc3)c2c1[2H]]

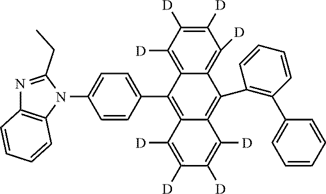 [SMILES: [2H]c1c([2H])c([2H])c2c(-c3ccccc3-c3ccccc3)c3c([2H])c([2H])c([2H])c([2H])c3c(-c3ccc(-n4c(CC)nc5ccccc54)cc3)c2c1[2H]]

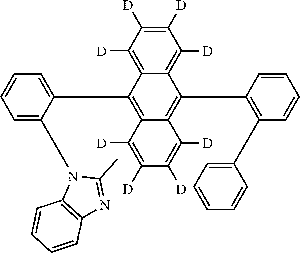 [SMILES: [2H]c1c([2H])c([2H])c2c(-c3ccccc3-n3c(C)nc4ccccc43)c3c([2H])c([2H])c([2H])c([2H])c3c(-c3ccccc3-c3ccccc3)c2c1[2H]]

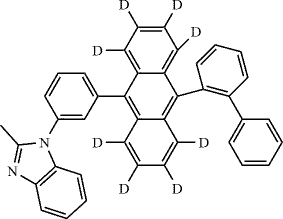 [SMILES: [2H]c1c([2H])c([2H])c2c(-c3ccccc3-c3ccccc3)c3c([2H])c([2H])c([2H])c([2H])c3c(-c3cccc(-n4c(C)nc5ccccc54)c3)c2c1[2H]]

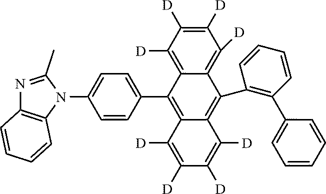 [SMILES: [2H]c1c([2H])c([2H])c2c(-c3ccccc3-c3ccccc3)c3c([2H])c([2H])c([2H])c([2H])c3c(-c3ccc(-n4c(C)nc5ccccc54)cc3)c2c1[2H]]

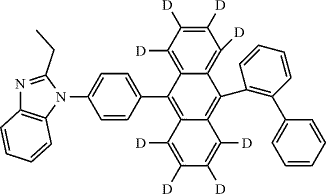 [SMILES: [2H]c1c([2H])c([2H])c2c(-c3ccccc3-c3ccccc3)c3c([2H])c([2H])c([2H])c([2H])c3c(-c3ccc(-n4c(CC)nc5ccccc54)cc3)c2c1[2H]]

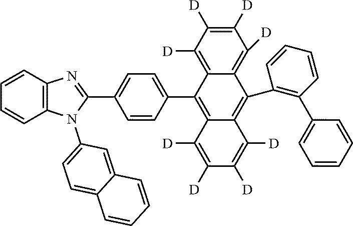 [SMILES: [2H]c1c([2H])c([2H])c2c(-c3ccccc3-c3ccccc3)c3c([2H])c([2H])c([2H])c([2H])c3c(-c3ccc(-c4nc5ccccc5n4-c4ccc5ccccc5c4)cc3)c2c1[2H]]

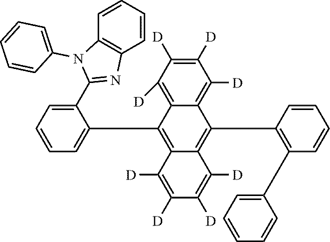 [SMILES: [2H]c1c([2H])c([2H])c2c(-c3ccccc3-c3nc4ccccc4n3-c3ccccc3)c3c([2H])c([2H])c([2H])c([2H])c3c(-c3ccccc3-c3ccccc3)c2c1[2H]]

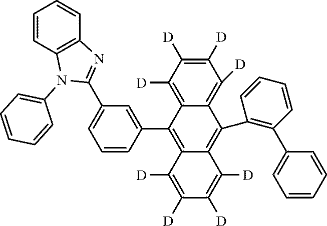 [SMILES: [2H]c1c([2H])c([2H])c2c(-c3ccccc3-c3ccccc3)c3c([2H])c([2H])c([2H])c([2H])c3c(-c3cccc(-c4nc5ccccc5n4-c4ccccc4)c3)c2c1[2H]]

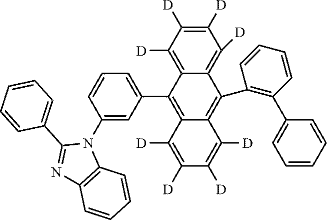 [SMILES: [2H]c1c([2H])c([2H])c2c(-c3ccccc3-c3ccccc3)c3c([2H])c([2H])c([2H])c([2H])c3c(-c3cccc(-n4c(-c5ccccc5)nc5ccccc54)c3)c2c1[2H]]

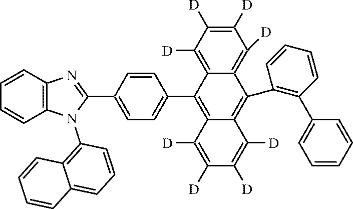 [SMILES: [2H]c1c([2H])c([2H])c2c(-c3ccccc3-c3ccccc3)c3c([2H])c([2H])c([2H])c([2H])c3c(-c3ccc(-c4nc5ccccc5n4-c4cccc5ccccc45)cc3)c2c1[2H]]

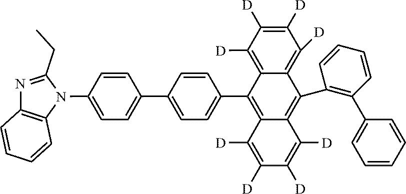 [SMILES: [2H]c1c([2H])c([2H])c2c(-c3ccccc3-c3ccccc3)c3c([2H])c([2H])c([2H])c([2H])c3c(-c3ccc(-c4ccc(-n5c(CC)nc6ccccc65)cc4)cc3)c2c1[2H]]

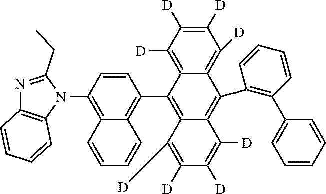 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccccc3-c3ccccc3)c2c1[2H]]

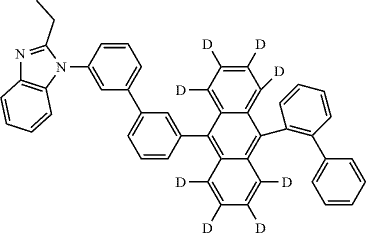 [SMILES: [2H]c1c([2H])c([2H])c2c(-c3ccccc3-c3ccccc3)c3c([2H])c([2H])c([2H])c([2H])c3c(-c3cccc(-c4cccc(-n5c(CC)nc6ccccc65)c4)c3)c2c1[2H]]

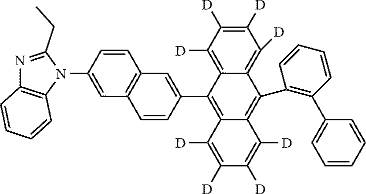 [SMILES: [2H]c1c([2H])c([2H])c2c(-c3ccccc3-c3ccccc3)c3c([2H])c([2H])c([2H])c([2H])c3c(-c3ccc4cc(-n5c(CC)nc6ccccc65)ccc4c3)c2c1[2H]]

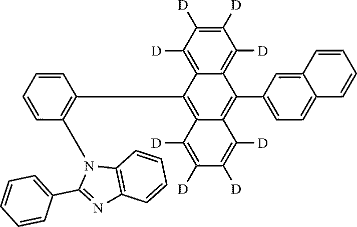 [SMILES: [2H]c1c([2H])c([2H])c2c(-c3ccccc3-n3c(-c4ccccc4)nc4ccccc43)c3c([2H])c([2H])c([2H])c([2H])c3c(-c3ccc4ccccc4c3)c2c1[2H]]

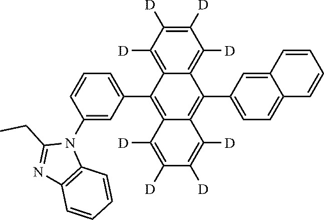 [SMILES: [2H]c1c([2H])c([2H])c2c(-c3ccc4ccccc4c3)c3c([2H])c([2H])c([2H])c([2H])c3c(-c3cccc(-n4c(CC)nc5ccccc54)c3)c2c1[2H]]

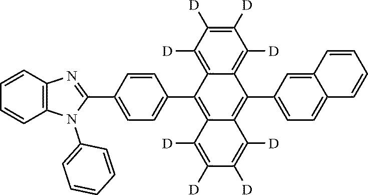 [SMILES: [2H]c1c([2H])c([2H])c2c(-c3ccc4ccccc4c3)c3c([2H])c([2H])c([2H])c([2H])c3c(-c3ccc(-c4nc5ccccc5n4-c4ccccc4)cc3)c2c1[2H]]

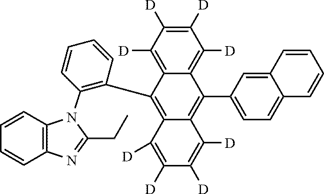 [SMILES: [2H]c1c([2H])c([2H])c2c(-c3ccccc3-n3c(CC)nc4ccccc43)c3c([2H])c([2H])c([2H])c([2H])c3c(-c3ccc4ccccc4c3)c2c1[2H]]

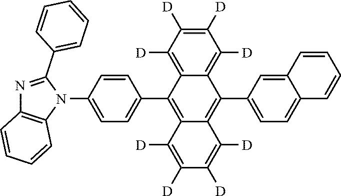 [SMILES: [2H]c1c([2H])c([2H])c2c(-c3ccc4ccccc4c3)c3c([2H])c([2H])c([2H])c([2H])c3c(-c3ccc(-n4c(-c5ccccc5)nc5ccccc54)cc3)c2c1[2H]]

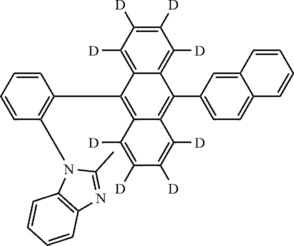 [SMILES: [2H]c1c([2H])c([2H])c2c(-c3ccccc3-n3c(C)nc4ccccc43)c3c([2H])c([2H])c([2H])c([2H])c3c(-c3ccc4ccccc4c3)c2c1[2H]]

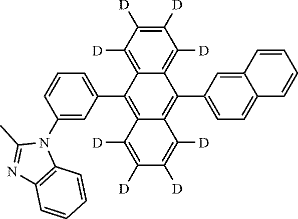 [SMILES: [2H]c1c([2H])c([2H])c2c(-c3ccc4ccccc4c3)c3c([2H])c([2H])c([2H])c([2H])c3c(-c3cccc(-n4c(C)nc5ccccc54)c3)c2c1[2H]]

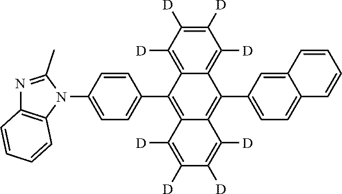 [SMILES: [2H]c1c([2H])c([2H])c2c(-c3ccc4ccccc4c3)c3c([2H])c([2H])c([2H])c([2H])c3c(-c3ccc(-n4c(C)nc5ccccc54)cc3)c2c1[2H]]

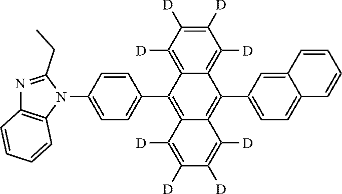 [SMILES: [2H]c1c([2H])c([2H])c2c(-c3ccc4ccccc4c3)c3c([2H])c([2H])c([2H])c([2H])c3c(-c3ccc(-n4c(CC)nc5ccccc54)cc3)c2c1[2H]]

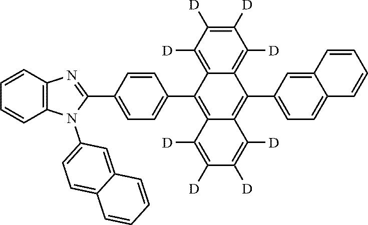 [SMILES: [2H]c1c([2H])c([2H])c2c(-c3ccc4ccccc4c3)c3c([2H])c([2H])c([2H])c([2H])c3c(-c3ccc(-c4nc5ccccc5n4-c4ccc5ccccc5c4)cc3)c2c1[2H]]

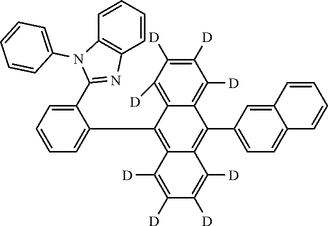 [SMILES: [2H]c1c([2H])c([2H])c2c(-c3ccccc3-c3nc4ccccc4n3-c3ccccc3)c3c([2H])c([2H])c([2H])c([2H])c3c(-c3ccc4ccccc4c3)c2c1[2H]]

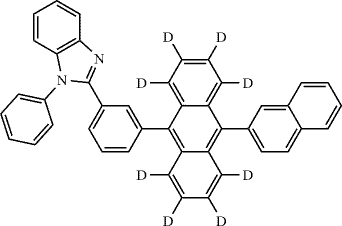 [SMILES: [2H]c1c([2H])c([2H])c2c(-c3ccc4ccccc4c3)c3c([2H])c([2H])c([2H])c([2H])c3c(-c3cccc(-c4nc5ccccc5n4-c4ccccc4)c3)c2c1[2H]]

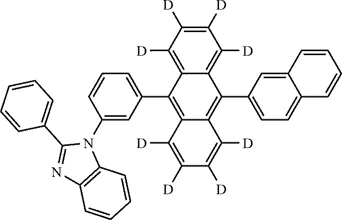 [SMILES: [2H]c1c([2H])c([2H])c2c(-c3ccc4ccccc4c3)c3c([2H])c([2H])c([2H])c([2H])c3c(-c3cccc(-n4c(-c5ccccc5)nc5ccccc54)c3)c2c1[2H]]

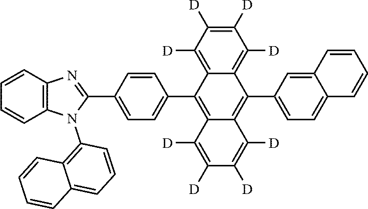 [SMILES: [2H]c1c([2H])c([2H])c2c(-c3ccc4ccccc4c3)c3c([2H])c([2H])c([2H])c([2H])c3c(-c3ccc(-c4nc5ccccc5n4-c4cccc5ccccc45)cc3)c2c1[2H]]

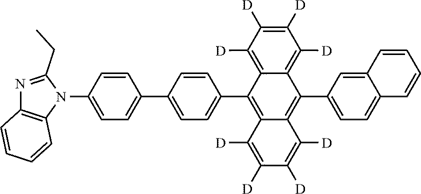 [SMILES: [2H]c1c([2H])c([2H])c2c(-c3ccc4ccccc4c3)c3c([2H])c([2H])c([2H])c([2H])c3c(-c3ccc(-c4ccc(-n5c(CC)nc6ccccc65)cc4)cc3)c2c1[2H]]

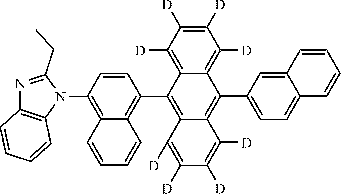 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccc4ccccc4c3)c2c1[2H]]

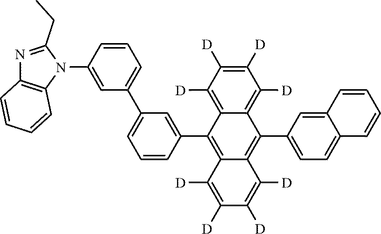 [SMILES: [2H]c1c([2H])c([2H])c2c(-c3ccc4ccccc4c3)c3c([2H])c([2H])c([2H])c([2H])c3c(-c3cccc(-c4cccc(-n5c(CC)nc6ccccc65)c4)c3)c2c1[2H]]

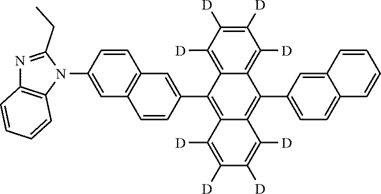 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3ccc4ccccc4c3)c2c1[2H]]

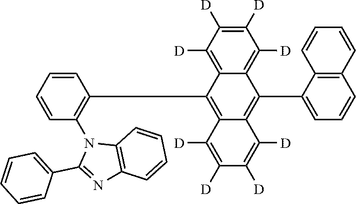 [SMILES: [2H]c1c([2H])c([2H])c2c(-c3cccc4ccccc34)c3c([2H])c([2H])c([2H])c([2H])c3c(-c3ccccc3-n3c(-c4ccccc4)nc4ccccc43)c2c1[2H]]

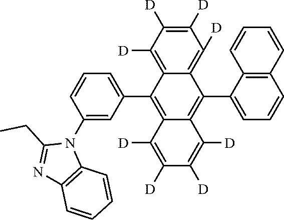 [SMILES: [2H]c1c([2H])c([2H])c2c(-c3cccc4ccccc34)c3c([2H])c([2H])c([2H])c([2H])c3c(-c3cccc(-n4c(CC)nc5ccccc54)c3)c2c1[2H]]

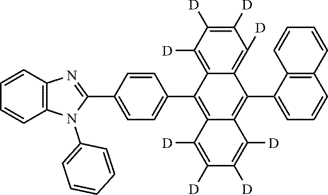 [SMILES: [2H]c1c([2H])c([2H])c2c(-c3cccc4ccccc34)c3c([2H])c([2H])c([2H])c([2H])c3c(-c3ccc(-c4nc5ccccc5n4-c4ccccc4)cc3)c2c1[2H]]

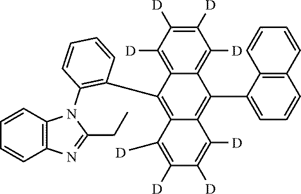 [SMILES: [2H]c1c([2H])c([2H])c2c(-c3cccc4ccccc34)c3c([2H])c([2H])c([2H])c([2H])c3c(-c3ccccc3-n3c(CC)nc4ccccc43)c2c1[2H]]

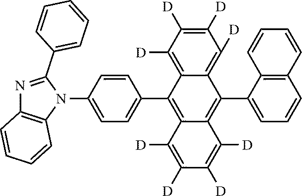 [SMILES: [2H]c1c([2H])c([2H])c2c(-c3cccc4ccccc34)c3c([2H])c([2H])c([2H])c([2H])c3c(-c3ccc(-n4c(-c5ccccc5)nc5ccccc54)cc3)c2c1[2H]]

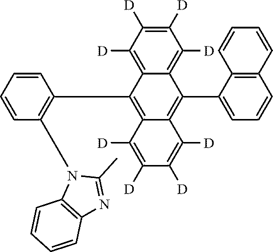 [SMILES: [2H]c1c([2H])c([2H])c2c(-c3cccc4ccccc34)c3c([2H])c([2H])c([2H])c([2H])c3c(-c3ccccc3-n3c(C)nc4ccccc43)c2c1[2H]]

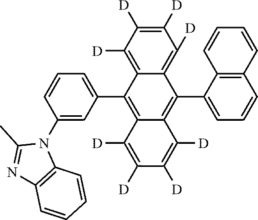 [SMILES: [2H]c1c([2H])c([2H])c2c(-c3cccc4ccccc34)c3c([2H])c([2H])c([2H])c([2H])c3c(-c3cccc(-n4c(C)nc5ccccc54)c3)c2c1[2H]]

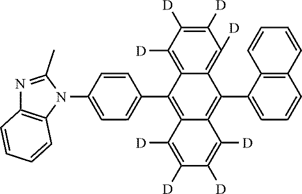 [SMILES: [2H]c1c([2H])c([2H])c2c(-c3cccc4ccccc34)c3c([2H])c([2H])c([2H])c([2H])c3c(-c3ccc(-n4c(C)nc5ccccc54)cc3)c2c1[2H]]

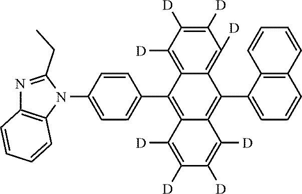 [SMILES: [2H]c1c([2H])c([2H])c2c(-c3cccc4ccccc34)c3c([2H])c([2H])c([2H])c([2H])c3c(-c3ccc(-n4c(CC)nc5ccccc54)cc3)c2c1[2H]]

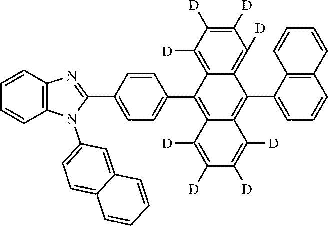 [SMILES: [2H]c1c([2H])c([2H])c2c(-c3cccc4ccccc34)c3c([2H])c([2H])c([2H])c([2H])c3c(-c3ccc(-c4nc5ccccc5n4-c4ccc5ccccc5c4)cc3)c2c1[2H]]

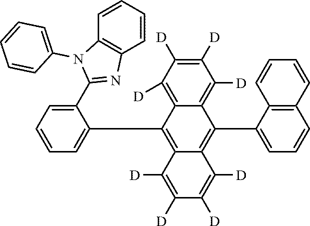 [SMILES: [2H]c1c([2H])c([2H])c2c(-c3cccc4ccccc34)c3c([2H])c([2H])c([2H])c([2H])c3c(-c3ccccc3-c3nc4ccccc4n3-c3ccccc3)c2c1[2H]]

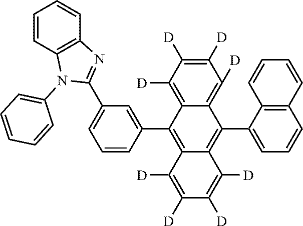 [SMILES: [2H]c1c([2H])c([2H])c2c(-c3cccc4ccccc34)c3c([2H])c([2H])c([2H])c([2H])c3c(-c3cccc(-c4nc5ccccc5n4-c4ccccc4)c3)c2c1[2H]]

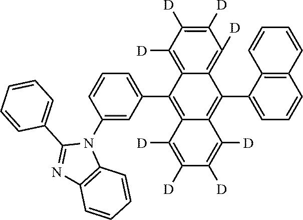 [SMILES: [2H]c1c([2H])c([2H])c2c(-c3cccc4ccccc34)c3c([2H])c([2H])c([2H])c([2H])c3c(-c3cccc(-n4c(-c5ccccc5)nc5ccccc54)c3)c2c1[2H]]

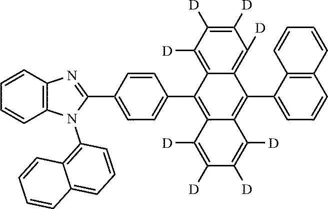 [SMILES: [2H]c1c([2H])c([2H])c2c(-c3cccc4ccccc34)c3c([2H])c([2H])c([2H])c([2H])c3c(-c3ccc(-c4nc5ccccc5n4-c4cccc5ccccc45)cc3)c2c1[2H]]

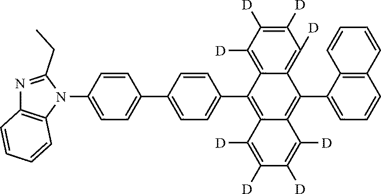 [SMILES: [2H]c1c([2H])c([2H])c2c(-c3cccc4ccccc34)c3c([2H])c([2H])c([2H])c([2H])c3c(-c3ccc(-c4ccc(-n5c(CC)nc6ccccc65)cc4)cc3)c2c1[2H]]

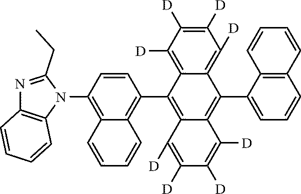 [SMILES: [2H]c1c([2H])c([2H])c2c(-c3ccc(-n4c(CC)nc5ccccc54)c4ccccc34)c3c([2H])c([2H])c([2H])c([2H])c3c(-c3cccc4ccccc34)c2c1[2H]]

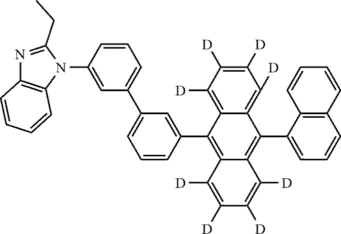 [SMILES: [2H]c1c([2H])c([2H])c2c(-c3cccc4ccccc34)c3c([2H])c([2H])c([2H])c([2H])c3c(-c3cccc(-c4cccc(-n5c(CC)nc6ccccc65)c4)c3)c2c1[2H]]

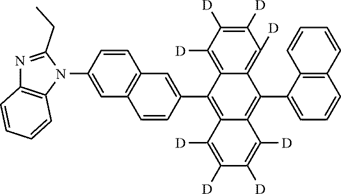 [SMILES: [2H]c1c([2H])c([2H])c2c(-c3cccc4ccccc34)c3c([2H])c([2H])c([2H])c([2H])c3c(-c3ccc4cc(-n5c(CC)nc6ccccc65)ccc4c3)c2c1[2H]]

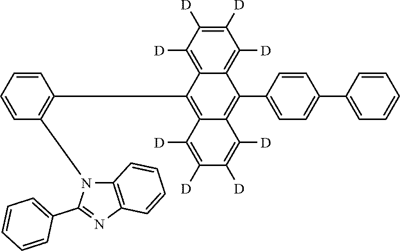 [SMILES: [2H]c1c([2H])c([2H])c2c(-c3ccccc3-n3c(-c4ccccc4)nc4ccccc43)c3c([2H])c([2H])c([2H])c([2H])c3c(-c3ccc(-c4ccccc4)cc3)c2c1[2H]]

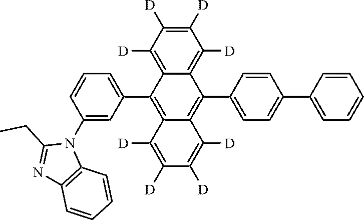 [SMILES: [2H]c1c([2H])c([2H])c2c(-c3cccc(-n4c(CC)nc5ccccc54)c3)c3c([2H])c([2H])c([2H])c([2H])c3c(-c3ccc(-c4ccccc4)cc3)c2c1[2H]]

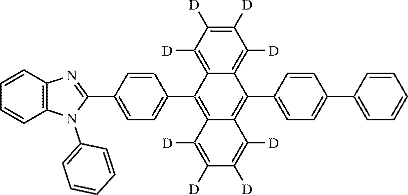 [SMILES: [2H]c1c([2H])c([2H])c2c(-c3ccc(-c4nc5ccccc5n4-c4ccccc4)cc3)c3c([2H])c([2H])c([2H])c([2H])c3c(-c3ccc(-c4ccccc4)cc3)c2c1[2H]]

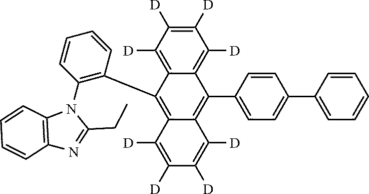 [SMILES: [2H]c1c([2H])c([2H])c2c(-c3ccccc3-n3c(CC)nc4ccccc43)c3c([2H])c([2H])c([2H])c([2H])c3c(-c3ccc(-c4ccccc4)cc3)c2c1[2H]]

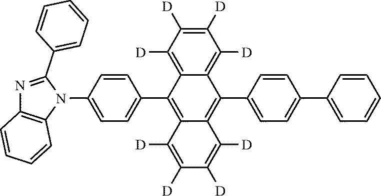 [SMILES: [2H]c1c([2H])c([2H])c2c(-c3ccc(-n4c(-c5ccccc5)nc5ccccc54)cc3)c3c([2H])c([2H])c([2H])c([2H])c3c(-c3ccc(-c4ccccc4)cc3)c2c1[2H]]

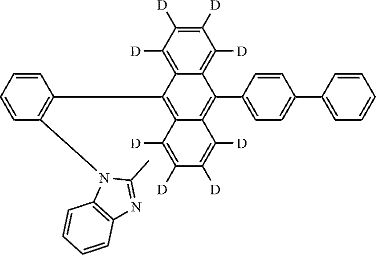 [SMILES: [2H]c1c([2H])c([2H])c2c(-c3ccccc3-n3c(C)nc4ccccc43)c3c([2H])c([2H])c([2H])c([2H])c3c(-c3ccc(-c4ccccc4)cc3)c2c1[2H]]

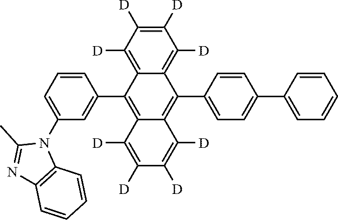 [SMILES: [2H]c1c([2H])c([2H])c2c(-c3cccc(-n4c(C)nc5ccccc54)c3)c3c([2H])c([2H])c([2H])c([2H])c3c(-c3ccc(-c4ccccc4)cc3)c2c1[2H]]

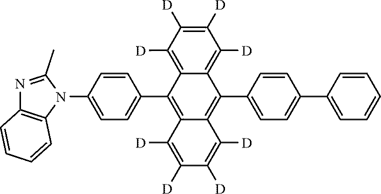 [SMILES: [2H]c1c([2H])c([2H])c2c(-c3ccc(-n4c(C)nc5ccccc54)cc3)c3c([2H])c([2H])c([2H])c([2H])c3c(-c3ccc(-c4ccccc4)cc3)c2c1[2H]]

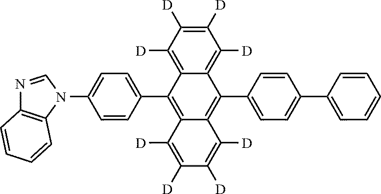 [SMILES: [2H]c1c([2H])c([2H])c2c(-c3ccc(-n4cnc5ccccc54)cc3)c3c([2H])c([2H])c([2H])c([2H])c3c(-c3ccc(-c4ccccc4)cc3)c2c1[2H]]

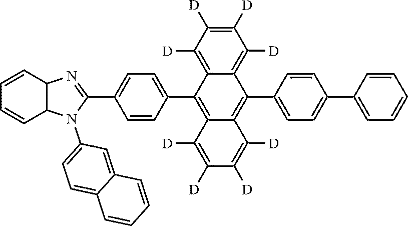 [SMILES: [2H]c1c([2H])c([2H])c2c(-c3ccc(-c4ccccc4)cc3)c3c([2H])c([2H])c([2H])c([2H])c3c(-c3ccc(C4=NC5C=CC=CC5N4c4ccc5ccccc5c4)cc3)c2c1[2H]]

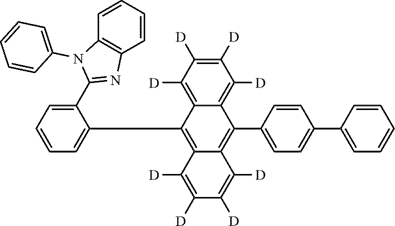 [SMILES: [2H]c1c([2H])c([2H])c2c(-c3ccccc3-c3nc4ccccc4n3-c3ccccc3)c3c([2H])c([2H])c([2H])c([2H])c3c(-c3ccc(-c4ccccc4)cc3)c2c1[2H]]

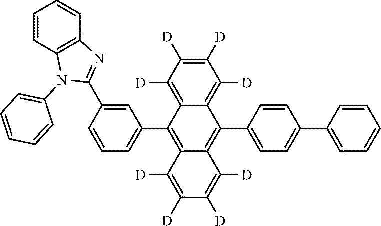 [SMILES: [2H]c1c([2H])c([2H])c2c(-c3cccc(-c4nc5ccccc5n4-c4ccccc4)c3)c3c([2H])c([2H])c([2H])c([2H])c3c(-c3ccc(-c4ccccc4)cc3)c2c1[2H]]

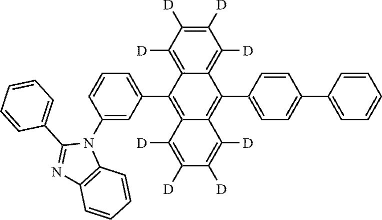 [SMILES: [2H]c1c([2H])c([2H])c2c(-c3cccc(-n4c(-c5ccccc5)nc5ccccc54)c3)c3c([2H])c([2H])c([2H])c([2H])c3c(-c3ccc(-c4ccccc4)cc3)c2c1[2H]]

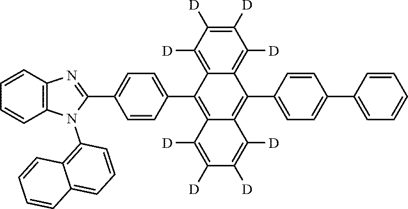 [SMILES: [2H]c1c([2H])c([2H])c2c(-c3ccc(-c4nc5ccccc5n4-c4cccc5ccccc45)cc3)c3c([2H])c([2H])c([2H])c([2H])c3c(-c3ccc(-c4ccccc4)cc3)c2c1[2H]]

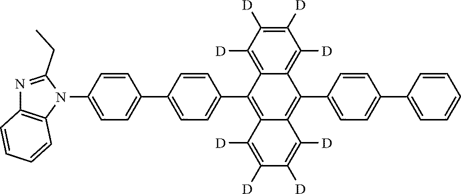 [SMILES: [2H]c1c([2H])c([2H])c2c(-c3ccc(-c4ccc(-n5c(CC)nc6ccccc65)cc4)cc3)c3c([2H])c([2H])c([2H])c([2H])c3c(-c3ccc(-c4ccccc4)cc3)c2c1[2H]]

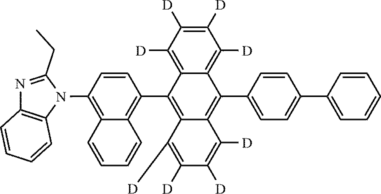 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccc(-c4ccccc4)cc3)c2c1[2H]]

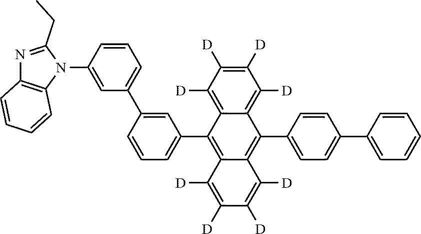 [SMILES: [2H]c1c([2H])c([2H])c2c(-c3cccc(-c4cccc(-n5c(CC)nc6ccccc65)c4)c3)c3c([2H])c([2H])c([2H])c([2H])c3c(-c3ccc(-c4ccccc4)cc3)c2c1[2H]]

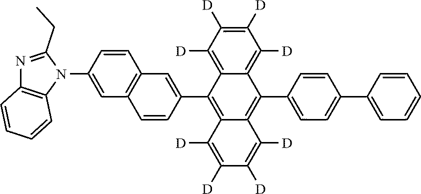 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3ccc(-c4ccccc4)cc3)c2c1[2H]]

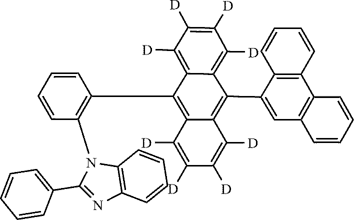 [SMILES: [2H]c1c([2H])c([2H])c2c(-c3cc4ccccc4c4ccccc34)c3c([2H])c([2H])c([2H])c([2H])c3c(-c3ccccc3-n3c(-c4ccccc4)nc4ccccc43)c2c1[2H]]

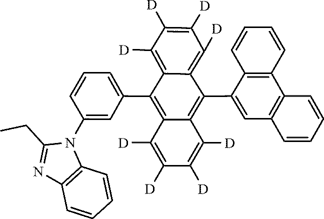 [SMILES: [2H]c1c([2H])c([2H])c2c(-c3cc4ccccc4c4ccccc34)c3c([2H])c([2H])c([2H])c([2H])c3c(-c3cccc(-n4c(CC)nc5ccccc54)c3)c2c1[2H]]

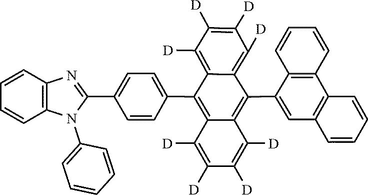 [SMILES: [2H]c1c([2H])c([2H])c2c(-c3cc4ccccc4c4ccccc34)c3c([2H])c([2H])c([2H])c([2H])c3c(-c3ccc(-c4nc5ccccc5n4-c4ccccc4)cc3)c2c1[2H]]

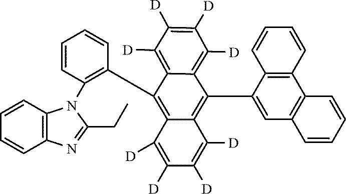 [SMILES: [2H]c1c([2H])c([2H])c2c(-c3cc4ccccc4c4ccccc34)c3c([2H])c([2H])c([2H])c([2H])c3c(-c3ccccc3-n3c(CC)nc4ccccc43)c2c1[2H]]

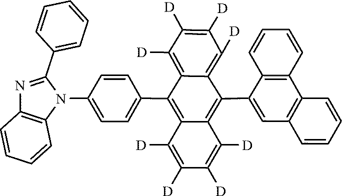 [SMILES: [2H]c1c([2H])c([2H])c2c(-c3cc4ccccc4c4ccccc34)c3c([2H])c([2H])c([2H])c([2H])c3c(-c3ccc(-n4c(-c5ccccc5)nc5ccccc54)cc3)c2c1[2H]]

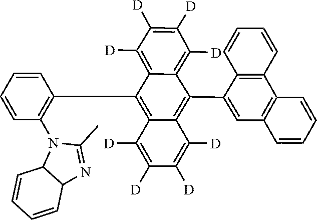 [SMILES: [2H]c1c([2H])c([2H])c2c(-c3cc4ccccc4c4ccccc34)c3c([2H])c([2H])c([2H])c([2H])c3c(-c3ccccc3N3C(C)=NC4C=CC=CC43)c2c1[2H]]

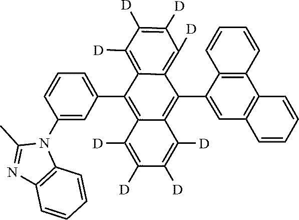 [SMILES: [2H]c1c([2H])c([2H])c2c(-c3cc4ccccc4c4ccccc34)c3c([2H])c([2H])c([2H])c([2H])c3c(-c3cccc(-n4c(C)nc5ccccc54)c3)c2c1[2H]]

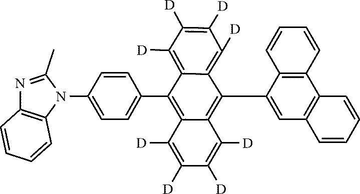 [SMILES: [2H]c1c([2H])c([2H])c2c(-c3cc4ccccc4c4ccccc34)c3c([2H])c([2H])c([2H])c([2H])c3c(-c3ccc(-n4c(C)nc5ccccc54)cc3)c2c1[2H]]

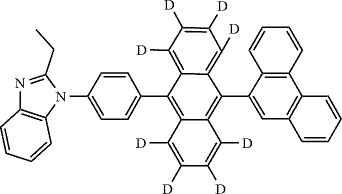 [SMILES: [2H]c1c([2H])c([2H])c2c(-c3cc4ccccc4c4ccccc34)c3c([2H])c([2H])c([2H])c([2H])c3c(-c3ccc(-n4c(CC)nc5ccccc54)cc3)c2c1[2H]]

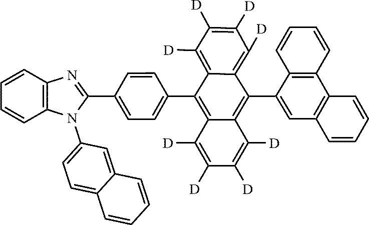 [SMILES: [2H]c1c([2H])c([2H])c2c(-c3cc4ccccc4c4ccccc34)c3c([2H])c([2H])c([2H])c([2H])c3c(-c3ccc(-c4nc5ccccc5n4-c4ccc5ccccc5c4)cc3)c2c1[2H]]

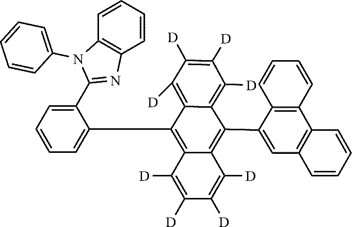 [SMILES: [2H]c1c([2H])c([2H])c2c(-c3cc4ccccc4c4ccccc34)c3c([2H])c([2H])c([2H])c([2H])c3c(-c3ccccc3-c3nc4ccccc4n3-c3ccccc3)c2c1[2H]]

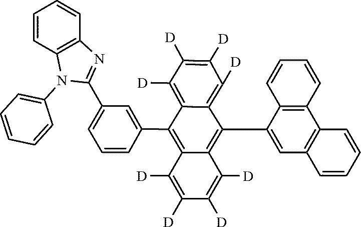 [SMILES: [2H]c1c([2H])c([2H])c2c(-c3cc4ccccc4c4ccccc34)c3c([2H])c([2H])c([2H])c([2H])c3c(-c3cccc(-c4nc5ccccc5n4-c4ccccc4)c3)c2c1[2H]]

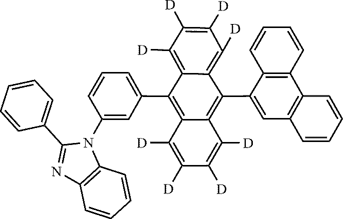 [SMILES: [2H]c1c([2H])c([2H])c2c(-c3cc4ccccc4c4ccccc34)c3c([2H])c([2H])c([2H])c([2H])c3c(-c3cccc(-n4c(-c5ccccc5)nc5ccccc54)c3)c2c1[2H]]

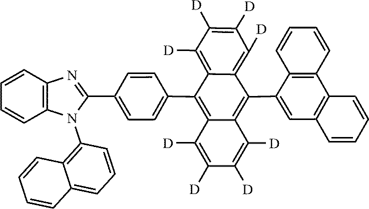 [SMILES: [2H]c1c([2H])c([2H])c2c(-c3cc4ccccc4c4ccccc34)c3c([2H])c([2H])c([2H])c([2H])c3c(-c3ccc(-c4nc5ccccc5n4-c4cccc5ccccc45)cc3)c2c1[2H]]

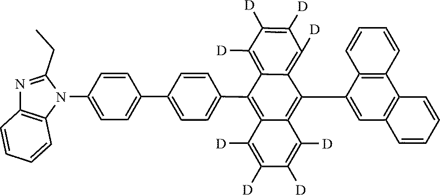 [SMILES: [2H]c1c([2H])c([2H])c2c(-c3cc4ccccc4c4ccccc34)c3c([2H])c([2H])c([2H])c([2H])c3c(-c3ccc(-c4ccc(-n5c(CC)nc6ccccc65)cc4)cc3)c2c1[2H]]

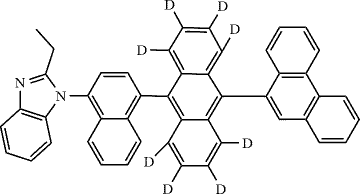 [SMILES: [2H]c1c([2H])c([2H])c2c(-c3cc4ccccc4c4ccccc34)c3c([2H])c([2H])c([2H])c([2H])c3c(-c3ccc(-n4c(CC)nc5ccccc54)c4ccccc34)c2c1[2H]]

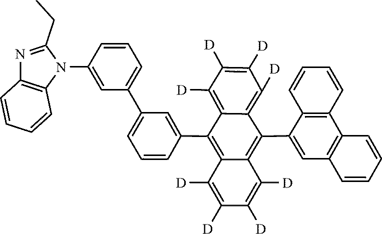 [SMILES: [2H]c1c([2H])c([2H])c2c(-c3cc4ccccc4c4ccccc34)c3c([2H])c([2H])c([2H])c([2H])c3c(-c3cccc(-c4cccc(-n5c(CC)nc6ccccc65)c4)c3)c2c1[2H]]

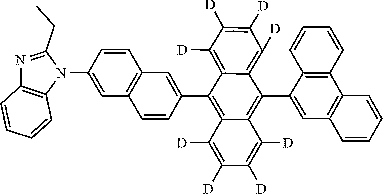 [SMILES: [2H]c1c([2H])c([2H])c2c(-c3cc4ccccc4c4ccccc34)c3c([2H])c([2H])c([2H])c([2H])c3c(-c3ccc4cc(-n5c(CC)nc6ccccc65)ccc4c3)c2c1[2H]]

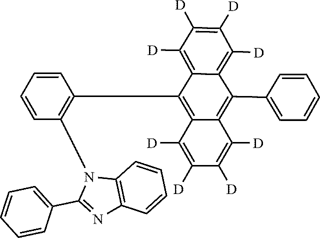 [SMILES: [2H]c1c([2H])c([2H])c2c(-c3ccccc3-n3c(-c4ccccc4)nc4ccccc43)c3c([2H])c([2H])c([2H])c([2H])c3c(-c3ccccc3)c2c1[2H]]

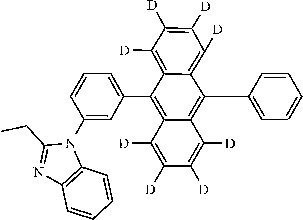 [SMILES: [2H]c1c([2H])c([2H])c2c(-c3cccc(-n4c(CC)nc5ccccc54)c3)c3c([2H])c([2H])c([2H])c([2H])c3c(-c3ccccc3)c2c1[2H]]

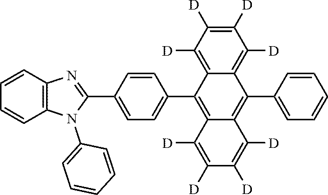 [SMILES: [2H]c1c([2H])c([2H])c2c(-c3ccc(-c4nc5ccccc5n4-c4ccccc4)cc3)c3c([2H])c([2H])c([2H])c([2H])c3c(-c3ccccc3)c2c1[2H]]

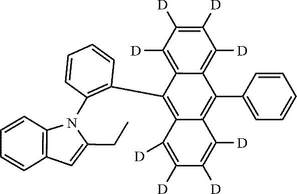 [SMILES: [2H]c1c([2H])c([2H])c2c(-c3ccccc3-n3c(CC)cc4ccccc43)c3c([2H])c([2H])c([2H])c([2H])c3c(-c3ccccc3)c2c1[2H]]

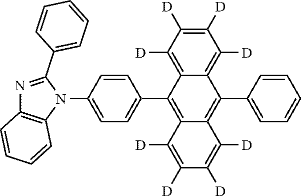 [SMILES: [2H]c1c([2H])c([2H])c2c(-c3ccc(-n4c(-c5ccccc5)nc5ccccc54)cc3)c3c([2H])c([2H])c([2H])c([2H])c3c(-c3ccccc3)c2c1[2H]]

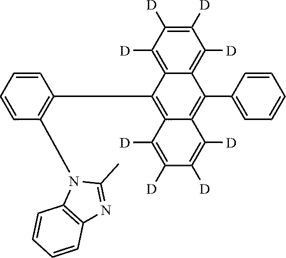 [SMILES: [2H]c1c([2H])c([2H])c2c(-c3ccccc3-n3c(C)nc4ccccc43)c3c([2H])c([2H])c([2H])c([2H])c3c(-c3ccccc3)c2c1[2H]]

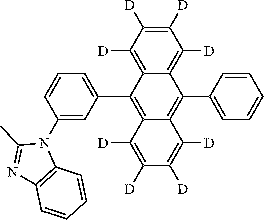 [SMILES: [2H]c1c([2H])c([2H])c2c(-c3cccc(-n4c(C)nc5ccccc54)c3)c3c([2H])c([2H])c([2H])c([2H])c3c(-c3ccccc3)c2c1[2H]]

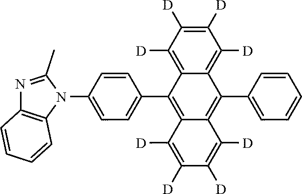 [SMILES: [2H]c1c([2H])c([2H])c2c(-c3ccc(-n4c(C)nc5ccccc54)cc3)c3c([2H])c([2H])c([2H])c([2H])c3c(-c3ccccc3)c2c1[2H]]

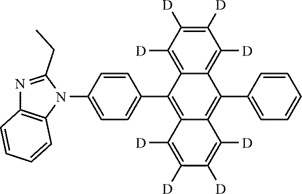 [SMILES: [2H]c1c([2H])c([2H])c2c(-c3ccc(-n4c(CC)nc5ccccc54)cc3)c3c([2H])c([2H])c([2H])c([2H])c3c(-c3ccccc3)c2c1[2H]]

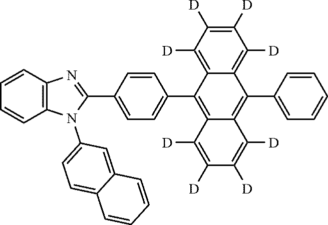 [SMILES: [2H]c1c([2H])c([2H])c2c(-c3ccc(-c4nc5ccccc5n4-c4ccc5ccccc5c4)cc3)c3c([2H])c([2H])c([2H])c([2H])c3c(-c3ccccc3)c2c1[2H]]

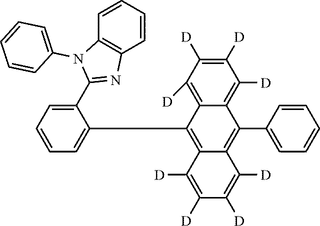 [SMILES: [2H]c1c([2H])c([2H])c2c(-c3ccccc3-c3nc4ccccc4n3-c3ccccc3)c3c([2H])c([2H])c([2H])c([2H])c3c(-c3ccccc3)c2c1[2H]]

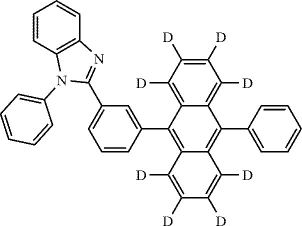 [SMILES: [2H]c1c([2H])c([2H])c2c(-c3cccc(-c4nc5ccccc5n4-c4ccccc4)c3)c3c([2H])c([2H])c([2H])c([2H])c3c(-c3ccccc3)c2c1[2H]]

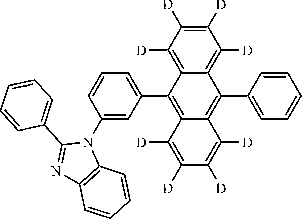 [SMILES: [2H]c1c([2H])c([2H])c2c(-c3cccc(-n4c(-c5ccccc5)nc5ccccc54)c3)c3c([2H])c([2H])c([2H])c([2H])c3c(-c3ccccc3)c2c1[2H]]

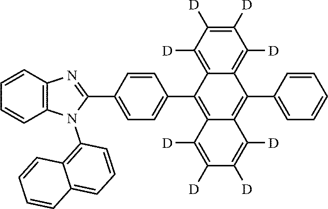 [SMILES: [2H]c1c([2H])c([2H])c2c(-c3ccc(-c4nc5ccccc5n4-c4cccc5ccccc45)cc3)c3c([2H])c([2H])c([2H])c([2H])c3c(-c3ccccc3)c2c1[2H]]

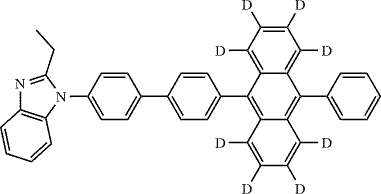 [SMILES: [2H]c1c([2H])c([2H])c2c(-c3ccc(-c4ccc(-n5c(CC)nc6ccccc65)cc4)cc3)c3c([2H])c([2H])c([2H])c([2H])c3c(-c3ccccc3)c2c1[2H]]

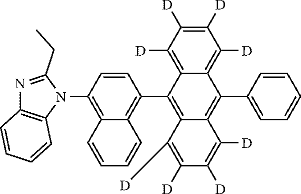 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccccc3)c2c1[2H]]

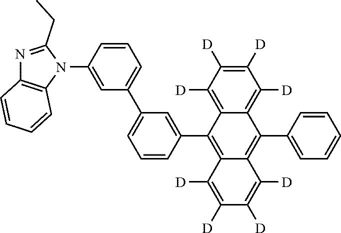 [SMILES: [2H]c1c([2H])c([2H])c2c(-c3cccc(-c4cccc(-n5c(CC)nc6ccccc65)c4)c3)c3c([2H])c([2H])c([2H])c([2H])c3c(-c3ccccc3)c2c1[2H]]

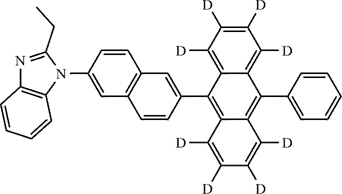 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3ccccc3)c2c1[2H]]

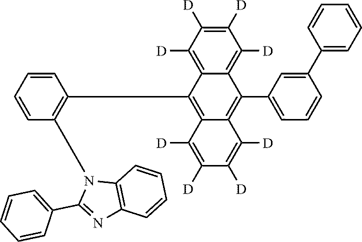 [SMILES: [2H]c1c([2H])c([2H])c2c(-c3ccccc3-n3c(-c4ccccc4)nc4ccccc43)c3c([2H])c([2H])c([2H])c([2H])c3c(-c3cccc(-c4ccccc4)c3)c2c1[2H]]

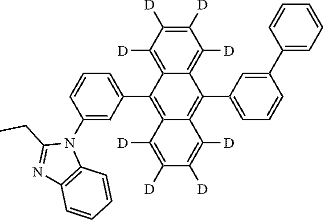 [SMILES: [2H]c1c([2H])c([2H])c2c(-c3cccc(-n4c(CC)nc5ccccc54)c3)c3c([2H])c([2H])c([2H])c([2H])c3c(-c3cccc(-c4ccccc4)c3)c2c1[2H]]

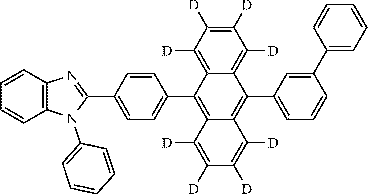 [SMILES: [2H]c1c([2H])c([2H])c2c(-c3cccc(-c4ccccc4)c3)c3c([2H])c([2H])c([2H])c([2H])c3c(-c3ccc(-c4nc5ccccc5n4-c4ccccc4)cc3)c2c1[2H]]

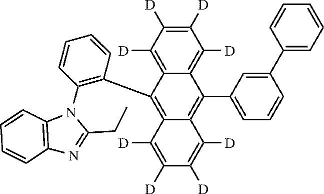 [SMILES: [2H]c1c([2H])c([2H])c2c(-c3ccccc3-n3c(CC)nc4ccccc43)c3c([2H])c([2H])c([2H])c([2H])c3c(-c3cccc(-c4ccccc4)c3)c2c1[2H]]

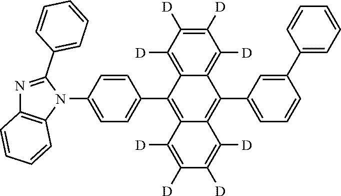 [SMILES: [2H]c1c([2H])c([2H])c2c(-c3cccc(-c4ccccc4)c3)c3c([2H])c([2H])c([2H])c([2H])c3c(-c3ccc(-n4c(-c5ccccc5)nc5ccccc54)cc3)c2c1[2H]]

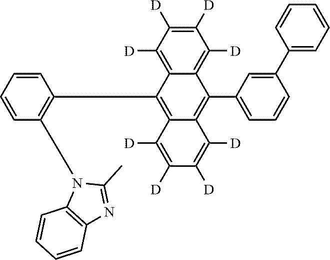 [SMILES: [2H]c1c([2H])c([2H])c2c(-c3ccccc3-n3c(C)nc4ccccc43)c3c([2H])c([2H])c([2H])c([2H])c3c(-c3cccc(-c4ccccc4)c3)c2c1[2H]]

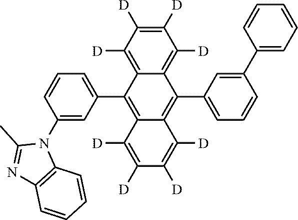 [SMILES: [2H]c1c([2H])c([2H])c2c(-c3cccc(-n4c(C)nc5ccccc54)c3)c3c([2H])c([2H])c([2H])c([2H])c3c(-c3cccc(-c4ccccc4)c3)c2c1[2H]]

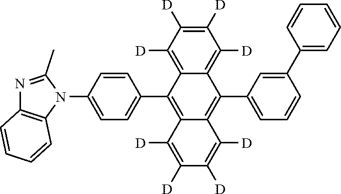 [SMILES: [2H]c1c([2H])c([2H])c2c(-c3cccc(-c4ccccc4)c3)c3c([2H])c([2H])c([2H])c([2H])c3c(-c3ccc(-n4c(C)nc5ccccc54)cc3)c2c1[2H]]

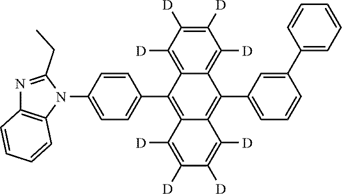 [SMILES: [2H]c1c([2H])c([2H])c2c(-c3cccc(-c4ccccc4)c3)c3c([2H])c([2H])c([2H])c([2H])c3c(-c3ccc(-n4c(CC)nc5ccccc54)cc3)c2c1[2H]]

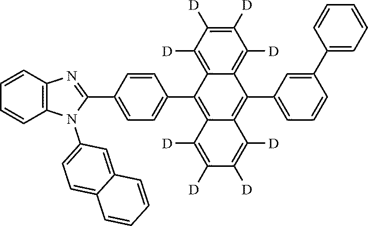 [SMILES: [2H]c1c([2H])c([2H])c2c(-c3cccc(-c4ccccc4)c3)c3c([2H])c([2H])c([2H])c([2H])c3c(-c3ccc(-c4nc5ccccc5n4-c4ccc5ccccc5c4)cc3)c2c1[2H]]

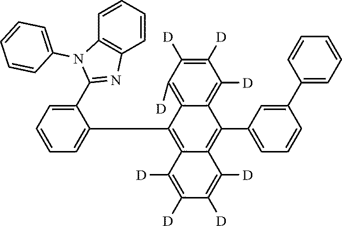 [SMILES: [2H]c1c([2H])c([2H])c2c(-c3ccccc3-c3nc4ccccc4n3-c3ccccc3)c3c([2H])c([2H])c([2H])c([2H])c3c(-c3cccc(-c4ccccc4)c3)c2c1[2H]]

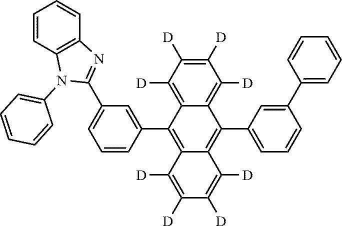 [SMILES: [2H]c1c([2H])c([2H])c2c(-c3cccc(-c4nc5ccccc5n4-c4ccccc4)c3)c3c([2H])c([2H])c([2H])c([2H])c3c(-c3cccc(-c4ccccc4)c3)c2c1[2H]]

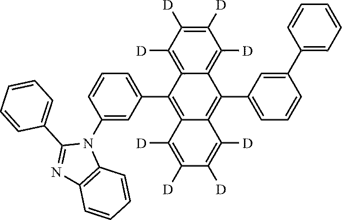 [SMILES: [2H]c1c([2H])c([2H])c2c(-c3cccc(-n4c(-c5ccccc5)nc5ccccc54)c3)c3c([2H])c([2H])c([2H])c([2H])c3c(-c3cccc(-c4ccccc4)c3)c2c1[2H]]

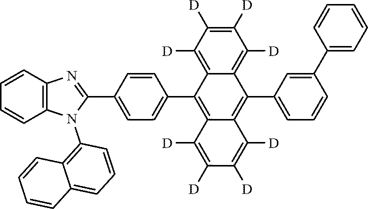 [SMILES: [2H]c1c([2H])c([2H])c2c(-c3cccc(-c4ccccc4)c3)c3c([2H])c([2H])c([2H])c([2H])c3c(-c3ccc(-c4nc5ccccc5n4-c4cccc5ccccc45)cc3)c2c1[2H]]

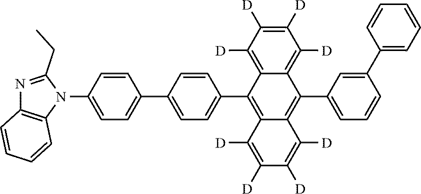 [SMILES: [2H]c1c([2H])c([2H])c2c(-c3cccc(-c4ccccc4)c3)c3c([2H])c([2H])c([2H])c([2H])c3c(-c3ccc(-c4ccc(-n5c(CC)nc6ccccc65)cc4)cc3)c2c1[2H]]

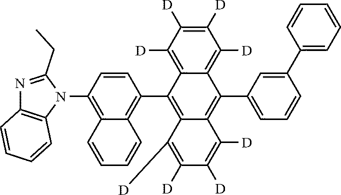 [SMILES: [2H]c1c([2H])c([2H])c2c(-c3ccc(-n4c(CC)nc5ccccc54)c4ccccc34)c3c([2H])c([2H])c([2H])c([2H])c3c(-c3cccc(-c4ccccc4)c3)c2c1[2H]]

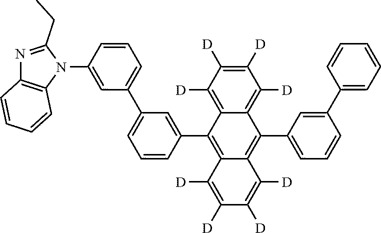 [SMILES: [2H]c1c([2H])c([2H])c2c(-c3cccc(-c4cccc(-n5c(CC)nc6ccccc65)c4)c3)c3c([2H])c([2H])c([2H])c([2H])c3c(-c3cccc(-c4ccccc4)c3)c2c1[2H]]

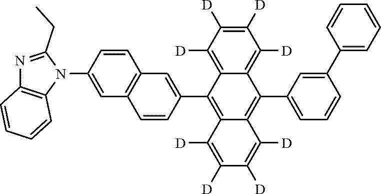 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3cccc(-c4ccccc4)c3)c2c1[2H]]

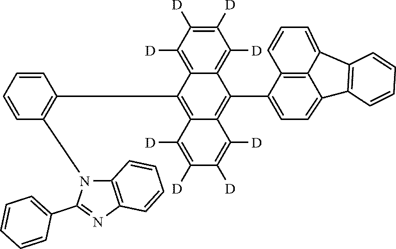 [SMILES: [2H]c1c([2H])c([2H])c2c(-c3ccc4c5c(cccc35)-c3ccccc3-4)c3c([2H])c([2H])c([2H])c([2H])c3c(-c3ccccc3-n3c(-c4ccccc4)nc4ccccc43)c2c1[2H]]

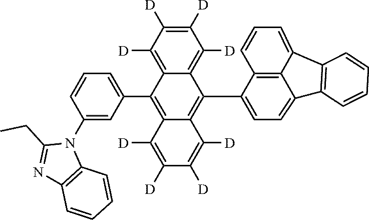 [SMILES: [2H]c1c([2H])c([2H])c2c(-c3ccc4c5c(cccc35)-c3ccccc3-4)c3c([2H])c([2H])c([2H])c([2H])c3c(-c3cccc(-n4c(CC)nc5ccccc54)c3)c2c1[2H]]

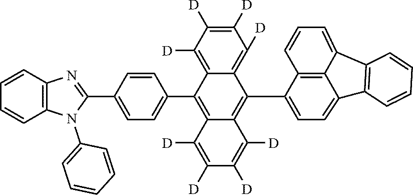 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4nc5ccccc5n4-c4ccccc4)cc3)c2c1[2H]]

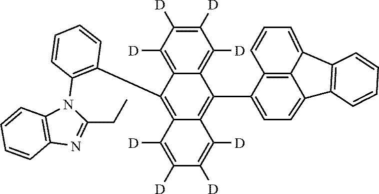 [SMILES: [2H]c1c([2H])c([2H])c2c(-c3ccc4c5c(cccc35)-c3ccccc3-4)c3c([2H])c([2H])c([2H])c([2H])c3c(-c3ccccc3-n3c(CC)nc4ccccc43)c2c1[2H]]

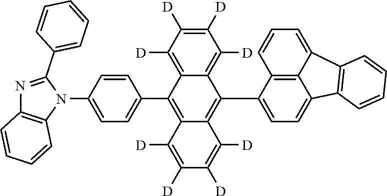 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(-c5ccccc5)nc5ccccc54)cc3)c2c1[2H]]

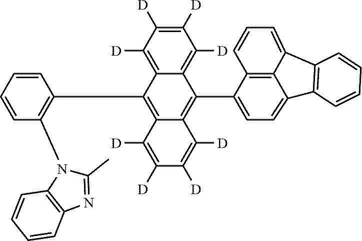 [SMILES: [2H]c1c([2H])c([2H])c2c(-c3ccc4c5c(cccc35)-c3ccccc3-4)c3c([2H])c([2H])c([2H])c([2H])c3c(-c3ccccc3-n3c(C)nc4ccccc43)c2c1[2H]]

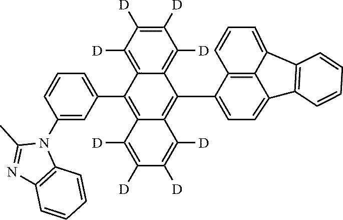 [SMILES: [2H]c1c([2H])c([2H])c2c(-c3ccc4c5c(cccc35)-c3ccccc3-4)c3c([2H])c([2H])c([2H])c([2H])c3c(-c3cccc(-n4c(C)nc5ccccc54)c3)c2c1[2H]]

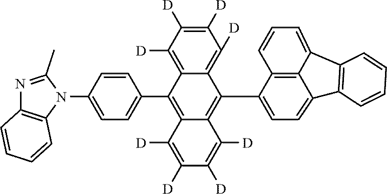 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(C)nc5ccccc54)cc3)c2c1[2H]]

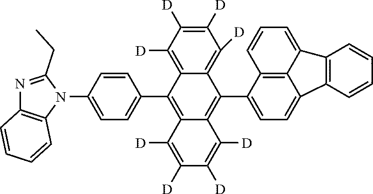 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(CC)nc5ccccc54)cc3)c2c1[2H]]

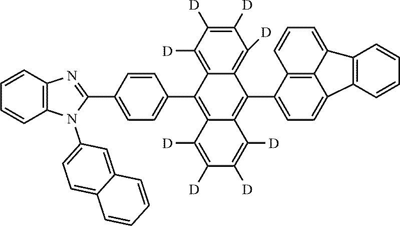 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4nc5ccccc5n4-c4ccc5ccccc5c4)cc3)c2c1[2H]]

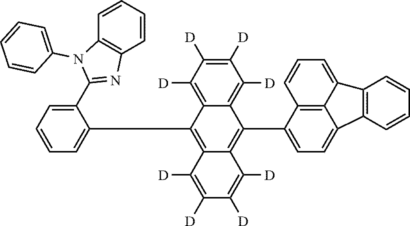 [SMILES: [2H]c1c([2H])c([2H])c2c(-c3ccc4c5c(cccc35)-c3ccccc3-4)c3c([2H])c([2H])c([2H])c([2H])c3c(-c3ccccc3-c3nc4ccccc4n3-c3ccccc3)c2c1[2H]]

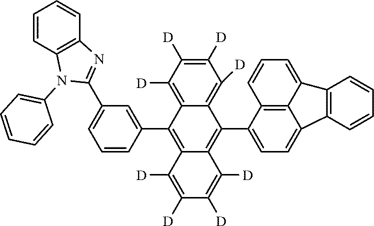 [SMILES: [2H]c1c([2H])c([2H])c2c(-c3ccc4c5c(cccc35)-c3ccccc3-4)c3c([2H])c([2H])c([2H])c([2H])c3c(-c3cccc(-c4nc5ccccc5n4-c4ccccc4)c3)c2c1[2H]]

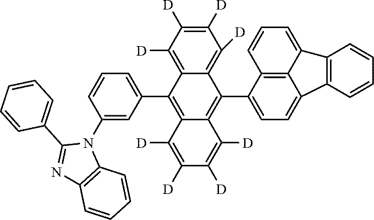 [SMILES: [2H]c1c([2H])c([2H])c2c(-c3ccc4c5c(cccc35)-c3ccccc3-4)c3c([2H])c([2H])c([2H])c([2H])c3c(-c3cccc(-n4c(-c5ccccc5)nc5ccccc54)c3)c2c1[2H]]

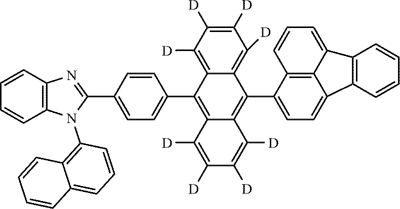 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4nc5ccccc5n4-c4cccc5ccccc45)cc3)c2c1[2H]]

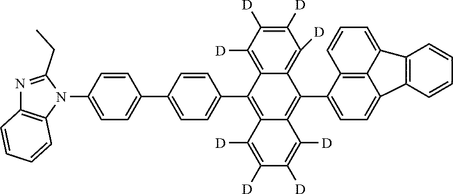 [SMILES: [2H]c1c([2H])c([2H])c2c(-c3ccc4c5c(cccc35)-c3ccccc3-4)c3c([2H])c([2H])c([2H])c([2H])c3c(-c3ccc(-c4ccc(-n5c(CC)nc6ccccc65)cc4)cc3)c2c1[2H]]

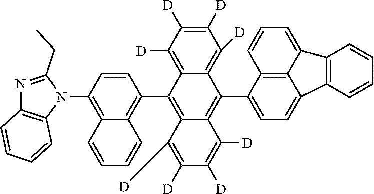 [SMILES: [2H]c1c([2H])c([2H])c2c(-c3ccc4c5c(cccc35)-c3ccccc3-4)c3c([2H])c([2H])c([2H])c([2H])c3c(-c3ccc(-n4c(CC)nc5ccccc54)c4ccccc34)c2c1[2H]]

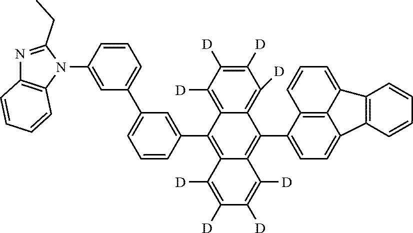 [SMILES: [2H]c1c([2H])c([2H])c2c(-c3ccc4c5c(cccc35)-c3ccccc3-4)c3c([2H])c([2H])c([2H])c([2H])c3c(-c3cccc(-c4cccc(-n5c(CC)nc6ccccc65)c4)c3)c2c1[2H]]

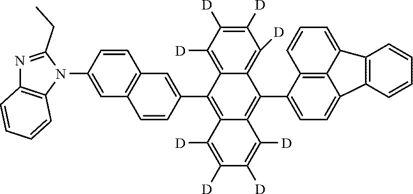 [SMILES: [2H]c1c([2H])c([2H])c2c(-c3ccc4c5c(cccc35)-c3ccccc3-4)c3c([2H])c([2H])c([2H])c([2H])c3c(-c3ccc4cc(-n5c(CC)nc6ccccc65)ccc4c3)c2c1[2H]]

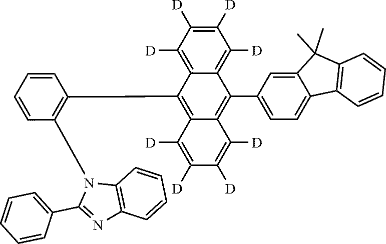 [SMILES: [2H]c1c([2H])c([2H])c2c(-c3ccccc3-n3c(-c4ccccc4)nc4ccccc43)c3c([2H])c([2H])c([2H])c([2H])c3c(-c3ccc4c(c3)C(C)(C)c3ccccc3-4)c2c1[2H]]

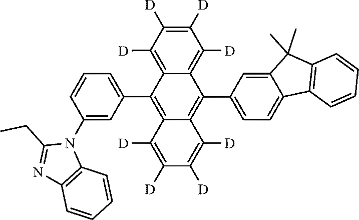 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-n4c(CC)nc5ccccc54)c3)c2c1[2H]]

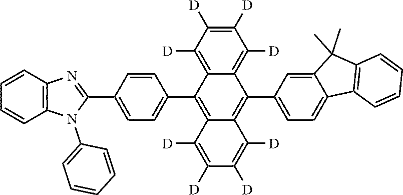 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4nc5ccccc5n4-c4ccccc4)cc3)c2c1[2H]]

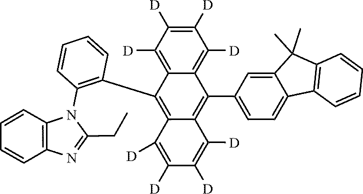 [SMILES: [2H]c1c([2H])c([2H])c2c(-c3ccccc3-n3c(CC)nc4ccccc43)c3c([2H])c([2H])c([2H])c([2H])c3c(-c3ccc4c(c3)C(C)(C)c3ccccc3-4)c2c1[2H]]

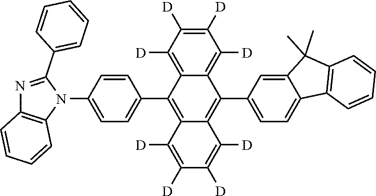 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-n4c(-c5ccccc5)nc5ccccc54)cc3)c2c1[2H]]

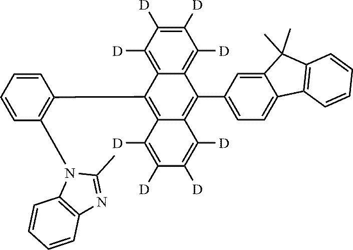 [SMILES: [2H]c1c([2H])c([2H])c2c(-c3ccccc3-n3c(C)nc4ccccc43)c3c([2H])c([2H])c([2H])c([2H])c3c(-c3ccc4c(c3)C(C)(C)c3ccccc3-4)c2c1[2H]]

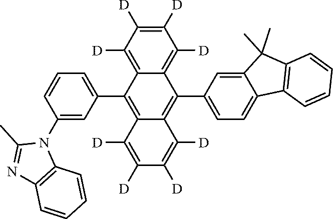 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-n4c(C)nc5ccccc54)c3)c2c1[2H]]

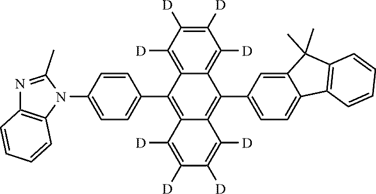 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-n4c(C)nc5ccccc54)cc3)c2c1[2H]]

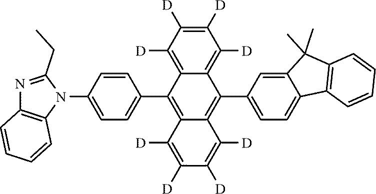 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-n4c(CC)nc5ccccc54)cc3)c2c1[2H]]

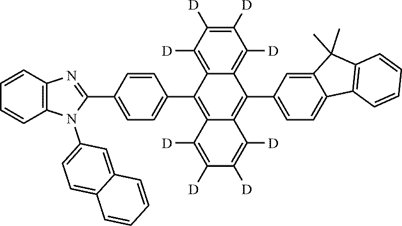 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4nc5ccccc5n4-c4ccc5ccccc5c4)cc3)c2c1[2H]]

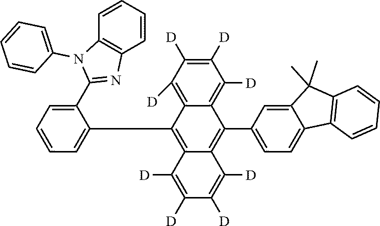 [SMILES: [2H]c1c([2H])c([2H])c2c(-c3ccccc3-c3nc4ccccc4n3-c3ccccc3)c3c([2H])c([2H])c([2H])c([2H])c3c(-c3ccc4c(c3)C(C)(C)c3ccccc3-4)c2c1[2H]]

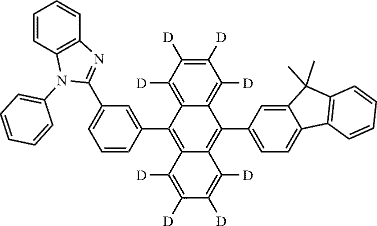 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-c4nc5ccccc5n4-c4ccccc4)c3)c2c1[2H]]

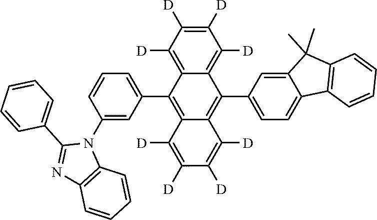 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-n4c(-c5ccccc5)nc5ccccc54)c3)c2c1[2H]]

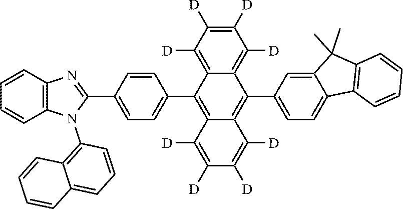 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4nc5ccccc5n4-c4cccc5ccccc45)cc3)c2c1[2H]]

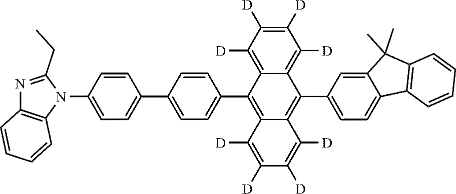 [SMILES: [2H]c1c([2H])c([2H])c2c(-c3ccc4c(c3)C(C)(C)c3ccccc3-4)c3c([2H])c([2H])c([2H])c([2H])c3c(-c3ccc(-c4ccc(-n5c(CC)nc6ccccc65)cc4)cc3)c2c1[2H]]

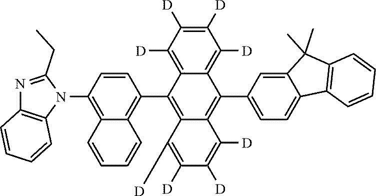 [SMILES: [2H]c1c([2H])c([2H])c2c(-c3ccc(-n4c(CC)nc5ccccc54)c4ccccc34)c3c([2H])c([2H])c([2H])c([2H])c3c(-c3ccc4c(c3)C(C)(C)c3ccccc3-4)c2c1[2H]]

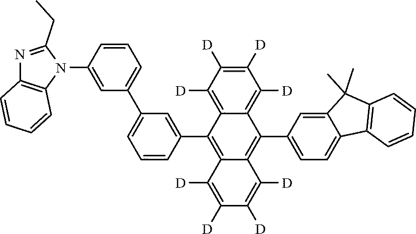 [SMILES: [2H]c1c([2H])c([2H])c2c(-c3ccc4c(c3)C(C)(C)c3ccccc3-4)c3c([2H])c([2H])c([2H])c([2H])c3c(-c3cccc(-c4cccc(-n5c(CC)nc6ccccc65)c4)c3)c2c1[2H]]

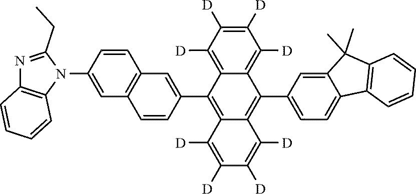 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3ccc4c(c3)C(C)(C)c3ccccc3-4)c2c1[2H]]

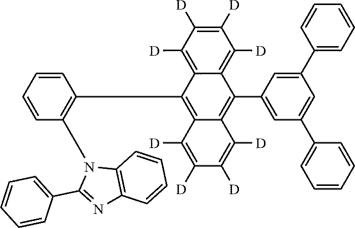 [SMILES: [2H]c1c([2H])c([2H])c2c(-c3ccccc3-n3c(-c4ccccc4)nc4ccccc43)c3c([2H])c([2H])c([2H])c([2H])c3c(-c3cc(-c4ccccc4)cc(-c4ccccc4)c3)c2c1[2H]]

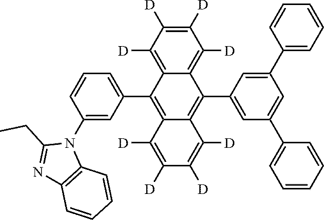 [SMILES: [2H]c1c([2H])c([2H])c2c(-c3cccc(-n4c(CC)nc5ccccc54)c3)c3c([2H])c([2H])c([2H])c([2H])c3c(-c3cc(-c4ccccc4)cc(-c4ccccc4)c3)c2c1[2H]]

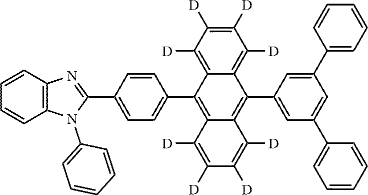 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4nc5ccccc5n4-c4ccccc4)cc3)c2c1[2H]]

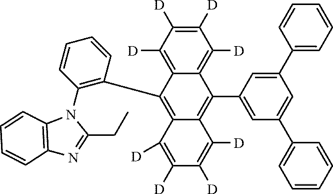 [SMILES: [2H]c1c([2H])c([2H])c2c(-c3ccccc3-n3c(CC)nc4ccccc43)c3c([2H])c([2H])c([2H])c([2H])c3c(-c3cc(-c4ccccc4)cc(-c4ccccc4)c3)c2c1[2H]]

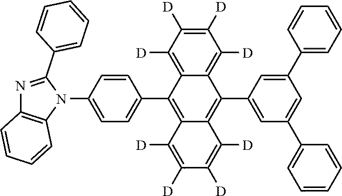 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-n4c(-c5ccccc5)nc5ccccc54)cc3)c2c1[2H]]

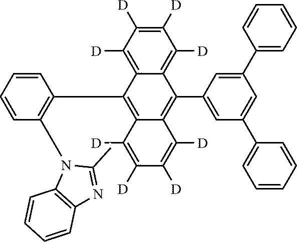 [SMILES: [2H]c1c([2H])c([2H])c2c(-c3ccccc3-n3c(C)nc4ccccc43)c3c([2H])c([2H])c([2H])c([2H])c3c(-c3cc(-c4ccccc4)cc(-c4ccccc4)c3)c2c1[2H]]

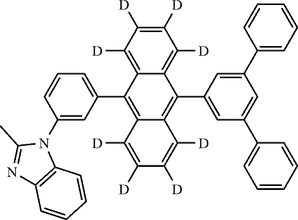 [SMILES: [2H]c1c([2H])c([2H])c2c(-c3cccc(-n4c(C)nc5ccccc54)c3)c3c([2H])c([2H])c([2H])c([2H])c3c(-c3cc(-c4ccccc4)cc(-c4ccccc4)c3)c2c1[2H]]

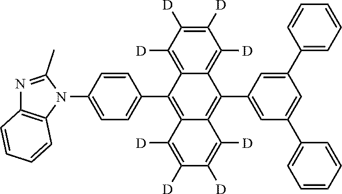 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-n4c(C)nc5ccccc54)cc3)c2c1[2H]]

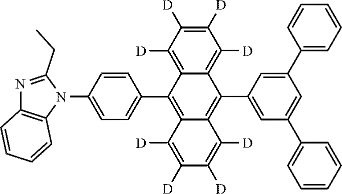 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-n4c(CC)nc5ccccc54)cc3)c2c1[2H]]

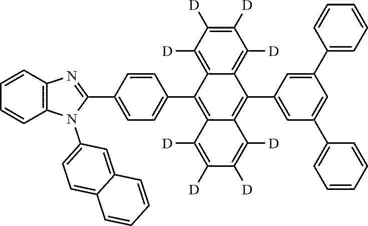 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4nc5ccccc5n4-c4ccc5ccccc5c4)cc3)c2c1[2H]]

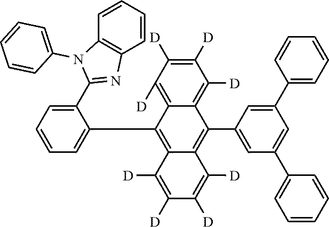 [SMILES: [2H]c1c([2H])c([2H])c2c(-c3ccccc3-c3nc4ccccc4n3-c3ccccc3)c3c([2H])c([2H])c([2H])c([2H])c3c(-c3cc(-c4ccccc4)cc(-c4ccccc4)c3)c2c1[2H]]

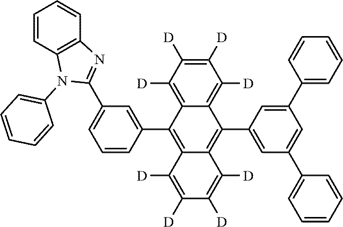 [SMILES: [2H]c1c([2H])c([2H])c2c(-c3cccc(-c4nc5ccccc5n4-c4ccccc4)c3)c3c([2H])c([2H])c([2H])c([2H])c3c(-c3cc(-c4ccccc4)cc(-c4ccccc4)c3)c2c1[2H]]

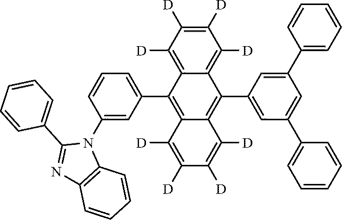 [SMILES: [2H]c1c([2H])c([2H])c2c(-c3cccc(-n4c(-c5ccccc5)nc5ccccc54)c3)c3c([2H])c([2H])c([2H])c([2H])c3c(-c3cc(-c4ccccc4)cc(-c4ccccc4)c3)c2c1[2H]]

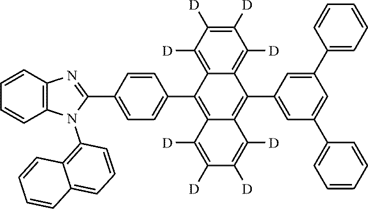 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4nc5ccccc5n4-c4cccc5ccccc45)cc3)c2c1[2H]]

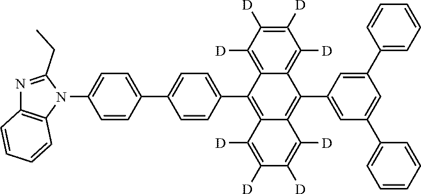 [SMILES: [2H]c1c([2H])c([2H])c2c(-c3cc(-c4ccccc4)cc(-c4ccccc4)c3)c3c([2H])c([2H])c([2H])c([2H])c3c(-c3ccc(-c4ccc(-n5c(CC)nc6ccccc65)cc4)cc3)c2c1[2H]]

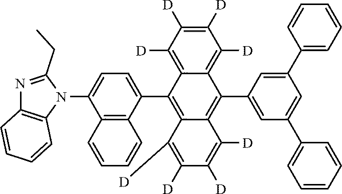 [SMILES: [2H]c1c([2H])c([2H])c2c(-c3ccc(-n4c(CC)nc5ccccc54)c4ccccc34)c3c([2H])c([2H])c([2H])c([2H])c3c(-c3cc(-c4ccccc4)cc(-c4ccccc4)c3)c2c1[2H]]

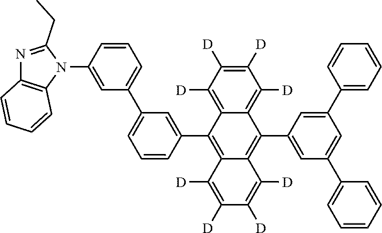 [SMILES: [2H]c1c([2H])c([2H])c2c(-c3cc(-c4ccccc4)cc(-c4ccccc4)c3)c3c([2H])c([2H])c([2H])c([2H])c3c(-c3cccc(-c4cccc(-n5c(CC)nc6ccccc65)c4)c3)c2c1[2H]]

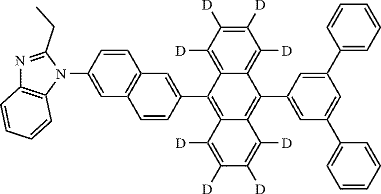 [SMILES: [2H]c1c([2H])c([2H])c2c(-c3ccc4cc(-n5c(CC)nc6ccccc65)ccc4c3)c3c([2H])c([2H])c([2H])c([2H])c3c(-c3cc(-c4ccccc4)cc(-c4ccccc4)c3)c2c1[2H]]

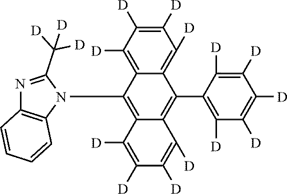 [SMILES: [2H]c1c([2H])c([2H])c(-c2c3c([2H])c([2H])c([2H])c([2H])c3c(-n3c(C([2H])([2H])[2H])nc4ccccc43)c3c([2H])c([2H])c([2H])c([2H])c23)c([2H])c1[2H]]

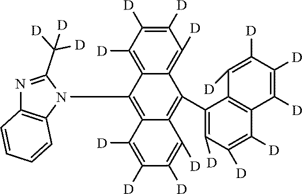 [SMILES: [2H]c1c([2H])c([2H])c2c(-c3c4c([2H])c([2H])c([2H])c([2H])c4c(-n4c(C([2H])([2H])[2H])nc5ccccc54)c4c([2H])c([2H])c([2H])c([2H])c34)c([2H])c([2H])c([2H])c2c1[2H]]

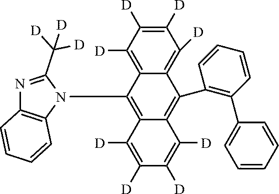 [SMILES: [2H]c1c([2H])c([2H])c2c(-n3c(C([2H])([2H])[2H])nc4ccccc43)c3c([2H])c([2H])c([2H])c([2H])c3c(-c3ccccc3-c3ccccc3)c2c1[2H]]

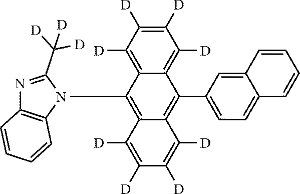 [SMILES: [2H]c1c([2H])c([2H])c2c(-n3c(C([2H])([2H])[2H])nc4ccccc43)c3c([2H])c([2H])c([2H])c([2H])c3c(-c3ccc4ccccc4c3)c2c1[2H]]

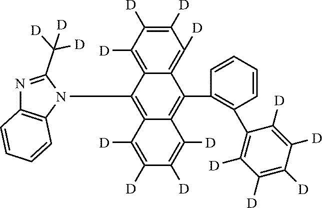 [SMILES: [2H]c1c([2H])c([2H])c(-c2ccccc2-c2c3c([2H])c([2H])c([2H])c([2H])c3c(-n3c(C([2H])([2H])[2H])nc4ccccc43)c3c([2H])c([2H])c([2H])c([2H])c23)c([2H])c1[2H]]

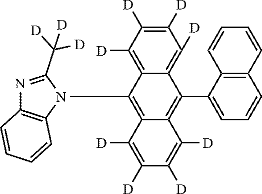 [SMILES: [2H]c1c([2H])c([2H])c2c(-n3c(C([2H])([2H])[2H])nc4ccccc43)c3c([2H])c([2H])c([2H])c([2H])c3c(-c3cccc4ccccc34)c2c1[2H]]

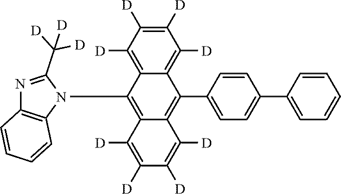 [SMILES: [2H]c1c([2H])c([2H])c2c(-n3c(C([2H])([2H])[2H])nc4ccccc43)c3c([2H])c([2H])c([2H])c([2H])c3c(-c3ccc(-c4ccccc4)cc3)c2c1[2H]]

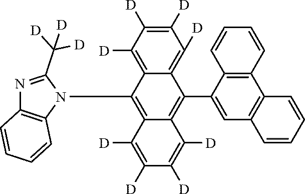 [SMILES: [2H]c1c([2H])c([2H])c2c(-n3c(C([2H])([2H])[2H])nc4ccccc43)c3c([2H])c([2H])c([2H])c([2H])c3c(-c3cc4ccccc4c4ccccc34)c2c1[2H]]

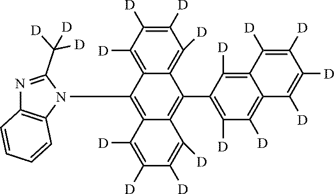 [SMILES: [2H]c1c([2H])c([2H])c2c([2H])c(-c3c4c([2H])c([2H])c([2H])c([2H])c4c(-n4c(C([2H])([2H])[2H])nc5ccccc54)c4c([2H])c([2H])c([2H])c([2H])c34)c([2H])c([2H])c2c1[2H]]

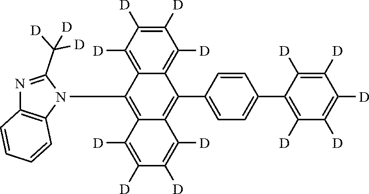 [SMILES: [2H]c1c([2H])c([2H])c(-c2ccc(-c3c4c([2H])c([2H])c([2H])c([2H])c4c(-n4c(C([2H])([2H])[2H])nc5ccccc54)c4c([2H])c([2H])c([2H])c([2H])c34)cc2)c([2H])c1[2H]]

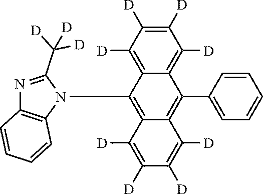 [SMILES: [2H]c1c([2H])c([2H])c2c(-n3c(C([2H])([2H])[2H])nc4ccccc43)c3c([2H])c([2H])c([2H])c([2H])c3c(-c3ccccc3)c2c1[2H]]

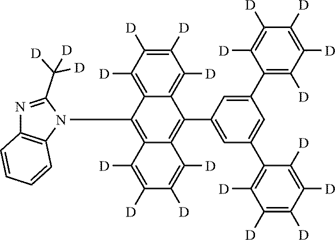 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(C([2H])([2H])[2H])nc5ccccc54)c4c([2H])c([2H])c([2H])c([2H])c34)c2)c([2H])c1[2H]]

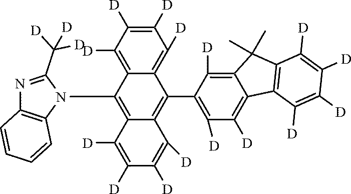 [SMILES: [2H]c1c([2H])c([2H])c2c(c1[2H])-c1c([2H])c([2H])c(-c3c4c([2H])c([2H])c([2H])c([2H])c4c(-n4c(C([2H])([2H])[2H])nc5ccccc54)c4c([2H])c([2H])c([2H])c([2H])c34)c([2H])c1C2(C)C]

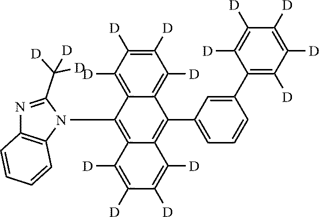 [SMILES: [2H]c1c([2H])c([2H])c(-c2cccc(-c3c4c([2H])c([2H])c([2H])c([2H])c4c(-n4c(C([2H])([2H])[2H])nc5ccccc54)c4c([2H])c([2H])c([2H])c([2H])c34)c2)c([2H])c1[2H]]

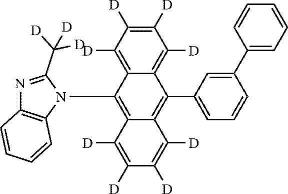 [SMILES: [2H]c1c([2H])c([2H])c2c(-n3c(C([2H])([2H])[2H])nc4ccccc43)c3c([2H])c([2H])c([2H])c([2H])c3c(-c3cccc(-c4ccccc4)c3)c2c1[2H]]

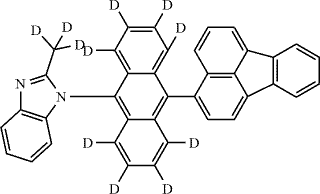 [SMILES: [2H]c1c([2H])c([2H])c2c(-n3c(C([2H])([2H])[2H])nc4ccccc43)c3c([2H])c([2H])c([2H])c([2H])c3c(-c3ccc4c5c(cccc35)-c3ccccc3-4)c2c1[2H]]

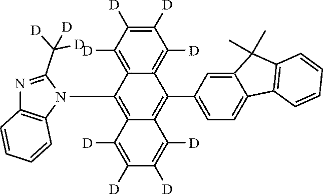 [SMILES: [2H]c1c([2H])c([2H])c2c(-n3c(C([2H])([2H])[2H])nc4ccccc43)c3c([2H])c([2H])c([2H])c([2H])c3c(-c3ccc4c(c3)C(C)(C)c3ccccc3-4)c2c1[2H]]

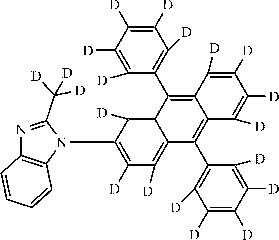 [SMILES: [2H]C1=C2C(c3c([2H])c([2H])c([2H])c([2H])c3[2H])=c3c([2H])c([2H])c([2H])c([2H])c3=C(c3c([2H])c([2H])c([2H])c([2H])c3[2H])C2C([2H])C(n2c(C([2H])([2H])[2H])nc3ccccc32)=C1[2H]]

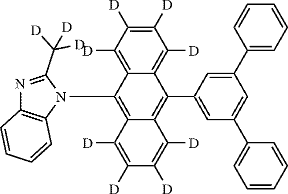 [SMILES: [2H]c1c([2H])c([2H])c2c(-n3c(C([2H])([2H])[2H])nc4ccccc43)c3c([2H])c([2H])c([2H])c([2H])c3c(-c3cc(-c4ccccc4)cc(-c4ccccc4)c3)c2c1[2H]]

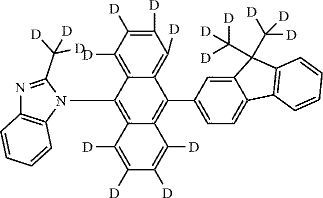 [SMILES: [2H]c1c([2H])c([2H])c2c(-n3c(C([2H])([2H])[2H])nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

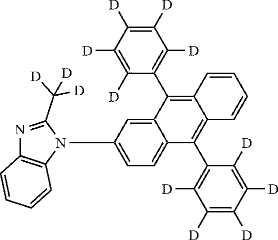 [SMILES: [2H]c1c([2H])c([2H])c(-c2c3ccccc3c(-c3c([2H])c([2H])c([2H])c([2H])c3[2H])c3cc(-n4c(C([2H])([2H])[2H])nc5ccccc54)ccc23)c([2H])c1[2H]]

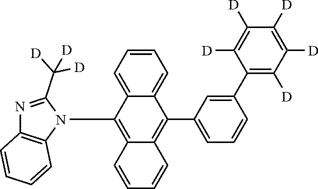 [SMILES: [2H]c1c([2H])c([2H])c(-c2cccc(-c3c4ccccc4c(-n4c(C([2H])([2H])[2H])nc5ccccc54)c4ccccc34)c2)c([2H])c1[2H]]

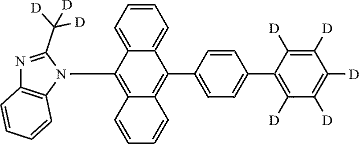 [SMILES: [2H]c1c([2H])c([2H])c(-c2ccc(-c3c4ccccc4c(-n4c(C([2H])([2H])[2H])nc5ccccc54)c4ccccc34)cc2)c([2H])c1[2H]]

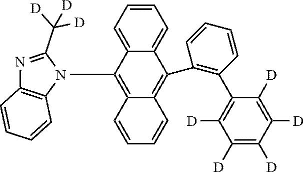 [SMILES: [2H]c1c([2H])c([2H])c(-c2ccccc2-c2c3ccccc3c(-n3c(C([2H])([2H])[2H])nc4ccccc43)c3ccccc23)c([2H])c1[2H]]

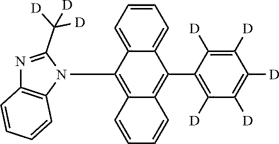 [SMILES: [2H]c1c([2H])c([2H])c(-c2c3ccccc3c(-n3c(C([2H])([2H])[2H])nc4ccccc43)c3ccccc23)c([2H])c1[2H]]

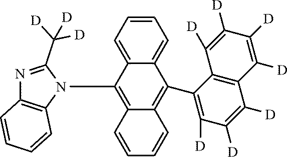 [SMILES: [2H]c1c([2H])c([2H])c2c(-c3c4ccccc4c(-n4c(C([2H])([2H])[2H])nc5ccccc54)c4ccccc34)c([2H])c([2H])c([2H])c2c1[2H]]

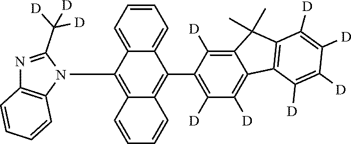 [SMILES: [2H]c1c([2H])c([2H])c2c(c1[2H])-c1c([2H])c([2H])c(-c3c4ccccc4c(-n4c(C([2H])([2H])[2H])nc5ccccc54)c4ccccc34)c([2H])c1C2(C)C]

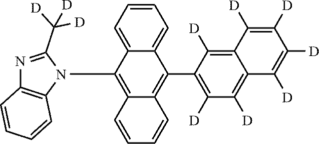 [SMILES: [2H]c1c([2H])c([2H])c2c([2H])c(-c3c4ccccc4c(-n4c(C([2H])([2H])[2H])nc5ccccc54)c4ccccc34)c([2H])c([2H])c2c1[2H]]

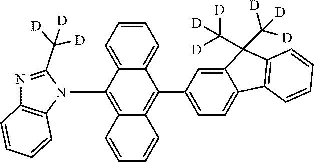 [SMILES: [2H]C([2H])([2H])c1nc2ccccc2n1-c1c2ccccc2c(-c2ccc3c(c2)C(C([2H])([2H])[2H])(C([2H])([2H])[2H])c2ccccc2-3)c2ccccc12]

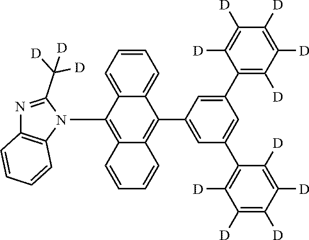 [SMILES: [2H]c1c([2H])c([2H])c(-c2cc(-c3c([2H])c([2H])c([2H])c([2H])c3[2H])cc(-c3c4ccccc4c(-n4c(C([2H])([2H])[2H])nc5ccccc54)c4ccccc34)c2)c([2H])c1[2H]]

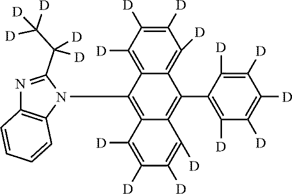 [SMILES: [2H]c1c([2H])c([2H])c(-c2c3c([2H])c([2H])c([2H])c([2H])c3c(-n3c(C([2H])([2H])C([2H])([2H])[2H])nc4ccccc43)c3c([2H])c([2H])c([2H])c([2H])c23)c([2H])c1[2H]]

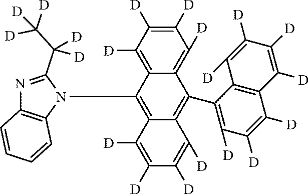 [SMILES: [2H]c1c([2H])c([2H])c2c(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)c([2H])c([2H])c([2H])c2c1[2H]]

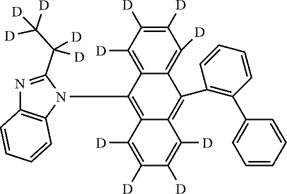 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccccc3-c3ccccc3)c2c1[2H]]

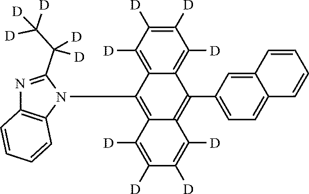 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccc4ccccc4c3)c2c1[2H]]

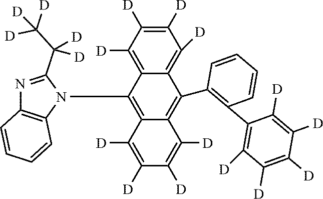 [SMILES: [2H]c1c([2H])c([2H])c(-c2ccccc2-c2c3c([2H])c([2H])c([2H])c([2H])c3c(-n3c(C([2H])([2H])C([2H])([2H])[2H])nc4ccccc43)c3c([2H])c([2H])c([2H])c([2H])c23)c([2H])c1[2H]]

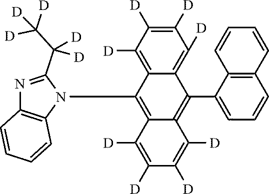 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3cccc4ccccc34)c2c1[2H]]

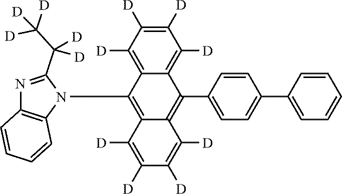 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccc(-c4ccccc4)cc3)c2c1[2H]]

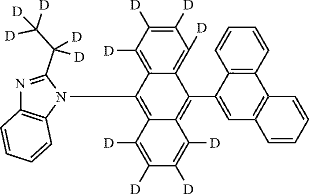 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3cc4ccccc4c4ccccc34)c2c1[2H]]

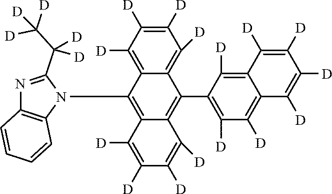 [SMILES: [2H]c1c([2H])c([2H])c2c([2H])c(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)c([2H])c([2H])c2c1[2H]]

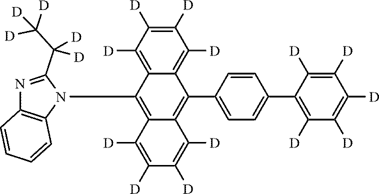 [SMILES: [2H]c1c([2H])c([2H])c(-c2ccc(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)cc2)c([2H])c1[2H]]

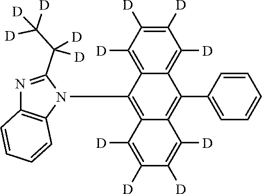 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccccc3)c2c1[2H]]

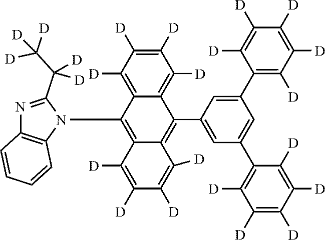 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)c2)c([2H])c1[2H]]

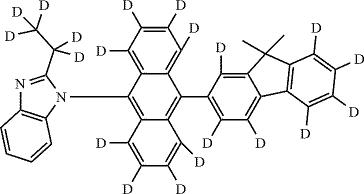 [SMILES: [2H]c1c([2H])c([2H])c2c(c1[2H])-c1c([2H])c([2H])c(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)c([2H])c1C2(C)C]

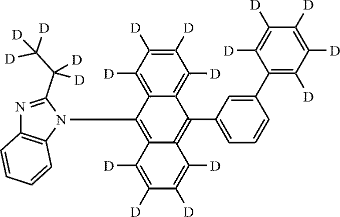 [SMILES: [2H]c1c([2H])c([2H])c(-c2cccc(-c3c4c([2H])c([2H])c([2H])c([2H])c4c(-n4c(C([2H])([2H])C([2H])([2H])[2H])nc5ccccc54)c4c([2H])c([2H])c([2H])c([2H])c34)c2)c([2H])c1[2H]]

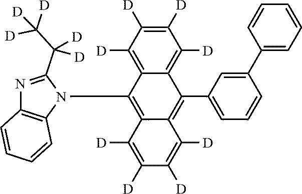 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3cccc(-c4ccccc4)c3)c2c1[2H]]

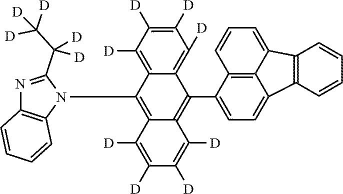 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccc4c5c(cccc35)-c3ccccc3-4)c2c1[2H]]

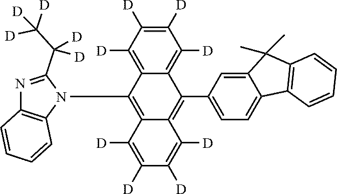 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccc4c(c3)C(C)(C)c3ccccc3-4)c2c1[2H]]

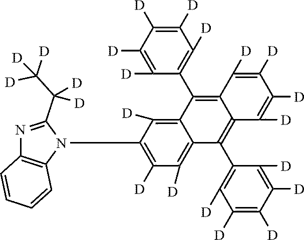 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-n4c(C([2H])([2H])C([2H])([2H])[2H])nc5ccccc54)c([2H])c([2H])c23)c([2H])c1[2H]]

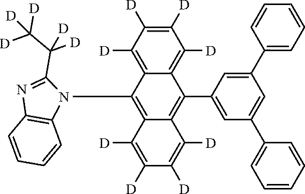 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3cc(-c4ccccc4)cc(-c4ccccc4)c3)c2c1[2H]]

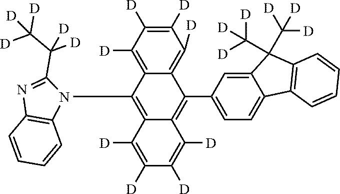 [SMILES: [2H]c1c([2H])c([2H])c2c(-n3c(C([2H])([2H])C([2H])([2H])[2H])nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

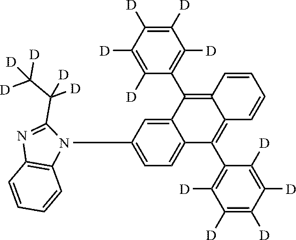 [SMILES: [2H]c1c([2H])c([2H])c(-c2c3ccccc3c(-c3c([2H])c([2H])c([2H])c([2H])c3[2H])c3cc(-n4c(C([2H])([2H])C([2H])([2H])[2H])nc5ccccc54)ccc23)c([2H])c1[2H]]

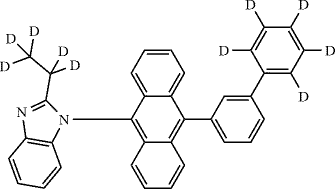 [SMILES: [2H]c1c([2H])c([2H])c(-c2cccc(-c3c4ccccc4c(-n4c(C([2H])([2H])C([2H])([2H])[2H])nc5ccccc54)c4ccccc34)c2)c([2H])c1[2H]]

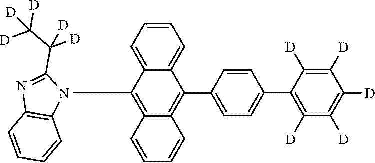 [SMILES: [2H]c1c([2H])c([2H])c(-c2ccc(-c3c4ccccc4c(-n4c(C([2H])([2H])C([2H])([2H])[2H])nc5ccccc54)c4ccccc34)cc2)c([2H])c1[2H]]

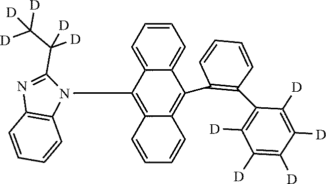 [SMILES: [2H]c1c([2H])c([2H])c(-c2ccccc2-c2c3ccccc3c(-n3c(C([2H])([2H])C([2H])([2H])[2H])nc4ccccc43)c3ccccc23)c([2H])c1[2H]]

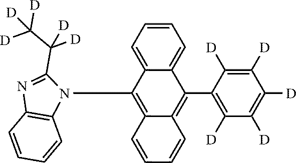 [SMILES: [2H]c1c([2H])c([2H])c(-c2c3ccccc3c(-n3c(C([2H])([2H])C([2H])([2H])[2H])nc4ccccc43)c3ccccc23)c([2H])c1[2H]]

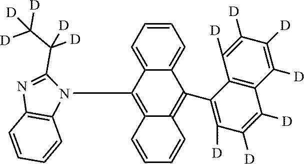 [SMILES: [2H]c1c([2H])c([2H])c2c(-c3c4ccccc4c(-n4c(C([2H])([2H])C([2H])([2H])[2H])nc5ccccc54)c4ccccc34)c([2H])c([2H])c([2H])c2c1[2H]]

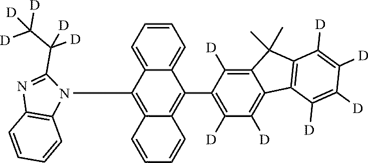 [SMILES: [2H]c1c([2H])c([2H])c2c(c1[2H])-c1c([2H])c([2H])c(-c3c4ccccc4c(-n4c(C([2H])([2H])C([2H])([2H])[2H])nc5ccccc54)c4ccccc34)c([2H])c1C2(C)C]

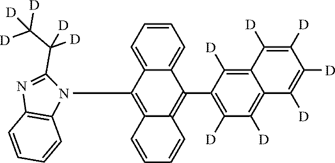 [SMILES: [2H]c1c([2H])c([2H])c2c([2H])c(-c3c4ccccc4c(-n4c(C([2H])([2H])C([2H])([2H])[2H])nc5ccccc54)c4ccccc34)c([2H])c([2H])c2c1[2H]]

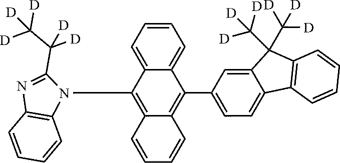 [SMILES: [2H]C([2H])([2H])C([2H])([2H])c1nc2ccccc2n1-c1c2ccccc2c(-c2ccc3c(c2)C(C([2H])([2H])[2H])(C([2H])([2H])[2H])c2ccccc2-3)c2ccccc12]

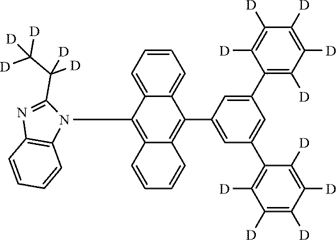 [SMILES: [2H]c1c([2H])c([2H])c(-c2cc(-c3c([2H])c([2H])c([2H])c([2H])c3[2H])cc(-c3c4ccccc4c(-n4c(C([2H])([2H])C([2H])([2H])[2H])nc5ccccc54)c4ccccc34)c2)c([2H])c1[2H]]

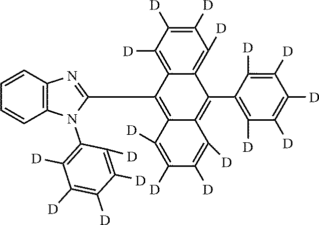 [SMILES: [2H]c1c([2H])c([2H])c(-c2c3c([2H])c([2H])c([2H])c([2H])c3c(-c3nc4ccccc4n3-c3c([2H])c([2H])c([2H])c([2H])c3[2H])c3c([2H])c([2H])c([2H])c([2H])c23)c([2H])c1[2H]]

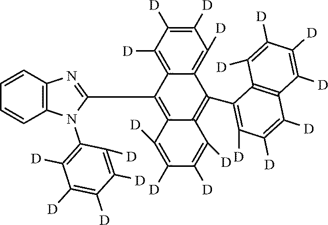 [SMILES: [2H]c1c([2H])c([2H])c(-n2c(-c3c4c([2H])c([2H])c([2H])c([2H])c4c(-c4c([2H])c([2H])c([2H])c5c([2H])c([2H])c([2H])c([2H])c45)c4c([2H])c([2H])c([2H])c([2H])c34)nc3ccccc32)c([2H])c1[2H]]

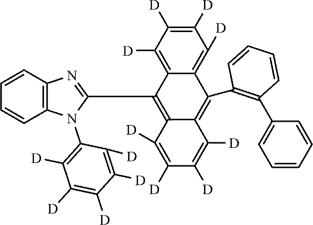 [SMILES: [2H]c1c([2H])c([2H])c(-n2c(-c3c4c([2H])c([2H])c([2H])c([2H])c4c(-c4ccccc4-c4ccccc4)c4c([2H])c([2H])c([2H])c([2H])c34)nc3ccccc32)c([2H])c1[2H]]

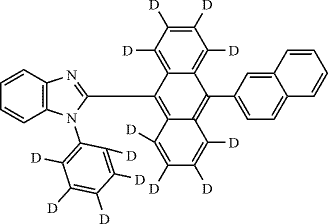 [SMILES: [2H]c1c([2H])c([2H])c(-n2c(-c3c4c([2H])c([2H])c([2H])c([2H])c4c(-c4ccc5ccccc5c4)c4c([2H])c([2H])c([2H])c([2H])c34)nc3ccccc32)c([2H])c1[2H]]

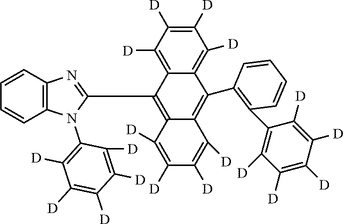 [SMILES: [2H]c1c([2H])c([2H])c(-c2ccccc2-c2c3c([2H])c([2H])c([2H])c([2H])c3c(-c3nc4ccccc4n3-c3c([2H])c([2H])c([2H])c([2H])c3[2H])c3c([2H])c([2H])c([2H])c([2H])c23)c([2H])c1[2H]]

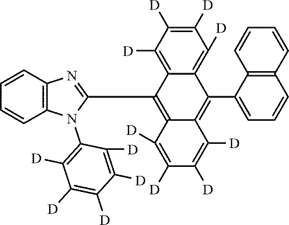 [SMILES: [2H]c1c([2H])c([2H])c(-n2c(-c3c4c([2H])c([2H])c([2H])c([2H])c4c(-c4cccc5ccccc45)c4c([2H])c([2H])c([2H])c([2H])c34)nc3ccccc32)c([2H])c1[2H]]

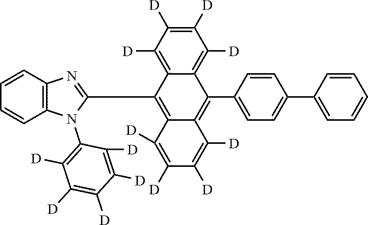 [SMILES: [2H]c1c([2H])c([2H])c(-n2c(-c3c4c([2H])c([2H])c([2H])c([2H])c4c(-c4ccc(-c5ccccc5)cc4)c4c([2H])c([2H])c([2H])c([2H])c34)nc3ccccc32)c([2H])c1[2H]]

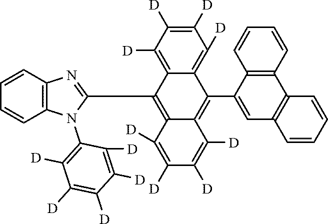 [SMILES: [2H]c1c([2H])c([2H])c(-n2c(-c3c4c([2H])c([2H])c([2H])c([2H])c4c(-c4cc5ccccc5c5ccccc45)c4c([2H])c([2H])c([2H])c([2H])c34)nc3ccccc32)c([2H])c1[2H]]

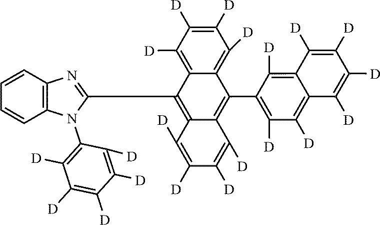 [SMILES: [2H]c1c([2H])c([2H])c(-n2c(-c3c4c([2H])c([2H])c([2H])c([2H])c4c(-c4c([2H])c([2H])c5c([2H])c([2H])c([2H])c([2H])c5c4[2H])c4c([2H])c([2H])c([2H])c([2H])c34)nc3ccccc32)c([2H])c1[2H]]

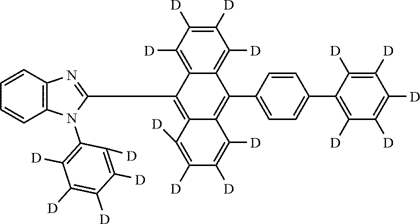 [SMILES: [2H]c1c([2H])c([2H])c(-c2ccc(-c3c4c([2H])c([2H])c([2H])c([2H])c4c(-c4nc5ccccc5n4-c4c([2H])c([2H])c([2H])c([2H])c4[2H])c4c([2H])c([2H])c([2H])c([2H])c34)cc2)c([2H])c1[2H]]

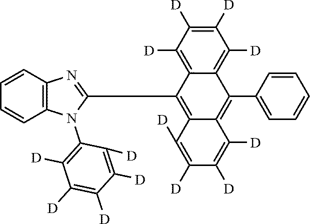 [SMILES: [2H]c1c([2H])c([2H])c(-n2c(-c3c4c([2H])c([2H])c([2H])c([2H])c4c(-c4ccccc4)c4c([2H])c([2H])c([2H])c([2H])c34)nc3ccccc32)c([2H])c1[2H]]

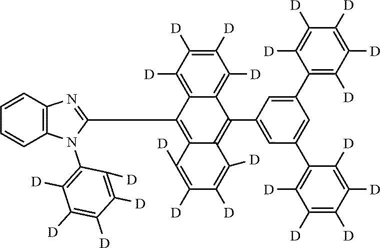 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4nc5ccccc5n4-c4c([2H])c([2H])c([2H])c([2H])c4[2H])c4c([2H])c([2H])c([2H])c([2H])c34)c2)c([2H])c1[2H]]

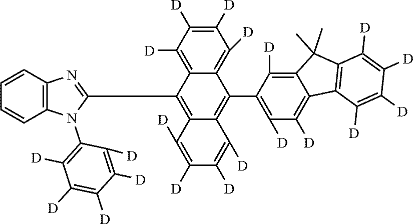 [SMILES: [2H]c1c([2H])c([2H])c(-n2c(-c3c4c([2H])c([2H])c([2H])c([2H])c4c(-c4c([2H])c([2H])c5c(c4[2H])C(C)(C)c4c([2H])c([2H])c([2H])c([2H])c4-5)c4c([2H])c([2H])c([2H])c([2H])c34)nc3ccccc32)c([2H])c1[2H]]

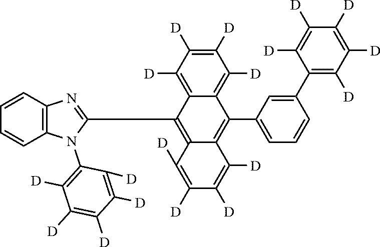 [SMILES: [2H]c1c([2H])c([2H])c(-c2cccc(-c3c4c([2H])c([2H])c([2H])c([2H])c4c(-c4nc5ccccc5n4-c4c([2H])c([2H])c([2H])c([2H])c4[2H])c4c([2H])c([2H])c([2H])c([2H])c34)c2)c([2H])c1[2H]]

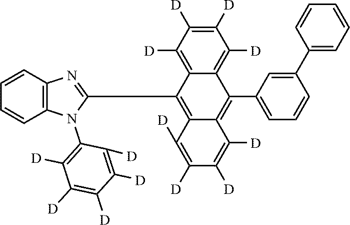 [SMILES: [2H]c1c([2H])c([2H])c(-n2c(-c3c4c([2H])c([2H])c([2H])c([2H])c4c(-c4cccc(-c5ccccc5)c4)c4c([2H])c([2H])c([2H])c([2H])c34)nc3ccccc32)c([2H])c1[2H]]

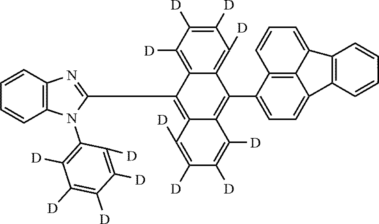 [SMILES: [2H]c1c([2H])c([2H])c(-n2c(-c3c4c([2H])c([2H])c([2H])c([2H])c4c(-c4ccc5c6c(cccc46)-c4ccccc4-5)c4c([2H])c([2H])c([2H])c([2H])c34)nc3ccccc32)c([2H])c1[2H]]

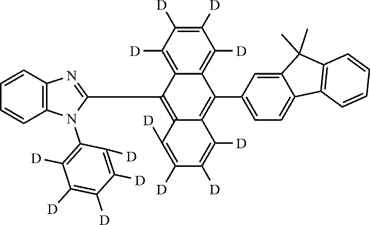 [SMILES: [2H]c1c([2H])c([2H])c(-n2c(-c3c4c([2H])c([2H])c([2H])c([2H])c4c(-c4ccc5c(c4)C(C)(C)c4ccccc4-5)c4c([2H])c([2H])c([2H])c([2H])c34)nc3ccccc32)c([2H])c1[2H]]

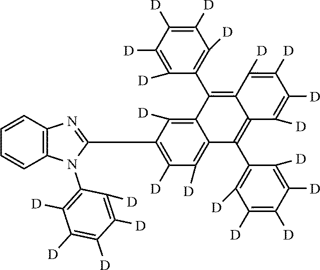 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4nc5ccccc5n4-c4c([2H])c([2H])c([2H])c([2H])c4[2H])c([2H])c([2H])c23)c([2H])c1[2H]]

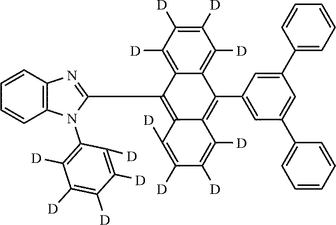 [SMILES: [2H]c1c([2H])c([2H])c(-n2c(-c3c4c([2H])c([2H])c([2H])c([2H])c4c(-c4cc(-c5ccccc5)cc(-c5ccccc5)c4)c4c([2H])c([2H])c([2H])c([2H])c34)nc3ccccc32)c([2H])c1[2H]]

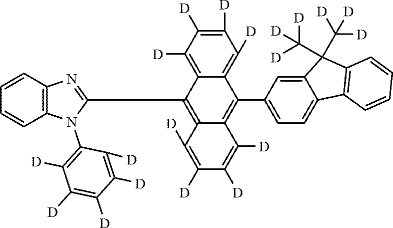 [SMILES: [2H]c1c([2H])c([2H])c(-n2c(-c3c4c([2H])c([2H])c([2H])c([2H])c4c(-c4ccc5c(c4)C(C([2H])([2H])[2H])(C([2H])([2H])[2H])c4ccccc4-5)c4c([2H])c([2H])c([2H])c([2H])c34)nc3ccccc32)c([2H])c1[2H]]

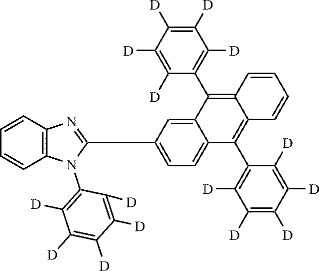 [SMILES: [2H]c1c([2H])c([2H])c(-c2c3ccccc3c(-c3c([2H])c([2H])c([2H])c([2H])c3[2H])c3cc(-c4nc5ccccc5n4-c4c([2H])c([2H])c([2H])c([2H])c4[2H])ccc23)c([2H])c1[2H]]

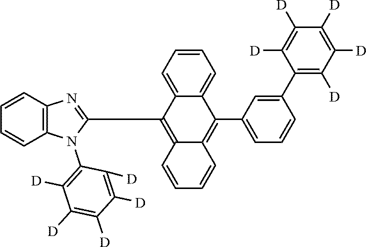 [SMILES: [2H]c1c([2H])c([2H])c(-c2cccc(-c3c4ccccc4c(-c4nc5ccccc5n4-c4c([2H])c([2H])c([2H])c([2H])c4[2H])c4ccccc34)c2)c([2H])c1[2H]]

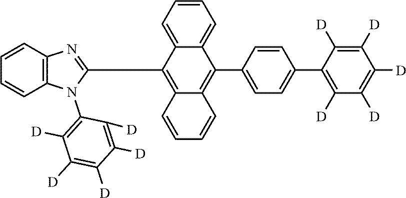 [SMILES: [2H]c1c([2H])c([2H])c(-c2ccc(-c3c4ccccc4c(-c4nc5ccccc5n4-c4c([2H])c([2H])c([2H])c([2H])c4[2H])c4ccccc34)cc2)c([2H])c1[2H]]

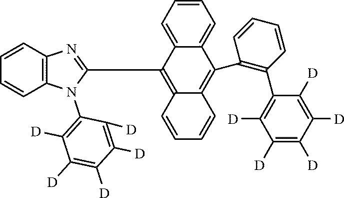 [SMILES: [2H]c1c([2H])c([2H])c(-c2ccccc2-c2c3ccccc3c(-c3nc4ccccc4n3-c3c([2H])c([2H])c([2H])c([2H])c3[2H])c3ccccc23)c([2H])c1[2H]]

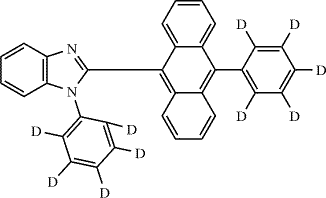 [SMILES: [2H]c1c([2H])c([2H])c(-c2c3ccccc3c(-c3nc4ccccc4n3-c3c([2H])c([2H])c([2H])c([2H])c3[2H])c3ccccc23)c([2H])c1[2H]]

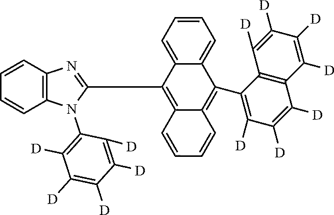 [SMILES: [2H]c1c([2H])c([2H])c(-n2c(-c3c4ccccc4c(-c4c([2H])c([2H])c([2H])c5c([2H])c([2H])c([2H])c([2H])c45)c4ccccc34)nc3ccccc32)c([2H])c1[2H]]

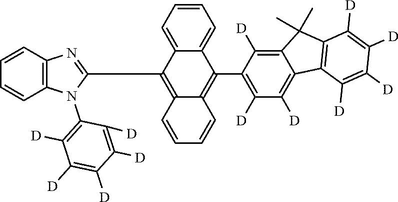 [SMILES: [2H]c1c([2H])c([2H])c(-n2c(-c3c4ccccc4c(-c4c([2H])c([2H])c5c(c4[2H])C(C)(C)c4c([2H])c([2H])c([2H])c([2H])c4-5)c4ccccc34)nc3ccccc32)c([2H])c1[2H]]

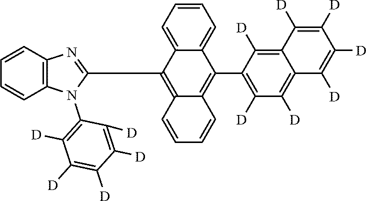 [SMILES: [2H]c1c([2H])c([2H])c(-n2c(-c3c4ccccc4c(-c4c([2H])c([2H])c5c([2H])c([2H])c([2H])c([2H])c5c4[2H])c4ccccc34)nc3ccccc32)c([2H])c1[2H]]

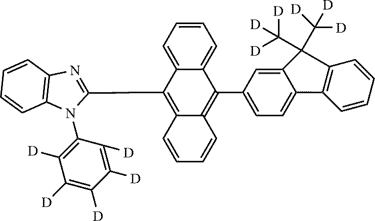 [SMILES: [2H]c1c([2H])c([2H])c(-n2c(-c3c4ccccc4c(-c4ccc5c(c4)C(C([2H])([2H])[2H])(C([2H])([2H])[2H])c4ccccc4-5)c4ccccc34)nc3ccccc32)c([2H])c1[2H]]

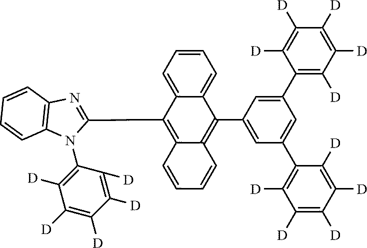 [SMILES: [2H]c1c([2H])c([2H])c(-c2cc(-c3c([2H])c([2H])c([2H])c([2H])c3[2H])cc(-c3c4ccccc4c(-c4nc5ccccc5n4-c4c([2H])c([2H])c([2H])c([2H])c4[2H])c4ccccc34)c2)c([2H])c1[2H]]

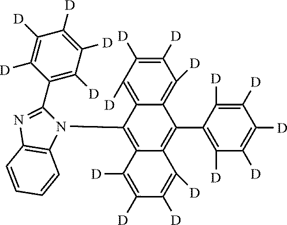 [SMILES: [2H]c1c([2H])c([2H])c(-c2c3c([2H])c([2H])c([2H])c([2H])c3c(-n3c(-c4c([2H])c([2H])c([2H])c([2H])c4[2H])nc4ccccc43)c3c([2H])c([2H])c([2H])c([2H])c23)c([2H])c1[2H]]

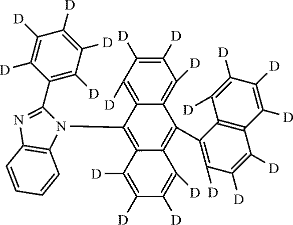 [SMILES: [2H]c1c([2H])c([2H])c(-c2nc3ccccc3n2-c2c3c([2H])c([2H])c([2H])c([2H])c3c(-c3c([2H])c([2H])c([2H])c4c([2H])c([2H])c([2H])c([2H])c34)c3c([2H])c([2H])c([2H])c([2H])c23)c([2H])c1[2H]]

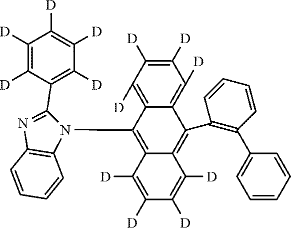 [SMILES: [2H]c1c([2H])c([2H])c(-c2nc3ccccc3n2-c2c3c([2H])c([2H])c([2H])c([2H])c3c(-c3ccccc3-c3ccccc3)c3c([2H])c([2H])c([2H])c([2H])c23)c([2H])c1[2H]]

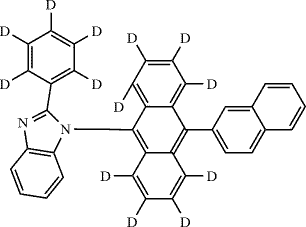 [SMILES: [2H]c1c([2H])c([2H])c(-c2nc3ccccc3n2-c2c3c([2H])c([2H])c([2H])c([2H])c3c(-c3ccc4ccccc4c3)c3c([2H])c([2H])c([2H])c([2H])c23)c([2H])c1[2H]]

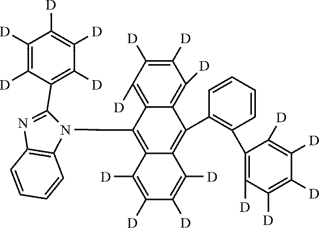 [SMILES: [2H]c1c([2H])c([2H])c(-c2ccccc2-c2c3c([2H])c([2H])c([2H])c([2H])c3c(-n3c(-c4c([2H])c([2H])c([2H])c([2H])c4[2H])nc4ccccc43)c3c([2H])c([2H])c([2H])c([2H])c23)c([2H])c1[2H]]

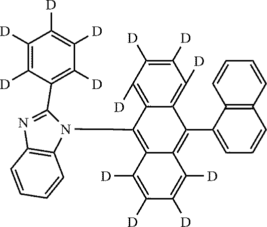 [SMILES: [2H]c1c([2H])c([2H])c(-c2nc3ccccc3n2-c2c3c([2H])c([2H])c([2H])c([2H])c3c(-c3cccc4ccccc34)c3c([2H])c([2H])c([2H])c([2H])c23)c([2H])c1[2H]]

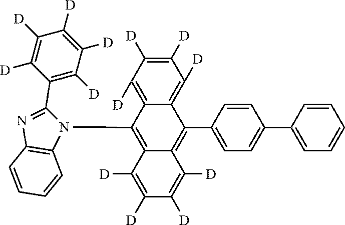 [SMILES: [2H]c1c([2H])c([2H])c(-c2nc3ccccc3n2-c2c3c([2H])c([2H])c([2H])c([2H])c3c(-c3ccc(-c4ccccc4)cc3)c3c([2H])c([2H])c([2H])c([2H])c23)c([2H])c1[2H]]

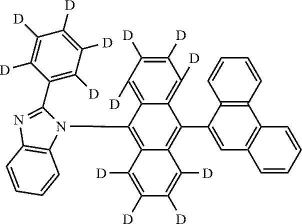 [SMILES: [2H]c1c([2H])c([2H])c(-c2nc3ccccc3n2-c2c3c([2H])c([2H])c([2H])c([2H])c3c(-c3cc4ccccc4c4ccccc34)c3c([2H])c([2H])c([2H])c([2H])c23)c([2H])c1[2H]]

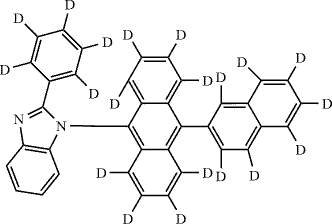 [SMILES: [2H]c1c([2H])c([2H])c(-c2nc3ccccc3n2-c2c3c([2H])c([2H])c([2H])c([2H])c3c(-c3c([2H])c([2H])c4c([2H])c([2H])c([2H])c([2H])c4c3[2H])c3c([2H])c([2H])c([2H])c([2H])c23)c([2H])c1[2H]]

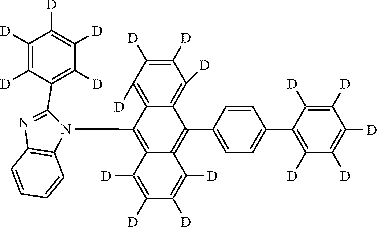 [SMILES: [2H]c1c([2H])c([2H])c(-c2ccc(-c3c4c([2H])c([2H])c([2H])c([2H])c4c(-n4c(-c5c([2H])c([2H])c([2H])c([2H])c5[2H])nc5ccccc54)c4c([2H])c([2H])c([2H])c([2H])c34)cc2)c([2H])c1[2H]]

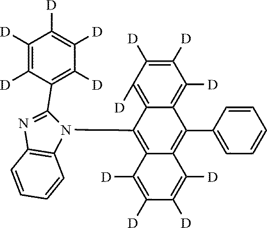 [SMILES: [2H]c1c([2H])c([2H])c(-c2nc3ccccc3n2-c2c3c([2H])c([2H])c([2H])c([2H])c3c(-c3ccccc3)c3c([2H])c([2H])c([2H])c([2H])c23)c([2H])c1[2H]]

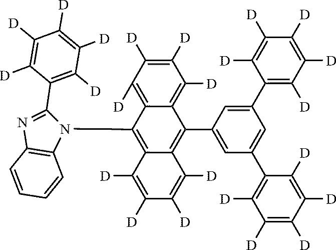 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(-c5c([2H])c([2H])c([2H])c([2H])c5[2H])nc5ccccc54)c4c([2H])c([2H])c([2H])c([2H])c34)c2)c([2H])c1[2H]]

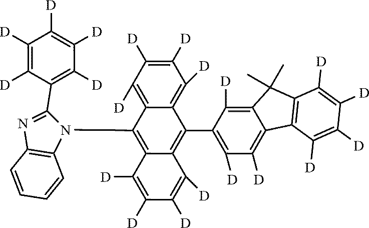 [SMILES: [2H]c1c([2H])c([2H])c(-c2nc3ccccc3n2-c2c3c([2H])c([2H])c([2H])c([2H])c3c(-c3c([2H])c([2H])c4c(c3[2H])C(C)(C)c3c([2H])c([2H])c([2H])c([2H])c3-4)c3c([2H])c([2H])c([2H])c([2H])c23)c([2H])c1[2H]]

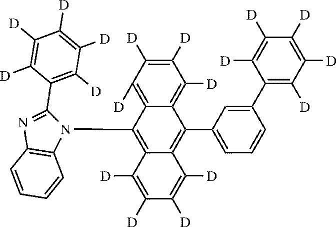 [SMILES: [2H]c1c([2H])c([2H])c(-c2cccc(-c3c4c([2H])c([2H])c([2H])c([2H])c4c(-n4c(-c5c([2H])c([2H])c([2H])c([2H])c5[2H])nc5ccccc54)c4c([2H])c([2H])c([2H])c([2H])c34)c2)c([2H])c1[2H]]

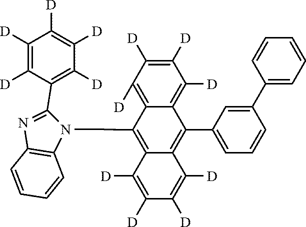 [SMILES: [2H]c1c([2H])c([2H])c(-c2nc3ccccc3n2-c2c3c([2H])c([2H])c([2H])c([2H])c3c(-c3cccc(-c4ccccc4)c3)c3c([2H])c([2H])c([2H])c([2H])c23)c([2H])c1[2H]]

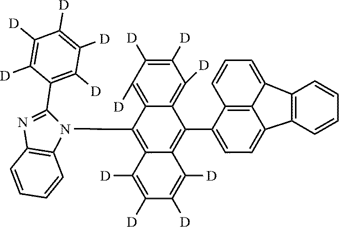 [SMILES: [2H]c1c([2H])c([2H])c(-c2nc3ccccc3n2-c2c3c([2H])c([2H])c([2H])c([2H])c3c(-c3ccc4c5c(cccc35)-c3ccccc3-4)c3c([2H])c([2H])c([2H])c([2H])c23)c([2H])c1[2H]]

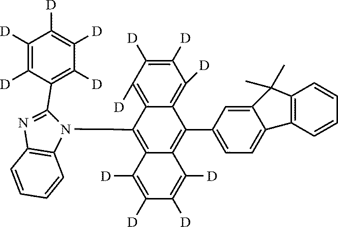 [SMILES: [2H]c1c([2H])c([2H])c(-c2nc3ccccc3n2-c2c3c([2H])c([2H])c([2H])c([2H])c3c(-c3ccc4c(c3)C(C)(C)c3ccccc3-4)c3c([2H])c([2H])c([2H])c([2H])c23)c([2H])c1[2H]]

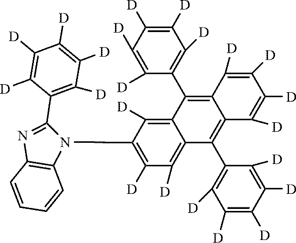 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-n4c(-c5c([2H])c([2H])c([2H])c([2H])c5[2H])nc5ccccc54)c([2H])c([2H])c23)c([2H])c1[2H]]

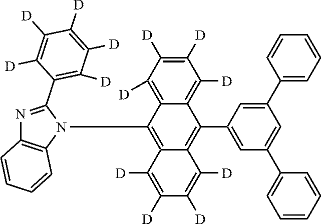 [SMILES: [2H]c1c([2H])c([2H])c(-c2nc3ccccc3n2-c2c3c([2H])c([2H])c([2H])c([2H])c3c(-c3cc(-c4ccccc4)cc(-c4ccccc4)c3)c3c([2H])c([2H])c([2H])c([2H])c23)c([2H])c1[2H]]

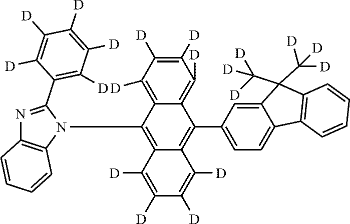 [SMILES: [2H]c1c([2H])c([2H])c(-c2nc3ccccc3n2-c2c3c([2H])c([2H])c([2H])c([2H])c3c(-c3ccc4c(c3)C(C([2H])([2H])[2H])(C([2H])([2H])[2H])c3ccccc3-4)c3c([2H])c([2H])c([2H])c([2H])c23)c([2H])c1[2H]]

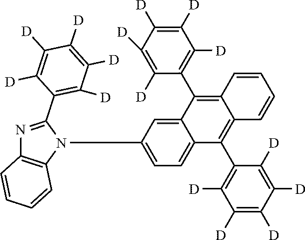 [SMILES: [2H]c1c([2H])c([2H])c(-c2c3ccccc3c(-c3c([2H])c([2H])c([2H])c([2H])c3[2H])c3cc(-n4c(-c5c([2H])c([2H])c([2H])c([2H])c5[2H])nc5ccccc54)ccc23)c([2H])c1[2H]]

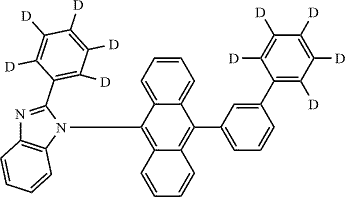 [SMILES: [2H]c1c([2H])c([2H])c(-c2cccc(-c3c4ccccc4c(-n4c(-c5c([2H])c([2H])c([2H])c([2H])c5[2H])nc5ccccc54)c4ccccc34)c2)c([2H])c1[2H]]

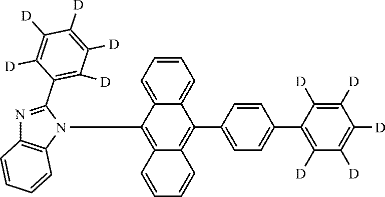 [SMILES: [2H]c1c([2H])c([2H])c(-c2ccc(-c3c4ccccc4c(-n4c(-c5c([2H])c([2H])c([2H])c([2H])c5[2H])nc5ccccc54)c4ccccc34)cc2)c([2H])c1[2H]]

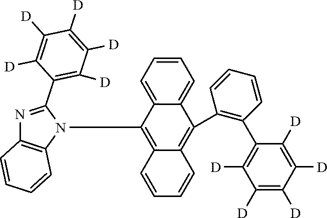 [SMILES: [2H]c1c([2H])c([2H])c(-c2ccccc2-c2c3ccccc3c(-n3c(-c4c([2H])c([2H])c([2H])c([2H])c4[2H])nc4ccccc43)c3ccccc23)c([2H])c1[2H]]

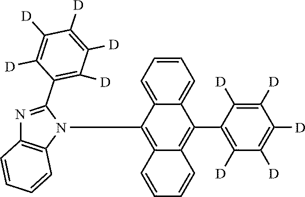 [SMILES: [2H]c1c([2H])c([2H])c(-c2c3ccccc3c(-n3c(-c4c([2H])c([2H])c([2H])c([2H])c4[2H])nc4ccccc43)c3ccccc23)c([2H])c1[2H]]

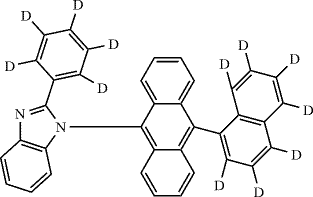 [SMILES: [2H]c1c([2H])c([2H])c(-c2nc3ccccc3n2-c2c3ccccc3c(-c3c([2H])c([2H])c([2H])c4c([2H])c([2H])c([2H])c([2H])c34)c3ccccc23)c([2H])c1[2H]]

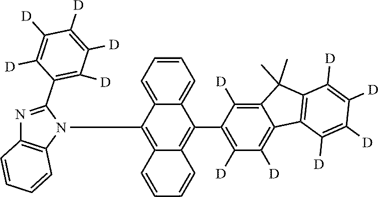 [SMILES: [2H]c1c([2H])c([2H])c(-c2nc3ccccc3n2-c2c3ccccc3c(-c3c([2H])c([2H])c4c(c3[2H])C(C)(C)c3c([2H])c([2H])c([2H])c([2H])c3-4)c3ccccc23)c([2H])c1[2H]]

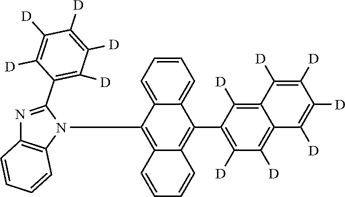 [SMILES: [2H]c1c([2H])c([2H])c(-c2nc3ccccc3n2-c2c3ccccc3c(-c3c([2H])c([2H])c4c([2H])c([2H])c([2H])c([2H])c4c3[2H])c3ccccc23)c([2H])c1[2H]]

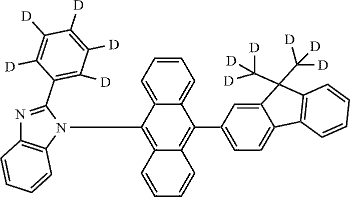 [SMILES: [2H]c1c([2H])c([2H])c(-c2nc3ccccc3n2-c2c3ccccc3c(-c3ccc4c(c3)C(C([2H])([2H])[2H])(C([2H])([2H])[2H])c3ccccc3-4)c3ccccc23)c([2H])c1[2H]]

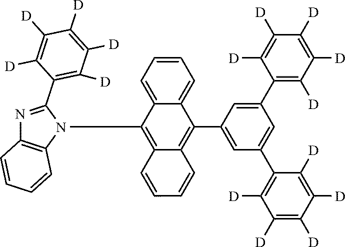 [SMILES: [2H]c1c([2H])c([2H])c(-c2cc(-c3c([2H])c([2H])c([2H])c([2H])c3[2H])cc(-c3c4ccccc4c(-n4c(-c5c([2H])c([2H])c([2H])c([2H])c5[2H])nc5ccccc54)c4ccccc34)c2)c([2H])c1[2H]]

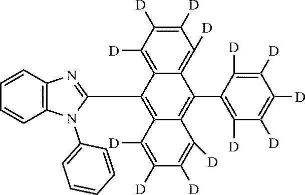 [SMILES: [2H]c1c([2H])c([2H])c(-c2c3c([2H])c([2H])c([2H])c([2H])c3c(-c3nc4ccccc4n3-c3ccccc3)c3c([2H])c([2H])c([2H])c([2H])c23)c([2H])c1[2H]]

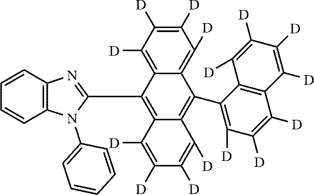 [SMILES: [2H]c1c([2H])c([2H])c2c(-c3c4c([2H])c([2H])c([2H])c([2H])c4c(-c4nc5ccccc5n4-c4ccccc4)c4c([2H])c([2H])c([2H])c([2H])c34)c([2H])c([2H])c([2H])c2c1[2H]]

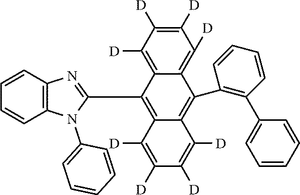 [SMILES: [2H]c1c([2H])c([2H])c2c(-c3nc4ccccc4n3-c3ccccc3)c3c([2H])c([2H])c([2H])c([2H])c3c(-c3ccccc3-c3ccccc3)c2c1[2H]]

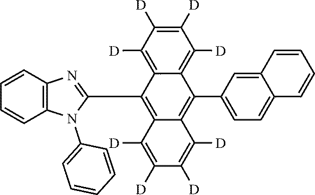 [SMILES: [2H]c1c([2H])c([2H])c2c(-c3nc4ccccc4n3-c3ccccc3)c3c([2H])c([2H])c([2H])c([2H])c3c(-c3ccc4ccccc4c3)c2c1[2H]]

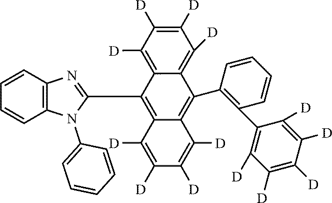 [SMILES: [2H]c1c([2H])c([2H])c(-c2ccccc2-c2c3c([2H])c([2H])c([2H])c([2H])c3c(-c3nc4ccccc4n3-c3ccccc3)c3c([2H])c([2H])c([2H])c([2H])c23)c([2H])c1[2H]]

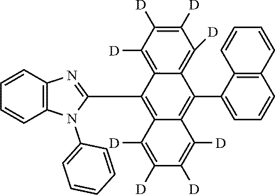 [SMILES: [2H]c1c([2H])c([2H])c2c(-c3nc4ccccc4n3-c3ccccc3)c3c([2H])c([2H])c([2H])c([2H])c3c(-c3cccc4ccccc34)c2c1[2H]]

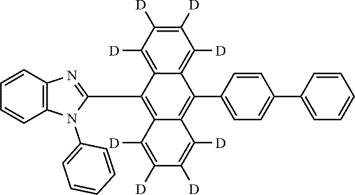 [SMILES: [2H]c1c([2H])c([2H])c2c(-c3nc4ccccc4n3-c3ccccc3)c3c([2H])c([2H])c([2H])c([2H])c3c(-c3ccc(-c4ccccc4)cc3)c2c1[2H]]

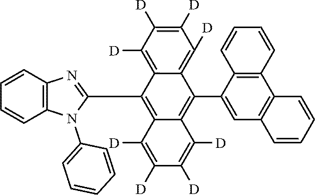 [SMILES: [2H]c1c([2H])c([2H])c2c(-c3nc4ccccc4n3-c3ccccc3)c3c([2H])c([2H])c([2H])c([2H])c3c(-c3cc4ccccc4c4ccccc34)c2c1[2H]]

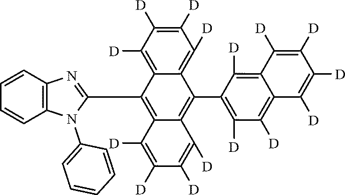 [SMILES: [2H]c1c([2H])c([2H])c2c([2H])c(-c3c4c([2H])c([2H])c([2H])c([2H])c4c(-c4nc5ccccc5n4-c4ccccc4)c4c([2H])c([2H])c([2H])c([2H])c34)c([2H])c([2H])c2c1[2H]]

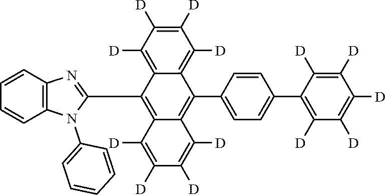 [SMILES: [2H]c1c([2H])c([2H])c(-c2ccc(-c3c4c([2H])c([2H])c([2H])c([2H])c4c(-c4nc5ccccc5n4-c4ccccc4)c4c([2H])c([2H])c([2H])c([2H])c34)cc2)c([2H])c1[2H]]

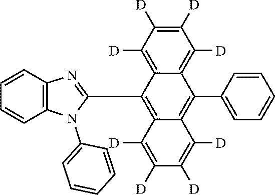 [SMILES: [2H]c1c([2H])c([2H])c2c(-c3nc4ccccc4n3-c3ccccc3)c3c([2H])c([2H])c([2H])c([2H])c3c(-c3ccccc3)c2c1[2H]]

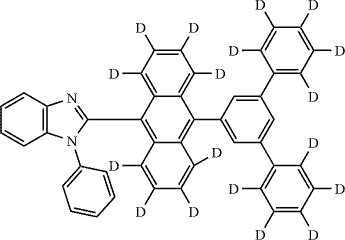 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-c4nc5ccccc5n4-c4ccccc4)c4c([2H])c([2H])c([2H])c([2H])c34)c2)c([2H])c1[2H]]

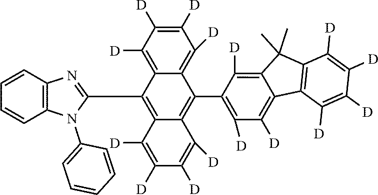 [SMILES: [2H]c1c([2H])c([2H])c2c(c1[2H])-c1c([2H])c([2H])c(-c3c4c([2H])c([2H])c([2H])c([2H])c4c(-c4nc5ccccc5n4-c4ccccc4)c4c([2H])c([2H])c([2H])c([2H])c34)c([2H])c1C2(C)C]

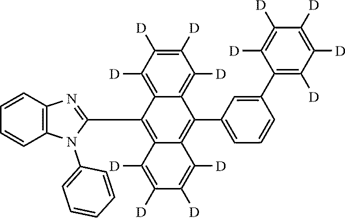

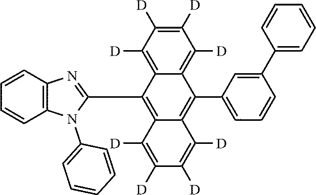 [SMILES: [2H]c1c([2H])c([2H])c2c(-c3nc4ccccc4n3-c3ccccc3)c3c([2H])c([2H])c([2H])c([2H])c3c(-c3cccc(-c4ccccc4)c3)c2c1[2H]]

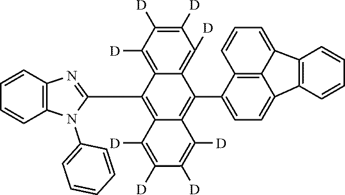 [SMILES: [2H]c1c([2H])c([2H])c2c(-c3nc4ccccc4n3-c3ccccc3)c3c([2H])c([2H])c([2H])c([2H])c3c(-c3ccc4c5c(cccc35)-c3ccccc3-4)c2c1[2H]]

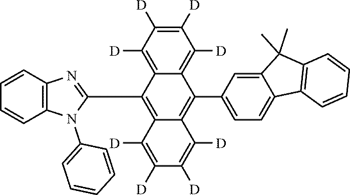 [SMILES: [2H]c1c([2H])c([2H])c2c(-c3nc4ccccc4n3-c3ccccc3)c3c([2H])c([2H])c([2H])c([2H])c3c(-c3ccc4c(c3)C(C)(C)c3ccccc3-4)c2c1[2H]]

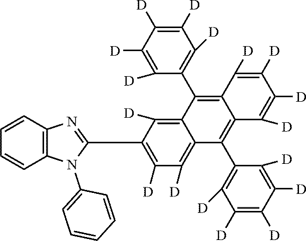 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4nc5ccccc5n4-c4ccccc4)c([2H])c([2H])c23)c([2H])c1[2H]]

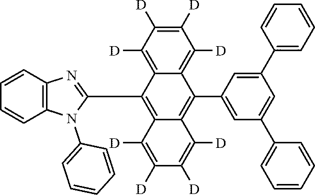 [SMILES: [2H]c1c([2H])c([2H])c2c(-c3nc4ccccc4n3-c3ccccc3)c3c([2H])c([2H])c([2H])c([2H])c3c(-c3cc(-c4ccccc4)cc(-c4ccccc4)c3)c2c1[2H]]

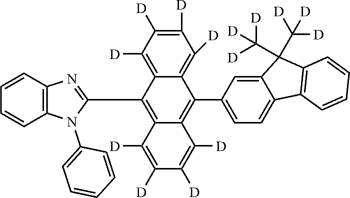 [SMILES: [2H]c1c([2H])c([2H])c2c(-c3nc4ccccc4n3-c3ccccc3)c3c([2H])c([2H])c([2H])c([2H])c3c(-c3ccc4c(c3)C(C([2H])([2H])[2H])(C([2H])([2H])[2H])c3ccccc3-4)c2c1[2H]]

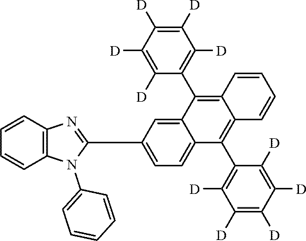 [SMILES: [2H]c1c([2H])c([2H])c(-c2c3ccccc3c(-c3c([2H])c([2H])c([2H])c([2H])c3[2H])c3cc(-c4nc5ccccc5n4-c4ccccc4)ccc23)c([2H])c1[2H]]

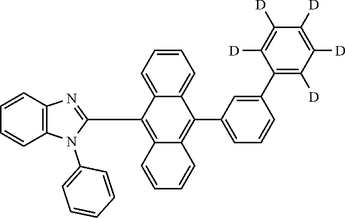 [SMILES: [2H]c1c([2H])c([2H])c(-c2cccc(-c3c4ccccc4c(-c4nc5ccccc5n4-c4ccccc4)c4ccccc34)c2)c([2H])c1[2H]]

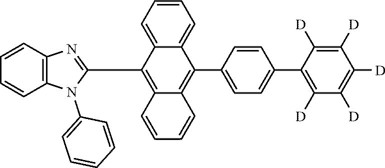 [SMILES: [2H]c1c([2H])c([2H])c(-c2ccc(-c3c4ccccc4c(-c4nc5ccccc5n4-c4ccccc4)c4ccccc34)cc2)c([2H])c1[2H]]

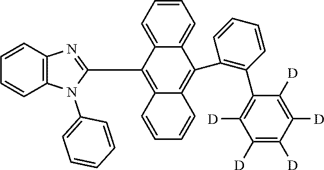 [SMILES: [2H]c1c([2H])c([2H])c(-c2ccccc2-c2c3ccccc3c(-c3nc4ccccc4n3-c3ccccc3)c3ccccc23)c([2H])c1[2H]]

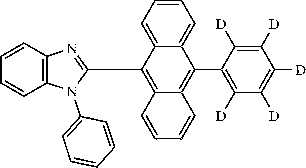 [SMILES: [2H]c1c([2H])c([2H])c(-c2c3ccccc3c(-c3nc4ccccc4n3-c3ccccc3)c3ccccc23)c([2H])c1[2H]]

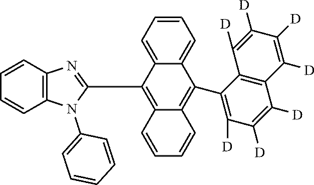 [SMILES: [2H]c1c([2H])c([2H])c2c(-c3c4ccccc4c(-c4nc5ccccc5n4-c4ccccc4)c4ccccc34)c([2H])c([2H])c([2H])c2c1[2H]]

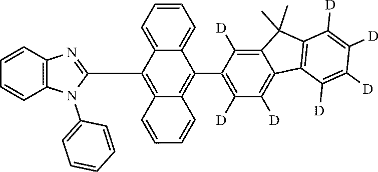 [SMILES: [2H]c1c([2H])c([2H])c2c(c1[2H])-c1c([2H])c([2H])c(-c3c4ccccc4c(-c4nc5ccccc5n4-c4ccccc4)c4ccccc34)c([2H])c1C2(C)C]

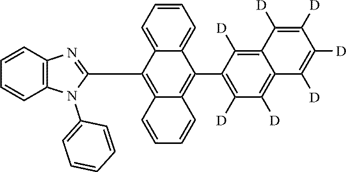 [SMILES: [2H]c1c([2H])c([2H])c2c([2H])c(-c3c4ccccc4c(-c4nc5ccccc5n4-c4ccccc4)c4ccccc34)c([2H])c([2H])c2c1[2H]]

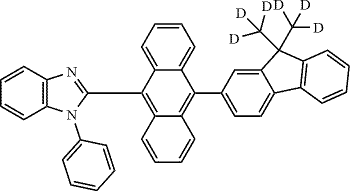 [SMILES: [2H]C([2H])([2H])C1(C([2H])([2H])[2H])c2ccccc2-c2ccc(-c3c4ccccc4c(-c4nc5ccccc5n4-c4ccccc4)c4ccccc34)cc21]

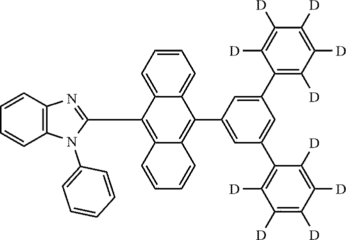 [SMILES: [2H]c1c([2H])c([2H])c(-c2cc(-c3c([2H])c([2H])c([2H])c([2H])c3[2H])cc(-c3c4ccccc4c(-c4nc5ccccc5n4-c4ccccc4)c4ccccc34)c2)c([2H])c1[2H]]

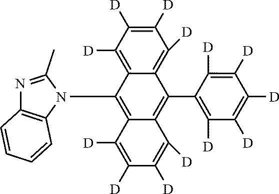 [SMILES: [2H]c1c([2H])c([2H])c(-c2c3c([2H])c([2H])c([2H])c([2H])c3c(-n3c(C)nc4ccccc43)c3c([2H])c([2H])c([2H])c([2H])c23)c([2H])c1[2H]]

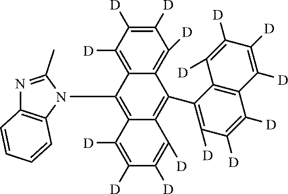 [SMILES: [2H]c1c([2H])c([2H])c2c(-c3c4c([2H])c([2H])c([2H])c([2H])c4c(-n4c(C)nc5ccccc54)c4c([2H])c([2H])c([2H])c([2H])c34)c([2H])c([2H])c([2H])c2c1[2H]]

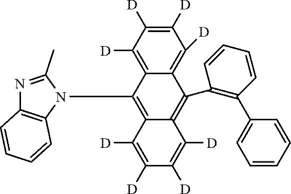 [SMILES: [2H]c1c([2H])c([2H])c2c(-n3c(C)nc4ccccc43)c3c([2H])c([2H])c([2H])c([2H])c3c(-c3ccccc3-c3ccccc3)c2c1[2H]]

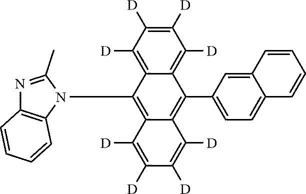 [SMILES: [2H]c1c([2H])c([2H])c2c(-n3c(C)nc4ccccc43)c3c([2H])c([2H])c([2H])c([2H])c3c(-c3ccc4ccccc4c3)c2c1[2H]]

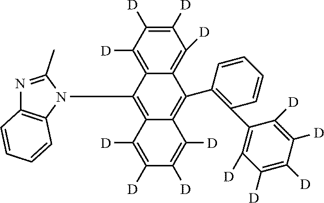 [SMILES: [2H]c1c([2H])c([2H])c(-c2ccccc2-c2c3c([2H])c([2H])c([2H])c([2H])c3c(-n3c(C)nc4ccccc43)c3c([2H])c([2H])c([2H])c([2H])c23)c([2H])c1[2H]]

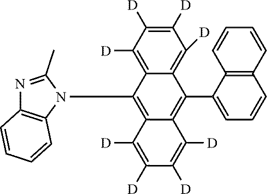 [SMILES: [2H]c1c([2H])c([2H])c2c(-n3c(C)nc4ccccc43)c3c([2H])c([2H])c([2H])c([2H])c3c(-c3cccc4ccccc34)c2c1[2H]]

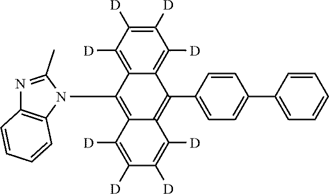 [SMILES: [2H]c1c([2H])c([2H])c2c(-n3c(C)nc4ccccc43)c3c([2H])c([2H])c([2H])c([2H])c3c(-c3ccc(-c4ccccc4)cc3)c2c1[2H]]

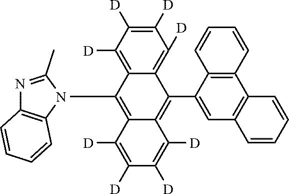 [SMILES: [2H]c1c([2H])c([2H])c2c(-n3c(C)nc4ccccc43)c3c([2H])c([2H])c([2H])c([2H])c3c(-c3cc4ccccc4c4ccccc34)c2c1[2H]]

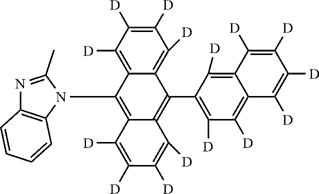 [SMILES: [2H]c1c([2H])c([2H])c2c([2H])c(-c3c4c([2H])c([2H])c([2H])c([2H])c4c(-n4c(C)nc5ccccc54)c4c([2H])c([2H])c([2H])c([2H])c34)c([2H])c([2H])c2c1[2H]]

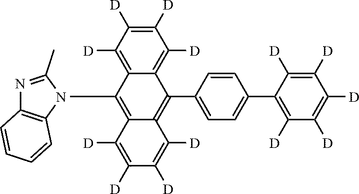 [SMILES: [2H]c1c([2H])c([2H])c(-c2ccc(-c3c4c([2H])c([2H])c([2H])c([2H])c4c(-n4c(C)nc5ccccc54)c4c([2H])c([2H])c([2H])c([2H])c34)cc2)c([2H])c1[2H]]

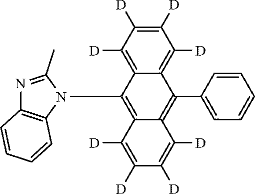 [SMILES: [2H]c1c([2H])c([2H])c2c(-n3c(C)nc4ccccc43)c3c([2H])c([2H])c([2H])c([2H])c3c(-c3ccccc3)c2c1[2H]]

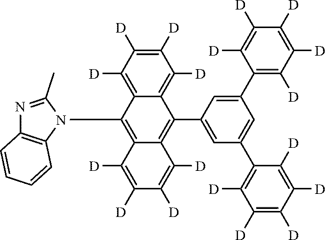 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(C)nc5ccccc54)c4c([2H])c([2H])c([2H])c([2H])c34)c2)c([2H])c1[2H]]

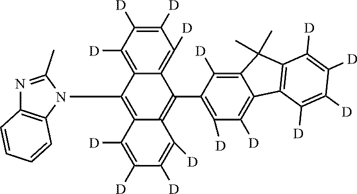 [SMILES: [2H]c1c([2H])c([2H])c2c(c1[2H])-c1c([2H])c([2H])c(-c3c4c([2H])c([2H])c([2H])c([2H])c4c(-n4c(C)nc5ccccc54)c4c([2H])c([2H])c([2H])c([2H])c34)c([2H])c1C2(C)C]

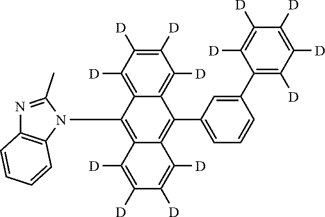 [SMILES: [2H]c1c([2H])c([2H])c(-c2cccc(-c3c4c([2H])c([2H])c([2H])c([2H])c4c(-n4c(C)nc5ccccc54)c4c([2H])c([2H])c([2H])c([2H])c34)c2)c([2H])c1[2H]]

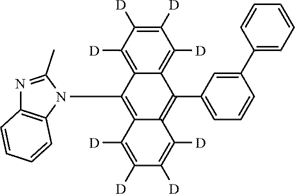 [SMILES: [2H]c1c([2H])c([2H])c2c(-n3c(C)nc4ccccc43)c3c([2H])c([2H])c([2H])c([2H])c3c(-c3cccc(-c4ccccc4)c3)c2c1[2H]]

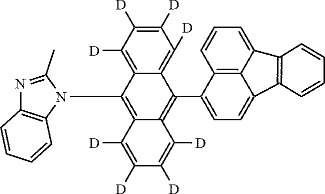 [SMILES: [2H]c1c([2H])c([2H])c2c(-n3c(C)nc4ccccc43)c3c([2H])c([2H])c([2H])c([2H])c3c(-c3ccc4c5c(cccc35)-c3ccccc3-4)c2c1[2H]]

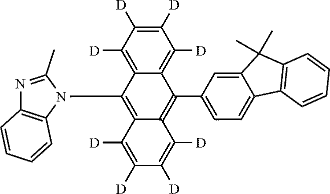 [SMILES: [2H]c1c([2H])c([2H])c2c(-n3c(C)nc4ccccc43)c3c([2H])c([2H])c([2H])c([2H])c3c(-c3ccc4c(c3)C(C)(C)c3ccccc3-4)c2c1[2H]]

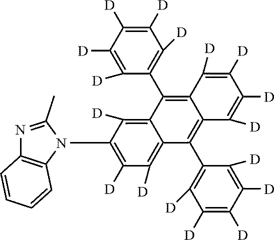 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-n4c(C)nc5ccccc54)c([2H])c([2H])c23)c([2H])c1[2H]]

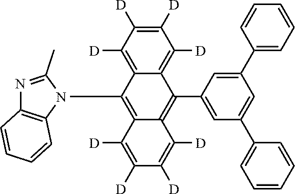 [SMILES: [2H]c1c([2H])c([2H])c2c(-n3c(C)nc4ccccc43)c3c([2H])c([2H])c([2H])c([2H])c3c(-c3cc(-c4ccccc4)cc(-c4ccccc4)c3)c2c1[2H]]

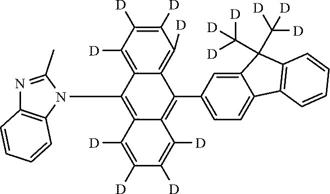 [SMILES: [2H]c1c([2H])c([2H])c2c(-n3c(C)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

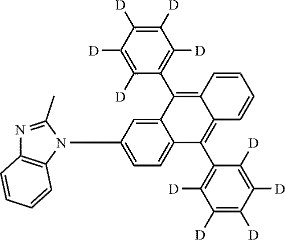 [SMILES: [2H]c1c([2H])c([2H])c(-c2c3ccccc3c(-c3c([2H])c([2H])c([2H])c([2H])c3[2H])c3cc(-n4c(C)nc5ccccc54)ccc23)c([2H])c1[2H]]

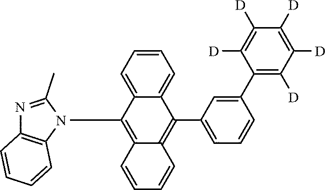 [SMILES: [2H]c1c([2H])c([2H])c(-c2cccc(-c3c4ccccc4c(-n4c(C)nc5ccccc54)c4ccccc34)c2)c([2H])c1[2H]]

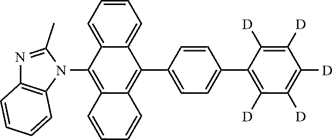 [SMILES: [2H]c1c([2H])c([2H])c(-c2ccc(-c3c4ccccc4c(-n4c(C)nc5ccccc54)c4ccccc34)cc2)c([2H])c1[2H]]

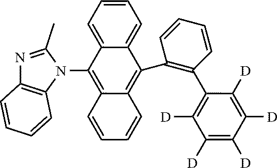 [SMILES: [2H]c1c([2H])c([2H])c(-c2ccccc2-c2c3ccccc3c(-n3c(C)nc4ccccc43)c3ccccc23)c([2H])c1[2H]]

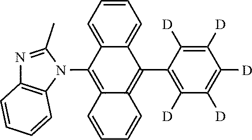 [SMILES: [2H]c1c([2H])c([2H])c(-c2c3ccccc3c(-n3c(C)nc4ccccc43)c3ccccc23)c([2H])c1[2H]]

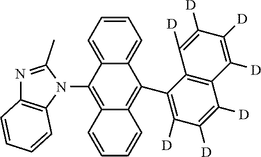 [SMILES: [2H]c1c([2H])c([2H])c2c(-c3c4ccccc4c(-n4c(C)nc5ccccc54)c4ccccc34)c([2H])c([2H])c([2H])c2c1[2H]]

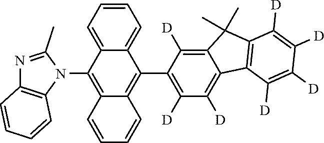 [SMILES: [2H]c1c([2H])c([2H])c2c(c1[2H])-c1c([2H])c([2H])c(-c3c4ccccc4c(-n4c(C)nc5ccccc54)c4ccccc34)c([2H])c1C2(C)C]

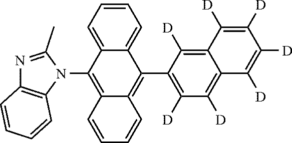 [SMILES: [2H]c1c([2H])c([2H])c2c([2H])c(-c3c4ccccc4c(-n4c(C)nc5ccccc54)c4ccccc34)c([2H])c([2H])c2c1[2H]]

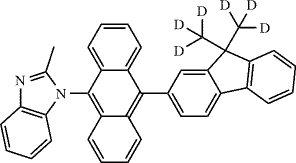 [SMILES: [2H]C([2H])([2H])C1(C([2H])([2H])[2H])c2ccccc2-c2ccc(-c3c4ccccc4c(-n4c(C)nc5ccccc54)c4ccccc34)cc21]

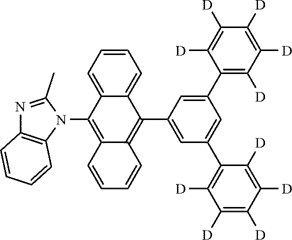 [SMILES: [2H]c1c([2H])c([2H])c(-c2cc(-c3c([2H])c([2H])c([2H])c([2H])c3[2H])cc(-c3c4ccccc4c(-n4c(C)nc5ccccc54)c4ccccc34)c2)c([2H])c1[2H]]

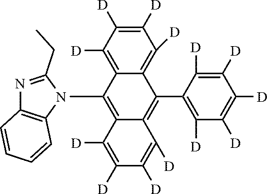 [SMILES: [2H]c1c([2H])c([2H])c(-c2c3c([2H])c([2H])c([2H])c([2H])c3c(-n3c(CC)nc4ccccc43)c3c([2H])c([2H])c([2H])c([2H])c23)c([2H])c1[2H]]

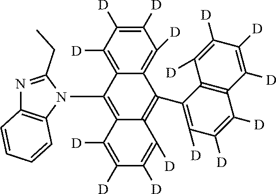 [SMILES: [2H]c1c([2H])c([2H])c2c(-c3c4c([2H])c([2H])c([2H])c([2H])c4c(-n4c(CC)nc5ccccc54)c4c([2H])c([2H])c([2H])c([2H])c34)c([2H])c([2H])c([2H])c2c1[2H]]

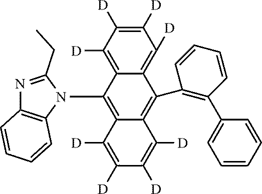 [SMILES: [2H]c1c([2H])c([2H])c2c(-n3c(CC)nc4ccccc43)c3c([2H])c([2H])c([2H])c([2H])c3c(-c3ccccc3-c3ccccc3)c2c1[2H]]

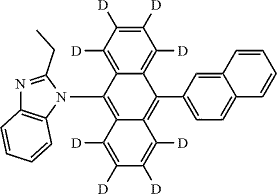 [SMILES: [2H]c1c([2H])c([2H])c2c(-n3c(CC)nc4ccccc43)c3c([2H])c([2H])c([2H])c([2H])c3c(-c3ccc4ccccc4c3)c2c1[2H]]

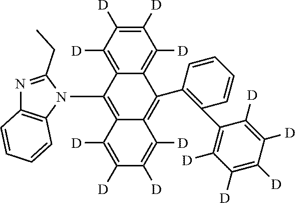 [SMILES: [2H]c1c([2H])c([2H])c(-c2ccccc2-c2c3c([2H])c([2H])c([2H])c([2H])c3c(-n3c(CC)nc4ccccc43)c3c([2H])c([2H])c([2H])c([2H])c23)c([2H])c1[2H]]

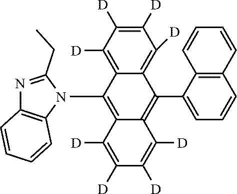 [SMILES: [2H]c1c([2H])c([2H])c2c(-n3c(CC)nc4ccccc43)c3c([2H])c([2H])c([2H])c([2H])c3c(-c3cccc4ccccc34)c2c1[2H]]

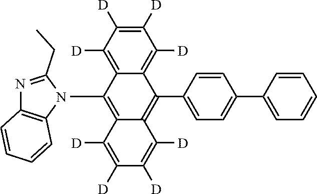 [SMILES: [2H]c1c([2H])c([2H])c2c(-n3c(CC)nc4ccccc43)c3c([2H])c([2H])c([2H])c([2H])c3c(-c3ccc(-c4ccccc4)cc3)c2c1[2H]]

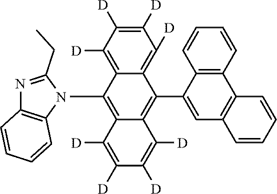 [SMILES: [2H]c1c([2H])c([2H])c2c(-n3c(CC)nc4ccccc43)c3c([2H])c([2H])c([2H])c([2H])c3c(-c3cc4ccccc4c4ccccc34)c2c1[2H]]

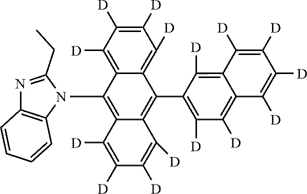 [SMILES: [2H]c1c([2H])c([2H])c2c([2H])c(-c3c4c([2H])c([2H])c([2H])c([2H])c4c(-n4c(CC)nc5ccccc54)c4c([2H])c([2H])c([2H])c([2H])c34)c([2H])c([2H])c2c1[2H]]

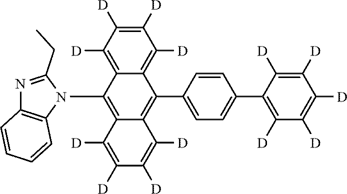 [SMILES: [2H]c1c([2H])c([2H])c(-c2ccc(-c3c4c([2H])c([2H])c([2H])c([2H])c4c(-n4c(CC)nc5ccccc54)c4c([2H])c([2H])c([2H])c([2H])c34)cc2)c([2H])c1[2H]]

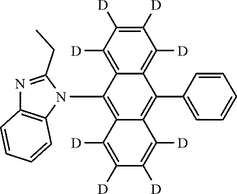 [SMILES: [2H]c1c([2H])c([2H])c2c(-n3c(CC)nc4ccccc43)c3c([2H])c([2H])c([2H])c([2H])c3c(-c3ccccc3)c2c1[2H]]

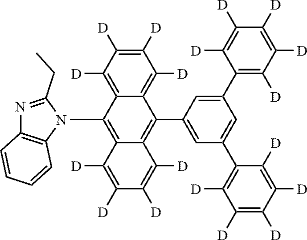 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(CC)nc5ccccc54)c4c([2H])c([2H])c([2H])c([2H])c34)c2)c([2H])c1[2H]]

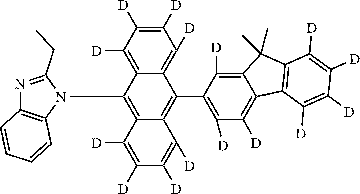 [SMILES: [2H]c1c([2H])c([2H])c2c(c1[2H])-c1c([2H])c([2H])c(-c3c4c([2H])c([2H])c([2H])c([2H])c4c(-n4c(CC)nc5ccccc54)c4c([2H])c([2H])c([2H])c([2H])c34)c([2H])c1C2(C)C]

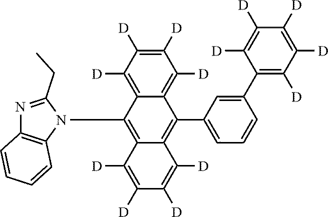 [SMILES: [2H]c1c([2H])c([2H])c(-c2cccc(-c3c4c([2H])c([2H])c([2H])c([2H])c4c(-n4c(CC)nc5ccccc54)c4c([2H])c([2H])c([2H])c([2H])c34)c2)c([2H])c1[2H]]

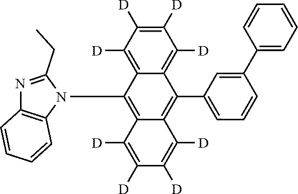 [SMILES: [2H]c1c([2H])c([2H])c2c(-n3c(CC)nc4ccccc43)c3c([2H])c([2H])c([2H])c([2H])c3c(-c3cccc(-c4ccccc4)c3)c2c1[2H]]

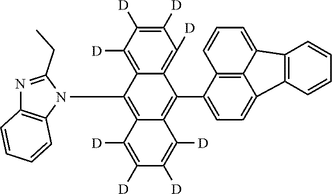 [SMILES: [2H]c1c([2H])c([2H])c2c(-n3c(CC)nc4ccccc43)c3c([2H])c([2H])c([2H])c([2H])c3c(-c3ccc4c5c(cccc35)-c3ccccc3-4)c2c1[2H]]

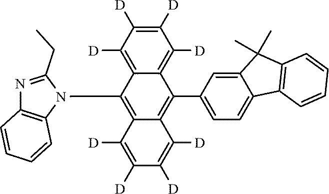 [SMILES: [2H]c1c([2H])c([2H])c2c(-n3c(CC)nc4ccccc43)c3c([2H])c([2H])c([2H])c([2H])c3c(-c3ccc4c(c3)C(C)(C)c3ccccc3-4)c2c1[2H]]

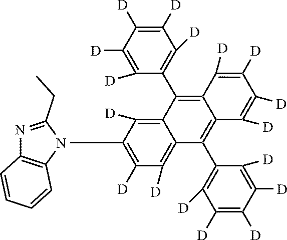 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-n4c(CC)nc5ccccc54)c([2H])c([2H])c23)c([2H])c1[2H]]

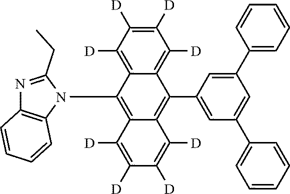 [SMILES: [2H]c1c([2H])c([2H])c2c(-n3c(CC)nc4ccccc43)c3c([2H])c([2H])c([2H])c([2H])c3c(-c3cc(-c4ccccc4)cc(-c4ccccc4)c3)c2c1[2H]]

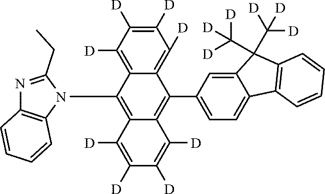 [SMILES: [2H]c1c([2H])c([2H])c2c(-n3c(CC)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

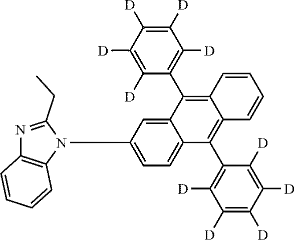 [SMILES: [2H]c1c([2H])c([2H])c(-c2c3ccccc3c(-c3c([2H])c([2H])c([2H])c([2H])c3[2H])c3cc(-n4c(CC)nc5ccccc54)ccc23)c([2H])c1[2H]]

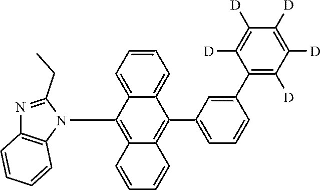 [SMILES: [2H]c1c([2H])c([2H])c(-c2cccc(-c3c4ccccc4c(-n4c(CC)nc5ccccc54)c4ccccc34)c2)c([2H])c1[2H]]

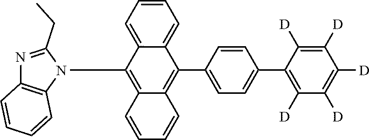 [SMILES: [2H]c1c([2H])c([2H])c(-c2ccc(-c3c4ccccc4c(-n4c(CC)nc5ccccc54)c4ccccc34)cc2)c([2H])c1[2H]]

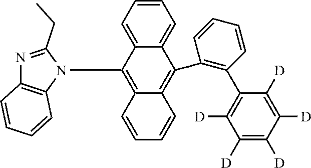 [SMILES: [2H]c1c([2H])c([2H])c(-c2ccccc2-c2c3ccccc3c(-n3c(CC)nc4ccccc43)c3ccccc23)c([2H])c1[2H]]

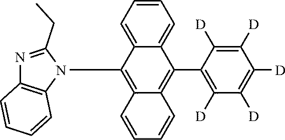 [SMILES: [2H]c1c([2H])c([2H])c(-c2c3ccccc3c(-n3c(CC)nc4ccccc43)c3ccccc23)c([2H])c1[2H]]

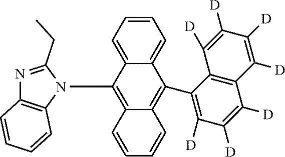 [SMILES: [2H]c1c([2H])c([2H])c2c(-c3c4ccccc4c(-n4c(CC)nc5ccccc54)c4ccccc34)c([2H])c([2H])c([2H])c2c1[2H]]

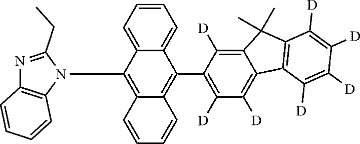 [SMILES: [2H]c1c([2H])c([2H])c2c(c1[2H])-c1c([2H])c([2H])c(-c3c4ccccc4c(-n4c(CC)nc5ccccc54)c4ccccc34)c([2H])c1C2(C)C]

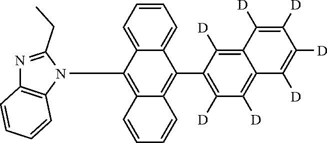 [SMILES: [2H]c1c([2H])c([2H])c2c([2H])c(-c3c4ccccc4c(-n4c(CC)nc5ccccc54)c4ccccc34)c([2H])c([2H])c2c1[2H]]

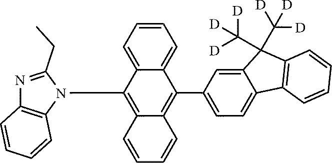 [SMILES: [2H]C([2H])([2H])C1(C([2H])([2H])[2H])c2ccccc2-c2ccc(-c3c4ccccc4c(-n4c(CC)nc5ccccc54)c4ccccc34)cc21]

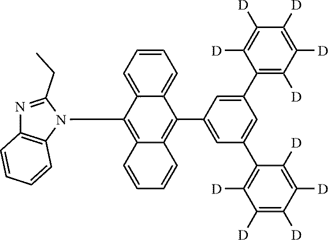 [SMILES: [2H]c1c([2H])c([2H])c(-c2cc(-c3c([2H])c([2H])c([2H])c([2H])c3[2H])cc(-c3c4ccccc4c(-n4c(CC)nc5ccccc54)c4ccccc34)c2)c([2H])c1[2H]]

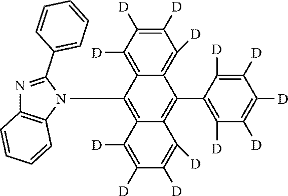 [SMILES: [2H]c1c([2H])c([2H])c(-c2c3c([2H])c([2H])c([2H])c([2H])c3c(-n3c(-c4ccccc4)nc4ccccc43)c3c([2H])c([2H])c([2H])c([2H])c23)c([2H])c1[2H]]

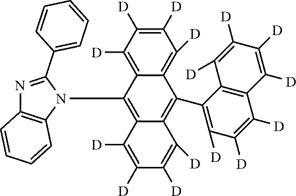 [SMILES: [2H]c1c([2H])c([2H])c2c(-c3c4c([2H])c([2H])c([2H])c([2H])c4c(-n4c(-c5ccccc5)nc5ccccc54)c4c([2H])c([2H])c([2H])c([2H])c34)c([2H])c([2H])c([2H])c2c1[2H]]

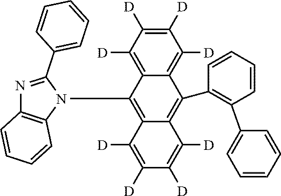 [SMILES: [2H]c1c([2H])c([2H])c2c(-n3c(-c4ccccc4)nc4ccccc43)c3c([2H])c([2H])c([2H])c([2H])c3c(-c3ccccc3-c3ccccc3)c2c1[2H]]

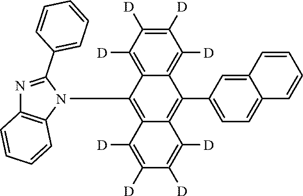 [SMILES: [2H]c1c([2H])c([2H])c2c(-n3c(-c4ccccc4)nc4ccccc43)c3c([2H])c([2H])c([2H])c([2H])c3c(-c3ccc4ccccc4c3)c2c1[2H]]

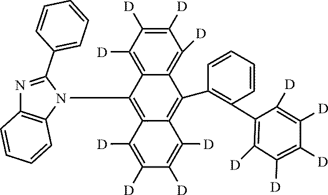 [SMILES: [2H]c1c([2H])c([2H])c(-c2ccccc2-c2c3c([2H])c([2H])c([2H])c([2H])c3c(-n3c(-c4ccccc4)nc4ccccc43)c3c([2H])c([2H])c([2H])c([2H])c23)c([2H])c1[2H]]

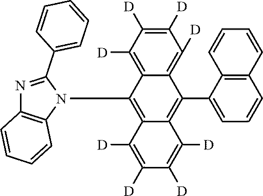 [SMILES: [2H]c1c([2H])c([2H])c2c(-n3c(-c4ccccc4)nc4ccccc43)c3c([2H])c([2H])c([2H])c([2H])c3c(-c3cccc4ccccc34)c2c1[2H]]

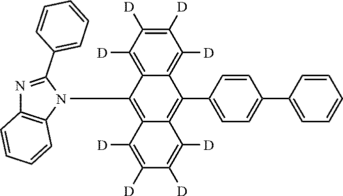 [SMILES: [2H]c1c([2H])c([2H])c2c(-n3c(-c4ccccc4)nc4ccccc43)c3c([2H])c([2H])c([2H])c([2H])c3c(-c3ccc(-c4ccccc4)cc3)c2c1[2H]]

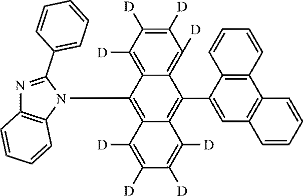 [SMILES: [2H]c1c([2H])c([2H])c2c(-n3c(-c4ccccc4)nc4ccccc43)c3c([2H])c([2H])c([2H])c([2H])c3c(-c3cc4ccccc4c4ccccc34)c2c1[2H]]

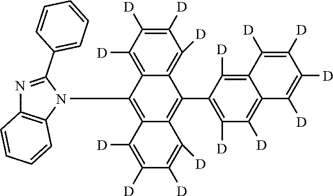 [SMILES: [2H]c1c([2H])c([2H])c2c([2H])c(-c3c4c([2H])c([2H])c([2H])c([2H])c4c(-n4c(-c5ccccc5)nc5ccccc54)c4c([2H])c([2H])c([2H])c([2H])c34)c([2H])c([2H])c2c1[2H]]

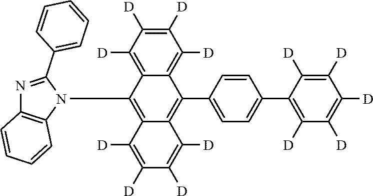 [SMILES: [2H]c1c([2H])c([2H])c(-c2ccc(-c3c4c([2H])c([2H])c([2H])c([2H])c4c(-n4c(-c5ccccc5)nc5ccccc54)c4c([2H])c([2H])c([2H])c([2H])c34)cc2)c([2H])c1[2H]]

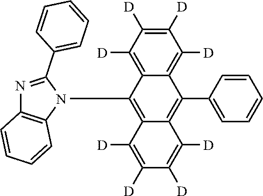 [SMILES: [2H]c1c([2H])c([2H])c2c(-n3c(-c4ccccc4)nc4ccccc43)c3c([2H])c([2H])c([2H])c([2H])c3c(-c3ccccc3)c2c1[2H]]

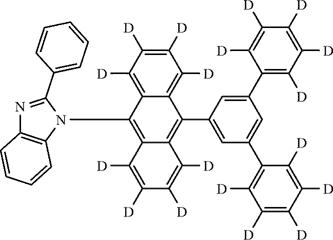 [SMILES: [2H]c1c([2H])c([2H])c(-c2cc(-c3c([2H])c([2H])c([2H])c([2H])c3[2H])cc(-c3c4c([2H])c([2H])c([2H])c([2H])c4c(-n4c(-c5ccccc5)nc5ccccc54)c4c([2H])c([2H])c([2H])c([2H])c34)c2)c([2H])c1[2H]]

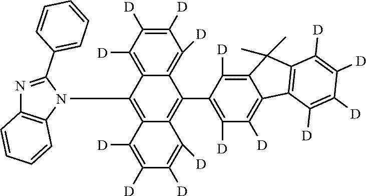 [SMILES: [2H]c1c([2H])c([2H])c2c(c1[2H])-c1c([2H])c([2H])c(-c3c4c([2H])c([2H])c([2H])c([2H])c4c(-n4c(-c5ccccc5)nc5ccccc54)c4c([2H])c([2H])c([2H])c([2H])c34)c([2H])c1C2(C)C]

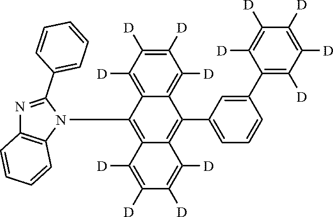 [SMILES: [2H]c1c([2H])c([2H])c(-c2cccc(-c3c4c([2H])c([2H])c([2H])c([2H])c4c(-n4c(-c5ccccc5)nc5ccccc54)c4c([2H])c([2H])c([2H])c([2H])c34)c2)c([2H])c1[2H]]

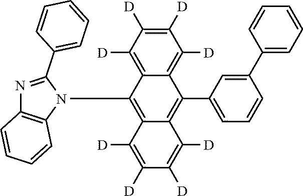 [SMILES: [2H]c1c([2H])c([2H])c2c(-n3c(-c4ccccc4)nc4ccccc43)c3c([2H])c([2H])c([2H])c([2H])c3c(-c3cccc(-c4ccccc4)c3)c2c1[2H]]

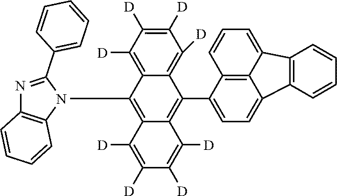 [SMILES: [2H]c1c([2H])c([2H])c2c(-n3c(-c4ccccc4)nc4ccccc43)c3c([2H])c([2H])c([2H])c([2H])c3c(-c3ccc4c5c(cccc35)-c3ccccc3-4)c2c1[2H]]

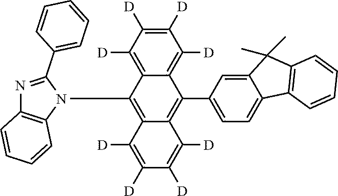 [SMILES: [2H]c1c([2H])c([2H])c2c(-n3c(-c4ccccc4)nc4ccccc43)c3c([2H])c([2H])c([2H])c([2H])c3c(-c3ccc4c(c3)C(C)(C)c3ccccc3-4)c2c1[2H]]

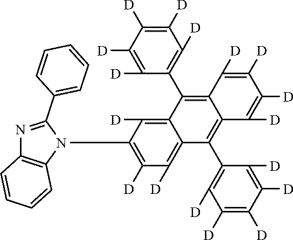 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-n4c(-c5ccccc5)nc5ccccc54)c([2H])c([2H])c23)c([2H])c1[2H]]

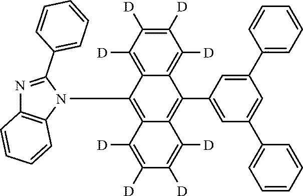 [SMILES: [2H]c1c([2H])c([2H])c2c(-n3c(-c4ccccc4)nc4ccccc43)c3c([2H])c([2H])c([2H])c([2H])c3c(-c3cc(-c4ccccc4)cc(-c4ccccc4)c3)c2c1[2H]]

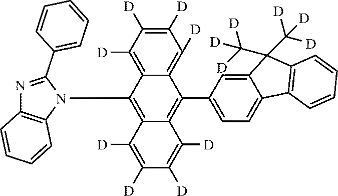 [SMILES: [2H]c1c([2H])c([2H])c2c(-n3c(-c4ccccc4)nc4ccccc43)c3c([2H])c([2H])c([2H])c([2H])c3c(-c3ccc4c(c3)C(C([2H])([2H])[2H])(C([2H])([2H])[2H])c3ccccc3-4)c2c1[2H]]

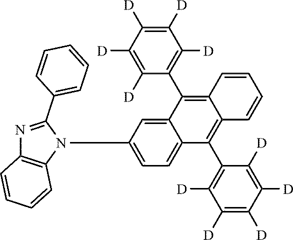 [SMILES: [2H]c1c([2H])c([2H])c(-c2c3ccccc3c(-c3c([2H])c([2H])c([2H])c([2H])c3[2H])c3cc(-n4c(-c5ccccc5)nc5ccccc54)ccc23)c([2H])c1[2H]]

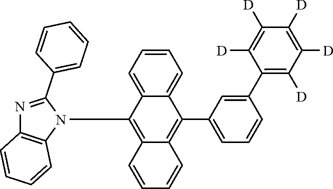 [SMILES: [2H]c1c([2H])c([2H])c(-c2cccc(-c3c4ccccc4c(-n4c(-c5ccccc5)nc5ccccc54)c4ccccc34)c2)c([2H])c1[2H]]

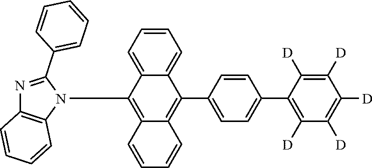 [SMILES: [2H]c1c([2H])c([2H])c(-c2ccc(-c3c4ccccc4c(-n4c(-c5ccccc5)nc5ccccc54)c4ccccc34)cc2)c([2H])c1[2H]]

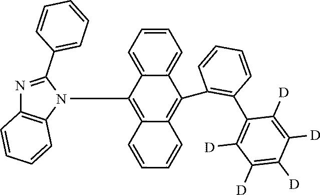 [SMILES: [2H]c1c([2H])c([2H])c(-c2ccccc2-c2c3ccccc3c(-n3c(-c4ccccc4)nc4ccccc43)c3ccccc23)c([2H])c1[2H]]

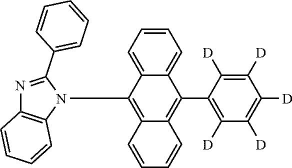 [SMILES: [2H]c1c([2H])c([2H])c(-c2c3ccccc3c(-n3c(-c4ccccc4)nc4ccccc43)c3ccccc23)c([2H])c1[2H]]

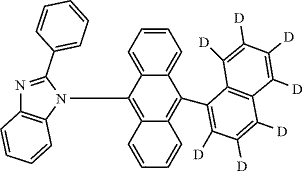 [SMILES: [2H]c1c([2H])c([2H])c2c(-c3c4ccccc4c(-n4c(-c5ccccc5)nc5ccccc54)c4ccccc34)c([2H])c([2H])c([2H])c2c1[2H]]

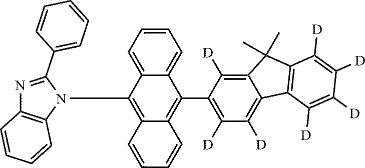 [SMILES: [2H]c1c([2H])c([2H])c2c(c1[2H])-c1c([2H])c([2H])c(-c3c4ccccc4c(-n4c(-c5ccccc5)nc5ccccc54)c4ccccc34)c([2H])c1C2(C)C]

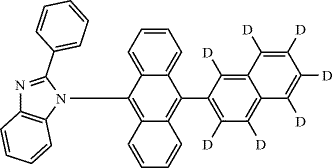 [SMILES: [2H]c1c([2H])c([2H])c2c([2H])c(-c3c4ccccc4c(-n4c(-c5ccccc5)nc5ccccc54)c4ccccc34)c([2H])c([2H])c2c1[2H]]

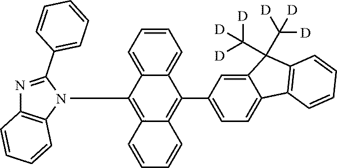 [SMILES: [2H]C([2H])([2H])C1(C([2H])([2H])[2H])c2ccccc2-c2ccc(-c3c4ccccc4c(-n4c(-c5ccccc5)nc5ccccc54)c4ccccc34)cc21]

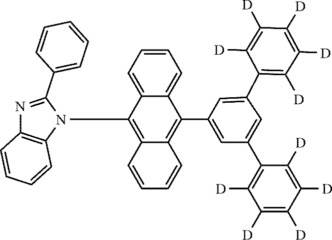 [SMILES: [2H]c1c([2H])c([2H])c(-c2cc(-c3c([2H])c([2H])c([2H])c([2H])c3[2H])cc(-c3c4ccccc4c(-n4c(-c5ccccc5)nc5ccccc54)c4ccccc34)c2)c([2H])c1[2H]]

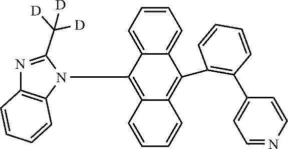 [SMILES: [2H]C([2H])([2H])c1nc2ccccc2n1-c1c2ccccc2c(-c2ccccc2-c2ccncc2)c2ccccc12]

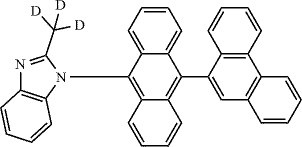 [SMILES: [2H]C([2H])([2H])c1nc2ccccc2n1-c1c2ccccc2c(-c2cc3ccccc3c3ccccc23)c2ccccc12]

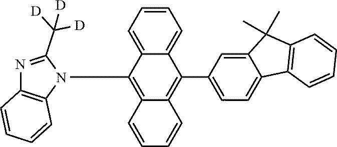 [SMILES: [2H]C([2H])([2H])c1nc2ccccc2n1-c1c2ccccc2c(-c2ccc3c(c2)C(C)(C)c2ccccc2-3)c2ccccc12]

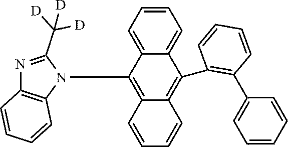 [SMILES: [2H]C([2H])([2H])c1nc2ccccc2n1-c1c2ccccc2c(-c2ccccc2-c2ccccc2)c2ccccc12]

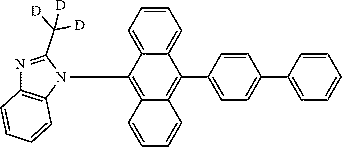 [SMILES: [2H]C([2H])([2H])c1nc2ccccc2n1-c1c2ccccc2c(-c2ccc(-c3ccccc3)cc2)c2ccccc12]

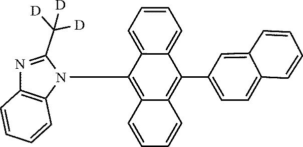 [SMILES: [2H]C([2H])([2H])c1nc2ccccc2n1-c1c2ccccc2c(-c2ccc3ccccc3c2)c2ccccc12]

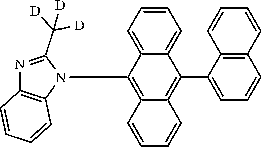 [SMILES: [2H]C([2H])([2H])c1nc2ccccc2n1-c1c2ccccc2c(-c2cccc3ccccc23)c2ccccc12]

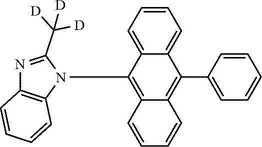 [SMILES: [2H]C([2H])([2H])c1nc2ccccc2n1-c1c2ccccc2c(-c2ccccc2)c2ccccc12]

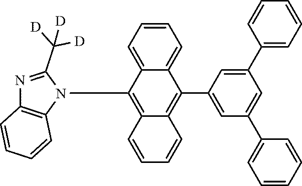 [SMILES: [2H]C([2H])([2H])c1nc2ccccc2n1-c1c2ccccc2c(-c2cc(-c3ccccc3)cc(-c3ccccc3)c2)c2ccccc12]

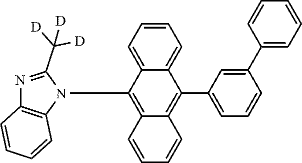 [SMILES: [2H]C([2H])([2H])c1nc2ccccc2n1-c1c2ccccc2c(-c2cccc(-c3ccccc3)c2)c2ccccc12]

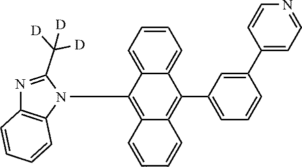 [SMILES: [2H]C([2H])([2H])c1nc2ccccc2n1-c1c2ccccc2c(-c2cccc(-c3ccncc3)c2)c2ccccc12]

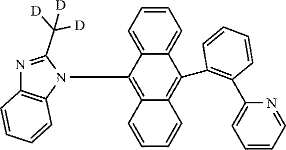 [SMILES: [2H]C([2H])([2H])c1nc2ccccc2n1-c1c2ccccc2c(-c2ccccc2-c2ccccn2)c2ccccc12]

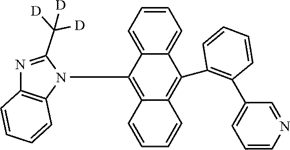 [SMILES: [2H]C([2H])([2H])c1nc2ccccc2n1-c1c2ccccc2c(-c2ccccc2-c2cccnc2)c2ccccc12]

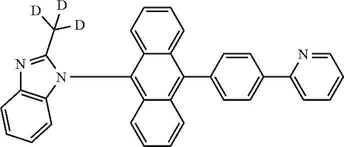 [SMILES: [2H]C([2H])([2H])c1nc2ccccc2n1-c1c2ccccc2c(-c2ccc(-c3ccccn3)cc2)c2ccccc12]

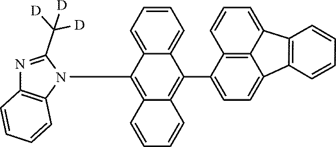 [SMILES: [2H]C([2H])([2H])c1nc2ccccc2n1-c1c2ccccc2c(-c2ccc3c4c(cccc24)-c2ccccc2-3)c2ccccc12]

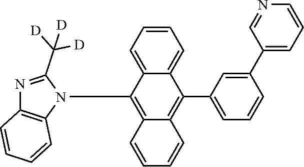 [SMILES: [2H]C([2H])([2H])c1nc2ccccc2n1-c1c2ccccc2c(-c2cccc(-c3cccnc3)c2)c2ccccc12]

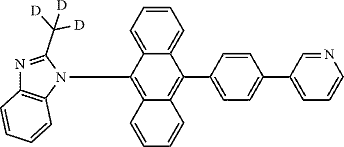 [SMILES: [2H]C([2H])([2H])c1nc2ccccc2n1-c1c2ccccc2c(-c2ccc(-c3cccnc3)cc2)c2ccccc12]

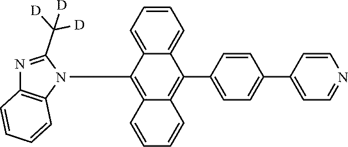 [SMILES: [2H]C([2H])([2H])c1nc2ccccc2n1-c1c2ccccc2c(-c2ccc(-c3ccncc3)cc2)c2ccccc12]

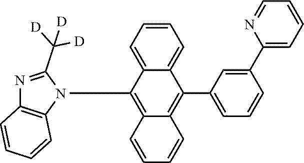 [SMILES: [2H]C([2H])([2H])c1nc2ccccc2n1-c1c2ccccc2c(-c2cccc(-c3ccccn3)c2)c2ccccc12]

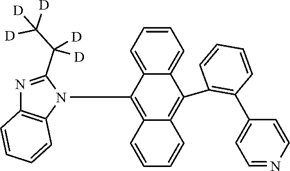 [SMILES: [2H]C([2H])([2H])C([2H])([2H])c1nc2ccccc2n1-c1c2ccccc2c(-c2ccccc2-c2ccncc2)c2ccccc12]

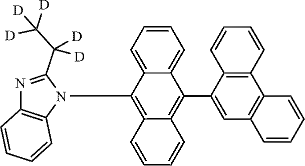 [SMILES: [2H]C([2H])([2H])C([2H])([2H])c1nc2ccccc2n1-c1c2ccccc2c(-c2cc3ccccc3c3ccccc23)c2ccccc12]

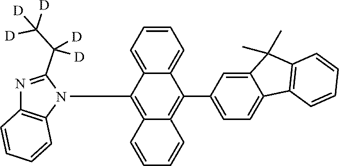 [SMILES: [2H]C([2H])([2H])C([2H])([2H])c1nc2ccccc2n1-c1c2ccccc2c(-c2ccc3c(c2)C(C)(C)c2ccccc2-3)c2ccccc12]

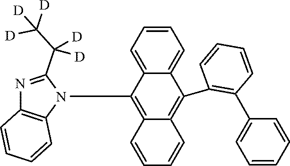 [SMILES: [2H]C([2H])([2H])C([2H])([2H])c1nc2ccccc2n1-c1c2ccccc2c(-c2ccccc2-c2ccccc2)c2ccccc12]

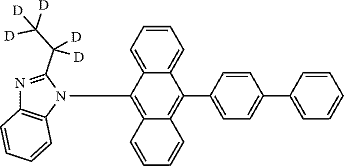 [SMILES: [2H]C([2H])([2H])C([2H])([2H])c1nc2ccccc2n1-c1c2ccccc2c(-c2ccc(-c3ccccc3)cc2)c2ccccc12]

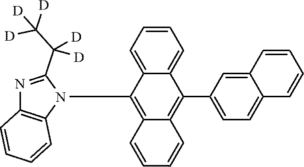 [SMILES: [2H]C([2H])([2H])C([2H])([2H])c1nc2ccccc2n1-c1c2ccccc2c(-c2ccc3ccccc3c2)c2ccccc12]

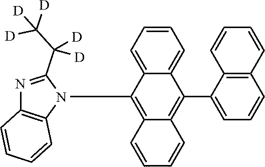 [SMILES: [2H]C([2H])([2H])C([2H])([2H])c1nc2ccccc2n1-c1c2ccccc2c(-c2cccc3ccccc23)c2ccccc12]

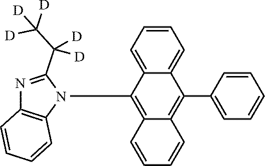 [SMILES: [2H]C([2H])([2H])C([2H])([2H])c1nc2ccccc2n1-c1c2ccccc2c(-c2ccccc2)c2ccccc12]

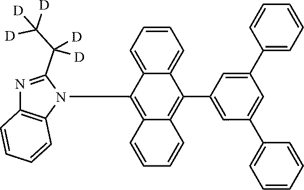 [SMILES: [2H]C([2H])([2H])C([2H])([2H])c1nc2ccccc2n1-c1c2ccccc2c(-c2cc(-c3ccccc3)cc(-c3ccccc3)c2)c2ccccc12]

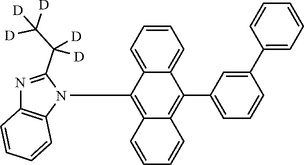 [SMILES: [2H]C([2H])([2H])C([2H])([2H])c1nc2ccccc2n1-c1c2ccccc2c(-c2cccc(-c3ccccc3)c2)c2ccccc12]

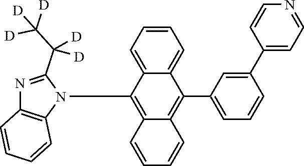 [SMILES: [2H]C([2H])([2H])C([2H])([2H])c1nc2ccccc2n1-c1c2ccccc2c(-c2cccc(-c3ccncc3)c2)c2ccccc12]

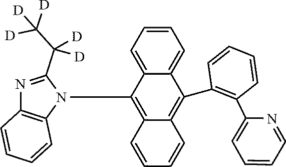 [SMILES: [2H]C([2H])([2H])C([2H])([2H])c1nc2ccccc2n1-c1c2ccccc2c(-c2ccccc2-c2ccccn2)c2ccccc12]

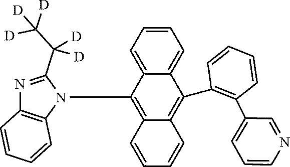 [SMILES: [2H]C([2H])([2H])C([2H])([2H])c1nc2ccccc2n1-c1c2ccccc2c(-c2ccccc2-c2cccnc2)c2ccccc12]

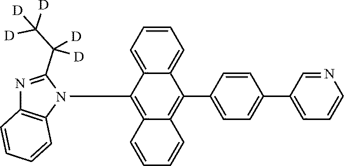 [SMILES: [2H]C([2H])([2H])C([2H])([2H])c1nc2ccccc2n1-c1c2ccccc2c(-c2ccc(-c3cccnc3)cc2)c2ccccc12]

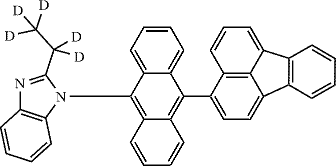 [SMILES: [2H]C([2H])([2H])C([2H])([2H])c1nc2ccccc2n1-c1c2ccccc2c(-c2ccc3c4c(cccc24)-c2ccccc2-3)c2ccccc12]

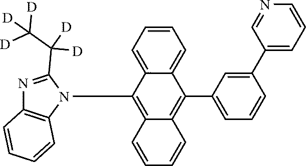 [SMILES: [2H]C([2H])([2H])C([2H])([2H])c1nc2ccccc2n1-c1c2ccccc2c(-c2cccc(-c3cccnc3)c2)c2ccccc12]

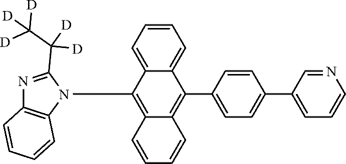 [SMILES: [2H]C([2H])([2H])C([2H])([2H])c1nc2ccccc2n1-c1c2ccccc2c(-c2ccc(-c3cccnc3)cc2)c2ccccc12]

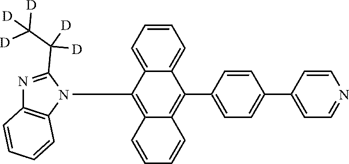 [SMILES: [2H]C([2H])([2H])C([2H])([2H])c1nc2ccccc2n1-c1c2ccccc2c(-c2ccc(-c3ccncc3)cc2)c2ccccc12]

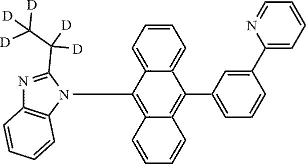 [SMILES: [2H]C([2H])([2H])C([2H])([2H])c1nc2ccccc2n1-c1c2ccccc2c(-c2cccc(-c3ccccn3)c2)c2ccccc12]

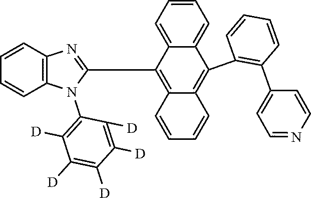 [SMILES: [2H]c1c([2H])c([2H])c(-n2c(-c3c4ccccc4c(-c4ccccc4-c4ccncc4)c4ccccc34)nc3ccccc32)c([2H])c1[2H]]

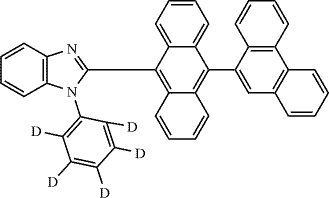 [SMILES: [2H]c1c([2H])c([2H])c(-n2c(-c3c4ccccc4c(-c4cc5ccccc5c5ccccc45)c4ccccc34)nc3ccccc32)c([2H])c1[2H]]

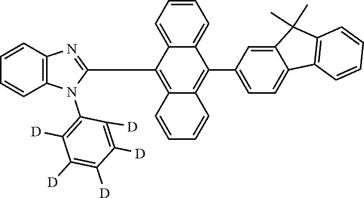 [SMILES: [2H]c1c([2H])c([2H])c(-n2c(-c3c4ccccc4c(-c4ccc5c(c4)C(C)(C)c4ccccc4-5)c4ccccc34)nc3ccccc32)c([2H])c1[2H]]

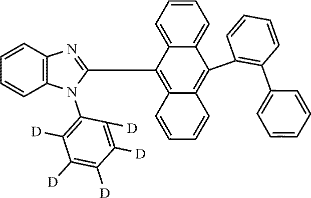 [SMILES: [2H]c1c([2H])c([2H])c(-n2c(-c3c4ccccc4c(-c4ccccc4-c4ccccc4)c4ccccc34)nc3ccccc32)c([2H])c1[2H]]

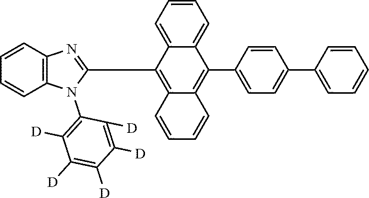 [SMILES: [2H]c1c([2H])c([2H])c(-n2c(-c3c4ccccc4c(-c4ccc(-c5ccccc5)cc4)c4ccccc34)nc3ccccc32)c([2H])c1[2H]]

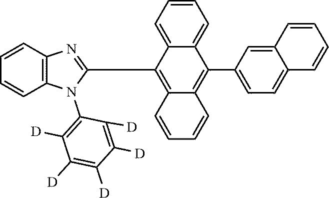 [SMILES: [2H]c1c([2H])c([2H])c(-n2c(-c3c4ccccc4c(-c4ccc5ccccc5c4)c4ccccc34)nc3ccccc32)c([2H])c1[2H]]

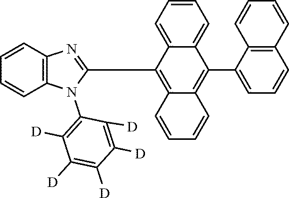 [SMILES: [2H]c1c([2H])c([2H])c(-n2c(-c3c4ccccc4c(-c4cccc5ccccc45)c4ccccc34)nc3ccccc32)c([2H])c1[2H]]

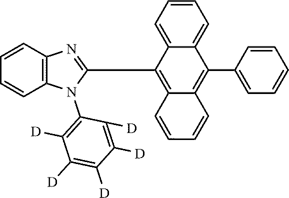 [SMILES: [2H]c1c([2H])c([2H])c(-n2c(-c3c4ccccc4c(-c4ccccc4)c4ccccc34)nc3ccccc32)c([2H])c1[2H]]

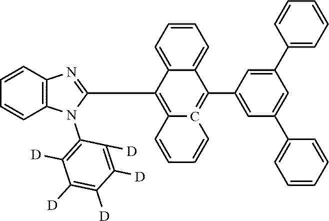 [SMILES: [2H]c1c([2H])c([2H])c(-n2c(-c3c4ccccc4c(-c4cc(-c5ccccc5)cc(-c5ccccc5)c4)c4ccccc34)nc3ccccc32)c([2H])c1[2H]]

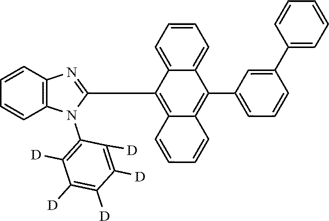 [SMILES: [2H]c1c([2H])c([2H])c(-n2c(-c3c4ccccc4c(-c4cccc(-c5ccccc5)c4)c4ccccc34)nc3ccccc32)c([2H])c1[2H]]

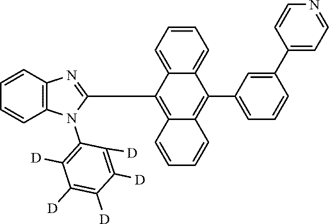 [SMILES: [2H]c1c([2H])c([2H])c(-n2c(-c3c4ccccc4c(-c4cccc(-c5ccncc5)c4)c4ccccc34)nc3ccccc32)c([2H])c1[2H]]

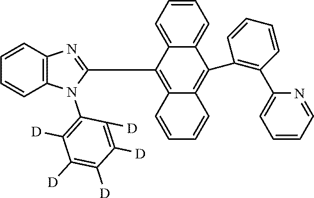 [SMILES: [2H]c1c([2H])c([2H])c(-n2c(-c3c4ccccc4c(-c4ccccc4-c4ccccn4)c4ccccc34)nc3ccccc32)c([2H])c1[2H]]

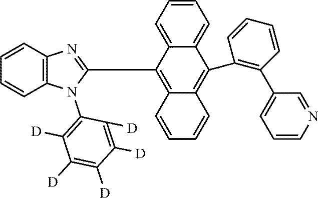 [SMILES: [2H]c1c([2H])c([2H])c(-n2c(-c3c4ccccc4c(-c4ccccc4-c4cccnc4)c4ccccc34)nc3ccccc32)c([2H])c1[2H]]

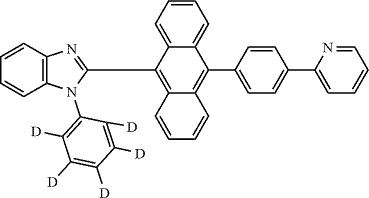 [SMILES: [2H]c1c([2H])c([2H])c(-n2c(-c3c4ccccc4c(-c4ccc(-c5ccccn5)cc4)c4ccccc34)nc3ccccc32)c([2H])c1[2H]]

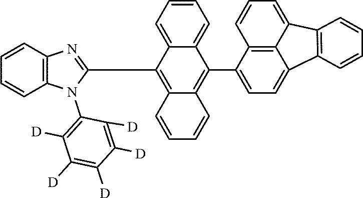 [SMILES: [2H]c1c([2H])c([2H])c(-n2c(-c3c4ccccc4c(-c4ccc5c6c(cccc46)-c4ccccc4-5)c4ccccc34)nc3ccccc32)c([2H])c1[2H]]

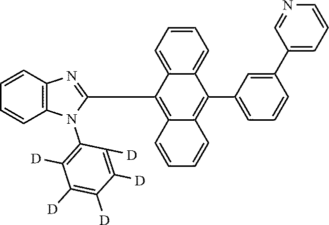 [SMILES: [2H]c1c([2H])c([2H])c(-n2c(-c3c4ccccc4c(-c4cccc(-c5cccnc5)c4)c4ccccc34)nc3ccccc32)c([2H])c1[2H]]

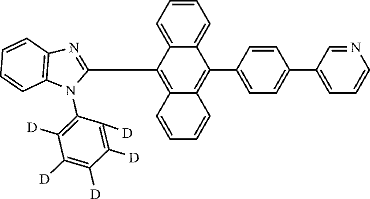 [SMILES: [2H]c1c([2H])c([2H])c(-n2c(-c3c4ccccc4c(-c4ccc(-c5cccnc5)cc4)c4ccccc34)nc3ccccc32)c([2H])c1[2H]]

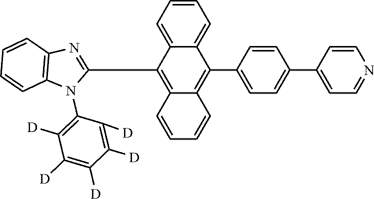 [SMILES: [2H]c1c([2H])c([2H])c(-n2c(-c3c4ccccc4c(-c4ccc(-c5ccncc5)cc4)c4ccccc34)nc3ccccc32)c([2H])c1[2H]]

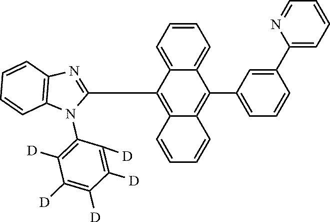 [SMILES: [2H]c1c([2H])c([2H])c(-n2c(-c3c4ccccc4c(-c4cccc(-c5ccccn5)c4)c4ccccc34)nc3ccccc32)c([2H])c1[2H]]

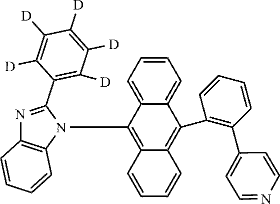 [SMILES: [2H]c1c([2H])c([2H])c(-c2nc3ccccc3n2-c2c3ccccc3c(-c3ccccc3-c3ccncc3)c3ccccc23)c([2H])c1[2H]]

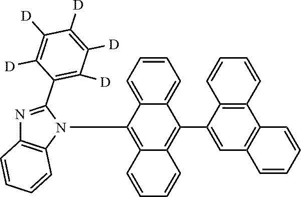 [SMILES: [2H]c1c([2H])c([2H])c(-c2nc3ccccc3n2-c2c3ccccc3c(-c3cc4ccccc4c4ccccc34)c3ccccc23)c([2H])c1[2H]]

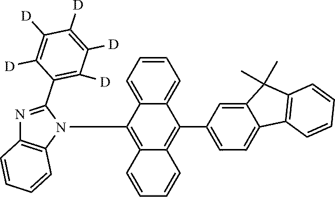 [SMILES: [2H]c1c([2H])c([2H])c(-c2nc3ccccc3n2-c2c3ccccc3c(-c3ccc4c(c3)C(C)(C)c3ccccc3-4)c3ccccc23)c([2H])c1[2H]]

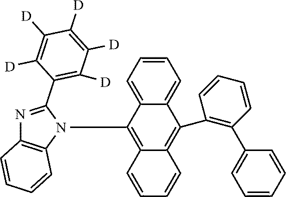 [SMILES: [2H]c1c([2H])c([2H])c(-c2nc3ccccc3n2-c2c3ccccc3c(-c3ccccc3-c3ccccc3)c3ccccc23)c([2H])c1[2H]]

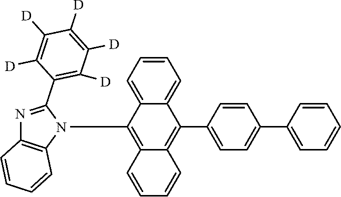 [SMILES: [2H]c1c([2H])c([2H])c(-c2nc3ccccc3n2-c2c3ccccc3c(-c3ccc(-c4ccccc4)cc3)c3ccccc23)c([2H])c1[2H]]

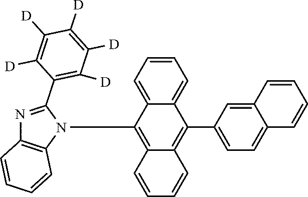 [SMILES: [2H]c1c([2H])c([2H])c(-c2nc3ccccc3n2-c2c3ccccc3c(-c3ccc4ccccc4c3)c3ccccc23)c([2H])c1[2H]]

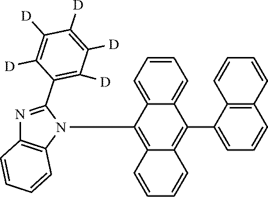 [SMILES: [2H]c1c([2H])c([2H])c(-c2nc3ccccc3n2-c2c3ccccc3c(-c3cccc4ccccc34)c3ccccc23)c([2H])c1[2H]]

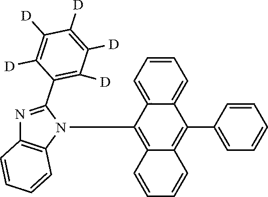 [SMILES: [2H]c1c([2H])c([2H])c(-c2nc3ccccc3n2-c2c3ccccc3c(-c3ccccc3)c3ccccc23)c([2H])c1[2H]]

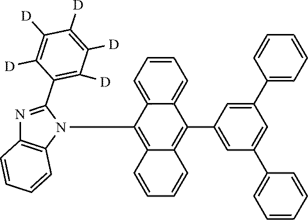 [SMILES: [2H]c1c([2H])c([2H])c(-c2nc3ccccc3n2-c2c3ccccc3c(-c3cc(-c4ccccc4)cc(-c4ccccc4)c3)c3ccccc23)c([2H])c1[2H]]

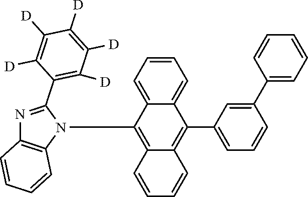 [SMILES: [2H]c1c([2H])c([2H])c(-c2nc3ccccc3n2-c2c3ccccc3c(-c3cccc(-c4ccccc4)c3)c3ccccc23)c([2H])c1[2H]]

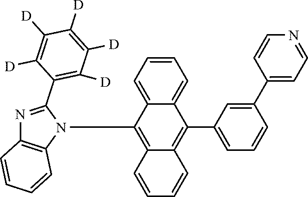 [SMILES: [2H]c1c([2H])c([2H])c(-c2nc3ccccc3n2-c2c3ccccc3c(-c3cccc(-c4ccncc4)c3)c3ccccc23)c([2H])c1[2H]]

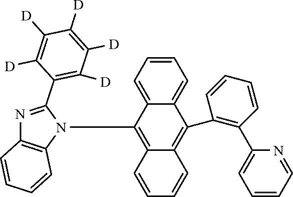 [SMILES: [2H]c1c([2H])c([2H])c(-c2nc3ccccc3n2-c2c3ccccc3c(-c3ccccc3-c3ccccn3)c3ccccc23)c([2H])c1[2H]]

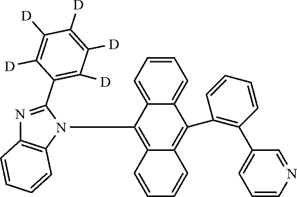 [SMILES: [2H]c1c([2H])c([2H])c(-c2nc3ccccc3n2-c2c3ccccc3c(-c3ccccc3-c3cccnc3)c3ccccc23)c([2H])c1[2H]]

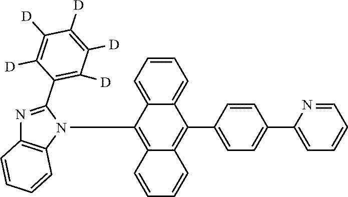 [SMILES: [2H]c1c([2H])c([2H])c(-c2nc3ccccc3n2-c2c3ccccc3c(-c3ccc(-c4ccccn4)cc3)c3ccccc23)c([2H])c1[2H]]

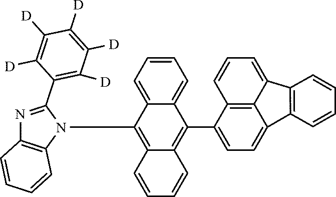 [SMILES: [2H]c1c([2H])c([2H])c(-c2nc3ccccc3n2-c2c3ccccc3c(-c3ccc4c5c(cccc35)-c3ccccc3-4)c3ccccc23)c([2H])c1[2H]]

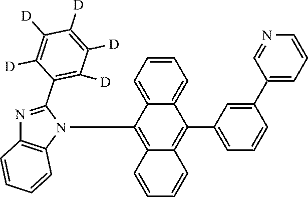 [SMILES: [2H]c1c([2H])c([2H])c(-c2nc3ccccc3n2-c2c3ccccc3c(-c3cccc(-c4cccnc4)c3)c3ccccc23)c([2H])c1[2H]]

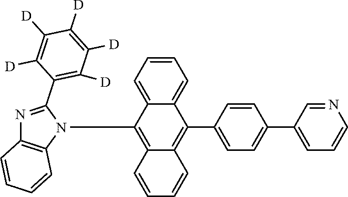 [SMILES: [2H]c1c([2H])c([2H])c(-c2nc3ccccc3n2-c2c3ccccc3c(-c3ccc(-c4cccnc4)cc3)c3ccccc23)c([2H])c1[2H]]

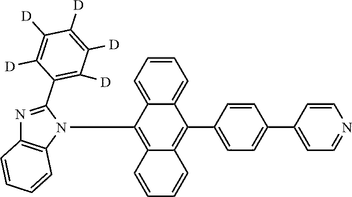 [SMILES: [2H]c1c([2H])c([2H])c(-c2nc3ccccc3n2-c2c3ccccc3c(-c3ccc(-c4ccncc4)cc3)c3ccccc23)c([2H])c1[2H]]

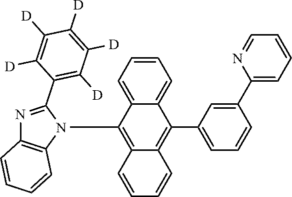 [SMILES: [2H]c1c([2H])c([2H])c(-c2nc3ccccc3n2-c2c3ccccc3c(-c3cccc(-c4ccccn4)c3)c3ccccc23)c([2H])c1[2H]]

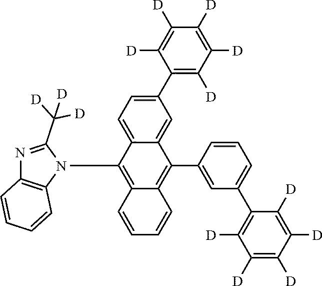 [SMILES: [2H]c1c([2H])c([2H])c(-c2cccc(-c3c4ccccc4c(-n4c(C([2H])([2H])[2H])nc5ccccc54)c4ccc(-c5c([2H])c([2H])c([2H])c([2H])c5[2H])cc34)c2)c([2H])c1[2H]]

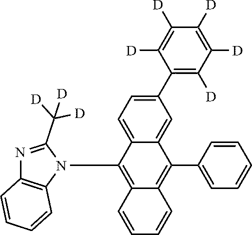 [SMILES: [2H]c1c([2H])c([2H])c(-c2ccc3c(-n4c(C([2H])([2H])[2H])nc5ccccc54)c4ccccc4c(-c4ccccc4)c3c2)c([2H])c1[2H]]

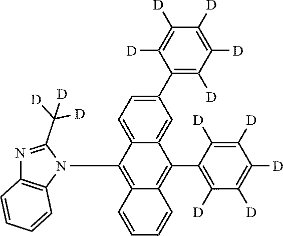 [SMILES: [2H]c1c([2H])c([2H])c(-c2ccc3c(-n4c(C([2H])([2H])[2H])nc5ccccc54)c4ccccc4c(-c4c([2H])c([2H])c([2H])c([2H])c4[2H])c3c2)c([2H])c1[2H]]

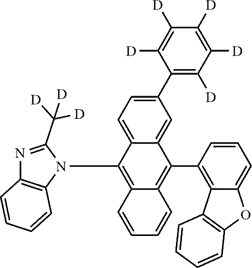 [SMILES: [2H]c1c([2H])c([2H])c(-c2ccc3c(-n4c(C([2H])([2H])[2H])nc5ccccc54)c4ccccc4c(-c4cccc5oc6ccccc6c45)c3c2)c([2H])c1[2H]]

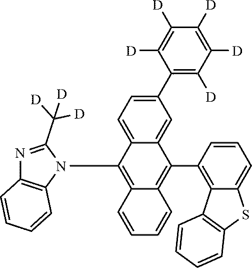 [SMILES: [2H]c1c([2H])c([2H])c(-c2ccc3c(-n4c(C([2H])([2H])[2H])nc5ccccc54)c4ccccc4c(-c4cccc5sc6ccccc6c45)c3c2)c([2H])c1[2H]]

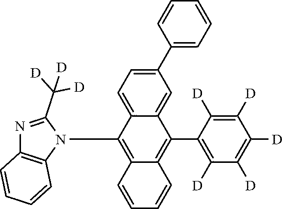 [SMILES: [2H]c1c([2H])c([2H])c(-c2c3ccccc3c(-n3c(C([2H])([2H])[2H])nc4ccccc43)c3ccc(-c4ccccc4)cc23)c([2H])c1[2H]]

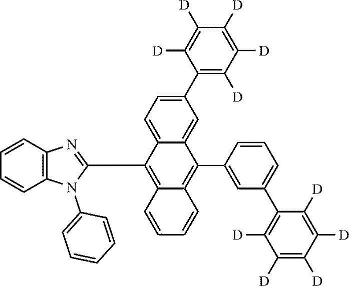 [SMILES: [2H]c1c([2H])c([2H])c(-c2cccc(-c3c4ccccc4c(-c4nc5ccccc5n4-c4ccccc4)c4ccc(-c5c([2H])c([2H])c([2H])c([2H])c5[2H])cc34)c2)c([2H])c1[2H]]

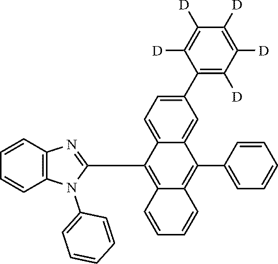 [SMILES: [2H]c1c([2H])c([2H])c(-c2ccc3c(-c4nc5ccccc5n4-c4ccccc4)c4ccccc4c(-c4ccccc4)c3c2)c([2H])c1[2H]]

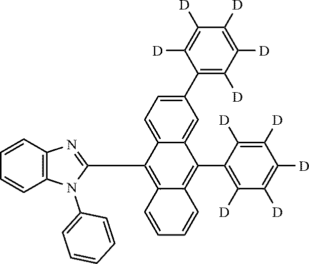 [SMILES: [2H]c1c([2H])c([2H])c(-c2ccc3c(-c4nc5ccccc5n4-c4ccccc4)c4ccccc4c(-c4c([2H])c([2H])c([2H])c([2H])c4[2H])c3c2)c([2H])c1[2H]]

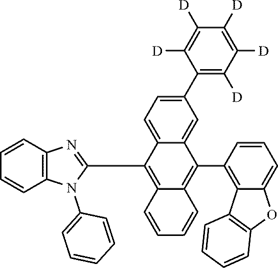 [SMILES: [2H]c1c([2H])c([2H])c(-c2ccc3c(-c4nc5ccccc5n4-c4ccccc4)c4ccccc4c(-c4cccc5oc6ccccc6c45)c3c2)c([2H])c1[2H]]

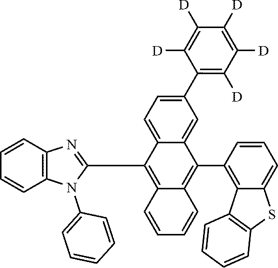 [SMILES: [2H]c1c([2H])c([2H])c(-c2ccc3c(-c4nc5ccccc5n4-c4ccccc4)c4ccccc4c(-c4cccc5sc6ccccc6c45)c3c2)c([2H])c1[2H]]

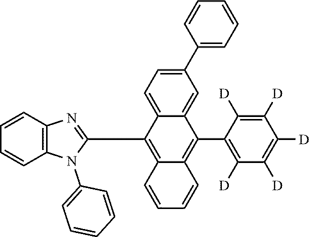 [SMILES: [2H]c1c([2H])c([2H])c(-c2c3ccccc3c(-c3nc4ccccc4n3-c3ccccc3)c3ccc(-c4ccccc4)cc23)c([2H])c1[2H]]

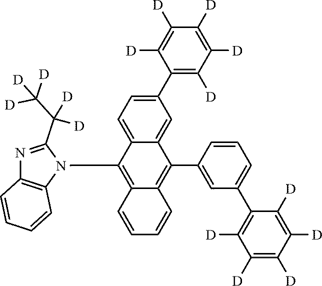 [SMILES: [2H]c1c([2H])c([2H])c(-c2cccc(-c3c4ccccc4c(-n4c(C([2H])([2H])C([2H])([2H])[2H])nc5ccccc54)c4ccc(-c5c([2H])c([2H])c([2H])c([2H])c5[2H])cc34)c2)c([2H])c1[2H]]

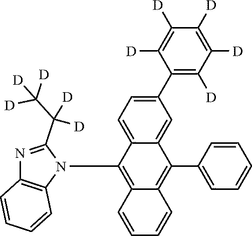 [SMILES: [2H]c1c([2H])c([2H])c(-c2ccc3c(-n4c(C([2H])([2H])C([2H])([2H])[2H])nc5ccccc54)c4ccccc4c(-c4ccccc4)c3c2)c([2H])c1[2H]]

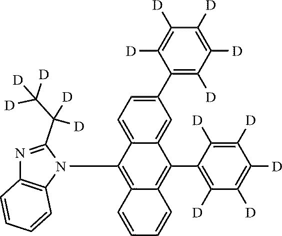 [SMILES: [2H]c1c([2H])c([2H])c(-c2ccc3c(-n4c(C([2H])([2H])C([2H])([2H])[2H])nc5ccccc54)c4ccccc4c(-c4c([2H])c([2H])c([2H])c([2H])c4[2H])c3c2)c([2H])c1[2H]]

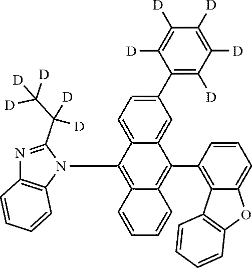 [SMILES: [2H]c1c([2H])c([2H])c(-c2ccc3c(-n4c(C([2H])([2H])C([2H])([2H])[2H])nc5ccccc54)c4ccccc4c(-c4cccc5oc6ccccc6c45)c3c2)c([2H])c1[2H]]

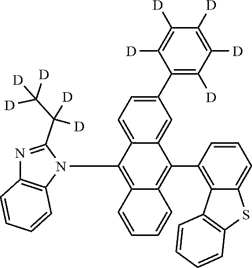 [SMILES: [2H]c1c([2H])c([2H])c(-c2ccc3c(-n4c(C([2H])([2H])C([2H])([2H])[2H])nc5ccccc54)c4ccccc4c(-c4cccc5sc6ccccc6c45)c3c2)c([2H])c1[2H]]

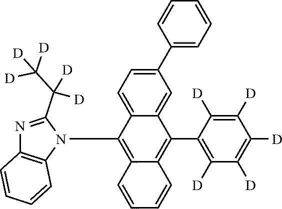 [SMILES: [2H]c1c([2H])c([2H])c(-c2c3ccccc3c(-n3c(C([2H])([2H])C([2H])([2H])[2H])nc4ccccc43)c3ccc(-c4ccccc4)cc23)c([2H])c1[2H]]

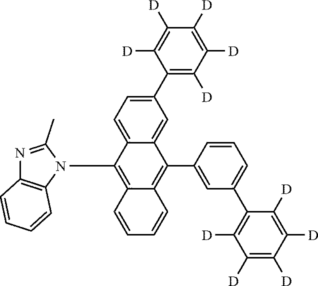 [SMILES: [2H]c1c([2H])c([2H])c(-c2cccc(-c3c4ccccc4c(-n4c(C)nc5ccccc54)c4ccc(-c5c([2H])c([2H])c([2H])c([2H])c5[2H])cc34)c2)c([2H])c1[2H]]

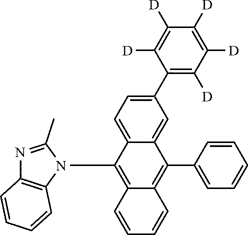 [SMILES: [2H]c1c([2H])c([2H])c(-c2ccc3c(-n4c(C)nc5ccccc54)c4ccccc4c(-c4ccccc4)c3c2)c([2H])c1[2H]]

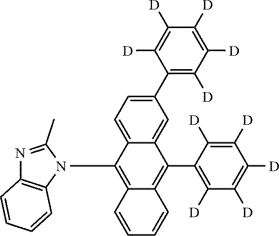 [SMILES: [2H]c1c([2H])c([2H])c(-c2ccc3c(-n4c(C)nc5ccccc54)c4ccccc4c(-c4c([2H])c([2H])c([2H])c([2H])c4[2H])c3c2)c([2H])c1[2H]]

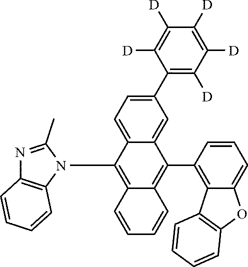 [SMILES: [2H]c1c([2H])c([2H])c(-c2ccc3c(-n4c(C)nc5ccccc54)c4ccccc4c(-c4cccc5oc6ccccc6c45)c3c2)c([2H])c1[2H]]

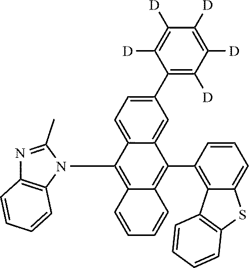 [SMILES: [2H]c1c([2H])c([2H])c(-c2ccc3c(-n4c(C)nc5ccccc54)c4ccccc4c(-c4cccc5sc6ccccc6c45)c3c2)c([2H])c1[2H]]

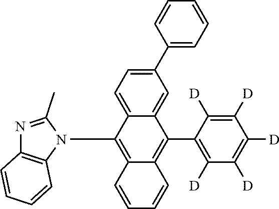 [SMILES: [2H]c1c([2H])c([2H])c(-c2c3ccccc3c(-n3c(C)nc4ccccc43)c3ccc(-c4ccccc4)cc23)c([2H])c1[2H]]

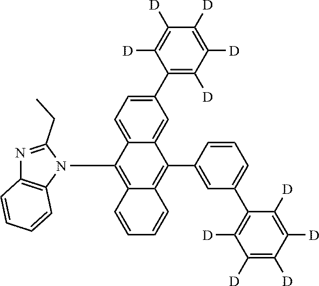 [SMILES: [2H]c1c([2H])c([2H])c(-c2cccc(-c3c4ccccc4c(-n4c(CC)nc5ccccc54)c4ccc(-c5c([2H])c([2H])c([2H])c([2H])c5[2H])cc34)c2)c([2H])c1[2H]]

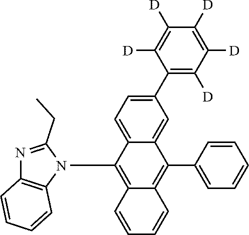 [SMILES: [2H]c1c([2H])c([2H])c(-c2ccc3c(-n4c(CC)nc5ccccc54)c4ccccc4c(-c4ccccc4)c3c2)c([2H])c1[2H]]

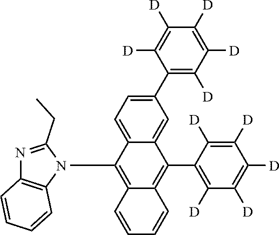 [SMILES: [2H]c1c([2H])c([2H])c(-c2ccc3c(-n4c(CC)nc5ccccc54)c4ccccc4c(-c4c([2H])c([2H])c([2H])c([2H])c4[2H])c3c2)c([2H])c1[2H]]

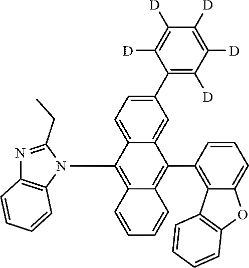 [SMILES: [2H]c1c([2H])c([2H])c(-c2ccc3c(-n4c(CC)nc5ccccc54)c4ccccc4c(-c4cccc5oc6ccccc6c45)c3c2)c([2H])c1[2H]]

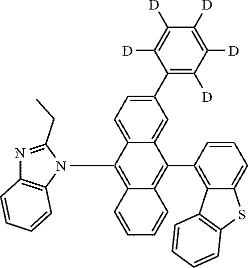 [SMILES: [2H]c1c([2H])c([2H])c(-c2ccc3c(-n4c(CC)nc5ccccc54)c4ccccc4c(-c4cccc5sc6ccccc6c45)c3c2)c([2H])c1[2H]]

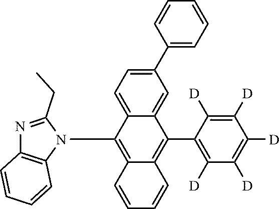 [SMILES: [2H]c1c([2H])c([2H])c(-c2c3ccccc3c(-n3c(CC)nc4ccccc43)c3ccc(-c4ccccc4)cc23)c([2H])c1[2H]]

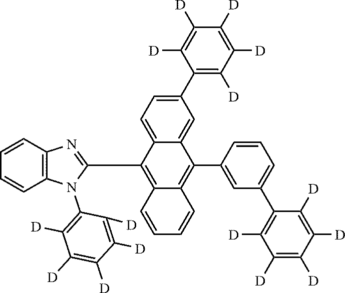 [SMILES: [2H]c1c([2H])c([2H])c(-c2cccc(-c3c4ccccc4c(-c4nc5ccccc5n4-c4c([2H])c([2H])c([2H])c([2H])c4[2H])c4ccc(-c5c([2H])c([2H])c([2H])c([2H])c5[2H])cc34)c2)c([2H])c1[2H]]

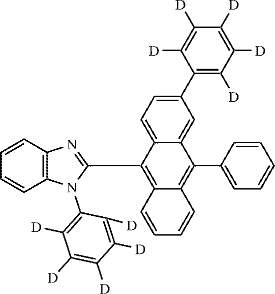 [SMILES: [2H]c1c([2H])c([2H])c(-c2ccc3c(-c4nc5ccccc5n4-c4c([2H])c([2H])c([2H])c([2H])c4[2H])c4ccccc4c(-c4ccccc4)c3c2)c([2H])c1[2H]]

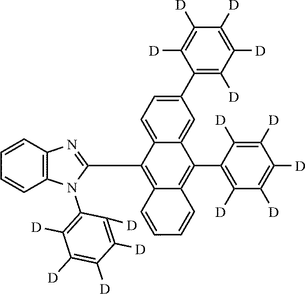 [SMILES: [2H]c1c([2H])c([2H])c(-c2ccc3c(-c4nc5ccccc5n4-c4c([2H])c([2H])c([2H])c([2H])c4[2H])c4ccccc4c(-c4c([2H])c([2H])c([2H])c([2H])c4[2H])c3c2)c([2H])c1[2H]]

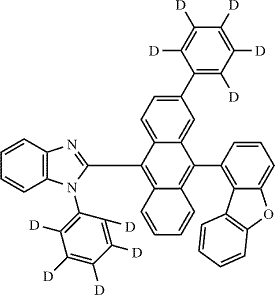 [SMILES: [2H]c1c([2H])c([2H])c(-c2ccc3c(-c4nc5ccccc5n4-c4c([2H])c([2H])c([2H])c([2H])c4[2H])c4ccccc4c(-c4cccc5oc6ccccc6c45)c3c2)c([2H])c1[2H]]

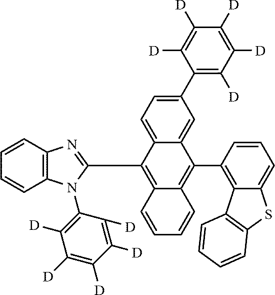 [SMILES: [2H]c1c([2H])c([2H])c(-c2ccc3c(-c4nc5ccccc5n4-c4c([2H])c([2H])c([2H])c([2H])c4[2H])c4ccccc4c(-c4cccc5sc6ccccc6c45)c3c2)c([2H])c1[2H]]

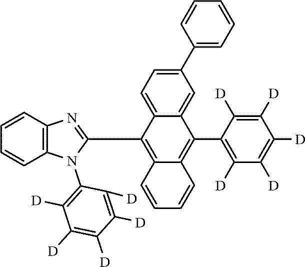 [SMILES: [2H]c1c([2H])c([2H])c(-c2c3ccccc3c(-c3nc4ccccc4n3-c3c([2H])c([2H])c([2H])c([2H])c3[2H])c3ccc(-c4ccccc4)cc23)c([2H])c1[2H]]

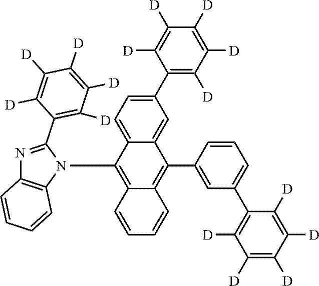 [SMILES: [2H]c1c([2H])c([2H])c(-c2cccc(-c3c4ccccc4c(-n4c(-c5c([2H])c([2H])c([2H])c([2H])c5[2H])nc5ccccc54)c4ccc(-c5c([2H])c([2H])c([2H])c([2H])c5[2H])cc34)c2)c([2H])c1[2H]]

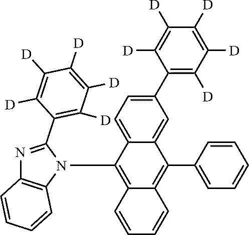 [SMILES: [2H]c1c([2H])c([2H])c(-c2ccc3c(-n4c(-c5c([2H])c([2H])c([2H])c([2H])c5[2H])nc5ccccc54)c4ccccc4c(-c4ccccc4)c3c2)c([2H])c1[2H]]

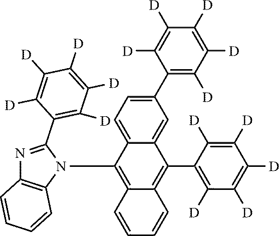 [SMILES: [2H]c1c([2H])c([2H])c(-c2ccc3c(-n4c(-c5c([2H])c([2H])c([2H])c([2H])c5[2H])nc5ccccc54)c4ccccc4c(-c4c([2H])c([2H])c([2H])c([2H])c4[2H])c3c2)c([2H])c1[2H]]

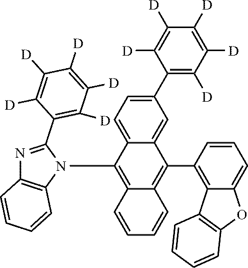 [SMILES: [2H]c1c([2H])c([2H])c(-c2ccc3c(-n4c(-c5c([2H])c([2H])c([2H])c([2H])c5[2H])nc5ccccc54)c4ccccc4c(-c4cccc5oc6ccccc6c45)c3c2)c([2H])c1[2H]]

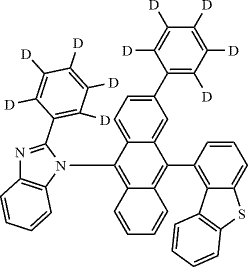 [SMILES: [2H]c1c([2H])c([2H])c(-c2ccc3c(-n4c(-c5c([2H])c([2H])c([2H])c([2H])c5[2H])nc5ccccc54)c4ccccc4c(-c4cccc5sc6ccccc6c45)c3c2)c([2H])c1[2H]]

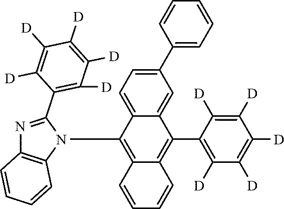 [SMILES: [2H]c1c([2H])c([2H])c(-c2c3ccccc3c(-n3c(-c4c([2H])c([2H])c([2H])c([2H])c4[2H])nc4ccccc43)c3ccc(-c4ccccc4)cc23)c([2H])c1[2H]]

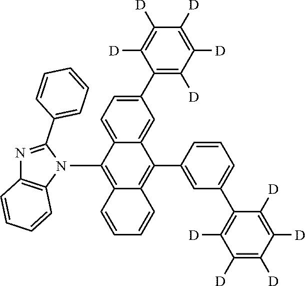 [SMILES: [2H]c1c([2H])c([2H])c(-c2cccc(-c3c4ccccc4c(-n4c(-c5ccccc5)nc5ccccc54)c4ccc(-c5c([2H])c([2H])c([2H])c([2H])c5[2H])cc34)c2)c([2H])c1[2H]]

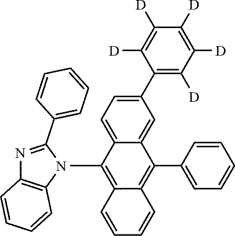 [SMILES: [2H]c1c([2H])c([2H])c(-c2ccc3c(-n4c(-c5ccccc5)nc5ccccc54)c4ccccc4c(-c4ccccc4)c3c2)c([2H])c1[2H]]

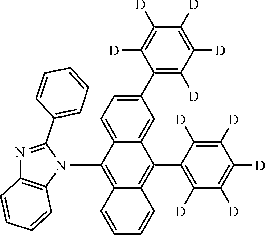 [SMILES: [2H]c1c([2H])c([2H])c(-c2ccc3c(-n4c(-c5ccccc5)nc5ccccc54)c4ccccc4c(-c4c([2H])c([2H])c([2H])c([2H])c4[2H])c3c2)c([2H])c1[2H]]

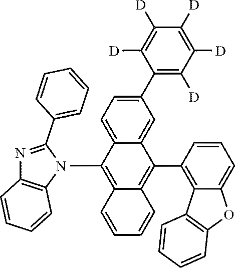 [SMILES: [2H]c1c([2H])c([2H])c(-c2ccc3c(-n4c(-c5ccccc5)nc5ccccc54)c4ccccc4c(-c4cccc5oc6ccccc6c45)c3c2)c([2H])c1[2H]]

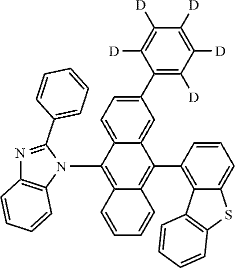 [SMILES: [2H]c1c([2H])c([2H])c(-c2ccc3c(-n4c(-c5ccccc5)nc5ccccc54)c4ccccc4c(-c4cccc5sc6ccccc6c45)c3c2)c([2H])c1[2H]]

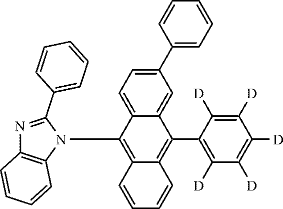 [SMILES: [2H]c1c([2H])c([2H])c(-c2c3ccccc3c(-n3c(-c4ccccc4)nc4ccccc43)c3ccc(-c4ccccc4)cc23)c([2H])c1[2H]]

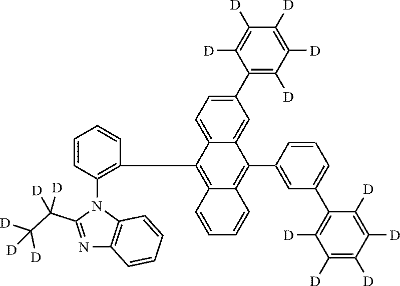 [SMILES: [2H]c1c([2H])c([2H])c(-c2cccc(-c3c4ccccc4c(-c4ccccc4-n4c(C([2H])([2H])C([2H])([2H])[2H])nc5ccccc54)c4ccc(-c5c([2H])c([2H])c([2H])c([2H])c5[2H])cc34)c2)c([2H])c1[2H]]

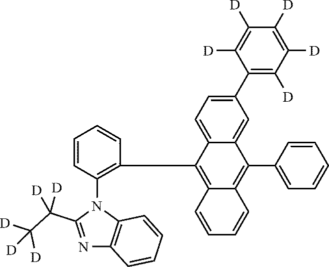 [SMILES: [2H]c1c([2H])c([2H])c(-c2ccc3c(-c4ccccc4-n4c(C([2H])([2H])C([2H])([2H])[2H])nc5ccccc54)c4ccccc4c(-c4ccccc4)c3c2)c([2H])c1[2H]]

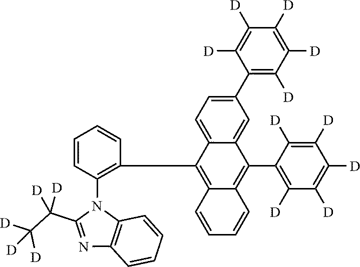 [SMILES: [2H]c1c([2H])c([2H])c(-c2ccc3c(-c4ccccc4-n4c(C([2H])([2H])C([2H])([2H])[2H])nc5ccccc54)c4ccccc4c(-c4c([2H])c([2H])c([2H])c([2H])c4[2H])c3c2)c([2H])c1[2H]]

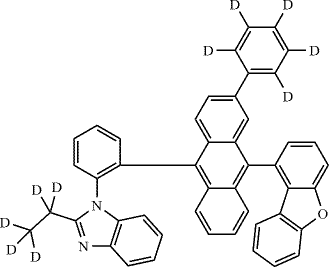 [SMILES: [2H]c1c([2H])c([2H])c(-c2ccc3c(-c4ccccc4-n4c(C([2H])([2H])C([2H])([2H])[2H])nc5ccccc54)c4ccccc4c(-c4cccc5oc6ccccc6c45)c3c2)c([2H])c1[2H]]

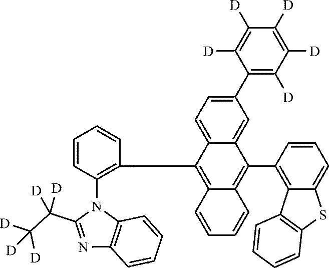 [SMILES: [2H]c1c([2H])c([2H])c(-c2ccc3c(-c4ccccc4-n4c(C([2H])([2H])C([2H])([2H])[2H])nc5ccccc54)c4ccccc4c(-c4cccc5sc6ccccc6c45)c3c2)c([2H])c1[2H]]

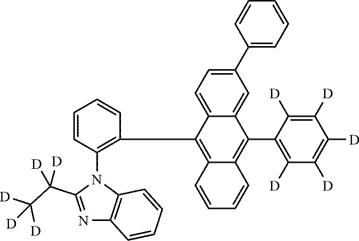 [SMILES: [2H]c1c([2H])c([2H])c(-c2c3ccccc3c(-c3ccccc3-n3c(C([2H])([2H])C([2H])([2H])[2H])nc4ccccc43)c3ccc(-c4ccccc4)cc23)c([2H])c1[2H]]

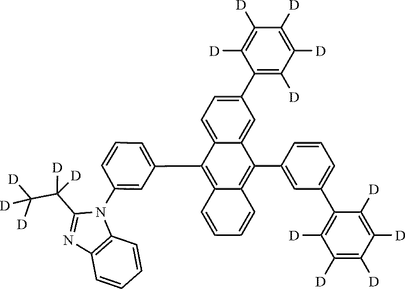 [SMILES: [2H]c1c([2H])c([2H])c(-c2cccc(-c3c4ccccc4c(-c4cccc(-n5c(C([2H])([2H])C([2H])([2H])[2H])nc6ccccc65)c4)c4ccc(-c5c([2H])c([2H])c([2H])c([2H])c5[2H])cc34)c2)c([2H])c1[2H]]

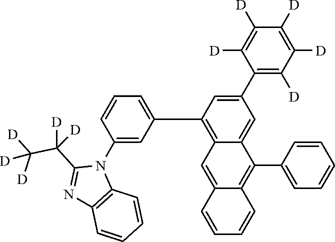 [SMILES: [2H]c1c([2H])c([2H])c(-c2cc(-c3cccc(-n4c(C([2H])([2H])C([2H])([2H])[2H])nc5ccccc54)c3)c3cc4ccccc4c(-c4ccccc4)c3c2)c([2H])c1[2H]]

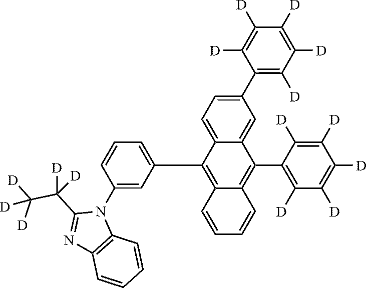 [SMILES: [2H]c1c([2H])c([2H])c(-c2ccc3c(-c4cccc(-n5c(C([2H])([2H])C([2H])([2H])[2H])nc6ccccc65)c4)c4ccccc4c(-c4c([2H])c([2H])c([2H])c([2H])c4[2H])c3c2)c([2H])c1[2H]]

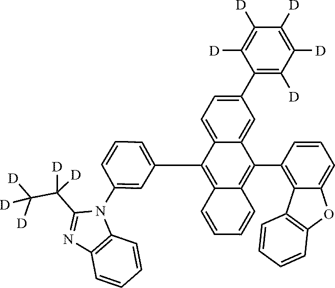 [SMILES: [2H]c1c([2H])c([2H])c(-c2ccc3c(-c4cccc(-n5c(C([2H])([2H])C([2H])([2H])[2H])nc6ccccc65)c4)c4ccccc4c(-c4cccc5oc6ccccc6c45)c3c2)c([2H])c1[2H]]

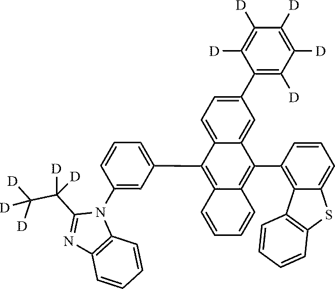 [SMILES: [2H]c1c([2H])c([2H])c(-c2ccc3c(-c4cccc(-n5c(C([2H])([2H])C([2H])([2H])[2H])nc6ccccc65)c4)c4ccccc4c(-c4cccc5sc6ccccc6c45)c3c2)c([2H])c1[2H]]

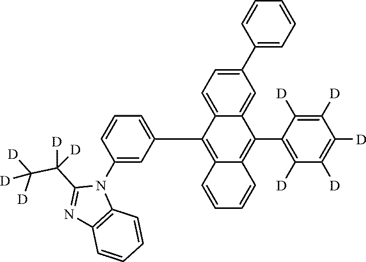 [SMILES: [2H]c1c([2H])c([2H])c(-c2c3ccccc3c(-c3cccc(-n4c(C([2H])([2H])C([2H])([2H])[2H])nc5ccccc54)c3)c3ccc(-c4ccccc4)cc23)c([2H])c1[2H]]

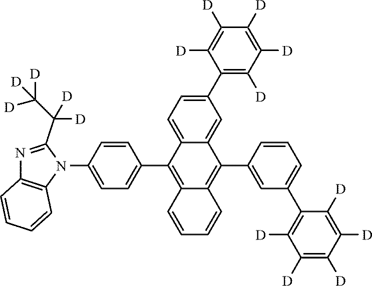 [SMILES: [2H]c1c([2H])c([2H])c(-c2cccc(-c3c4ccccc4c(-c4ccc(-n5c(C([2H])([2H])C([2H])([2H])[2H])nc6ccccc65)cc4)c4ccc(-c5c([2H])c([2H])c([2H])c([2H])c5[2H])cc34)c2)c([2H])c1[2H]]

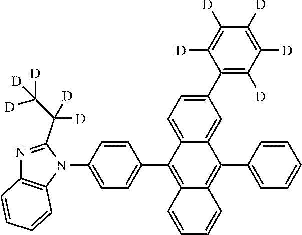 [SMILES: [2H]c1c([2H])c([2H])c(-c2ccc3c(-c4ccc(-n5c(C([2H])([2H])C([2H])([2H])[2H])nc6ccccc65)cc4)c4ccccc4c(-c4ccccc4)c3c2)c([2H])c1[2H]]

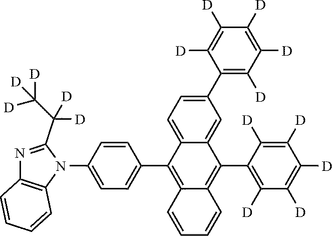 [SMILES: [2H]c1c([2H])c([2H])c(-c2ccc3c(-c4ccc(-n5c(C([2H])([2H])C([2H])([2H])[2H])nc6ccccc65)cc4)c4ccccc4c(-c4c([2H])c([2H])c([2H])c([2H])c4[2H])c3c2)c([2H])c1[2H]]

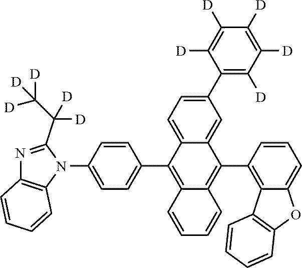 [SMILES: [2H]c1c([2H])c([2H])c(-c2ccc3c(-c4ccc(-n5c(C([2H])([2H])C([2H])([2H])[2H])nc6ccccc65)cc4)c4ccccc4c(-c4cccc5oc6ccccc6c45)c3c2)c([2H])c1[2H]]

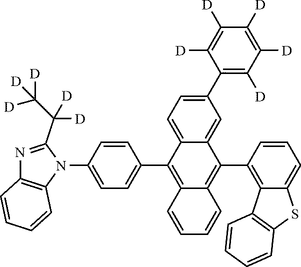 [SMILES: [2H]c1c([2H])c([2H])c(-c2ccc3c(-c4ccc(-n5c(C([2H])([2H])C([2H])([2H])[2H])nc6ccccc65)cc4)c4ccccc4c(-c4cccc5sc6ccccc6c45)c3c2)c([2H])c1[2H]]

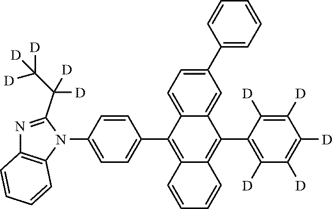 [SMILES: [2H]c1c([2H])c([2H])c(-c2c3ccccc3c(-c3ccc(-n4c(C([2H])([2H])C([2H])([2H])[2H])nc5ccccc54)cc3)c3ccc(-c4ccccc4)cc23)c([2H])c1[2H]]

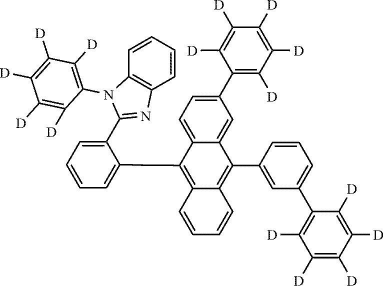 [SMILES: [2H]c1c([2H])c([2H])c(-c2cccc(-c3c4ccccc4c(-c4ccccc4-c4nc5ccccc5n4-c4c([2H])c([2H])c([2H])c([2H])c4[2H])c4ccc(-c5c([2H])c([2H])c([2H])c([2H])c5[2H])cc34)c2)c([2H])c1[2H]]

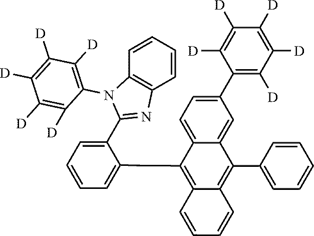 [SMILES: [2H]c1c([2H])c([2H])c(-c2ccc3c(-c4ccccc4-c4nc5ccccc5n4-c4c([2H])c([2H])c([2H])c([2H])c4[2H])c4ccccc4c(-c4ccccc4)c3c2)c([2H])c1[2H]]

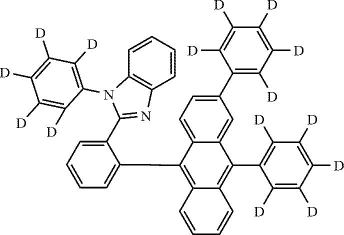 [SMILES: [2H]c1c([2H])c([2H])c(-c2ccc3c(-c4ccccc4-c4nc5ccccc5n4-c4c([2H])c([2H])c([2H])c([2H])c4[2H])c4ccccc4c(-c4c([2H])c([2H])c([2H])c([2H])c4[2H])c3c2)c([2H])c1[2H]]

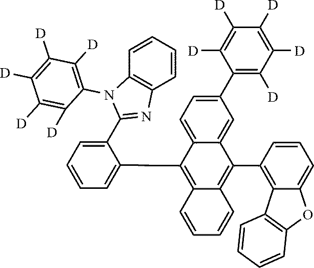 [SMILES: [2H]c1c([2H])c([2H])c(-c2ccc3c(-c4ccccc4-c4nc5ccccc5n4-c4c([2H])c([2H])c([2H])c([2H])c4[2H])c4ccccc4c(-c4cccc5oc6ccccc6c45)c3c2)c([2H])c1[2H]]

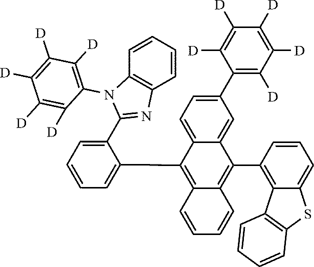 [SMILES: [2H]c1c([2H])c([2H])c(-c2ccc3c(-c4ccccc4-c4nc5ccccc5n4-c4c([2H])c([2H])c([2H])c([2H])c4[2H])c4ccccc4c(-c4cccc5sc6ccccc6c45)c3c2)c([2H])c1[2H]]

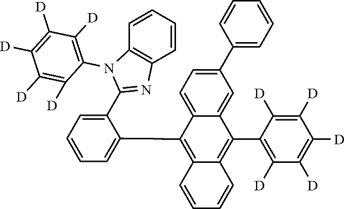 [SMILES: [2H]c1c([2H])c([2H])c(-c2c3ccccc3c(-c3ccccc3-c3nc4ccccc4n3-c3c([2H])c([2H])c([2H])c([2H])c3[2H])c3ccc(-c4ccccc4)cc23)c([2H])c1[2H]]

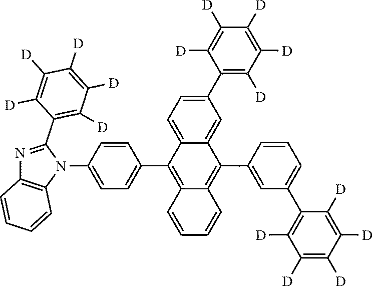 [SMILES: [2H]c1c([2H])c([2H])c(-c2cccc(-c3c4ccccc4c(-c4ccc(-n5c(-c6c([2H])c([2H])c([2H])c([2H])c6[2H])nc6ccccc65)cc4)c4ccc(-c5c([2H])c([2H])c([2H])c([2H])c5[2H])cc34)c2)c([2H])c1[2H]]

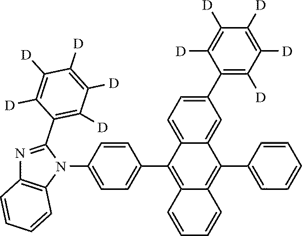 [SMILES: [2H]c1c([2H])c([2H])c(-c2ccc3c(-c4ccc(-n5c(-c6c([2H])c([2H])c([2H])c([2H])c6[2H])nc6ccccc65)cc4)c4ccccc4c(-c4ccccc4)c3c2)c([2H])c1[2H]]

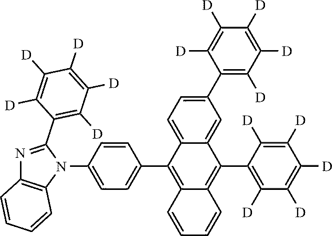 [SMILES: [2H]c1c([2H])c([2H])c(-c2ccc3c(-c4ccc(-n5c(-c6c([2H])c([2H])c([2H])c([2H])c6[2H])nc6ccccc65)cc4)c4ccccc4c(-c4c([2H])c([2H])c([2H])c([2H])c4[2H])c3c2)c([2H])c1[2H]]

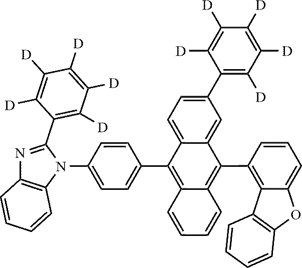 [SMILES: [2H]c1c([2H])c([2H])c(-c2ccc3c(-c4ccc(-n5c(-c6c([2H])c([2H])c([2H])c([2H])c6[2H])nc6ccccc65)cc4)c4ccccc4c(-c4cccc5oc6ccccc6c45)c3c2)c([2H])c1[2H]]

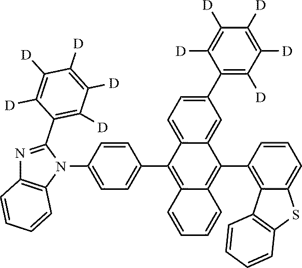 [SMILES: [2H]c1c([2H])c([2H])c(-c2ccc3c(-c4ccc(-n5c(-c6c([2H])c([2H])c([2H])c([2H])c6[2H])nc6ccccc65)cc4)c4ccccc4c(-c4cccc5sc6ccccc6c45)c3c2)c([2H])c1[2H]]

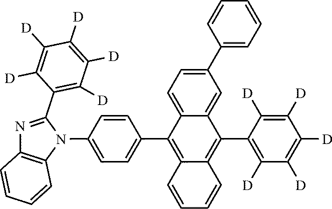 [SMILES: [2H]c1c([2H])c([2H])c(-c2c3ccccc3c(-c3ccc(-n4c(-c5c([2H])c([2H])c([2H])c([2H])c5[2H])nc5ccccc54)cc3)c3ccc(-c4ccccc4)cc23)c([2H])c1[2H]]

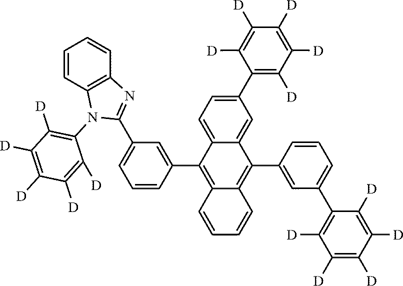 [SMILES: [2H]c1c([2H])c([2H])c(-c2cccc(-c3c4ccccc4c(-c4cccc(-c5nc6ccccc6n5-c5c([2H])c([2H])c([2H])c([2H])c5[2H])c4)c4ccc(-c5c([2H])c([2H])c([2H])c([2H])c5[2H])cc34)c2)c([2H])c1[2H]]

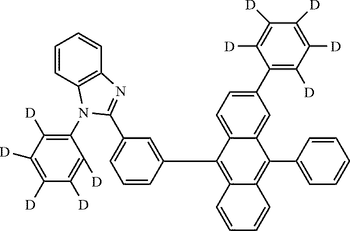 [SMILES: [2H]c1c([2H])c([2H])c(-c2ccc3c(-c4cccc(-c5nc6ccccc6n5-c5c([2H])c([2H])c([2H])c([2H])c5[2H])c4)c4ccccc4c(-c4ccccc4)c3c2)c([2H])c1[2H]]

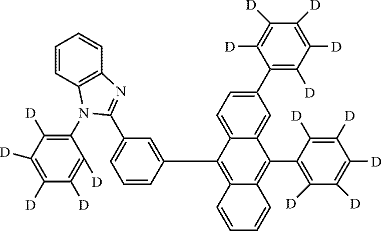 [SMILES: [2H]c1c([2H])c([2H])c(-c2ccc3c(-c4cccc(-c5nc6ccccc6n5-c5c([2H])c([2H])c([2H])c([2H])c5[2H])c4)c4ccccc4c(-c4c([2H])c([2H])c([2H])c([2H])c4[2H])c3c2)c([2H])c1[2H]]

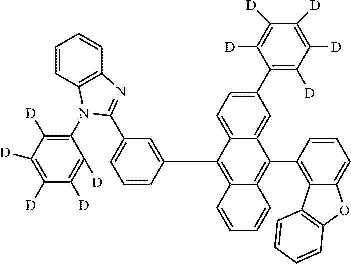 [SMILES: [2H]c1c([2H])c([2H])c(-c2ccc3c(-c4cccc(-c5nc6ccccc6n5-c5c([2H])c([2H])c([2H])c([2H])c5[2H])c4)c4ccccc4c(-c4cccc5oc6ccccc6c45)c3c2)c([2H])c1[2H]]

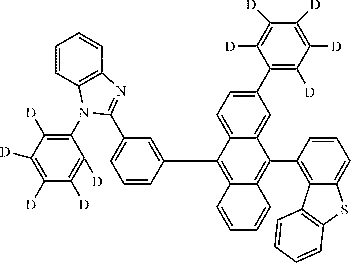 [SMILES: [2H]c1c([2H])c([2H])c(-c2ccc3c(-c4cccc(-c5nc6ccccc6n5-c5c([2H])c([2H])c([2H])c([2H])c5[2H])c4)c4ccccc4c(-c4cccc5sc6ccccc6c45)c3c2)c([2H])c1[2H]]

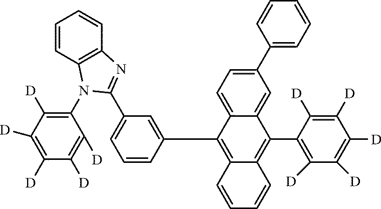 [SMILES: [2H]c1c([2H])c([2H])c(-c2c3ccccc3c(-c3cccc(-c4nc5ccccc5n4-c4c([2H])c([2H])c([2H])c([2H])c4[2H])c3)c3ccc(-c4ccccc4)cc23)c([2H])c1[2H]]

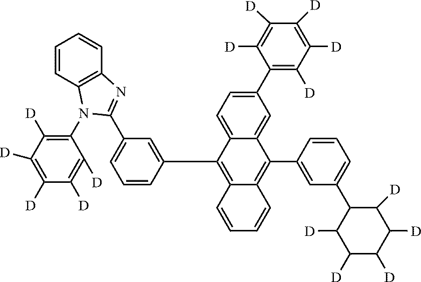 [SMILES: [2H]c1c([2H])c([2H])c(-c2ccc3c(-c4cccc(-c5nc6ccccc6n5-c5c([2H])c([2H])c([2H])c([2H])c5[2H])c4)c4ccccc4c(-c4cccc(C5C([2H])C([2H])C([2H])C([2H])C5[2H])c4)c3c2)c([2H])c1[2H]]

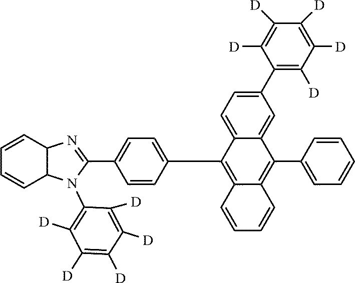 [SMILES: [2H]c1c([2H])c([2H])c(-c2ccc3c(-c4ccc(C5=NC6C=CC=CC6N5c5c([2H])c([2H])c([2H])c([2H])c5[2H])cc4)c4ccccc4c(-c4ccccc4)c3c2)c([2H])c1[2H]]

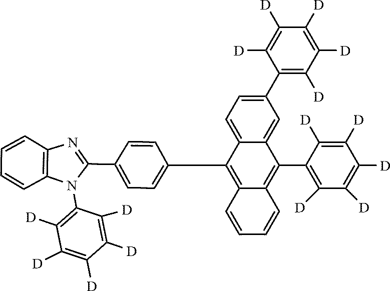 [SMILES: [2H]c1c([2H])c([2H])c(-c2ccc3c(-c4ccc(-c5nc6ccccc6n5-c5c([2H])c([2H])c([2H])c([2H])c5[2H])cc4)c4ccccc4c(-c4c([2H])c([2H])c([2H])c([2H])c4[2H])c3c2)c([2H])c1[2H]]

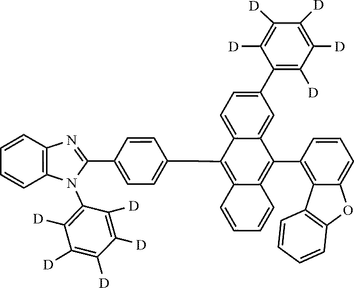 [SMILES: [2H]c1c([2H])c([2H])c(-c2ccc3c(-c4ccc(-c5nc6ccccc6n5-c5c([2H])c([2H])c([2H])c([2H])c5[2H])cc4)c4ccccc4c(-c4cccc5oc6ccccc6c45)c3c2)c([2H])c1[2H]]

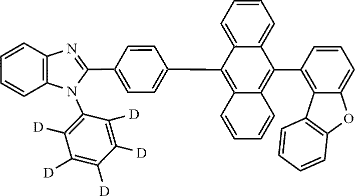 [SMILES: [2H]c1c([2H])c([2H])c(-n2c(-c3ccc(-c4c5ccccc5c(-c5cccc6oc7ccccc7c56)c5ccccc45)cc3)nc3ccccc32)c([2H])c1[2H]]

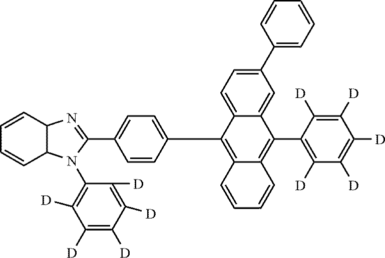 [SMILES: [2H]c1c([2H])c([2H])c(-c2c3ccccc3c(-c3ccc(C4=NC5C=CC=CC5N4c4c([2H])c([2H])c([2H])c([2H])c4[2H])cc3)c3ccc(-c4ccccc4)cc23)c([2H])c1[2H]]

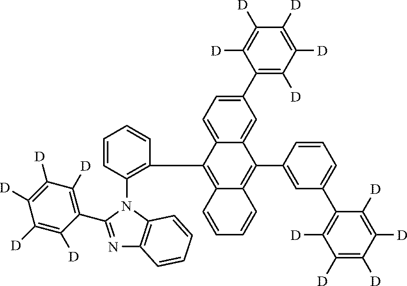 [SMILES: [2H]c1c([2H])c([2H])c(-c2cccc(-c3c4ccccc4c(-c4ccccc4-n4c(-c5c([2H])c([2H])c([2H])c([2H])c5[2H])nc5ccccc54)c4ccc(-c5c([2H])c([2H])c([2H])c([2H])c5[2H])cc34)c2)c([2H])c1[2H]]

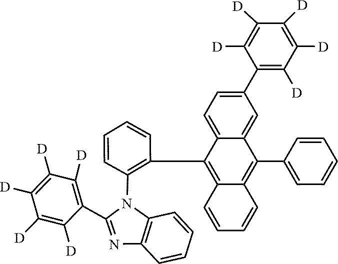 [SMILES: [2H]c1c([2H])c([2H])c(-c2ccc3c(-c4ccccc4-n4c(-c5c([2H])c([2H])c([2H])c([2H])c5[2H])nc5ccccc54)c4ccccc4c(-c4ccccc4)c3c2)c([2H])c1[2H]]

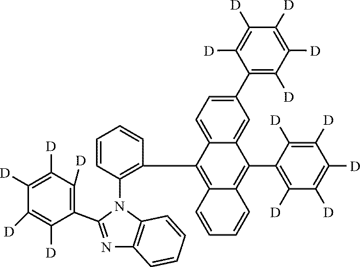 [SMILES: [2H]c1c([2H])c([2H])c(-c2ccc3c(-c4ccccc4-n4c(-c5c([2H])c([2H])c([2H])c([2H])c5[2H])nc5ccccc54)c4ccccc4c(-c4c([2H])c([2H])c([2H])c([2H])c4[2H])c3c2)c([2H])c1[2H]]

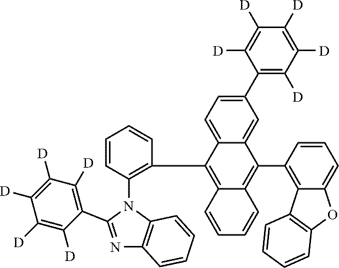 [SMILES: [2H]c1c([2H])c([2H])c(-c2ccc3c(-c4ccccc4-n4c(-c5c([2H])c([2H])c([2H])c([2H])c5[2H])nc5ccccc54)c4ccccc4c(-c4cccc5oc6ccccc6c45)c3c2)c([2H])c1[2H]]

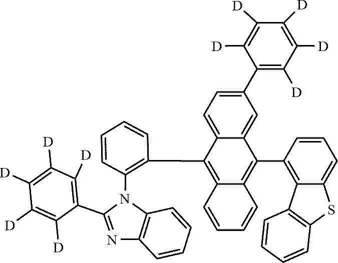 [SMILES: [2H]c1c([2H])c([2H])c(-c2ccc3c(-c4ccccc4-n4c(-c5c([2H])c([2H])c([2H])c([2H])c5[2H])nc5ccccc54)c4ccccc4c(-c4cccc5sc6ccccc6c45)c3c2)c([2H])c1[2H]]

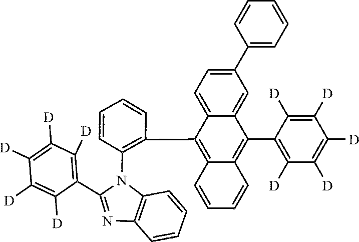 [SMILES: [2H]c1c([2H])c([2H])c(-c2c3ccccc3c(-c3ccccc3-n3c(-c4c([2H])c([2H])c([2H])c([2H])c4[2H])nc4ccccc43)c3ccc(-c4ccccc4)cc23)c([2H])c1[2H]]

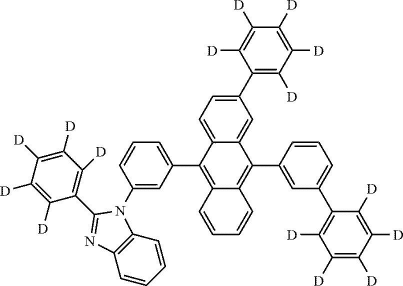 [SMILES: [2H]c1c([2H])c([2H])c(-c2cccc(-c3c4ccccc4c(-c4cccc(-n5c(-c6c([2H])c([2H])c([2H])c([2H])c6[2H])nc6ccccc65)c4)c4ccc(-c5c([2H])c([2H])c([2H])c([2H])c5[2H])cc34)c2)c([2H])c1[2H]]

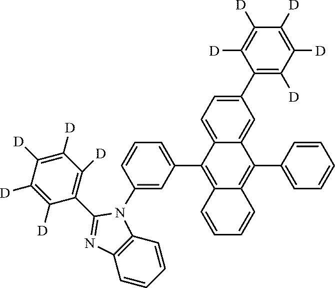 [SMILES: [2H]c1c([2H])c([2H])c(-c2ccc3c(-c4cccc(-n5c(-c6c([2H])c([2H])c([2H])c([2H])c6[2H])nc6ccccc65)c4)c4ccccc4c(-c4ccccc4)c3c2)c([2H])c1[2H]]

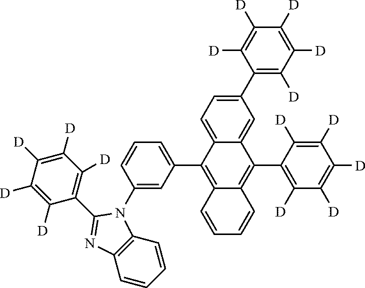 [SMILES: [2H]c1c([2H])c([2H])c(-c2ccc3c(-c4cccc(-n5c(-c6c([2H])c([2H])c([2H])c([2H])c6[2H])nc6ccccc65)c4)c4ccccc4c(-c4c([2H])c([2H])c([2H])c([2H])c4[2H])c3c2)c([2H])c1[2H]]

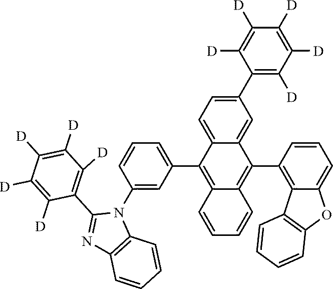 [SMILES: [2H]c1c([2H])c([2H])c(-c2ccc3c(-c4cccc(-n5c(-c6c([2H])c([2H])c([2H])c([2H])c6[2H])nc6ccccc65)c4)c4ccccc4c(-c4cccc5oc6ccccc6c45)c3c2)c([2H])c1[2H]]

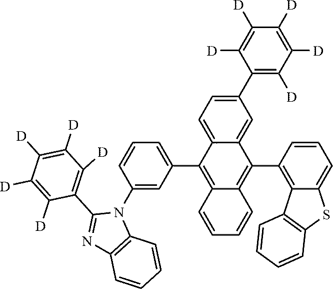 [SMILES: [2H]c1c([2H])c([2H])c(-c2ccc3c(-c4cccc(-n5c(-c6c([2H])c([2H])c([2H])c([2H])c6[2H])nc6ccccc65)c4)c4ccccc4c(-c4cccc5sc6ccccc6c45)c3c2)c([2H])c1[2H]]

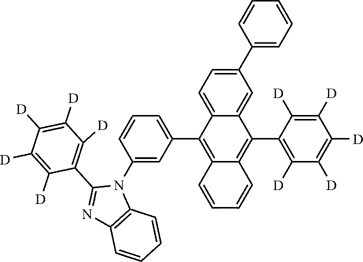 [SMILES: [2H]c1c([2H])c([2H])c(-c2c3ccccc3c(-c3cccc(-n4c(-c5c([2H])c([2H])c([2H])c([2H])c5[2H])nc5ccccc54)c3)c3ccc(-c4ccccc4)cc23)c([2H])c1[2H]]

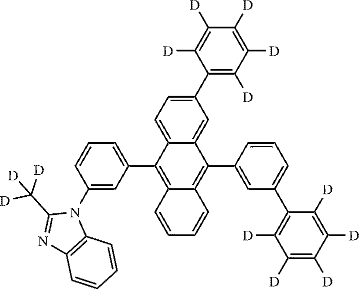 [SMILES: [2H]c1c([2H])c([2H])c(-c2cccc(-c3c4ccccc4c(-c4cccc(-n5c(C([2H])([2H])[2H])nc6ccccc65)c4)c4ccc(-c5c([2H])c([2H])c([2H])c([2H])c5[2H])cc34)c2)c([2H])c1[2H]]

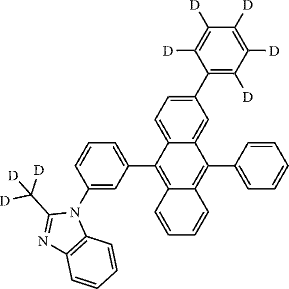 [SMILES: [2H]c1c([2H])c([2H])c(-c2ccc3c(-c4cccc(-n5c(C([2H])([2H])[2H])nc6ccccc65)c4)c4ccccc4c(-c4ccccc4)c3c2)c([2H])c1[2H]]

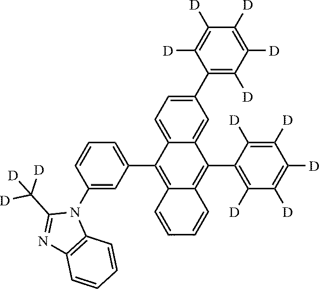 [SMILES: [2H]c1c([2H])c([2H])c(-c2ccc3c(-c4cccc(-n5c(C([2H])([2H])[2H])nc6ccccc65)c4)c4ccccc4c(-c4c([2H])c([2H])c([2H])c([2H])c4[2H])c3c2)c([2H])c1[2H]]

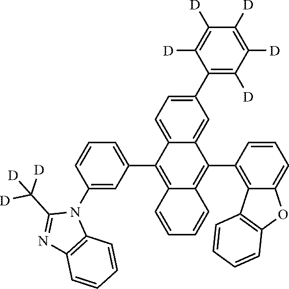 [SMILES: [2H]c1c([2H])c([2H])c(-c2ccc3c(-c4cccc(-n5c(C([2H])([2H])[2H])nc6ccccc65)c4)c4ccccc4c(-c4cccc5oc6ccccc6c45)c3c2)c([2H])c1[2H]]

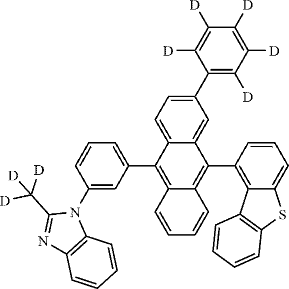 [SMILES: [2H]c1c([2H])c([2H])c(-c2ccc3c(-c4cccc(-n5c(C([2H])([2H])[2H])nc6ccccc65)c4)c4ccccc4c(-c4cccc5sc6ccccc6c45)c3c2)c([2H])c1[2H]]

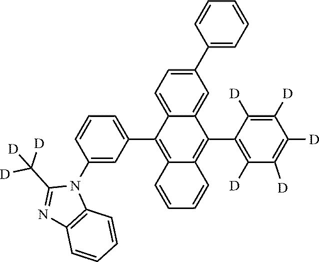 [SMILES: [2H]c1c([2H])c([2H])c(-c2c3ccccc3c(-c3cccc(-n4c(C([2H])([2H])[2H])nc5ccccc54)c3)c3ccc(-c4ccccc4)cc23)c([2H])c1[2H]]

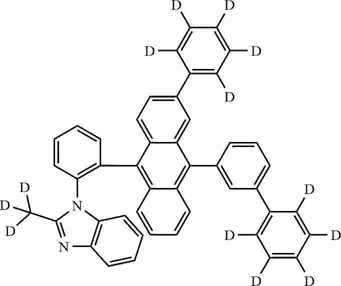 [SMILES: [2H]c1c([2H])c([2H])c(-c2cccc(-c3c4ccccc4c(-c4ccccc4-n4c(C([2H])([2H])[2H])nc5ccccc54)c4ccc(-c5c([2H])c([2H])c([2H])c([2H])c5[2H])cc34)c2)c([2H])c1[2H]]

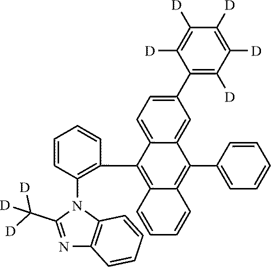 [SMILES: [2H]c1c([2H])c([2H])c(-c2ccc3c(-c4ccccc4-n4c(C([2H])([2H])[2H])nc5ccccc54)c4ccccc4c(-c4ccccc4)c3c2)c([2H])c1[2H]]

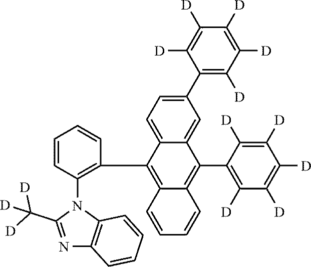 [SMILES: [2H]c1c([2H])c([2H])c(-c2ccc3c(-c4ccccc4-n4c(C([2H])([2H])[2H])nc5ccccc54)c4ccccc4c(-c4c([2H])c([2H])c([2H])c([2H])c4[2H])c3c2)c([2H])c1[2H]]

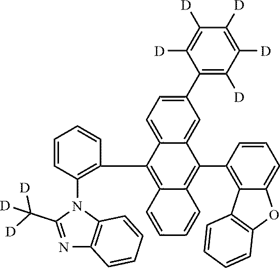 [SMILES: [2H]c1c([2H])c([2H])c(-c2ccc3c(-c4ccccc4-n4c(C([2H])([2H])[2H])nc5ccccc54)c4ccccc4c(-c4cccc5oc6ccccc6c45)c3c2)c([2H])c1[2H]]

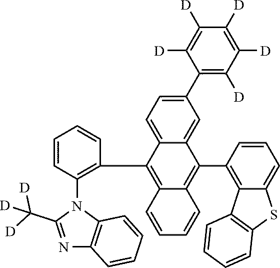 [SMILES: [2H]c1c([2H])c([2H])c(-c2ccc3c(-c4ccccc4-n4c(C([2H])([2H])[2H])nc5ccccc54)c4ccccc4c(-c4cccc5sc6ccccc6c45)c3c2)c([2H])c1[2H]]

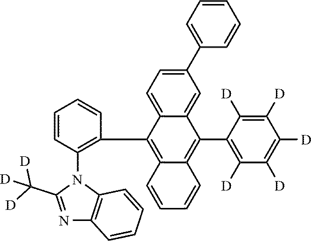 [SMILES: [2H]c1c([2H])c([2H])c(-c2c3ccccc3c(-c3ccccc3-n3c(C([2H])([2H])[2H])nc4ccccc43)c3ccc(-c4ccccc4)cc23)c([2H])c1[2H]]

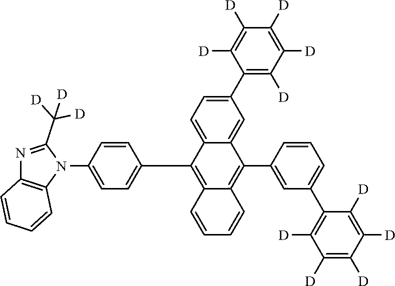 [SMILES: [2H]c1c([2H])c([2H])c(-c2cccc(-c3c4ccccc4c(-c4ccc(-n5c(C([2H])([2H])[2H])nc6ccccc65)cc4)c4ccc(-c5c([2H])c([2H])c([2H])c([2H])c5[2H])cc34)c2)c([2H])c1[2H]]

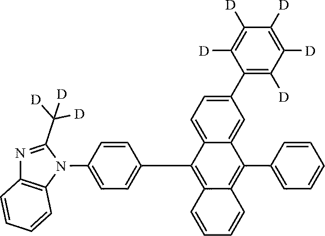 [SMILES: [2H]c1c([2H])c([2H])c(-c2ccc3c(-c4ccc(-n5c(C([2H])([2H])[2H])nc6ccccc65)cc4)c4ccccc4c(-c4ccccc4)c3c2)c([2H])c1[2H]]

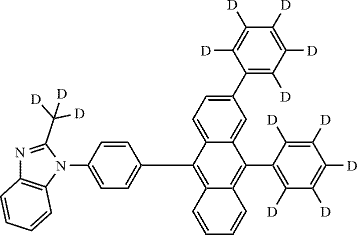 [SMILES: [2H]c1c([2H])c([2H])c(-c2ccc3c(-c4ccc(-n5c(C([2H])([2H])[2H])nc6ccccc65)cc4)c4ccccc4c(-c4c([2H])c([2H])c([2H])c([2H])c4[2H])c3c2)c([2H])c1[2H]]

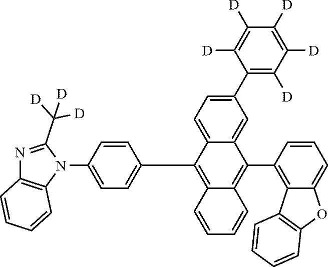 [SMILES: [2H]c1c([2H])c([2H])c(-c2ccc3c(-c4ccc(-n5c(C([2H])([2H])[2H])nc6ccccc65)cc4)c4ccccc4c(-c4cccc5oc6ccccc6c45)c3c2)c([2H])c1[2H]]

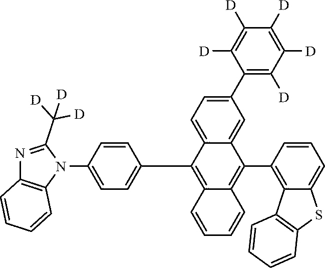 [SMILES: [2H]c1c([2H])c([2H])c(-c2ccc3c(-c4ccc(-n5c(C([2H])([2H])[2H])nc6ccccc65)cc4)c4ccccc4c(-c4cccc5sc6ccccc6c45)c3c2)c([2H])c1[2H]]

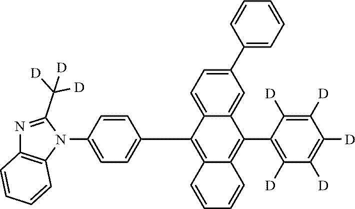 [SMILES: [2H]c1c([2H])c([2H])c(-c2c3ccccc3c(-c3ccc(-n4c(C([2H])([2H])[2H])nc5ccccc54)cc3)c3ccc(-c4ccccc4)cc23)c([2H])c1[2H]]

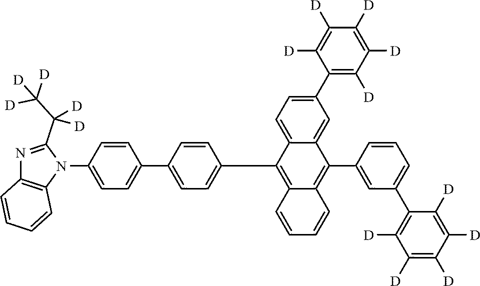 [SMILES: [2H]c1c([2H])c([2H])c(-c2cccc(-c3c4ccccc4c(-c4ccc(-c5ccc(-n6c(C([2H])([2H])C([2H])([2H])[2H])nc7ccccc76)cc5)cc4)c4ccc(-c5c([2H])c([2H])c([2H])c([2H])c5[2H])cc34)c2)c([2H])c1[2H]]

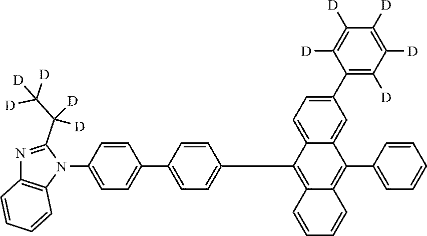 [SMILES: [2H]c1c([2H])c([2H])c(-c2ccc3c(-c4ccc(-c5ccc(-n6c(C([2H])([2H])C([2H])([2H])[2H])nc7ccccc76)cc5)cc4)c4ccccc4c(-c4ccccc4)c3c2)c([2H])c1[2H]]

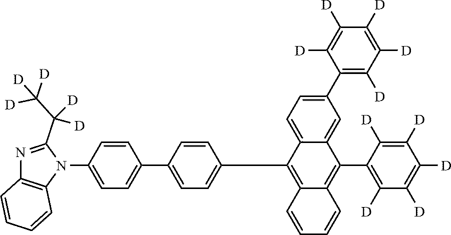 [SMILES: [2H]c1c([2H])c([2H])c(-c2ccc3c(-c4ccc(-c5ccc(-n6c(C([2H])([2H])C([2H])([2H])[2H])nc7ccccc76)cc5)cc4)c4ccccc4c(-c4c([2H])c([2H])c([2H])c([2H])c4[2H])c3c2)c([2H])c1[2H]]

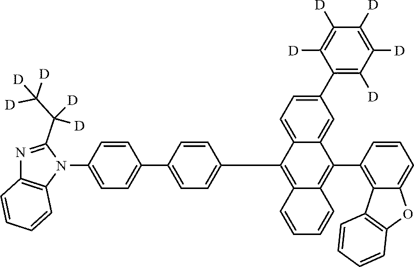 [SMILES: [2H]c1c([2H])c([2H])c(-c2ccc3c(-c4ccc(-c5ccc(-n6c(C([2H])([2H])C([2H])([2H])[2H])nc7ccccc76)cc5)cc4)c4ccccc4c(-c4cccc5oc6ccccc6c45)c3c2)c([2H])c1[2H]]

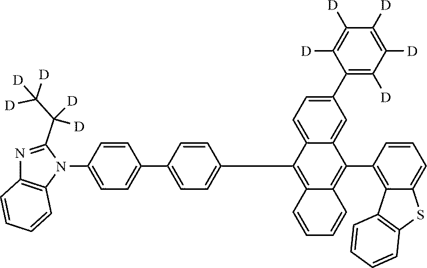 [SMILES: [2H]c1c([2H])c([2H])c(-c2ccc3c(-c4ccc(-c5ccc(-n6c(C([2H])([2H])C([2H])([2H])[2H])nc7ccccc76)cc5)cc4)c4ccccc4c(-c4cccc5sc6ccccc6c45)c3c2)c([2H])c1[2H]]

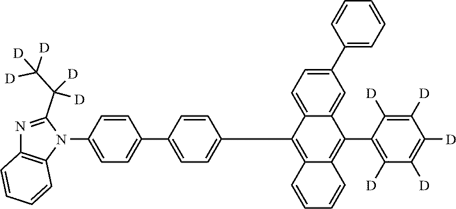 [SMILES: [2H]c1c([2H])c([2H])c(-c2c3ccccc3c(-c3ccc(-c4ccc(-n5c(C([2H])([2H])C([2H])([2H])[2H])nc6ccccc65)cc4)cc3)c3ccc(-c4ccccc4)cc23)c([2H])c1[2H]]

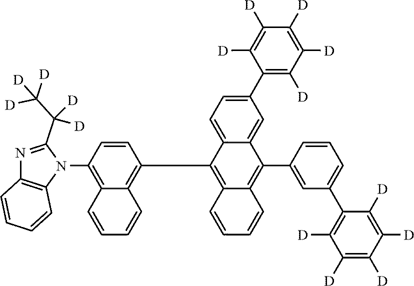 [SMILES: [2H]c1c([2H])c([2H])c(-c2cccc(-c3c4ccccc4c(-c4ccc(-n5c(C([2H])([2H])C([2H])([2H])[2H])nc6ccccc65)c5ccccc45)c4ccc(-c5c([2H])c([2H])c([2H])c([2H])c5[2H])cc34)c2)c([2H])c1[2H]]

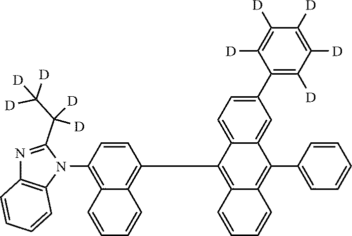 [SMILES: [2H]c1c([2H])c([2H])c(-c2ccc3c(-c4ccc(-n5c(C([2H])([2H])C([2H])([2H])[2H])nc6ccccc65)c5ccccc45)c4ccccc4c(-c4ccccc4)c3c2)c([2H])c1[2H]]

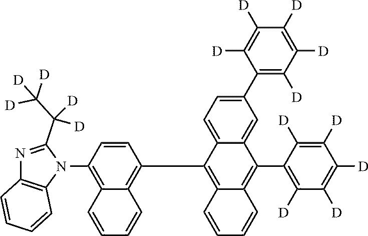 [SMILES: [2H]c1c([2H])c([2H])c(-c2ccc3c(-c4ccc(-n5c(C([2H])([2H])C([2H])([2H])[2H])nc6ccccc65)c5ccccc45)c4ccccc4c(-c4c([2H])c([2H])c([2H])c([2H])c4[2H])c3c2)c([2H])c1[2H]]

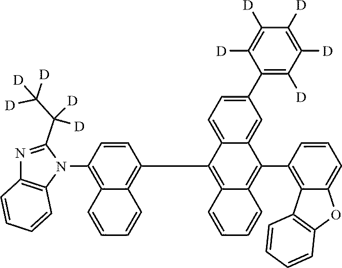 [SMILES: [2H]c1c([2H])c([2H])c(-c2ccc3c(-c4ccc(-n5c(C([2H])([2H])C([2H])([2H])[2H])nc6ccccc65)c5ccccc45)c4ccccc4c(-c4cccc5oc6ccccc6c45)c3c2)c([2H])c1[2H]]

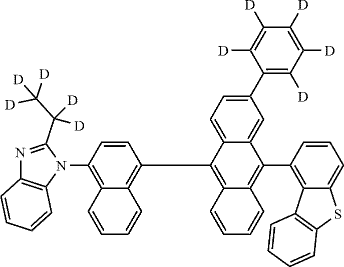 [SMILES: [2H]c1c([2H])c([2H])c(-c2ccc3c(-c4ccc(-n5c(C([2H])([2H])C([2H])([2H])[2H])nc6ccccc65)c5ccccc45)c4ccccc4c(-c4cccc5sc6ccccc6c45)c3c2)c([2H])c1[2H]]

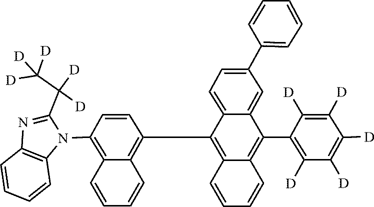 [SMILES: [2H]c1c([2H])c([2H])c(-c2c3ccccc3c(-c3ccc(-n4c(C([2H])([2H])C([2H])([2H])[2H])nc5ccccc54)c4ccccc34)c3ccc(-c4ccccc4)cc23)c([2H])c1[2H]]

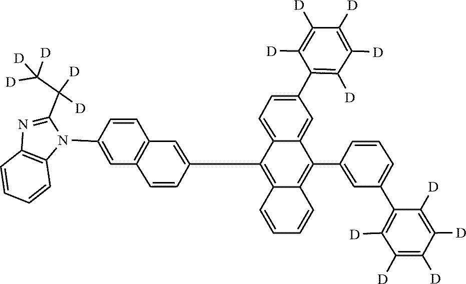 [SMILES: [2H]c1c([2H])c([2H])c(-c2cccc(-c3c4ccccc4c(-c4ccc5cc(-n6c(C([2H])([2H])C([2H])([2H])[2H])nc7ccccc76)ccc5c4)c4ccc(-c5c([2H])c([2H])c([2H])c([2H])c5[2H])cc34)c2)c([2H])c1[2H]]

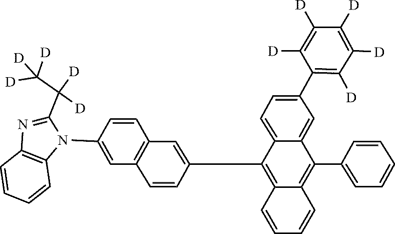 [SMILES: [2H]c1c([2H])c([2H])c(-c2ccc3c(-c4ccc5cc(-n6c(C([2H])([2H])C([2H])([2H])[2H])nc7ccccc76)ccc5c4)c4ccccc4c(-c4ccccc4)c3c2)c([2H])c1[2H]]

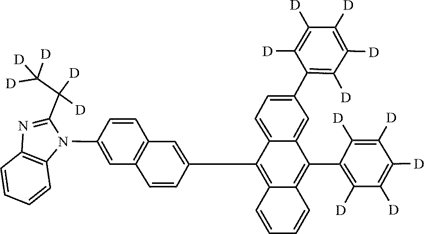 [SMILES: [2H]c1c([2H])c([2H])c(-c2ccc3c(-c4ccc5cc(-n6c(C([2H])([2H])C([2H])([2H])[2H])nc7ccccc76)ccc5c4)c4ccccc4c(-c4c([2H])c([2H])c([2H])c([2H])c4[2H])c3c2)c([2H])c1[2H]]

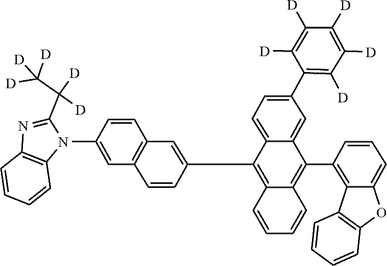 [SMILES: [2H]c1c([2H])c([2H])c(-c2ccc3c(-c4ccc5cc(-n6c(C([2H])([2H])C([2H])([2H])[2H])nc7ccccc76)ccc5c4)c4ccccc4c(-c4cccc5oc6ccccc6c45)c3c2)c([2H])c1[2H]]

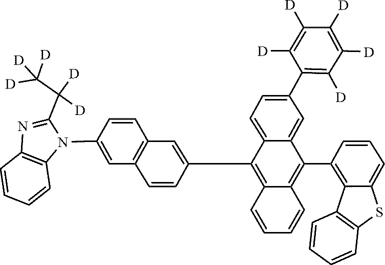 [SMILES: [2H]c1c([2H])c([2H])c(-c2ccc3c(-c4ccc5cc(-n6c(C([2H])([2H])C([2H])([2H])[2H])nc7ccccc76)ccc5c4)c4ccccc4c(-c4cccc5sc6ccccc6c45)c3c2)c([2H])c1[2H]]

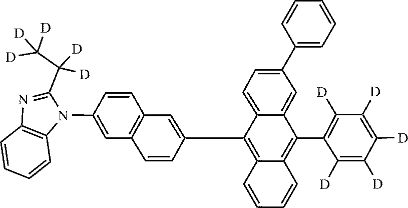 [SMILES: [2H]c1c([2H])c([2H])c(-c2c3ccccc3c(-c3ccc4cc(-n5c(C([2H])([2H])C([2H])([2H])[2H])nc6ccccc65)ccc4c3)c3ccc(-c4ccccc4)cc23)c([2H])c1[2H]]

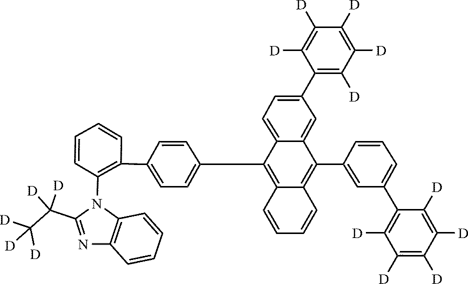 [SMILES: [2H]c1c([2H])c([2H])c(-c2cccc(-c3c4ccccc4c(-c4ccc(-c5ccccc5-n5c(C([2H])([2H])C([2H])([2H])[2H])nc6ccccc65)cc4)c4ccc(-c5c([2H])c([2H])c([2H])c([2H])c5[2H])cc34)c2)c([2H])c1[2H]]

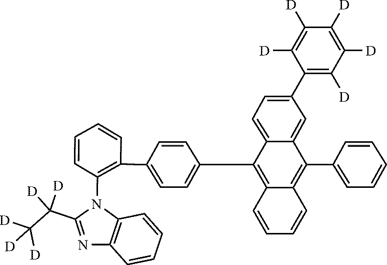 [SMILES: [2H]c1c([2H])c([2H])c(-c2ccc3c(-c4ccc(-c5ccccc5-n5c(C([2H])([2H])C([2H])([2H])[2H])nc6ccccc65)cc4)c4ccccc4c(-c4ccccc4)c3c2)c([2H])c1[2H]]

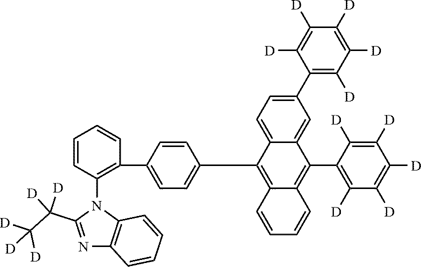 [SMILES: [2H]c1c([2H])c([2H])c(-c2ccc3c(-c4ccc(-c5ccccc5-n5c(C([2H])([2H])C([2H])([2H])[2H])nc6ccccc65)cc4)c4ccccc4c(-c4c([2H])c([2H])c([2H])c([2H])c4[2H])c3c2)c([2H])c1[2H]]

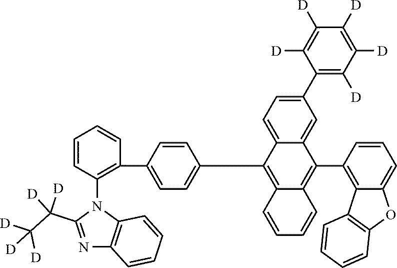 [SMILES: [2H]c1c([2H])c([2H])c(-c2ccc3c(-c4ccc(-c5ccccc5-n5c(C([2H])([2H])C([2H])([2H])[2H])nc6ccccc65)cc4)c4ccccc4c(-c4cccc5oc6ccccc6c45)c3c2)c([2H])c1[2H]]

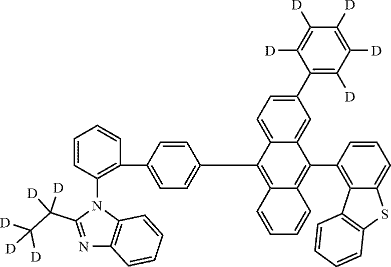 [SMILES: [2H]c1c([2H])c([2H])c(-c2ccc3c(-c4ccc(-c5ccccc5-n5c(C([2H])([2H])C([2H])([2H])[2H])nc6ccccc65)cc4)c4ccccc4c(-c4cccc5sc6ccccc6c45)c3c2)c([2H])c1[2H]]

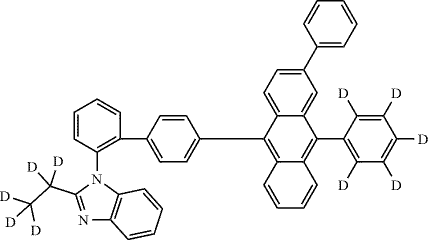 [SMILES: [2H]c1c([2H])c([2H])c(-c2c3ccccc3c(-c3ccc(-c4ccccc4-n4c(C([2H])([2H])C([2H])([2H])[2H])nc5ccccc54)cc3)c3ccc(-c4ccccc4)cc23)c([2H])c1[2H]]

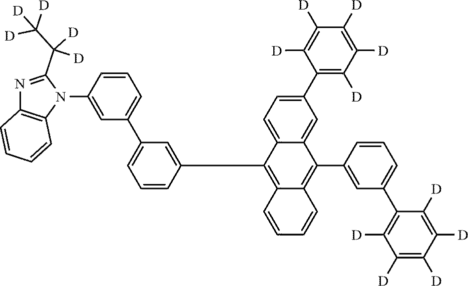 [SMILES: [2H]c1c([2H])c([2H])c(-c2cccc(-c3c4ccccc4c(-c4cccc(-c5cccc(-n6c(C([2H])([2H])C([2H])([2H])[2H])nc7ccccc76)c5)c4)c4ccc(-c5c([2H])c([2H])c([2H])c([2H])c5[2H])cc34)c2)c([2H])c1[2H]]

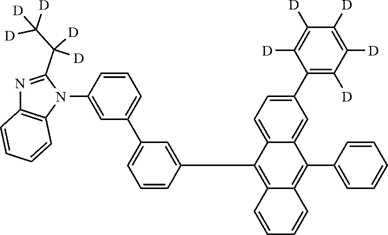 [SMILES: [2H]c1c([2H])c([2H])c(-c2ccc3c(-c4cccc(-c5cccc(-n6c(C([2H])([2H])C([2H])([2H])[2H])nc7ccccc76)c5)c4)c4ccccc4c(-c4ccccc4)c3c2)c([2H])c1[2H]]

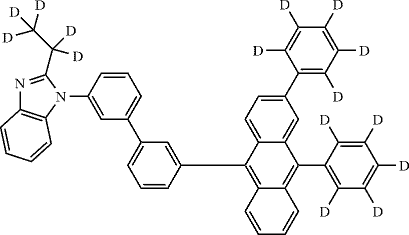 [SMILES: [2H]c1c([2H])c([2H])c(-c2ccc3c(-c4cccc(-c5cccc(-n6c(C([2H])([2H])C([2H])([2H])[2H])nc7ccccc76)c5)c4)c4ccccc4c(-c4c([2H])c([2H])c([2H])c([2H])c4[2H])c3c2)c([2H])c1[2H]]

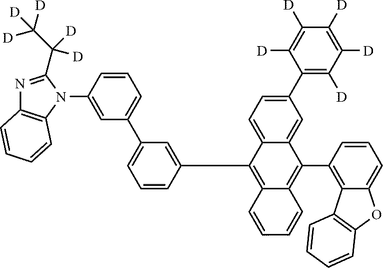 [SMILES: [2H]c1c([2H])c([2H])c(-c2ccc3c(-c4cccc(-c5cccc(-n6c(C([2H])([2H])C([2H])([2H])[2H])nc7ccccc76)c5)c4)c4ccccc4c(-c4cccc5oc6ccccc6c45)c3c2)c([2H])c1[2H]]

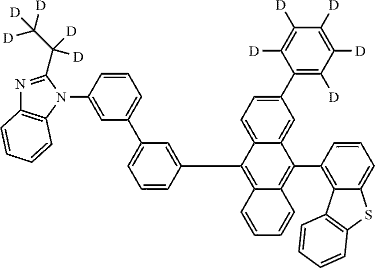 [SMILES: [2H]c1c([2H])c([2H])c(-c2ccc3c(-c4cccc(-c5cccc(-n6c(C([2H])([2H])C([2H])([2H])[2H])nc7ccccc76)c5)c4)c4ccccc4c(-c4cccc5sc6ccccc6c45)c3c2)c([2H])c1[2H]]

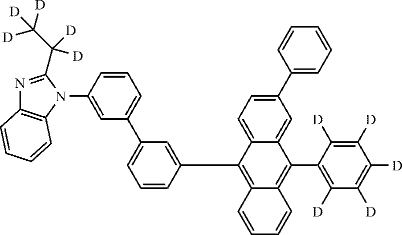 [SMILES: [2H]c1c([2H])c([2H])c(-c2c3ccccc3c(-c3cccc(-c4cccc(-n5c(C([2H])([2H])C([2H])([2H])[2H])nc6ccccc65)c4)c3)c3ccc(-c4ccccc4)cc23)c([2H])c1[2H]]

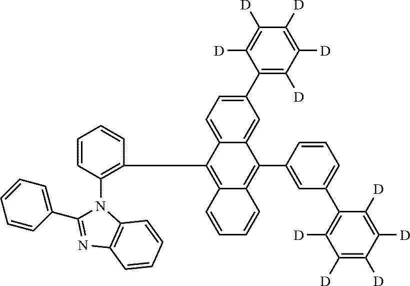 [SMILES: [2H]c1c([2H])c([2H])c(-c2cccc(-c3c4ccccc4c(-c4ccccc4-n4c(-c5ccccc5)nc5ccccc54)c4ccc(-c5c([2H])c([2H])c([2H])c([2H])c5[2H])cc34)c2)c([2H])c1[2H]]

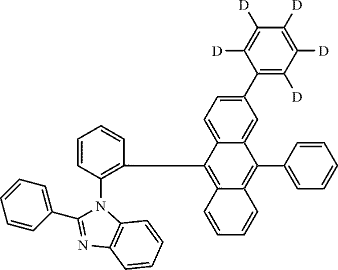 [SMILES: [2H]c1c([2H])c([2H])c(-c2ccc3c(-c4ccccc4-n4c(-c5ccccc5)nc5ccccc54)c4ccccc4c(-c4ccccc4)c3c2)c([2H])c1[2H]]

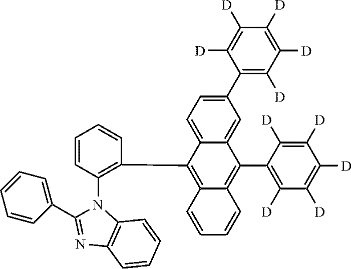 [SMILES: [2H]c1c([2H])c([2H])c(-c2ccc3c(-c4ccccc4-n4c(-c5ccccc5)nc5ccccc54)c4ccccc4c(-c4c([2H])c([2H])c([2H])c([2H])c4[2H])c3c2)c([2H])c1[2H]]

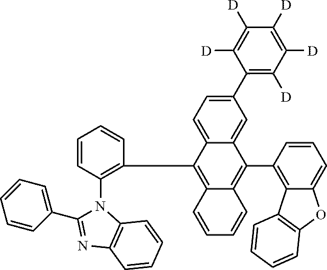 [SMILES: [2H]c1c([2H])c([2H])c(-c2ccc3c(-c4ccccc4-n4c(-c5ccccc5)nc5ccccc54)c4ccccc4c(-c4cccc5oc6ccccc6c45)c3c2)c([2H])c1[2H]]

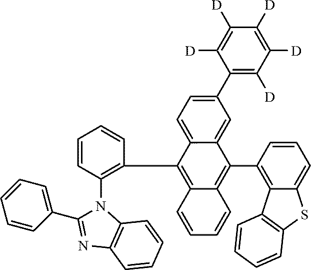 [SMILES: [2H]c1c([2H])c([2H])c(-c2ccc3c(-c4ccccc4-n4c(-c5ccccc5)nc5ccccc54)c4ccccc4c(-c4cccc5sc6ccccc6c45)c3c2)c([2H])c1[2H]]

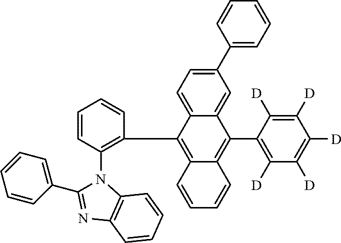 [SMILES: [2H]c1c([2H])c([2H])c(-c2c3ccccc3c(-c3ccccc3-n3c(-c4ccccc4)nc4ccccc43)c3ccc(-c4ccccc4)cc23)c([2H])c1[2H]]

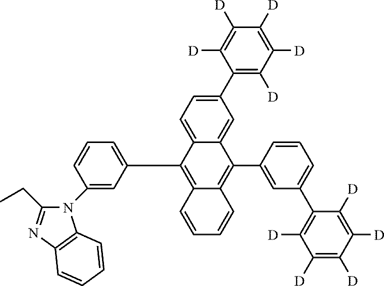 [SMILES: [2H]c1c([2H])c([2H])c(-c2cccc(-c3c4ccccc4c(-c4cccc(-n5c(CC)nc6ccccc65)c4)c4ccc(-c5c([2H])c([2H])c([2H])c([2H])c5[2H])cc34)c2)c([2H])c1[2H]]

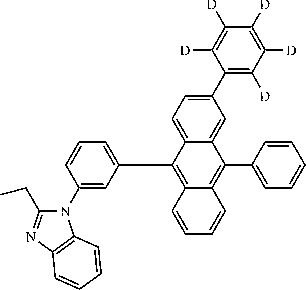 [SMILES: [2H]c1c([2H])c([2H])c(-c2ccc3c(-c4cccc(-n5c(CC)nc6ccccc65)c4)c4ccccc4c(-c4ccccc4)c3c2)c([2H])c1[2H]]

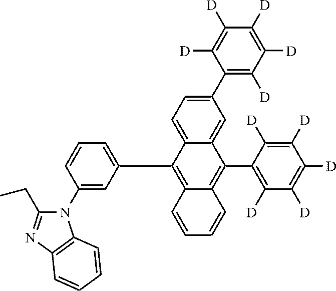 [SMILES: [2H]c1c([2H])c([2H])c(-c2ccc3c(-c4cccc(-n5c(CC)nc6ccccc65)c4)c4ccccc4c(-c4c([2H])c([2H])c([2H])c([2H])c4[2H])c3c2)c([2H])c1[2H]]

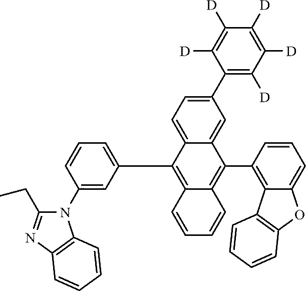 [SMILES: [2H]c1c([2H])c([2H])c(-c2ccc3c(-c4cccc(-n5c(CC)nc6ccccc65)c4)c4ccccc4c(-c4cccc5oc6ccccc6c45)c3c2)c([2H])c1[2H]]

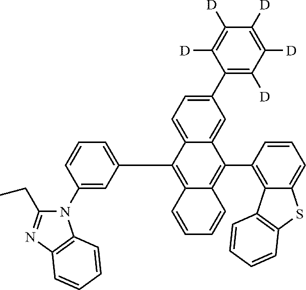 [SMILES: [2H]c1c([2H])c([2H])c(-c2ccc3c(-c4cccc(-n5c(CC)nc6ccccc65)c4)c4ccccc4c(-c4cccc5sc6ccccc6c45)c3c2)c([2H])c1[2H]]

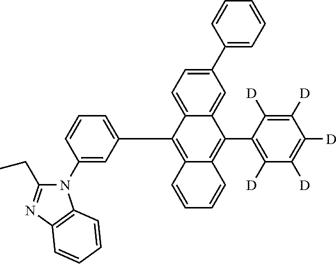 [SMILES: [2H]c1c([2H])c([2H])c(-c2c3ccccc3c(-c3cccc(-n4c(CC)nc5ccccc54)c3)c3ccc(-c4ccccc4)cc23)c([2H])c1[2H]]

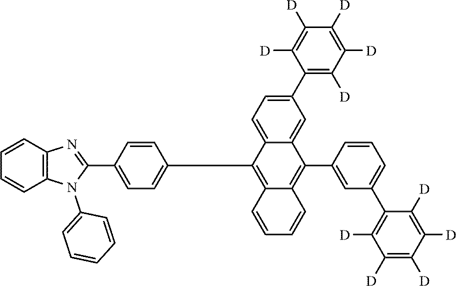 [SMILES: [2H]c1c([2H])c([2H])c(-c2cccc(-c3c4ccccc4c(-c4ccc(-c5nc6ccccc6n5-c5ccccc5)cc4)c4ccc(-c5c([2H])c([2H])c([2H])c([2H])c5[2H])cc34)c2)c([2H])c1[2H]]

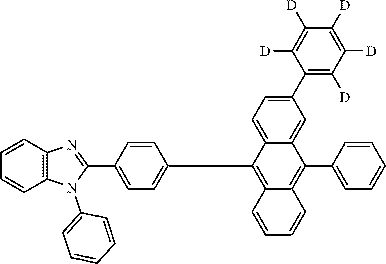 [SMILES: [2H]c1c([2H])c([2H])c(-c2ccc3c(-c4ccc(-c5nc6ccccc6n5-c5ccccc5)cc4)c4ccccc4c(-c4ccccc4)c3c2)c([2H])c1[2H]]

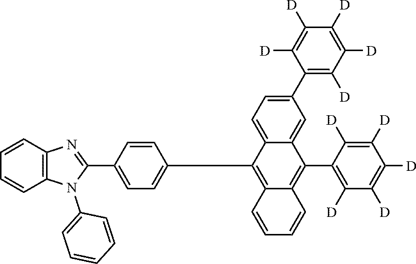 [SMILES: [2H]c1c([2H])c([2H])c(-c2ccc3c(-c4ccc(-c5nc6ccccc6n5-c5ccccc5)cc4)c4ccccc4c(-c4c([2H])c([2H])c([2H])c([2H])c4[2H])c3c2)c([2H])c1[2H]]

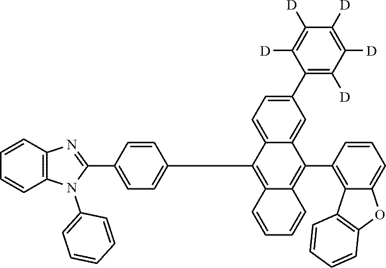 [SMILES: [2H]c1c([2H])c([2H])c(-c2ccc3c(-c4ccc(-c5nc6ccccc6n5-c5ccccc5)cc4)c4ccccc4c(-c4cccc5oc6ccccc6c45)c3c2)c([2H])c1[2H]]

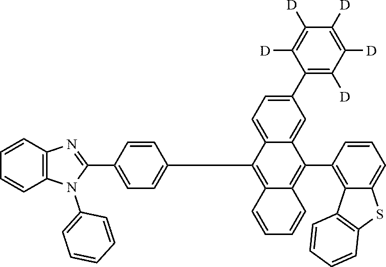 [SMILES: [2H]c1c([2H])c([2H])c(-c2ccc3c(-c4ccc(-c5nc6ccccc6n5-c5ccccc5)cc4)c4ccccc4c(-c4cccc5sc6ccccc6c45)c3c2)c([2H])c1[2H]]

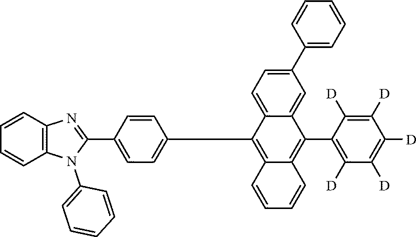 [SMILES: [2H]c1c([2H])c([2H])c(-c2c3ccccc3c(-c3ccc(-c4nc5ccccc5n4-c4ccccc4)cc3)c3ccc(-c4ccccc4)cc23)c([2H])c1[2H]]

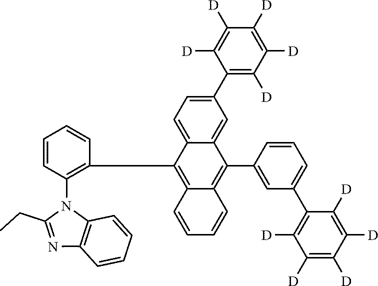 [SMILES: [2H]c1c([2H])c([2H])c(-c2cccc(-c3c4ccccc4c(-c4ccccc4-n4c(CC)nc5ccccc54)c4ccc(-c5c([2H])c([2H])c([2H])c([2H])c5[2H])cc34)c2)c([2H])c1[2H]]

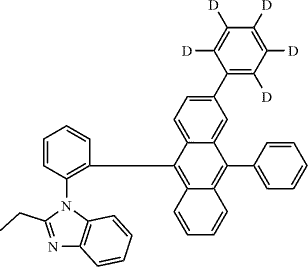 [SMILES: [2H]c1c([2H])c([2H])c(-c2ccc3c(-c4ccccc4-n4c(CC)nc5ccccc54)c4ccccc4c(-c4ccccc4)c3c2)c([2H])c1[2H]]

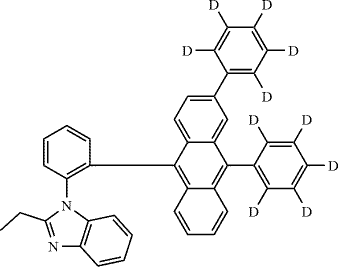 [SMILES: [2H]c1c([2H])c([2H])c(-c2ccc3c(-c4ccccc4-n4c(CC)nc5ccccc54)c4ccccc4c(-c4c([2H])c([2H])c([2H])c([2H])c4[2H])c3c2)c([2H])c1[2H]]

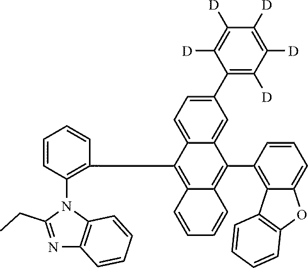 [SMILES: [2H]c1c([2H])c([2H])c(-c2ccc3c(-c4ccccc4-n4c(CC)nc5ccccc54)c4ccccc4c(-c4cccc5oc6ccccc6c45)c3c2)c([2H])c1[2H]]

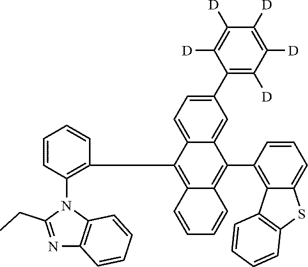 [SMILES: [2H]c1c([2H])c([2H])c(-c2ccc3c(-c4ccccc4-n4c(CC)nc5ccccc54)c4ccccc4c(-c4cccc5sc6ccccc6c45)c3c2)c([2H])c1[2H]]

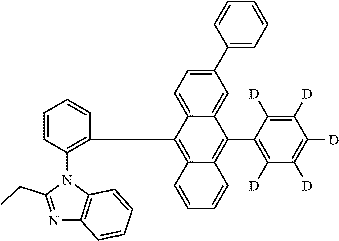 [SMILES: [2H]c1c([2H])c([2H])c(-c2c3ccccc3c(-c3ccccc3-n3c(CC)nc4ccccc43)c3ccc(-c4ccccc4)cc23)c([2H])c1[2H]]

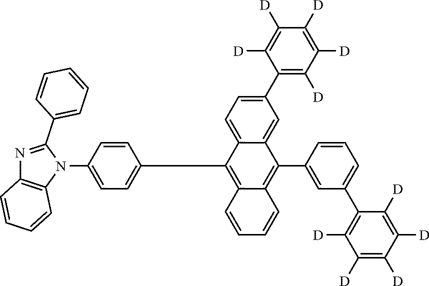 [SMILES: [2H]c1c([2H])c([2H])c(-c2cccc(-c3c4ccccc4c(-c4ccc(-n5c(-c6ccccc6)nc6ccccc65)cc4)c4ccc(-c5c([2H])c([2H])c([2H])c([2H])c5[2H])cc34)c2)c([2H])c1[2H]]

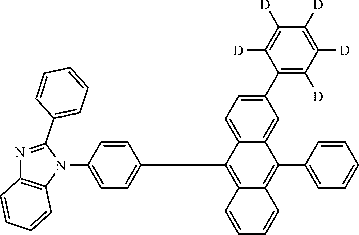 [SMILES: [2H]c1c([2H])c([2H])c(-c2ccc3c(-c4ccc(-n5c(-c6ccccc6)nc6ccccc65)cc4)c4ccccc4c(-c4ccccc4)c3c2)c([2H])c1[2H]]

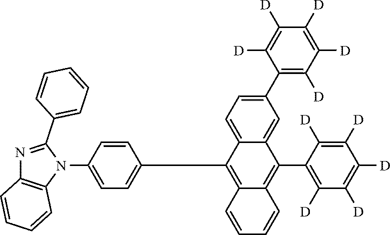 [SMILES: [2H]c1c([2H])c([2H])c(-c2ccc3c(-c4ccc(-n5c(-c6ccccc6)nc6ccccc65)cc4)c4ccccc4c(-c4c([2H])c([2H])c([2H])c([2H])c4[2H])c3c2)c([2H])c1[2H]]

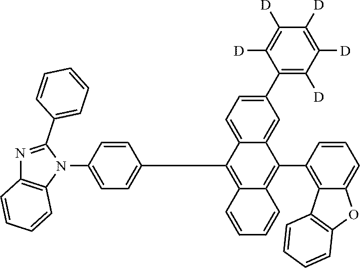 [SMILES: [2H]c1c([2H])c([2H])c(-c2ccc3c(-c4ccc(-n5c(-c6ccccc6)nc6ccccc65)cc4)c4ccccc4c(-c4cccc5oc6ccccc6c45)c3c2)c([2H])c1[2H]]

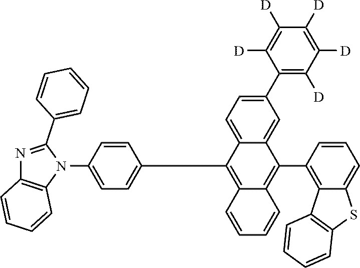 [SMILES: [2H]c1c([2H])c([2H])c(-c2ccc3c(-c4ccc(-n5c(-c6ccccc6)nc6ccccc65)cc4)c4ccccc4c(-c4cccc5sc6ccccc6c45)c3c2)c([2H])c1[2H]]

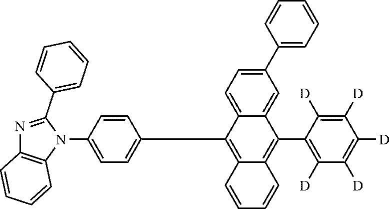 [SMILES: [2H]c1c([2H])c([2H])c(-c2c3ccccc3c(-c3ccc(-n4c(-c5ccccc5)nc5ccccc54)cc3)c3ccc(-c4ccccc4)cc23)c([2H])c1[2H]]

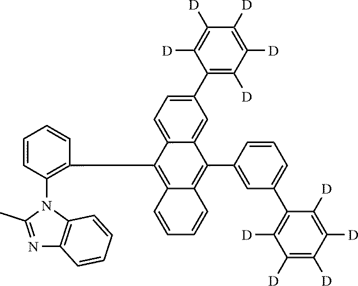 [SMILES: [2H]c1c([2H])c([2H])c(-c2cccc(-c3c4ccccc4c(-c4ccccc4-n4c(C)nc5ccccc54)c4ccc(-c5c([2H])c([2H])c([2H])c([2H])c5[2H])cc34)c2)c([2H])c1[2H]]

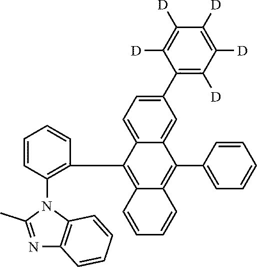 [SMILES: [2H]c1c([2H])c([2H])c(-c2ccc3c(-c4ccccc4-n4c(C)nc5ccccc54)c4ccccc4c(-c4ccccc4)c3c2)c([2H])c1[2H]]

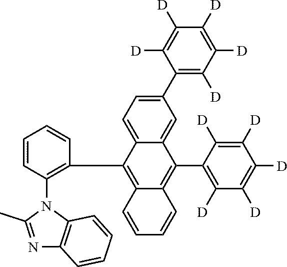 [SMILES: [2H]c1c([2H])c([2H])c(-c2ccc3c(-c4ccccc4-n4c(C)nc5ccccc54)c4ccccc4c(-c4c([2H])c([2H])c([2H])c([2H])c4[2H])c3c2)c([2H])c1[2H]]

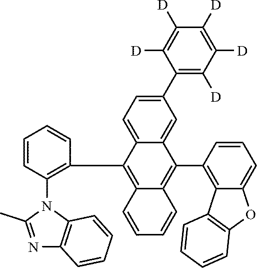 [SMILES: [2H]c1c([2H])c([2H])c(-c2ccc3c(-c4ccccc4-n4c(C)nc5ccccc54)c4ccccc4c(-c4cccc5oc6ccccc6c45)c3c2)c([2H])c1[2H]]

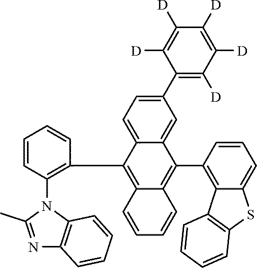 [SMILES: [2H]c1c([2H])c([2H])c(-c2ccc3c(-c4ccccc4-n4c(C)nc5ccccc54)c4ccccc4c(-c4cccc5sc6ccccc6c45)c3c2)c([2H])c1[2H]]

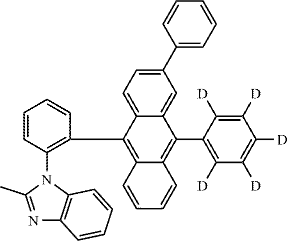 [SMILES: [2H]c1c([2H])c([2H])c(-c2c3ccccc3c(-c3ccccc3-n3c(C)nc4ccccc43)c3ccc(-c4ccccc4)cc23)c([2H])c1[2H]]

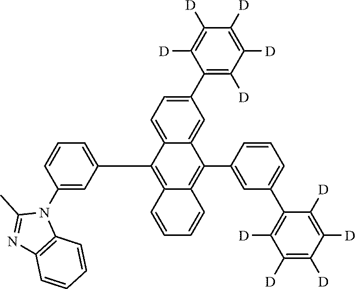 [SMILES: [2H]c1c([2H])c([2H])c(-c2cccc(-c3c4ccccc4c(-c4cccc(-n5c(C)nc6ccccc65)c4)c4ccc(-c5c([2H])c([2H])c([2H])c([2H])c5[2H])cc34)c2)c([2H])c1[2H]]

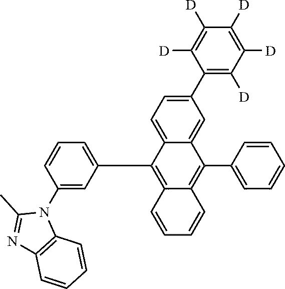 [SMILES: [2H]c1c([2H])c([2H])c(-c2ccc3c(-c4cccc(-n5c(C)nc6ccccc65)c4)c4ccccc4c(-c4ccccc4)c3c2)c([2H])c1[2H]]

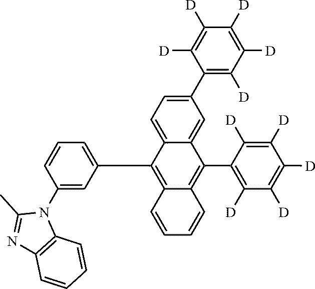 [SMILES: [2H]c1c([2H])c([2H])c(-c2ccc3c(-c4cccc(-n5c(C)nc6ccccc65)c4)c4ccccc4c(-c4c([2H])c([2H])c([2H])c([2H])c4[2H])c3c2)c([2H])c1[2H]]

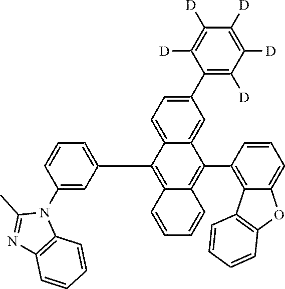 [SMILES: [2H]c1c([2H])c([2H])c(-c2ccc3c(-c4cccc(-n5c(C)nc6ccccc65)c4)c4ccccc4c(-c4cccc5oc6ccccc6c45)c3c2)c([2H])c1[2H]]

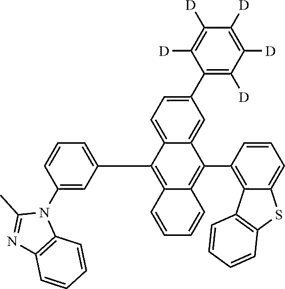 [SMILES: [2H]c1c([2H])c([2H])c(-c2ccc3c(-c4cccc(-n5c(C)nc6ccccc65)c4)c4ccccc4c(-c4cccc5sc6ccccc6c45)c3c2)c([2H])c1[2H]]

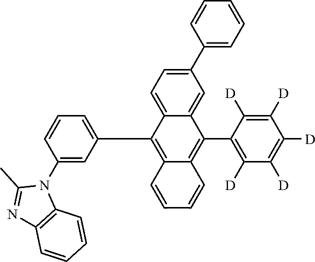 [SMILES: [2H]c1c([2H])c([2H])c(-c2c3ccccc3c(-c3cccc(-n4c(C)nc5ccccc54)c3)c3ccc(-c4ccccc4)cc23)c([2H])c1[2H]]

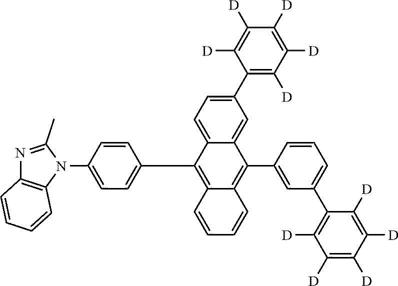 [SMILES: [2H]c1c([2H])c([2H])c(-c2cccc(-c3c4ccccc4c(-c4ccc(-n5c(C)nc6ccccc65)cc4)c4ccc(-c5c([2H])c([2H])c([2H])c([2H])c5[2H])cc34)c2)c([2H])c1[2H]]

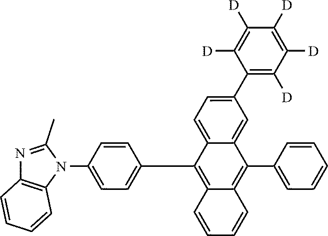 [SMILES: [2H]c1c([2H])c([2H])c(-c2ccc3c(-c4ccc(-n5c(C)nc6ccccc65)cc4)c4ccccc4c(-c4ccccc4)c3c2)c([2H])c1[2H]]

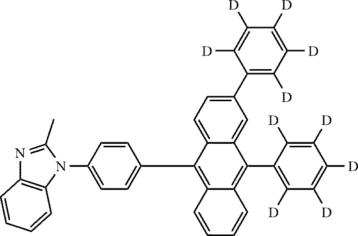 [SMILES: [2H]c1c([2H])c([2H])c(-c2ccc3c(-c4ccc(-n5c(C)nc6ccccc65)cc4)c4ccccc4c(-c4c([2H])c([2H])c([2H])c([2H])c4[2H])c3c2)c([2H])c1[2H]]

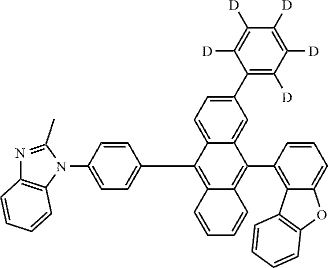 [SMILES: [2H]c1c([2H])c([2H])c(-c2ccc3c(-c4ccc(-n5c(C)nc6ccccc65)cc4)c4ccccc4c(-c4cccc5oc6ccccc6c45)c3c2)c([2H])c1[2H]]

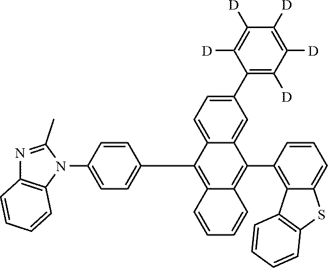 [SMILES: [2H]c1c([2H])c([2H])c(-c2ccc3c(-c4ccc(-n5c(C)nc6ccccc65)cc4)c4ccccc4c(-c4cccc5sc6ccccc6c45)c3c2)c([2H])c1[2H]]

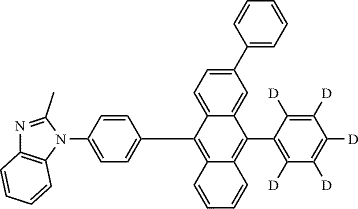 [SMILES: [2H]c1c([2H])c([2H])c(-c2c3ccccc3c(-c3ccc(-n4c(C)nc5ccccc54)cc3)c3ccc(-c4ccccc4)cc23)c([2H])c1[2H]]

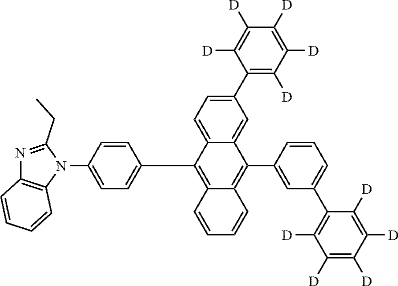 [SMILES: [2H]c1c([2H])c([2H])c(-c2cccc(-c3c4ccccc4c(-c4ccc(-n5c(CC)nc6ccccc65)cc4)c4ccc(-c5c([2H])c([2H])c([2H])c([2H])c5[2H])cc34)c2)c([2H])c1[2H]]

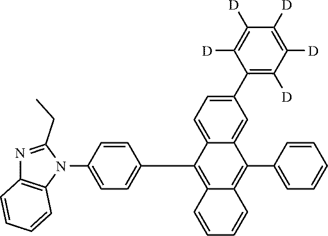 [SMILES: [2H]c1c([2H])c([2H])c(-c2ccc3c(-c4ccc(-n5c(CC)nc6ccccc65)cc4)c4ccccc4c(-c4ccccc4)c3c2)c([2H])c1[2H]]

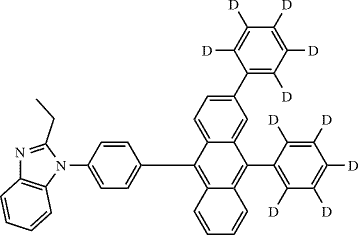 [SMILES: [2H]c1c([2H])c([2H])c(-c2ccc3c(-c4ccc(-n5c(CC)nc6ccccc65)cc4)c4ccccc4c(-c4c([2H])c([2H])c([2H])c([2H])c4[2H])c3c2)c([2H])c1[2H]]

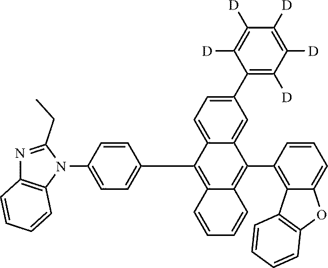 [SMILES: [2H]c1c([2H])c([2H])c(-c2ccc3c(-c4ccc(-n5c(CC)nc6ccccc65)cc4)c4ccccc4c(-c4cccc5oc6ccccc6c45)c3c2)c([2H])c1[2H]]

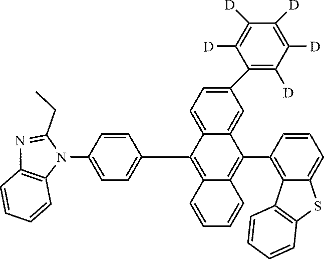 [SMILES: [2H]c1c([2H])c([2H])c(-c2ccc3c(-c4ccc(-n5c(CC)nc6ccccc65)cc4)c4ccccc4c(-c4cccc5sc6ccccc6c45)c3c2)c([2H])c1[2H]]

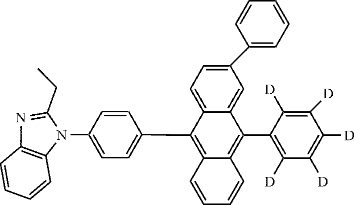 [SMILES: [2H]c1c([2H])c([2H])c(-c2c3ccccc3c(-c3ccc(-n4c(CC)nc5ccccc54)cc3)c3ccc(-c4ccccc4)cc23)c([2H])c1[2H]]

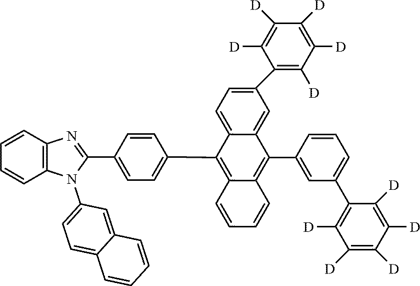 [SMILES: [2H]c1c([2H])c([2H])c(-c2cccc(-c3c4ccccc4c(-c4ccc(-c5nc6ccccc6n5-c5ccc6ccccc6c5)cc4)c4ccc(-c5c([2H])c([2H])c([2H])c([2H])c5[2H])cc34)c2)c([2H])c1[2H]]

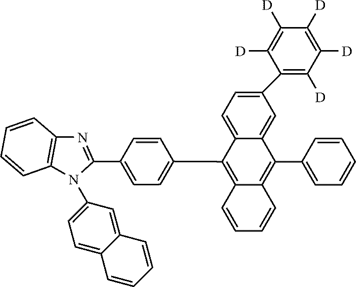 [SMILES: [2H]c1c([2H])c([2H])c(-c2ccc3c(-c4ccc(-c5nc6ccccc6n5-c5ccc6ccccc6c5)cc4)c4ccccc4c(-c4ccccc4)c3c2)c([2H])c1[2H]]

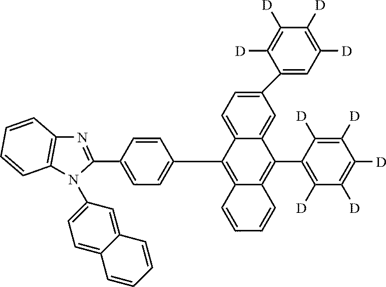 [SMILES: [2H]c1cc(-c2ccc3c(-c4ccc(-c5nc6ccccc6n5-c5ccc6ccccc6c5)cc4)c4ccccc4c(-c4c([2H])c([2H])c([2H])c([2H])c4[2H])c3c2)c([2H])c([2H])c1[2H]]

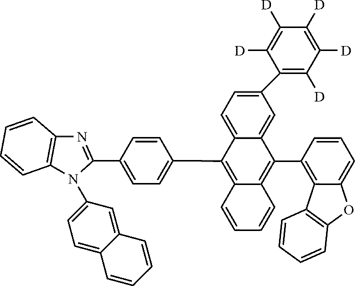 [SMILES: [2H]c1c([2H])c([2H])c(-c2ccc3c(-c4ccc(-c5nc6ccccc6n5-c5ccc6ccccc6c5)cc4)c4ccccc4c(-c4cccc5oc6ccccc6c45)c3c2)c([2H])c1[2H]]

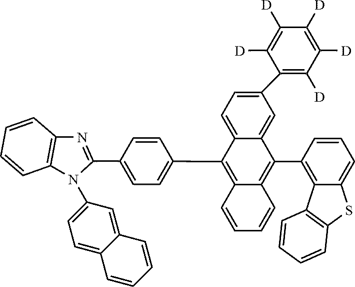 [SMILES: [2H]c1c([2H])c([2H])c(-c2ccc3c(-c4ccc(-c5nc6ccccc6n5-c5ccc6ccccc6c5)cc4)c4ccccc4c(-c4cccc5sc6ccccc6c45)c3c2)c([2H])c1[2H]]

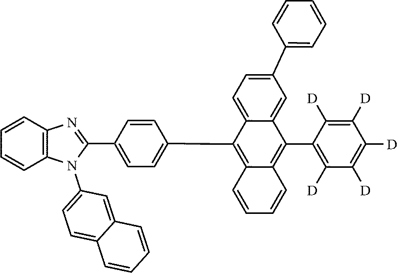 [SMILES: [2H]c1c([2H])c([2H])c(-c2c3ccccc3c(-c3ccc(-c4nc5ccccc5n4-c4ccc5ccccc5c4)cc3)c3ccc(-c4ccccc4)cc23)c([2H])c1[2H]]

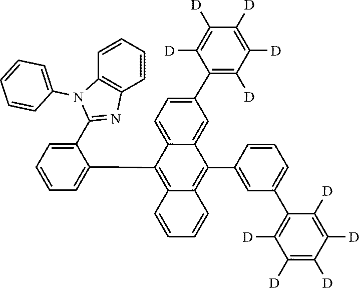 [SMILES: [2H]c1c([2H])c([2H])c(-c2cccc(-c3c4ccccc4c(-c4ccccc4-c4nc5ccccc5n4-c4ccccc4)c4ccc(-c5c([2H])c([2H])c([2H])c([2H])c5[2H])cc34)c2)c([2H])c1[2H]]

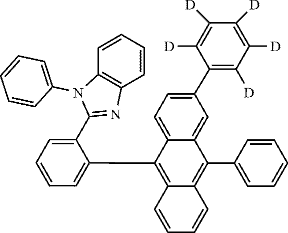 [SMILES: [2H]c1c([2H])c([2H])c(-c2ccc3c(-c4ccccc4-c4nc5ccccc5n4-c4ccccc4)c4ccccc4c(-c4ccccc4)c3c2)c([2H])c1[2H]]

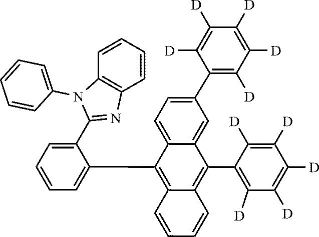 [SMILES: [2H]c1c([2H])c([2H])c(-c2ccc3c(-c4ccccc4-c4nc5ccccc5n4-c4ccccc4)c4ccccc4c(-c4c([2H])c([2H])c([2H])c([2H])c4[2H])c3c2)c([2H])c1[2H]]

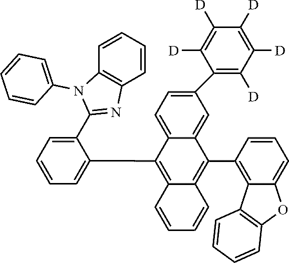 [SMILES: [2H]c1c([2H])c([2H])c(-c2ccc3c(-c4ccccc4-c4nc5ccccc5n4-c4ccccc4)c4ccccc4c(-c4cccc5oc6ccccc6c45)c3c2)c([2H])c1[2H]]

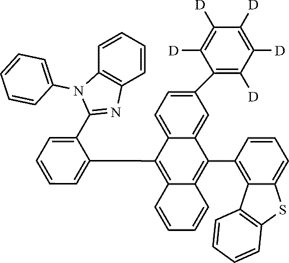 [SMILES: [2H]c1c([2H])c([2H])c(-c2ccc3c(-c4ccccc4-c4nc5ccccc5n4-c4ccccc4)c4ccccc4c(-c4cccc5sc6ccccc6c45)c3c2)c([2H])c1[2H]]

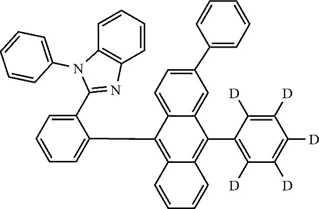 [SMILES: [2H]c1c([2H])c([2H])c(-c2c3ccccc3c(-c3ccccc3-c3nc4ccccc4n3-c3ccccc3)c3ccc(-c4ccccc4)cc23)c([2H])c1[2H]]

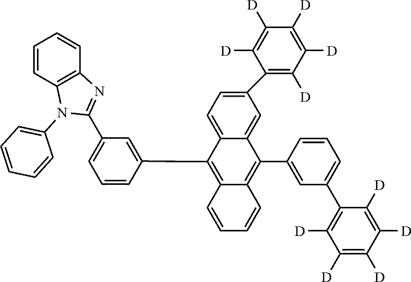 [SMILES: [2H]c1c([2H])c([2H])c(-c2cccc(-c3c4ccccc4c(-c4cccc(-c5nc6ccccc6n5-c5ccccc5)c4)c4ccc(-c5c([2H])c([2H])c([2H])c([2H])c5[2H])cc34)c2)c([2H])c1[2H]]

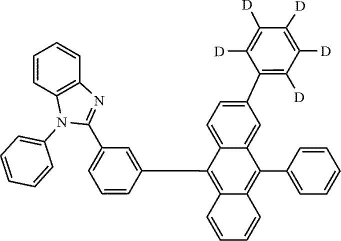 [SMILES: [2H]c1c([2H])c([2H])c(-c2ccc3c(-c4cccc(-c5nc6ccccc6n5-c5ccccc5)c4)c4ccccc4c(-c4ccccc4)c3c2)c([2H])c1[2H]]

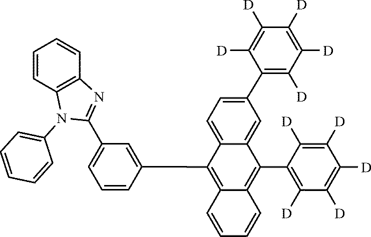 [SMILES: [2H]c1c([2H])c([2H])c(-c2ccc3c(-c4cccc(-c5nc6ccccc6n5-c5ccccc5)c4)c4ccccc4c(-c4c([2H])c([2H])c([2H])c([2H])c4[2H])c3c2)c([2H])c1[2H]]

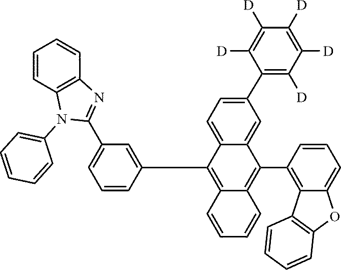 [SMILES: [2H]c1c([2H])c([2H])c(-c2ccc3c(-c4cccc(-c5nc6ccccc6n5-c5ccccc5)c4)c4ccccc4c(-c4cccc5oc6ccccc6c45)c3c2)c([2H])c1[2H]]

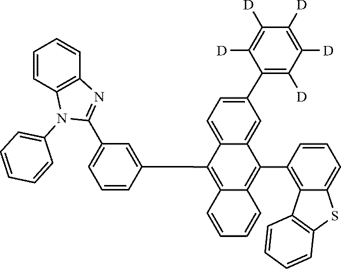 [SMILES: [2H]c1c([2H])c([2H])c(-c2ccc3c(-c4cccc(-c5nc6ccccc6n5-c5ccccc5)c4)c4ccccc4c(-c4cccc5sc6ccccc6c45)c3c2)c([2H])c1[2H]]

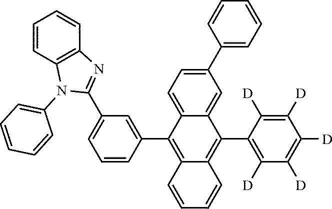 [SMILES: [2H]c1c([2H])c([2H])c(-c2c3ccccc3c(-c3cccc(-c4nc5ccccc5n4-c4ccccc4)c3)c3ccc(-c4ccccc4)cc23)c([2H])c1[2H]]

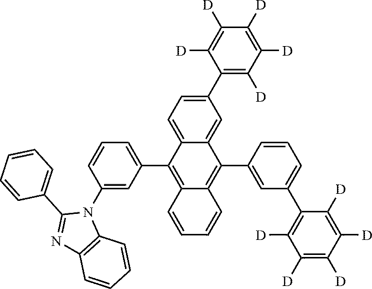 [SMILES: [2H]c1c([2H])c([2H])c(-c2cccc(-c3c4ccccc4c(-c4cccc(-n5c(-c6ccccc6)nc6ccccc65)c4)c4ccc(-c5c([2H])c([2H])c([2H])c([2H])c5[2H])cc34)c2)c([2H])c1[2H]]

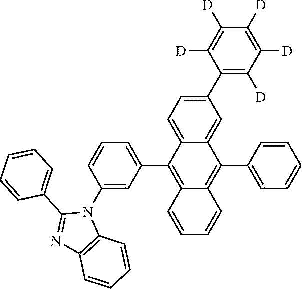 [SMILES: [2H]c1c([2H])c([2H])c(-c2ccc3c(-c4cccc(-n5c(-c6ccccc6)nc6ccccc65)c4)c4ccccc4c(-c4ccccc4)c3c2)c([2H])c1[2H]]

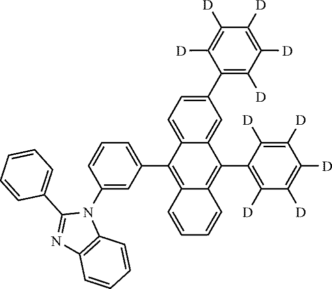 [SMILES: [2H]c1c([2H])c([2H])c(-c2ccc3c(-c4cccc(-n5c(-c6ccccc6)nc6ccccc65)c4)c4ccccc4c(-c4c([2H])c([2H])c([2H])c([2H])c4[2H])c3c2)c([2H])c1[2H]]

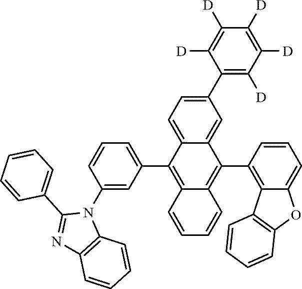 [SMILES: [2H]c1c([2H])c([2H])c(-c2ccc3c(-c4cccc(-n5c(-c6ccccc6)nc6ccccc65)c4)c4ccccc4c(-c4cccc5oc6ccccc6c45)c3c2)c([2H])c1[2H]]

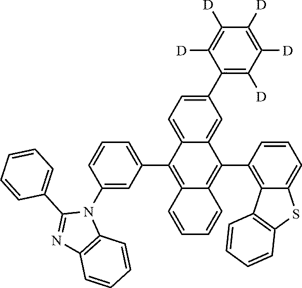 [SMILES: [2H]c1c([2H])c([2H])c(-c2ccc3c(-c4cccc(-n5c(-c6ccccc6)nc6ccccc65)c4)c4ccccc4c(-c4cccc5sc6ccccc6c45)c3c2)c([2H])c1[2H]]

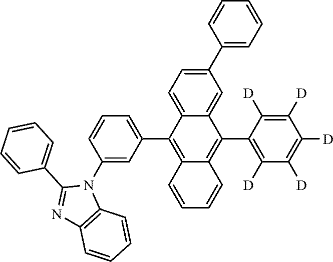 [SMILES: [2H]c1c([2H])c([2H])c(-c2c3ccccc3c(-c3cccc(-n4c(-c5ccccc5)nc5ccccc54)c3)c3ccc(-c4ccccc4)cc23)c([2H])c1[2H]]

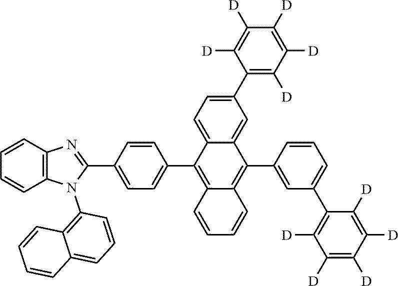 [SMILES: [2H]c1c([2H])c([2H])c(-c2cccc(-c3c4ccccc4c(-c4ccc(-c5nc6ccccc6n5-c5cccc6ccccc56)cc4)c4ccc(-c5c([2H])c([2H])c([2H])c([2H])c5[2H])cc34)c2)c([2H])c1[2H]]

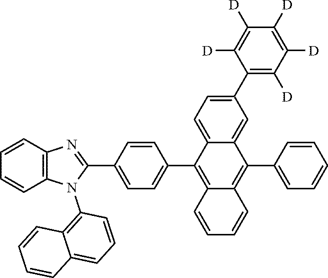 [SMILES: [2H]c1c([2H])c([2H])c(-c2ccc3c(-c4ccc(-c5nc6ccccc6n5-c5cccc6ccccc56)cc4)c4ccccc4c(-c4ccccc4)c3c2)c([2H])c1[2H]]

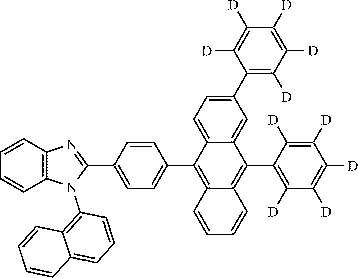 [SMILES: [2H]c1c([2H])c([2H])c(-c2ccc3c(-c4ccc(-c5nc6ccccc6n5-c5cccc6ccccc56)cc4)c4ccccc4c(-c4c([2H])c([2H])c([2H])c([2H])c4[2H])c3c2)c([2H])c1[2H]]

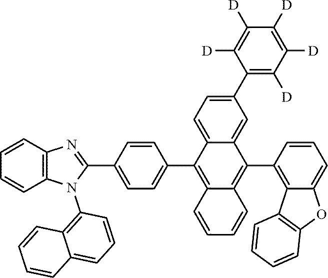 [SMILES: [2H]c1c([2H])c([2H])c(-c2ccc3c(-c4ccc(-c5nc6ccccc6n5-c5cccc6ccccc56)cc4)c4ccccc4c(-c4cccc5oc6ccccc6c45)c3c2)c([2H])c1[2H]]

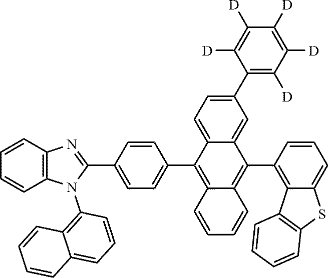 [SMILES: [2H]c1c([2H])c([2H])c(-c2ccc3c(-c4ccc(-c5nc6ccccc6n5-c5cccc6ccccc56)cc4)c4ccccc4c(-c4cccc5sc6ccccc6c45)c3c2)c([2H])c1[2H]]

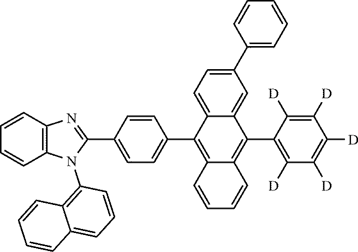 [SMILES: [2H]c1c([2H])c([2H])c(-c2c3ccccc3c(-c3ccc(-c4nc5ccccc5n4-c4cccc5ccccc45)cc3)c3ccc(-c4ccccc4)cc23)c([2H])c1[2H]]

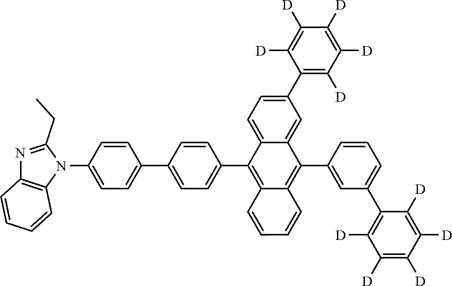 [SMILES: [2H]c1c([2H])c([2H])c(-c2cccc(-c3c4ccccc4c(-c4ccc(-c5ccc(-n6c(CC)nc7ccccc76)cc5)cc4)c4ccc(-c5c([2H])c([2H])c([2H])c([2H])c5[2H])cc34)c2)c([2H])c1[2H]]

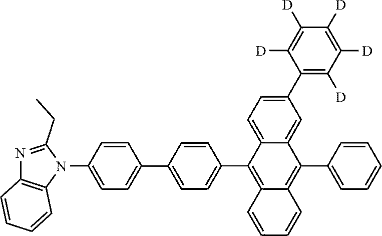 [SMILES: [2H]c1c([2H])c([2H])c(-c2ccc3c(-c4ccc(-c5ccc(-n6c(CC)nc7ccccc76)cc5)cc4)c4ccccc4c(-c4ccccc4)c3c2)c([2H])c1[2H]]

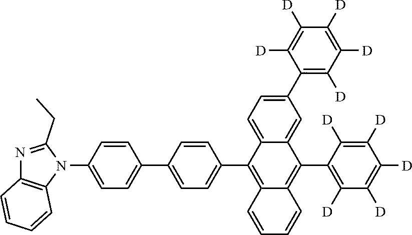 [SMILES: [2H]c1c([2H])c([2H])c(-c2ccc3c(-c4ccc(-c5ccc(-n6c(CC)nc7ccccc76)cc5)cc4)c4ccccc4c(-c4c([2H])c([2H])c([2H])c([2H])c4[2H])c3c2)c([2H])c1[2H]]

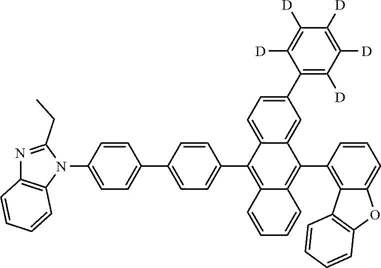 [SMILES: [2H]c1c([2H])c([2H])c(-c2ccc3c(-c4ccc(-c5ccc(-n6c(CC)nc7ccccc76)cc5)cc4)c4ccccc4c(-c4cccc5oc6ccccc6c45)c3c2)c([2H])c1[2H]]

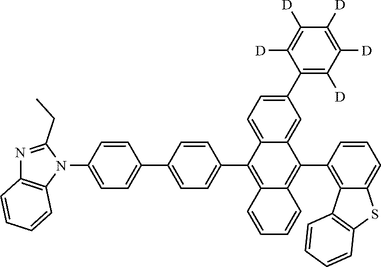 [SMILES: [2H]c1c([2H])c([2H])c(-c2ccc3c(-c4ccc(-c5ccc(-n6c(CC)nc7ccccc76)cc5)cc4)c4ccccc4c(-c4cccc5sc6ccccc6c45)c3c2)c([2H])c1[2H]]

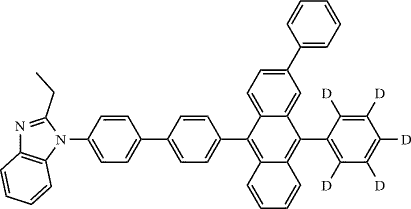 [SMILES: [2H]c1c([2H])c([2H])c(-c2c3ccccc3c(-c3ccc(-c4ccc(-n5c(CC)nc6ccccc65)cc4)cc3)c3ccc(-c4ccccc4)cc23)c([2H])c1[2H]]

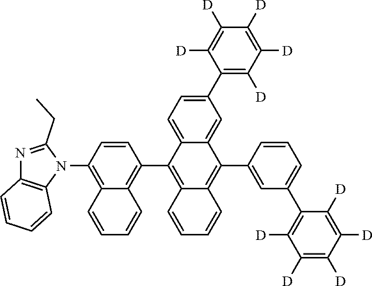 [SMILES: [2H]c1c([2H])c([2H])c(-c2cccc(-c3c4ccccc4c(-c4ccc(-n5c(CC)nc6ccccc65)c5ccccc45)c4ccc(-c5c([2H])c([2H])c([2H])c([2H])c5[2H])cc34)c2)c([2H])c1[2H]]

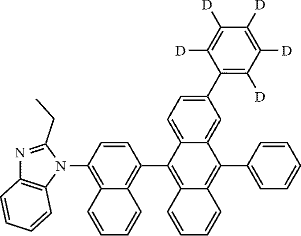 [SMILES: [2H]c1c([2H])c([2H])c(-c2ccc3c(-c4ccc(-n5c(CC)nc6ccccc65)c5ccccc45)c4ccccc4c(-c4ccccc4)c3c2)c([2H])c1[2H]]

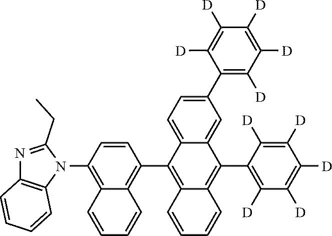 [SMILES: [2H]c1c([2H])c([2H])c(-c2ccc3c(-c4ccc(-n5c(CC)nc6ccccc65)c5ccccc45)c4ccccc4c(-c4c([2H])c([2H])c([2H])c([2H])c4[2H])c3c2)c([2H])c1[2H]]

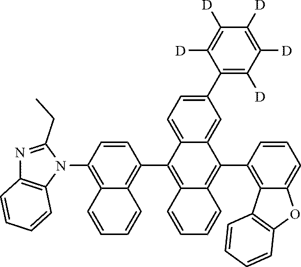 [SMILES: [2H]c1c([2H])c([2H])c(-c2ccc3c(-c4ccc(-n5c(CC)nc6ccccc65)c5ccccc45)c4ccccc4c(-c4cccc5oc6ccccc6c45)c3c2)c([2H])c1[2H]]

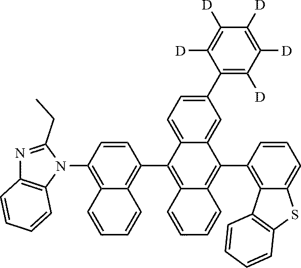 [SMILES: [2H]c1c([2H])c([2H])c(-c2ccc3c(-c4ccc(-n5c(CC)nc6ccccc65)c5ccccc45)c4ccccc4c(-c4cccc5sc6ccccc6c45)c3c2)c([2H])c1[2H]]

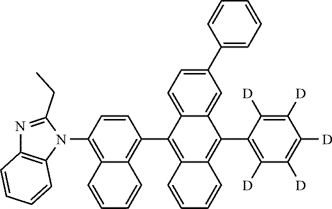 [SMILES: [2H]c1c([2H])c([2H])c(-c2c3ccccc3c(-c3ccc(-n4c(CC)nc5ccccc54)c4ccccc34)c3ccc(-c4ccccc4)cc23)c([2H])c1[2H]]

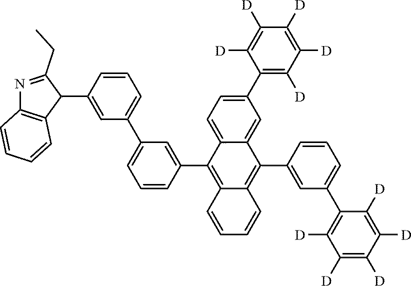 [SMILES: [2H]c1c([2H])c([2H])c(-c2cccc(-c3c4ccccc4c(-c4cccc(-c5cccc(C6C(CC)=Nc7ccccc76)c5)c4)c4ccc(-c5c([2H])c([2H])c([2H])c([2H])c5[2H])cc34)c2)c([2H])c1[2H]]

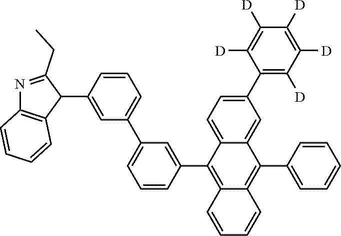 [SMILES: [2H]c1c([2H])c([2H])c(-c2ccc3c(-c4cccc(-c5cccc(C6C(CC)=Nc7ccccc76)c5)c4)c4ccccc4c(-c4ccccc4)c3c2)c([2H])c1[2H]]

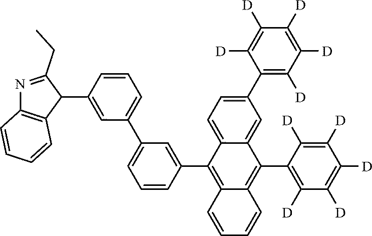 [SMILES: [2H]c1c([2H])c([2H])c(-c2ccc3c(-c4cccc(-c5cccc(C6C(CC)=Nc7ccccc76)c5)c4)c4ccccc4c(-c4c([2H])c([2H])c([2H])c([2H])c4[2H])c3c2)c([2H])c1[2H]]

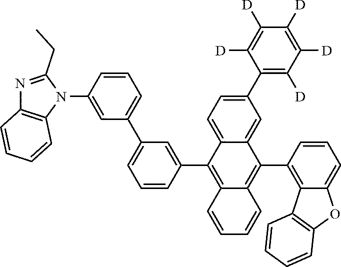 [SMILES: [2H]c1c([2H])c([2H])c(-c2ccc3c(-c4cccc(-c5cccc(-n6c(CC)nc7ccccc76)c5)c4)c4ccccc4c(-c4cccc5oc6ccccc6c45)c3c2)c([2H])c1[2H]]

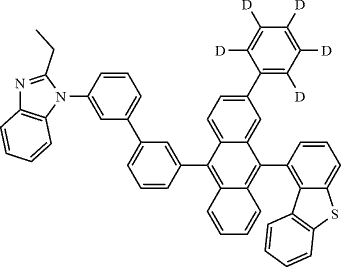 [SMILES: [2H]c1c([2H])c([2H])c(-c2ccc3c(-c4cccc(-c5cccc(-n6c(CC)nc7ccccc76)c5)c4)c4ccccc4c(-c4cccc5sc6ccccc6c45)c3c2)c([2H])c1[2H]]

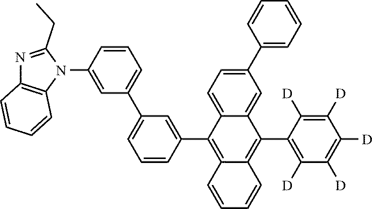 [SMILES: [2H]c1c([2H])c([2H])c(-c2c3ccccc3c(-c3cccc(-c4cccc(-n5c(CC)nc6ccccc65)c4)c3)c3ccc(-c4ccccc4)cc23)c([2H])c1[2H]]

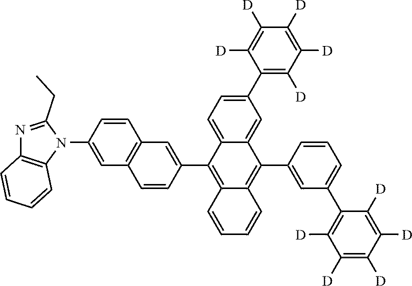 [SMILES: [2H]c1c([2H])c([2H])c(-c2cccc(-c3c4ccccc4c(-c4ccc5cc(-n6c(CC)nc7ccccc76)ccc5c4)c4ccc(-c5c([2H])c([2H])c([2H])c([2H])c5[2H])cc34)c2)c([2H])c1[2H]]

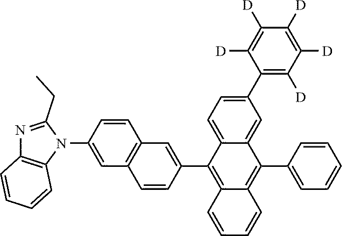 [SMILES: [2H]c1c([2H])c([2H])c(-c2ccc3c(-c4ccc5cc(-n6c(CC)nc7ccccc76)ccc5c4)c4ccccc4c(-c4ccccc4)c3c2)c([2H])c1[2H]]

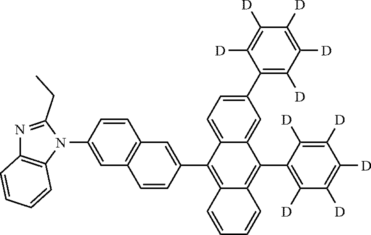 [SMILES: [2H]c1c([2H])c([2H])c(-c2ccc3c(-c4ccc5cc(-n6c(CC)nc7ccccc76)ccc5c4)c4ccccc4c(-c4c([2H])c([2H])c([2H])c([2H])c4[2H])c3c2)c([2H])c1[2H]]

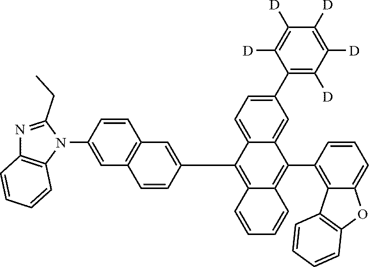 [SMILES: [2H]c1c([2H])c([2H])c(-c2ccc3c(-c4ccc5cc(-n6c(CC)nc7ccccc76)ccc5c4)c4ccccc4c(-c4cccc5oc6ccccc6c45)c3c2)c([2H])c1[2H]]

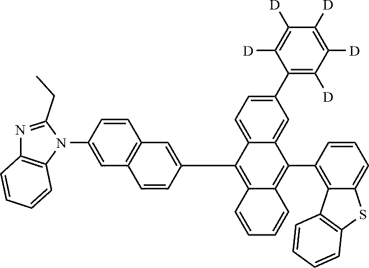 [SMILES: [2H]c1c([2H])c([2H])c(-c2ccc3c(-c4ccc5cc(-n6c(CC)nc7ccccc76)ccc5c4)c4ccccc4c(-c4cccc5sc6ccccc6c45)c3c2)c([2H])c1[2H]]

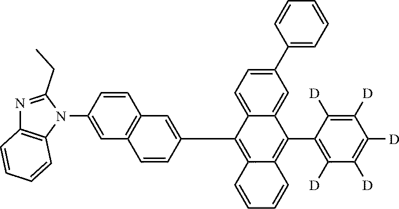 [SMILES: [2H]c1c([2H])c([2H])c(-c2c3ccccc3c(-c3ccc4cc(-n5c(CC)nc6ccccc65)ccc4c3)c3ccc(-c4ccccc4)cc23)c([2H])c1[2H]]

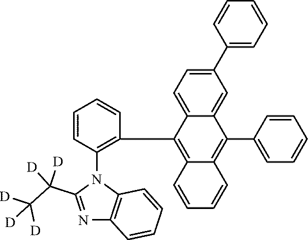 [SMILES: [2H]C([2H])([2H])C([2H])([2H])c1nc2ccccc2n1-c1ccccc1-c1c2ccccc2c(-c2ccccc2)c2cc(-c3ccccc3)ccc12]

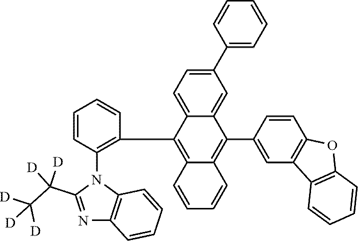 [SMILES: [2H]C([2H])([2H])C([2H])([2H])c1nc2ccccc2n1-c1ccccc1-c1c2ccccc2c(-c2ccc3oc4ccccc4c3c2)c2cc(-c3ccccc3)ccc12]

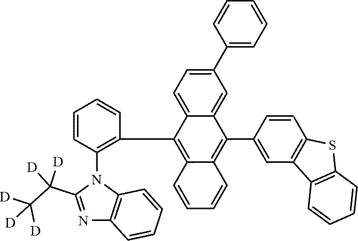 [SMILES: [2H]C([2H])([2H])C([2H])([2H])c1nc2ccccc2n1-c1ccccc1-c1c2ccccc2c(-c2ccc3sc4ccccc4c3c2)c2cc(-c3ccccc3)ccc12]

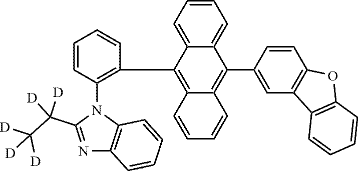 [SMILES: [2H]C([2H])([2H])C([2H])([2H])c1nc2ccccc2n1-c1ccccc1-c1c2ccccc2c(-c2ccc3oc4ccccc4c3c2)c2ccccc12]

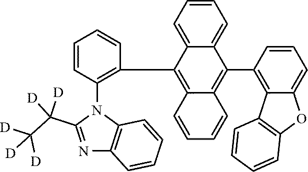 [SMILES: [2H]C([2H])([2H])C([2H])([2H])c1nc2ccccc2n1-c1ccccc1-c1c2ccccc2c(-c2cccc3oc4ccccc4c23)c2ccccc12]

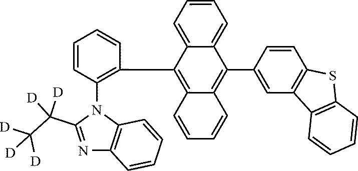 [SMILES: [2H]C([2H])([2H])C([2H])([2H])c1nc2ccccc2n1-c1ccccc1-c1c2ccccc2c(-c2ccc3sc4ccccc4c3c2)c2ccccc12]

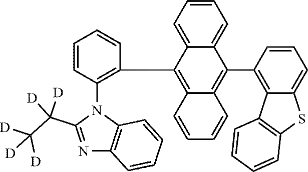 [SMILES: [2H]C([2H])([2H])C([2H])([2H])c1nc2ccccc2n1-c1ccccc1-c1c2ccccc2c(-c2cccc3sc4ccccc4c23)c2ccccc12]

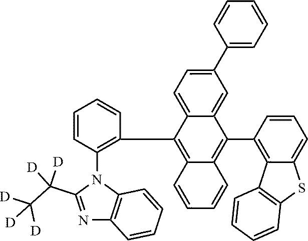 [SMILES: [2H]C([2H])([2H])C([2H])([2H])c1nc2ccccc2n1-c1ccccc1-c1c2ccccc2c(-c2cccc3sc4ccccc4c23)c2cc(-c3ccccc3)ccc12]

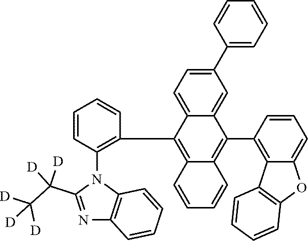 [SMILES: [2H]C([2H])([2H])C([2H])([2H])c1nc2ccccc2n1-c1ccccc1-c1c2ccccc2c(-c2cccc3oc4ccccc4c23)c2cc(-c3ccccc3)ccc12]

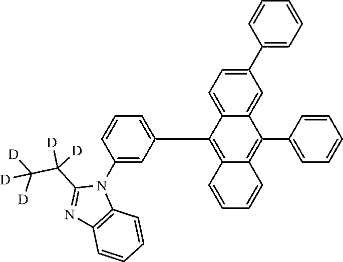 [SMILES: [2H]C([2H])([2H])C([2H])([2H])c1nc2ccccc2n1-c1cccc(-c2c3ccccc3c(-c3ccccc3)c3cc(-c4ccccc4)ccc23)c1]

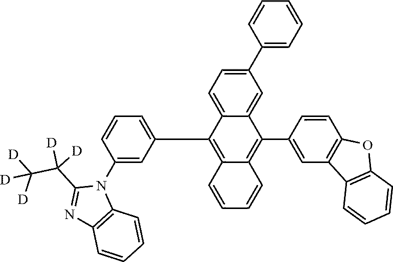 [SMILES: [2H]C([2H])([2H])C([2H])([2H])c1nc2ccccc2n1-c1cccc(-c2c3ccccc3c(-c3ccc4oc5ccccc5c4c3)c3cc(-c4ccccc4)ccc23)c1]

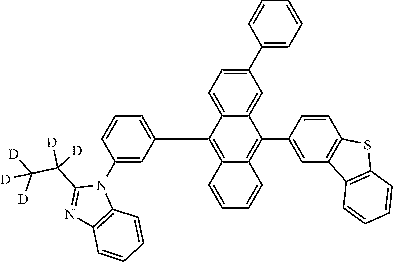 [SMILES: [2H]C([2H])([2H])C([2H])([2H])c1nc2ccccc2n1-c1cccc(-c2c3ccccc3c(-c3ccc4sc5ccccc5c4c3)c3cc(-c4ccccc4)ccc23)c1]

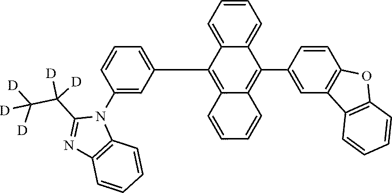 [SMILES: [2H]C([2H])([2H])C([2H])([2H])c1nc2ccccc2n1-c1cccc(-c2c3ccccc3c(-c3ccc4oc5ccccc5c4c3)c3ccccc23)c1]

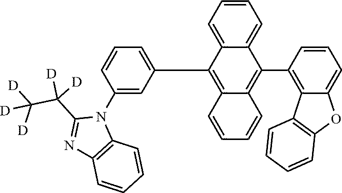 [SMILES: [2H]C([2H])([2H])C([2H])([2H])c1nc2ccccc2n1-c1cccc(-c2c3ccccc3c(-c3cccc4oc5ccccc5c34)c3ccccc23)c1]

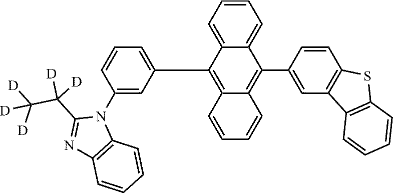 [SMILES: [2H]C([2H])([2H])C([2H])([2H])c1nc2ccccc2n1-c1cccc(-c2c3ccccc3c(-c3ccc4sc5ccccc5c4c3)c3ccccc23)c1]

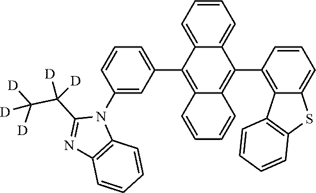 [SMILES: [2H]C([2H])([2H])C([2H])([2H])c1nc2ccccc2n1-c1cccc(-c2c3ccccc3c(-c3cccc4sc5ccccc5c34)c3ccccc23)c1]

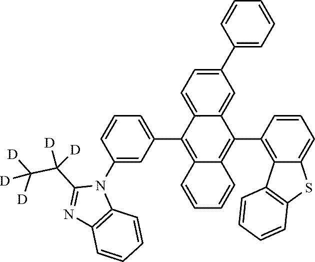 [SMILES: [2H]C([2H])([2H])C([2H])([2H])c1nc2ccccc2n1-c1cccc(-c2c3ccccc3c(-c3cccc4sc5ccccc5c34)c3cc(-c4ccccc4)ccc23)c1]

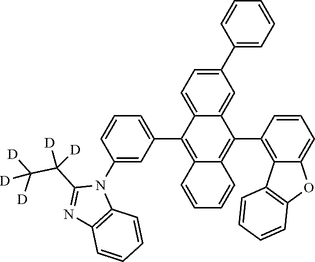 [SMILES: [2H]C([2H])([2H])C([2H])([2H])c1nc2ccccc2n1-c1cccc(-c2c3ccccc3c(-c3cccc4oc5ccccc5c34)c3cc(-c4ccccc4)ccc23)c1]

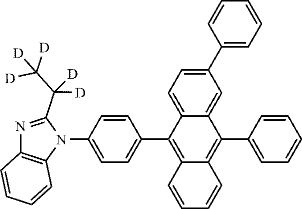 [SMILES: [2H]C([2H])([2H])C([2H])([2H])c1nc2ccccc2n1-c1ccc(-c2c3ccccc3c(-c3ccccc3)c3cc(-c4ccccc4)ccc23)cc1]

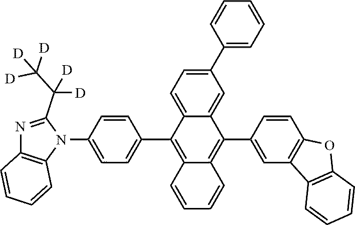 [SMILES: [2H]C([2H])([2H])C([2H])([2H])c1nc2ccccc2n1-c1ccc(-c2c3ccccc3c(-c3ccc4oc5ccccc5c4c3)c3cc(-c4ccccc4)ccc23)cc1]

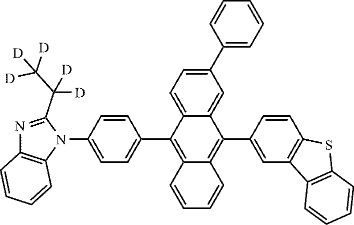 [SMILES: [2H]C([2H])([2H])C([2H])([2H])c1nc2ccccc2n1-c1ccc(-c2c3ccccc3c(-c3ccc4sc5ccccc5c4c3)c3cc(-c4ccccc4)ccc23)cc1]

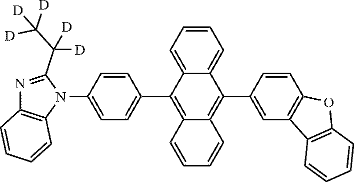 [SMILES: [2H]C([2H])([2H])C([2H])([2H])c1nc2ccccc2n1-c1ccc(-c2c3ccccc3c(-c3ccc4oc5ccccc5c4c3)c3ccccc23)cc1]

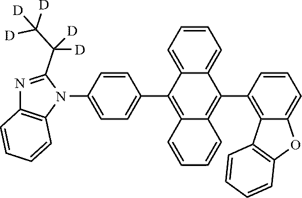 [SMILES: [2H]C([2H])([2H])C([2H])([2H])c1nc2ccccc2n1-c1ccc(-c2c3ccccc3c(-c3cccc4oc5ccccc5c34)c3ccccc23)cc1]

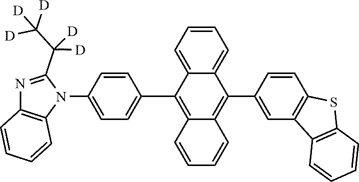 [SMILES: [2H]C([2H])([2H])C([2H])([2H])c1nc2ccccc2n1-c1ccc(-c2c3ccccc3c(-c3ccc4sc5ccccc5c4c3)c3ccccc23)cc1]

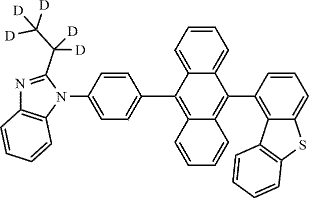 [SMILES: [2H]C([2H])([2H])C([2H])([2H])c1nc2ccccc2n1-c1ccc(-c2c3ccccc3c(-c3cccc4sc5ccccc5c34)c3ccccc23)cc1]

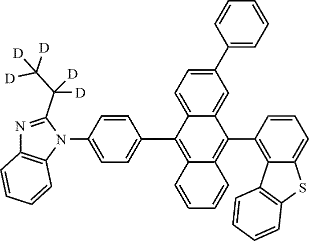 [SMILES: [2H]C([2H])([2H])C([2H])([2H])c1nc2ccccc2n1-c1ccc(-c2c3ccccc3c(-c3cccc4sc5ccccc5c34)c3cc(-c4ccccc4)ccc23)cc1]

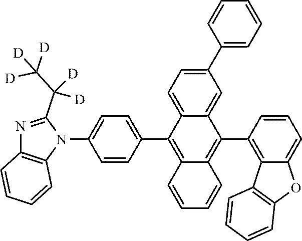 [SMILES: [2H]C([2H])([2H])C([2H])([2H])c1nc2ccccc2n1-c1ccc(-c2c3ccccc3c(-c3cccc4oc5ccccc5c34)c3cc(-c4ccccc4)ccc23)cc1]

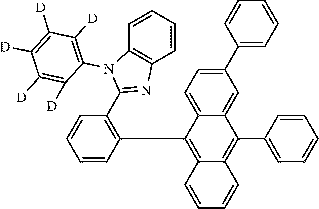 [SMILES: [2H]c1c([2H])c([2H])c(-n2c(-c3ccccc3-c3c4ccccc4c(-c4ccccc4)c4cc(-c5ccccc5)ccc34)nc3ccccc32)c([2H])c1[2H]]

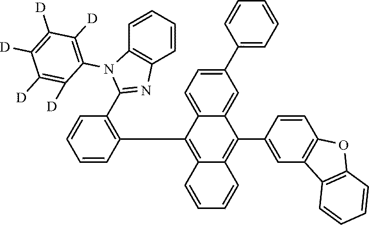 [SMILES: [2H]c1c([2H])c([2H])c(-n2c(-c3ccccc3-c3c4ccccc4c(-c4ccc5oc6ccccc6c5c4)c4cc(-c5ccccc5)ccc34)nc3ccccc32)c([2H])c1[2H]]

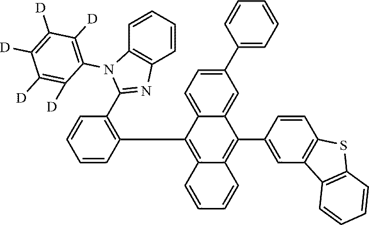 [SMILES: [2H]c1c([2H])c([2H])c(-n2c(-c3ccccc3-c3c4ccccc4c(-c4ccc5sc6ccccc6c5c4)c4cc(-c5ccccc5)ccc34)nc3ccccc32)c([2H])c1[2H]]

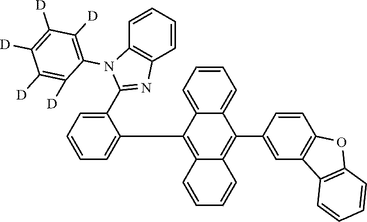 [SMILES: [2H]c1c([2H])c([2H])c(-n2c(-c3ccccc3-c3c4ccccc4c(-c4ccc5oc6ccccc6c5c4)c4ccccc34)nc3ccccc32)c([2H])c1[2H]]

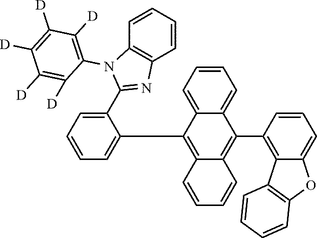 [SMILES: [2H]c1c([2H])c([2H])c(-n2c(-c3ccccc3-c3c4ccccc4c(-c4cccc5oc6ccccc6c45)c4ccccc34)nc3ccccc32)c([2H])c1[2H]]

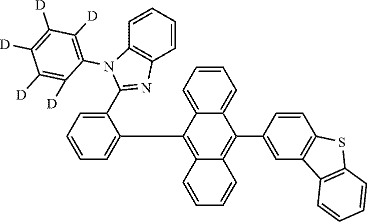 [SMILES: [2H]c1c([2H])c([2H])c(-n2c(-c3ccccc3-c3c4ccccc4c(-c4ccc5sc6ccccc6c5c4)c4ccccc34)nc3ccccc32)c([2H])c1[2H]]

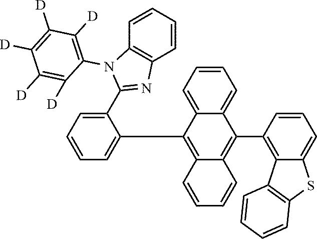 [SMILES: [2H]c1c([2H])c([2H])c(-n2c(-c3ccccc3-c3c4ccccc4c(-c4cccc5sc6ccccc6c45)c4ccccc34)nc3ccccc32)c([2H])c1[2H]]

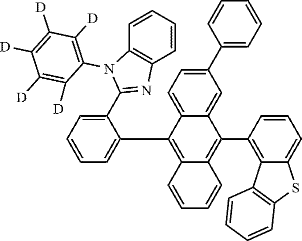 [SMILES: [2H]c1c([2H])c([2H])c(-n2c(-c3ccccc3-c3c4ccccc4c(-c4cccc5sc6ccccc6c45)c4cc(-c5ccccc5)ccc34)nc3ccccc32)c([2H])c1[2H]]

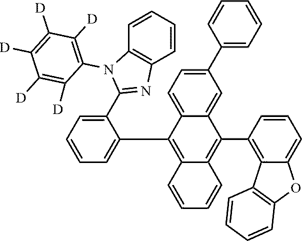 [SMILES: [2H]c1c([2H])c([2H])c(-n2c(-c3ccccc3-c3c4ccccc4c(-c4cccc5oc6ccccc6c45)c4cc(-c5ccccc5)ccc34)nc3ccccc32)c([2H])c1[2H]]

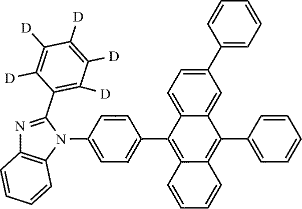 [SMILES: [2H]c1c([2H])c([2H])c(-c2nc3ccccc3n2-c2ccc(-c3c4ccccc4c(-c4ccccc4)c4cc(-c5ccccc5)ccc34)cc2)c([2H])c1[2H]]

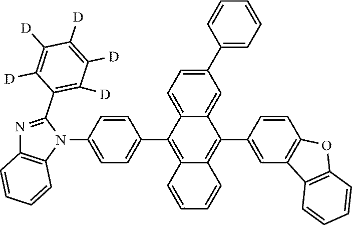 [SMILES: [2H]c1c([2H])c([2H])c(-c2nc3ccccc3n2-c2ccc(-c3c4ccccc4c(-c4ccc5oc6ccccc6c5c4)c4cc(-c5ccccc5)ccc34)cc2)c([2H])c1[2H]]

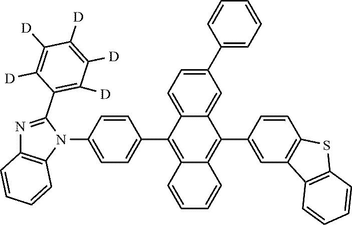 [SMILES: [2H]c1c([2H])c([2H])c(-c2nc3ccccc3n2-c2ccc(-c3c4ccccc4c(-c4ccc5sc6ccccc6c5c4)c4cc(-c5ccccc5)ccc34)cc2)c([2H])c1[2H]]

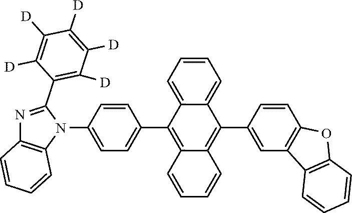 [SMILES: [2H]c1c([2H])c([2H])c(-c2nc3ccccc3n2-c2ccc(-c3c4ccccc4c(-c4ccc5oc6ccccc6c5c4)c4ccccc34)cc2)c([2H])c1[2H]]

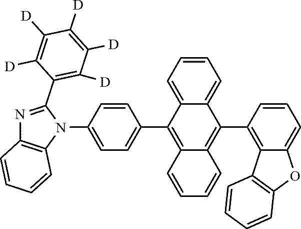 [SMILES: [2H]c1c([2H])c([2H])c(-c2nc3ccccc3n2-c2ccc(-c3c4ccccc4c(-c4cccc5oc6ccccc6c45)c4ccccc34)cc2)c([2H])c1[2H]]

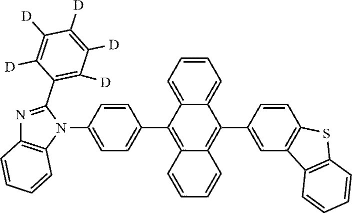 [SMILES: [2H]c1c([2H])c([2H])c(-c2nc3ccccc3n2-c2ccc(-c3c4ccccc4c(-c4ccc5sc6ccccc6c5c4)c4ccccc34)cc2)c([2H])c1[2H]]

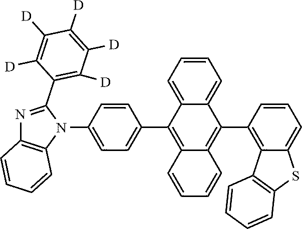 [SMILES: [2H]c1c([2H])c([2H])c(-c2nc3ccccc3n2-c2ccc(-c3c4ccccc4c(-c4cccc5sc6ccccc6c45)c4ccccc34)cc2)c([2H])c1[2H]]

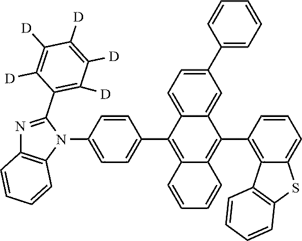 [SMILES: [2H]c1c([2H])c([2H])c(-c2nc3ccccc3n2-c2ccc(-c3c4ccccc4c(-c4cccc5sc6ccccc6c45)c4cc(-c5ccccc5)ccc34)cc2)c([2H])c1[2H]]

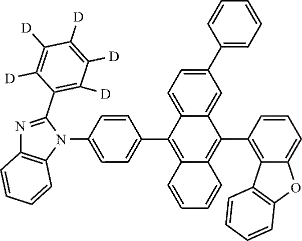 [SMILES: [2H]c1c([2H])c([2H])c(-c2nc3ccccc3n2-c2ccc(-c3c4ccccc4c(-c4cccc5oc6ccccc6c45)c4cc(-c5ccccc5)ccc34)cc2)c([2H])c1[2H]]

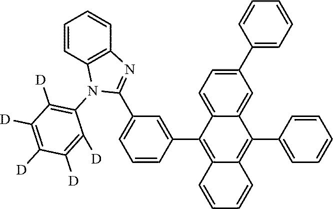 [SMILES: [2H]c1c([2H])c([2H])c(-n2c(-c3cccc(-c4c5ccccc5c(-c5ccccc5)c5cc(-c6ccccc6)ccc45)c3)nc3ccccc32)c([2H])c1[2H]]

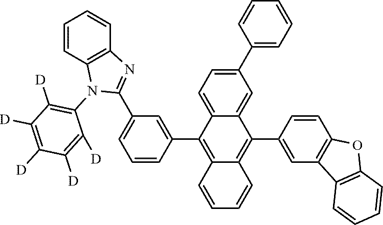 [SMILES: [2H]c1c([2H])c([2H])c(-n2c(-c3cccc(-c4c5ccccc5c(-c5ccc6oc7ccccc7c6c5)c5cc(-c6ccccc6)ccc45)c3)nc3ccccc32)c([2H])c1[2H]]

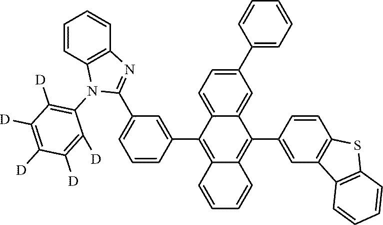 [SMILES: [2H]c1c([2H])c([2H])c(-n2c(-c3cccc(-c4c5ccccc5c(-c5ccc6sc7ccccc7c6c5)c5cc(-c6ccccc6)ccc45)c3)nc3ccccc32)c([2H])c1[2H]]

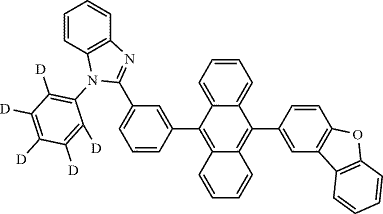 [SMILES: [2H]c1c([2H])c([2H])c(-n2c(-c3cccc(-c4c5ccccc5c(-c5ccc6oc7ccccc7c6c5)c5ccccc45)c3)nc3ccccc32)c([2H])c1[2H]]

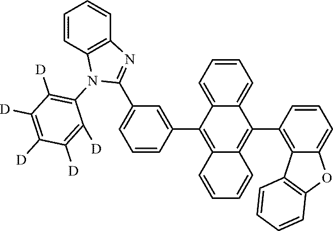 [SMILES: [2H]c1c([2H])c([2H])c(-n2c(-c3cccc(-c4c5ccccc5c(-c5cccc6oc7ccccc7c56)c5ccccc45)c3)nc3ccccc32)c([2H])c1[2H]]

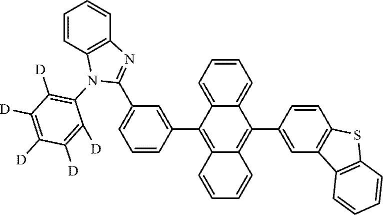 [SMILES: [2H]c1c([2H])c([2H])c(-n2c(-c3cccc(-c4c5ccccc5c(-c5ccc6sc7ccccc7c6c5)c5ccccc45)c3)nc3ccccc32)c([2H])c1[2H]]

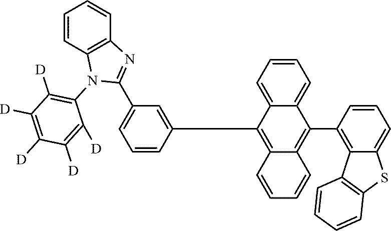 [SMILES: [2H]c1c([2H])c([2H])c(-n2c(-c3cccc(-c4c5ccccc5c(-c5cccc6sc7ccccc7c56)c5ccccc45)c3)nc3ccccc32)c([2H])c1[2H]]

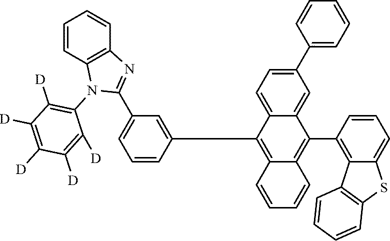 [SMILES: [2H]c1c([2H])c([2H])c(-n2c(-c3cccc(-c4c5ccccc5c(-c5cccc6sc7ccccc7c56)c5cc(-c6ccccc6)ccc45)c3)nc3ccccc32)c([2H])c1[2H]]

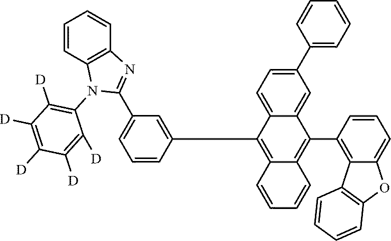 [SMILES: [2H]c1c([2H])c([2H])c(-n2c(-c3cccc(-c4c5ccccc5c(-c5cccc6oc7ccccc7c56)c5cc(-c6ccccc6)ccc45)c3)nc3ccccc32)c([2H])c1[2H]]

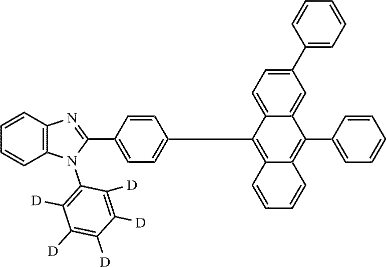 [SMILES: [2H]c1c([2H])c([2H])c(-n2c(-c3ccc(-c4c5ccccc5c(-c5ccccc5)c5cc(-c6ccccc6)ccc45)cc3)nc3ccccc32)c([2H])c1[2H]]

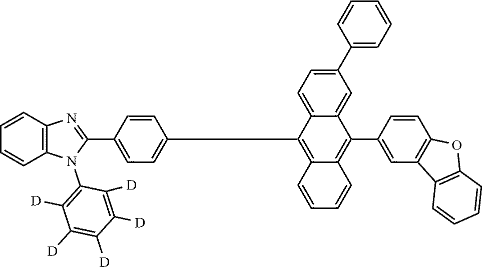 [SMILES: [2H]c1c([2H])c([2H])c(-n2c(-c3ccc(-c4c5ccccc5c(-c5ccc6oc7ccccc7c6c5)c5cc(-c6ccccc6)ccc45)cc3)nc3ccccc32)c([2H])c1[2H]]

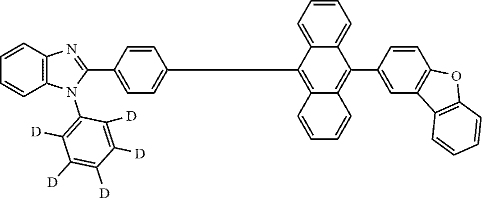 [SMILES: [2H]c1c([2H])c([2H])c(-n2c(-c3ccc(-c4c5ccccc5c(-c5ccc6oc7ccccc7c6c5)c5ccccc45)cc3)nc3ccccc32)c([2H])c1[2H]]

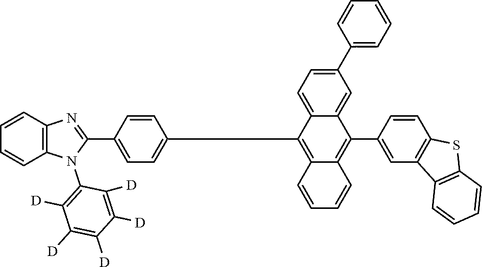 [SMILES: [2H]c1c([2H])c([2H])c(-n2c(-c3ccc(-c4c5ccccc5c(-c5ccc6sc7ccccc7c6c5)c5cc(-c6ccccc6)ccc45)cc3)nc3ccccc32)c([2H])c1[2H]]

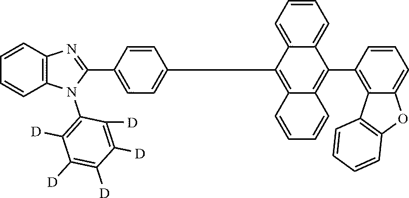 [SMILES: [2H]c1c([2H])c([2H])c(-n2c(-c3ccc(-c4c5ccccc5c(-c5cccc6oc7ccccc7c56)c5ccccc45)cc3)nc3ccccc32)c([2H])c1[2H]]

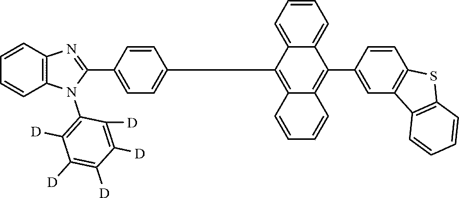 [SMILES: [2H]c1c([2H])c([2H])c(-n2c(-c3ccc(-c4c5ccccc5c(-c5ccc6sc7ccccc7c6c5)c5ccccc45)cc3)nc3ccccc32)c([2H])c1[2H]]

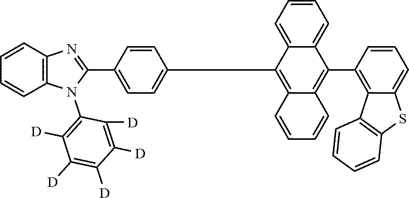 [SMILES: [2H]c1c([2H])c([2H])c(-n2c(-c3ccc(-c4c5ccccc5c(-c5cccc6sc7ccccc7c56)c5ccccc45)cc3)nc3ccccc32)c([2H])c1[2H]]

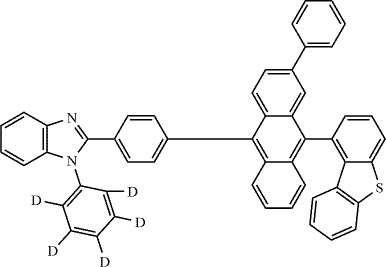 [SMILES: [2H]c1c([2H])c([2H])c(-n2c(-c3ccc(-c4c5ccccc5c(-c5cccc6sc7ccccc7c56)c5cc(-c6ccccc6)ccc45)cc3)nc3ccccc32)c([2H])c1[2H]]

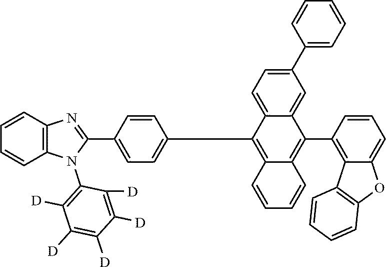 [SMILES: [2H]c1c([2H])c([2H])c(-n2c(-c3ccc(-c4c5ccccc5c(-c5cccc6oc7ccccc7c56)c5cc(-c6ccccc6)ccc45)cc3)nc3ccccc32)c([2H])c1[2H]]

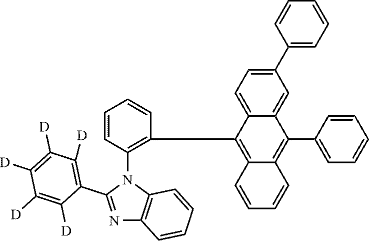 [SMILES: [2H]c1c([2H])c([2H])c(-c2nc3ccccc3n2-c2ccccc2-c2c3ccccc3c(-c3ccccc3)c3cc(-c4ccccc4)ccc23)c([2H])c1[2H]]

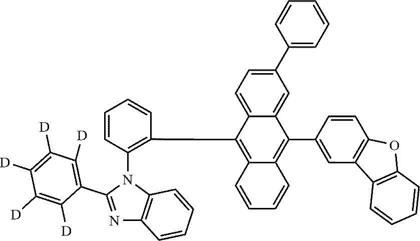 [SMILES: [2H]c1c([2H])c([2H])c(-c2nc3ccccc3n2-c2ccccc2-c2c3ccccc3c(-c3ccc4oc5ccccc5c4c3)c3cc(-c4ccccc4)ccc23)c([2H])c1[2H]]

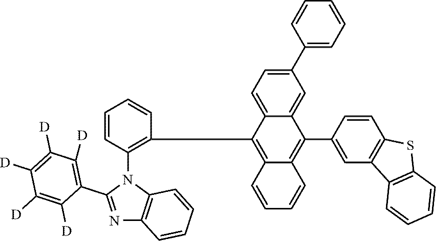 [SMILES: [2H]c1c([2H])c([2H])c(-c2nc3ccccc3n2-c2ccccc2-c2c3ccccc3c(-c3ccc4sc5ccccc5c4c3)c3cc(-c4ccccc4)ccc23)c([2H])c1[2H]]

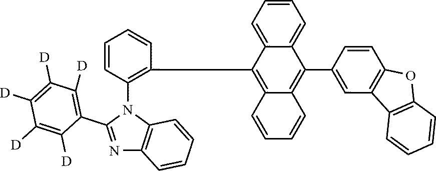 [SMILES: [2H]c1c([2H])c([2H])c(-c2nc3ccccc3n2-c2ccccc2-c2c3ccccc3c(-c3ccc4oc5ccccc5c4c3)c3ccccc23)c([2H])c1[2H]]

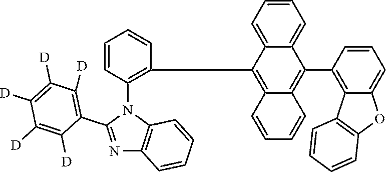 [SMILES: [2H]c1c([2H])c([2H])c(-c2nc3ccccc3n2-c2ccccc2-c2c3ccccc3c(-c3cccc4oc5ccccc5c34)c3ccccc23)c([2H])c1[2H]]

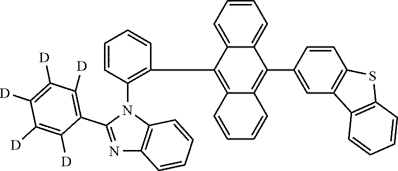 [SMILES: [2H]c1c([2H])c([2H])c(-c2nc3ccccc3n2-c2ccccc2-c2c3ccccc3c(-c3ccc4sc5ccccc5c4c3)c3ccccc23)c([2H])c1[2H]]

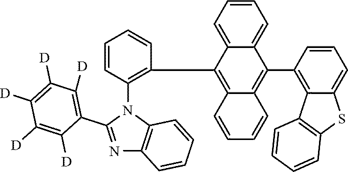 [SMILES: [2H]c1c([2H])c([2H])c(-c2nc3ccccc3n2-c2ccccc2-c2c3ccccc3c(-c3cccc4sc5ccccc5c34)c3ccccc23)c([2H])c1[2H]]

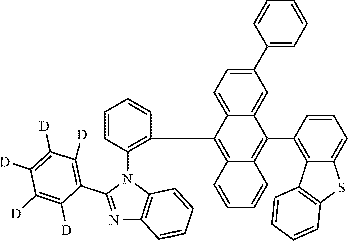 [SMILES: [2H]c1c([2H])c([2H])c(-c2nc3ccccc3n2-c2ccccc2-c2c3ccccc3c(-c3cccc4sc5ccccc5c34)c3cc(-c4ccccc4)ccc23)c([2H])c1[2H]]

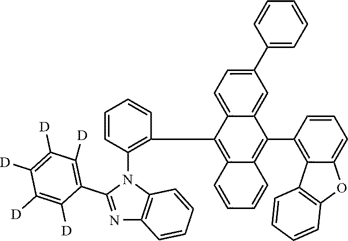 [SMILES: [2H]c1c([2H])c([2H])c(-c2nc3ccccc3n2-c2ccccc2-c2c3ccccc3c(-c3cccc4oc5ccccc5c34)c3cc(-c4ccccc4)ccc23)c([2H])c1[2H]]

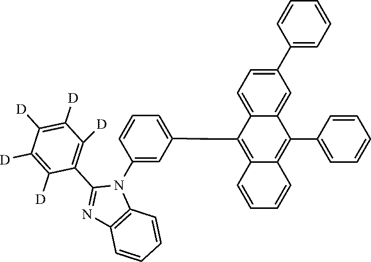 [SMILES: [2H]c1c([2H])c([2H])c(-c2nc3ccccc3n2-c2cccc(-c3c4ccccc4c(-c4ccccc4)c4cc(-c5ccccc5)ccc34)c2)c([2H])c1[2H]]

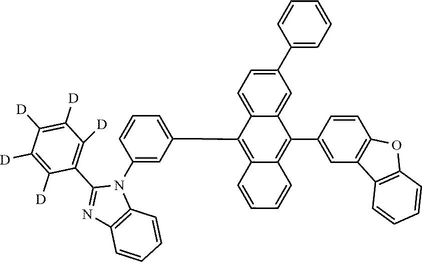 [SMILES: [2H]c1c([2H])c([2H])c(-c2nc3ccccc3n2-c2cccc(-c3c4ccccc4c(-c4ccc5oc6ccccc6c5c4)c4cc(-c5ccccc5)ccc34)c2)c([2H])c1[2H]]

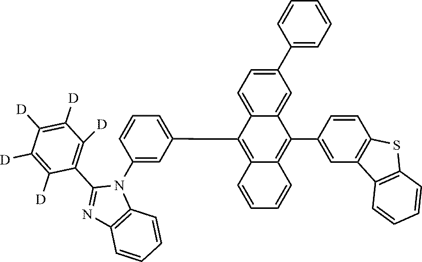 [SMILES: [2H]c1c([2H])c([2H])c(-c2nc3ccccc3n2-c2cccc(-c3c4ccccc4c(-c4ccc5sc6ccccc6c5c4)c4cc(-c5ccccc5)ccc34)c2)c([2H])c1[2H]]

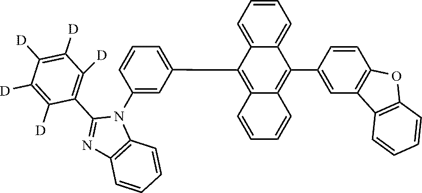 [SMILES: [2H]c1c([2H])c([2H])c(-c2nc3ccccc3n2-c2cccc(-c3c4ccccc4c(-c4ccc5oc6ccccc6c5c4)c4ccccc34)c2)c([2H])c1[2H]]

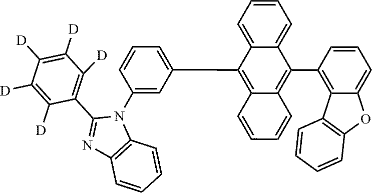 [SMILES: [2H]c1c([2H])c([2H])c(-c2nc3ccccc3n2-c2cccc(-c3c4ccccc4c(-c4cccc5oc6ccccc6c45)c4ccccc34)c2)c([2H])c1[2H]]

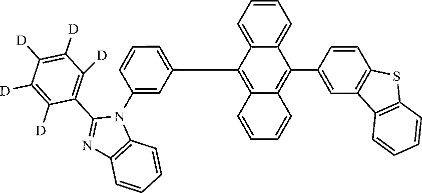 [SMILES: [2H]c1c([2H])c([2H])c(-c2nc3ccccc3n2-c2cccc(-c3c4ccccc4c(-c4ccc5sc6ccccc6c5c4)c4ccccc34)c2)c([2H])c1[2H]]

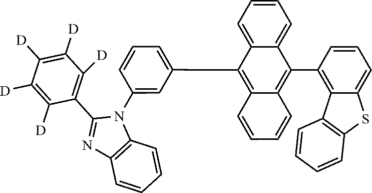 [SMILES: [2H]c1c([2H])c([2H])c(-c2nc3ccccc3n2-c2cccc(-c3c4ccccc4c(-c4cccc5sc6ccccc6c45)c4ccccc34)c2)c([2H])c1[2H]]

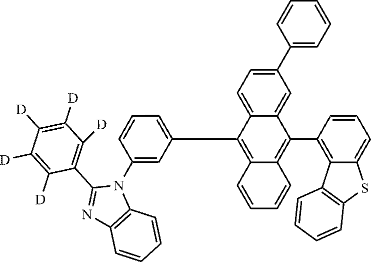 [SMILES: [2H]c1c([2H])c([2H])c(-c2nc3ccccc3n2-c2cccc(-c3c4ccccc4c(-c4cccc5sc6ccccc6c45)c4cc(-c5ccccc5)ccc34)c2)c([2H])c1[2H]]

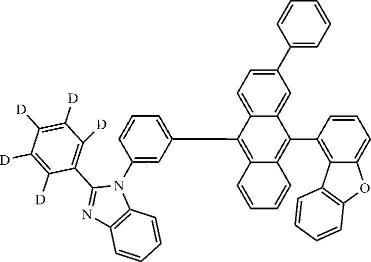 [SMILES: [2H]c1c([2H])c([2H])c(-c2nc3ccccc3n2-c2cccc(-c3c4ccccc4c(-c4cccc5oc6ccccc6c45)c4cc(-c5ccccc5)ccc34)c2)c([2H])c1[2H]]

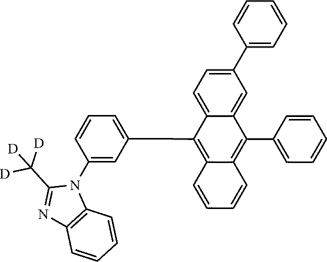 [SMILES: [2H]C([2H])([2H])c1nc2ccccc2n1-c1cccc(-c2c3ccccc3c(-c3ccccc3)c3cc(-c4ccccc4)ccc23)c1]

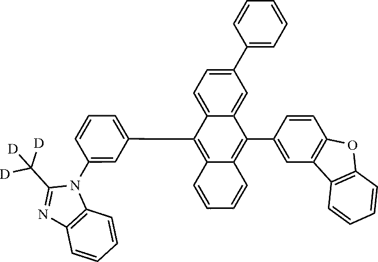 [SMILES: [2H]C([2H])([2H])c1nc2ccccc2n1-c1cccc(-c2c3ccccc3c(-c3ccc4oc5ccccc5c4c3)c3cc(-c4ccccc4)ccc23)c1]

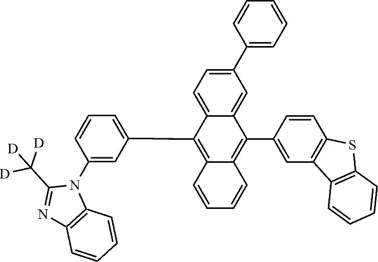 [SMILES: [2H]C([2H])([2H])c1nc2ccccc2n1-c1cccc(-c2c3ccccc3c(-c3ccc4sc5ccccc5c4c3)c3cc(-c4ccccc4)ccc23)c1]

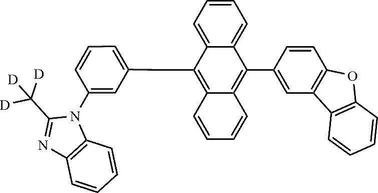 [SMILES: [2H]C([2H])([2H])c1nc2ccccc2n1-c1cccc(-c2c3ccccc3c(-c3ccc4oc5ccccc5c4c3)c3ccccc23)c1]

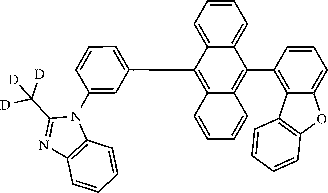 [SMILES: [2H]C([2H])([2H])c1nc2ccccc2n1-c1cccc(-c2c3ccccc3c(-c3cccc4oc5ccccc5c34)c3ccccc23)c1]

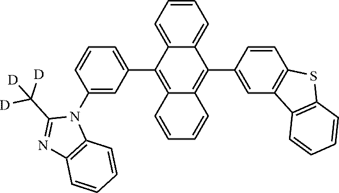 [SMILES: [2H]C([2H])([2H])c1nc2ccccc2n1-c1cccc(-c2c3ccccc3c(-c3ccc4sc5ccccc5c4c3)c3ccccc23)c1]

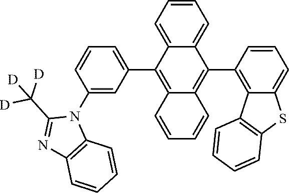 [SMILES: [2H]C([2H])([2H])c1nc2ccccc2n1-c1cccc(-c2c3ccccc3c(-c3cccc4sc5ccccc5c34)c3ccccc23)c1]

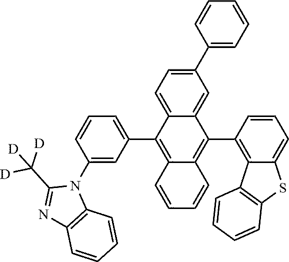 [SMILES: [2H]C([2H])([2H])c1nc2ccccc2n1-c1cccc(-c2c3ccccc3c(-c3cccc4sc5ccccc5c34)c3cc(-c4ccccc4)ccc23)c1]

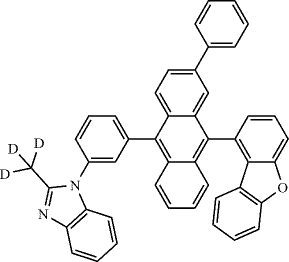 [SMILES: [2H]C([2H])([2H])c1nc2ccccc2n1-c1cccc(-c2c3ccccc3c(-c3cccc4oc5ccccc5c34)c3cc(-c4ccccc4)ccc23)c1]

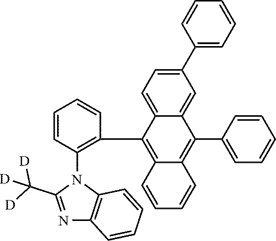 [SMILES: [2H]C([2H])([2H])c1nc2ccccc2n1-c1ccccc1-c1c2ccccc2c(-c2ccccc2)c2cc(-c3ccccc3)ccc12]

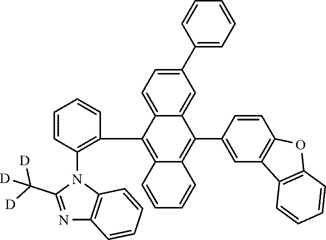 [SMILES: [2H]C([2H])([2H])c1nc2ccccc2n1-c1ccccc1-c1c2ccccc2c(-c2ccc3oc4ccccc4c3c2)c2cc(-c3ccccc3)ccc12]

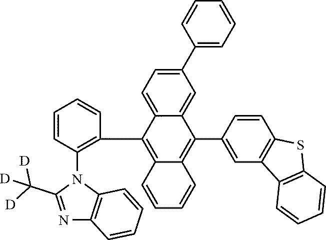 [SMILES: [2H]C([2H])([2H])c1nc2ccccc2n1-c1ccccc1-c1c2ccccc2c(-c2ccc3sc4ccccc4c3c2)c2cc(-c3ccccc3)ccc12]

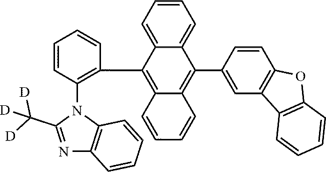 [SMILES: [2H]C([2H])([2H])c1nc2ccccc2n1-c1ccccc1-c1c2ccccc2c(-c2ccc3oc4ccccc4c3c2)c2ccccc12]

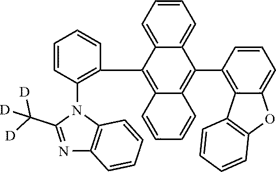 [SMILES: [2H]C([2H])([2H])c1nc2ccccc2n1-c1ccccc1-c1c2ccccc2c(-c2cccc3oc4ccccc4c23)c2ccccc12]

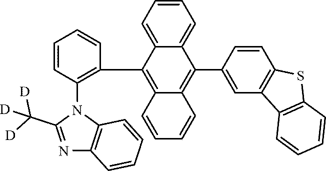 [SMILES: [2H]C([2H])([2H])c1nc2ccccc2n1-c1ccccc1-c1c2ccccc2c(-c2ccc3sc4ccccc4c3c2)c2ccccc12]

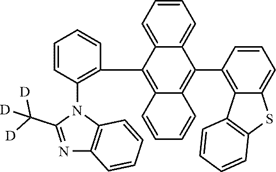 [SMILES: [2H]C([2H])([2H])c1nc2ccccc2n1-c1ccccc1-c1c2ccccc2c(-c2cccc3sc4ccccc4c23)c2ccccc12]

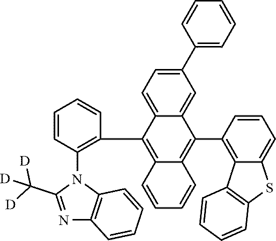 [SMILES: [2H]C([2H])([2H])c1nc2ccccc2n1-c1ccccc1-c1c2ccccc2c(-c2cccc3sc4ccccc4c23)c2cc(-c3ccccc3)ccc12]

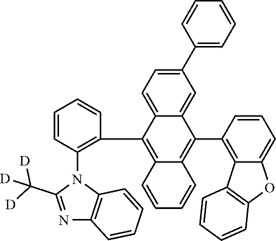 [SMILES: [2H]C([2H])([2H])c1nc2ccccc2n1-c1ccccc1-c1c2ccccc2c(-c2cccc3oc4ccccc4c23)c2cc(-c3ccccc3)ccc12]

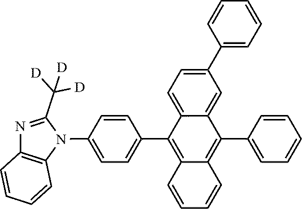 [SMILES: [2H]C([2H])([2H])c1nc2ccccc2n1-c1ccc(-c2c3ccccc3c(-c3ccccc3)c3cc(-c4ccccc4)ccc23)cc1]

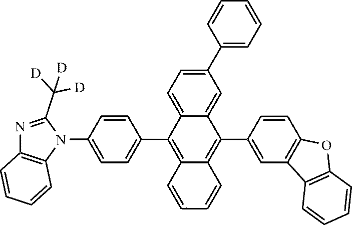 [SMILES: [2H]C([2H])([2H])c1nc2ccccc2n1-c1ccc(-c2c3ccccc3c(-c3ccc4oc5ccccc5c4c3)c3cc(-c4ccccc4)ccc23)cc1]

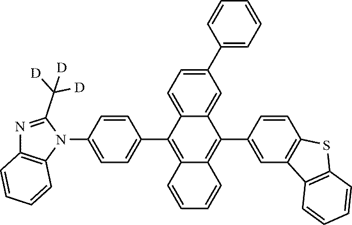 [SMILES: [2H]C([2H])([2H])c1nc2ccccc2n1-c1ccc(-c2c3ccccc3c(-c3ccc4sc5ccccc5c4c3)c3cc(-c4ccccc4)ccc23)cc1]

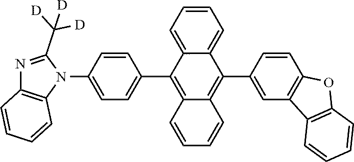 [SMILES: [2H]C([2H])([2H])c1nc2ccccc2n1-c1ccc(-c2c3ccccc3c(-c3ccc4oc5ccccc5c4c3)c3ccccc23)cc1]

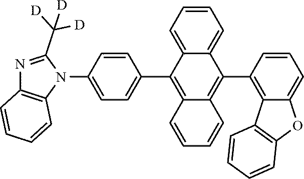 [SMILES: [2H]C([2H])([2H])c1nc2ccccc2n1-c1ccc(-c2c3ccccc3c(-c3cccc4oc5ccccc5c34)c3ccccc23)cc1]

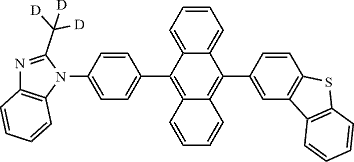 [SMILES: [2H]C([2H])([2H])c1nc2ccccc2n1-c1ccc(-c2c3ccccc3c(-c3ccc4sc5ccccc5c4c3)c3ccccc23)cc1]

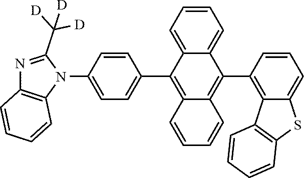 [SMILES: [2H]C([2H])([2H])c1nc2ccccc2n1-c1ccc(-c2c3ccccc3c(-c3cccc4sc5ccccc5c34)c3ccccc23)cc1]

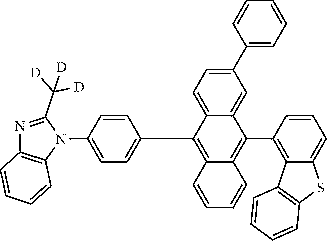 [SMILES: [2H]C([2H])([2H])c1nc2ccccc2n1-c1ccc(-c2c3ccccc3c(-c3cccc4sc5ccccc5c34)c3cc(-c4ccccc4)ccc23)cc1]

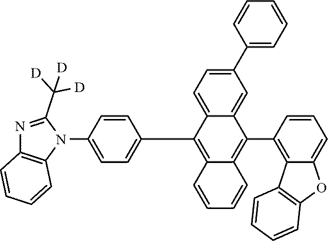 [SMILES: [2H]C([2H])([2H])c1nc2ccccc2n1-c1ccc(-c2c3ccccc3c(-c3cccc4oc5ccccc5c34)c3cc(-c4ccccc4)ccc23)cc1]

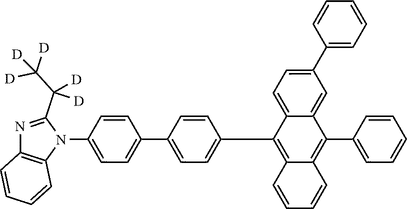 [SMILES: [2H]C([2H])([2H])C([2H])([2H])c1nc2ccccc2n1-c1ccc(-c2ccc(-c3c4ccccc4c(-c4ccccc4)c4cc(-c5ccccc5)ccc34)cc2)cc1]

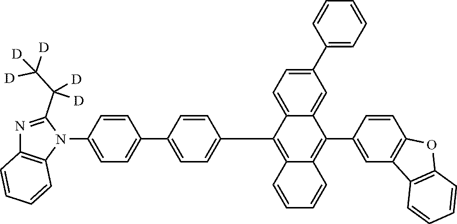 [SMILES: [2H]C([2H])([2H])C([2H])([2H])c1nc2ccccc2n1-c1ccc(-c2ccc(-c3c4ccccc4c(-c4ccc5oc6ccccc6c5c4)c4cc(-c5ccccc5)ccc34)cc2)cc1]

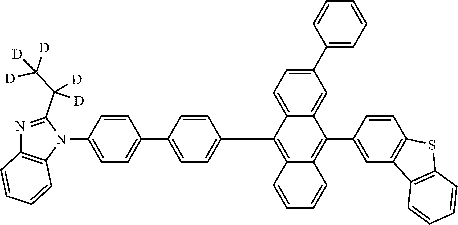 [SMILES: [2H]C([2H])([2H])C([2H])([2H])c1nc2ccccc2n1-c1ccc(-c2ccc(-c3c4ccccc4c(-c4ccc5sc6ccccc6c5c4)c4cc(-c5ccccc5)ccc34)cc2)cc1]

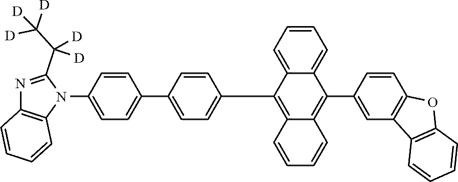 [SMILES: [2H]C([2H])([2H])C([2H])([2H])c1nc2ccccc2n1-c1ccc(-c2ccc(-c3c4ccccc4c(-c4ccc5oc6ccccc6c5c4)c4ccccc34)cc2)cc1]

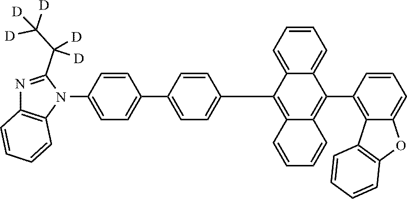 [SMILES: [2H]C([2H])([2H])C([2H])([2H])c1nc2ccccc2n1-c1ccc(-c2ccc(-c3c4ccccc4c(-c4cccc5oc6ccccc6c45)c4ccccc34)cc2)cc1]

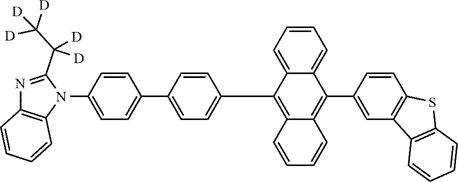 [SMILES: [2H]C([2H])([2H])C([2H])([2H])c1nc2ccccc2n1-c1ccc(-c2ccc(-c3c4ccccc4c(-c4ccc5sc6ccccc6c5c4)c4ccccc34)cc2)cc1]

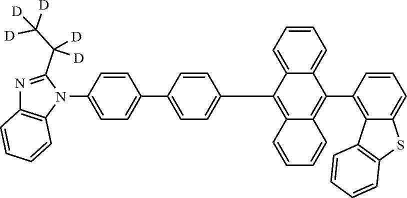 [SMILES: [2H]C([2H])([2H])C([2H])([2H])c1nc2ccccc2n1-c1ccc(-c2ccc(-c3c4ccccc4c(-c4cccc5sc6ccccc6c45)c4ccccc34)cc2)cc1]

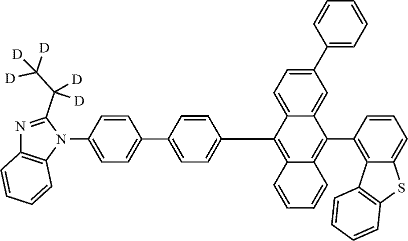 [SMILES: [2H]C([2H])([2H])C([2H])([2H])c1nc2ccccc2n1-c1ccc(-c2ccc(-c3c4ccccc4c(-c4cccc5sc6ccccc6c45)c4cc(-c5ccccc5)ccc34)cc2)cc1]

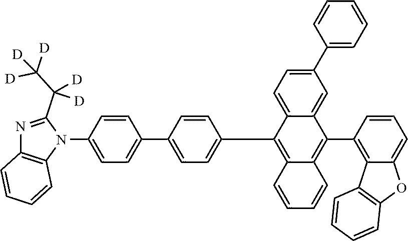 [SMILES: [2H]C([2H])([2H])C([2H])([2H])c1nc2ccccc2n1-c1ccc(-c2ccc(-c3c4ccccc4c(-c4cccc5oc6ccccc6c45)c4cc(-c5ccccc5)ccc34)cc2)cc1]

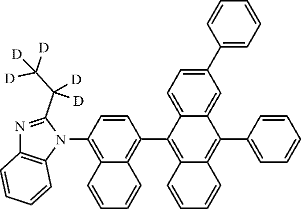 [SMILES: [2H]C([2H])([2H])C([2H])([2H])c1nc2ccccc2n1-c1ccc(-c2c3ccccc3c(-c3ccccc3)c3cc(-c4ccccc4)ccc23)c2ccccc12]

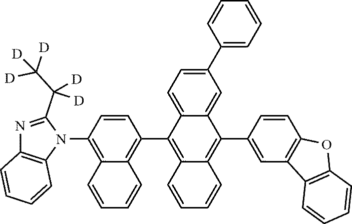 [SMILES: [2H]C([2H])([2H])C([2H])([2H])c1nc2ccccc2n1-c1ccc(-c2c3ccccc3c(-c3ccc4oc5ccccc5c4c3)c3cc(-c4ccccc4)ccc23)c2ccccc12]

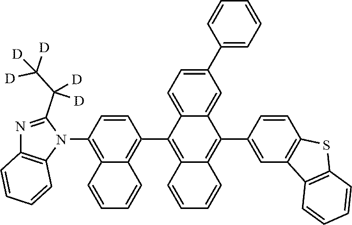 [SMILES: [2H]C([2H])([2H])C([2H])([2H])c1nc2ccccc2n1-c1ccc(-c2c3ccccc3c(-c3ccc4sc5ccccc5c4c3)c3cc(-c4ccccc4)ccc23)c2ccccc12]

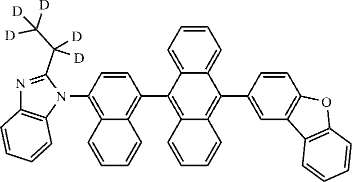 [SMILES: [2H]C([2H])([2H])C([2H])([2H])c1nc2ccccc2n1-c1ccc(-c2c3ccccc3c(-c3ccc4oc5ccccc5c4c3)c3ccccc23)c2ccccc12]

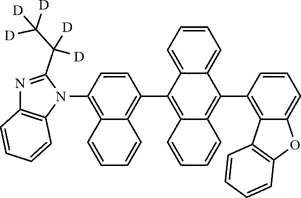 [SMILES: [2H]C([2H])([2H])C([2H])([2H])c1nc2ccccc2n1-c1ccc(-c2c3ccccc3c(-c3cccc4oc5ccccc5c34)c3ccccc23)c2ccccc12]

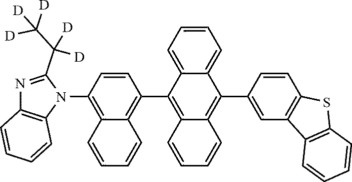 [SMILES: [2H]C([2H])([2H])C([2H])([2H])c1nc2ccccc2n1-c1ccc(-c2c3ccccc3c(-c3ccc4sc5ccccc5c4c3)c3ccccc23)c2ccccc12]

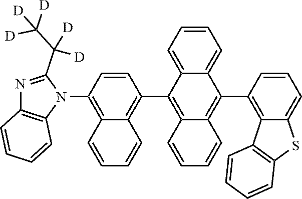 [SMILES: [2H]C([2H])([2H])C([2H])([2H])c1nc2ccccc2n1-c1ccc(-c2c3ccccc3c(-c3cccc4sc5ccccc5c34)c3ccccc23)c2ccccc12]

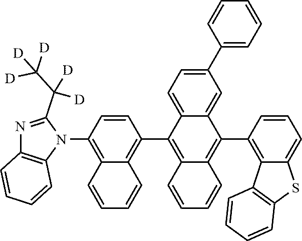 [SMILES: [2H]C([2H])([2H])C([2H])([2H])c1nc2ccccc2n1-c1ccc(-c2c3ccccc3c(-c3cccc4sc5ccccc5c34)c3cc(-c4ccccc4)ccc23)c2ccccc12]

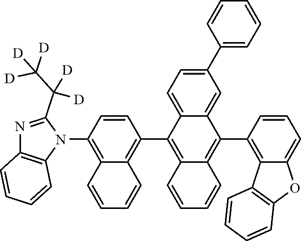 [SMILES: [2H]C([2H])([2H])C([2H])([2H])c1nc2ccccc2n1-c1ccc(-c2c3ccccc3c(-c3cccc4oc5ccccc5c34)c3cc(-c4ccccc4)ccc23)c2ccccc12]

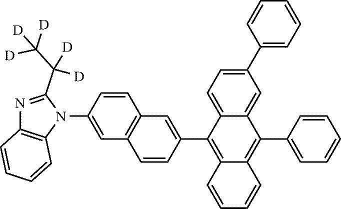 [SMILES: [2H]C([2H])([2H])C([2H])([2H])c1nc2ccccc2n1-c1ccc2cc(-c3c4ccccc4c(-c4ccccc4)c4cc(-c5ccccc5)ccc34)ccc2c1]

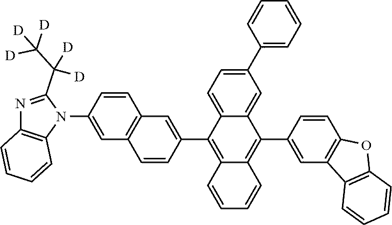 [SMILES: [2H]C([2H])([2H])C([2H])([2H])c1nc2ccccc2n1-c1ccc2cc(-c3c4ccccc4c(-c4ccc5oc6ccccc6c5c4)c4cc(-c5ccccc5)ccc34)ccc2c1]

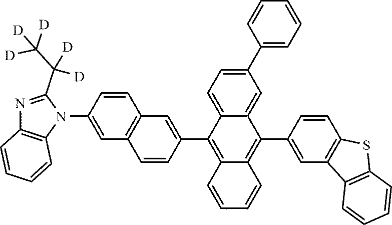 [SMILES: [2H]C([2H])([2H])C([2H])([2H])c1nc2ccccc2n1-c1ccc2cc(-c3c4ccccc4c(-c4ccc5sc6ccccc6c5c4)c4cc(-c5ccccc5)ccc34)ccc2c1]

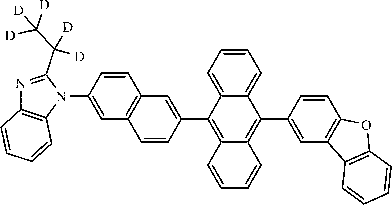 [SMILES: [2H]C([2H])([2H])C([2H])([2H])c1nc2ccccc2n1-c1ccc2cc(-c3c4ccccc4c(-c4ccc5oc6ccccc6c5c4)c4ccccc34)ccc2c1]

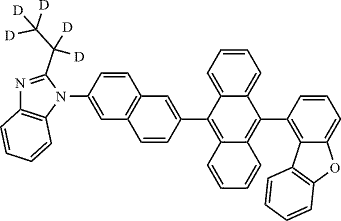 [SMILES: [2H]C([2H])([2H])C([2H])([2H])c1nc2ccccc2n1-c1ccc2cc(-c3c4ccccc4c(-c4cccc5oc6ccccc6c45)c4ccccc34)ccc2c1]

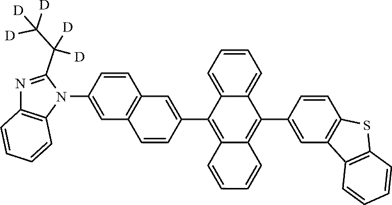 [SMILES: [2H]C([2H])([2H])C([2H])([2H])c1nc2ccccc2n1-c1ccc2cc(-c3c4ccccc4c(-c4ccc5sc6ccccc6c5c4)c4ccccc34)ccc2c1]

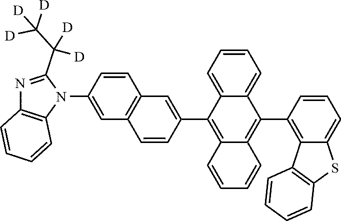 [SMILES: [2H]C([2H])([2H])C([2H])([2H])c1nc2ccccc2n1-c1ccc2cc(-c3c4ccccc4c(-c4cccc5sc6ccccc6c45)c4ccccc34)ccc2c1]

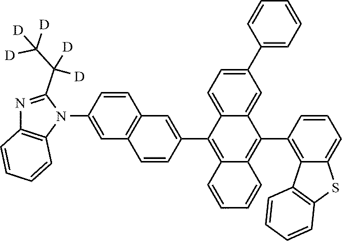 [SMILES: [2H]C([2H])([2H])C([2H])([2H])c1nc2ccccc2n1-c1ccc2cc(-c3c4ccccc4c(-c4cccc5sc6ccccc6c45)c4cc(-c5ccccc5)ccc34)ccc2c1]

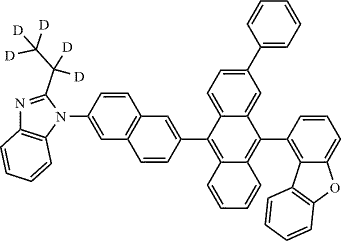 [SMILES: [2H]C([2H])([2H])C([2H])([2H])c1nc2ccccc2n1-c1ccc2cc(-c3c4ccccc4c(-c4cccc5oc6ccccc6c45)c4cc(-c5ccccc5)ccc34)ccc2c1]

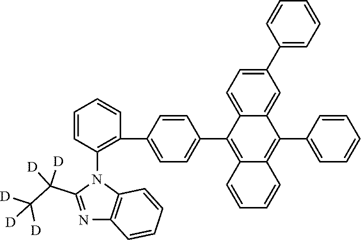 [SMILES: [2H]C([2H])([2H])C([2H])([2H])c1nc2ccccc2n1-c1ccccc1-c1ccc(-c2c3ccccc3c(-c3ccccc3)c3cc(-c4ccccc4)ccc23)cc1]

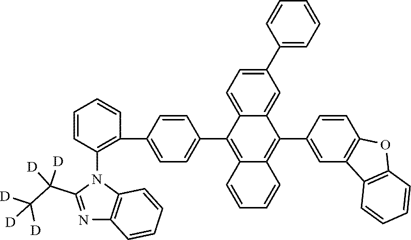 [SMILES: [2H]C([2H])([2H])C([2H])([2H])c1nc2ccccc2n1-c1ccccc1-c1ccc(-c2c3ccccc3c(-c3ccc4oc5ccccc5c4c3)c3cc(-c4ccccc4)ccc23)cc1]

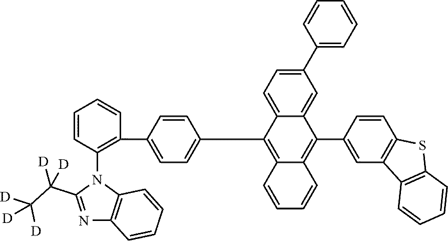 [SMILES: [2H]C([2H])([2H])C([2H])([2H])c1nc2ccccc2n1-c1ccccc1-c1ccc(-c2c3ccccc3c(-c3ccc4sc5ccccc5c4c3)c3cc(-c4ccccc4)ccc23)cc1]

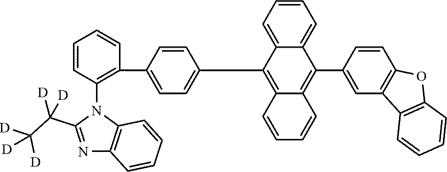 [SMILES: [2H]C([2H])([2H])C([2H])([2H])c1nc2ccccc2n1-c1ccccc1-c1ccc(-c2c3ccccc3c(-c3ccc4oc5ccccc5c4c3)c3ccccc23)cc1]

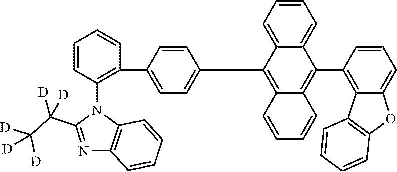 [SMILES: [2H]C([2H])([2H])C([2H])([2H])c1nc2ccccc2n1-c1ccccc1-c1ccc(-c2c3ccccc3c(-c3cccc4oc5ccccc5c34)c3ccccc23)cc1]

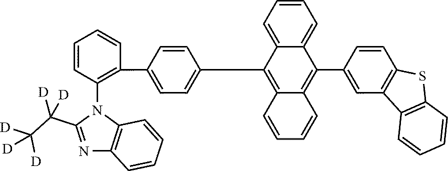 [SMILES: [2H]C([2H])([2H])C([2H])([2H])c1nc2ccccc2n1-c1ccccc1-c1ccc(-c2c3ccccc3c(-c3ccc4sc5ccccc5c4c3)c3ccccc23)cc1]

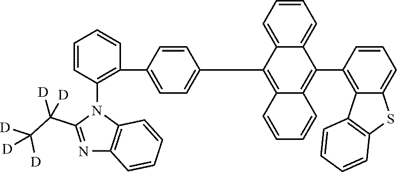 [SMILES: [2H]C([2H])([2H])C([2H])([2H])c1nc2ccccc2n1-c1ccccc1-c1ccc(-c2c3ccccc3c(-c3cccc4sc5ccccc5c34)c3ccccc23)cc1]

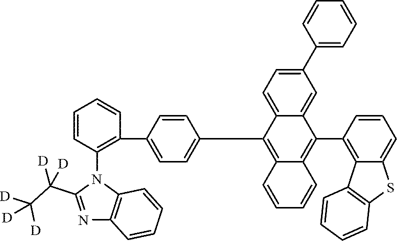 [SMILES: [2H]C([2H])([2H])C([2H])([2H])c1nc2ccccc2n1-c1ccccc1-c1ccc(-c2c3ccccc3c(-c3cccc4sc5ccccc5c34)c3cc(-c4ccccc4)ccc23)cc1]

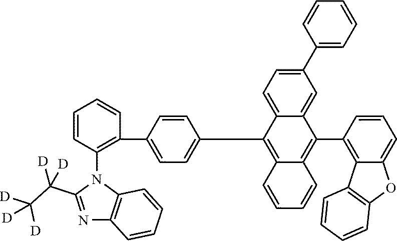 [SMILES: [2H]C([2H])([2H])C([2H])([2H])c1nc2ccccc2n1-c1ccccc1-c1ccc(-c2c3ccccc3c(-c3cccc4oc5ccccc5c34)c3cc(-c4ccccc4)ccc23)cc1]

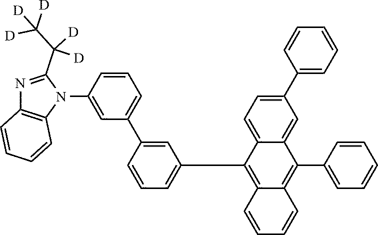 [SMILES: [2H]C([2H])([2H])C([2H])([2H])c1nc2ccccc2n1-c1cccc(-c2cccc(-c3c4ccccc4c(-c4ccccc4)c4cc(-c5ccccc5)ccc34)c2)c1]

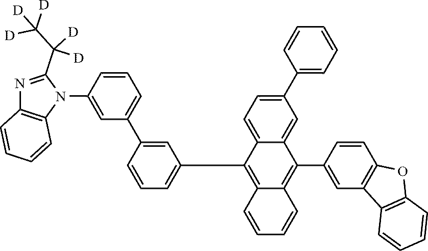 [SMILES: [2H]C([2H])([2H])C([2H])([2H])c1nc2ccccc2n1-c1cccc(-c2cccc(-c3c4ccccc4c(-c4ccc5oc6ccccc6c5c4)c4cc(-c5ccccc5)ccc34)c2)c1]

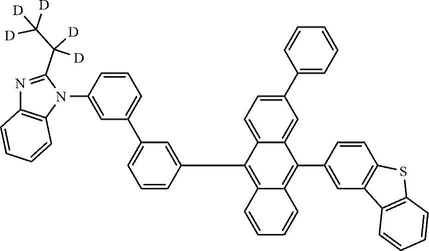 [SMILES: [2H]C([2H])([2H])C([2H])([2H])c1nc2ccccc2n1-c1cccc(-c2cccc(-c3c4ccccc4c(-c4ccc5sc6ccccc6c5c4)c4cc(-c5ccccc5)ccc34)c2)c1]

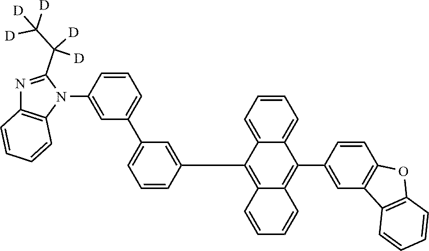 [SMILES: [2H]C([2H])([2H])C([2H])([2H])c1nc2ccccc2n1-c1cccc(-c2cccc(-c3c4ccccc4c(-c4ccc5oc6ccccc6c5c4)c4ccccc34)c2)c1]

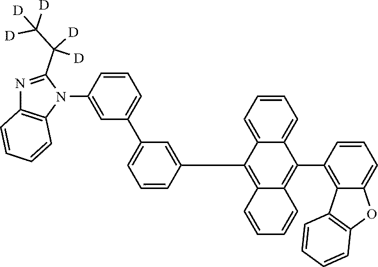 [SMILES: [2H]C([2H])([2H])C([2H])([2H])c1nc2ccccc2n1-c1cccc(-c2cccc(-c3c4ccccc4c(-c4cccc5oc6ccccc6c45)c4ccccc34)c2)c1]

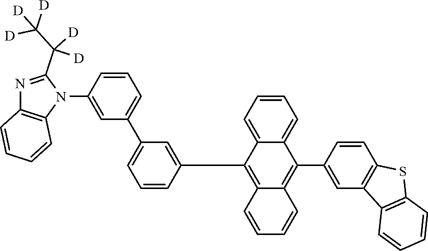 [SMILES: [2H]C([2H])([2H])C([2H])([2H])c1nc2ccccc2n1-c1cccc(-c2cccc(-c3c4ccccc4c(-c4ccc5sc6ccccc6c5c4)c4ccccc34)c2)c1]

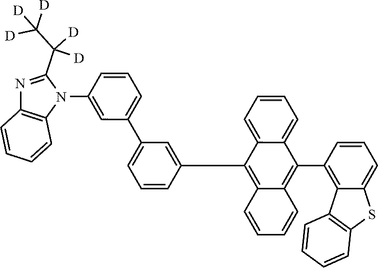 [SMILES: [2H]C([2H])([2H])C([2H])([2H])c1nc2ccccc2n1-c1cccc(-c2cccc(-c3c4ccccc4c(-c4cccc5sc6ccccc6c45)c4ccccc34)c2)c1]

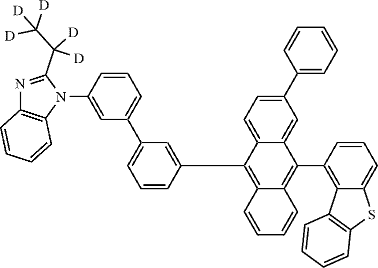 [SMILES: [2H]C([2H])([2H])C([2H])([2H])c1nc2ccccc2n1-c1cccc(-c2cccc(-c3c4ccccc4c(-c4cccc5sc6ccccc6c45)c4cc(-c5ccccc5)ccc34)c2)c1]

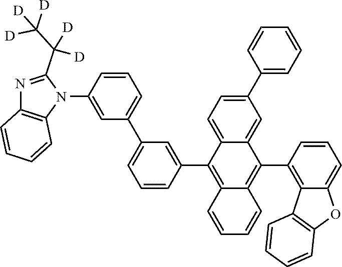 [SMILES: [2H]C([2H])([2H])C([2H])([2H])c1nc2ccccc2n1-c1cccc(-c2cccc(-c3c4ccccc4c(-c4cccc5oc6ccccc6c45)c4cc(-c5ccccc5)ccc34)c2)c1]

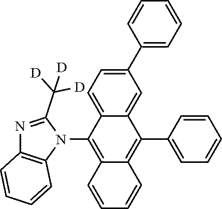 [SMILES: [2H]C([2H])([2H])c1nc2ccccc2n1-c1c2ccccc2c(-c2ccccc2)c2cc(-c3ccccc3)ccc12]

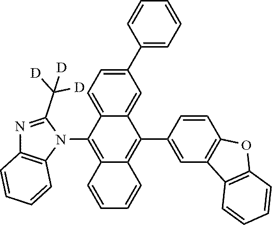 [SMILES: [2H]C([2H])([2H])c1nc2ccccc2n1-c1c2ccccc2c(-c2ccc3oc4ccccc4c3c2)c2cc(-c3ccccc3)ccc12]

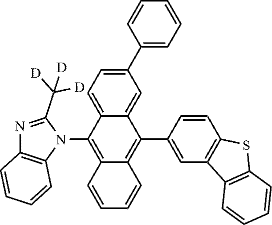 [SMILES: [2H]C([2H])([2H])c1nc2ccccc2n1-c1c2ccccc2c(-c2ccc3sc4ccccc4c3c2)c2cc(-c3ccccc3)ccc12]

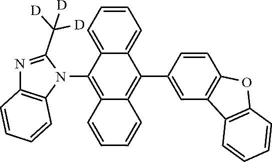 [SMILES: [2H]C([2H])([2H])c1nc2ccccc2n1-c1c2ccccc2c(-c2ccc3oc4ccccc4c3c2)c2ccccc12]

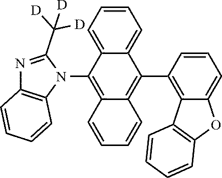 [SMILES: [2H]C([2H])([2H])c1nc2ccccc2n1-c1c2ccccc2c(-c2cccc3oc4ccccc4c23)c2ccccc12]

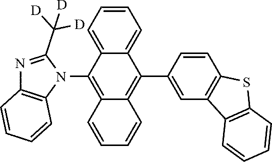 [SMILES: [2H]C([2H])([2H])c1nc2ccccc2n1-c1c2ccccc2c(-c2ccc3sc4ccccc4c3c2)c2ccccc12]

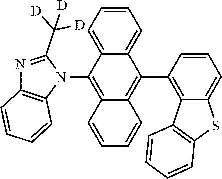 [SMILES: [2H]C([2H])([2H])c1nc2ccccc2n1-c1c2ccccc2c(-c2cccc3sc4ccccc4c23)c2ccccc12]

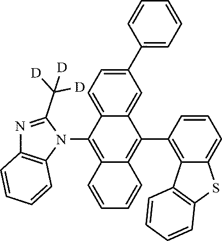 [SMILES: [2H]C([2H])([2H])c1nc2ccccc2n1-c1c2ccccc2c(-c2cccc3sc4ccccc4c23)c2cc(-c3ccccc3)ccc12]

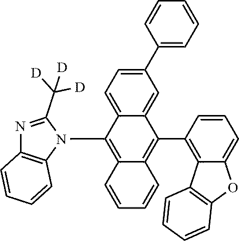 [SMILES: [2H]C([2H])([2H])c1nc2ccccc2n1-c1c2ccccc2c(-c2cccc3oc4ccccc4c23)c2cc(-c3ccccc3)ccc12]

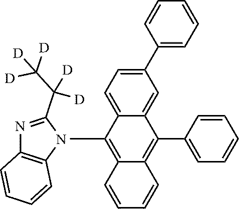 [SMILES: [2H]C([2H])([2H])C([2H])([2H])c1nc2ccccc2n1-c1c2ccccc2c(-c2ccccc2)c2cc(-c3ccccc3)ccc12]

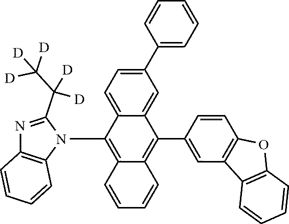 [SMILES: [2H]C([2H])([2H])C([2H])([2H])c1nc2ccccc2n1-c1c2ccccc2c(-c2ccc3oc4ccccc4c3c2)c2cc(-c3ccccc3)ccc12]

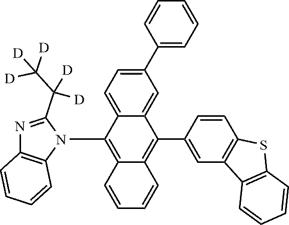 [SMILES: [2H]C([2H])([2H])C([2H])([2H])c1nc2ccccc2n1-c1c2ccccc2c(-c2ccc3sc4ccccc4c3c2)c2cc(-c3ccccc3)ccc12]

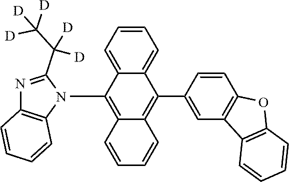 [SMILES: [2H]C([2H])([2H])C([2H])([2H])c1nc2ccccc2n1-c1c2ccccc2c(-c2ccc3oc4ccccc4c3c2)c2ccccc12]

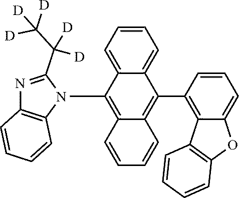 [SMILES: [2H]C([2H])([2H])C([2H])([2H])c1nc2ccccc2n1-c1c2ccccc2c(-c2cccc3oc4ccccc4c23)c2ccccc12]

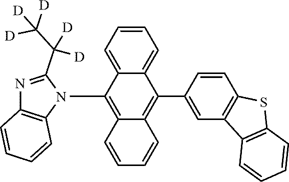 [SMILES: [2H]C([2H])([2H])C([2H])([2H])c1nc2ccccc2n1-c1c2ccccc2c(-c2ccc3sc4ccccc4c3c2)c2ccccc12]

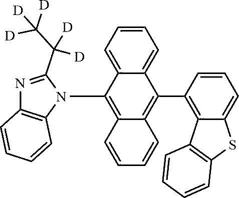 [SMILES: [2H]C([2H])([2H])C([2H])([2H])c1nc2ccccc2n1-c1c2ccccc2c(-c2cccc3sc4ccccc4c23)c2ccccc12]

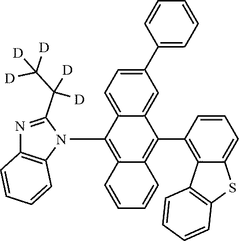 [SMILES: [2H]C([2H])([2H])C([2H])([2H])c1nc2ccccc2n1-c1c2ccccc2c(-c2cccc3sc4ccccc4c23)c2cc(-c3ccccc3)ccc12]

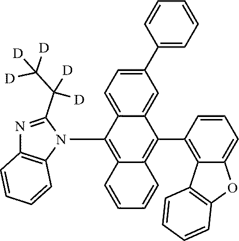 [SMILES: [2H]C([2H])([2H])C([2H])([2H])c1nc2ccccc2n1-c1c2ccccc2c(-c2cccc3oc4ccccc4c23)c2cc(-c3ccccc3)ccc12]

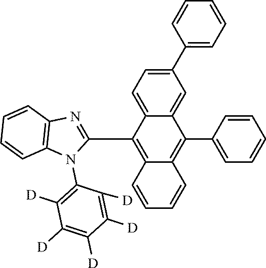 [SMILES: [2H]c1c([2H])c([2H])c(-n2c(-c3c4ccccc4c(-c4ccccc4)c4cc(-c5ccccc5)ccc34)nc3ccccc32)c([2H])c1[2H]]

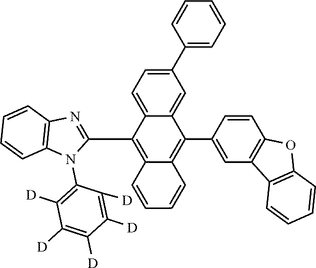 [SMILES: [2H]c1c([2H])c([2H])c(-n2c(-c3c4ccccc4c(-c4ccc5oc6ccccc6c5c4)c4cc(-c5ccccc5)ccc34)nc3ccccc32)c([2H])c1[2H]]

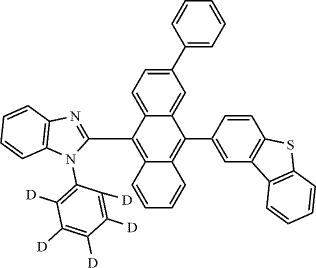 [SMILES: [2H]c1c([2H])c([2H])c(-n2c(-c3c4ccccc4c(-c4ccc5sc6ccccc6c5c4)c4cc(-c5ccccc5)ccc34)nc3ccccc32)c([2H])c1[2H]]

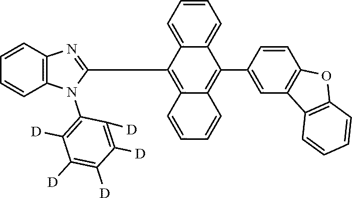 [SMILES: [2H]c1c([2H])c([2H])c(-n2c(-c3c4ccccc4c(-c4ccc5oc6ccccc6c5c4)c4ccccc34)nc3ccccc32)c([2H])c1[2H]]

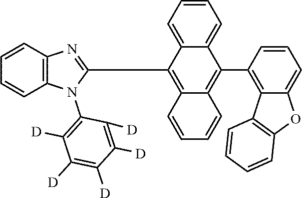 [SMILES: [2H]c1c([2H])c([2H])c(-n2c(-c3c4ccccc4c(-c4cccc5oc6ccccc6c45)c4ccccc34)nc3ccccc32)c([2H])c1[2H]]

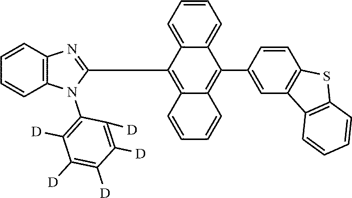 [SMILES: [2H]c1c([2H])c([2H])c(-n2c(-c3c4ccccc4c(-c4ccc5sc6ccccc6c5c4)c4ccccc34)nc3ccccc32)c([2H])c1[2H]]

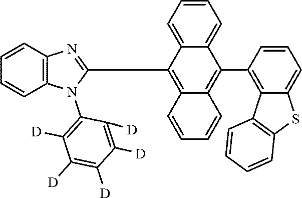 [SMILES: [2H]c1c([2H])c([2H])c(-n2c(-c3c4ccccc4c(-c4cccc5sc6ccccc6c45)c4ccccc34)nc3ccccc32)c([2H])c1[2H]]

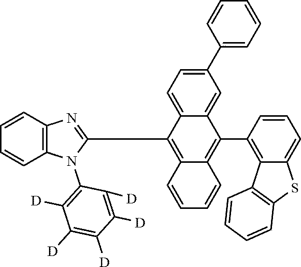 [SMILES: [2H]c1c([2H])c([2H])c(-n2c(-c3c4ccccc4c(-c4cccc5sc6ccccc6c45)c4cc(-c5ccccc5)ccc34)nc3ccccc32)c([2H])c1[2H]]

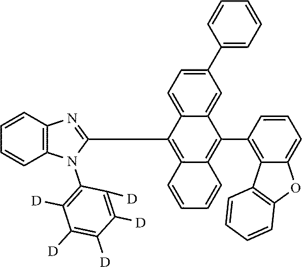 [SMILES: [2H]c1c([2H])c([2H])c(-n2c(-c3c4ccccc4c(-c4cccc5oc6ccccc6c45)c4cc(-c5ccccc5)ccc34)nc3ccccc32)c([2H])c1[2H]]

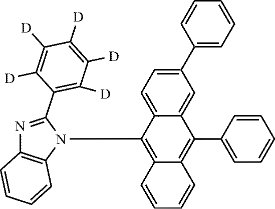 [SMILES: [2H]c1c([2H])c([2H])c(-c2nc3ccccc3n2-c2c3ccccc3c(-c3ccccc3)c3cc(-c4ccccc4)ccc23)c([2H])c1[2H]]

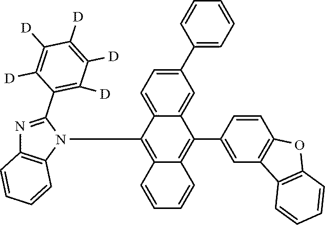 [SMILES: [2H]c1c([2H])c([2H])c(-c2nc3ccccc3n2-c2c3ccccc3c(-c3ccc4oc5ccccc5c4c3)c3cc(-c4ccccc4)ccc23)c([2H])c1[2H]]

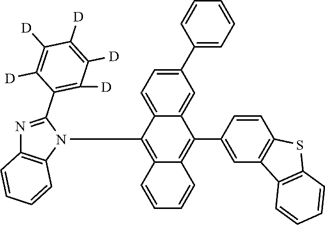 [SMILES: [2H]c1c([2H])c([2H])c(-c2nc3ccccc3n2-c2c3ccccc3c(-c3ccc4sc5ccccc5c4c3)c3cc(-c4ccccc4)ccc23)c([2H])c1[2H]]

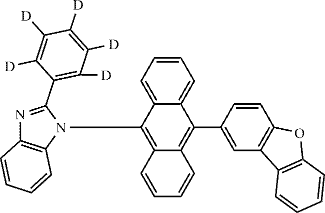 [SMILES: [2H]c1c([2H])c([2H])c(-c2nc3ccccc3n2-c2c3ccccc3c(-c3ccc4oc5ccccc5c4c3)c3ccccc23)c([2H])c1[2H]]

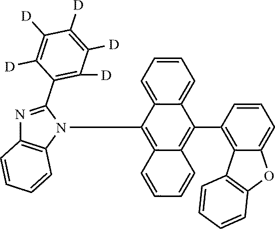 [SMILES: [2H]c1c([2H])c([2H])c(-c2nc3ccccc3n2-c2c3ccccc3c(-c3cccc4oc5ccccc5c34)c3ccccc23)c([2H])c1[2H]]

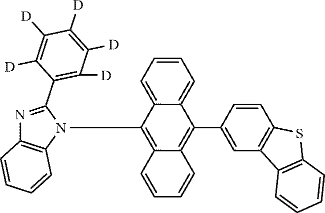 [SMILES: [2H]c1c([2H])c([2H])c(-c2nc3ccccc3n2-c2c3ccccc3c(-c3ccc4sc5ccccc5c4c3)c3ccccc23)c([2H])c1[2H]]

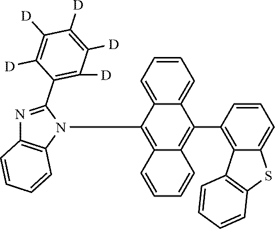 [SMILES: [2H]c1c([2H])c([2H])c(-c2nc3ccccc3n2-c2c3ccccc3c(-c3cccc4sc5ccccc5c34)c3ccccc23)c([2H])c1[2H]]

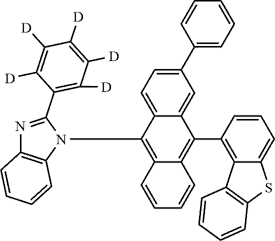 [SMILES: [2H]c1c([2H])c([2H])c(-c2nc3ccccc3n2-c2c3ccccc3c(-c3cccc4sc5ccccc5c34)c3cc(-c4ccccc4)ccc23)c([2H])c1[2H]]

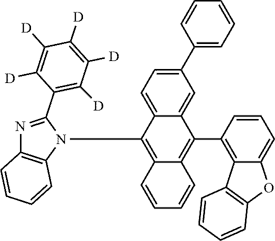 [SMILES: [2H]c1c([2H])c([2H])c(-c2nc3ccccc3n2-c2c3ccccc3c(-c3cccc4oc5ccccc5c34)c3cc(-c4ccccc4)ccc23)c([2H])c1[2H]]

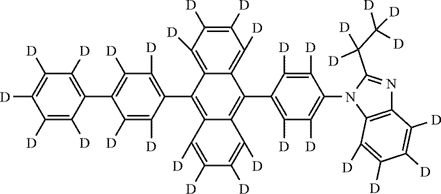 [SMILES: [2H]c1c([2H])c([2H])c(-c2c([2H])c([2H])c(-c3c4c([2H])c([2H])c([2H])c([2H])c4c(-c4c([2H])c([2H])c(-n5c(C([2H])([2H])C([2H])([2H])[2H])nc6c([2H])c([2H])c([2H])c([2H])c65)c([2H])c4[2H])c4c([2H])c([2H])c([2H])c([2H])c34)c([2H])c2[2H])c([2H])c1[2H]]

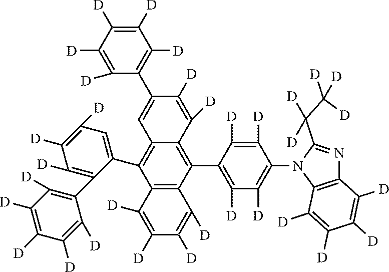 [SMILES: [2H]c1cc(-c2c3cc(-c4c([2H])c([2H])c([2H])c([2H])c4[2H])c([2H])c([2H])c3c(-c3c([2H])c([2H])c(-n4c(C([2H])([2H])C([2H])([2H])[2H])nc5c([2H])c([2H])c([2H])c([2H])c54)c([2H])c3[2H])c3c([2H])c([2H])c([2H])c([2H])c23)c(-c2c([2H])c([2H])c([2H])c([2H])c2[2H])c([2H])c1[2H]]

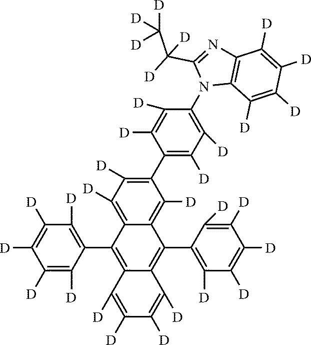 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c([2H])c(-n5c(C([2H])([2H])C([2H])([2H])[2H])nc6c([2H])c([2H])c([2H])c([2H])c65)c([2H])c4[2H])c([2H])c([2H])c23)c([2H])c1[2H]]

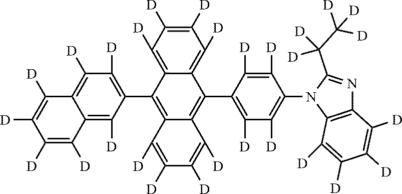 [SMILES: [2H]c1c([2H])c(-n2c(C([2H])([2H])C([2H])([2H])[2H])nc3c([2H])c([2H])c([2H])c([2H])c32)c([2H])c([2H])c1-c1c2c([2H])c([2H])c([2H])c([2H])c2c(-c2c([2H])c([2H])c3c([2H])c([2H])c([2H])c([2H])c3c2[2H])c2c([2H])c([2H])c([2H])c([2H])c12]

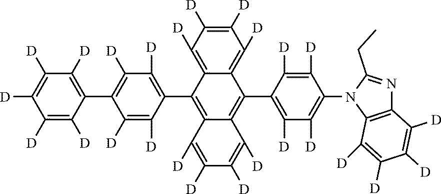 [SMILES: [2H]c1c([2H])c([2H])c(-c2c([2H])c([2H])c(-c3c4c([2H])c([2H])c([2H])c([2H])c4c(-c4c([2H])c([2H])c(-n5c(CC)nc6c([2H])c([2H])c([2H])c([2H])c65)c([2H])c4[2H])c4c([2H])c([2H])c([2H])c([2H])c34)c([2H])c2[2H])c([2H])c1[2H]]

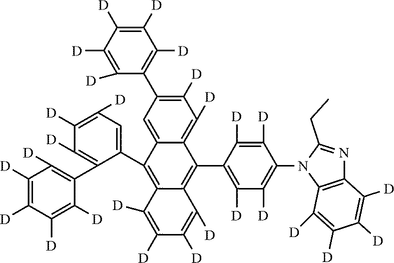 [SMILES: [2H]c1cc(-c2c3cc(-c4c([2H])c([2H])c([2H])c([2H])c4[2H])c([2H])c([2H])c3c(-c3c([2H])c([2H])c(-n4c(CC)nc5c([2H])c([2H])c([2H])c([2H])c54)c([2H])c3[2H])c3c([2H])c([2H])c([2H])c([2H])c23)c(-c2c([2H])c([2H])c([2H])c([2H])c2[2H])c([2H])c1[2H]]

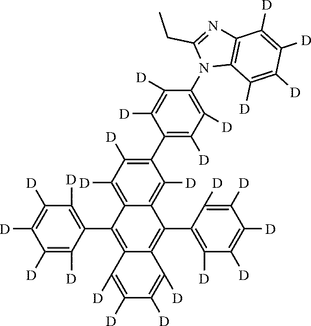 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c([2H])c(-n5c(CC)nc6c([2H])c([2H])c([2H])c([2H])c65)c([2H])c4[2H])c([2H])c([2H])c23)c([2H])c1[2H]]

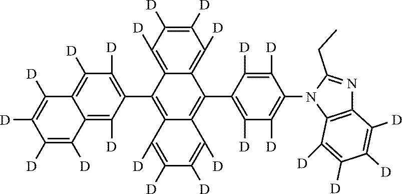 [SMILES: [2H]c1c([2H])c(-n2c(CC)nc3c([2H])c([2H])c([2H])c([2H])c32)c([2H])c([2H])c1-c1c2c([2H])c([2H])c([2H])c([2H])c2c(-c2c([2H])c([2H])c3c([2H])c([2H])c([2H])c([2H])c3c2[2H])c2c([2H])c([2H])c([2H])c([2H])c12]

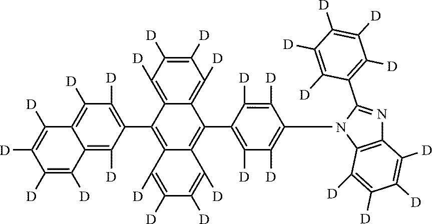 [SMILES: [2H]c1c([2H])c([2H])c(-c2nc3c([2H])c([2H])c([2H])c([2H])c3n2-c2c([2H])c([2H])c(-c3c4c([2H])c([2H])c([2H])c([2H])c4c(-c4c([2H])c([2H])c5c([2H])c([2H])c([2H])c([2H])c5c4[2H])c4c([2H])c([2H])c([2H])c([2H])c34)c([2H])c2[2H])c([2H])c1[2H]]

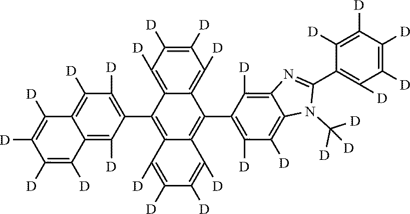 [SMILES: [2H]c1c([2H])c([2H])c(-c2nc3c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c([2H])c3n2C([2H])([2H])[2H])c([2H])c1[2H]]

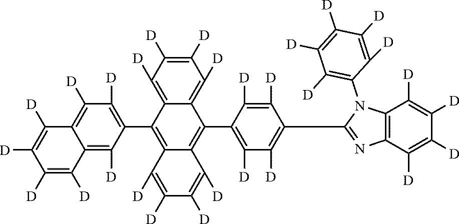 [SMILES: [2H]c1c([2H])c([2H])c(-n2c(-c3c([2H])c([2H])c(-c4c5c([2H])c([2H])c([2H])c([2H])c5c(-c5c([2H])c([2H])c6c([2H])c([2H])c([2H])c([2H])c6c5[2H])c5c([2H])c([2H])c([2H])c([2H])c45)c([2H])c3[2H])nc3c([2H])c([2H])c([2H])c([2H])c32)c([2H])c1[2H]]

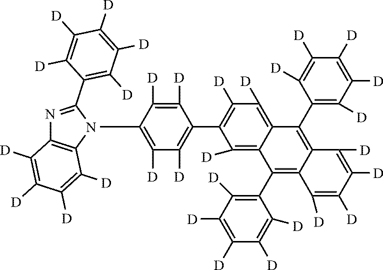 [SMILES: [2H]c1c([2H])c([2H])c(-c2c3c([2H])c([2H])c([2H])c([2H])c3c(-c3c([2H])c([2H])c([2H])c([2H])c3[2H])c3c([2H])c(-c4c([2H])c([2H])c(-n5c(-c6c([2H])c([2H])c([2H])c([2H])c6[2H])nc6c([2H])c([2H])c([2H])c([2H])c65)c([2H])c4[2H])c([2H])c([2H])c23)c([2H])c1[2H]]

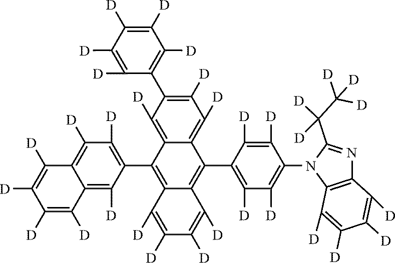 [SMILES: [2H]c1c([2H])c([2H])c(-c2c([2H])c([2H])c3c(-c4c([2H])c([2H])c(-n5c(C([2H])([2H])C([2H])([2H])[2H])nc6c([2H])c([2H])c([2H])c([2H])c65)c([2H])c4[2H])c4c([2H])c([2H])c([2H])c([2H])c4c(-c4c([2H])c([2H])c5c([2H])c([2H])c([2H])c([2H])c5c4[2H])c3c2[2H])c([2H])c1[2H]]

### Material for Organic Electroluminescence Device

The compound according to an aspect of the present invention is useful as a material for an organic EL device, and for example, is useful as an electron-transporting region for an organic EL device.

### Organic EL Device

An organic EL device according to an aspect of the present invention will be described.

The organic EL device according to an aspect of the present invention includes a cathode, an anode and one or two or more organic layers arranged between the cathode and the anode, wherein at least one layer of the organic layers includes the compound according to an aspect of the present invention (the compound represented by the formula (1)).

The organic EL device according to an aspect of the present invention preferably includes an anode, an emitting layer, an electron-transporting region, and a cathode in this order, wherein the electron-transporting region includes the compound according to an aspect of the present invention (hereinafter, the organic EL device according to the present aspect sometimes referred to as “first organic EL device”).

As a representative device configuration of the organic EL device, structures in which structures of the following (1) to (4) and the like are stacked on a substrate can be given:
(1) an anode / an emitting layer / a cathode,
(2) an anode / a hole-transporting region / an emitting layer / a cathode,
(3) an anode / an emitting layer / an electron-transporting region / a cathode,
(4) an anode / a hole-transporting region / an emitting layer / an electron-transporting region / a cathode,
wherein “/” indicates that the layers are stacked adjacent to each other.

The electron-transporting region is generally composed of one or more layer selected from an electron-injecting layer and an electron-transporting layer. The hole-transporting region is generally composed of one or more layer selected from a hole-injecting layer and a hole-transporting layer.

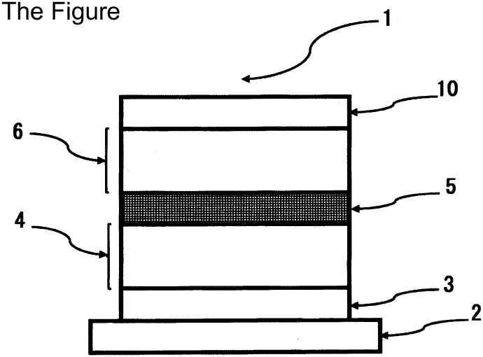

A schematic configuration of the organic EL device according to an aspect of the present invention will be described with reference to FIG. 1.

The organic EL device 1 according to an aspect of the present invention includes a substrate 2, an anode 3, an emitting layer 5, a cathode 10, a hole-transporting region 4 between the anode 3 and the emitting layer 5, and an electron-transporting region 6 between the emitting layer 5 and the cathode 10.

Members which can be used in the organic EL device according to an aspect of the present invention, materials for forming each layer, other than the above-mentioned compounds, and the like, will be described below.

### Substrate

The substrate is used as a support of an emitting device. As the substrate, glass, quartz, plastic or the like can be used, for example. Further, a flexible substrate may be used. The term “flexible substrate” means a bendable (flexible) substrate, and specific examples thereof include a plastic substrate formed of polycarbonate, polyvinyl chloride or the like.

### Anode

For the anode formed on the substrate, metals, alloys, electrically conductive compounds, mixtures thereof, and the like, which have large work function (specifically 4.0 eV or more) are preferably used.

Specific examples thereof include indium oxide-tin oxide (ITO: Indium Tin Oxide), indium oxide-tin oxide containing silicon or silicon oxide, indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), or the like.

### Hole-Injecting Layer

The hole-injecting layer is a layer containing a substance having high hole-injecting property. As the substance having high hole-injecting property, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, a polymer compound (oligomers, dendrimers, polymers, and the like), or the like can be given.

### Hole-Transporting Layer

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. Provided that a substance other than the above-described substances may be used as long as the substance has higher hole-transporting property than electron-transporting property. The layer containing the substance having high hole-transporting property may be not only a single layer, but also layers in which two or more layers formed of the above-described substances are stacked.

### Guest (Dopant) Material of Emitting Layer

The emitting layer is a layer containing a substance having high luminous property, and various materials can be used. For example, as the substance having high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for the emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for the emitting layer, aromatic amine derivatives and the like can be used. As a red fluorescent emitting material which can be used for the emitting layer, tetracene derivatives, diamine derivatives and the like can be used.

As a blue phosphorescent emitting material which can be used for the emitting layer, metal complexes such as iridium complexes, osmium complexes and platinum complexes are used. As a green phosphorescent emitting material which can be used for the emitting layer, iridium complexes and the like are used. As a red phosphorescent emitting material which can be used for the emitting layer, metal complexes such as iridium complexes, platinum complexes, terbium complexes and europium complexes are used.

### Host Material for Emitting Layer

The emitting layer may have a constitution in which the substance having high emitting property (guest material) is dispersed in another substance (host material). As a substance for dispersing the substance having high emitting property, a variety of substances can be used, and it is preferable to use a substance having a higher lowest unoccupied molecular orbital level (LUMO level) and a lower highest occupied molecular orbital level (HOMO level) than a substance having high emitting property.

As a substance (host material) for dispersing the substance having high emitting property, 1) a metal complex such as an aluminum complex, a beryllium complex, and a zinc complex, 2) a heterocyclic compound such as an oxadiazole derivative, a benzimidazole derivative, and a phenanthroline derivative, 3) a fused aromatic compound such as a carbazole derivative, an anthracene derivative, a phenanthrene derivative, a pyrene derivative, and a chrysene derivative, and 4) an aromatic amine compound such as a triarylamine derivative and a fused polycyclic aromatic amine derivative are used.

### Electron-Transporting Layer

The electron-transporting layer is a layer containing a substance having high electron-transporting property. For the electron-transporting layer, 1) a metal complex such as an aluminum complex, a beryllium complex, and a zinc complex; 2) a heteroaromatic complex such as an imidazole derivative, a benzimidazole derivative, an azine derivative, carbazole derivative, and a phenanthroline derivative; and 3) a polymer compound can be used.

In one embodiment, the electron-transporting layer may include the compound represented by the formula (1). In such a case, the electron-transporting layer may include or may not include another substance described above in addition to the compound represented by the formula (1).

In one embodiment, the electron-transporting layer includes one or more compound selected from the group consisting of a compound having an alkali metal, and a compound having a metal belonging to Group 13 of the Periodic Table of the Elements in addition to the compound represented by the formula (1). Examples of these compound include lithium fluoride, lithium oxide, 8-hydroxyquinolinolato-lithium (Liq), cesium fluoride, tris (8-quinolinolato) aluminum (Alq3), tris (4-methyl-8-quinolinolato) aluminum (Almq3), bis (2-methyl-8-quinolinolato) (4-phenylphenolato) aluminum (BAlq), and the like.

A ratio included therein (mass ratio) of the compound represented by the formula (1), and the compound having an alkali metal, and the compound having a metal belonging to Group 13 of the Periodic Table of the Elements is not particularly limited, and for example, is 10:90 to 90:10.

In the organic EL device according to an aspect of the present invention, the electron-transporting region includes a first layer (also referred to as “first electron-transporting layer” or “hole-barrier layer”) and a second layer (also referred to as “second electron-transporting layer”) in this order from the emitting layer side, and the second layer includes the compound represented by the formula (1). The configuration of the electron-transporting layer described above can be applied as the first layer. The emitting layer and the first layer may be directly in contact with each other. The first layer and the second layer may be directly in contact with each other.

In the organic EL device according to an aspect of the present invention, the electron-transporting region includes a first layer (also referred to as “first electron-transporting layer” or “hole-barrier layer”) and a second layer (also referred to as “second electron-transporting layer”) in this order from the emitting layer side, and the first layer includes the compound represented by the formula (1). The configuration of the electron-transporting layer described above can be applied as the second layer. The emitting layer and the first layer may be directly in contact with each other. The first layer and the second layer may be directly in contact with each other.

### Electron-Injecting Layer

The electron-injecting layer is a layer containing a substance having high electron-injecting property. For the electron-injecting layer, lithium (Li), ytterbium (Yb), lithium fluoride (LiF), cesium fluoride (CsF), calcium fluoride (CaF₂), a metal complex compound such as 8-hydroxyquinolinolato-lithium (Liq), an alkali metal such as lithium oxide (LiOₓ), an alkaline earth metal, or a compound thereof can be used.

### Cathode

For the cathode, metals, alloys, electrically conductive compounds, mixtures thereof, and the like, which have small work function (specifically 3.8 eV or less) are preferably used. Specific examples of such a cathode material include an element belonging to Group 1 or Group 2 of the Periodic Table of the Elements, i.e., an alkali metal such as lithium (Li) and cesium (Cs), an alkaline earth metal such as magnesium (Mg), calcium (Ca) and strontium (Sr), and an alloy containing these (e.g., MgAg and AlLi); a rare earth metal such as europium (Eu) and ytterbium (Yb), and an alloy containing these.

The cathode is usually formed by a vacuum deposition method or a sputtering method. Fur ther, when silver paste or the like is used, it is possible to use the coating method, the inkjet method or the like.

When the electron-injecting layer is provided, the cathode can be formed using various cond uctive materials such as aluminum, silver, ITO, graphene, indium oxide-tin oxide containing silicon or silicon oxide, regardless of the work function value.

In the organic EL device according to an aspect of the present invention, the thickness of each layer is not particularly limited, but is normally preferable several nm to 1 µm generally in order to suppress defects such as pinholes, to suppress applied voltages to be low, and to improve luminous efficiency.

In the organic EL device according to an aspect of the present invention, the method for forming each layer is not particularly limited. A conventionally-known method for forming each layer such as a vacuum deposition process and a spin coating process can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

An organic EL device according to other aspect of the present invention will be described.

The organic EL device according to other aspect of the present invention includes a cathode, an emitting layer, an electron-transporting region, and an anode in this order, wherein the electron-transporting region includes a compound represented by the following formula (101) (hereinafter, the organic EL device according to the present aspect also referred to as “second organic EL device”):

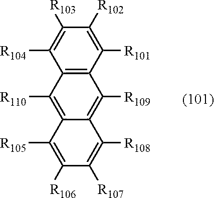

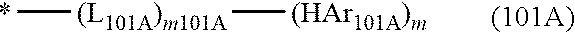

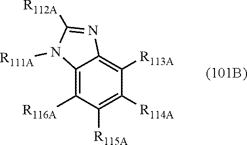

wherein in the formula (101),
at least one of R₁₀₁ to R₁₁₀ is a group represented by the formula (101A);
one or more sets of the adjacent two or more of R₁₀₁ to R₁₁₀ which are not the group represented by the formula (101A) form a substituted or unsubstituted, saturated or unsaturated ring by bonding with each other, or do not bond with each other;
R₁₀₁ to R₁₁₀ which are not the group represented by the formula (101A) and which do not bond with each other are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
in the formula (101A),
HAr_{101A} is a group represented by the formula (101B); m is an integer of 1 to 5; when two or more HAr_{101A}’s are present, the two or more HAr_{101A}’s may be the same as or different from each other;
L_{101A} is
a substituted or unsubstituted aromatic hydrocarbon group having 6 to 30 ring carbon atoms, or
a substituted or unsubstituted heterocyclic group having 5 to 30 ring atoms;
m101A is an integer of 0 to 3; when m101A is 0, m is 1 and HAr_{101A} is directly bonded with the anthracene skeleton in the formula (101) via a single bond; when m101A is 2 or 3, a plurality of L_{101A}’s is linked in series with each other and HAr_{101A} is bonded with L_{101A} which is farthest from the anthracene skeleton; when two or more L_{101A}’s are present, the two or more L_{101A}’s may be the same as or different from each other;
one or more sets of the adjacent two or more of m101A L_{101A}’s and m HAr_{101A}’s do not form a substituted or unsubstituted, saturated or unsaturated ring;
in the formula (101B),
any one of R_{111A} to R_{116A} represents a bond with L_{101A} in the formula (101A); when m101A is 0, any of carbon atoms and nitrogen atoms in the benzimidazole and any of carbon atoms in the anthracene of the formula (101) are directly bonded via a single bond;
R_{111A} to R_{116A} which do not represent a bond with L_{101A} are independently a hydrogen atom, or a substituent R; one or more sets of the adjacent two or more of R_{111A} to R_{116A} which do not represent a bond with L_{101A} do not bond with each other;
the substituent R is the same as defined in R₁₀₁ to R₁₁₀;
provided that the compound represented by the formula (101) includes at least one deuterium atom.

At least one of R₁₀₁ to R₁₁₀ in the formula (101) is a group represented by the formula (101A). The number of the group represented by the formula (101A) is not limited in R₁₀₁ to R₁₁₀, and only any one of R₁₀₁ to R₁₁₀ may be the group represented by the formula (101A), or two or more of R₁₀₁ to R₁₁₀ may be the group represented by the formula (101A). When a plurality of the groups represented by the formula (101A) is present, each of the groups represented by the formula (101A) may be the same as or different from each other.

In one embodiment, at least one of R₁₀₁, R₁₀₄, R₁₀₅, and R₁₀₈ to R₁₁₀ is the group represented by the formula (101A).

In one embodiment, at least one of R₁₀₉ and R₁₁₀ is the group represented by the formula (101A).

In one embodiment, at least one of R₁₀₉ and R₁₁₀ is the substituent R, or the group represented by the formula (101A).

The group represented by the formula (101A) will be described.

When m101A is 0, m is 1 and HAr_{101A} is directly bonded with the anthracene skeleton in the formula (101) via a single bond. When m101A is 2 or 3, a plurality of L_{101A}’s is linked in series with each other and HAr_{101A} is bonded with L_{101A} which is farthest from the anthracene skeleton.

When m is 2 or more and m101A is 1, two or more of each of HAr_{101A}’s are bonded with L_{101A}. When m is 2 or more and m101A is 2 or 3, two or more of each of HAr_{101A}’s are is bonded with L_{101A} which is farthest from the anthracene skeleton.

In one embodiment, L_{101A} is an unsubstituted aromatic hydrocarbon group having 6 to 30 ring carbon atoms, or an unsubstituted heterocyclic group having 5 to 30 ring atoms.

In one embodiment, L_{101A} is a substituted or unsubstituted aromatic hydrocarbon group having 6 to 30 ring carbon atoms.

m101A is not particularly limited, m1A may be any of 0, 1, 2 and 3, and in one embodiment, m101A is 1 or 2.

m is not particularly limited, m may be any of 1, 2, 3, 4 and 5, and in one embodiment, m is 1.

The group represented by the formula (101B) will be described.

Any one of R_{111A} to R_{116A} represents a bond with L_{101A} in the formula (101A). The expression “represents a bond” means any of carbon atoms and nitrogen atoms in the benzimidazole which R_{111A} to R_{116A} are bonded with and L_{101A} are directly bonded. When m101A is 0, any of carbon atoms and nitrogen atoms in the benzimidazole and any of carbon atoms in the anthracene of the formula (101) are directly bonded via a single bond.

In one embodiment, any one of R_{111A} to R_{113A} and R_{116A} represents a bonding position with L_{101A} in the formula (101A).

In one embodiment, any one of R_{111A} and R_{113A} to R_{116A} represents a bond with L_{101A} in the formula (101A).

In one embodiment, any one of R_{111A} and R_{113A} to R_{116A} represents a bond with L_{101A} in the formula (101A), and
R_{112A} is
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are the same as defined in the formula (101)),
a halogen atom, a cyano group, a nitro group,
an unsubstituted aryl group having 6 to 50 ring carbon atoms, or
a substituted or unsubstituted monovalent heterocyclic group having 5 to 50 ring atoms.

In one embodiment, R_{111A} represents a bonding position with L_{101A} in the formula (101A).

R_{111A} to R_{116A} which do not represent a bond with L_{101A} are independently a hydrogen atom, or a substituent R. All of R_{111A} to R_{116A} which do not represent a bond with L_{101A} may be hydrogen atoms, or part of them may be the substituent R, and for example, R_{111A} or R_{112A} may be the substituent R.

When R_{111A} represents a bonding position with L_{101A}, R_{112A} may be the substituent R. In such a case, R_{113A} to R_{116A} may be hydrogen atoms.

In one embodiment, R_{112A} represents a bonding position with L_{101A} in the formula (101A). In such a case, R_{111A} may be the substituent R. In such a case, R_{113A} to R_{116A} may be hydrogen atoms.

In one embodiment, R_{111A} to R_{116A} which do not represent a bond with L_{101A} are independently a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are the same as defined in the formula (101)),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms.

In one embodiment, the substituent R for R_{111A} to R_{116A} is a substituted or unsubstituted alkyl group having 1 to 50 carbon atoms, or a substituted or unsubstituted aryl group having 6 to 50 ring carbon atoms.

R₁₀₁ to R₁₁₀ which are not the group represented by the formula (101A) will be described.

In one embodiment, one or more sets of the adjacent two or more of R₁₀₁ to R₁₁₀ do not bond with each other.

R₁₀₁ to R₁₁₀ which are not the group represented by the formula (101A) are independently a hydrogen atom or a substituent R, and all of them may be hydrogen atoms, or part of them may be the substituent R.

In one embodiment, R₁₀₁ to R₁₀₈ are hydrogen atoms.

In one embodiment, one of R₁₀₉ and R₁₁₀ is the group represented by the formula (101A), the other of R₁₀₉ and R₁₁₀ is the substituent R, and R₁₀₁ to R₁₀₈ are hydrogen atoms.

In one embodiment, one of R₁₀₉ and R₁₁₀ is the group represented by the formula (101A), the other of R₁₀₉ and R₁₁₀ is the substituent R, R₁₀₂ or R₁₀₃ is the substituent R, and others of R₁₀₁ to R₁₁₀ are hydrogen atoms.

In one embodiment, the substituent R for R₁₀₁ to R₁₁₀ is a substituted or unsubstituted aryl group having 6 to 50 ring carbon atoms, or a substituted or unsubstituted monovalent heterocyclic group having 5 to 50 ring atoms.

The compound represented by the formula (101) includes at least one deuterium atom. Specifically, at least one hydrogen atom selected from the following hydrogen atoms in the compound represented by the formula (101) is a deuterium atom.
(101-1) Hydrogen atoms possessed by the substituted or unsubstituted, saturated or unsaturated ring formed in the case where one or more sets of the adjacent two or more of R₁₀₁ to R₁₁₀ which are not the group represented by the formula (101A) bond with each other (which include hydrogen atoms directly bonded with ring atoms in the ring, and hydrogen atoms possessed by the substituent in the case where the ring has the substituent)
(101-2) R₁₀₁ to R₁₁₀ which are hydrogen atoms in the case where R₁₀₁ to R₁₁₀ are not the group represented by the formula (101A) and do not bond with each other
(101-3) Hydrogen atoms possessed by R₁₀₁ to R₁₁₀ which are the substituents R in the case where R₁₀₁ to R₁₁₀ are not the group represented by the formula (101A) and do not bond with each other (which include hydrogen atoms possessed by the substituent in the case where the substituent R has the substituent)
(101-4) Hydrogen atoms possessed by L_{101A} (which include hydrogen atoms possessed by the substituent in the case where L_{101A} has the substituent)
(101-5) R_{111A} to R_{116A} which are hydrogen atoms in the case where R_{111A} to R_{116A} do not represent a bond with L_{101A}
(101-6) Hydrogen atoms possessed by R_{111A} to R_{116A} which are the substituents R in the case where R_{111A} to R_{116A} do not represent a bond with L_{101A} (which include hydrogen atoms possessed by the substituent in the case where the substituent R has the substituent)

Regarding the above (101-2), in one embodiment, at least one of R₁₀₁ to R₁₀₈ may be a deuterium atom, and all of R₁₀₁ to R₁₀₈ may be deuterium atoms.

Regarding the above (101-3), in one embodiment, at least one of hydrogen atoms possessed by R₁₀₁ to R₁₁₀ (for example, R₁₀₉ or R₁₁₀) which are the substituents X may be a deuterium atom, and all of hydrogen atoms possessed by R₁₀₁ to R₁₁₀ (for example, R₁₀₉ or R₁₁₀) which are the substituents X may be deuterium atoms.

Regarding the above (101-4), in one embodiment, at least one of hydrogen atoms possessed by L_{101A} may be a deuterium atom, and all of hydrogen atoms possessed by L_{101A} may be deuterium atoms.

Regarding the above (101-5), in one embodiment, at least one of R_{111A} to R_{116A} is a deuterium atom, and for example, R_{112A} is a deuterium atom.

Regarding the above (101-6), in one embodiment, at least one of hydrogen atoms possessed by R_{111A} to R_{116A} which are the substituents R in the case where R_{111A} to R_{116A} do not represent a bond with L_{101A} is a deuterium atom, and for example, at least one of hydrogen atoms possessed by R_{112A} which is the substituent R is a deuterium atom.

In one embodiment, at least one of (or all of) (101-2) “R₁₀₁ to R₁₁₀ which are hydrogen atoms in the case where R₁₀₁ to R₁₁₀ are not the group represented by the formula (101A) and do not bond with each other” is a deuterium atom, and the other hydrogen atoms included in the formula (101) are protium atoms.

In one embodiment, at least one of (or all of) (101-6) “hydrogen atoms possessed by R_{111A} to R_{116A} which are the substituents R in the case where R_{111A} to R_{116A} do not represent a bond with L_{101A}” is a deuterium atom, and the other hydrogen atoms included in the formula (101) are protium atoms.

In one embodiment, at least one of (or all of) hydrogen atoms possessed by R_{112A} which is the substituent Y is a deuterium atom, and the other hydrogen atoms included in the formula (101) are protium atoms.

In one embodiment, at least one of (or all of) (101-6) “hydrogen atoms possessed by R_{111A} to R_{116A} which are the substituents Y in the case where R_{111A} to R_{116A} do not represent a bond with L_{101A}” is a protium atom, and the other hydrogen atoms included in the formula (101) are deuterium atoms.

In one embodiment, at least one of (or all of) hydrogen atoms possessed by R_{112A} which is the substituent Y is a protium atom, and the other hydrogen atoms included in the formula (101) are deuterium atoms.

In one embodiment, at least one of (or all of) (101-6) “hydrogen atoms possessed by R_{111A} to R_{116A} which are the substituents Y in the case where R_{111A} to R_{116A} do not represent a bond with L_{101A}” is a deuterium atom, at least one of (or all of) (101-3) “hydrogen atoms possessed by R₁₀₁ to R₁₁₀ which are the substituents X in the case where R₁₀₁ to R₁₁₀ are not the group represented by the formula (1A) and do not bond with each other” is a deuterium atom, and the other hydrogen atoms included in the formula (101) are protium atoms.

In one embodiment, at least one of (or all of) hydrogen atoms possessed by R_{112A} which is the substituent Y is a deuterium atom, at least one of (or all of) hydrogen atoms possessed by R₁₁₀ which is the substituent X is a deuterium atom, and the other hydrogen atoms included in the formula (101) are protium atoms.

In one embodiment, all of hydrogen atoms included in the formula (101) are deuterium atoms.

In one embodiment, the compound represented by the formula (101) is the compound represented by the following formula (111):

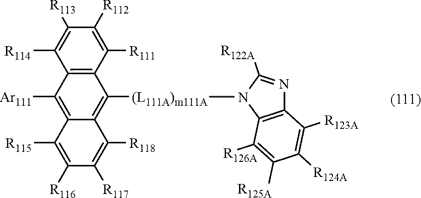

wherein in the formula (111),
one or more sets of the adjacent two or more of R₁₁₁ to R₁₁₈ form a substituted or unsubstituted, saturated or unsaturated ring by bonding with each other, or do not bond with each other;
R₁₁₁ to R₁₁₈ which do not bond with each other are independently a hydrogen atom or a substituent R;
the substituent R is the same as defined in the formula (101);
L_{111A} is
a substituted or unsubstituted aromatic hydrocarbon group having 6 to 30 ring carbon atoms, or
a substituted or unsubstituted heterocyclic group having 5 to 30 ring atoms;
m111A is an integer of 0 to 3; when m111A is 0, any of carbon atoms and nitrogen atoms in the benzimidazole and any of carbon atoms in the anthracene are directly bonded via a single bond; when two or more L_{111A}’s are present, the two or more L_{111A}’s may be the same as or different from each other;
one or more sets of the adjacent two or more of m111A L_{111A}’s do not form a substituted or unsubstituted, saturated or unsaturated ring;
R_{122A} to R_{126A} are independently a hydrogen atom or a substituent R;
the substituent R is the same as defined in the formula (101);
Ar₁₁₁ is
a substituted or unsubstituted aryl group having 6 to 30 ring carbon atoms, or
a substituted or unsubstituted monovalent heterocyclic group having 5 to 30 ring atoms;
provided that the compound represented by the formula (111) includes at least one deuterium atom.

In one embodiment, one or more sets of the adjacent two or more of R₁₁₁ to R₁₁₈ do not bond with each other.

R₁₁₁ to R₁₁₈ are independently a hydrogen atom or a substituent R, and all of them may be hydrogen atoms, or part of them may be the substituent R.

In one embodiment, R₁₁₁ to R₁₁₈ are hydrogen atoms.

In one embodiment, R₁₁₂ or R₁₁₃ is the substituent R, and others of R₁₁₁ to R₁₁₈ are hydrogen atoms.

When m111A is 0, the benzimidazole skeleton is directly bonded with the anthracene skeleton in the formula (111) via a single bond. When m111A is 2 or 3, a plurality of L_{111A}’s is linked in series with each other and the benzimidazole skeleton is bonded with L_{111A} which is farthest from the anthracene skeleton.

In one embodiment, L_{111A} is a substituted or unsubstituted aromatic hydrocarbon group having 6 to 30 ring carbon atoms.

m111A is not particularly limited, m111A may be any of 0, 1, 2 and 3, and in one embodiment, m111A is 1 or 2.

The compound represented by the formula (111) includes at least one deuterium atom. Specifically, at least one hydrogen atom selected from the following hydrogen atoms in the compound represented by the formula (111) is a deuterium atom.
(111-1) Hydrogen atoms possessed by the substituted or unsubstituted, saturated or unsaturated ring formed in the case where one or more sets of the adjacent two or more of R₁₁₁ to R₁₁₈ bond with each other (which include hydrogen atoms directly bonded with ring atoms in the ring, and hydrogen atoms possessed by the substituent in the case where the ring has the substituent)
(111-2) R₁₁₁ to R₁₁₈ which are hydrogen atoms in the case where R₁₁₁ to R₁₁₈ do not bond with each other
(111-3) Hydrogen atoms possessed by R₁₁₁ to R₁₁₈ which are the substituents R in the case where R₁₁₁ to R₁₁₈ do not bond with each other (which include hydrogen atoms possessed by the substituent in the case where the substituent R has the substituent)
(111-4) Hydrogen atoms possessed by Ar₁₁₁ (which include hydrogen atoms possessed by the substituent in the case where Ar₁₁₁ has the substituent)
(111-5) Hydrogen atoms possessed by L_{111A} (which include hydrogen atoms possessed by the substituent in the case where L_{111A} has the substituent)
(111-6) R_{122A} to R_{126A} which are hydrogen atoms in R_{122A} to R_{126A}
(111-7) Hydrogen atoms possessed by R_{122A} to R_{126A} which are the substituents R in R_{122A} to R_{126A} (which include hydrogen atoms possessed by the substituent in the case where the substituent R has the substituent)

The compound represented by the formula (101) can be synthesized in accordance with Examples by using known alternative reactions or raw materials adapted to the target compound.

Specific examples of the compound represented by the formula (101) include those described as the compound represented by the formula (1).

As the structure of each moiety in the compound represented by the formula (101), each structure described in the corresponding moiety of the compound represented by the formula (1) may appropriately be applied.

The second organic EL device is the same as the first organic EL device, except that the electron-transporting region includes the compound represented by the formula (101). Accordingly, as the configuration other than the compound represented by the formula (101) in the second organic EL device, the configuration described in the first organic EL device can be applied.

### Electrical Apparatus

An electronic apparatus according to an aspect of the present invention is characterized by including the organic EL device according to an aspect of the present invention (which includes the first organic EL device and the second organic EL device).

Specific examples of the electronic apparatus include display components such as an organic EL panel module; display devices for a television, a cellular phone and a personal computer; and emitting devices such as a light and a vehicular lamp; and the like.

### EXAMPLES

### Compound

Compounds represented by the formula (1) used in the fabrication of the organic EL devices of Examples are shown below.

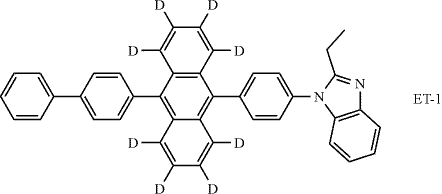 [SMILES: [2H]c1c([2H])c([2H])c2c(-c3ccc(-n4c(CC)nc5ccccc54)cc3)c3c([2H])c([2H])c([2H])c([2H])c3c(-c3ccc(-c4ccccc4)cc3)c2c1[2H]]

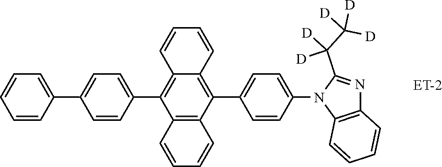 [SMILES: [2H]C([2H])([2H])C([2H])([2H])c1nc2ccccc2n1-c1ccc(-c2c3ccccc3c(-c3ccc(-c4ccccc4)cc3)c3ccccc23)cc1]

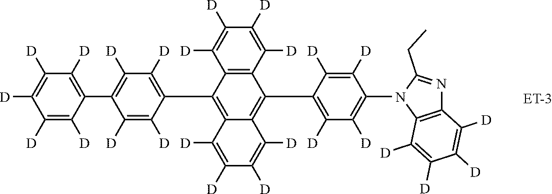 [SMILES: [2H]c1c([2H])c([2H])c(-c2c([2H])c([2H])c(-c3c4c([2H])c([2H])c([2H])c([2H])c4c(-c4c([2H])c([2H])c(-n5c(CC)nc6c([2H])c([2H])c([2H])c([2H])c65)c([2H])c4[2H])c4c([2H])c([2H])c([2H])c([2H])c34)c([2H])c2[2H])c([2H])c1[2H]]

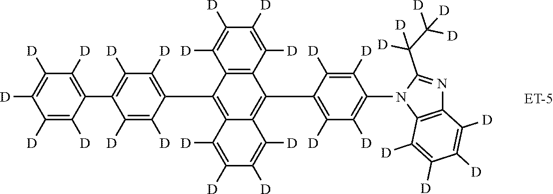 [SMILES: [2H]c1c([2H])c([2H])c(-c2c([2H])c([2H])c(-c3c4c([2H])c([2H])c([2H])c([2H])c4c(-c4c([2H])c([2H])c(-n5c(C([2H])([2H])C([2H])([2H])[2H])nc6c([2H])c([2H])c([2H])c([2H])c65)c([2H])c4[2H])c4c([2H])c([2H])c([2H])c([2H])c34)c([2H])c2[2H])c([2H])c1[2H]]

A comparative compound used in the fabrication of the organic EL devices of Comparative Example is shown below.

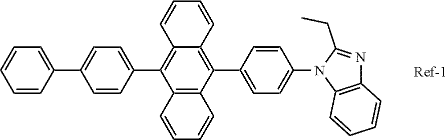 [SMILES: CCc1nc2ccccc2n1-c1ccc(-c2c3ccccc3c(-c3ccc(-c4ccccc4)cc3)c3ccccc23)cc1]

Other compounds used in the fabrication of the organic EL devices of Examples and Comparative Example are shown below.

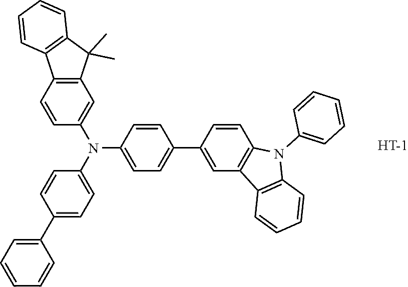 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

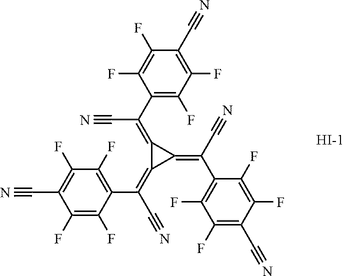 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

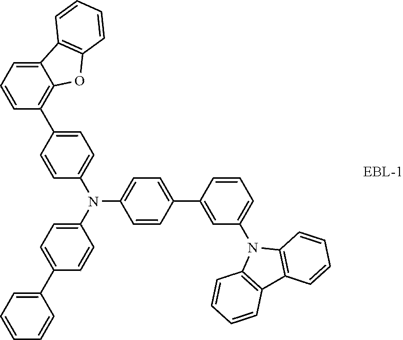 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

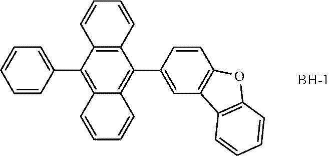 [SMILES: c1ccc(-c2c3ccccc3c(-c3ccc4oc5ccccc5c4c3)c3ccccc23)cc1]

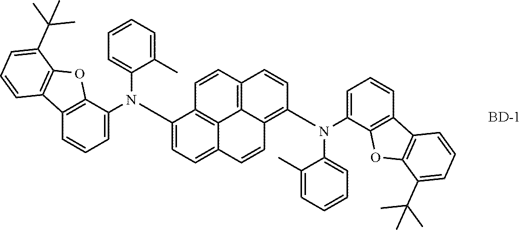 [SMILES: Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

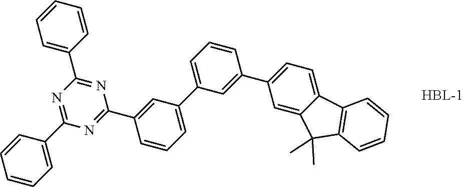 [SMILES: CC1(C)c2ccccc2-c2ccc(-c3cccc(-c4cccc(-c5nc(-c6ccccc6)nc(-c6ccccc6)n5)c4)c3)cc21]

### Fabrication of Organic EL Device

Organic EL devices were fabricated as follows.

### Example 1

A 25 mm × 75 mm × 1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The ITO has the film thickness of 130 nm.

The glass substrate with the transparent electrode after being cleaned was mounted onto a substrate holder in a vacuum vapor deposition apparatus. First, compounds HT-1 and HI-1 were co-deposited on the surface on the side where the transparent electrode was formed so as to cover the transparent electrode to be 3% by mass in a proportion of the compound HI-1 to form a first hole-transporting layer having the thickness of 10 nm.

A compound HT-1 was deposited on the first hole-transporting layer to form a second hole-transporting layer having the thickness of 80 nm.

A compound EBL-1 was deposited on the second hole-transporting layer to form a third hole-transporting layer (also referred to as “electron-barrier layer”) having the thickness of 5 nm.

A compound BH-1 (host material) and a compound BD-1 (dopant material) were co-deposited on the third hole-transporting layer to be 4% by mass in a proportion of the compound BD-1 to form an emitting layer having the thickness of 25 nm.

A compound HBL-1 was deposited on the emitting layer to form a first electron-transporting layer (also referred to as “hole-barrier layer”) having the thickness of 5 nm.

A compound ET-1 was deposited on the first electron-transporting layer to form a second electron-transporting layer having the thickness of 20 nm.

A metal Yb was deposited on the second electron-transporting layer to form an electron-injecting layer having the thickness of 1 nm.

A metal Al was deposited on the electron-injecting layer to form a cathode having the thickness of 50 nm.

The device configuration of the organic EL device of Example 1 is schematically shown as follows. ITO(130)/HT-1:HI-1(10:3%)/HT-1(80)/EBL-1(5)/BH-1:BD-1(25:4%)/HBL-1(5)/ET-1(20)/Yb(1)/Al(50)

The numerical values in parentheses indicate the film thickness (unit: nm). The numerical values represented by percent in parentheses indicate a proportion (% by mass) of the latter compound in the layer.

### Examples 2 to 4

Organic EL devices were fabricated in the same manner as in Example 1, except that compounds shown in Table 1 were used instead of the compound ET-1 in formation of the second electron-transporting layer.

### Comparative Example 1

Organic EL devices were fabricated in the same manner as in Example 1, except that the compound Ref-1 was used instead of the compound ET-1 in formation of the second electron-transporting layer.

### Evaluation of Organic EL Device

Regarding the organic EL devices fabricated in Examples 1 to 4 and Comparative Example 1, device lifetime was evaluated as follows. The results are shown in Table 1.

### Device Lifetime

Regarding the obtained organic EL device, a voltage was applied to the organic EL device at room temperature so that the current density became 50 mA/cm², and the time until the luminance became 95% of the initial luminance (LT95 (unit: h)) was measured. The device lifetime is a relative value when the value of Comparative Example 1 is 100.

TABLE 1

| | Second electron-transporting layer | LT95 (Relative value when value of Comparative Example 1 is 100) |
|---|---|---|
| Example 1 | ET-1 | 117 |
| Example 2 | ET-2 | 178 |
| Example 3 | ET-3 | 132 |
| Example 4 | ET-5 | 183 |
| Comparative Example 1 | Ref-1 | 100 |

### Synthesis of Compound

### (Synthesis Example 1) Synthesis of ET-1

The compound ET-1 was synthesized through the synthetic route described below.

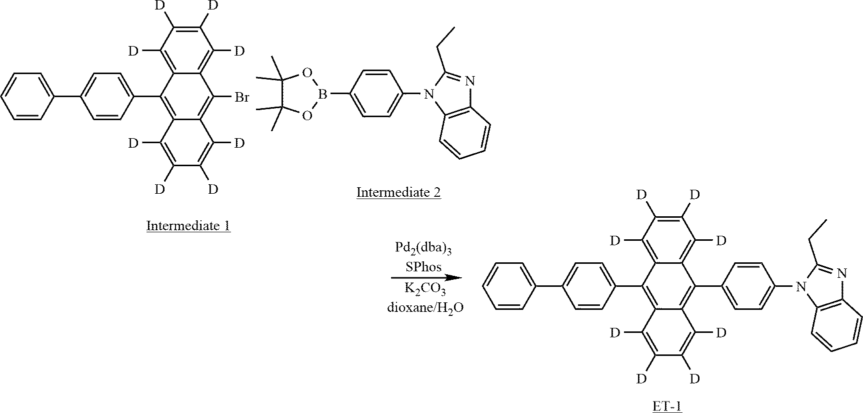

Dioxane (30 mL) and water (6 mL) were added to Intermediate 1 (2.5 g, 6.0 mmol), Intermediate 2 (2.3 g, 6.6 mmol), Pd₂(dba)₃ (110 mg, 0.12 mmol), SPhos (196 mg, 0.48 mmol), and potassium carbonate (1.7 g, 12 mmol) under an argon atmosphere, and it was stirred at 100° C. for 7 hours. After reaction, the reaction solution was purified by short pass silica gel column chromatography using toluene as mobile phase. The solution was concentrated, and the obtained residue was purified by column chromatography to obtain a pale yellow solid (2.1 g, 63% yield). The obtained solid was ET-1 being an intended product, and the mass spectrum thereof was analyzed as m/e = 559 for a molecular weight of 558.

### (Synthesis Example 2) Synthesis of ET-2

The compound ET-2 was synthesized through the synthetic route described below.

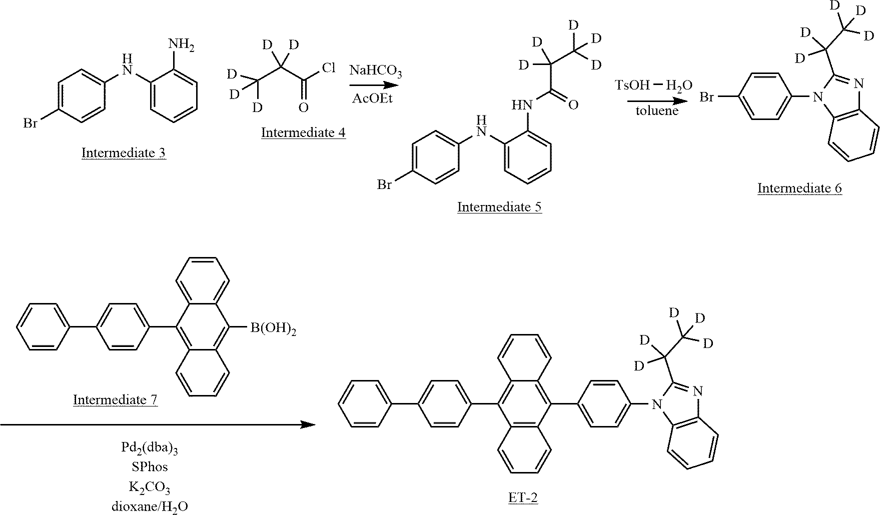

### 1) Synthesis of Intermediate 5

Intermediate 3 (15 g, 57 mmol) and sodium hydrogen carbonate (7.2 g, 86 mmol) were suspended in ethyl acetate (500 mL) under an argon atmosphere, and Intermediate 4 (5.56 g, 57 mmol) was added dropwise thereto at 0° C. The reaction solution was stirred at room temperature for two hours, subsequently, water was added thereto, and then it was extracted with ethyl acetate. The organic phase was concentrated, and the obtained residue was purified by silica gel column chromatography to obtain a white solid (16.5 g). The obtained solid was Intermediate 5 being an intended product, and the mass spectrum thereof was analyzed as m/e = 325 for a molecular weight of 324.

### 2) Synthesis of Intermediate 6

A tosylic acid monohydrate (4.4 g, 23 mmol) were added to a toluene solution (300 mL) of Intermediate 5 (15 g, 46 mmol) at room temperature under an argon atmosphere, and it was heated and stirred at 100° C. for 20 hours. After reaction, water was added to the reaction solution to separate the solution, and then the organic phase was concentrated. The obtained residue was purified by column chromatography to obtain a white solid (11.6 g, 82% yield). The obtained solid was Intermediate 6 being an intended product, and the mass spectrum thereof was analyzed as m/e = 307 for a molecular weight of 306.

### 3) Synthesis of ET-2

Dioxane (100 mL) and water (20 mL) were added to Intermediate 6 (10.0 g, 16.3 mmol), Intermediate 7 (6.42 g, 17.1 mmol), Pd₂(dba)₃ (300 mg, 0.327 mmol), SPhos (536 mg, 1.31 mmol), and potassium carbonate (4.51 g, 32.7 mmol) under an argon atmosphere, and it was stirred at 100° C. for 7 hours. After reaction, the reaction solution was purified by short pass silica gel column chromatography using toluene as mobile phase. The solution was concentrated, and the obtained residue was purified by column chromatography to obtain a pale yellow solid (7.11 g, 78% yield). The obtained solid was ET-2 being an intended product, and the mass spectrum thereof was analyzed as m/e = 556 for a molecular weight of 555.

### (Synthesis Example 3) Synthesis of ET-3

The compound ET-3 was synthesized through the synthetic route described below.

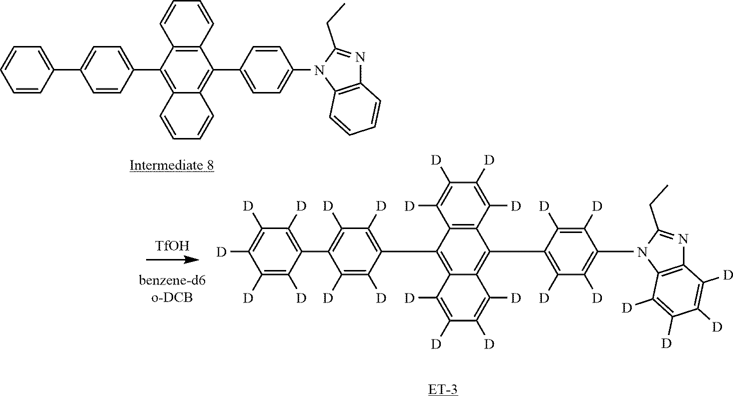

Dichlorobenzene (280 mL) were added to Intermediate 8 (7.0 g, 12.7 mmol) under an argon atmosphere, and it was heated and dissolved at 80° C., and then benzene-d6 (140 mL) and TfOH (5.62 mL, 63.6 mmol) were added thereto, and it was stirred at 70° C. for 11 hours. After reaction, it was naturally cooled, heavy water (150 mL) was added thereto, and it was stirred at room temperature for an hour. The obtained solid was collected by filtration. The obtained solid was dissolved in cyclohexanone, and it was purified by column chromatography using toluene as mobile phase to obtain a pale yellow solid (4.2 g, 57% yield). The obtained solid was ET-3 being an intended product, and the mass spectrum thereof was analyzed as m/e = 576 for a molecular weight of 575.

### (Synthesis Example 4) Synthesis of ET-4

The compound ET-4 was synthesized through the synthetic route described below.

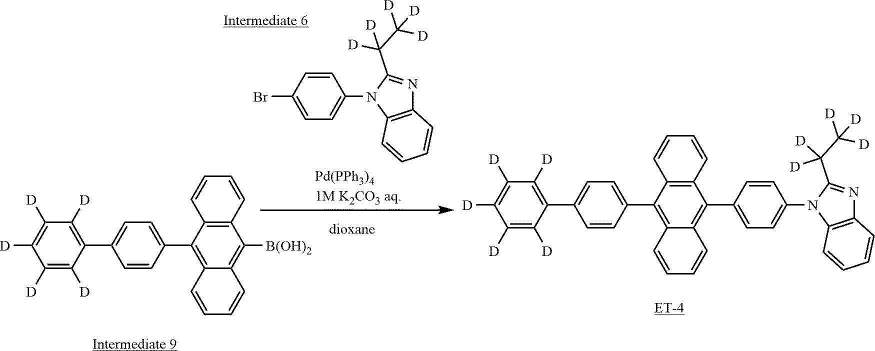

Intermediate 6 (5.7 g, 18.6 mmol), Intermediate 9 (7.1 g, 18.6 mmol), Pd(PPh₃)₄ (645 mg, 0.558 mmol), aqueous solution of potassium carbonate (1 M, 55.8 mL, 55.8 mmol), and dioxane (93 mL) were mixed under an argon atmosphere, and it was stirred at 90° C. for 19 hours. After reaction, it was naturally cooled, and the obtained solid was collected by filtration. It was purified by short pass silica gel column chromatography using toluene as mobile phase to obtain a pale yellow solid (6.6 g, 63% yield). The obtained solid was ET-4 being an intended product, and the mass spectrum thereof was analyzed as m/e = 561 for a molecular weight of 560.

### (Synthesis Example 5) Synthesis of ET-5

The compound ET-5 was synthesized through the synthetic route described below.

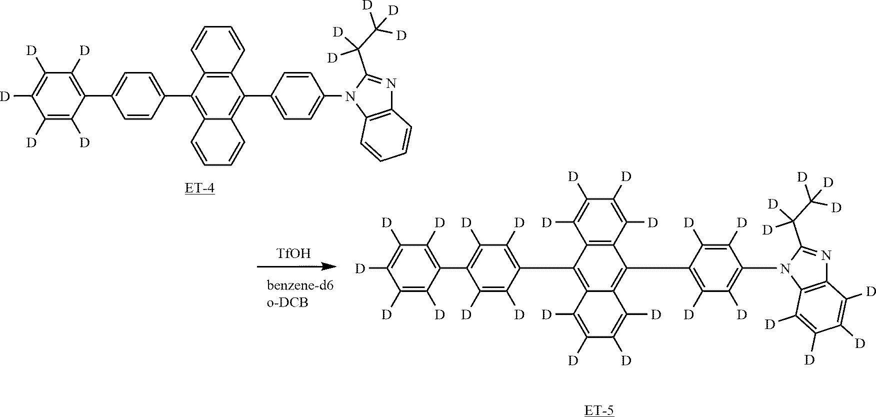

Dichlorobenzene (244 mL) were added to ET-4 (6.1 g, 10.9 mmol) under an argon atmosphere, and it was heated and dissolved at 120° C., and then benzene-d6 (61 mL) and TfOH (4.81 mL, 54.4 mmol) were added thereto, and it was stirred at 80° C. for 6 hours. After reaction, it was naturally cooled, heavy water (100 mL) was added thereto, and it was stirred at room temperature for an hour. The obtained solid was collected by filtration. The obtained solid was dissolved in cyclohexanone, and it was purified by column chromatography using toluene as mobile phase to obtain a pale yellow solid (4.2 g, 57% yield). The obtained solid was ET-5 being an intended product, and the mass spectrum thereof was analyzed as m/e = 581 for a molecular weight of 580.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the specification of Japanese application(s) on the basis of which the present application claims Paris convention priority are incorporated herein by reference in its entirety.

1. A compound represented by the following formula (1): 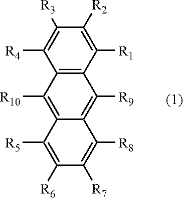 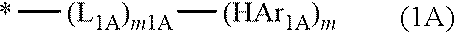 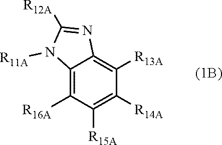
wherein in the formula (1),
at least one of R₁ to R₁₀ is a group represented by the formula (1A);
one or more sets of the adjacent two or more of R₁ to R₁₀ which are not the group represented by the formula (1A) form a substituted or unsubstituted, saturated or unsaturated ring by bonding with each other, or do not bond with each other;
R₁ to R₁₀ which are not the group represented by the formula (1A) and which do not bond with each other are independently a hydrogen atom or a substituent X;
the substituent X is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₅ are present, the two or more of each of R₉₀₁ to R₉₀₅ may be the same as or different from each other;
when two or more substituents X are present, the two or more substituents X may be the same as or different from each other;
in the formula (1A),
HAr_{1A} is a group represented by the formula (1B); m is an integer of 1 to 5; when two or more HAr_{1A}’s are present, the two or more HAr_{1A}’s may be the same as or different from each other;
L_{1A} is
a substituted or unsubstituted aromatic hydrocarbon group having 6 to 30 ring carbon atoms, or
a substituted or unsubstituted heterocyclic group having 5 to 30 ring atoms;
m1A is an integer of 0 to 3; when m1A is 0, m is 1 and HAr_{1A} is directly bonded with the anthracene skeleton in the formula (1) via a single bond; when m1A is 2 or 3, a plurality of L_{1A}’s is linked in series with each other and HAr_{1A} is bonded with L_{1A} which is farthest from the anthracene skeleton; when two or more L_{1A}’s are present, the two or more L_{1A}’s may be the same as or different from each other;
one or more sets of the adjacent two or more of m1A L_{1A}’s and m HAr_{1A}’s do not form a substituted or unsubstituted, saturated or unsaturated ring;
in the formula (1B),
any one of R_{11A} to R_{16A} represents a bond with L_{1A} in the formula (1A); when m1A is 0, any of carbon atoms and nitrogen atoms in the benzimidazole and any of carbon atoms in the anthracene of the formula (1) are directly bonded via a single bond;
R_{11A} to R_{16A} which do not represent a bond with L_{1A} are independently a hydrogen atom or a substituent Y; one or more sets of the adjacent two or more of R_{11A} to R_{16A} which do not represent a bond with L_{1A} do not bond with each other;
the substituent Y is
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom,
a cyano group,
a nitro group,
a substituted or unsubstituted aryl group having 6 to 50 ring carbon atoms, or
a substituted or unsubstituted monovalent heterocyclic group having 5 to 50 ring atoms;
R₉₀₁ to R₉₀₅ are the same as defined in the formula (1);
R₉₀₆ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₆ to R₉₀₇ are present, the two or more of each of R₉₀₆ to R₉₀₇ may be the same as or different from each other;
when two or more substituents Y are present, the two or more substituents Y may be the same as or different from each other;
provided that the compound represented by the formula (1) satisfies all of the following conditions 1 to 6;
condition 1: the compound represented by the formula (1) comprises at least one deuterium atom;
condition 2: at least one of R₉ and R₁₀ is the group represented by the formula (1A) or the substituent X;
condition 3: when at least one of R₁ to R₈ in the formula (1) is the substituent X, m is 1 in the formula (1A) and the substituent in the case of “substituted or unsubstituted” wherein L_{1A} is a substituted or unsubstituted phenyl group is not a substituted or unsubstituted benzimidazolyl group;
condition 4: when at least one of R₁ to R₈ in the formula (1) is a substituted or unsubstituted naphthyl group, R_{12A} in the formula (1B) do not represent a bond with L_{1A} in the formula (1A);
condition 5: when at least one of R₂, R₃, R₆, and R₇ in the formula (1) is the group represented by the formula (1A), at least one hydrogen atom selected from the group consisting of, R₁ to R₁₀ which are hydrogen atoms, hydrogen atoms possessed by the substituted or unsubstituted, saturated or unsaturated ring formed in the case where one or more sets of the adjacent two or more of R₁ to R₁₀ bond with each other, hydrogen atoms possessed by R₁ to R₁₀ which are the substituents X, and hydrogen atoms possessed by L_{1A} is a deuterium atom; and
condition 6: when any one of R_{14A} and R_{15A} in the formula (1B) represents a bond with L_{1A} in the formula (1A), m1A is not 0.

2. The compound according to claim 1, wherein any one of R_{11A} to R_{13A} and R_{16A} in the formula (1B) represents a bonding position with L_{1A} in the formula (1A).

3. The compound according to claim 1, wherein R_{11A} in the formula (1B) represents a bonding position with L_{1A} in the formula (1A).

4. The compound according to claim 1, wherein at least one of R₁, R₄, R₅, and R₈ to R₁₀ in the formula (1) is the group represented by the formula (1A).

5. The compound according to claim 1, wherein at least one of R₉ and R₁₀ in the formula (1) is the group represented by the formula (1A).

6. The compound according to claim 1, wherein one or more sets of the adjacent two or more of R₁ to R₁₀ in the formula (1) do not bond with each other.

7. The compound according to claim 1, wherein R₁ to R₈ in the formula (1) are hydrogen atoms.

8. The compound according to claim 1, wherein at least one of R₁ to R₈ in the formula (1) is a deuterium atom.

9. The compound according to claim 1, wherein all of R₁ to R₈ in the formula (1) are deuterium atoms.

10. The compound according to claim 1, wherein at least one of hydrogen atoms possessed by R_{11A} to R_{16A} which do not represent a bond with L_{1A} and which are the substituents Y in the formula (1B) is a deuterium atom.

11. The compound according to claim 1, wherein at least one of hydrogen atoms possessed by R_{12A} which are the substituents Y in the formula (1B) is a deuterium atom.

12. The compound according to claim 1, wherein the compound represented by the formula (1) is the compound represented by the following formula (11): 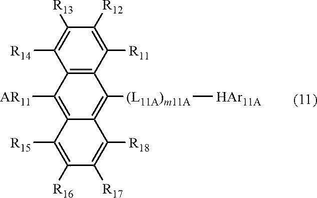 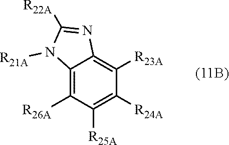
wherein in the formula (11),
one or more sets of the adjacent two or more of R₁₁ to R₁₈ form a substituted or unsubstituted, saturated or unsaturated ring by bonding with each other, or do not bond with each other;
R₁₁ to R₁₈ which do not bond with each other are independently a hydrogen atom or a substituent X;
the substituent X is the same as defined in the formula (1);
HAr_{11A} is a group represented by the formula (11B);
L_{11A} is
a substituted or unsubstituted aromatic hydrocarbon group having 6 to 30 ring carbon atoms, or
a substituted or unsubstituted heterocyclic group having 5 to 30 ring atoms;
m11A is an integer of 0 to 3; when m11A is 0, HAr_{11A} is directly bonded with the anthracene skeleton in the formula (11) via a single bond; when m11A is 2 or 3, a plurality of L_{11A}’s is linked in series with each other and HAr_{11A} is bonded with L_{11A} which is farthest from the anthracene skeleton; when two or more L_{11A}’s are present, the two or more L_{11A}’s may be the same as or different from each other;
one or more sets of the adjacent two or more of m11A L_{11A}’s and HAr_{11A} do not form a substituted or unsubstituted, saturated or unsaturated ring;
in the formula (11B),
any one of R_{21A} to R_{26A} represents a bond with L_{11A} in the formula (11); when m11A is 0, any of carbon atoms and nitrogen atoms in the benzimidazole and any of carbon atoms in the anthracene of the formula (11) are directly bonded via a single bond;
R_{21A} to R_{26A} which do not represent a bond with L_{11A} are independently a hydrogen atom or a substituent Y; one or more sets of the adjacent two or more of R_{21A} to R_{26A} do not bond with each other;
the substituent Y is the same as defined in the formula (1B);
Ar₁₁ is
a substituted or unsubstituted aryl group having 6 to 30 ring carbon atoms, or
a substituted or unsubstituted monovalent heterocyclic group having 5 to 30 ring atoms;
provided that the compound represented by the formula (11) comprises at least one deuterium atom; when at least one of R₁₁ to R₁₈ is the substituent X, the substituent in the case of “substituted or unsubstituted” wherein L_{11A} is a substituted or unsubstituted phenyl group is not a substituted or unsubstituted benzimidazolyl group.

13. The compound according to claim 12, wherein the compound represented by the formula (11) is the compound represented by the following formula (12): 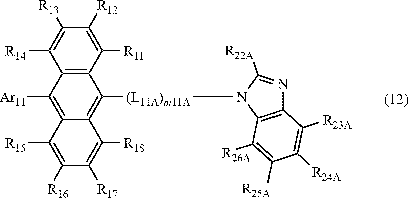
wherein in the formula (12),
R₁₁ to R₁₈, L_{11A}, m11A, and Ar₁₁ are the same as defined in the formula (11);
R_{22A} to R_{26A} are independently a hydrogen atom or a substituent Y; one or more sets of the adjacent two or more of R_{22A} to R_{26A} do not bond with each other;
the substituent Y is the same as defined in the formula (1B);
provided that the compound represented by the formula (12) comprises at least one deuterium atom; when at least one of R₁₁ to R₁₈ is the substituent X, the substituent in the case of “substituted or unsubstituted” wherein L_{11A} is a substituted or unsubstituted phenyl group is not a substituted or unsubstituted benzimidazolyl group.

14. The compound according to claim 12, wherein m11A is 1.

15. The compound according to claim 12, wherein the compound represented by the formula (11) is the compound represented by the following formula (13): 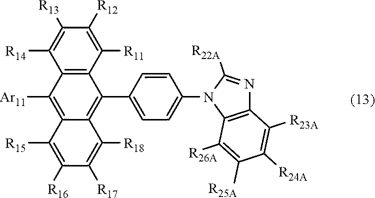
wherein in the formula (13),
R₁₁ to R₁₈, R_{22A} to R_{26A}, and Ar₁₁ are the same as defined in the formula (11);
provided that the compound represented by the formula (13) comprises at least one deuterium atom.

16. The compound according to claim 12, wherein the compound represented by the formula (11) is the compound represented by the following formula (14): 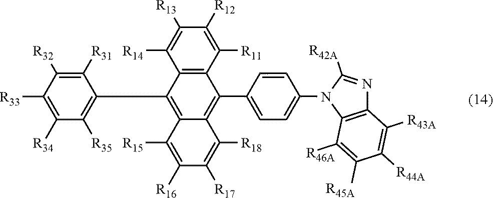
wherein in the formula (14),
R₁₁ to R₁₈ are the same as defined in the formula (11);
one or more sets of the adjacent two or more of R₃₁ to R₃₅ form a substituted or unsubstituted, saturated or unsaturated ring by bonding with each other, or do not bond with each other;
R₃₁ to R₃₅ which do not bond with each other are independently
a hydrogen atom,
a substituted or unsubstituted alkyl group having 1 to 50 carbon atoms,
a substituted or unsubstituted aryl group having 6 to 50 ring carbon atoms, or
a substituted or unsubstituted monovalent heterocyclic group having 5 to 50 ring atoms;
R_{42A} to R_{46A} are independently
a hydrogen atom, or
a substituted or unsubstituted alkyl group having 1 to 50 carbon atoms;
provided that the compound represented by the formula (14) comprises at least one deuterium atom.

17. The compound according to claim 16, wherein the compound represented by the formula (14) is the compound represented by the following formula (15): 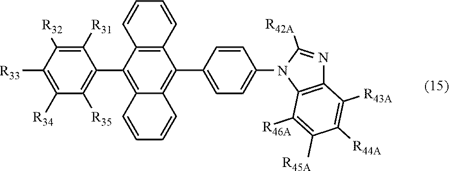
wherein in the formula (15),
R₃₁ to R₃₅ and R_{42A} to R_{46A} are the same as defined in the formula (14);
provided that the compound represented by the formula (15) comprises at least one deuterium atom.

18. The compound according to claim 1, wherein the substituent in the case of “substituted or unsubstituted”, the substituent X, and the substituent Y are a group selected from the group consisting of
an alkyl group having 1 to 50 carbon atoms,
an aryl group having 6 to 50 ring carbon atoms, and
a monovalent heterocyclic group having 5 to 50 ring atoms.

19. The compound according to claim 1, wherein the substituent in the case of “substituted or unsubstituted”, the substituent X, and the substituent Y are a group selected from the group consisting of
an alkyl group having 1 to 18 carbon atoms,
an aryl group having 6 to 18 ring carbon atoms, and
a monovalent heterocyclic group having 5 to 18 ring atoms.

20. The compound according to claim 1, which is a material for an organic electroluminescence device.

21. An organic electroluminescence device comprising
a cathode,
an anode, and
one or two or more organic layers arranged between the cathode and the anode,
wherein at least one layer of the organic layers comprises the compound according to claim 1.

22. The organic electroluminescence device according to claim 21, which comprises an anode, an emitting layer, an electron-transporting region, and a cathode in this order,
wherein the electron-transporting region comprises the compound.

23. The organic electroluminescence device according to claim 22, wherein the electron-transporting region comprises a first layer and a second layer in this order from the emitting layer side, and
the second layer comprises the compound.

24. An organic electroluminescence device comprising a cathode,
an emitting layer,
an electron-transporting region, and
an anode in this order,
wherein the electron-transporting region comprises a compound represented by the following formula (101): 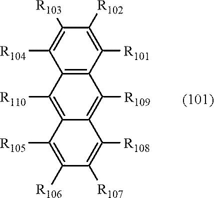 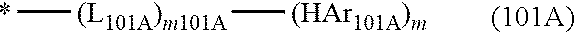 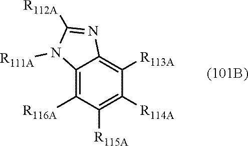
wherein in the formula (101),
at least one of R₁₀₁ to R₁₁₀ is a group represented by the formula (101A);
one or more sets of the adjacent two or more of R₁₀₁ to R₁₁₀ which are not the group represented by the formula (101A) form a substituted or unsubstituted, saturated or unsaturated ring by bonding with each other, or do not bond with each other;
R₁₀₁ to R₁₁₀ which are not the group represented by the formula (101A) and which do not bond with each other are independently a hydrogen atom or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀ ₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
when two or more substituents R are present, the two or more substituents R may be the same as or different from each other;
in the formula (101A),
HAr_{101A} is a group represented by the formula (101B); m is an integer of 1 to 5; when two or more HAr_{101A}’s are present, the two or more HAr_{101A}’s may be the same as or different from each other;
L_{101A} is
a substituted or unsubstituted aromatic hydrocarbon group having 6 to 30 ring carbon atoms, or
a substituted or unsubstituted heterocyclic group having 5 to 30 ring atoms;
m101A is an integer of 0 to 3; when m101A is 0, m is 1 and HAr_{101A} is directly bonded with the anthracene skeleton in the formula (101) via a single bond; when m101A is 2 or 3, a plurality of L_{101A}’s is linked in series with each other and HAr_{101A} is bonded with L_{101A} which is farthest from the anthracene skeleton; when two or more L_{101A}’s are present, the two or more L_{101A}’s may be the same as or different from each other;
one or more sets of the adjacent two or more of m101A L_{101A}’s and m HAr_{101A}’s do not form a substituted or unsubstituted, saturated or unsaturated ring;
in the formula (101B),
any one of R_{111A} to R_{116A} represents a bond with L_{101A} in the formula (101A); when m101A is 0, any of carbon atoms and nitrogen atoms in the benzimidazole and any of carbon atoms in the anthracene of the formula (101) are directly bonded via a single bond;
R_{111A} to R_{116A} which do not represent a bond with L_{101A} are independently a hydrogen atom, or a substituent R; one or more sets of the adjacent two or more of R_{111A} to R_{116A} which do not represent a bond with L_{101A} do not bond with each other;
the substituent R is the same as defined in R₁₀₁ to R₁₁₀;
provided that the compound represented by the formula (101) comprises at least one deuterium atom.

25. The organic electroluminescence device according to claim 24, wherein the electron-transporting region comprises a first layer and a second layer in this order from the emitting layer side, and the second layer comprises the compound.

26. The organic electroluminescence device according to claim 24, wherein R_{111A} to R_{116A} which do not represent a bond with L_{101A} in the formula (101B) are independently
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are the same as defined in the formula (101)),
a halogen atom, a cyano group, a nitro group,
a substituted or unsubstituted aryl group having 6 to 50 ring carbon atoms, or
a substituted or unsubstituted monovalent heterocyclic group having 5 to 50 ring atoms.

27. The organic electroluminescence device according to claim 24, wherein any one of R_{111A} and R_{113A} to R_{116A} in the formula (101B) represents a bond with L_{101A} in the formula (101A).

28. The organic electroluminescence device according to claim 24, wherein L_{101A} in the formula (101A) is an unsubstituted aromatic hydrocarbon group having 6 to 30 ring carbon atoms, or an unsubstituted heterocyclic group having 5 to 30 ring atoms.

29. The organic electroluminescence device according to claim 24, wherein any one of R_{111A} and R_{113A} to R_{116A} in the formula (101B) represents a bond with L_{101A} in the formula (101A), and
R_{112A} is
a hydrogen atom,
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 9 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are the same as defined in the formula (101)),
a halogen atom, a cyano group, a nitro group,
an unsubstituted aryl group having 6 to 50 ring carbon atoms, or
a substituted or unsubstituted monovalent heterocyclic group having 5 to 50 ring atoms.

30. The organic electroluminescence device according to claim 24, wherein at least one of R₁₀₉ and R₁₁₀ in the formula (101) is the substituent R, or the group represented by the formula (101A).

31. The organic electroluminescence device according to claim 24, wherein at least one of R₁₀₁, R₁₀₄, R₁₀₅, and R₁₀₈ to R₁₁₀ in the formula (101) is the group represented by the formula (101A).

32. The organic electroluminescence device according to claim 24, wherein at least one of R₁₀₉ and R₁₁₀ in the formula (101) is the group represented by the formula (101A).

33. The organic electroluminescence device according to claim 24, wherein the compound represented by the formula (101) is the compound represented by the following formula (111): 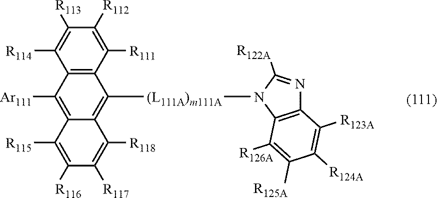
wherein in the formula (111),
one or more sets of the adjacent two or more of R₁₁₁ to R₁₁₈ form a substituted or unsubstituted, saturated or unsaturated ring by bonding with each other, or do not bond with each other;
R₁₁₁ to R₁₁₈ which do not bond with each other are independently a hydrogen atom or a substituent R;
the substituent R is the same as defined in the formula (101);
L_{111A} is
a substituted or unsubstituted aromatic hydrocarbon group having 6 to 30 ring carbon atoms, or
a substituted or unsubstituted heterocyclic group having 5 to 30 ring atoms;
m111A is an integer of 0 to 3; when m111A is 0, any of carbon atoms and nitrogen atoms in the benzimidazole and any of carbon atoms in the anthracene are directly bonded via a single bond;
when two or more L_{111A}’s are present, the two or more L_{111A}’s may be the same as or different from each other;
one or more sets of the adjacent two or more of m111A L_{111A}’s do not form a substituted or unsubstituted, saturated or unsaturated ring;
R_{122A} to R_{126A} are independently a hydrogen atom or a substituent R;
the substituent R is the same as defined in the formula (101);
Ar₁₁₁ is
a substituted or unsubstituted aryl group having 6 to 30 ring carbon atoms, or
a substituted or unsubstituted monovalent heterocyclic group having 5 to 30 ring atoms;
provided that the compound represented by the formula (111) comprises at least one deuterium atom.

34. An electronic apparatus, comprising the organic electroluminescence device according to claim 21.
